SEQUENCE LISTING (1074 sequences)

SEQ ID NO: 1 - protein, 6 aa, Artificial Sequence
  Synthetic Peptide
  MISC_FEATURE (2)..(2): Xaa = N, H, or K
  MISC_FEATURE (3)..(3): Xaa = R, S, D, E, Q, N, G, or Y
  MISC_FEATURE (4)..(4): Xaa = I, S, T, V, or L
  MISC_FEATURE (5)..(5): Xaa = L, F, N, Y, V, I, S, D, E, or A
RXXXXH

SEQ ID NO: 2 - protein, 6 aa, Artificial Sequence
  Synthetic Peptide
  MISC_FEATURE (2)..(2): Xaa = N or H
  MISC_FEATURE (3)..(3): Xaa = R, S, D, E, Q, N, G, Y, or H
  MISC_FEATURE (4)..(4): Xaa = I, S, T, V, or L
  MISC_FEATURE (5)..(5): Xaa = L, F, N, Y, V, I, S, D, E, or A
RXXXXH

SEQ ID NO: 3 - protein, 6 aa, Artificial Sequence
  Synthetic Peptide
  MISC_FEATURE (2)..(2): Xaa = N or K
  MISC_FEATURE (3)..(3): Xaa = R, S, D, E, Q, N, G, Y, or H
  MISC_FEATURE (4)..(4): Xaa = I, S, T, V, or L
  MISC_FEATURE (5)..(5): Xaa = L, F, N, Y, V, I, S, D, E, or A
RXXXXH

SEQ ID NO: 4 - RNA, 24 nt, Artificial Sequence
  Synthetic Oligonucleotide
gggaacaaagcugaaguacuuacc

SEQ ID NO: 5 - DNA, 18 bp, Artificial Sequence
  Synthetic Oligonucleotide
gggtagggcgggttggga

SEQ ID NO: 6 - DNA, 25 bp, Artificial Sequence
  Synthetic Oligonucleotide
  misc_feature (25)..(25): 3 prime thiol modification
ttataactattcctaaaaaaaaaaa

SEQ ID NO: 7 - DNA, 26 bp, Artificial Sequence
  Synthetic Oligonucleotide
  misc_feature (1)..(1): 5 prime thiol modification
aaaaaaaaaactcccctaataacaat

SEQ ID NO: 8 - RNA, 45 nt, Artificial Sequence
  Synthetic Oligonucleotide
ggguaggaauaguuauaauuucccuuucccauuguuauuagggag

SEQ ID NO: 9 - RNA, 12 nt, Artificial Sequence
  Synthetic Oligonucleotide
  misc_feature (1)..(1): 5 prime biotin tag
  misc_feature (12)..(12): 3 primer Iowas Black quencher
ucucguacguuc

SEQ ID NO: 10 - RNA, 24 nt, Artificial Sequence
  Synthetic Oligonucleotide
  misc_feature (1)..(1): 5 prime biotin tag
  misc_feature (24)..(24): 3 prime Iowa Black quencher
ucucguacguucucucguacguuc

SEQ ID NO: 11 - DNA, 12 bp, Artificial Sequence
  Synthetic Oligonucleotide
  misc_feature (1)..(12): n is a, c, g, or t
nnnnnnnnnnnn

SEQ ID NO: 12 - DNA, 66 bp, Artificial Sequence
  Synthetic Oligonucleotide
tgtggttggtgtggttggttcatggtcatattggtttttttttttttttccaaccacagtctctgt

SEQ ID NO: 13 - DNA, 35 bp, Artificial Sequence
  Synthetic Oligonucleotide
ggttggtagtctcgaattgctctctttcactggcc

SEQ ID NO: 14 - DNA, 48 bp, Artificial Sequence
  Synthetic Oligonucleotide
gaaattaatacgactcactatagggggttggttcatggtcatattggt

SEQ ID NO: 15 - DNA, 57 bp, Artificial Sequence
  Synthetic Oligonucleotide
gaaattaatacgactcactatagggggttggtgtggttggttcatggtcatattggt

SEQ ID NO: 16 - DNA, 31 bp, Artificial Sequence
  Synthetic Oligonucleotide
ggccagtgaaagagagcaattcgagactacc

SEQ ID NO: 17 - RNA, 64 nt, Artificial Sequence
  Synthetic Oligonucleotide
gauuuagacuaccccaaaaacgaaggggacuaaaacccagugaaagagagcaauucgagacuac

SEQ ID NO: 18 - RNA, 64 nt, Artificial Sequence
  Synthetic Oligonucleotide
gauuuagacuaccccaaaaacgaaggggacuaaaacaaagagagcaauucgagacuaccaacca

SEQ ID NO: 19 - RNA, 64 nt, Artificial Sequence
  Synthetic Oligonucleotide
gauuuagacuaccccaaaaacgaaggggacuaaaacagacuaccaaccacagagacugugguug

SEQ ID NO: 20 - DNA, 106 bp, Artificial Sequence
  Synthetic Oligonucleotide
gttagatcgcaagcatatcattgcgcttgcgatctaactgctgcgccgccgggaaaatactgtacggttagatcgcatagtctcgaattgctctctttcactggcc

SEQ ID NO: 21 - DNA, 71 bp, Artificial Sequence
  Synthetic Oligonucleotide
gttagatcgcaagcatatcattgcgcttgcgatctaactgctgcgccgccgggaaaatactgtacggttag

SEQ ID NO: 22 - DNA, 35 bp, Artificial Sequence
  Synthetic Oligonucleotide
atcgcatagtctcgaattgctctctttcactggcc

SEQ ID NO: 23 - DNA, 50 bp, Artificial Sequence
  Synthetic Oligonucleotide
gaaattaatacgactcactatagggatcgcaagcatatcattgcgcttgc

SEQ ID NO: 24 - DNA, 31 bp, Artificial Sequence
  Synthetic Oligonucleotide
ggccagtgaaagagagcaattcgagactatg

SEQ ID NO: 25 - RNA, 64 nt, Artificial Sequence
  Synthetic Oligonucleotide
gauuuagacuaccccaaaaacgaaggggacuaaaacccagugaaagagagcaauucgagacuau

SEQ ID NO: 26 - RNA, 64 nt, Artificial Sequence
  Synthetic Oligonucleotide
gauuuagacuaccccaaaaacgaaggggacuaaaacagagcaauucgagacuaugcgaucuaac

SEQ ID NO: 27 - RNA, 64 nt, Artificial Sequence
  Synthetic Oligonucleotide
gauuuagacuaccccaaaaacgaaggggacuaaaacacuaugcgaucuaaccguacaguauuuu

SEQ ID NO: 28 - RNA, 28 nt, Hepatitis delta virus
aggcccucgagaacaagaagaagcagcu

SEQ ID NO: 29 - DNA, 136 bp, Hepatitis delta virus
gccggctactcttctttcccttctctcgtcttcctcggtcaacctcctgagttcctcttcttcctccttgctgaggctcttccctcccgcggagagctgcttcttcttgttctcgagggccttccttcgtcggtga

SEQ ID NO: 30 - RNA, 28 nt, Adenovirus
cugcgccuccugcggugcggaugcauac

SEQ ID NO: 31 - DNA, 99 bp, Adenovirus
aatggattcgggggagtatgcatccgcaccgcaggaggcgcagacggtttcgcactccacgagccaggtcagatccggctcatcggggtcaaaaacaag

SEQ ID NO: 32 - RNA, 28 nt, Adenovirus
gaucggcucgcauccucgcaccgagcgu

SEQ ID NO: 33 - DNA, 146 bp, Adenovirus
gtaggtgacaaagagacgctcggtgcgaggatgcgagccgatcgggaagaactggatctcccgccaccagttggaggagtggctgttgatgtggtgaaagtagaagtccctgcgacgggccgaacactcgtgctggcttttgtaaa

SEQ ID NO: 34 - RNA, 28 nt, Adenovirus
cgcucucguacgagggaggaggagagga

SEQ ID NO: 35 - DNA, 130 bp, Adenovirus
gtgcgttctcttccttgttagagatgaggcgcgcggtggtgtcttcctctcctcctccctcgtacgagagcgtgatggcgcaggcgaccctggaggttccgtttgtgcctccgcggtatatggctcctac

SEQ ID NO: 36 - RNA, 28 nt, Adenovirus
aggagcgcacgcccuucucgcggucgcc

SEQ ID NO: 37 - DNA, 99 bp, Adenovirus
cctggcctacaactatggcgaccgcgagaagggcgtgcgctcctggacgctgctcaccacctcggacgtcacctgcggcgtggagcaagtctactggtc

SEQ ID NO: 38 - RNA, 28 nt, Adenovirus
cacacaaaaaagaacacagaucuucaug

SEQ ID NO: 39 - DNA, 127 bp, Adenovirus
ccagcgcttggattacatgaagatctgtgttcttttttgtgtgctaagtttaacaagtagcctaaggacttcacctacaaccgttggttccttacgtcagctacaagattccaccaaaggtacacac

SEQ ID NO: 40 - RNA, 28 nt, Torque teno virus
ggagauucucuuucuucuccgugagggg

SEQ ID NO: 41 - DNA, 61 bp, Torque teno virus
gctacagtaagatattacccctcacggagaagaaagagaatctccgttcgaggttgggagc

SEQ ID NO: 42 - RNA, 28 nt, Torque teno virus
uuugcuguacggaucggccgcccgauaa

SEQ ID NO: 43 - DNA, 138 bp, Torque teno virus
tgagtttttgctgctggaggacacagcacacggagctcagtaattgtgagtagcgaagtgtctgtgaggccgggcgggtgcagtaggcctaaagccgaatcaaggggcttatcgggcggccgatccgtacagcaaaac

SEQ ID NO: 44 - RNA, 28 nt, Torque teno virus
gacuucggugguuucacucaccuucggc

SEQ ID NO: 45 - DNA, 75 bp, Torque teno virus
tgatcttgggcgggagccgaaggtgagtgaaaccaccgaagtctaggggcaattcgggctagatcagtctggcgg

SEQ ID NO: 46 - RNA, 28 nt, Avian gyrovirus
ccuccucuuaacgcggcgaucaaaggau

SEQ ID NO: 47 - DNA, 141 bp, Avian gyrovirus
atatgcgcgtagaagatcctttgatcgccgcgttaagaggaggatcttcaacccacacccgggctcctatgtggtaaggctaccgaacccttacaataagcttaccctctttttccaaggcattgtattcattccggaggc

SEQ ID NO: 48 - RNA, 28 nt, Chicken anemia virus
accguugaugguccggguggaguaucuu

SEQ ID NO: 49 - DNA, 117 bp, Chicken anemia virus
tgaacgctctccaagaagatactccacccggaccatcaacggtgttcaggccaccaacaagttcacggccgttggaaacccctcactgcagagagatccggattggtatcgctggaa

SEQ ID NO: 50 - RNA, 28 nt, Torque teno virus
uuaauucugauugguuacacccuaugca

SEQ ID NO: 51 - DNA, 96 bp, Torque teno virus
gctcaagtcctcatttgcatagggtgtaaccaatcagaattaaggcgttcccagtaaagtgaatataagtaagtgcagttccgaatggctgagttt

SEQ ID NO: 52 - RNA, 28 nt, Torque teno virus
gccagaagcccucuaugaggcagguucu

SEQ ID NO: 53 - DNA, 91 bp, Torque teno virus
aagctccggtcatacaatggttccctcctagccggagaacctgcctcatagagggcttctggccgttgagctacggacactggttccgtac

SEQ ID NO: 54 - RNA, 28 nt, Arenavirus
uuaagucuagguuagguuugaaaaaauc

SEQ ID NO: 55 - DNA, 129 bp, Arenavirus
gacgtttggtggagtgattttttcaaacctaacctagacttaagataagatctcatcattgcattcacaacattgaaaggtacctcaattaacttgtgaatgtgccacgacagcaaagtggacacgtaa

SEQ ID NO: 56 - RNA, 28 nt, Mammarenavirus
gauaugaaaauggcuguuaacaauggug

SEQ ID NO: 57 - DNA, 116 bp, Mammarenavirus
atgaacaggacaagtcaccattgttaacagccattttcatatcacagattgcacgttcgaattccttttctgaattcaagcatgtgtatctcattgaactacccacagcttctgag

SEQ ID NO: 58 - RNA, 28 nt, Mammarenavirus
ugaggaaggugaugaguuggaauaggcc

SEQ ID NO: 59 - DNA, 99 bp, Mammarenavirus
aatctgatgagatgtggcctattccaactcatcaccttcctcattttggctggcagaagttgtgatggcatgatgattgatagaaggcacaatctcacc

SEQ ID NO: 60 - RNA, 28 nt, Mammarenavirus
acuauugauacaauuugugaucaaugug

SEQ ID NO: 61 - DNA, 132 bp, Mammarenavirus
  misc_feature (95)..(95): n is a, c, g, or t
cgacaccattagccacacattgatcacaaattgtatcaatagtttcagcaagttgtgttggagttttacacttgacattatgcaatgctgcaganacaaacttggttaacagaggtgtttcctcacccatga

SEQ ID NO: 62 - RNA, 28 nt, Mammarenavirus
ucguccuguaaauggacgcccccgugac

SEQ ID NO: 63 - DNA, 141 bp, Mammarenavirus
cgccgaaaggcggtgggtcacgggggcgtccatttacaggacgaccttggggcttgaggttctaaacaccatgtctctggggagaactgctctcaaaactggtatattgagtcctcctgacacagctgcatcatacattat

SEQ ID NO: 64 - RNA, 28 nt, Mammarenavirus
uguugacuuggcauaugcauaaacuugu

SEQ ID NO: 65 - DNA, 81 bp, Mammarenavirus
  misc_feature (80)..(80): n is a, c, g, or t
tcattgcattcacaacaggaaagggaacttcaacaagtttgtgcatgtgccaagttaacaaggtgctaacatgatccttnc

SEQ ID NO: 66 - RNA, 28 nt, Mammarenavirus
acaccauugcucacaaaguuuguugcug

SEQ ID NO: 67 - DNA, 89 bp, Mammarenavirus
ctgacaattgtgtgggtgttttacactttacattatgtaaagctgcagcaacaaactttgtgagcaatggtgtttcttcacccatgaca

SEQ ID NO: 68 - RNA, 28 nt, Mammarenavirus
ugucaaguugagugcagaagagucacgg

SEQ ID NO: 69 - DNA, 148 bp, Mammarenavirus
  misc_feature (11)..(11): n is a, c, g, or t
  misc_feature (26)..(26): n is a, c, g, or t
  misc_feature (58)..(58): n is a, c, g, or t
  misc_feature (79)..(79): n is a, c, g, or t
  misc_feature (89)..(89): n is a, c, g, or t
  misc_feature (116)..(116): n is a, c, g, or t
gatgctcaaanctcttccaaacaagntcttcaaaaattcgtgattcttctgcactcancttgacatcaacaattttcanatcttgtctnccatgcatatcaaaaagctttctaatntcatctgcaccttgtgcagtgaaaaccattga

SEQ ID NO: 70 - RNA, 28 nt, Mamastrovirus
caguccgugauaggcaguguucuacaua

SEQ ID NO: 71 - DNA, 119 bp, Mamastrovirus
ctccatgggaagctcctatgctatcagttgcttgctgcgttcatggcagaagatcacccttttaaggtgtatgtagaacactgcctatcacggactgcaaagcagcttcgtgactctgg

SEQ ID NO: 72 - RNA, 28 nt, Norwalk virus
gaucgcccucccacgugcucagaucuga

SEQ ID NO: 73 - DNA, 96 bp, Norwalk virus
agccaatgttcagatggatgagattctcagatctgagcacgtgggagggcgatcgcaatctggctcccagttttgtgaatgaagatggcgtcgaat

SEQ ID NO: 74 - RNA, 28 nt, Sapporo virus
agucaucaccauagguguggacagucuc

SEQ ID NO: 75 - DNA, 164 bp, Sapporo virus
gggctcccatctggcatgccattcaccagtgtcatcaattcwgtcaaccacatgatatactttgccgcggctgtgctgcaggcctatgaggaacacaatgtgccatacactggcaatgtgttccagattgagactgtccacacctatggtgatgactgcatgta

SEQ ID NO: 76 - RNA, 28 nt, Human coronovirus
augggcacaauaaccaacuugcacacca

SEQ ID NO: 77 - DNA, 89 bp, Human coronavirus
tagtgtcaaacgtgatggtgtgcaagttggttattgtgcccatggtattaagtactattcacgtgttagaagtgttagcggtagagcta

SEQ ID NO: 78 - RNA, 28 nt, Human coronavirus
aauggugaaccaaacgcccuauacacag

SEQ ID NO: 79 - DNA, 144 bp, Human coronavirus
gtggtgaatggaatgctgtgtatagggcgtttggttcaccatttattacaaatggtatgtcattgctagatataattgttaaaccagttttctttaatgcttttgttaaatgcaattgtggttctgagagttggagtgttggtg

SEQ ID NO: 80 - RNA, 28 nt, Human coronavirus
gcuugaccaguagaggggcauaacccac

SEQ ID NO: 81 - DNA, 121 bp, Human coronavirus
tgaagtcagatgagggtgggttatgcccctctactggtcaagcgatggaaagtgttggattcgtttatgataatcatgtgaagatagattgtcgctgcattcttggacaagaatggcatgt

SEQ ID NO: 82 - RNA, 28 nt, Betacoronavirus
gcuuccugauaggcuuucugcgcagcuu

SEQ ID NO: 83 - DNA, 76 bp, Betacoronavirus
cctttgctgagttggaagctgcgcagaaagcctatcaggaagctatggactctggtgacacctcaccacaagttct

SEQ ID NO: 84 - RNA, 28 nt, Betacoronavirus
uguccucaccugcauuuagguuaggucc

SEQ ID NO: 85 - DNA, 115 bp, Betacoronavirus
tgtctgcatgttgttggacctaacctaaatgcaggtgaggacatccagcttcttaaggcagcatatgaaaatttcaattcacaggacatcttacttgcaccattgttgtcagcag

SEQ ID NO: 86 - RNA, 28 nt, Reston ebolavirus
gacaauuaggaguccugaaaagcgagcc

SEQ ID NO: 87 - DNA, 92 bp, Reston ebolavirus
taattcagttgctcaggctcgcttttcaggactcctaattgtcaaaaccgttcttgatcatattctgcaaaaaaccgaccaaggagtaagac

SEQ ID NO: 88 - RNA, 28 nt, Sudan ebolavirus
cuuugcaacacuuuaggaaugcccccaa

SEQ ID NO: 89 - DNA, 81 bp, Sudan ebolavirus
tagtcaatcccccatttgggggcattcctaaagtgttgcaaaggtatgtgggtcgtattgctttgccttttcctaacctgg

SEQ ID NO: 90 - RNA, 28 nt, Zaire ebolavirus
ugacuguuuuucuguuguccacccuugg

SEQ ID NO: 91 - DNA, 72 bp, Zaire ebolavirus
tgcctaacagatcgaccaagggtggacaacagaaaaacagtcaaaagggccagcatacagagggcagacaga

SEQ ID NO: 92 - RNA, 28 nt, Marburgvirus
ggcuugucuucucugggacuuuuucgac

SEQ ID NO: 93 - DNA, 81 bp, Marburgvirus
cttcatcaactgagggtcgaaaaagtcccagagaagacaagcctgtttaggatttcgcttcctgccgacatgttctcagta

SEQ ID NO: 94 - RNA, 28 nt, Bagaza virus
ugucauugauauggguaugcgacauggu

SEQ ID NO: 95 - DNA, 123 bp, Bagaza virus
ttctggatctgatggaccatgtcgcatacccatatcaatgacagccaaccttcaggatttgaccccgataggaaggctcataacggtcaatccatatgtgtctacatcatcatcggggacaaa

SEQ ID NO: 96 - RNA, 28 nt, Culex flavivirus
gggaacagcacguggucgaggagguaug

SEQ ID NO: 97 - DNA, 114 bp, Culex flavivirus
agctgtgggaatcgacatacctcctcgaccacgtgctgttcccgatgtacgtgatgttggcgttcaatctgaaatcacagttcgtacctgtggactcgatggtactgctgaact

SEQ ID NO: 98 - RNA, 28 nt, Dengue virus
uugacacgcgguuucucgcgcguuucag

SEQ ID NO: 99 - DNA, 72 bp, Dengue virus
ccgtctttcaatatgctgaaacgcgcgagaaaccgcgtgtcaactgtttcacagttggcgaagagattctca

SEQ ID NO: 100 - RNA, 28 nt, Japanese encephalitis virus
uguuccauuccauuuucggucaaaccuc

SEQ ID NO: 101 - DNA, 133 bp, Japanese encephalitis virus
gtgtgaaagaagaccgcatagcttacggaggcccatggaggtttgaccgaaaatggaatggaacagatgacgtgcaagtgatcgtggtagaaccggggaaggctgcagtaaacatccagacaaaaccaggagt

SEQ ID NO: 102 - RNA, 28 nt, Kyasanur Forest disease virus
cuuuaagccacuuaugcccucuuccggu

SEQ ID NO: 103 - DNA, 143 bp, Kyasanur Forest disease virus
ttccagtgcatgctcatagtgatcttaccggaagagggcataagtggcttaaaggggactcagtcaagacgcatctgacacgtgtggaaggctgggtatggaagaataagctcctgacgatggccttttgtgcagttgtgtgg

SEQ ID NO: 104 - RNA, 28 nt, Murray Valley encephalitis virus
cacuaaugggaauacgcgggguaugccg

SEQ ID NO: 105 - DNA, 138 bp, Murray Valley encephalitis virus
caatatgctaaaacgcggcataccccgcgtattcccattagtgggagtgaagagggtagtaatgaacttgctagatggcagagggccaatacggtttgtgttggctctcttagctttcttcaggtttacagcacttgc

SEQ ID NO: 106 - RNA, 28 nt, Powassan virus
cuccaucaacccccaucaucaugcgccu

SEQ ID NO: 107 - DNA, 109 bp, Powassan virus
gttggggcaagtcaatcttgtggagtgtgcctgaaagtcctaggcgcatgatgatgggggttgatggagctggggagtgccccctgcacaagagagcaacaggagtgtt

SEQ ID NO: 108 - RNA, 28 nt, Saint Louis encephalitis virus
ccacggccauccagcagacuuccaagua

SEQ ID NO: 109 - DNA, 137 bp, Saint Louis encephalitis virus
cggggttgaagaggatacttggaagtctgctggatggccgtggacccgtgcggttcatactagccattctgacattcttccgatttacagctctacagccaactgaggcgctgaagcgcagatggagggctgtagat

SEQ ID NO: 110 - RNA, 28 nt, Tembusu virus
cuuccagaacgacaucgauccacucaac

SEQ ID NO: 111 - DNA, 122 bp, Tembusu virus
gagggagtgaatggtgttgagtggatcgatgtcgttctggaaggaggctcatgtgtgaccatcacggcaaaagacaggccgaccatagacgtcaagatgatgaacatggaggctacggaatt

SEQ ID NO: 112 - RNA, 28 nt, Tick-borne encephalitis virus
gagggggaccgccccccuuuccuuucag

SEQ ID NO: 113 - DNA, 84 bp, Tick-borne encephalitis virus
gagaacaagagctggggatggccaggaaggccattctgaaaggaaaggggggcggtccccctcgacgagtgtcgaaagagaccg

SEQ ID NO: 114 - RNA, 28 nt, Usutu virus
uuaggauugugggccuccccaguuguug

SEQ ID NO: 115 - DNA, 144 bp, Usutu virus
ctgtctccaactgtccaacaactggggaggcccacaatcctaagagagctgaggacacgtacgtgtgcaaaagtggtgtcactgacaggggctggggcaatggctgtggactatttggcaaaggaagtatagacacgtgtgcca

SEQ ID NO: 116 - RNA, 28 nt, West Nile virus
gagggugguuguaaaggcuuugccaaug

SEQ ID NO: 117 - DNA, 85 bp, West Nile virus
caagtctggaagcagcattggcaaagcctttacaaccaccctcaaaggagcgcagagactagccgctctaggagacacagcttgg

SEQ ID NO: 118 - RNA, 28 nt, Yellow fever virus
uccaaauguguuuauugccuagcaacuc

SEQ ID NO: 119 - DNA, 139 bp, Yellow fever virus
attggtctgcaaatcgagttgctaggcaataaacacatttggattaattttaatcgttcgttgagcgattagcagagaactgaccagaacatgtctggtcgtaaagctcagggaaaaaccctgggcgtcaatatggtac

SEQ ID NO: 120 - RNA, 28 nt, Zika virus
gaccaaguauaugacuuuuuggcucguu

SEQ ID NO: 121 - DNA, 147 bp, Zika virus
aaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaa

SEQ ID NO: 122 - RNA, 28 nt, Hepacivirus C
ugacguccugugggcggcgguugguguu

SEQ ID NO: 123 - DNA, 121 bp, Hepacivirus C
tgagcacaaatcctaaacctcaaagaaaaaccaaaagaaacaccaaccgtcgcccacaggacgtcaagttcccgggtggcggtcagatcgttggtggagtttacttgttgccgcgcagggg

SEQ ID NO: 124 - RNA, 28 nt, Pegivirus A
ucagcugcgacggcugcgguguaggggc

SEQ ID NO: 125 - DNA, 98 bp, Pegivirus A
ggtacgggttggagcctgacctggctgcgtctttgctaagactatacgacgactgcccctacaccgcagccgtcgcagctgacattggtgaagcctct

SEQ ID NO: 126 - RNA, 28 nt, Pegivirus C
guguuucccggcacaucguccgcugaac

SEQ ID NO: 127 - DNA, 112 bp, Pegivirus C
  misc_feature (89)..(89): n is a, c, g, or t
atgtcagctgggcaaaagtacgcggcgtcaactggcccctcctggtgggtgttcagcggacgatgtgccgggaaacactgtctcccggnccatcggatgacccccaatgggc

SEQ ID NO: 128 - RNA, 28 nt, Pegivirus H
caccacagcgaauaacaggccucgagau

SEQ ID NO: 129 - DNA, 121 bp, Pegivirus H
ggtggccatcaagctatctcgaggcctgttattcgctgtggtgttggcgcacggagtgtgccgacctgggcgggtatttggtcttgaggtttgcgcggacatctcttggttggtggagttt

SEQ ID NO: 130 - RNA, 28 nt, Orthohantavirus
caucaggcucaagcccuguuggaucaac

SEQ ID NO: 131 - DNA, 92 bp, Orthohantavirus
ctggctacaaaaccagttgatccaacagggcttgagcctgatgaccatctgaaggagaaatcatctctgagatatgggaatgtcctggatgt

SEQ ID NO: 132 - RNA, 28 nt, Orthohantavirus
uagucuauacacucuacugcugucagug

SEQ ID NO: 133 - DNA, 109 bp, Orthohantavirus
cctttccagttgggtcactgacagcagtagagtgtatagactacctggatcgtctctatgcaataaggcatgacattgttgaccagatgataaagcatgactggtcaga

SEQ ID NO: 134 - RNA, 28 nt, Orthohantavirus
uauacuggacaacaccaucauuucuucu

SEQ ID NO: 135 - DNA, 124 bp, Orthohantavirus
acacaatggcccagtagaagaaatgatggtgttgtccagtatatgaggctagttcaagctgagataagttatgttagagagcacttgatcaaaactgaggagagagctgcactagaagccatgt

SEQ ID NO: 136 - RNA, 28 nt, Orthohantavirus
ugaaucuagcaaauugauacauucuacu

SEQ ID NO: 137 - DNA, 72 bp, Orthohantavirus
aggcacaataggagcagtagaatgtatcaatttgctagattcgctgtatatggtccgccatgacctaattga

SEQ ID NO: 138 - RNA, 28 nt, Orthohantavirus
ucugccauguugugguagugcugaugcu

SEQ ID NO: 139 - DNA, 133 bp, Orthohantavirus
tagagcactaatcacagcatcagcactaccacaacatggcagatatagagaggctaatagcggagggccttgaaatagaaaaggagcttatgacagctcgtattcgtttacaggaggcaaaggaggctgcaga

SEQ ID NO: 140 - RNA, 28 nt, Orthohantavirus
cuggcaacaacaaguuguuguucauggc

SEQ ID NO: 141 - DNA, 136 bp, Orthohantavirus
aagaggatataacccgccatgaacaacaacttgttgttgccagacaaaaacttaaggatgcagagagagcagtggaaatggacccagatgacgttaacaaaaacacactgcaagcaaggcaacaaacagtgtcagc

SEQ ID NO: 142 - RNA, 28 nt, Orthohantavirus
uacuuauuuaagauacuauuagcaacca

SEQ ID NO: 143 - DNA, 111 bp, Orthohantavirus
tcacaaagtctcaggtggttgctaatagtatcttaaataagtattgggaagagccatattttagccaaacaaggaatattagtttaaaaggtatgtcaggccaagtacaag

SEQ ID NO: 144 - RNA, 28 nt, Orthohantavirus
cccgaguuugguuuccaaugcagacaca

SEQ ID NO: 145 - DNA, 133 bp, Orthohantavirus
cacattacagagcagacgggcagctgtgtctgcattggagaccaaactcggagaactcaaacgggagctggctgatcttattgcagctcagaaattggcttcaaaacctgttgatccaacagggattgaacct

SEQ ID NO: 146 - RNA, 28 nt, Orthohantavirus
uaguuuuugagaggauucuguuaaugcc

SEQ ID NO: 147 - DNA, 98 bp, Orthohantavirus
  misc_feature (43)..(43): n is a, c, g, or t
caaccaaactgagaaggcattaacagaatcctctcaaaaactnattcaggagatcgaccaggctggacaaaatccggattccattcagcagcagtcta

SEQ ID NO: 148 - RNA, 28 nt, Orthohantavirus
auuuguccuccaaugcugacacagcugc

SEQ ID NO: 149 - DNA, 136 bp, Orthohantavirus
ccgacccggatgatgttaacaagagtacactacagagcagacgggcagctgtgtcagcattggaggacaaactggcagacttcaagagacagcttgcagatctggtatcaagtcaaaaaatgggtgaaaagcctgt

SEQ ID NO: 150 - RNA, 28 nt, Hepatitis B virus
acggacugaggcccacucccauaggaau

SEQ ID NO: 151 - DNA, 84 bp, Hepatitis B virus
gcacctgtattcccatcccatcatcctgggctttcgcaaaattcctatgggagtgggcctcagtccgtttctcctggctcagtt

SEQ ID NO: 152 - RNA, 28 nt, Orthohepevirus
ccacgacggcggccagacggcuggccgg

SEQ ID NO: 153 - DNA, 115 bp, Orthohepevirus
tgcctatgctgcccgcgccaccggccggtcagccgtctggccgccgtcgtgggcggcgcagcggcggtgccggcggtggtttctggggtgacagggttgattctcagcccttcgc

SEQ ID NO: 154 - RNA, 28 nt, Cytomegalovirus
auauucucgugagaacuuugagauucgc

SEQ ID NO: 155 - DNA, 75 bp, Cytomegalovirus
taagaggtttcaagtgcgaatctcaaagttctcacgagaatattgtcttcaagaatcgacaactgtggtccaaga

SEQ ID NO: 156 - RNA, 28 nt, Lymphocryptovirus
gaagacggcagaaagcagagucugggaa

SEQ ID NO: 157 - DNA, 125 bp, Lymphocryptovirus
gtgtctgtggttgtcttcccagactctgctttctgccgtcttcggtcaagtaccagctggtggtccgcatgttttgatccaaactttagttttaggatttatgcatccattatcccgcagttcca

SEQ ID NO: 158 - RNA, 28 nt, Rhadinovirus
cacgauuggccaagacaacaaaaaaccc

SEQ ID NO: 159 - DNA, 149 bp, Rhadinovirus
agccattatacacacgggttttttgttgtcttggccaatcgtgtctccatggcgctaaagggaccacaaaccctcgaggaaaatattgggtctgcggcccccactggtccctgcgggtacctctatgcctatctgacacacaacttccc

SEQ ID NO: 160 - RNA, 28 nt, Herpes simplex virus
gcgccgcuagcaucuucguggccgcguu

SEQ ID NO: 161 - DNA, 137 bp, Herpes simplex virus
acgtacacaaactcgaacgcggccacgaagatgctagcggcgcagtggggcgcccccaggcatttggcacagagaaacgcgtaatcggccacccactggggcgagaggcggtaggtttgcttgtacagctcgatggt

SEQ ID NO: 162 - RNA, 28 nt, Herpes simplex virus
uggaaacguucgcgaccacgggagacgu

SEQ ID NO: 163 - DNA, 95 bp, Herpes simplex virus
gtgaaaaaggcagagacgtctcccgtggtcgcgaacgtttccaggtggcccaggagccgctccccctcgcgccacgcgtactccaggagcaactc

SEQ ID NO: 164 - RNA, 28 nt, Varicellovirus
aguagagcuuauaucuuauguuagacca

SEQ ID NO: 165 - DNA, 87 bp, Varicellovirus
atccttggttggttttggtctaacataagatataagctctactatagcgagcgtgcatacaacaacccaggccagaatccgaatgta

SEQ ID NO: 166 - RNA, 28 nt, Crimean Congo hemorrhagic fever virus
gagggaacauuuuucuuucugucaccgg

SEQ ID NO: 167 - DNA, 89 bp, Crimean Congo hemorrhagic fever virus
cctgaatctgtggaggcagtgccggtgacagaaagaaagatgttccctctgcctgagactccactgagtgaggtgcattcaatagagcg

SEQ ID NO: 168 - RNA, 28 nt, Orthonairovirus
gggcuccuugagcucucauggcacuuga

SEQ ID NO: 169 - DNA, 133 bp, Orthonairovirus
cccttgaactagccaagcagtcaagtgccatgagagctcaaggagcccagattgacactgtttttagcagctactactggctttggaaggcaggtgtgactgcagagatgttcccgacagtctcacagtttct

SEQ ID NO: 170 - RNA, 28 nt, Influenza virus
uuauggccauaugguccacugugguuuu

SEQ ID NO: 171 - DNA, 134 bp, Influenza virus
tctaatgtcgcagtctcgcactcgcgagatactgacaaaaaccacagtggaccatatggccataattaagaagtacacatcggggagacaggaaaagaacccgtcacttaggatgaaatggatgatggcaatga

SEQ ID NO: 172 - RNA, 28 nt, Influenza virus
gggaacaccgguguaugggaauguuguu

SEQ ID NO: 173 - DNA, 96 bp, Influenza virus
acaggcagcaatttcaacaacattcccatacaccggtgttcccccttattcccatggaacgggaacaggctacacaatagacaccgtgatcagaac

SEQ ID NO: 174 - RNA, 28 nt, Influenza virus
guagcauggggccaaaagauagaguuuu

SEQ ID NO: 175 - DNA, 124 bp, Influenza virus
atctgctttaggaggaccattagggaaaactctatcttttggccccatgctactcaagaaaatttctggttccggagtaaaagttaaagatacagtatatatccaaggtgtcagagcagtacaa

SEQ ID NO: 176 - RNA, 28 nt, Alphapapillomavirus
cucuggcguuccaacaaccaucugcgua

SEQ ID NO: 177 - DNA, 135 bp, Alphapapillomavirus
  misc_feature (62)..(62): n is a, c, g, or t
  misc_feature (64)..(64): n is a, c, g, or t
  misc_feature (92)..(92): n is a, c, g, or t
cagtgggtatggcaatacgcagatggttgttggaacgccagaggaggtaacgggggatgagnanagccaaggggggcggccggtggaggatnaggaggaggagcgtcaagggggagacggagaggcagatctaac

SEQ ID NO: 178 - RNA, 28 nt, Alphapapillomavirus
aaggguuuccuucggugucugcaucuuc

SEQ ID NO: 179 - DNA, 75 bp, Alphapapillomavirus
tccagattagatttgcacgaggaagaggaagatgcagacaccgaaggaaaccctttcggaacgtttaagtgcgtt

SEQ ID NO: 180 - RNA, 28 nt, Alphapapillomavirus
cgcauguguuuccaauagucuauauggu

SEQ ID NO: 181 - DNA, 85 bp, Alphapapillomavirus
gtacagacctacgtgaccatatagactattggaaacacatgcgcctagaatgtgctatttattacaaggccagagaaatgggatt

SEQ ID NO: 182 - RNA, 28 nt, Betapapillomavirus
ccaaagccuuuuaaaaaaagauuuccag

SEQ ID NO: 183 - DNA, 114 bp, Betapapillomavirus
tgaacttactgaccaaagctggaaatctttttttaaaaggctttggaaacaattagagctgagtgaccaagaagacgagggcgaggatggagaatctcagcgagcgtttcaatg

SEQ ID NO: 184 - RNA, 28 nt, Betapapillomavirus
cuuguagugcauugaaacguucgcugag

SEQ ID NO: 185 - DNA, 91 bp, Betapapillomavirus
taaaaggctttggacacaattagagctcagtgatcaagaagacgagggagaggatggaaacactcagcgaacgtttcaatgcactgcaaga

SEQ ID NO: 186 - RNA, 28 nt, Avulavirus
aggugcaggaguauugucuuggcucugc

SEQ ID NO: 187 - DNA, 143 bp, Avulavirus
gagtcacaaccatcagctggtgcaacccctcatgcgctccagtcagggcagagccaagacaatactcctgtacctgtggatcatgtccagctacctgtcgactttgtgcaggcgatgatgtctatgatggaggcattatcaca

SEQ ID NO: 188 - RNA, 28 nt, Avulavirus
ugaggcgagcaaggauugaguccggauc

SEQ ID NO: 189 - DNA, 63 bp, Avulavirus
ttcctcaacacttacgggtttatctatgacactacaccggacaagacaactttttccacccca

SEQ ID NO: 190 - RNA, 28 nt, Avulavirus
cgacuccggacccggaguccaccagcuu

SEQ ID NO: 191 - DNA, 97 bp, Avulavirus
aaaatcgtgagggggaagctggtggactccgggtccggagtcggtggacctgagtctagtagcttccctgctgtgccaagatgtcgtcagtgttcac

SEQ ID NO: 192 - RNA, 28 nt, Henipavirus
uacuuccuccugguugauagaaucauug

SEQ ID NO: 193 - DNA, 129 bp, Henipavirus
cactactcccgaggacaatgattctatcaaccaggaggaagtagttggggacccgtctgatcagggtttagagcatcctttccctttggggaaattcccggagaaagaagaaactcctgatgtacgcag

SEQ ID NO: 194 - RNA, 28 nt, Henipavirus
gcaaagcuccacaauaauggguaaccuc

SEQ ID NO: 195 - DNA, 112 bp, Henipavirus
ctaaatttgcccctggaggttacccattattgtggagctttgccatgggtgtggctactactattgacaggtctatgggggcattgaatatcaatcgtggttatcttgagcc

SEQ ID NO: 196 - RNA, 28 nt, Morbillivirus
ccaaaaccagguauagcuaucauaaugc

SEQ ID NO: 197 - DNA, 90 bp, Morbillivirus
aggggcatctatcaagcattatgatagctatacctggttttgggaaggacactggagaccctacggcaaatgtcgacattaacccagagc

SEQ ID NO: 198 - RNA, 28 nt, Morbillivirus
aucccucgagaugcaaaagucaauucuc

SEQ ID NO: 199 - DNA, 134 bp, Morbillivirus
aagctggtaatcctggagaattgacttttgcatctcgagggattaatttagataagcaagctcaacaatactttaaactggctgagaaaaatgatcaggggtattatgttagcttaggatttgagaacccacca

SEQ ID NO: 200 - RNA, 28 nt, Morbillivirus
uuuuucccgaucggcuuuaguugaaauu

SEQ ID NO: 201 - DNA, 129 bp, Morbillivirus
gacagctgctgaaggaatttcaactaaagccgatcgggaaaaagatgagctcagccgtcgggtttgttcctgacaccggccctgcatcacgcagtgtaatccgctccattataaaatccagccggctag

SEQ ID NO: 202 - RNA, 28 nt, Morbillivirus
uucaccgcugugaucagaaacaugauaa

SEQ ID NO: 203 - DNA, 143 bp, Morbillivirus
agagaaagcaacagctgtgatggggagctgggagcactcatggatgacctcccagtgcacaataccgaggtacagtgttatcatgtttctgatcacagcggtgaaaaggttgagggagtcgaagatgctgactctatcctggt

SEQ ID NO: 204 - RNA, 28 nt, Morbillivirus
cagaguauacuucguucuucuuucuucu

SEQ ID NO: 205 - DNA, 99 bp, Morbillivirus
cacgtgggcaactttagaagaaagaagaacgaagtatactctgctgattactgcaaaatgaagattgaaaagatgggtttagtttttgccctgggagga

SEQ ID NO: 206 - RNA, 28 nt, Respirovirus
cuguaauaauguaaucgcccuuucugua

SEQ ID NO: 207 - DNA, 78 bp, Respirovirus
gaggacacagaagagagcactcgatttacagaaagggcgattacattattacagaatcttggtgtaatccaatctgca

SEQ ID NO: 208 - RNA, 28 nt, Respirovirus
ucuacuguccaauuauccuguuaaauuc

SEQ ID NO: 209 - DNA, 143 bp, Respirovirus
ctgcagggataggaggaatttaacaggataattggacagtagaaaccagatcaaaagtaagaaaaacttagggtgaatgacaattcacagatcagctcaaccagacatcatcagcatacacgaaaccaaccttcacagtggat

SEQ ID NO: 210 - RNA, 28 nt, Respirovirus
ccuaaacaugauggauacccaaacgugu

SEQ ID NO: 211 - DNA, 102 bp, Respirovirus
ttgaagaccttgtccacacgtttgggtatccatcatgtttaggagctattataatacagatctggatagttttggtcaaagctatcactagcatctcagggt

SEQ ID NO: 212 - RNA, 28 nt, Respirovirus
ugagacugugcuccucuggccggggaua

SEQ ID NO: 213 - DNA, 111 bp, Respirovirus
  misc_feature (107)..(107): n is a, c, g, or t
gggaggaggtgctgttatccccggccagaggagcacagtctcagtgttcgtactaggcccaagtgtgactgatgatgcagacaagttattcattgcaaccaccttcntagc

SEQ ID NO: 214 - RNA, 28 nt, Rubulavirus
ccgcagaugcuggggcaggauccgcaug

SEQ ID NO: 215 - DNA, 98 bp, Rubulavirus
gcaagttcacctgcacatgcggatcctgccccagcatctgcggagaatgtgagggagatcattgagctcttaaaggggcttgatcttcgccttcagac

SEQ ID NO: 216 - RNA, 28 nt, Rubulavirus
uaguuucugaucaauggauccuggacac

SEQ ID NO: 217 - DNA, 114 bp, Rubulavirus
  misc_feature (74)..(74): n is a, c, g, or t
ccatgggagttggaagtgtccaggatccattgatcagaaactatcagtttggaaggaacttcttaaataccagntattttcagtatggtgttgagactgcaatgaaacaccagg

SEQ ID NO: 218 - RNA, 28 nt, Rubulavirus
aaauagagauugaggauugagccaauga

SEQ ID NO: 219 - DNA, 133 bp, Rubulavirus
aggcccaagatgctatcattggctcaatcctcaatctctatttgaccgagttgacaactatcttccacaatcaaattacaaaccctgcattgagtcctattacaattcaagctttaaggatcctactggggag

SEQ ID NO: 220 - RNA, 28 nt, Rubulavirus
uugcaggaguggaaucuugcugcggcag

SEQ ID NO: 221 - DNA, 87 bp, Rubulavirus
tatgctcacctatcactgccgcagcaagattccactcctgcaaatgtgggaattgcccagcaaagtgcgatcagtgcgaacgagatt

SEQ ID NO: 222 - RNA, 28 nt, Erythroparvovirus
cgccuggggugaugagguuaaaaaagcu

SEQ ID NO: 223 - DNA, 140 bp, Erythroparvovirus
gaactcagtgaaagcagcttttttaacctcatcaccccaggcgcctggaacactgaaaccccgcgctctagtacgcccatccccgggaccagttcaggagaatcatttgtcggaagcccagtttcctccgaagttgtagc

SEQ ID NO: 224 - RNA, 28 nt, Orthobunyavirus
auuugaccccugcaaaaguaagaucgac

SEQ ID NO: 225 - DNA, 101 bp, Orthobunyavirus
cataagacgccacaaccaagtgtcgatcttacttttgcaggggtcaaatttacagtggttaataaccattttccccagtacactgcaaatccagtgtcaga

SEQ ID NO: 226 - RNA, 28 nt, Orthobunyavirus
cguccuuuaauguagaagauucgaaugu

SEQ ID NO: 227 - DNA, 124 bp, Orthobunyavirus
ttaagcgtatccacaccactgggcttagttatgaccacattcgaatcttctacattaaaggacgcgagattaaaactagtctcgcaaaaagaagtgaatgggaggttacgcttaaccttggggg

SEQ ID NO: 228 - RNA, 28 nt, Orthobunyavirus
cuguuuccaggaaaaugauuauugacaa

SEQ ID NO: 229 - DNA, 89 bp, Orthobunyavirus
aaatttggagagtggcaggtggaggttgtcaataatcattttcctggaaacaggaacaacccaattggtaacaacgatcttaccatcca

SEQ ID NO: 230 - RNA, 28 nt, Orthobunyavirus
acuuacucuaugaagugugaaugaauca

SEQ ID NO: 231 - DNA, 101 bp, Orthobunyavirus
cagtccagtcctcgatgattcattcacacttcatagagtaagtggttacctggcaaggtacttacttgaaagatatttaactgtatcagcacctgagcaag

SEQ ID NO: 232 - RNA, 28 nt, Orthobunyavirus
ugccuccggaucaaauguagauguaguc

SEQ ID NO: 233 - DNA, 83 bp, Orthobunyavirus
cgatgtaccacaacggactacatctacatttgatccggaggcagcatatgtggcatttgaagctagatacggacaagtgctca

SEQ ID NO: 234 - RNA, 28 nt, Orthobunyavirus
cucucuaccaaaguagucaugucuagcc

SEQ ID NO: 235 - DNA, 139 bp, Orthobunyavirus
tgctgatcttctcatggctagacatgactactttggtagagaggtatgttattacctggatatcgaattccggcaggatgttccagcttacgacatacttcttgaatttctgccagctggcactgctttcaacattcgc

SEQ ID NO: 236 - RNA, 28 nt, Orthobunyavirus
auaaaugccacauacccgaccuccgggu

SEQ ID NO: 237 - DNA, 133 bp, Orthobunyavirus
atctcgctacgtttaacccggaggtcgggtatgtggcatttattgctaaacatggggcccaactcaatttcgataccgttagagtcttcttcctcaatcagaagaaggccaagatggtactcagtaagacggc

SEQ ID NO: 238 - RNA, 28 nt, Phlebovirus
gauaauucagcaccuauuaaugagacca

SEQ ID NO: 239 - DNA, 76 bp, Phlebovirus
ggctcttggtgtcaaatggtttcactaattggtgcagaattatcagcatcagttaaacagcatgtggggaaaggcc

SEQ ID NO: 240 - RNA, 28 nt, Phlebovirus
ucagaagcaaagaacuucccuauggacc

SEQ ID NO: 241 - DNA, 142 bp, Phlebovirus
tggagacaatagccaggtccatagggaagttctttgcttctgataccctctgtaacccccccaataaagtgaaaattcctgagacacatggcatcagggctcggaagcaatgtaaggggcctgtgtggacttgtgcaacatc

SEQ ID NO: 242 - RNA, 28 nt, Phlebovirus
ggcaucgacagucacaucuaggucuggc

SEQ ID NO: 243 - DNA, 132 bp, Phlebovirus
caaatctacgacaggccagggctgccagacctagatgtgactgtcgatgccacaggtgtgacagtggacataggggctgtgccagactcagcatcacaactgggttcatcaatcaatgctgggttgatcaca

SEQ ID NO: 244 - RNA, 28 nt, Phlebovirus
ucacauggguaccugcugcagaaauauu

SEQ ID NO: 245 - DNA, 128 bp, Phlebovirus
  misc_feature (114)..(114): n is a, c, g, or t
ttgagtcatgcaaaggtgttactacatcatcagcctctaagtgctctggggatgaatatttctgcagcaggtacccatgtgaaacagcaaatgttgaagcccactgcattctangaaggcatagtgca

SEQ ID NO: 246 - RNA, 28 nt, Phlebovirus
agagaggucacuugccaugccuuggaag

SEQ ID NO: 247 - DNA, 143 bp, Phlebovirus
  misc_feature (31)..(31): n is a, c, g, or t
  misc_feature (85)..(85): n is a, c, g, or t
  misc_feature (102)..(102): n is a, c, g, or t
  misc_feature (122)..(122): n is a, c, g, or t
  misc_feature (133)..(133): n is a, c, g, or t
atggggcccagcatgctacatcagttctgtnaagcctatggtgtacaccttccaaggcatggcaagtgacctctctaggtttganctgactagtttctctangagaggactgccaaatgttntgaaagctctnagctggccac

SEQ ID NO: 248 - RNA, 28 nt, Phlebovirus
  misc_feature (12)..(12): n is a, c, g, or u
ugggccagcucnaaaauccuccucagga

SEQ ID NO: 249 - DNA, 84 bp, Phlebovirus
  misc_feature (35)..(35): n is a, c, g, or t
  misc_feature (47)..(47): n is a, c, g, or t
  misc_feature (56)..(56): n is a, c, g, or t
  misc_feature (64)..(64): n is a, c, g, or t
gatttgatgctgctgtggtcctgaggaggattttngagctggcccanaaagctggnctggacanggaccagatgatgagggaca

SEQ ID NO: 250 - RNA, 28 nt, Picornavirus
uguuaccucgggguaccugaagggcauc

SEQ ID NO: 251 - DNA, 131 bp, Picornavirus
tggtgacaggctaaggatgcccttcaggtaccccgaggtaacacgcgacactcgggatctgagaaggggactggggcttctttaaaagcgcccagtttaaaaagcttctatgcctgaataggtgaccggag

SEQ ID NO: 252 - RNA, 28 nt, Picornavirus
caaugggguaccuucugggcauccuuca

SEQ ID NO: 253 - DNA, 147 bp, Picornavirus
  misc_feature (121)..(121): n is a, c, g, or t
tattcaacaaggggctgaaggatgcccagaaggtaccccattgtatgggatctgatctggggcctcggtgcacatgctttacatgtgtttagtcgaggttaaaaaacgtctaggccccccnaaccacggggacgtggttttcctttg

SEQ ID NO: 254 - RNA, 28 nt, Picornavirus
cccagcagggcagaaaacaucacauaau

SEQ ID NO: 255 - DNA, 124 bp, Picornavirus
tatcatgcctccccgattatgtgatgttttctgccctgctgggcggagcattctcgggttgagaaaccttgaatcttttcctttggaaccttggttcccccggtctaagccgcttggaatatga

SEQ ID NO: 256 - RNA, 28 nt, Picornavirus
uguguucuccgaaugugggauauccguc

SEQ ID NO: 257 - DNA, 121 bp, Picornavirus
cattcatgtcacctgcgagtgcttatcaatggttttatgacggatatcccacattcggagaacacaaacaggagaaagatcttgaatatggggcatgtcctaataacatgatgggcacttt

SEQ ID NO: 258 - RNA, 28 nt, Picornavirus
gcugcagaguugcccguuacgacagacu

SEQ ID NO: 259 - DNA, 95 bp, Picornavirus
atgcggctaatcctaactgcggagcagatacccacaaaccagtgggcagtctgtcgtaacgggcaactctgcagcggaaccgactactttgggtg

SEQ ID NO: 260 - RNA, 28 nt, Picornavirus
caauccaauucgcuuuaugauaacaauc

SEQ ID NO: 261 - DNA, 95 bp, Picornavirus
cgactactttgggtgtccgtgtttccttttattttataatggctgcttatggtgacaatcatagattgttatcataaagcgaattggattggcca

SEQ ID NO: 262 - RNA, 28 nt, Picornavirus
aauugucccgagccugguaaaagguaug

SEQ ID NO: 263 - DNA, 100 bp, Picornavirus
ctcaaggtgtcccaacataccttttaccaggctcgggacaattcctaacaactgatgatcatagctctgcaccagctctcccgtgtttcaacccaactcc

SEQ ID NO: 264 - RNA, 28 nt, Picornavirus
gcaacacuggauugugcgcacacgcucg

SEQ ID NO: 265 - DNA, 84 bp, Picornavirus
gctaatcccaacctccgagcgtgtgcgcacaatccagtgttgctacgtcgtaacgcgtaagttggaggcggaacagactacttt

SEQ ID NO: 266 - RNA, 28 nt, Picornavirus
acacccaaaguaguuggucccaucccgc

SEQ ID NO: 267 - DNA, 107 bp, Picornavirus
gcccctgaatgtggctaaccttaaccctgcagccagtgcacacaatccagtgtgtatctggtcgtaatgagcaattgcgggatgggaccaactactttgggtgtccg

SEQ ID NO: 268 - RNA, 28 nt, Picornavirus
uggauugugaugcaaggcuccgggguua

SEQ ID NO: 269 - DNA, 97 bp, Picornavirus
ccctgaatgcggctaaccttaaccccggagccttgcggcacaatccagtgttgttaaggtcgtaatgagcaattctgggatgggaccgactactttg

SEQ ID NO: 270 - RNA, 28 nt, Picornavirus
acauacaugcuggcuugcaugcaauagc

SEQ ID NO: 271 - DNA, 103 bp, Picornavirus
gcccctgaatgcggctaatcctaaccccgcagctattgcatgcaagccagcatgtatgtagtcgtaatgagcaattgtgggatggaaccgactactttgggtg

SEQ ID NO: 272 - RNA, 28 nt, Picornavirus
agccuaccccuuguggaagaucaaagag

SEQ ID NO: 273 - DNA, 114 bp, Picornavirus
gagtctaaattggggacgcagatgtttgggacgtcaccttgcagtgttaacttggctttcatgaacctctttgatcttccacaaggggtaggctacgggtgaaacctcttaggc

SEQ ID NO: 274 - RNA, 28 nt, Picornavirus
gcaaccacaucacugauuguucguacgu

SEQ ID NO: 275 - DNA, 129 bp, Picornavirus
cacgatctatgaagtcaccttcctcaagcgctggttcgttccggacgacgttaggcccatctacatccaccctgtgatggaccctgacacgtacgaacaatcagtgatgtggttgcgtgatggagattt

SEQ ID NO: 276 - RNA, 28 nt, Picornavirus
ccuuacaacuaguguuugcauuacuacc

SEQ ID NO: 277 - DNA, 135 bp, Picornavirus
  misc_feature (56)..(56): n is a, c, g, or t
ggccaaaagccaaggtttaacagaccctttaggattggttcaaacctgaaatgttntggaagatatttagtacctgctgatttggtagtagtgcaaacactagttgtaaggcccacgaaggatgcccagaaggta

SEQ ID NO: 278 - RNA, 28 nt, Respiratory syncytial virus
auuccacaaucaggagagucaugccugu

SEQ ID NO: 279 - DNA, 100 bp, Respiratory syncytial virus
agaggtggctccagaatacaggcatgactctcctgattgtggaatgataatattatgtatagcagcattagtaataaccaaattagcagcaggggataga

SEQ ID NO: 280 - RNA, 28 nt, Metapneumovirus
gcuugaguuauagcuugaucugccuccc

SEQ ID NO: 281 - DNA, 97 bp, Metapneumovirus
  misc_feature (65)..(65): n is a, c, g, or t
aagctgcaattagtggggaagcagatcaagctataactcaagctaggattgctccatacgctggnttgatcatgataatgacaatgaacaaccctaa

SEQ ID NO: 282 - RNA, 28 nt, Metapneumovirus
ucauaaucauuuugacugucgucacuca

SEQ ID NO: 283 - DNA, 137 bp, Metapneumovirus
aaaaagaggctgcagaacacttcctaaatgtgagtgacgacagtcaaaatgattatgagtaattaaaaaagtgggacaagtcaaaatgtcattccctgaaggaaaagatattcttttcatgggtaatgaagcagcaa

SEQ ID NO: 284 - RNA, 28 nt, Orthopneumovirus
gccuucgugaagcuuguucacguauguu

SEQ ID NO: 285 - DNA, 120 bp, Orthopneumovirus
tggggcaaatatggaaacatacgtgaacaaacttcacgaaggctccacatacacagctgctgttcaatacaatgtcctagaaaaagacgatgatcctgcatcacttacaatatgggtgcc

SEQ ID NO: 286 - RNA, 28 nt, Polyomavirus
uguaagcaaggcuuaaagguuguaucag

SEQ ID NO: 287 - DNA, 137 bp, Polyomavirus
ttatttggtgcttgcctgatacaacctttaagccttgcttacaagaagaaattaaaaactggaagcaaattttacagagtgaaatatcatatggtaaattttgtcaaatgatagaaaatgtagaagctggtcaggac

SEQ ID NO: 288 - RNA, 28 nt, Polyomavirus
uuggucacaugaaguacugggggaacau

SEQ ID NO: 289 - DNA, 96 bp, Polyomavirus
tcacaggaggggaaaatgttcccccagtacttcatgtgaccaacacagctaccacagtgttgctagatgaacagggtgtggggcctctttgtaaag

SEQ ID NO: 290 - RNA, 28 nt, Polyomavirus
ugccauacauaggcugcccaucaacucu

SEQ ID NO: 291 - DNA, 116 bp, Polyomavirus
aacagaaggacccctagagttgatgggcagcctatgtatggcatggatgctcaagtagaggaggttagagtttttgaggggacagaggaacttccaggggacccagacatgatgag

SEQ ID NO: 292 - RNA, 28 nt, Polyomavirus
uauagguaguugggccuuuauacuuguc

SEQ ID NO: 293 - DNA, 76 bp, Polyomavirus
ggtgtaacacccacagacaagtataaaggcccaactacctatacaattaatccaccaggagaccctagaacactgc

SEQ ID NO: 294 - RNA, 28 nt, Polyomavirus
agugaaacuuaauacuuuugcuccaccu

SEQ ID NO: 295 - DNA, 77 bp, Polyomavirus
caattagcagccacaaggtggagcaaaagtattaagtttcactgttatgtgcaggaatgtgcagctgtgacctttta

SEQ ID NO: 296 - RNA, 28 nt, Polyomavirus
caaaaagcuugagaaauggcauuaaaaa

SEQ ID NO: 297 - DNA, 77 bp, Polyomavirus
attggggtccaacactttttaatgccatttctcaagctttttggcgtgtaatacaaaatgacattcctaggctcacc

SEQ ID NO: 298 - RNA, 28 nt, Cowpox virus
gcuugaguuauagcuugaucugccuccc

SEQ ID NO: 299 - DNA, 125 bp, Cowpox virus
gctacgggcattgtcatctttaaaactctccactttccatcttctggagatcttctttcaatggtaggattataatatctgttgttataatcgtaatatccacaatcaggatctgtaaagcgagc

SEQ ID NO: 300 - RNA, 28 nt, Monkeypox virus
ucacgacgaggaucuauguaucuaacag

SEQ ID NO: 301 - DNA, 135 bp, Monkeypox virus
ccaccgcaatagatcctgttagatacatagatcctcgtcgtgatatcgcattttctaacgtgatggatatattaaagtcgaataaagttgaacaataattaattctttattgttatcatgaacggcggacatatt

SEQ ID NO: 302 - RNA, 28 nt, Vaccinia virus
aauccaucucagaauccgcugauggaaa

SEQ ID NO: 303 - DNA, 107 bp, Vaccinia virus
gacacgctggacaatctagcattcactgtgtttccatcagcggattctgagatggatttaatctgaggacatttggtgaatccaaagttcattctcagacctccacc

SEQ ID NO: 304 - RNA, 28 nt, Variola virus
aagaaucaaucaaaacuuaaucggucaa

SEQ ID NO: 305 - DNA, 108 bp, Variola virus
tggaccccaacatctttgaccgattaagttttgattgattcttccatgtaaggcgtatctagtcagatcgtataatctagccaacaatccatcgtcggtgtttaggtc

SEQ ID NO: 306 - RNA, 28 nt, Parapoxvirus
auggauccacccgaaaucacggccuaca

SEQ ID NO: 307 - DNA, 112 bp, Parapoxvirus
cggcaaccccgattatgtaggccgtgatttcgggtggatccatttagttattaaaattaatcatatacaactcttttatggcggctatggattcggctatccagtccttgac

SEQ ID NO: 308 - RNA, 28 nt, Reovirus
gcgugucguaguuugaguaguccagggc

SEQ ID NO: 309 - DNA, 121 bp, Reovirus
  misc_feature (14)..(14): n is a, c, g, or t
  misc_feature (23)..(23): n is a, c, g, or t
  misc_feature (32)..(32): n is a, c, g, or t
  misc_feature (50)..(50): n is a, c, g, or t
  misc_feature (62)..(62): n is a, c, g, or t
  misc_feature (104)..(104): n is a, c, g, or t
  misc_feature (107)..(107): n is a, c, g, or t
taatcggcgacctngaagcgacnggatcgcgngtgatggatgcggcaganaccttccgcaanaccggtgacgttgggatatggacattagccctggactactcnaantacgacacgcacat

SEQ ID NO: 310 - RNA, 28 nt, Reovirus
cgacagccaaauaugaaguacagcuuua

SEQ ID NO: 311 - DNA, 76 bp, Reovirus
ggactgccgaatacctaaagctgtacttcatatttggctgtcgaattccaaatctcagtcgtcatccaatcgtggg

SEQ ID NO: 312 - RNA, 28 nt, Reovirus
aucuaaucgaaaagcuggugaguggauc

SEQ ID NO: 313 - DNA, 99 bp, Reovirus
ttggaccatctgattctgcttcaaacgatccactcaccagcttttcgattagatcgaatgcagttaagacaaatgcagacgctggcgtgtctatggatt

SEQ ID NO: 314 - RNA, 28 nt, Reovirus
uagagcagcaauuucuuuugagcugugc

SEQ ID NO: 315 - DNA, 78 bp, Reovirus
atatcgtgtccttgagcacagctcaaaagaaattgctgctctacggattcacccaacctggtgtacagggtttgactg

SEQ ID NO: 316 - RNA, 28 nt, Reovirus
uuaaaucagguauaaaucuucuagcuga

SEQ ID NO: 317 - DNA, 91 bp, Reovirus
cacatgctgattacgtttcagctagaagatttatacctgatttaactgaactggttgatgctgaaaaacaaataaaagaaatggctgcaca

SEQ ID NO: 318 - RNA, 28 nt, Reovirus
caagugcgugauauccuccaccaguguu

SEQ ID NO: 319 - DNA, 145 bp, Reovirus
atctacttgcaccaggtggagcaacgaataacactggtggaggatatcacgcacttgttggaagagctactggaaagatggctgtcgtaactgcagttcaaggaagacccggaggaatcaattttgcacttgacatgaaagtacc

SEQ ID NO: 320 - RNA, 28 nt, Reovirus
aaaucuuuuguauugcucguuucuuacu

SEQ ID NO: 321 - DNA, 128 bp, Reovirus
cttgatttccagcaccagtgcactgatagtagtaagaaacgagcaatacaaaagatttgtgtcttaattagtaatgatcttagagagaatggactattagaagaggccaaaacattcaagccagagta

SEQ ID NO: 322 - RNA, 28 nt, Deltaretrovirus
guuaaaacaauaggcguuguccggaaag

SEQ ID NO: 323 - DNA, 97 bp, Deltaretrovirus
tgctaatacgcctccctttccggacaacgcctattgttttaacatcttgcctagttgataccaaaaacaactgggccatcataggtcgtgatgcctt

SEQ ID NO: 324 - RNA, 28 nt, Deltaretrovirus
ugaaggcgaaguauggcuggaacugcuu

SEQ ID NO: 325 - DNA, 98 bp, Deltaretrovirus
atagaccttactgacgcctttttccaaatccccctccccaagcagttccagccatacttcgccttcaccattccccagccatgtaattatggccccgg

SEQ ID NO: 326 - RNA, 28 nt, Human immunodeficiency virus
uuucuguuaaugcuuuuauuuuuucuuc

SEQ ID NO: 327 - DNA, 98 bp, Human immunodeficiency virus
aatggccattgacagaagaaaaaataaaagcattaacagaaatttgtacagaaatggaaaaggaaggaaaaatttcaaaaattgggcctgaaaatcca

SEQ ID NO: 328 - RNA, 28 nt, Human immunodeficiency virus
gucuagcagggaacacccaggcucuacc

SEQ ID NO: 329 - DNA, 82 bp, Human immunodeficiency virus
cggagaggctggcagattgagccctgggaggttctctccagcactagcaggtagagcctgggtgttccctgctagactctca

SEQ ID NO: 330 - RNA, 28 nt, Simian immunodeficiency virus
gcaacuaugauuauuuuucccucuagau

SEQ ID NO: 331 - DNA, 263 bp, Simian immunodeficiency virus
tggcaaatggattgtacccatctagagggaaaaataatcatagttgcagtacatgtagctagtggattcatagaagcagaagtaattccacaagaaacaggaagacagacagcactatttctgttaaaattggcaggcagatggcctattacacatctacacacagataatggtgctaactttacttcgcaagaagtaaagatggttgcatggtgggcagggatagagcacacctttggggtaccatacaatccacagagtca

SEQ ID NO: 332 - RNA, 28 nt, Rhabdovirus
auccaucauccucaucauugcuggcagc

SEQ ID NO: 333 - DNA, 75 bp, Rhabdovirus
ccaggattagactgggctgccagcaatgatgaggatgatggatctattgaggcagagattgcccatcagatagcc

SEQ ID NO: 334 - RNA, 28 nt, Rhabdovirus
cagggguucuugucccuccggaguaaag

SEQ ID NO: 335 - DNA, 81 bp, Rhabdovirus
tcagacgatgaggagctttactccggagggacaagaacccctgaagctgtgtacaccaggatcatggtcaatgggggaaag

SEQ ID NO: 336 - RNA, 28 nt, Rhabdovirus
gauugacaaagaucuugcucauguuugg

SEQ ID NO: 337 - DNA, 148 bp, Rhabdovirus
aacacccctccttttgaaccatcccaaacatgagcaagatctttgtcaatccgagtgctatcagagccggtctggctgatcttgagatggctgaagagactgttgatctgatcaatagaaacatagaagacaatcaggctcatctcca

SEQ ID NO: 338 - RNA, 28 nt, Rhabdovirus
cguccucuuggaacaacucauaauugga

SEQ ID NO: 339 - DNA, 89 bp, Rhabdovirus
caacgagctgaaaagtccaattatgagttgttccaagaggacggagtggaagagcatactaggccctcttattttcaggcagcagatga

SEQ ID NO: 340 - RNA, 28 nt, Rhabdovirus
gagccauuuugauaucuguuaaaaguuc

SEQ ID NO: 341 - DNA, 105 bp, Rhabdovirus
tatttggcctagagggaacttttaacagatatcaaaatggctcctacagttaagagaatcattaacgactccattattcagcctaagttaccggccaatgaggat

SEQ ID NO: 342 - RNA, 28 nt, Human smacovirus
ugaguauccaaaguacgacuuguuguca

SEQ ID NO: 343 - DNA, 120 bp, Human smacovirus
cctgaaccggtcttctgacaacaagtcgtattttggatactcatttgtaaaaacaaacactcttggactgtctatccacatttcttcccatgtgtacctgtcgtcccacatgtacccatt

SEQ ID NO: 344 - RNA, 28 nt, Chikungunya virus
uaccccgugguuuuuccauaaaggccug

SEQ ID NO: 345 - DNA, 91 bp, Chikungunya virus
gaataacgatgagcccaggcctttatggaaaaaccacggggtatgcggtaacccaccacgcagacggattcttgatgtgcaagactaccga

SEQ ID NO: 346 - RNA, 28 nt, Eastern equine encephalitis virus
caaugcgaugcacguaccgccuuuguuc

SEQ ID NO: 347 - DNA, 80 bp, Eastern equine encephalitis virus
agcagtggaccatttgaacaaaggcggtacgtgcatcgcattgggctatgggactgcggacagagccaccgagaacatta

SEQ ID NO: 348 - RNA, 28 nt, Togavirus
cuuacacaucaggaaacccucugcguga

SEQ ID NO: 349 - DNA, 90 bp, Togavirus
ttacgcagttacccatcacgcagagggtttcctgatgtgtaagatcactgatacagtcagaggagaaagagtctctttcccggtctgtac

SEQ ID NO: 350 - RNA, 28 nt, Togavirus
gugagugcaacagcgggugcugaaaaua

SEQ ID NO: 351 - DNA, 73 bp, Togavirus
acctggacagcggattattttcagcacccgctgttgcactcacctataaggatcatcactgggataattcgcc

SEQ ID NO: 352 - RNA, 28 nt, Togavirus
aagaagucggugcauggacugcauagac

SEQ ID NO: 353 - DNA, 80 bp, Togavirus
cagaggtggcagtctatcaggatgtctatgcagttcatgcaccgacttctttgtacttccaggcaatgaaaggagtacgc

SEQ ID NO: 354 - RNA, 28 nt, Togavirus
gccacucucucagcagucauagcguacc

SEQ ID NO: 355 - DNA, 122 bp, Togavirus
ttccgtgtctgtgtaggtacgctatgactgctgagagagtggcaagacttcggatgaacaacactaaggccataattgtgtgctcctccttccctttaccgaagtacaggattgaaggcgtc

SEQ ID NO: 356 - RNA, 28 nt, Togavirus
ugaugguacagcgauguuggugcaugua

SEQ ID NO: 357 - DNA, 78 bp, Togavirus
aggacgtgtatgctgtacatgcaccaacatcgctgtaccatcaggcgatgaaaggtgtcagaacggcgtattggattg

SEQ ID NO: 358 - RNA, 28 nt, Togavirus
uccgucgaaaauauguacccaccuaccc

SEQ ID NO: 359 - DNA, 82 bp, Togavirus
aatactgactaaccggggtaggtgggtacatattttcgacggacacaggccctgggcacttgcaaaagaagtccgttctgca

SEQ ID NO: 360 - RNA, 28 nt, Togavirus
cuggcguuagcauggucguuauccguga

SEQ ID NO: 361 - DNA, 129 bp, Togavirus
tttgaggtagaagccaagcaggtcactgataatgaccatgctaacgccagagcgttttcgcatctggcttcaaaattgatcgaaacggaggtggacccatccgacacgatccttgacattggaagtgcg

SEQ ID NO: 362 - RNA, 28 nt, Togavirus
cagugaacaggugaugcaaugauugcau

SEQ ID NO: 363 - DNA, 124 bp, Togavirus
ggcaaagatcgagtgatgcaatcattgcatcacctgttcactgctttcgacactacggatgccgatgtcaccatatattgcttggataaacaatgggagaccaggataatcgaggccattcacc

SEQ ID NO: 364 - RNA, 28 nt, Togavirus
gccccacucgauccaauggcggcgggua

SEQ ID NO: 365 - DNA, 73 bp, Togavirus
cgcaatttcgcggtatacccgccgccattggatcgagtggggccctaaagaagccctacacgtcctcatcgac

SEQ ID NO: 366 - DNA, 40 bp, Coronavirus
gttaatacgactcactatagggctttgctgagttggaagc

SEQ ID NO: 367 - DNA, 18 bp, Coronavirus
agaacttgtggtgaggtg

SEQ ID NO: 368 - DNA, 15 bp, Ebolavirus
ccaggttaggaggca

SEQ ID NO: 369 - DNA, 40 bp, Zaire ebolavirus
gttaatacgactcactataggggcctaacagatcgaccaa

SEQ ID NO: 370 - DNA, 18 bp, Ebolavirus
tctgtctgccctctgtat

SEQ ID NO: 371 - DNA, 40 bp, Dengue virus
gttaatacgactcactatagggacgcctttcaatatgctg

SEQ ID NO: 372 - DNA, 20 bp, Dengue virus
tgagaatctctttgtcagct

SEQ ID NO: 373 - DNA, 41 bp, Dengue virus
gttaatacgactcactatagggccgtctttcaatatgctga

SEQ ID NO: 374 - DNA, 18 bp, Dengue virus
tgagaatctcttcgccaa

SEQ ID NO: 375 - DNA, 37 bp, Zika virus
gttaatacgactcactatagggaccccatgtggagag

SEQ ID NO: 376 - DNA, 18 bp, Zika virus
ttccttcagtgtgtcacc

SEQ ID NO: 377 - DNA, 37 bp, Herpes simplex virus
gttaatacgactcactatagggcgtacacctcgaacg

SEQ ID NO: 378 - DNA, 18 bp, Herpes simplex virus
accatcgagctgtacaag

SEQ ID NO: 379 - DNA, 40 bp, Alphainfluenzavirus
gttaatacgactcactatagggtctaatgtcgcagtctcg

SEQ ID NO: 380 - DNA, 20 bp, Alphainfluenzavirus
tcattgccatcatccatttc

SEQ ID NO: 381 - DNA, 40 bp, Measles virus
gttaatacgactcactatagggacagctgctgaaggaatt

SEQ ID NO: 382 - DNA, 18 bp, Measles virus
ctagccggctggatttta

SEQ ID NO: 383 - DNA, 40 bp, Mumps virus
gttaatacgactcactatagggatgctcacctatcactgc

SEQ ID NO: 384 - DNA, 18 bp, Mumps virus
aatctcgttcgcactgat

SEQ ID NO: 385 - DNA, 40 bp, Human immunodeficiency virus
gttaatacgactcactatagggatggccattgacagaaga

SEQ ID NO: 386 - DNA, 18 bp, Human immunodeficiency virus
tggattttcaggcccaat

SEQ ID NO: 387 - DNA, 40 bp, Rabies virus
gttaatacgactcactatagggacacccctccttttgaac

SEQ ID NO: 388 - DNA, 18 bp, Rabies virus
tggagatgagcctgattg

SEQ ID NO: 389 - DNA, 40 bp, Chikungunya virus
gttaatacgactcactatagggaataacgatgagcccagg

SEQ ID NO: 390 - DNA, 18 bp, Chikungunya virus
tcggtagtcttgcacatc

SEQ ID NO: 391 - DNA, 39 bp, Mammarenavirus
gttaatacgactcactatagggatggcactcacaacagg

SEQ ID NO: 392 - DNA, 16 bp, Mammarenavirus
ggatcatgtcagcacc

SEQ ID NO: 393 - DNA, 15 bp, Mammarenavirus
gaccatgtaagcacc

SEQ ID NO: 394 - DNA, 17 bp, Mammarenavirus
gggatcatgttagcact

SEQ ID NO: 395 - DNA, 40 bp, Mammarenavirus
gttaatacgactcactatagggtcagtgcattgacgacag

SEQ ID NO: 396 - DNA, 18 bp, Mammarenavirus
ggaaggatcatgtcagca

SEQ ID NO: 397 - DNA, 41 bp, Mammarenavirus
gttaatacgactcactatagggtcattgcattcacaacagg

SEQ ID NO: 398 - DNA, 19 bp, Mammarenavirus
aggtgtatgatgttggtga

SEQ ID NO: 399 - DNA, 40 bp, Mammarenavirus
gttaatacgactcactatagggcatcgcacttacaacagg

SEQ ID NO: 400 - DNA, 20 bp, Mammarenavirus
aagtgtatgatgttggtgat

SEQ ID NO: 401 - DNA, 17 bp, Mammarenavirus
gggatcatgttagcacc

SEQ ID NO: 402 - DNA, 40 bp, Norwalk virus
gttaatacgactcactatagggagccaatgttcagatgga

SEQ ID NO: 403 - DNA, 18 bp, Norwalk virus
attcgacgccatcttcat

SEQ ID NO: 404 - DNA, 38 bp, Norwalk virus
gttaatacgactcactatagggccatgttccgctggat

SEQ ID NO: 405 - DNA, 39 bp, Norwalk virus
gttaatacgactcactataggggatctgttctgcgctgg

SEQ ID NO: 406 - DNA, 40 bp, Norwalk virus
gttaatacgactcactatagggacccatgttcaggtggat

SEQ ID NO: 407 - DNA, 40 bp, Papillomavirus
gttaatacgactcactatagggacaaggctttggaaccaa

SEQ ID NO: 408 - DNA, 15 bp, Papillomavirus
ttgcagtgcattgcg

SEQ ID NO: 409 - DNA, 37 bp, Papillomavirus
gttaatacgactcactatagggtaggctgtggacaca

SEQ ID NO: 410 - DNA, 15 bp, Papillomavirus
ttgtagtgcactgcg

SEQ ID NO: 411 - DNA, 37 bp, Papillomavirus
gttaatacgactcactatagggaggctttggacacaa

SEQ ID NO: 412 - DNA, 15 bp, Papillomavirus
cttgcagtgcattgc

SEQ ID NO: 413 - DNA, 37 bp, Papillomavirus
gttaatacgactcactatagggtgggctttggagaca

SEQ ID NO: 414 - DNA, 37 bp, Phlebovirus
gttaatacgactcactataggggatcctggtgtctgg

SEQ ID NO: 415 - DNA, 18 bp, Phlebovirus
cctttcccaacatgctgt

SEQ ID NO: 416 - DNA, 37 bp, Phlebovirus; identical to SEQ ID NO: 414

SEQ ID NO: 417 - DNA, 18 bp, Phlebovirus
cctttacctacatgctgc

SEQ ID NO: 418 - DNA, 18 bp, Phlebovirus
gccctttccctacatgtt

SEQ ID NO: 419 - DNA, 36 bp, Phlebovirus
gttaatacgactcactataggggctcttggtgcctg

SEQ ID NO: 420 - DNA, 16 bp, Phlebovirus
ctgggcccacatgttg

SEQ ID NO: 421 - DNA, 16 bp, Phlebovirus
ggcacccacatgttgt

SEQ ID NO: 422 - DNA, 16 bp, Phlebovirus
ggcacccacatgttgt

SEQ ID NO: 423 - DNA, 40 bp, Phlebovirus
gttaatacgactcactataggggttcatggtgtcagatgg

SEQ ID NO: 424 - DNA, 17 bp, Phlebovirus
ctttccccacatgctgt

SEQ ID NO: 425 - DNA, 40 bp, Phlebovirus
gttaatacgactcactataggggatcttggtgccagatgg

SEQ ID NO: 426 - DNA, 20 bp, Sapporo virus
ggdcthccmtcwggsatgcc

SEQ ID NO: 427 - DNA, 20 bp, Sapporo virus
tahabrcartcatcmccrta

SEQ ID NO: 428 - DNA, 17 bp, Simian immunodeficiency virus
tggctggaytgtacmca

SEQ ID NO: 429 - DNA, 20 bp, Simian immunodeficiency virus
tgwctytgtggattrtawgg

SEQ ID NO: 430 - DNA, 39 bp, Hepatitis delta virus
gttaatacgactcactatagggccggctactcttcttgc

SEQ ID NO: 431 - DNA, 16 bp, Hepatitis delta virus
caccgacgaaggaagg

SEQ ID NO: 432 - DNA, 40 bp, Hepatitis delta virus
gttaatacgactcactatagggccggctactcttctttcc

SEQ ID NO: 433 - DNA, 17 bp, Hepatitis delta virus
ccaccgaagaaggaagg

SEQ ID NO: 434 - DNA, 40 bp, Hepatitis delta virus
gttaatacgactcactatagggccggctgttcttcttttc

SEQ ID NO: 435 - DNA, 18 bp, Hepatitis delta virus
ttcgacgaacagaagacc

SEQ ID NO: 436 - DNA, 40 bp, Mastadenovirus
gttaatacgactcactatagggatggattcgggggagtat

SEQ ID NO: 437 - DNA, 18 bp, Mastadenovirus
tgtttttgaccccgatga

SEQ ID NO: 438 - DNA, 37 bp, Mastadenovirus
gttaatacgactcactatagggtaggtgacgagacgc

SEQ ID NO: 439 - DNA, 15 bp, Mastadenovirus
tttacagccagcacg

SEQ ID NO: 440 - DNA, 40 bp, Mastadenovirus
gttaatacgactcactatagggtgcgttctcttccttgtt

SEQ ID NO: 441 - DNA, 18 bp, Mastadenovirus
gtaggagccatataccgc

SEQ ID NO: 442 - DNA, 40 bp, Mastadenovirus
gttaatacgactcactatagggcctggcctacaactatgg

SEQ ID NO: 443 - DNA, 18 bp, Mastadenovirus
gaccagtagacttgctcc

SEQ ID NO: 444 - DNA, 40 bp, Mastadenovirus
gttaatacgactcactatagggcagcgcttggattacatg

SEQ ID NO: 445 - DNA, 18 bp, Mastadenovirus
gtgtgtacctttggtgga

SEQ ID NO: 446 - DNA, 37 bp, Torque teno virus
gttaatacgactcactataggggaacttgggcgggtg

SEQ ID NO: 447 - DNA, 17 bp, Torque teno virus
cgccagactgatctagc

SEQ ID NO: 448 - DNA, 38 bp, Torque teno virus
gttaatacgactcactatagggtgatcttgggcgggag

SEQ ID NO: 449 - DNA, 18 bp, Torque teno virus
caccagactgaactagcc

SEQ ID NO: 450 - DNA, 40 bp, Avian gyrovirus
gttaatacgactcactatagggtatgcgcgtagaagatcc

SEQ ID NO: 451 - DNA, 18 bp, Avian gyrovirus
gcctccggaatgaataca

SEQ ID NO: 452 - DNA, 40 bp, Chicken anemia virus
gttaatacgactcactataggggaacgctctccaagaaga

SEQ ID NO: 453 - DNA, 18 bp, Chicken anemia virus
ttccagcgataccaatcc

SEQ ID NO: 454 - DNA, 40 bp, Torque teno virus
gttaatacgactcactataggggctcaagtcctcatttgc

SEQ ID NO: 455 - DNA, 15 bp, Torque teno virus
ctcagccattcggaa

SEQ ID NO: 456 - DNA, 40 bp, Torque teno virus
gttaatacgactcactatagggagctccggtcatacaatg

SEQ ID NO: 457 - DNA, 17 bp, Torque teno virus
gtacggaaccagtgtcc

SEQ ID NO: 458 - DNA, 44 bp, Torque teno virus
gttaatacgactcactataggggctwcagtaagatattacccct

SEQ ID NO: 459 - DNA, 16 bp, Torque teno virus
gytcccaacctckaac

SEQ ID NO: 460 - DNA, 40 bp, Torque teno virus
gttaatacgactcactataggggagtttttgctgctggag

SEQ ID NO: 461 - DNA, 15 bp, Mammarenavirus
tcatgggtgaggcac

SEQ ID NO: 462 - DNA, 34 bp, Mammarenavirus
gttaatacgactcactataggggggcggtgggtc

SEQ ID NO: 463 - DNA, 20 bp, Mammarenavirus
ataatgtatgatgcagctgt

SEQ ID NO: 464 - DNA, 38 bp, Mammarenavirus
gttaatacgactcactatagggctattggcggtgggtc

SEQ ID NO: 465 - DNA, 18 bp, Mammarenavirus
catgtttgatgcagcagt

SEQ ID NO: 466 - DNA, 40 bp, Mammarenavirus
gttaatacgactcactatagggtgacaattgtgtgggtgt

SEQ ID NO: 467 - DNA, 16 bp, Mammarenavirus
gtcatgggtgaagcac

SEQ ID NO: 468 - DNA, 37 bp, Mammarenavirus
gttaatacgactcactatagggatgctccctcttcca

SEQ ID NO: 469 - DNA, 18 bp, Mammarenavirus
ccatggtctttactgcac

SEQ ID NO: 470 - DNA, 37 bp, Mammarenavirus
gttaatacgactcactatagggggtgctctctcttcc

SEQ ID NO: 471 - DNA, 19 bp, Mammarenavirus
tcaatggttttcactgcac

SEQ ID NO: 472 - DNA, 40 bp, Mamastrovirus
gttaatacgactcactatagggtccatgggaagctcctat

SEQ ID NO: 473 - DNA, 17 bp, Mamastrovirus
gagtcacgaagctgctt

SEQ ID NO: 474 - DNA, 37 bp, Coronavirus
gttaatacgactcactatagggagtgtccgtgatggt

SEQ ID NO: 475 - DNA, 18 bp, Coronavirus
gctctaccgctaacactt

SEQ ID NO: 476 - DNA, 40 bp, Coronavirus
gttaatacgactcactatagggtggtgaatggaatgctgt

SEQ ID NO: 477 - DNA, 18 bp, Coronavirus
caccaacactccaactct

SEQ ID NO: 478 - DNA, 40 bp, Coronavirus
gttaatacgactcactataggggaagtcagatgagggtgg

SEQ ID NO: 479 - DNA, 18 bp, Coronavirus
acatgccattcttgtcca

SEQ ID NO: 480 - DNA, 40 bp, Coronavirus
gttaatacgactcactataggggtctgcatgttgttggac

SEQ ID NO: 481 - DNA, 18 bp, Coronavirus
ctgctgacaacaatggtg

SEQ ID NO: 482 - DNA, 40 bp, Reston Ebolavirus
gttaatacgactcactatagggaattcagttgctcaggct

SEQ ID NO: 483 - DNA, 18 bp, Reston Ebolavirus
gtcttactccttggtcgg

SEQ ID NO: 484 - DNA, 40 bp, Marburgvirus
gttaatacgactcactatagggttcatcaactgagggtcg

SEQ ID NO: 485 - DNA, 18 bp, Marburgvirus
tactgagaacatgtcggc

SEQ ID NO: 486 - DNA, 40 bp, Bagazavirus
gttaatacgactcactatagggtctggatctgatggacca

SEQ ID NO: 487 - DNA, 18 bp, Bagazavirus
ttgtccccgatgatgatg

SEQ ID NO: 488 - DNA, 40 bp, Culex flavivirus
gttaatacgactcactataggggctgtgggaatcgacata

SEQ ID NO: 489 - DNA, 18 bp, Culex flavivirus
agttcagcagtaccatcg

SEQ ID NO: 490 - DNA, 37 bp, Japanese encephalitis virus
gttaatacgactcactatagggtgtggaagaccgcat

SEQ ID NO: 491 - DNA, 18 bp, Japanese encephalitis virus
actcctggttttgtctgg

SEQ ID NO: 492 - DNA, 40 bp, Kyasanur Forest disease virus
gttaatacgactcactatagggtccagtgcatgctcatag

SEQ ID NO: 493 - DNA, 15 bp, Kyasanur Forest disease virus
ccacacaactgcaca

SEQ ID NO: 494 - DNA, 37 bp, Murray Valley encephalitis virus
gttaatacgactcactatagggaatatgctacgcggc

SEQ ID NO: 495 - DNA, 15 bp, Murray Valley encephalitis virus
gcaagtgctgtcctg

SEQ ID NO: 496 - DNA, 40 bp, Powassan virus
gttaatacgactcactatagggttggggcaagtcaatctt

SEQ ID NO: 497 - DNA, 18 bp, Powassan virus
aacactcctgttgctctc

SEQ ID NO: 498 - DNA, 40 bp, Saint Louis encephalitis virus
gttaatacgactcactatagggcggggttgaagaggatac

SEQ ID NO: 499 - DNA, 18 bp, Saint Louis encephalitis virus
atctacagccctccatct

SEQ ID NO: 500 - DNA, 40 bp, Tembusu virus
gttaatacgactcactatagggagggagtgaatggtgttg

SEQ ID NO: 501 - DNA, 18 bp, Tembusu virus
aattccgtagcctccatg

SEQ ID NO: 502 - DNA, 40 bp, Tick-borne encephalitis virus
gttaatacgactcactatagggagaacaagagctggggat

SEQ ID NO: 503 - DNA, 18 bp, Tick-borne encephalitis virus
cggtctctttcgacactc

SEQ ID NO: 504 - DNA, 40 bp, Usutu virus
gttaatacgactcactatagggtgtctccaactgtccaac

SEQ ID NO: 505 - DNA, 18 bp, Usutu virus
tggcacacgtgtctatac

SEQ ID NO: 506 - DNA, 40 bp, West Nile virus
gttaatacgactcactatagggaagtctggaagcagcatt

SEQ ID NO: 507 - DNA, 18 bp, West Nile virus
ccaagctgtgtctcctag

SEQ ID NO: 508 - DNA, 37 bp, Yellow fever virus
gttaatacgactcactatagggttggtctgctcgagt

SEQ ID NO: 509 - DNA, 18 bp, Yellow fever virus
gtaccatattgacgccca

SEQ ID NO: 510 - DNA, 38 bp, Hepatitis C virus
gttaatacgactcactatagggtgagcacacttcctcc

SEQ ID NO: 511 - DNA, 15 bp, Hepatitis C virus
gcgcggcaacaagta

SEQ ID NO: 512 - DNA, 38 bp, Pegivirus
gttaatacgactcactataggggtacgggttggagcct

SEQ ID NO: 513 - DNA, 17 bp, Pegivirus
ggcttctccgatgtcag

SEQ ID NO: 514 - DNA, 41 bp, Pegivirus
gttaatacgactcactatagggggtatggaatggaacctga

SEQ ID NO: 515 - DNA, 17 bp, Pegivirus
ggcttcaccaatgtcag

SEQ ID NO: 516 - DNA, 36 bp, Pegivirus
gttaatacgactcactatagggatgtcagctgggca

SEQ ID NO: 517 - DNA, 16 bp, Pegivirus
cattctgggtcgtcgg

SEQ ID NO: 518 - DNA, 37 bp, Pegivirus
gttaatacgactcactatagggtgttagctgggcaac

SEQ ID NO: 519 - DNA, 16 bp, Pegivirus
cattgggggtcatccg

SEQ ID NO: 520 - DNA, 40 bp, Pegivirus
gttaatacgactcactataggggtggccatcaagctatct

SEQ ID NO: 521 - DNA, 18 bp, Pegivirus
aactccaccaaccaagag

SEQ ID NO: 522 - DNA, 37 bp, Hantavirus
gttaatacgactcactatagggtggctacaccagttg

SEQ ID NO: 523 - DNA, 18 bp, Hantavirus
catccaggacattcccat

SEQ ID NO: 524 - DNA, 40 bp, Hantavirus
gttaatacgactcactatagggctttccagttgggtcact

SEQ ID NO: 525 - DNA, 18 bp, Hantavirus
tctgaccagtcatgcttt

SEQ ID NO: 526 - DNA, 40 bp, Hantavirus
gttaatacgactcactatagggcacaatggcccagtagaa

SEQ ID NO: 527 - DNA, 18 bp, Hantavirus
acatggcttctagtgcag

SEQ ID NO: 528 - DNA, 40 bp, Hantavirus
gttaatacgactcactatagggggcacaataggagcagta

SEQ ID NO: 529 - DNA, 18 bp, Hantavirus
caattaggtcatggcgga

SEQ ID NO: 530 - DNA, 40 bp, Hantavirus
gttaatacgactcactatagggagagcactaatcacagca

SEQ ID NO: 531 - DNA, 17 bp, Hantavirus
gcagcttcctttgcttc

SEQ ID NO: 532 - DNA, 40 bp, Hantavirus; identical to SEQ ID NO: 530

SEQ ID NO: 533 - DNA, 16 bp, Hantavirus
cagcctcctttgcctc

SEQ ID NO: 534 - DNA, 40 bp, Hantavirus
gttaatacgactcactatagggagaggatataacccgcca

SEQ ID NO: 535 - DNA, 18 bp, Hantavirus
ctgacactgtttgttgcc

SEQ ID NO: 536 - DNA, 37 bp, Hantavirus
gttaatacgactcactatagggcacgtctcaggtggt

SEQ ID NO: 537 - DNA, 18 bp, Hantavirus
cttgtacttggcctgaca

SEQ ID NO: 538 - DNA, 40 bp, Hantavirus
gttaatacgactcactatagggacattacagagcagacgg

SEQ ID NO: 539 - DNA, 18 bp, Hantavirus
aggttcaatccctgttgg

SEQ ID NO: 540 - DNA, 37 bp, Hantavirus
gttaatacgactcactatagggaaccctgagaaggca

SEQ ID NO: 541 - DNA, 18 bp, Hantavirus
tagactgctgctgaatgg

SEQ ID NO: 542 - DNA, 40 bp, Hantavirus
gttaatacgactcactatagggcgacccggatgatgttaa

SEQ ID NO: 543 - DNA, 18 bp, Hantavirus
acaggcttttcacccatt

SEQ ID NO: 544 - DNA, 40 bp, Hepatitis B virus
gttaatacgactcactatagggcacctgtattcccatccc

SEQ ID NO: 545 - DNA, 15 bp, Hepatitis B virus
aactgagccaggagc

SEQ ID NO: 546 - DNA, 37 bp, Orthohepevirus
gttaatacgactcactatagggtgcctatgctgcccg

SEQ ID NO: 547 - DNA, 17 bp, Orthohepevirus
gcgaagggctgagaatc

SEQ ID NO: 548 - DNA, 40 bp, Cytomegalovirus
gttaatacgactcactatagggaagaggtttcaagtgcga

SEQ ID NO: 549 - DNA, 18 bp, Cytomegalovirus
tcttggaccacagttgtc

SEQ ID NO: 550 - DNA, 40 bp, Lymphocryptovirus
gttaatacgactcactatagggtgtctgtggttgtcttcc

SEQ ID NO: 551 - DNA, 18 bp, Lymphocryptovirus
gaactgcgggataatgga

SEQ ID NO: 552 - DNA, 40 bp, Rhadinovirus
gttaatacgactcactatagggagccattatacacacggg

SEQ ID NO: 553 - DNA, 18 bp, Rhadinovirus
gggaagttgtgtgtcaga

SEQ ID NO: 554 - DNA, 37 bp, Herpes simplex virus
gttaatacgactcactatagggtgaaggcagagacgt

SEQ ID NO: 555 - DNA, 18 bp, Herpes simplex virus
gagttgctcctggagtac

SEQ ID NO: 556 - DNA, 40 bp, Varicellovirus
gttaatacgactcactatagggtccttggttggttttggt

SEQ ID NO: 557 - DNA, 18 bp, Varicellovirus
tacattcggattctggcc

SEQ ID NO: 558 - DNA, 40 bp, Crimean-Congo hemorrhagic fever virus
gttaatacgactcactatagggctgaatctgtggaggcag

SEQ ID NO: 559 - DNA, 18 bp, Crimean-Congo hemorrhagic fever virus
cgctctattgaatgcacc

SEQ ID NO: 560 - DNA, 40 bp, Orthonairovirus
gttaatacgactcactatagggccttgaactagccaagca

SEQ ID NO: 561 - DNA, 15 bp, Orthonairovirus
ctgtgagactgtcgg

SEQ ID NO: 562 - DNA, 40 bp, Orthomyxovirus
gttaatacgactcactatagggcaggcagcaatttcaaca

SEQ ID NO: 563 - DNA, 18 bp, Orthomyxovirus
gttctgatcacggtgtct

SEQ ID NO: 564 - DNA, 40 bp, Orthomyxovirus
gttaatacgactcactatagggtctgctttaggaggacca

SEQ ID NO: 565 - DNA, 18 bp, Orthomyxovirus
ttgtactgctctgacacc

SEQ ID NO: 566 - DNA, 40 bp, Papillomavirus
gttaatacgactcactatagggagtgggtatggcaatacg

SEQ ID NO: 567 - DNA, 18 bp, Papillomavirus
gttagatctgcctctccg

SEQ ID NO: 568 - DNA, 41 bp, Papillomavirus
gttaatacgactcactatagggtccagattagatttgcacg

SEQ ID NO: 569 - DNA, 16 bp, Papillomavirus
acacatttcgttggga

SEQ ID NO: 570 - DNA, 40 bp, Papillomavirus
gttaatacgactcactataggggcagattagacttgcagc

SEQ ID NO: 571 - DNA, 14 bp, Papillomavirus
cgcacttcgttccg

SEQ ID NO: 572 - DNA, 40 bp, Papillomavirus
gttaatacgactcactatagggtacagacctacgtgacca

SEQ ID NO: 573 - DNA, 18 bp, Papillomavirus
aatcccatttctctggcc

SEQ ID NO: 574 - DNA, 40 bp, Paramyxovirus
gttaatacgactcactatagggggggcatctatcaagcat

SEQ ID NO: 575 - DNA, 18 bp, Paramyxovirus
gctctgggttaatgtcga

SEQ ID NO: 576 - DNA, 37 bp, Paramyxovirus
gttaatacgactcactatagggagaggcaacagctgt

SEQ ID NO: 577 - DNA, 18 bp, Paramyxovirus
accaggatagagtcagca

SEQ ID NO: 578 - DNA, 38 bp, Papillomavirus
gttaatacgactcactatagggtgaacttactgaccgc

SEQ ID NO: 579 - DNA, 14 bp, Papillomavirus
cactgcgctcgttg

SEQ ID NO: 580 - DNA, 38 bp, Papillomavirus
gttaatacgactcactatagggtgagttaactgaccgc

SEQ ID NO: 581 - DNA, 15 bp, Papillomavirus
tcgcgttttgtcagc

SEQ ID NO: 582 - DNA, 38 bp, Papillomavirus
gttaatacgactcactatagggcgaactaactgaccgc

SEQ ID NO: 583 - DNA, 14 bp, Papillomavirus
attgcgctcgctga

SEQ ID NO: 584 - DNA, 40 bp, Paramyxovirus
gttaatacgactcactataggggagtcacaaccatcagct

SEQ ID NO: 585 - DNA, 19 bp, Paramyxovirus
tgtgataatgcctccatca

SEQ ID NO: 586 - DNA, 40 bp, Paramyxovirus
gttaatacgactcactatagggtgtcaccacaatcagctg

SEQ ID NO: 587 - DNA, 18 bp, Paramyxovirus
gtgatatcgcctccatca

SEQ ID NO: 588 - DNA, 40 bp, Paramyxovirus
gttaatacgactcactatagggaaggaactccaacaccag

SEQ ID NO: 589 - DNA, 15 bp, Paramyxovirus
tggggtggaagttgt

SEQ ID NO: 590 - DNA, 37 bp, Paramyxovirus
gttaatacgactcactatagggatcgtgagggggaag

SEQ ID NO: 591 - DNA, 18 bp, Paramyxovirus
gtgaacactgacgacatc

SEQ ID NO: 592 - DNA, 40 bp, Paramyxovirus
gttaatacgactcactatagggactactcccgaggacaat

SEQ ID NO: 593 - DNA, 18 bp, Paramyxovirus
ctgcgtacatcaggagtt

SEQ ID NO: 594 - DNA, 37 bp, Paramyxovirus
gttaatacgactcactatagggttttgcccctggagg

SEQ ID NO: 595 - DNA, 18 bp, Paramyxovirus
ggctcaagataaccacga

SEQ ID NO: 596 - DNA, 40 bp, Paramyxovirus
gttaatacgactcactatagggagctggtaatcctggaga

SEQ ID NO: 597 - DNA, 15 bp, Paramyxovirus
tggtgggttctctcc

SEQ ID NO: 598 - DNA, 40 bp, Paramyxovirus
gttaatacgactcactatagggacgtgggcaactttagaa

SEQ ID NO: 599 - DNA, 15 bp, Paramyxovirus
ctcccagggcaacta

SEQ ID NO: 600 - DNA, 40 bp, Paramyxovirus
gttaatacgactcactataggggaggacacagaagagagc

SEQ ID NO: 601 - DNA, 19 bp, Paramyxovirus
tgcagattggattacacca

SEQ ID NO: 602 - DNA, 40 bp, Paramyxovirus
gttaatacgactcactatagggtgcagggataggaggaat

SEQ ID NO: 603 - DNA, 18 bp, Paramyxovirus
atccactgtgaaggttgg

SEQ ID NO: 604 - DNA, 40 bp, Paramyxovirus
gttaatacgactcactatagggtgaagaccttgtccacac

SEQ ID NO: 605 - DNA, 18 bp, Paramyxovirus
accctgagatgctagtga

SEQ ID NO: 606 - DNA, 40 bp, Paramyxovirus
gttaatacgactcactatagggggaggaggtgctgttatc

SEQ ID NO: 607 - DNA, 18 bp, Paramyxovirus
ctaggaaggtggttgcaa

SEQ ID NO: 608 - DNA, 40 bp, Paramyxovirus
gttaatacgactcactatagggcaagttcacctgcacatg

SEQ ID NO: 609 - DNA, 18 bp, Paramyxovirus
gtctgaaggcgaagatca

SEQ ID NO: 610 - DNA, 40 bp, Paramyxovirus
gttaatacgactcactatagggcatgggagttggaagtgt

SEQ ID NO: 611 - DNA, 18 bp, Paramyxovirus
cctggtgtttcattgcag

SEQ ID NO: 612 - DNA, 40 bp, Paramyxovirus
gttaatacgactcactatagggggcccaagatgctatcat

SEQ ID NO: 613 - DNA, 18 bp, Paramyxovirus
ctccccagtaggatcctt

SEQ ID NO: 614 - DNA, 37 bp, Parvovirus
gttaatacgactcactatagggaactcagtggcagct

SEQ ID NO: 615 - DNA, 18 bp, Parvovirus
gctacaacttcggaggaa

SEQ ID NO: 616 - DNA, 40 bp, Peribunyavirus
gttaatacgactcactatagggataagacgccacaaccaa

SEQ ID NO: 617 - DNA, 18 bp, Peribunyavirus
tgacactggatttgcagt

SEQ ID NO: 618 - DNA, 40 bp, Peribunyavirus
gttaatacgactcactatagggtaagcgtatccacaccac

SEQ ID NO: 619 - DNA, 18 bp, Peribunyavirus
ccccaaggttaagcgtaa

SEQ ID NO: 620 - DNA, 40 bp, Peribunyavirus
gttaatacgactcactatagggaatttggagagtggcagg

SEQ ID NO: 621 - DNA, 19 bp, Peribunyavirus
tggatggtaagatcgttgt

SEQ ID NO: 622 - DNA, 40 bp, Peribunyavirus
gttaatacgactcactatagggagtccagtcctcgatgat

SEQ ID NO: 623 - DNA, 18 bp, Peribunyavirus
cttgctcaggtgctgata

SEQ ID NO: 624 - DNA, 40 bp, Peribunyavirus
gttaatacgactcactataggggatgtaccacaacggact

SEQ ID NO: 625 - DNA, 18 bp, Peribunyavirus
tgagcacttgtccgtatc

SEQ ID NO: 626 - DNA, 40 bp, Peribunyavirus
gttaatacgactcactataggggctgatcttctcatggct

SEQ ID NO: 627 - DNA, 15 bp, Peribunyavirus
gcgaatgttggcagt

SEQ ID NO: 628 - DNA, 40 bp, Peribunyavirus
gttaatacgactcactatagggtctcgctacgtttaaccc

SEQ ID NO: 629 - DNA, 18 bp, Peribunyavirus
gccgtcttactgagtacc

SEQ ID NO: 630 - DNA, 40 bp, Phlebovirus
gttaatacgactcactatagggggagacaatagccaggtc

SEQ ID NO: 631 - DNA, 18 bp, Phlebovirus
gatgttgcacaagtccac

SEQ ID NO: 632 - DNA, 40 bp, Phlebovirus
gttaatacgactcactatagggtgaatcatgcaagggtgt

SEQ ID NO: 633 - DNA, 19 bp, Phlebovirus
gcactatgcctccttagaa

SEQ ID NO: 634 - DNA, 37 bp, Phlebovirus
gttaatacgactcactatagggtgagtcatgcggtgt

SEQ ID NO: 635 - DNA, 18 bp, Phlebovirus
gcactatgccttcgtaga

SEQ ID NO: 636 - DNA, 38 bp, Phlebovirus
gttaatacgactcactataggggggtccagcttgctac

SEQ ID NO: 637 - DNA, 18 bp, Phlebovirus
gtgagcatccaatactgc

SEQ ID NO: 638 - DNA, 38 bp, Phlebovirus
gttaatacgactcactataggggggagcacaatggacc

SEQ ID NO: 639 - DNA, 15 bp, Phlebovirus
gtggccagctgagag

SEQ ID NO: 640 - DNA, 37 bp, Phlebovirus
gttaatacgactcactatagggggcccagcatgctac

SEQ ID NO: 641 - DNA, 17 bp, Phlebovirus
gccaactgagtgcctta

SEQ ID NO: 642 - DNA, 37 bp, Phlebovirus
gttaatacgactcactatagggtctacgacaggccag

SEQ ID NO: 643 - DNA, 18 bp, Phlebovirus
tgtgatcaacccagcatt

SEQ ID NO: 644 - DNA, 41 bp, Phlebovirus
gttaatacgactcactataggggatttgatgctactgtggt

SEQ ID NO: 645 - DNA, 19 bp, Phlebovirus
ttctcctaccatctgcttg

SEQ ID NO: 646 - DNA, 38 bp, Phlebovirus
gttaatacgactcactatagggtttgatgcagccgtgg

SEQ ID NO: 647 - DNA, 18 bp, Phlebovirus
tgtcccggatcatctgat

SEQ ID NO: 648 - DNA, 40 bp, Phlebovirus
gttaatacgactcactatagggtgtgggcttttctgtcat

SEQ ID NO: 649 - DNA, 18 bp, Phlebovirus
tgtccctcatcatctggt

SEQ ID NO: 650 - DNA, 40 bp, Picornavirus
gttaatacgactcactatagggggtgacaggctaaggatg

SEQ ID NO: 651 - DNA, 18 bp, Picornavirus
ctccggtcacctattcag

SEQ ID NO: 652 - DNA, 40 bp, Picornavirus
gttaatacgactcactatagggattcaacaaggggctgaa

SEQ ID NO: 653 - DNA, 12 bp, Picornavirus
cggaccacgtcc

SEQ ID NO: 654 - DNA, 40 bp, Picornavirus
gttaatacgactcactatagggatcatgcctccccgatta

SEQ ID NO: 655 - DNA, 18 bp, Picornavirus
tcatattccaagcggctt

SEQ ID NO: 656 - DNA, 40 bp, Picornavirus
gttaatacgactcactatagggattcatgtcacctgcgag

SEQ ID NO: 657 - DNA, 17 bp, Picornavirus
gtgcccatcatgttatt

SEQ ID NO: 658 - DNA, 37 bp, Picornavirus
gttaatacgactcactatagggcatgtcacccgcgag

SEQ ID NO: 659 - DNA, 18 bp, Picornavirus
agtgcccatcatgttgtt

SEQ ID NO: 660 - DNA, 41 bp, Picornavirus
gttaatacgactcactatagggcattcatgtcacctgctag

SEQ ID NO: 661 - DNA, 18 bp, Picornavirus
atggcccatcatgttgtt

SEQ ID NO: 662 - DNA, 40 bp, Picornavirus
gttaatacgactcactatagggtttcatgtcaccagccag

SEQ ID NO: 663 - DNA, 18 bp, Picornavirus
acgtacccatcatgttgt

SEQ ID NO: 664 - DNA, 40 bp, Picornavirus
gttaatacgactcactatagggccttcatgtcaccagcta

SEQ ID NO: 665 - DNA, 18 bp, Picornavirus
aggtgcccatcatattgt

SEQ ID NO: 666 - DNA, 38 bp, Picornavirus
gttaatacgactcactatagggtcatgtcgccagcaac

SEQ ID NO: 667 - DNA, 40 bp, Picornavirus
gttaatacgactcactatagggtgcggctaatcctaactg

SEQ ID NO: 668 - DNA, 15 bp, Picornavirus
cacccgtagtcggtt

SEQ ID NO: 669 - DNA, 40 bp, Picornavirus
gttaatacgactcactataggggactactttgggtgtccg

SEQ ID NO: 670 - DNA, 18 bp, Picornavirus
gccaatccaattcgcttt

SEQ ID NO: 671 - DNA, 40 bp, Picornavirus
gttaatacgactcactatagggctcaaggtgtcccaacat

SEQ ID NO: 672 - DNA, 15 bp, Picornavirus
gagttgggttgcacg

SEQ ID NO: 673 - DNA, 40 bp, Picornavirus
gttaatacgactcactatagggctaatcccaacctccgag

SEQ ID NO: 674 - DNA, 15 bp, Picornavirus
gtagtctgttccgcc

SEQ ID NO: 675 - DNA, 40 bp, Picornavirus
gttaatacgactcactatagggcccctgaatgtggctaac

SEQ ID NO: 676 - DNA, 15 bp, Picornavirus
cggacacccgtagtt

SEQ ID NO: 677 - DNA, 39 bp, Picornavirus
gttaatacgactcactatagggctgaatgcggctaacct

SEQ ID NO: 678 - DNA, 15 bp, Picornavirus
cgtagtcggtcccat

SEQ ID NO: 679 - DNA, 39 bp, Picornavirus
gttaatacgactcactatagggccctgaatgcggctaat

SEQ ID NO: 680 - DNA, 15 bp, Picornavirus
cacccgtagtcggtt

SEQ ID NO: 681 - DNA, 37 bp, Picornavirus
gttaatacgactcactatagggagtctttggggacgc

SEQ ID NO: 682 - DNA, 18 bp, Picornavirus
cctaagaggtttcacccg

SEQ ID NO: 683 - DNA, 41 bp, Picornavirus
gttaatacgactcactatagggcacgatctatgaagtcacc

SEQ ID NO: 684 - DNA, 15 bp, Picornavirus
tctccatcacgcaac

SEQ ID NO: 685 - DNA, 37 bp, Picornavirus
gttaatacgactcactataggggccagccaaggttta

SEQ ID NO: 686 - DNA, 18 bp, Picornavirus
taccttctgggcatcctt

SEQ ID NO: 687 - DNA, 39 bp, Pneumovirus
gttaatacgactcactatagggagctgcaattagtgggg

SEQ ID NO: 688 - DNA, 20 bp, Pneumovirus
ttagggttgttcattgtcat

SEQ ID NO: 689 - DNA, 37 bp, Pneumovirus
gttaatacgactcactatagggagaggctgcagaaca

SEQ ID NO: 690 - DNA, 18 bp, Pneumovirus
ttgctgcttcattaccca

SEQ ID NO: 691 - DNA, 34 bp, Pneumovirus
gttaatacgactcactatagggtggggctatggc

SEQ ID NO: 692 - DNA, 19 bp, Pneumovirus
ggcacccatattgtaagtg

SEQ ID NO: 693 - DNA, 40 bp, Pneumovirus
gttaatacgactcactataggggaggtggctccagaatac

SEQ ID NO: 694 - DNA, 18 bp, Pneumovirus
tctatcccctgctgctaa

SEQ ID NO: 695 - DNA, 40 bp, Polyomavirus
gttaatacgactcactatagggtatttggtgcttgcctga

SEQ ID NO: 696 - DNA, 18 bp, Polyomavirus
gtcctgaccagcttctac

SEQ ID NO: 697 - DNA, 37 bp, Polyomavirus
gttaatacgactcactatagggcacaggaggggatgt

SEQ ID NO: 698 - DNA, 15 bp, Polyomavirus
ctttacgaggcccca

SEQ ID NO: 699 - DNA, 40 bp, Polyomavirus
gttaatacgactcactatagggacagaaggacccctagag

SEQ ID NO: 700 - DNA, 18 bp, Polyomavirus
ctcatcatgtctgggtcc

SEQ ID NO: 701 - DNA, 40 bp, Polyomavirus
gttaatacgactcactataggggtgtaacacccacagaca

SEQ ID NO: 702 - DNA, 18 bp, Polyomavirus
gcagtgttctagggtctc

SEQ ID NO: 703 - DNA, 40 bp, Polyomavirus
gttaatacgactcactatagggaattagcagccacaaggt

SEQ ID NO: 704 - DNA, 15 bp, Polyomavirus
taggtcacagctgca

SEQ ID NO: 705 - DNA, 40 bp, Polyomavirus
gttaatacgactcactatagggttggggtccaacactttt

SEQ ID NO: 706 - DNA, 18 bp, Polyomavirus
ggtgagcctaggaatgtc

SEQ ID NO: 707 - DNA, 40 bp, Poxvirus
gttaatacgactcactatagggctacgggcattgtcatct

SEQ ID NO: 708 - DNA, 18 bp, Poxvirus
gctcgctttacagatcct

SEQ ID NO: 709 - DNA, 40 bp, Poxvirus
gttaatacgactcactatagggcaccgcaatagatcctgt

SEQ ID NO: 710 - DNA, 18 bp, Poxvirus
aatatgtccgccgttcat

SEQ ID NO: 711 - DNA, 40 bp, Poxvirus
gttaatacgactcactatagggacacgctggacaatctag

SEQ ID NO: 712 - DNA, 18 bp, Poxvirus
ggtggaggtctgagaatg

SEQ ID NO: 713 - DNA, 40 bp, Poxvirus
gttaatacgactcactatagggggaccccaacatctttga

SEQ ID NO: 714 - DNA, 15 bp, Poxvirus
gacctcaccgacgat

SEQ ID NO: 715 - DNA, 40 bp, Poxvirus
gttaatacgactcactatagggggcaaccccgattatgta

SEQ ID NO: 716 - DNA, 18 bp, Poxvirus
gtcaaggactggatagcc

SEQ ID NO: 717 - DNA, 38 bp, Reovirus
gttaatacgactcactatagggtcggagacctcgaagc

SEQ ID NO: 718 - DNA, 18 bp, Reovirus
tgtgcgtgtcgtaatttg

SEQ ID NO: 719 - DNA, 39 bp, Reovirus
gttaatacgactcactatagggtaattggcgacctggag

SEQ ID NO: 720 - DNA, 18 bp, Reovirus
atgtgggtgtcgtagttc

SEQ ID NO: 721 - DNA, 40 bp, Reovirus
gttaatacgactcactatagggggaccgctgaatacctaa

SEQ ID NO: 722 - DNA, 18 bp, Reovirus
aacaattggatgacggct

SEQ ID NO: 723 - DNA, 40 bp, Reovirus
gttaatacgactcactatagggggactgccgaatacctaa

SEQ ID NO: 724 - DNA, 18 bp, Reovirus
cacgattggatgacgact

SEQ ID NO: 725 - DNA, 40 bp, Reovirus
gttaatacgactcactatagggtggaccatctgattctgc

SEQ ID NO: 726 - DNA, 18 bp, Reovirus
aatccatagacacgccag

SEQ ID NO: 727 - DNA, 40 bp, Reovirus
gttaatacgactcactatagggtatcgtgtccttgagcac

SEQ ID NO: 728 - DNA, 15 bp, Reovirus
gtcccctgtacacca

SEQ ID NO: 729 - DNA, 40 bp, Reovirus
gttaatacgactcactatagggcgcacgctgattatgttt

SEQ ID NO: 730 - DNA, 18 bp, Reovirus
tgtgcagccatttctttt

SEQ ID NO: 731 - DNA, 41 bp, Reovirus
gttaatacgactcactatagggcgcatgcggattatgtatc

SEQ ID NO: 732 - DNA, 18 bp, Reovirus
gtgctgccatttctttca

SEQ ID NO: 733 - DNA, 41 bp, Reovirus
gttaatacgactcactatagggcacatgctgattacgtttc

SEQ ID NO: 734 - DNA, 17 bp, Reovirus
gccgccatttctttcat

SEQ ID NO: 735 - DNA, 40 bp, Reovirus
gttaatacgactcactatagggatctacttgcaccaggtg

SEQ ID NO: 736 - DNA, 20 bp, Reovirus
ggtactttcatgtcaagtgc

SEQ ID NO: 737 - DNA, 40 bp, Reovirus
gttaatacgactcactatagggttgatttccagcaccagt

SEQ ID NO: 738 - DNA, 19 bp, Reovirus
actctggcttgaatgtttt

SEQ ID NO: 739 - DNA, 40 bp, Reovirus
gttaatacgactcactataggggctaatacgcctcccttt

SEQ ID NO: 740 - DNA, 18 bp, Reovirus
aaggcatcacgacctatg

SEQ ID NO: 741 - DNA, 40 bp, Reovirus
gttaatacgactcactatagggtagaccttactgacgcct

SEQ ID NO: 742 - DNA, 18 bp, Reovirus
ccggggccataattacat

SEQ ID NO: 743 - DNA, 39 bp, Reovirus
gttaatacgactcactataggggagaggctggcagattg

SEQ ID NO: 744 - DNA, 18 bp, Reovirus
agagtctagcagggaaca

SEQ ID NO: 745 - DNA, 40 bp, Rhabdovirus
gttaatacgactcactatagggcaggattagactgggctg

SEQ ID NO: 746 - DNA, 18 bp, Rhabdovirus
ggctatctgatgggcaat

SEQ ID NO: 747 - DNA, 40 bp, Rhabdovirus
gttaatacgactcactatagggcagacgatgaggagcttt

SEQ ID NO: 748 - DNA, 18 bp, Rhabdovirus
ctttcccccattgaccat

SEQ ID NO: 749 - DNA, 37 bp, Rhabdovirus
gttaatacgactcactatagggaacgagctgagtcca

SEQ ID NO: 750 - DNA, 15 bp, Rhabdovirus
tcatctgctgcctga

SEQ ID NO: 751 - DNA, 40 bp, Rhabdovirus
gttaatacgactcactatagggatttggcctagagggaac

SEQ ID NO: 752 - DNA, 18 bp, Rhabdovirus
ttgaagtaatcagccggg

SEQ ID NO: 753 - DNA, 40 bp, Human smacovirus
gttaatacgactcactatagggcttaacctgtcctccgac

SEQ ID NO: 754 - DNA, 18 bp, Human smacovirus
aatgggtacatgtgggac

SEQ ID NO: 755 - DNA, 39 bp, Human smacovirus
gttaatacgactcactatagggcctgaaccggtcttctg

SEQ ID NO: 756 - DNA, 18 bp, Human smacovirus
acggttacttatgggacg

SEQ ID NO: 757 - DNA, 40 bp, Eastern equine encephalitis virus
gttaatacgactcactataggggcagtggaccatttgaac

SEQ ID NO: 758 - DNA, 18 bp, Eastern equine encephalitis virus
taatgttctcggtggctc

SEQ ID NO: 759 - DNA, 40 bp, Togavirus
gttaatacgactcactatagggtacgcagttacccatcac

SEQ ID NO: 760 - DNA, 15 bp, Togavirus
gtacagaccggggag

SEQ ID NO: 761 - DNA, 40 bp, Togavirus
gttaatacgactcactatagggcctggacagcggattatt

SEQ ID NO: 762 - DNA, 18 bp, Togavirus
ggcgaattatcccagtga

SEQ ID NO: 763 - DNA, 40 bp, Togavirus
gttaatacgactcactatagggagaggtggcagtctatca

SEQ ID NO: 764 - DNA, 18 bp, Togavirus
gcgtactcctttcattgc

SEQ ID NO: 765 - DNA, 40 bp, Togavirus
gttaatacgactcactatagggtccgtgtctgtgtaggta

SEQ ID NO: 766 - DNA, 18 bp, Togavirus
gacgccttcaatcctgta

SEQ ID NO: 767 - DNA, 40 bp, Togavirus
gttaatacgactcactatagggggacgtgtatgctgtaca

SEQ ID NO: 768 - DNA, 18 bp, Togavirus
caatccaatacgccgttc

SEQ ID NO: 769 - DNA, 40 bp, Togavirus
gttaatacgactcactatagggatactgactaaccggggt

SEQ ID NO: 770 - DNA, 18 bp, Togavirus
tgcagaacggacttcttt

SEQ ID NO: 771 - DNA, 40 bp, Togavirus
gttaatacgactcactatagggttgaggtagaagccaagc

SEQ ID NO: 772 - DNA, 18 bp, Togavirus
cgcacttccaatgtcaag

SEQ ID NO: 773 - DNA, 37 bp, Togavirus
gttaatacgactcactataggggcgatcgagtgatgc

SEQ ID NO: 774 - DNA, 18 bp, Togavirus
ggtgaatggcctcgatta

SEQ ID NO: 775 - DNA, 40 bp, Togavirus
gttaatacgactcactataggggcaatttcgcggtatacc

SEQ ID NO: 776 - DNA, 18 bp, Togavirus
gtcgatgaggacgtgtag

SEQ ID NO: 777 - DNA, 41 bp, Orthohepevirus
gaaattaatacgactcactatagggaggcccaccagttcat

SEQ ID NO: 778 - DNA, 43 bp, Orthohepevirus
gaaattaatacgactcactatagggggaggcccatcagtttat

SEQ ID NO: 779 - DNA, 16 bp, Orthohepevirus
taccacagcattcgcc

SEQ ID NO: 780 - DNA, 16 bp, Orthohepevirus
acagcattcgccaagg

SEQ ID NO: 781 - DNA, 42 bp, Rhinovirus
gaaattaatacgactcactataggggacagggtgtgaagagc

SEQ ID NO: 782 - DNA, 43 bp, Rhinovirus
gaaattaatacgactcactatagggtgacaaggtgtgaagagc

SEQ ID NO: 783 - DNA, 18 bp, Rhinovirus
aagtagttggtcccatcc

SEQ ID NO: 784 - DNA, 18 bp, Rhinovirus
aagtagtcggtcccatcc

SEQ ID NO: 785 - DNA, 43 bp, Rhinovirus
gaaattaatacgactcactatagggtagtttggtcgatgaggc

SEQ ID NO: 786 - DNA, 18 bp, Rhinovirus
cggaggactcacagttaa

SEQ ID NO: 787 - DNA, 18 bp, Rhinovirus
ggaggactcacaaccaag

SEQ ID NO: 788 - DNA, 98 bp, Orthohepevirus
tggaggcccatcagtttattaaggctcctggcatcactactgccattgagcaggctgctctggcagcggccaactccgccttggcgaatgctgtggtg

SEQ ID NO: 789 - DNA, 141 bp, Rhinovirus
ggacaaggtgtgaagagccccgtgtgctcactttgagtcctccggcccctgaatgtggctaaccttaaccctgcagccagtgcacacaatccagtgtgtatctggtcgtaatgagcaattgcgggatgggaccaactactt

SEQ ID NO: 790 - DNA, 140 bp, Rhinovirus
ctagtttggtcgatgaggctaggaattccccacgggtgaccgtgtcctagcctgcgtggcggccaacccagcttatgctgggacgcctttttatagacatggtgtgaagacccgcatgtgcttggttgtgagtcctccgg

SEQ ID NO: 791 - RNA, 28 nt, Orthohepevirus
cggaguuggccgcugcuagagcugccug

SEQ ID NO: 792 - RNA, 28 nt, Rhinovirus
gguuagccacauucaggggccggaggac

SEQ ID NO: 793 - RNA, 28 nt, Rhinovirus
uuggccgccacgcaggcuaggacacggu

SEQ ID NO: 794 - RNA, 28 nt, Culex flavivirus
cagauugaacgccaacaucacguacauc

SEQ ID NO: 795 - RNA, 28 nt, Tula virus
auuuuuugacuugauaccaaaucugcaa

SEQ ID NO: 796 - RNA, 28 nt, Papillomavirus
agcucuaauugauuccaaagccuuuuaa

SEQ ID NO: 797 - RNA, 28 nt, Getah virus
gacuguaucagugaucuuacacaucagg

SEQ ID NO: 798 - RNA, 28 nt, Zika virus
ccuuccagccguggggcagcucguucac

SEQ ID NO: 799 - RNA, 28 nt, Cowpox virus
cgauuauaacaacagauauuauaauccu

SEQ ID NO: 800 - RNA, 28 nt, Kyasanur forest virus
auacccagccuuccacacgugucagaug

SEQ ID NO: 801 - RNA, 28 nt, Hepatitis C virus
acuccaccaacgaucugaccgccacccg

SEQ ID NO: 802 - DNA, 45 bp, Artificial Sequence
  Synthetic Oligonucleotide
gaaattaatacgactcactatagggtggacatacaatgcagaatt

SEQ ID NO: 803 - DNA, 45 bp, Artificial Sequence
  Synthetic Oligonucleotide
gaaattaatacgactcactatagggtggacatacaatgctgaact

SEQ ID NO: 804 - DNA, 45 bp, Artificial Sequence
  Synthetic Oligonucleotide
gaaattaatacgactcactatagggtggacttacaatgctgaact

SEQ ID NO: 805 - DNA, 45 bp, Artificial Sequence
  Synthetic Oligonucleotide
gaaattaatacgactcactatagggtggacttatcaggctgaact

SEQ ID NO: 806 - DNA, 45 bp, Artificial Sequence
  Synthetic Oligonucleotide
gaaattaatacgactcactatagggtgggcatataatgcagaatt

SEQ ID NO: 807 - DNA, 45 bp, Artificial Sequence
  Synthetic Oligonucleotide
gaaattaatacgactcactatagggtgggcctacaatgcagagct

SEQ ID NO: 808 - DNA, 45 bp, Artificial Sequence
  Synthetic Oligonucleotide
gaaattaatacgactcactatagggtgggcttacaacgcagaact

SEQ ID NO: 809 - DNA, 45 bp, Artificial Sequence
  Synthetic Oligonucleotide
gaaattaatacgactcactatagggtggtcatacaacgcacagct

SEQ ID NO: 810 - DNA, 45 bp, Artificial Sequence
  Synthetic Oligonucleotide
gaaattaatacgactcactatagggtggtcatacaacgcggagct

SEQ ID NO: 811 - DNA, 90 bp, Artificial Sequence
  Synthetic Oligonucleotide
gaaattaatacgactcactatagggtggtcatacaatgcaaaactgaaattaatacgactcactatagggtggtcatacaatgcaaaact

SEQ ID NO: 812 - DNA, 45 bp, Artificial Sequence
  Synthetic Oligonucleotide
gaaattaatacgactcactatagggtggtcatacaatgccgaatt

SEQ ID NO: 813 - DNA, 45 bp, Artificial Sequence
  Synthetic Oligonucleotide
gaaattaatacgactcactatagggtggtcatataatgcacaact

SEQ ID NO: 814 - DNA, 45 bp, Artificial Sequence
  Synthetic Oligonucleotide
gaaattaatacgactcactatagggtggtcatataatgcagagct

SEQ ID NO: 815 - DNA, 45 bp, Artificial Sequence
  Synthetic Oligonucleotide
gaaattaatacgactcactatagggtggtcttacaatgctgaatt

SEQ ID NO: 816 - DNA, 45 bp, Artificial Sequence
  Synthetic Oligonucleotide
gaaattaatacgactcactatagggtggacgtatcaagctgaatt

SEQ ID NO: 817 - DNA, 20 bp, Artificial Sequence
  Synthetic Oligonucleotide
aaagcagccgtttcctattt

SEQ ID NO: 818 - DNA, 20 bp, Artificial Sequence
  Synthetic Oligonucleotide
aaagcacccgttccctattt

SEQ ID NO: 819 - DNA, 20 bp, Artificial Sequence
  Synthetic Oligonucleotide
aaagcacccattccctattt

SEQ ID NO: 820 - DNA, 20 bp, Artificial Sequence
  Synthetic Oligonucleotide
aaagcagccatttccaattt

SEQ ID NO: 821 - DNA, 20 bp, Artificial Sequence
  Synthetic Oligonucleotide
aaagcacccatttcctagtt

SEQ ID NO: 822 - DNA, 20 bp, Artificial Sequence
  Synthetic Oligonucleotide
aaaacatccattccctagtt

SEQ ID NO: 823 - DNA, 20 bp, Artificial Sequence
  Synthetic Oligonucleotide
gaaacatcctttcccttctt

SEQ ID NO: 824 - DNA, 20 bp, Artificial Sequence
  Synthetic Oligonucleotide
gaaacatccattcccttctt

SEQ ID NO: 825 - DNA, 20 bp, Artificial Sequence
  Synthetic Oligonucleotide
aaagcatccagtgccatctt

SEQ ID NO: 826 - DNA, 20 bp, Artificial Sequence
  Synthetic Oligonucleotide
aaaacatcctttcccatctt

SEQ ID NO: 827 - DNA, 20 bp, Artificial Sequence
  Synthetic Oligonucleotide
aaagcaccctttcccatctt

SEQ ID NO: 828 - DNA, 20 bp, Artificial Sequence
  Synthetic Oligonucleotide
aaagcatccgttgcccaatt

SEQ ID NO: 829 - DNA, 20 bp, Artificial Sequence
  Synthetic Oligonucleotide
aaaacacccgtttcctttgt

SEQ ID NO: 830 - DNA, 20 bp, Artificial Sequence
  Synthetic Oligonucleotide
aaaacatccatttcctttgt

SEQ ID NO: 831 - DNA, 20 bp, Artificial Sequence
  Synthetic Oligonucleotide
aaagcacccatttcctttgt

SEQ ID NO: 832 - DNA, 20 bp, Artificial Sequence
  Synthetic Oligonucleotide
gaaacatccattccctttgt

SEQ ID NO: 833 - DNA, 20 bp, Artificial Sequence
  Synthetic Oligonucleotide
aaagcacccgttccctaggt

SEQ ID NO: 834 - DNA, 20 bp, Artificial Sequence
  Synthetic Oligonucleotide
gaagcaaccatttccttcgt

SEQ ID NO: 835 - DNA, 20 bp, Artificial Sequence
  Synthetic Oligonucleotide
gaaacaaccgttacccagct

SEQ ID NO: 836 - DNA, 20 bp, Artificial Sequence
  Synthetic Oligonucleotide
aaaacatccagtcccatcct

SEQ ID NO: 837 - DNA, 20 bp, Artificial Sequence
  Synthetic Oligonucleotide
aaagcaaccatctcctgtat

SEQ ID NO: 838 - DNA, 20 bp, Artificial Sequence
  Synthetic Oligonucleotide
gaagcagccattcccagtat

SEQ ID NO: 839 - DNA, 20 bp, Artificial Sequence
  Synthetic Oligonucleotide
gaaacaaccattgcccatat

SEQ ID NO: 840 - DNA, 20 bp, Artificial Sequence
  Synthetic Oligonucleotide
gaaacagccgttgccttgat

SEQ ID NO: 841 - DNA, 20 bp, Artificial Sequence
  Synthetic Oligonucleotide
aaagcatccgttcccttcat

SEQ ID NO: 842 - DNA, 20 bp, Artificial Sequence
  Synthetic Oligonucleotide
gaaacatccgttcccttcat

SEQ ID NO: 843 - DNA, 20 bp, Artificial Sequence
  Synthetic Oligonucleotide
aaaacaaccattcccttcat

SEQ ID NO: 844 - DNA, 20 bp, Artificial Sequence
  Synthetic Oligonucleotide
aaaacatccattcccctcat

SEQ ID NO: 845 - DNA, 20 bp, Artificial Sequence
  Synthetic Oligonucleotide
gaagcaaccgttcccagcat

SEQ ID NO: 846 - DNA, 20 bp, Artificial Sequence
  Synthetic Oligonucleotide
aaagcaaccattcccagcat

SEQ ID NO: 847 - DNA, 45 bp, Artificial Sequence
  Synthetic Oligonucleotide
gaaattaatacgactcactatagggatgaggaatgctcmtgttay

SEQ ID NO: 848 - DNA, 45 bp, Artificial Sequence
  Synthetic Oligonucleotide
gaaattaatacgactcactatagggthgargartgctcytgytat

SEQ ID NO: 849 - DNA, 45 bp, Artificial Sequence
  Synthetic Oligonucleotide
gaaattaatacgactcactatagggtrgargartgttchtgytay

SEQ ID NO: 850 - DNA, 45 bp, Artificial Sequence
  Synthetic Oligonucleotide
gaaattaatacgactcactatagggtygargartgttcctgttac

SEQ ID NO: 851 - DNA, 45 bp, Artificial Sequence
  Synthetic Oligonucleotide
gaaattaatacgactcactatagggtwgargartgytcytgytay

SEQ ID NO: 852 - DNA, 45 bp, Artificial Sequence
  Synthetic Oligonucleotide
gaaattaatacgactcactatagggthgaagartgytcrtgytay

SEQ ID NO: 853 - DNA, 45 bp, Artificial Sequence
  Synthetic Oligonucleotide
gaaattaatacgactcactatagggtwgaggartgctcmtgytay

SEQ ID NO: 854 - DNA, 45 bp, Artificial Sequence
  Synthetic Oligonucleotide
gaaattaatacgactcactatagggtwgargartgytcwtgytay

SEQ ID NO: 855 - DNA, 45 bp, Artificial Sequence
  Synthetic Oligonucleotide
gaaattaatacgactcactatagggttgaagaatgctcatgytay

SEQ ID NO: 856 - DNA, 20 bp, Artificial Sequence
  Synthetic Oligonucleotide
scatgccarttrtcyctgca

SEQ ID NO: 857 - DNA, 20 bp, Artificial Sequence
  Synthetic Oligonucleotide
ccyttccarttgtctctgca

SEQ ID NO: 858 - DNA, 20 bp, Artificial Sequence
  Synthetic Oligonucleotide
ccyttccarttgtcyctrca

SEQ ID NO: 859 - DNA, 20 bp, Artificial Sequence
  Synthetic Oligonucleotide
ccyckccarttgtcyckaca

SEQ ID NO: 860 - DNA, 20 bp, Artificial Sequence
  Synthetic Oligonucleotide
ccrttccaattrtcyckgca

SEQ ID NO: 861 - DNA, 20 bp, Artificial Sequence
  Synthetic Oligonucleotide
ccyttccaattgtcyctrca

SEQ ID NO: 862 - DNA, 20 bp, Artificial Sequence
  Synthetic Oligonucleotide
ccytgccarttrtcyctgca

SEQ ID NO: 863 - DNA, 20 bp, Artificial Sequence
  Synthetic Oligonucleotide
  misc_feature (3)..(3): n is a, c, g, or t
ccngtccarttgtcyctaca

SEQ ID NO: 864 - DNA, 20 bp, Artificial Sequence
  Synthetic Oligonucleotide
ccctgccaattrtcyctgca

SEQ ID NO: 865 - DNA, 141 bp, Influenza virus
tggacttacaatgccgaactgttggttctattggaaaatgaaagaactttggactaccacgattcaaatgtgaagaacttatatgaaaaggtaagaagccagttaaaaaacaatgccaaggaaattggaaacggctgcttt

SEQ ID NO: 866 - DNA, 141 bp, Influenza virus
tggacatacaatgccgaactcctagttctaatggaaaatgagaggacacttgatttccatgactctaatgtaaggaatctgtacgataaggtcagaatgcaactgagggacaatgctaaggaaatagggaacggatgcttt

SEQ ID NO: 867 - DNA, 141 bp, Influenza virus
tggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttc

SEQ ID NO: 868 - DNA, 141 bp, Influenza virus
tggtcttacaatgctgaattgctggtggcattagaaaatcaacatactatagatgtgacagactctgaaatgaacaaactctttgaaagagttaggcgccaactaagagagaatgctgaggacaaaggaaatggatgtttt

SEQ ID NO: 869 - DNA, 141 bp, Influenza virus
tggacttataatgctgaacttctggttctcatggaaaatgagagaactctagacttccatgactcaaatgtcaagaacctttacgacaaggtccgactacagcttagggataatgcaaaggagctgggtaacggttgtttc

SEQ ID NO: 870 - DNA, 141 bp, Influenza virus
tggacatacaatgctgaactgctggttcttcttgaaaacgaaagaacactagacctgcatgatgcgaatgtgaagaacctatatgaaaaggtcaaatcacaattaagggacaatgctaatgatctaggaaatgggtgcttt

SEQ ID NO: 871 - DNA, 141 bp, Influenza virus
tggtcatacaatgctgaactcttggtagcaatggagaaccagcatacaattgatctggctgattcagaaatgaacaaactgtacgaacgagtgaaaagacagctgagagagaatgctgaagaagatggcactggttgcttt

SEQ ID NO: 872 - DNA, 141 bp, Influenza virus
tgggcttacaatgcagaactccttgtacttctagaaaaccagaaaacactagacgaacatgactccaatgtcaagaacctctttgatgaagtgaaaaggaggttgtcaaccaatgcaatagatgctgggaacggttgcttc

SEQ ID NO: 873 - DNA, 141 bp, Influenza virus
tgggcatataatgcagaattgctagttctgcttgaaaaccagaaaacactcgatgagcatgacgcaaatgtaaacaatctatataataaagtgaagagggcgttgggttccaatgcggtggaagatgggaaaggatgtttc

SEQ ID NO: 874 - DNA, 141 bp, Influenza virus
tggacgtatcaagctgaattgctggtagcaatggaaaatcagcatacaattgacatggctgattcagaaatgctgaatctatatgagagggtgaggaagcaactaaggcaaaatgcagaagaagatgggaaagggtgcttt

SEQ ID NO: 875 - DNA, 141 bp, Influenza virus
tggtcatacaacgcacagcttcttgttctactggaaaatgaaaaaacattagatctccatgattctaatgttcgaaacctccatgaaaaggtcagacgaatgctgaaggacaatgctaaagatgaagggaatggttgtttt

SEQ ID NO: 876 - DNA, 141 bp, Influenza virus
tgggcatacaatgctgaactgcttgttctattggaaaatcagaagacattagatgagcatgatgctaatgtaaggaatctacatgatagagtcagaagagtcctaagggaaaatgcaattgatacaggagatggttgcttt

SEQ ID NO: 877 - DNA, 141 bp, Influenza virus
tggtcatacaatgcaaagcttcttgttttactagaaaacgacaagactctagacatgcacgacgctaatgtcaggaacctgcatgatcaagtccgcagagtgctgaggaccaatgcaattgatgaggggaatggatgtttt

SEQ ID NO: 878 - DNA, 141 bp, Influenza virus
tggtcatacaatgctgaactattggtggccctggaaaatcagcacacaatagatgttacagactccgagatgaacaaactctttgaaagggtgagaagacaacttagggaaaatgcggaagatcaaggcaacggctgtttc

SEQ ID NO: 879 - DNA, 141 bp, Influenza virus
tggtcatacaatgccgaattactggtggcaatggaaaatcaacacacaattgaccttgcagactctgagatgaacaaactctatgagagagtgaggaggcaattaagggagaatgccgaggaggatgggactggatgtttt

SEQ ID NO: 880 - DNA, 141 bp, Influenza virus
tggtcatacaatgctaaacttcttgtactgcttgaaaatggtagaacattagacttgcatgatgcaaatgtcagaaacttacatgatcaggtcaaaagggtgttgaaggacaatgcaattgacgaaggaaatggttgcttc

SEQ ID NO: 881 - DNA, 67 bp, Influenza virus
atgaggaatgctcctgttatcctgattctagtgaaatcacatgtgtgtgcagggataactggcatgg

SEQ ID NO: 882 - DNA, 67 bp, Influenza virus
tcgaggagtgctcttgctatcctcgatatcctggtgtcagatgtgtctgcagagacaactggaaagg

SEQ ID NO: 883 - DNA, 64 bp, Influenza virus
tagaagaatgttcctgctatgtggacattgatgtttactgtatatgtagggacaattggaaagg

SEQ ID NO: 884 - DNA, 67 bp, Influenza virus
tcgaagagtgttcctgttacccaagtggaacagatattgagtgtgtctgtcgggacaattggcgggg

SEQ ID NO: 885 - DNA, 67 bp, Influenza virus
ttgaagagtgctcttgctaccccaacttgggtaaagtggagtgtgtttgccgagataattggaatgg

SEQ ID NO: 886 - DNA, 67 bp, Influenza virus
tagaagaatgctcatgctatggagcagaagaggtgatcaaatgcatatgcagggacaattggaaagg

SEQ ID NO: 887 - DNA, 67 bp, Influenza virus
tagaggagtgctcatgctatgggcacaattcaaaggtgacttgtgtatgcagggacaactggcaagg

SEQ ID NO: 888 - DNA, 67 bp, Influenza virus
tagaagaatgctcatgctaccccaatgaaggtaaagtggaatgtgtttgtagggacaactggactgg

SEQ ID NO: 889 - DNA, 67 bp, Influenza virus
ttgaagaatgctcatgttacggggaacgaacaggaattacctgcacatgcagggacaattggcaggg

SEQ ID NO: 890 - DNA, 67 bp, Influenza virus
atgaggaatgctcctgttacccagacactggcatagtgatgtgtgtatgcagggacaactggcatgg

SEQ ID NO: 891 - DNA, 67 bp, Influenza virus; identical to SEQ ID NO: 881

SEQ ID NO: 892 - DNA, 67 bp, Influenza virus
atgaggaatgctcatgttatcctgatacaggcaaagtaatgtgtgtttgcagagacaattggcatgc

SEQ ID NO: 893 - DNA, 67 bp, Influenza virus
tcgaggagtgctcttgttatcctcgatatcctggtgtcagatgcgtctgcagagacaactggaaagg

SEQ ID NO: 894 - DNA, 67 bp, Influenza virus
tcgaagagtgctcttgctatcctcgatatcctggtgtcagatgtgtctgcagagacaactggaaagg

SEQ ID NO: 895 - DNA, 67 bp, Influenza virus
ttgaggartgctcctgttatcctagatatcctggtgtcagatgtgtatgcagrgacaactggaaagg

SEQ ID NO: 896 - DNA, 67 bp, Influenza virus
ttgaggagtgctcctgttatcctcgatttcctggtgtcagatgtgtctgcagagacaactggaaagg

SEQ ID NO: 897 - DNA, 67 bp, Influenza virus
tagaggagtgctcctgttatccccgatatcctggtgtcagatgcatctgtagagacaactggaaagg

SEQ ID NO: 898 - DNA, 64 bp, Influenza virus
tagaagaatgttcctgctatgtggacattgatgtttactgtatatgtagggacaattggaaggg

SEQ ID NO: 899 - DNA, 64 bp, Influenza virus
tagaggagtgttcttgctatgtggacaccgatgtgtactgcatatgtagggacaattggaaagg

SEQ ID NO: 900 - DNA, 64 bp, Influenza virus
tggaagagtgttcatgttacacagatgtagacatctactgtgtgtgcagagacaactggaaagg

SEQ ID NO: 901 - DNA, 64 bp, Influenza virus
tggaggagtgttcttgttatgtggacatcgatgtgtactgcatatgtagggacaattggaaagg

SEQ ID NO: 902 - DNA, 67 bp, Influenza virus
tcgaagagtgttcctgttacccaagtggaacggatattgagtgtgtctgtcgggacaattggcgggg

SEQ ID NO: 903 - DNA, 67 bp, Influenza virus
tcgaagagtgttcctgttacccgagtggaacagatattgagtgtgtctgtcgggacaattggcgggg

SEQ ID NO: 904 - DNA, 67 bp, Influenza virus
tcgaagagtgttcctgttacccaagtggaatagatattgagtgtgtctgtcgggacaattggcgggg

SEQ ID NO: 905 - DNA, 67 bp, Influenza virus
ttgaggagtgttcctgttacccaagtggagaaaatgtcgagtgtgtgtgtagagacaattggagagg

SEQ ID NO: 906 - DNA, 67 bp, Influenza virus
ttgaagagtgctcttgctaccccaacttgggtaaagtggagtgcgtttgccgagataattggaatgg

SEQ ID NO: 907 - DNA, 67 bp, Influenza virus
tagaggagtgttcctgttaccccaacatgggaaaagtggaatgtgtttgcagggacaattggaatgg

SEQ ID NO: 908 - DNA, 67 bp, Influenza virus
tagaggagtgttcctgttatcccaacatggggaaagtggaatgtgtttgcagggacaattggaacgg

SEQ ID NO: 909 - DNA, 67 bp, Influenza virus
ttgaagaatgctcatgctatggagcaaaaggagtgatcaaatgcatctgcagagacaattggaaggg

SEQ ID NO: 910 - DNA, 67 bp, Influenza virus
tagaagagtgctcatgctatggagcagaagaaatgattaaatgcatttgcagggataattggaaggg

SEQ ID NO: 911 - DNA, 67 bp, Influenza virus
tagaagaatgctcgtgctatggagcagaagaggtgattaaatgcatttgcagggacaattggaaagg

SEQ ID NO: 912 - DNA, 67 bp, Influenza virus
tcgaagaatgttcatgctatggggcagcaggggtaatcaaatgtatatgcagggacaattggaaagg

SEQ ID NO: 913 - DNA, 67 bp, Influenza virus
tcgaagagtgttcatgctacggagcagcagggatgatcaaatgtgtatgcagagacaattggaaggg

SEQ ID NO: 914 - DNA, 67 bp, Influenza virus
ttgaggaatgctcctgttacgggcacagtcaaaaggtgacctgtgtgtgcagagataactggcaggg

SEQ ID NO: 915 - DNA, 67 bp, Influenza virus
tagaggagtgctcatgctatgggcacaattcgaaggtgacttgtgtatgcagggacaactggcaagg

SEQ ID NO: 916 - DNA, 67 bp, Influenza virus
tagaggagtgctcatgctatgggcacgattcaaaagtgacttgtgtatgcagggacaactggcaagg

SEQ ID NO: 917 - DNA, 67 bp, Influenza virus
tagaggaatgctcatgctatgggcacaattcaaaggtgacttgtgtatgcagggacaactggcaagg

SEQ ID NO: 918 - DNA, 67 bp, Influenza virus
tagaagaatgctcatgctaccccaatgaaggtaaagtggaatgtgtttgtagggacaattggactgg

SEQ ID NO: 919 - DNA, 67 bp, Influenza virus
tagaagaatgctcatgctaccccaatgaaggtaaagtggagtgtgtttgtagggacaactggactgg

SEQ ID NO: 920 - DNA, 67 bp, Influenza virus
ttgaggaatgttcttgttatccaaatgatggtaaagtggaatgcgtgtgtagagacaactggacggg

SEQ ID NO: 921 - DNA, 67 bp, Influenza virus
ttgaagaatgctcatgctatggggtgcaggcaggtattacttgcacgtgcagggataattggcaggg

SEQ ID NO: 922 - DNA, 67 bp, Influenza virus
ttgaagaatgctcatgctacggggaacaagcaggtattacttgcacgtgcagggataattggcaggg

SEQ ID NO: 923 - DNA, 67 bp, Influenza virus; identical to SEQ ID NO: 889

SEQ ID NO: 924 - DNA, 67 bp, Influenza virus
ttgaagaatgctcatgttacggggaacgaacagggattacctgcacatgcagggacaattggcaggg

SEQ ID NO: 925 - RNA, 28 nt, Influenza virus
cauuguuuuuuaguuggcuucuuacuuu

SEQ ID NO: 926 - RNA, 28 nt, Influenza virus
cauuagagucauggaaaucaaguguccu

SEQ ID NO: 927 - RNA, 28 nt, Influenza virus
uguauguugguucuccagggcaacaaga

SEQ ID NO: 928 - RNA, 28 nt, Influenza virus
uaguauguugauuuucuaaugccaccag

SEQ ID NO: 929 - RNA, 28 nt, Influenza virus
cagcucuuuugcauuauccuuaagcugu

SEQ ID NO: 930 - RNA, 28 nt, Influenza virus
ggucauuagcauugucccuuaguuguga

SEQ ID NO: 931 - RNA, 28 nt, Influenza virus
ucuccaucgcuaucaagaguucagcguu

SEQ ID NO: 932 - RNA, 28 nt, Influenza virus
ucacuucaucaaagagguucuugacauu

SEQ ID NO: 933 - RNA, 28 nt, Influenza virus
uugcgucaugcucaucgaguguuuucug

SEQ ID NO: 934 - RNA, 28 nt, Influenza virus
gauucagcauuucugaaucagccauguc

SEQ ID NO: 935 - RNA, 28 nt, Influenza virus
cauucgucugaccuuuucauggagguuu

SEQ ID NO: 936 - RNA, 28 nt, Influenza virus
uaaugucuucugauuuuccaauagaaca

SEQ ID NO: 937 - RNA, 28 nt, Influenza virus
ugcaugucuagagucuugucguucucua

SEQ ID NO: 938 - RNA, 28 nt, Influenza virus
gaucuuccgcauuuucccuaaguugucu

SEQ ID NO: 939 - RNA, 28 nt, Influenza virus
agagucugcaaggucaauuguguguuga

SEQ ID NO: 940 - RNA, 28 nt, Influenza virus
ucgugcaagucuaauguucuaccauuuu

SEQ ID NO: 941 - RNA, 28 nt, Influenza virus
ucacuaugccagugucuggguaacagga

SEQ ID NO: 942 - RNA, 28 nt, Influenza virus
ugauuucacuagaaucaggauaacagga

SEQ ID NO: 943 - RNA, 28 nt, Influenza virus
acauuacuuugccuguaucaggauaaca

SEQ ID NO: 944 - RNA, 28 nt, Influenza virus
caucugacaccaggauaucgaggauaac

SEQ ID NO: 945 - RNA, 28 nt, Influenza virus
cacaucugacaccaggauaucgaggaua

SEQ ID NO: 946 - RNA, 28 nt, Influenza virus
uacacaucugacaccaggauacuuagga

SEQ ID NO: 947 - RNA, 28 nt, Influenza virus
gacacaucugacaccaggagaucgagga

SEQ ID NO: 948 - RNA, 28 nt, Influenza virus
gcaucugacaccaggauaucggggauaa

SEQ ID NO: 949 - RNA, 28 nt, Influenza virus
auacaguaaacaucaauguccacauagc

SEQ ID NO: 950 - RNA, 28 nt, Influenza virus
ccuacauaugcaguacacaucggugucc

SEQ ID NO: 951 - RNA, 28 nt, Influenza virus
acacaguagaugucuacaucuguguaac

SEQ ID NO: 952 - RNA, 28 nt, Influenza virus
auacaauacacaucaauguccacauaac

SEQ ID NO: 953 - RNA, 28 nt, Influenza virus
gacagacacacucaauauccguuccacu

SEQ ID NO: 954 - RNA, 28 nt, Influenza virus
cagacacacucaauaucuguuccacuug

SEQ ID NO: 955 - RNA, 28 nt, Influenza virus
acacucaauauuuauuccacuuggguaa

SEQ ID NO: 956 - RNA, 28 nt, Influenza virus
acacucgacauuuucuccacuuggguaa

SEQ ID NO: 957 - RNA, 28 nt, Influenza virus
ggcaaacgcacuccacuuuacccaaguu

SEQ ID NO: 958 - RNA, 28 nt, Influenza virus
cacauuccacuuuucccauguuggggua

SEQ ID NO: 959 - RNA, 28 nt, Influenza virus
acauuccacuuuccccauguugggauaa

SEQ ID NO: 960 - RNA, 28 nt, Influenza virus
cauuugaucacuccuuuugcuccauagc

SEQ ID NO: 961 - RNA, 28 nt, Influenza virus
cauuuaaucauuucuucugcuccauagc

SEQ ID NO: 962 - RNA, 28 nt, Influenza virus
cauuuaaucaccucuucugcuccauagc

SEQ ID NO: 963 - RNA, 28 nt, Influenza virus
cauuugauuaccccugcugccccauagc

SEQ ID NO: 964 - RNA, 28 nt, Influenza virus
auacacauuugaucaucccugcugcucc

SEQ ID NO: 965 - RNA, 28 nt, Influenza virus
acacaggucaccuuuugacugugcccgu

SEQ ID NO: 966 - RNA, 28 nt, Influenza virus
caagucaccuucgaauugugcccauagc

SEQ ID NO: 967 - RNA, 28 nt, Influenza virus
caagucacuuuugaaucgugcccauagc

SEQ ID NO: 968 - RNA, 28 nt, Influenza virus
cauacacaagucaccuuugaauugugcc

SEQ ID NO: 969 - RNA, 28 nt, Influenza virus
acaaacacauuccacuuuaccuucauug

SEQ ID NO: 970 - RNA, 28 nt, Influenza virus
acaaacacacuccacuuuaccuucauug

SEQ ID NO: 971 - RNA, 28 nt, Influenza virus
acacgcauuccacuuuaccaucauuugg

SEQ ID NO: 972 - RNA, 28 nt, Influenza virus
gugcaaguaauaccugccugcaccccau

SEQ ID NO: 973 - RNA, 28 nt, Influenza virus
acgugcaaguaauaccugcuuguucccc

SEQ ID NO: 974 - RNA, 28 nt, Influenza virus
ugcagguaauuccuguucguuccccgua

SEQ ID NO: 975 - RNA, 28 nt, Influenza virus
gcagguaaucccuguucguuucccguaa

SEQ ID NO: 976 - DNA, 46 bp, Human immunodeficiency virus
gaaattaatacgactcactatagggaattaaagccaggaatggatg

SEQ ID NO: 977 - DNA, 25 bp, Human immunodeficiency virus
agtcttgagttctcttattaagttc

SEQ ID NO: 978 - DNA, 45 bp, Human immunodeficiency virus
gaaattaatacgactcactatagggagagaactcaagacttctgg

SEQ ID NO: 979 - DNA, 24 bp, Human immunodeficiency virus
tggtaaatgcagtatacttcctga

SEQ ID NO: 980 - DNA, 48 bp, Human immunodeficiency virus
gaaattaatacgactcactatagggtcccttagataaagacttcagga

SEQ ID NO: 981 - DNA, 24 bp, Human immunodeficiency virus
tgtcatgctactttggaatattgc

SEQ ID NO: 982 - DNA, 49 bp, Human immunodeficiency virus
gaaattaatacgactcactatagggtccaaagtagcatgacaaaaatct

SEQ ID NO: 983 - DNA, 22 bp, Human immunodeficiency virus
acagatgttgtctcagttcctc

SEQ ID NO: 984 - DNA, 46 bp, Human immunodeficiency virus
gaaattaatacgactcactatagggagaaatagtagccagctgtga

SEQ ID NO: 985 - DNA, 20 bp, Human immunodeficiency virus
cactggctacatgaactgct

SEQ ID NO: 986 - DNA, 45 bp, Human immunodeficiency virus
gaaattaatacgactcactatagggcagttcatgtagccagtgga

SEQ ID NO: 987 - DNA, 20 bp, Human immunodeficiency virus
aattcctgcttgatccctgc

SEQ ID NO: 988 - DNA, 45 bp, Human immunodeficiency virus
gaaattaatacgactcactatagggccagtactacggttaaggcc

SEQ ID NO: 989 - DNA, 22 bp, Human immunodeficiency virus
gctgtcttaagatgttcagcct

SEQ ID NO: 990 - DNA, 49 bp, Human immunodeficiency virus
gaaattaatacgactcactatagggagcaacagacatacaaactaaaga

SEQ ID NO: 991 - DNA, 24 bp, Human immunodeficiency virus
tccataatccctaatgatctttgc

SEQ ID NO: 992 - RNA, 28 nt, Human immunodeficiency virus
uuuuuguuuauggcaaauacuggaguau

SEQ ID NO: 993 - RNA, 28 nt, Human immunodeficiency virus
uuucuguuuauggcaaauacuggaguau

SEQ ID NO: 994 - RNA, 28 nt, Human immunodeficiency virus
uuuuuguuuuuuaacccugcgggaugug

SEQ ID NO: 995 - RNA, 28 nt, Human immunodeficiency virus
uuguuguuuuuuaacccugcgggaugug

SEQ ID NO: 996 - RNA, 28 nt, Human immunodeficiency virus
guuacagauuuuuucuuuuuuaacccug

SEQ ID NO: 997 - RNA, 28 nt, Human immunodeficiency virus
gucauagauuuuuucuuuuuuaacccug

SEQ ID NO: 998 - RNA, 28 nt, Human immunodeficiency virus
gauacauaacuaugucuggauuuuguuu

SEQ ID NO: 999 - RNA, 28 nt, Human immunodeficiency virus
gacacauaacuaugucuggauuuuguuu

SEQ ID NO: 1000 - RNA, 28 nt, Human immunodeficiency virus
augcauguauugauagauaacuaugucu

SEQ ID NO: 1001 - RNA, 28 nt, Human immunodeficiency virus
augcacguauugauagauaacuaugucu

SEQ ID NO: 1002 - RNA, 28 nt, Human immunodeficiency virus
gauccaacauacaaaucauccauguauu

SEQ ID NO: 1003 - RNA, 28 nt, Human immunodeficiency virus
gaugcaacauacaaaucauccauguauu

SEQ ID NO: 1004 - RNA, 28 nt, Human immunodeficiency virus
aucuguacaaucuaguugccauauuccu

SEQ ID NO: 1005 - RNA, 28 nt, Human immunodeficiency virus
aucugcacaaucuaguugccauauuccu

SEQ ID NO: 1006 - RNA, 28 nt, Human immunodeficiency virus
aucuguacaaucuaguugccauauuccu

SEQ ID NO: 1007 - RNA, 28 nt, Human immunodeficiency virus
aucuauacaaucuaguugccauauuccu

SEQ ID NO: 1008 - RNA, 28 nt, Human immunodeficiency virus
aucuguacaaucuaguugccauauuccu

SEQ ID NO: 1009 - RNA, 28 nt, Human immunodeficiency virus
aucuuuacaaucuaguugccauauuccu

SEQ ID NO: 1010 - RNA, 28 nt, Human immunodeficiency virus
accagcauaauuuuuccuucuaaaugug

SEQ ID NO: 1011 - RNA, 28 nt, Human immunodeficiency virus
accaucauaauuuuuccuucuaaaugug

SEQ ID NO: 1012 - RNA, 28 nt, Human immunodeficiency virus
ucucagcuggaauaacuucugcuucuau

SEQ ID NO: 1013 - RNA, 28 nt, Human immunodeficiency virus
ucccagcuggaauaacuucugcuucuau

SEQ ID NO: 1014 - RNA, 28 nt, Human immunodeficiency virus
ugucucugcuggaauaacuucugcuucu

SEQ ID NO: 1015 - RNA, 28 nt, Human immunodeficiency virus
ugucugugcuggaauaacuucugcuucu

SEQ ID NO: 1016 - RNA, 28 nt, Human immunodeficiency virus
ugguguuuccugcccugucucugcugga

SEQ ID NO: 1017 - RNA, 28 nt, Human immunodeficiency virus
uggugcuuccugcccugucucugcugga

SEQ ID NO: 1018 - RNA, 28 nt, Human immunodeficiency virus
ugaauuugcugccauugucuguauguau

SEQ ID NO: 1019 - RNA, 28 nt, Human immunodeficiency virus
uguauuugcugccauugucuguauguau

SEQ ID NO: 1020 - RNA, 28 nt, Human immunodeficiency virus
uguauuugcugccauugucuguauguau

SEQ ID NO: 1021 - RNA, 28 nt, Human immunodeficiency virus
augcgugcuugaucccugcccaccaaca

SEQ ID NO: 1022 - RNA, 28 nt, Human immunodeficiency virus
aauucgugcuugaucccugcccaccaac

SEQ ID NO: 1023 - RNA, 28 nt, Human immunodeficiency virus
aauuugugcuugaucccugcccaccaac

SEQ ID NO: 1024 - RNA, 28 nt, Human immunodeficiency virus
ugccuaauuccugcuugaucccugccca

SEQ ID NO: 1025 - RNA, 28 nt, Human immunodeficiency virus
uggcuaauuccugcuugaucccugccca

SEQ ID NO: 1026 - RNA, 28 nt, Human immunodeficiency virus
aaagccaaauuccugcuugaucccugcc

SEQ ID NO: 1027 - RNA, 28 nt, Human immunodeficiency virus
aaagcuaaauuccugcuugaucccugcc

SEQ ID NO: 1028 - RNA, 28 nt, Human immunodeficiency virus
uguacggaaugccaaauuccugcuugau

SEQ ID NO: 1029 - RNA, 28 nt, Human immunodeficiency virus
ugcacggaaugccaaauuccugcuugau

SEQ ID NO: 1030 - RNA, 28 nt, Human immunodeficiency virus
uuguacggaaugccaaauuccugcuuga

SEQ ID NO: 1031 - RNA, 28 nt, Human immunodeficiency virus
uugugcggaaugccaaauuccugcuuga

SEQ ID NO: 1032 - RNA, 28 nt, Human immunodeficiency virus
uguagggaaugccaaauuccugcuugau

SEQ ID NO: 1033 - RNA, 28 nt, Human immunodeficiency virus
ugcggggaaugccaaauuccugcuugau

SEQ ID NO: 1034 - RNA, 28 nt, Human immunodeficiency virus
ugacuauggggauuguagggaaugccaa

SEQ ID NO: 1035 - RNA, 28 nt, Human immunodeficiency virus
ugaccauggggauuguagggaaugccaa

SEQ ID NO: 1036 - RNA, 28 nt, Human immunodeficiency virus
ccuugucuuuggggauuguagggaaugc

SEQ ID NO: 1037 - RNA, 28 nt, Human immunodeficiency virus
ccgugucuuuggggauuguagggaaugc

SEQ ID NO: 1038 - RNA, 28 nt, Human immunodeficiency virus
ccuugucuuuggggauuguagggaaugc

SEQ ID NO: 1039 - RNA, 28 nt, Human immunodeficiency virus
ccuuuucuuuggggauuguagggaaugc

SEQ ID NO: 1040 - RNA, 28 nt, Human immunodeficiency virus
ccuugucuuuggggauuguagggaaugc

SEQ ID NO: 1041 - RNA, 28 nt, Human immunodeficiency virus
ccucgucuuuggggauuguagggaaugc

SEQ ID NO: 1042 - RNA, 28 nt, Human immunodeficiency virus
uuauugauagauucuacuacuccuugac

SEQ ID NO: 1043 - RNA, 28 nt, Human immunodeficiency virus
uuauggauagauucuacuacuccuugac

SEQ ID NO: 1044 - RNA, 25 nt, Human immunodeficiency virus
uuucuacuuggcacuacuuuuaugu

SEQ ID NO: 1045 - RNA, 25 nt, Human immunodeficiency virus
uuuuuacuuggcacuacuuuuaugu

SEQ ID NO: 1046 - DNA, 725 bp, Human immunodeficiency virus
gaaattaatacgactcactatagggcccattagtcctattgaaactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagataaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaac

SEQ ID NO: 1047 - DNA, 725 bp, Human immunodeficiency virus
gaaattaatacgactcactatagggcccattagtcctattgaaactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgaaaatccatacaatactccagtatttgccataaagagaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagataaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaac

SEQ ID NO: 1048 - DNA, 725 bp, Human immunodeficiency virus
gaaattaatacgactcactatagggcccattagtcctattgaaactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaacaaatcagtaacagtactggatgtgggtgatgcatatttttcagttcccttagataaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaac

SEQ ID NO: 1049 - DNA, 725 bp, Human immunodeficiency virus
gaaattaatacgactcactatagggcccattagtcctattgaaactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcaatgacagtactggatgtgggtgatgcatatttttcagttcccttagataaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaac

SEQ ID NO: 1050 - DNA, 725 bp, Human immunodeficiency virus
gaaattaatacgactcactatagggcccattagtcctattgaaactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagataaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctgtcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaac

SEQ ID NO: 1051 - DNA, 725 bp, Human immunodeficiency virus
gaaattaatacgactcactatagggcccattagtcctattgaaactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagataaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacgtggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaac

SEQ ID NO: 1052 - DNA, 725 bp, Human immunodeficiency virus
gaaattaatacgactcactatagggcccattagtcctattgaaactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagataaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtagcatctgacttagaaatagggcagcatagaacaaaaatagaggaactgagacaacatctgttgaggtggggatttaccacaccagacaaaaaacatcagaaagaacctccattcctttggatgggttatgaac

SEQ ID NO: 1053 - DNA, 750 bp, Human immunodeficiency virus
gaaattaatacgactcactatagggagcaaaagaaatagtagccagctgtgataaatgtcagctaaaaggagaagccatgcatggacaagtagactgtagtccaggaatatggcaactagattgtacacatttagaaggaaaaattatcctggtagcagttcatgtagccagtggatatatagaagcagaagttattccagcagagacagggcaggaaacagcatactttctcttaaaattagcaggaagatggccagtaaaaacaatacatacagacaatggcagcaatttcaccagtactacggttaaggccgcctgttggtgggcagggatcaagcaggaatttggcattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaac

SEQ ID NO: 1054 - DNA, 750 bp, Human immunodeficiency virus
gaaattaatacgactcactatagggagcaaaagaaatagtagccagctgtgataaatgtcagctaaaaggagaagccatgcatggacaagtagactgtagtccaggaatatggcaactagattgtgcacatttagaaggaaaaattatcctggtagcagttcatgtagccagtggatatatagaagcagaagttattccagcagggacagggcaggaaacagcatactttctcttaaaattagcaggaagatggccagtaaaaacaatacatacagacaatggcagcaatttcaccagtactacggttaaggccgcctgttggtgggcagggatcaagcagaaatttggcattccctacaatccccaaagtaaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaac

SEQ ID NO: 1055 - DNA, 750 bp, Human immunodeficiency virus
gaaattaatacgactcactatagggagcaaaagaaatagtagccagctgtgataaatgtcagctaaaaggagaagccatgcatggacaagtagactgtagtccaggaatatggcaactagattgtatacatttagaaggaaaaattatcctggtagcagttcatgtagccagtggatatatagaagcagaagttattccagcacagacagggcaggaaacagcatactttctcttaaaattagcaggaagatggccagtaaaaacaatacatacagacaatggcagcaattacaccagtactacggttaaggccgcctgttggtgggcagggatcaagcaggcatttggcattccctacaatccccaaagtcacggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaaaaaaagcaaagatcattagggattatggaaaac

SEQ ID NO: 1056 - DNA, 750 bp, Human immunodeficiency virus
gaaattaatacgactcactatagggagcaaaagaaatagtagccagctgtgataaatgtcagctaaaaggagaagccatgcatggacaagtagactgtagtccaggaatatggcaactagattgtaaacatttagaaggaaaaattatcctggtagcagttcatgtagccagtggatatatagaagcagaagttattccagcagagacagggcaggaagcagcatactttctcttaaaattagcaggaagatggccagtaaaaacaatacatacagacaatggcagcaatttcaccagtactacggttaaggccgcctgttggtgggcagggatcaagcaggaatttgccattccctacaatccccaaagtcaaggagtagtagaatctatgcataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaac

SEQ ID NO: 1057 - DNA, 750 bp, Human immunodeficiency virus
gaaattaatacgactcactatagggagcaaaagaaatagtagccagctgtgataaatgtcagctaaaaggagaagccatgcatggacaagtagactgtagtccaggaatatggcaactagattgtacacatttagaaggaaaaattatcatggtagcagttcatgtagccagtggatatatagaagcagaagttattccagcagagacagggcaggaaacagcatactttctcttaaaattagcaggaagatggccagtaaaaacaatacatacagacaatggcagcaatttcaccagtactacggttaaggccgcctgttggtgggcagggatcaagcaggaatttagcattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaac

SEQ ID NO: 1058 - DNA, 750 bp, Human immunodeficiency virus
gaaattaatacgactcactatagggagcaaaagaaatagtagccagctgtgataaatgtcagctaaaaggagaagccatgcatggacaagtagactgtagtccaggaatatggcaactagattgtacacatttagaaggaaaaattatcctggtagcagttcatgtagccagtggatatatagaagcagaagttattccagcagagacagggcaggaaacagcatactttctcttaaaattagcaggaagatggccagtaaaaacaatacatacagacaatggcagcaatttcaccagtactacggttaaggccgcctgttggtgggcagggatcaagcaggaatttggcattccctgcaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaac

SEQ ID NO: 1059 - DNA, 750 bp, Human immunodeficiency virus
gaaattaatacgactcactatagggagcaaaagaaatagtagccagctgtgataaatgtcagctaaaaggagaagccatgcatggacaagtagactgtagtccaggaatatggcaactagattgtacacatttagaaggaaaaattatcctggtagcagttcatgtagccagtggatatatagaagcagaagttattccagcagagacagggcaggaaacagcatactttctcttaaaattagcaggaagatggccagtaaaaacaatacatacagacaatggcagcaatttcaccagtactacggttaaggccgcctgttggtgggcagggatcaagcaggaatttggcattccccacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaac

SEQ ID NO: 1060 - DNA, 750 bp, Human immunodeficiency virus
gaaattaatacgactcactatagggagcaaaagaaatagtagccagctgtgataaatgtcagctaaaaggagaagccatgcatggacaagtagactgtagtccaggaatatggcaactagattgtacacatttagaaggaaaaattatcctggtagcagttcatgtagccagtggatatatagaagcagaagttattccagcagagacagggcaggaaacagcatactttctcttaaaattagcaggaagatggccagtaaaaacaatacatacagacaatggcagcaatttcaccagtactacggttaaggccgcctgttggtgggcagggatcaagcaggaatttggcattccccgcaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaac

SEQ ID NO: 1061 - DNA, 750 bp, Human immunodeficiency virus
gaaattaatacgactcactatagggagcaaaagaaatagtagccagctgtgataaatgtcagctaaaaggagaagccatgcatggacaagtagactgtagtccaggaatatggcaactagattgtacacatttagaaggaaaaattatcctggtagcagttcatgtagccagtggatatatagaagcagaagttattccagcagagacagggcaggaaacagcatactttctcttaaaattagcaggaagatggccagtaaaaacaatacatacagacaatggcagcaatttcaccagtactacggttaaggccgcctgttggtgggcagggatcaagcaggaatttggcattccctacaatccccaaggtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaac

SEQ ID NO: 1062 - DNA, 750 bp, Human immunodeficiency virus
gaaattaatacgactcactatagggagcaaaagaaatagtagccagctgtgataaatgtcagctaaaaggagaagccatgcatggacaagtagactgtagtccaggaatatggcaactagattgtacacatttagaaggaaaaattatcctggtagcagttcatgtagccagtggatatatagaagcagaagttattccagcagagacagggcaggaaacagcatactttctcttaaaattagcaggaagatggccagtaaaaacaatacatacagacaatggcagcaatttcaccagtactacggttaaggccgcctgttggtgggcagggatcaagcaggaatttggcattccctacaatccccaaagtcacggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaac

SEQ ID NO: 1063 - DNA, 750 bp, Human immunodeficiency virus
gaaattaatacgactcactatagggagcaaaagaaatagtagccagctgtgataaatgtcagctaaaaggagaagccatgcatggacaagtagactgtagtccaggaatatggcaactagattgtacacatttagaaggaaaaattatcctggtagcagttcatgtagccagtggatatatagaagcagaagttattccagcagagacagggcaggaaacagcatactttctcttaaaattagcaggaagatggccagtaaaaacaatacatacagacaatggcagcaatttcaccagtactacggttaaggccgcctgttggtgggcagggatcaagcaggaatttggcattccctacaatccccaaagtcgaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtgccaagaagaaaagcaaagatcattagggattatggaaaac

SEQ ID NO: 1064 - DNA, 40 bp, Sudan ebolavirus
gttaatacgactcactatagggagtcaatcccccatttgg

SEQ ID NO: 1065 - DNA, 18 bp, Torque teno virus
gttttgctgtacggatcg

SEQ ID NO: 1066 - DNA, 40 bp, Mammarenavirus
gttaatacgactcactatagggacgtttggtggagtgatt

SEQ ID NO: 1067 - DNA, 18 bp, Mammarenavirus
ttacgtgtccactttgct

SEQ ID NO: 1068 - DNA, 40 bp, Mammarenavirus
gttaatacgactcactatagggtgaacaggacaagtcacc

SEQ ID NO: 1069 - DNA, 18 bp, Mammarenavirus
ctcagaagctgtgggtag

SEQ ID NO: 1070 - DNA, 40 bp, Mammarenavirus
gttaatacgactcactatagggatctgatgagatgtggcc

SEQ ID NO: 1071 - DNA, 18 bp, Mammarenavirus
ggtgagattgtgccttct

SEQ ID NO: 1072 - DNA, 40 bp, Mammarenavirus
gttaatacgactcactataggggacaccattagccacaca

SEQ ID NO: 1073 - DNA, 18 bp, Mammarenavirus
tcatgggtgaagagacac

SEQ ID NO: 1074 - DNA, 41 bp, Mammarenavirus
gttaatacgactcactatagggcaacaccattagctacaca

(19) United States
(12) Patent Application Publication
(10) Pub. No.: US 2022/0073987 A1
(43) Pub. Date: Mar. 10, 2022

(54) CRISPR SYSTEM BASED DROPLET DIAGNOSTIC SYSTEMS AND METHODS

(71) Applicants: THE BROAD INSTITUTE, INC., Cambridge, MA (US); PRESIDENT AND FELLOWS OF HARVARD COLLEGE, Cambridge, MA (US); MASSACHUSETTS INSTITUTE OF TECHNOLOGY, Cambridge, MA (US); THE GENERAL HOSPITAL CORPORATION, Boston, MA (US)

(72) Inventors: Catherine Amanda FREIJE, Cambridge, MA (US); Cameron MYHRVOLD, Cambridge, MA (US); Hayden METSKY, Cambridge, MA (US); Pardis SABETI, Cambridge, MA (US); Gowtham THAKKU, Cambridge, MA (US); Jared KEHE, Cambridge, MA (US); Cheri ACKERMAN, Cambridge, MA (US); Paul BLAINEY, Cambridge, MA (US); Deborah HUNG, Cambridge, MA (US)

(21) Appl. No.: 17/294,179

(22) PCT Filed: Nov. 14, 2019
(86) PCT No.: PCT/US2019/061577

§ 371 Date: May 14, 2021

Related U.S. Application Data
(60) Provisional application No. 62/767,070, filed on Nov. 14, 2018.
(60) Provisional application No. 62/841,812, filed on May 1, 2019.
(60) Provisional application No. 62/871,056, filed on Jul. 5, 2019.

(51) Int. Cl. C12Q 1/6883; C12Q 1/6804; B01L 3/00; C12Q 1/70; G01N 21/64; G01N 15/10

(52) U.S. Cl. CPC C12Q 1/6883; C12Q 1/6804; B01L 3/502761; C12Q 1/701; G01N 2021/6439; G01N 15/1056; C12Q 2600/16; C12Q 2600/156; B01L 2200/0652; G01N 21/6428

(57) ABSTRACT

RNA targeting proteins are utilized to provide a robust massively multiplexed CRISPR-based diagnostic by detection in droplets with attomolar sensitivity. Detection of both DNA and RNA with comparable levels of sensitivity at nanoliter volumes can differentiate targets from non-targets based on single base pair differences, with applications in multiple scenarios in human health including, for example, viral detection, bacterial strain typing, and sensitive genotyping.

### CROSS-REFERENCE TO RELATED APPLICATIONS

This application is a National Stage application of International Application No. PCT/US2019/061577, filed Nov. 14, 2019, which claims the benefit of U.S. Provisional Application No. 62/767,070, filed Nov. 14, 2018, U.S. Provisional Application No. 62/841,812, filed May 1, 2019, and U.S. Provisional Application No. 62/871,056, filed Jul. 5, 2019. The entire contents of the above-identified applications are hereby fully incorporated herein by reference.

### REFERENCE TO AN ELECTRONIC SEQUENCE LISTING

The content of the Electronic Sequence Listing (BROD_3830WP_ST25.txt); Size is 217 KB and was created on Oct. 7, 2019) is incorporated herein by reference in its entirety.

### TECHNICAL FIELD

The subject matter disclosed herein is generally directed to droplet diagnostics related to the use of CRISPR systems.

### BACKGROUND

The ability to rapidly detect nucleic acids with high sensitivity and single-base specificity for a large number of samples in a rapid timeframe has the potential to revolutionize diagnosis and monitoring for many diseases, provide valuable epidemiological information, and serve as a generalizable scientific tool. With a platform capable of testing a large number of samples at one time utilizing a small amount of sample would provide distinct advantage over the current state of the art. For example, qPCR approaches are sensitive but are expensive and rely on complex instrumentation, limiting usability to highly trained operators in laboratory settings. Other approaches, such as new methods combining isothermal nucleic acid amplification with portable platforms (Du et al., 2017; Pardee et al., 2016), offer high detection specificity in a point-of-care (POC) setting, but have somewhat limited applications due to low sensitivity. As nucleic acid diagnostics become increasingly relevant for a variety of healthcare applications, detection technologies that enables massive multiplexing with a high specificity and sensitivity at low cost would be of great utility in both clinical and basic research settings, ultimately allowing for pan-viral, pan-bacterial, or pan-pathogen testing of samples.

### SUMMARY

In certain example embodiments, a multiplex detection system is provided, which comprises a detection CRISPR system; optical barcodes for one or more target molecules, and a microfluidic device. In some embodiments, the detection CRISPR system comprises a DNA or RNA targeting protein, one or more guide RNAs designed to bind to corresponding target molecules, a masking construct, and an optical barcode. In some embodiments, the microfluidic device comprises an array of microwells and at least one flow channel beneath the microwells, with the microwells sized to capture at least two droplets.

The masking construct, which is optionally nucleic acid based, in some embodiments suppresses generation of a detectable positive signal. In other embodiments, the RNA-based masking construct suppresses generation of a detectable positive signal by masking the detectable positive signal, or generating a detectable negative signal instead. In one aspect, the masking construct is RNA-based. In certain embodiments, the RNA-based masking construct comprises a silencing RNA that suppresses generation of a gene product encoded by a reporting construct, wherein the gene product generates the detectable positive signal when expressed.

The RNA-based masking construct can be, in one embodiment, a ribozyme that generates the negative detectable signal, and wherein the positive detectable signal is generated when the ribozyme is deactivated, which can convert a substrate to a first color and wherein the substrate converts to a second color when the ribozyme is deactivated.

In some embodiments, the RNA-based masking construct comprises an RNA oligonucleotide to which a detectable ligand and a masking component are attached. In some embodiments, the detectable ligand is a fluorophore and the masking component is a quencher molecule.

The RNA-based masking construct can comprise a nanoparticle held in aggregate by bridge molecules, wherein at least a portion of the bridge molecules comprises RNA, and wherein the solution undergoes a color shift when the nanoparticle is disbursed in solution, optionally the nanoparticle is a colloidal metal, in some instances, colloidal gold. The RNA-based masking construct can also comprise a quantum dot linked to one or more quencher molecules by a linking molecule, wherein at least a portion of the linking molecule comprises RNA.

In some instances, the RNA-based masking construct comprises RNA in complex with an intercalating agent, wherein the intercalating agent changes absorbance upon cleavage of the RNA. In some instances, the intercalating agent is pyronine-Y or methylene blue.

The RNA-based masking agent can also be an RNA aptamer and/or comprises an RNA-tethered inhibitor, in some instances, the aptamer or RNA-tethered inhibitor sequesters an enzyme, wherein the enzyme generates a detectable signal upon release from the aptamer or RNA tethered inhibitor by acting upon a substrate. In particular embodiments, the aptamer is an inhibitory aptamer that inhibits an enzyme and prevents the enzyme from catalyzing generation of a detectable signal from a substrate or wherein the RNA-tethered inhibitor inhibits an enzyme and prevents the enzyme from catalyzing generation of a detectable signal from a substrate. The enzyme is, in some instances, thrombin, protein C, neutrophil elastase, subtilisin, horseradish peroxidase, beta-galactosidase, or calf alkaline phosphatase. When the enzyme is thrombin, the substrate can be para-nitroanilide covalently linked to a peptide substrate for thrombin, or 7-amino-4-methylcoumarin covalently linked to a peptide substrate for thrombin. The aptamer can sequester a pair of agents that when released from the aptamers combine to generate a detectable signal.

In an aspect, the embodiments disclosed herein are directed to methods for detecting target nucleic acids in a sample. The methods disclosed herein can, in some embodiments, comprise the steps of generating a first set of droplets, each droplet in the first set of droplets comprising at least one target molecule and an optical barcode; generating a second set of droplets, each droplet in the second set of droplets comprising a detection CRISPR system comprising a Cas protein, for example, an RNA targeting protein, and one or more guide RNAs designed to bind to corresponding target molecules, an RNA-based masking construct and optionally an optical barcode; combining the first set and second set of droplets into a pool of droplets and flowing the combined pool of droplets onto a microfluidic device comprising an array of microwells and at least one flow channel beneath the microwells, the microwells sized to capture at least two droplets; capturing droplets in the microwell and detecting the optical barcodes of the droplets captured in each microwell; merging the droplets captured in each microwell to formed merged droplets in each microwell, at least a subset of the merged droplets comprising a detection CRISPR system and a target sequence; initiating the detection reaction. The merged droplets are then maintained under conditions sufficient to allow binding of the one or more guide RNAs to one or more target molecules. Binding of the one or more guide RNAs to a target nucleic acid in turn activates the CRISPR protein. Once activated, the CRISPR protein then deactivates the masking construct, for example, by cleaving the masking construct such that a detectable positive signal is unmasked, released, or generated. Detection and measuring a detectable signal of each merged droplet at one or more time periods can be performed, indicating the presence of target molecules when, for example the positive detectable signal is present. The methods disclosed can include a step of amplifying the target molecules, amplification can be, in some instances RPA or PCR.

Target molecules are, in some embodiments, contained in a biological sample or an environmental sample. In some embodiments, the sample is from a human. The biological sample is, in some embodiments, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, or fluid obtained from a joint, or a swab of skin or mucosal membrane surface. The biological sample may be further processed prior to further evaluation, including, for example by enriching or isolating cells of interest.

The one or more guide RNAs are designed to bind to corresponding target molecules comprise a (synthetic) mismatch, which can be a mismatch up- or downstream of a Single Nucleotide Polymorphism (SNP) or other single nucleotide variation in the target molecule. The one or more guide RNAs can be designed to detect a single nucleotide polymorphism in a target RNA or DNA, or a splice variant of an RNA transcript. Guide RNAs can in some instances, be designed to detect drug resistance SNPs in a viral infection. In some embodiments, guide RNAs can also be designed to bind to one or more target molecules that are diagnostic for a disease state, which can optionally be characterized by the presence or absence of drug resistance or susceptibility gene or transcript or polypeptide, and can optionally be an infection. In some instances, the infection is caused by a virus, a bacterium a fungus, a protozoa, or a parasite. The guide RNAs are designed to distinguish between one or more microbial strains. The guide RNAs can in some instances comprise at least 90 guide RNAs.

The targeting protein can, in some embodiments comprise one or more RuvC-like domains. In particular embodiments, the CRISPR protein is Cas12, in embodiments, the Cas12 is Cpf1 or C2c1. The targeting protein can, in some embodiments, comprise one or more HEPN domains, which can optionally comprise a RxxxxH motif sequence. In some instances, the RxxxH motif comprises a R{N/H/K]X₁X₂X₃H (SEQ ID NO:1) sequence, which in some embodiments X₁ is R, S, D, E, Q, N, G, or Y, and X₂ is independently I, S, T, V, or L, and X₃ is independently L, F, N, Y, V, I, S, D, E, or A. In some particular embodiments, the CRISPR RNA-targeting protein is Cas 13. In particular embodiments, the Cas13 is Cas13a, Cas13b1, Cas13b2, or Cas13c.

In some instances, making optical assessments comprises capturing an image of each microwell. The optical barcode is detected in some embodiments by using light microscopy, fluorescence microscopy, Raman spectroscopy, or a combination thereof. The optical barcode comprises a particle of a particular size, shape, refractive index, color, or combination thereof in some embodiments. The optical barcode comprising a particle can comprise colloidal metal particles, nanoshells, nanotubes, nanorods, quantum dots, hydrogel particles, liposomes, dendrimers, or metal-liposome particles. each optical barcode comprises one or more fluorescent dyes, which can be a distinct ratio of fluorescent dyes. The detectable signal that can be measured is in some instances a level of fluorescence.

Devices for use in the methods of systems disclosed herein can comprise an array of at least 40,0000 microwells or at least 190,000 microwells. A multiplex detection system is also disclosed, which in one embodiment, includes a detection CRISPR system comprising an RNA targeting protein and one or more guide RNAs designed to bind to corresponding target molecules, an RNA-based masking construct and an optical barcode; optical barcodes for one or more target molecules; and a microfluidic device comprising an array of microwells and at least one flow channel between the microwells, the microwells sized to capture at least two droplets. Kits including the multiplex detection systems are also provided in embodiments of the presently disclosed subject matter. The kits can include instructions for the performing diagnostics, reagents, equipment microfluidic platform, reagents, etc. and standards for calibrating or conducting the methods. The instructions provided in a kit according to the invention may be directed to suitable operational parameters in the form of a label or a separate insert. Optionally, the kit may further comprise a standard or control information so that the test sample can be compared with the control information standard to determine if whether a consistent result is achieved.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 provides a schematic of an exemplary method of droplet detection. Pathogen detection with SHERLOCK can be massively multiplexed by performing detection in droplets on a chip bearing an array of microwells. Amplification reactions (using RPA or PCR) can be performed in standard tubes or microwells. Detection and amplification mixes are then arrayed in microwells. A unique fluorescent barcode composed of ratios of fluorescent dyes can be added to each detection mix and each target. Barcoded reagents are emulsified in oil, and droplets from the emulsions are pooled together in one tube. The droplet pool is loaded onto a PDMS chip bearing a microwell array. Each microwell accommodates two droplets, randomly creating pairwise combinations of all pooled droplets. The microwells are clamped shut against glass, isolating the contents of each well, and fluorescence microscopy is used to read the barcodes of all the droplets and determine the contents of each microwell. After imaging, the droplets are merged in an electric field, combining detection mixes and targets and beginning the detection reaction. The chip is incubated to allow the reaction to proceed, and fluorescence microscopy is used to monitor progression of the SHERLOCK (Specific High-sensitivity Enzymatic Reporter unLOCKing) reaction.

FIG. 2 includes images showing detection reagents and targets can be stably emulsified as droplets in oil. At left: white light image of aqueous solutions of targets emulsified in oil. At right: a fluorescence image of a microwell chip loaded with a library of detection reagents and targets, each bearing unique fluorescent barcodes. The contents of each well can be determined from the fluorescent barcodes.

FIG. 3 includes charts showing SHERLOCK performs equally well in plates and droplets. At left: Sensitivity curve of a SHERLOCK for Zika virus in plates. At right: Sensitivity curve of the same SHERLOCK assay for Zika virus in droplets. Error bars on the left indicate one standard deviation; error bars on the right are S.E.M.

FIG. 4 provides charts showing SHERLOCK discriminates single nucleotide polymorphisms (SNPs) equally well in plates and droplets. At left: SHERLOCK discrimination of a SNP that arose when Zika virus spread to the United States. At right: droplet SHERLOCK detection of the same SNP. Error bars on the left indicate one standard deviation; error bars on the right are S.E.M.

FIG. 5 includes a heat map showing Influenza subtypes can be discriminated by SHERLOCK detection in droplets in a microwell array. Fold turn-on after background subtraction of crRNA pools are indicated in the heat map.

FIG. 6 includes heat map results of multiplexed detection of Influenza H subtypes. 41 crRNAs were designed to target the H segment of Influenza based on sequences deposited since 2008. Boxes indicate sets of crRNAs designed against each subtype, and asterisks indicate crRNAs that align to the majority consensus sequence for each subtype with 0 or 1 mismatches. Control crRNA pools against H4, H8, and H12 are indicated.

FIG. 7 shows a heat map of a second design of multiplexed detection of Influenza H subtypes. 28 crRNAs were designed to target the H segment of Influenza based on sequences deposited since 2008, with preferential weighting for more recent sequences. Boxes indicate sets of crRNAs designed against each subtype, and asterisks indicate crRNAs that align to the majority consensus sequence for each subtype with 0 or 1 mismatches. Control crRNA pools against H4, H8, and H12 are indicated.

FIG. 8 includes a heat map of multiplexed detection of Influenza N subtypes. 35 crRNAs were designed to target the H segment of Influenza based on sequences deposited since 2008, with preferential weighting for more recent sequences. Boxes indicate sets of crRNAs designed against each subtype, and asterisks indicate crRNAs that align to the majority consensus sequence for each subtype with 0 or 1 mismatches. “crRNA36” indicates a negative control where no crRNA was added.

FIG. 9 includes multiplexed detection of 6 mutations in HIV reverse transcriptase using droplet SHERLOCK. Fluorescence at varying time points is shown for the indicated mutations for crRNAs targeting the ancestral and derived alleles using synthetic targets for both the ancestral and derived sequences. Synthetic targets (10⁴ cp/μl) were amplified using multiplexed PCR and detected using droplet SHERLOCK. Error bars: S.E.M.

FIG. 10 charts how HIV derived v0 and Ancestral v1 tests work and can potentially be used together.

FIG. 11 includes results of multiplexed detection of drug resistance mutations in TB using droplet SHERLOCK. Background-subtracted fluorescence is shown after 30 minutes for both alleles (reference, and drug-resistant).

FIG. 12 graphs demonstrating that combining SHERLOCK and microwell array chip technologies provides the highest throughput for multiplexed detection to date.

FIG. 13 shows how expansion of the number of barcodes and size of the chip enables massive multiplexing. (Left) Using 3 fluorescent dyes, the current set of 64 barcodes has been expanded to 105 barcodes. The possibility of adding a fourth dye has been demonstrated on a small scale with no loss in coding accuracy compared to the existing system and can readily be extended to scale to hundreds of barcodes; (Right) The existing chip can be quadrupled in size, reducing the number of chips necessary to assay development by four times.

FIG. 14 includes a graph showing that with the implementation of additional barcodes and expanded chip dimensions, the ability to test ˜20 samples at once for all human associated viruses is within reach, as indicated.

FIG. 15A-15D Combinatorial Arrayed Reactions for Multiplexed Evaluation of Nucleic acids (CARMEN). FIG. 15A Identification of multiple circulating pathogens in human and animal populations represents a large-scale detection problem. FIG. 15B Schematic of CARMEN workflow. FIG. 15C Zika virus is detected by a single CARMEN-Cas13 assay with attomolar sensitivity and tens of replicate droplet pairs (black dots); red lines mark medians in the graph and are used to construct the heatmap below. Representative droplet images are shown above the graph. FIG. 15D Zika virus detection charted in fluorescence versus input concentration.

FIG. 16A-16C Comprehensive identification of human-associated viruses with CARMEN-Cas13. FIG. 16A The development and testing of a panel for all human-associated viruses with ≥10 available genome sequences. FIG. 16B Experimental design and FIG. 16C testing of a comprehensive human-associated viral panel using CARMEN-Cas13. Heatmap indicates background-subtracted fluorescence after 1 h of detection. PCR primer pools and viral families are below and to the left of the heatmap, respectively. Gray lines: crRNAs that were not tested.

FIG. 17A-17D Influenza subtype discrimination with CARMEN-Cas13. FIG. 17A Schematic of Influenza A subtype discrimination using CARMEN-Cas13. FIG. 17B Discrimination of H1-H16 using CARMEN-Cas13. FIG. 17C Discrimination of N1-N9 using CARMEN-Cas13. FIG. 17D Identification of H and N subtypes from viral seedstocks and synthetic targets. Heatmaps indicate background-subtracted fluorescence after 1 h (in FIG. 17B) or 3 h (in FIG. 17C & FIG. 17D) of Cas13 detection. In FIG. 17B-FIG. 17D, synthetic targets were used at 104 cp/ul.

FIG. 18A-18F Multiplexed DRM identification with CARMEN-Cas13. FIG. 18A Schematic of HIV drug resistance mutation (DRM) identification using CARMEN-Cas13. FIG. 18B Identification of 6 reverse transcriptase mutations using CARMEN-Cas13. FIG. 18C DRM identification in patient plasma samples using CARMEN-Cas13. FIG. 18D Identification of 21 integrase DRMs using CARMEN-Cas13. Heatmaps indicate SNP indexes after 0.5-3 h of Cas13 detection; FIG. 18B and FIG. 18D are normalized by row. In FIG. 18B-FIG. 18D, synthetic targets were used at 104 cp/ul. Asterisks in FIG. 18D indicate the target with the mutation; boxes indicate multiple mutations in the same codon. FIG. 18E charts DRM frequency versus SNP index for K103N reverse transcriptase mutation. FIG. 18F DRM identification in patient plasma and serum samples using CARMEN-Cas13.

FIG. 19A-19E Comprehensive identification of human-associated viruses with CARMEN-Cas13. FIG. 19A Schematic of the development of a detection panel for human-associated viruses with ≥10 available genome sequences, with one potential application to regional viral diagnosis and surveillance. FIG. 19B Color code classification accuracy improves with mild data filtering. FIG. 19C Workflow for designing primers and crRNAs using CATCH dx. FIG. 19D Experimental design FIG. 19E. testing of a comprehensive human-associated viral panel using CARMEN-Cas13. Heatmap indicates background-subtracted fluorescence after 3 h of Cas13 detection.

FIG. 20A-20C CARMEN Schematic FIG. 20A includes a detailed molecular schematic of nucleic acid detection in CARMEN-Cas13. After amplification (with optional reverse transcription), detection is performed with Cas13, using in vitro transcription to convert amplified DNA into RNA. The resulting RNA is detected with exquisite sequence specificity by Cas13-crRNA complexes, and collateral cleavage produces a signal using a cleavage reporter RNA; FIG. 20B provides a detailed CARMEN Schematic. (Step 1) Samples are amplified, color coded, and emulsified. In parallel, detection mixes are assembled, color coded and emulsified. (Step 2) Droplets from each emulsion are pooled into a single tube and mixed by pipetting. (Step 3) The droplets are loaded into the chip in a single pipetting step. SIDE VIEW: The droplets are deposited through the loading slot into the flow space between the chip and glass. Tilting the loader moves the pool of droplets around the flow space, allowing the droplets to float up into the microwells. (Step 4) The chip is clamped against glass, isolating the contents of each microwell, and imaged by fluorescence microscopy to identify the color code and position of each droplet. (Step 5) Droplets are merged, initiating the detection reaction. (Step 6) The detection reactions in each microwell are monitored over time (a few minutes-3 hours) by fluorescence microscopy; FIG. 20C detailed side view of the acrylic loading apparatus, droplet flow, entry into microwell, and merger of two droplets.

FIG. 21A-21K Chip design, fabrication, loading and imaging. FIG. 21A Microwell design optimized for droplets made from PCR products or detection mixes. FIG. 21B Dimensions and layout of a standard chip. Light blue is the area covered by the microwell array. FIG. 21C Photograph of a standard chip. FIG. 21D Photograph of a standard chip sealed inside an acrylic loader, ready for imaging. FIG. 21E Dimensions and layout of mChip, compared to a standard chip. Light purple is the area covered by the microwell array. FIG. 21F AutoCAD rendering of acrylic molds used for mChip fabrication. FIG. 21G Photograph of an mChip. FIG. 21H (left) AutoCAD rendering of each part of the mChip loader; (middle) AutoCAD rendering of the set-up of an mChip loader; (right) AutoCAD rendering of an mChip in a loader, ready to be loaded. FIG. 21I Photograph of an mChip being loaded. FIG. 21J Loading and sealing mChip, corresponding to steps in FIG. 20B: (Step 3) mChip loading: Droplets are deposited at the edge of the chip into the flow space between the chip and the acrylic loader. Tilting the loader moves the pool of droplets around the flow space, allowing the droplets to float up into the microwells. (Step 4) The chip and loader lid are removed from the base and sealed against PCR film. No glass is used to seal the mChip. The sealed mChip, suspended from the acrylic loader lid, can be placed directly onto the microscope for imaging. FIG. 21K Photograph of an mChip sealed and ready to be imaged.

FIG. 22A-22E Multiplexed detection of Zika sequences using CARMEN—A closer look at Zika experiments. FIG. 22A Plate reader data for SHERLOCK detection of synthetic Zika sequences at 3 h. FIG. 22B Comparison of plate reader (FIG. 20A) and droplets (FIG. 15C) data. FIG. 22C Bootstrap analysis of Zika detection in droplets; FIG. 22D Receiver operating characteristics (ROC) curve for ZIke detection in droplets. AUC: area under the curve; FIG. 22E Assay, test, and droplet pair replicate nomenclature. Each multiplexed assay consists of a matrix of tests, where the dimensions of the matrix are M samples×N detection mixes. Each test is the result of one sample being evaluated by one detection mix, where the result of the test is the median value of a set of replicate droplet pairs in the microwell array.

FIG. 23A-23C Quantitative CARMEN-Cas13. FIG. 23A Schematic showing amplification primers containing T7 or T3 promoters, leading to increased signal for the majority (T7) product after Cas13 detection. Quantitative CARMEN-Cas13 schematic showing amplification primers containing T7 or T3 promoters, leading to increased signal for the majority (T7) product after Cas13 detection. FIG. 23B Increased dynamic range of detection using quantitative CARMEN-Cas13. Dynamic range is indicated using colored bars above the graph. Error bars indicate SEM. FIG. 23C chart shows linear correlation between real concentration and calculated concentration.

FIG. 24A-24F Design and Characterization of 1050 Color Codes. FIG. 24A Design of 1050 color codes. FIG. 24B Characterization of 210 color codes and the 3-color dimension of 1050 color codes. FIG. 24C Performance of 210 color codes in 3-color space. FIG. 24D Performance of 1050 color codes in 3-color space. FIG. 24E Characterization of 1050 color codes in 4th color dimension. FIG. 24F depicts expansion of fluorescent barcodes in 3-color space and four-color space, including performance in 4^{th} color dimension

FIG. 25A-25G mChip design and fabrication FIG. 25A Dimensions and layout of mChip, compared to a standard chip. Light purple shows the area covered by the microwell array. FIG. 25B AutoCAD rendering of acrylic molds used for mChip fabrication. FIG. 25C (left) AutoCAD rendering of each part of the mChip loader; (middle) AutoCAD rendering of the set-up of an mChip loader; (right) AutoCAD rendering of an mChip in a loader, ready to be loaded. FIG. 25D Photograph of an mChip. FIG. 25E Photograph of an mChip loader with an mChip inside, ready to be loaded (corresponds to the right-hand cartoon in C). FIG. 25F Photograph of an mChip being loaded. FIG. 25G Photograph of an mChip sealed and ready to be imaged (the output of the scheme illustrated in D).

FIG. 26 Detailed schematic of primer and crRNA design for the human-associated virus panel. There are 576 human-associated viral species with at least 1 genome neighbor in NCBI, and 169 with 10 or more genome neighbors. Genomes were aligned for each segment, and analyzed the sequence diversity using CATCH-dx to determine optimal primer and crRNA binding sites (see Methods for details).

FIG. 27A-27D Human associated virus panel design statistics. FIG. 27A Number of species in each family in the human-associated virus panel design. FIG. 27B Number of primer pairs required to capture at least 90% of the sequence diversity within each species. Two species required the use of primer pairs containing degenerate bases. FIG. 27C Number of crRNAs required to capture at least 90% of the sequence diversity within each species. FIG. 27D The fraction of sequences within each species covered by each designed crRNA set; small crRNA sets were able to be designed with 90% or greater coverage for 164 of the 169 species.

FIG. 28A-28C Human-associated virus panel version 1 performance. FIG. 28A Background-subtracted fluorescence heatmap from the testing version 1 of the human-associated viral panel. FIG. 28B crRNAs were classified into on-target, low activity, or cross-reactive by sequence analysis (black) or based on experimental data (orange). FIG. 28C Potential causes of low activity or cross-reactivity.

FIG. 29A-29B Human-associated virus panel: comparison of rounds 1 and 2. FIG. 29A Round 1. FIG. 29B Round 2 comparison.

FIG. 30A-30B Comparison of round 1 and round 2 of human-associated virus panel testing. FIG. 30A Distributions of the number of replicate droplet pairs for each crRNA-Target in round 1 (top) and round 2 (bottom) of testing. FIG. 30A Summary of crRNA performance in rounds 1 and 2.

FIG. 31A-31D Performance of individual guides in the human-associated virus panel, rounds 1 and 2. FIG. 31A Individual guide performance for rounds 1 and 2 (x-axis). FIG. 31B Areas under the receiver operating characteristic (ROC) curve for on-target vs off-target reactivity in round 1 of testing. For each range of performance (>0.97, 0.89-0.97, and <0.89), representative on-target and off-target distributions are shown. FIG. 31C Areas under the receiver operating characteristic (ROC) curve for on-target vs off-target reactivity in round 2 of testing. For each range of performance (>0.97, 0.89-0.97, and <0.89), representative on-target and off-target distributions are shown. FIG. 31D Comparison of AUCs from rounds 1 and 2. Guides with particularly low performance in round 2 are labeled.

FIG. 32A-32B Influenza A design overview and statistics. FIG. 32A The design goals for the Influenza A subtyping assay. FIG. 32B Overview of the four rounds of the design process.

FIG. 33A-33B Influenza A individual crRNA performance. FIG. 33A Distributions of droplet fluorescence for each Influenza A H-subtype crRNA with each target. A receiver operating characteristic (ROC) curve for on-target reactivity (e.g. crRNA H1 with Target H1) vs all other off-target activity (e.g. crRNA H1 with any other target) is shown at the right. FIG. 33B Distributions of droplet fluorescence for each Influenza A N-subtype crRNA with each target. A receiver operating characteristic (ROC) curve for on-target reactivity vs all other off-target activity is shown at the right. AUC=area under the curve.

FIG. 34 Influenza A N sub-subtype identification. Heatmap showing the full set of crRNAs designed to capture the sequence diversity within the Influenza A genome segment containing neuraminidase. 35 synthetic targets were tested (at 10⁴ cp/μl) using the 35 crRNAs designed. Each subtype is indicated with an orange box, the consensus sequence for each subtype is indicated using an asterisk.

FIG. 35 HIV droplet fluorescence distributions for reverse transcriptase mutations. Distributions of the droplet fluorescence for each crRNA-Target pair after 30 min in most cases; a 3 hour time point is shown for V106M and M184V. SNP indices displayed in FIG. 18B are calculated from the medians of these distributions.

FIG. 36 HIV low allele frequency for reverse transcriptase mutations. Bar graphs showing serial 1:3 dilutions of synthetic targets containing wild-type reverse transcriptase sequences or those with the indicated 6 drug-resistance mutations. In 5 of 6 cases, an allele frequency <30% was detected, and in 2 cases down to 3%.

FIG. 37 Testing of a comprehensive human-associated viral panel using CARMEN-Cas13. Heatmap indicates background-subtracted fluorescence after 1 h of detection. PCR primer pools and viral families are below and to the left of the heatmap, respectively. Gray lines: crRNAs not tested in round 2. “Dengue” indicates samples from 4 patients infected with dengue virus, 274 “Zika” indicates samples from 4 patients infected with Zika virus, and “Healthy” indicates plasma, serum, and urine samples pooled from healthy human donors. Virus names are listed in black if they were detected only in infected patients, or in grey if they were detected in any of the negative controls. Purple lines with exes indicate viruses detected in negative controls. Additional clinical sample data is shown in FIG. 41A-41F. TLMV: Torque teno-like mini virus; HPV: human papillomavirus; HCV: hepatitis C virus; HBV: hepatitis B virus; HPIV-1: human parainfluenza virus 1; HIV: human immunodeficiency virus; B19 virus: parvovirus B19.

FIG. 38A-38G Design and characterization of 1,050 color codes. FIG. 38A Design of 1,050 color codes. FIG. 38B Schematic for characterization of 210 color codes and the 3-color dimension of 1,050 color codes. FIG. 38C Raw data from characterization of 210 color codes. FIG. 38D Performance of 210 color codes in 3-color space. FIG. 38E Performance of 1,050 color codes in 3-color space. FIG. 38F Illustration of the sliding distance filter (circle) in 3-color space. FIG. 38G Characterization schematic and performance of 1,050 color codes in the 4th color dimension.

FIG. 39A-39G Human associated virus (HAV) panel design schematic and statistics. FIG. 39A there are 576 human-associated viral species with at least 1 genome neighbor in NCBI, and 169 with ≥10 genome neighbors. Genomes were aligned by segment and analyzed the sequence diversity using CATCH-dx to determine optimal primer and crRNA binding sites (see Methods for details). FIG. 39B Number of species in each family in the human-associated virus panel design. FIG. 39C Number of primer pairs required to capture at least 90% of the sequence diversity within each species. Two species required the use of primer pairs containing degenerate bases FIG. 39D Number of crRNAs required to capture at least 90% of the sequence diversity within each species. FIG. 39E The fraction of sequences within each species covered by each designed crRNA set; small crRNA sets were designed with 90% or greater coverage for 164 of the 169 species. To compare expected and observed performance for the HAV panel, FIG. 39F primers and FIG. 39G crRNAs were classified into on-target, low activity, or cross-reactive by sequence analysis (blue or black) or based on experimental data (orange).

FIG. 40A-40E crRNA performance during human-associated virus panel testing. FIG. 40A Individual guide performance for rounds 1 and 2. Redesign and redilution between rounds of testing are indicated between the data from rounds 1 and 2. “On-target”: reactivity above threshold for intended target only. “Cross-reactive”: off-target reactivity above threshold. “Low activity”: no reactivity above threshold. FIG. 40B Summary bar graph of crRNA performance in rounds 1 and 2. FIG. 40C Summary table of redesign, redilution, and concordance between rounds 1 and 2 for unchanged tests. FIG. 40D Round 1 and FIG. 40E round 2 ranked areas under the curve (AUC) for receiver operating characteristics for on-target vs off-target reactivity in round 1 of testing. Representative on-target and off-target distributions are shown for the indicated ranks.

FIG. 41A-41F Synthetic target and clinical sample testing with HAV panel. FIG. 41A Sample handling and data analysis for unknown samples. Following multiplexed PCR with 15 pools, PCR products are combined into sets of 3. A subset of the crRNAs correspond to the primers in each PCR product pool, shown by the colors in the expanded heatmap. Composite heatmaps are generated by combining data from the PCR product pools in the expanded heatmap. FIG. 41B Five synthetic targets (104 cp/μl) were amplified with all primer pools and detected using 169 crRNAs from the HAV panel plus HCV crRNA 2. Controls were the same as those shown in c. FIG. 41C 4 HCV and 4 HIV clinical samples were tested using the HAV 10 panel plus HCV crRNA 2, shown as composite heatmaps. FIG. 41D 986 Reactivity of the same samples from FIG. 41C with just the HCV crRNAs, shown at 1 and 3 hours. FIG. 41E Comparison of PCR amplification scores and CARMEN fluorescence for a subset of viruses from the dengue, Zika, and healthy samples displayed in FIG. 37. FIG. 41F Comparison of PCR amplification scores and CARMEN fluorescence for a subset of viruses from the HIV, HCV, and healthy samples displayed in FIG. 41C. CARMEN fluorescence is background subtracted fluorescence after 1 hour, except HCV crRNA2, which is after 3 hours. Heatmaps indicate background-subtracted fluorescence after 1 hour unless otherwise noted. TLMV: Torque teno-like minivirus; HPV: human papillomavirus; HCV: hepatitis C virus; HBV: hepatitis B virus; HPIV-1: human parainfluenza virus 1; HIV: human immunodeficiency virus; B19 virus: parvovirus B19.

FIG. 42A-42C Performance of Influenza A subtyping and HIV reverse transcriptase (RT) mutation detection. FIG. 42A Distributions of droplet fluorescence for each influenza H-subtype crRNA with each target. A receiver operating characteristic (ROC) curve for on-target reactivity (e.g. crRNA H1 with Target H1) vs all off-target activity (e.g. crRNA H1 with any other target) is shown. FIG. 42B Heatmap showing the full set of crRNAs designed to capture influenza N sequence diversity. 35 synthetic targets (104 cp/μl) were tested using 35 crRNAs. Gray: below detection threshold; Green: fluorescence counts above threshold; Orange outlines: subtypes; Lowest row displays which targets are detected. FIG. 42C Distributions of droplet fluorescence for each HIV RT crRNA-target pair after 30 min in most cases; 3 hour time point for V106M and M184V. SNP indices in FIG. 4B are calculated from the medians of these distributions.

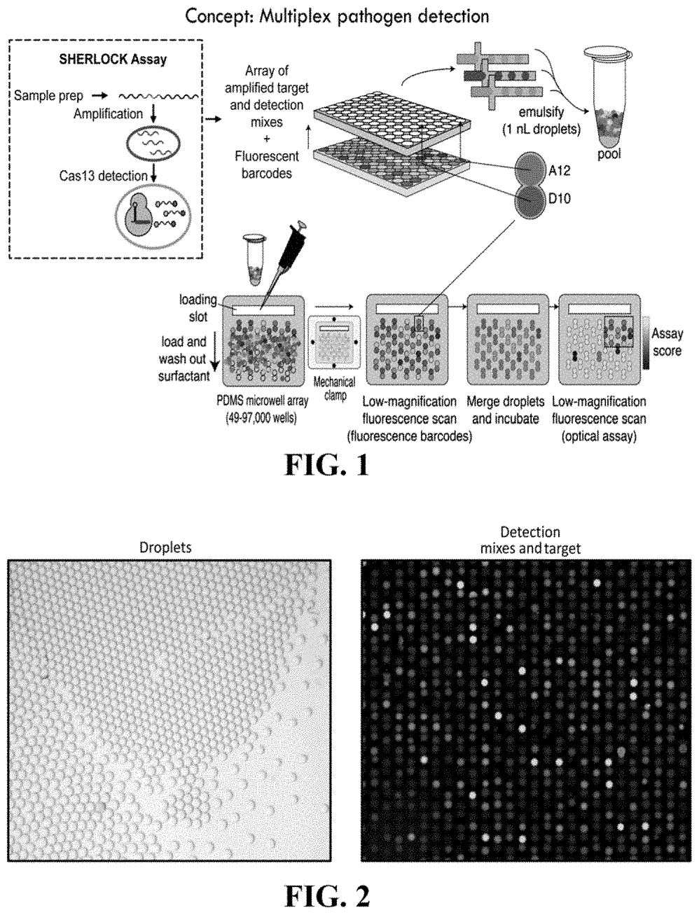

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

“C2c2” is now referred to as “Cas13a”, and the terms are used interchangeably herein unless indicated otherwise.

All publications, published patent documents, and patent applications cited herein are hereby incorporated by reference to the same extent as though each individual publication, published patent document, or patent application was specifically and individually indicated as being incorporated by reference.

### Overview

The embodiments disclosed herein utilize RNA targeting proteins to provide a robust CRISPR-based diagnostic for massively multiplexed applications by performing detection in droplets. Embodiments disclosed herein can detect both DNA and RNA with comparable levels of sensitivity and can differentiate targets from non-targets based on single base pair differences at nanoliter volumes. Such embodiments are useful in multiple scenarios in human health including, for example, viral detection, bacterial strain typing, sensitive genotyping, multiplexed SNP detection, multiplexed strain discrimination and detection of disease-associated cell free DNA. For ease of reference, the embodiments disclosed herein may also be referred to as SHERLOCK (Specific High-sensitivity Enzymatic Reporter unLOCKing), which, in some embodiments, is performed in droplets that can be multiplexed, advantageously allowing sensitive detection with small volumes.

The presently disclosed subject matter utilizes programmable endonucleases, including single RNA-guided RNases (Shmakov et al., 2015; Abudayyeh et al., 2016; Smargon et al., 2017), including C2c2 to provide a platform for specific RNA sensing. The RNA-guided RNA endonucleases from Microbial Clustered Regularly Interspaced Short Palindromic Repeats (CRISPR) and CRISPR-associated (CRISPR-Cas) adaptive immune systems can be easily and conveniently reprogrammed using CRISPR RNA (crRNAs) to cleave target RNAs. RNA-guided RNases, like C2c2, remains active after cleaving its RNA target, leading to “collateral” cleavage of non-targeted RNAs in proximity (Abudayyeh et al., 2016). This crRNA-programmed collateral RNA cleavage activity presents the opportunity to use RNA-guided RNases to detect the presence of a specific RNA by triggering in vivo programmed cell death or in vitro nonspecific RNA degradation that can serve as a readout (Abudayyeh et al., 2016; East-Seletsky et al., 2016). The presently disclosed subject matter utilizes the cleavage activity in a droplet application to enable multiplexed reactions with small volume samples.

In one aspect a multiplex detection system is provided, which comprises a detection CRISPR system; optical barcodes for one or more target molecules, and a microfluidic device. In some embodiments, the detection CRISPR system comprises an RNA targeting effector protein, one or more guide RNAs designed to bind to corresponding target molecules, an RNA based masking construct, and an optical barcode. In some embodiments, the microfluidic device comprises an array of microwells and at least one flow channel beneath the microwells, with the microwells sized to capture at least two droplets. The system can be provided as a kit.

In an aspect, the embodiments disclosed herein are directed to methods for detecting target nucleic acids in a sample. The methods disclosed herein can, in some embodiments, comprise steps of generating a first set of droplets, each droplet in the first set of droplets comprising at least one target molecule and an optical barcode; generating a second set of droplets, each droplet in the second set of droplets comprising a detection CRISPR system comprising an RNA targeting effector protein and one or more guide RNAs designed to bind to corresponding target molecules, an RNA-based masking construct and optionally an optical barcode; combining the first set and second set of droplets into a pool of droplets and flowing the combined pool of droplets onto a microfluidic device comprising an array of microwells and at least one flow channel beneath the microwells, the microwells sized to capture at least two droplets; capturing droplets in the microwell and detecting the optical barcodes of the droplets captured in each microwell; merging the droplets captured in each microwell to formed merged droplets in each microwell, at least a subset of the merged droplets comprising a detection CRISPR system and a target sequence; initiating the detection reaction. The merged droplets are then maintained under conditions sufficient to allow binding of the one or more guide RNAs to one or more target molecules. Binding of the one or more guide RNAs to a target nucleic acid in turn activates the CRISPR effector protein. Once activated, the CRISPR effector protein then deactivates the masking construct, for example, by cleaving the masking construct such that a detectable positive signal is unmasked, released, or generated. Detection and measuring a detectable signal of each merged droplet at one or more time periods can be performed, indicating the presence of target molecules when, for example the positive detectable signal is present.

In particular embodiments, the systems are highly targeted for single samples such that an optical barcode in a second set of barcodes is not needed, or is optional. In certain embodiments, advanced, improved, or more powerful preamplification methods allow omission of an optical barcode in a set of the droplets. Accordingly, optical barcodes in a set of droplets are optional, and inclusion can depend on the particular application, including sample quality, target specificity, preamplification techniques, among other variables.

### Multiplex Detection System

Multiplex systems are disclosed and include a detection CRISPR system comprising an RNA targeting effector protein and one or more guide RNAs designed to bind to corresponding target molecules, an RNA-based masking construct and an optical barcode; one or more target molecule optical barcodes; and a microfluidic device comprising an array of microwells and at least one flow channel beneath the microwells. In embodiments, the microwells are sized to capture at least two droplets.

In general, a CRISPR-Cas or CRISPR system as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system).

### RNA Targeting Cas Protein

When the Cas protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023., which is incorporated herein in its entirety by reference. CRISPR effector proteins described in International Application No. PCT/US2017/065477, Tables 1-6, pages 40-52, can be used in the presently disclosed methods, systems and devices, and are specifically incorporated herein by reference.

The two or more CRISPR systems may be RNA-targeting proteins, DNA-targeting effector proteins, or a combination thereof. The RNA-targeting proteins may be a Cas13 protein, such as Cas13a, Cas13b, or Cas13c. The DNA-targeting protein may be a Cas12 protein such as Cpf1 and C2c1.

### Cpf1 Orthologs

The present invention encompasses the use of a Cpf1 effector protein, derived from a Cpf1 locus denoted as subtype V-A. Herein such effector proteins are also referred to as “Cpf1p”, e.g., a Cpf1 protein (and such effector protein or Cpf1 protein or protein derived from a Cpf1 locus is also called “CRISPR enzyme”). Presently, the subtype V-A loci encompasses cas1, cas2, a distinct gene denoted cpf1 and a CRISPR array. Cpf1 (CRISPR-associated protein Cpf1, subtype PREFRAN) is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in all Cas9 proteins, and the RuvC-like domain is contiguous in the Cpf1 sequence, in contrast to Cas9 where it contains long inserts including the HNH domain. Accordingly, in particular embodiments, the CRISPR-Cas enzyme comprises only a RuvC-like nuclease domain.

The programmability, specificity, and collateral activity of the RNA-guided Cpf1 also make it an ideal switchable nuclease for non-specific cleavage of nucleic acids. In one embodiment, a Cpf1 system is engineered to provide and take advantage of collateral non-specific cleavage of RNA. In another embodiment, a Cpf1 system is engineered to provide and take advantage of collateral non-specific cleavage of ssDNA. Accordingly, engineered Cpf1 systems provide platforms for nucleic acid detection and transcriptome manipulation. Cpf1 is developed for use as a mammalian transcript knockdown and binding tool. Cpf1 is capable of robust collateral cleavage of RNA and ssDNA when activated by sequence-specific targeted DNA binding.

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225.). See also Shmakov et al. (2015) for application in the field of CRISPR-Cas loci. Homologous proteins may but need not be structurally related, or are only partially structurally related.

The Cpf1 gene is found in several diverse bacterial genomes, typically in the same locus with cas1, cas2, and cas4 genes and a CRISPR cassette (for example, FNFX1_1431-FNFX1_1428 of Francisella cf. novicida Fx1). Thus, the layout of this putative novel CRISPR-Cas system appears to be similar to that of type II-B. Furthermore, similar to Cas9, the Cpf1 protein contains a readily identifiable C-terminal region that is homologous to the transposon ORF-B and includes an active RuvC-like nuclease, an arginine-rich region, and a Zn finger (absent in Cas9). However, unlike Cas9, Cpf1 is also present in several genomes without a CRISPR-Cas context and its relatively high similarity with ORF-B suggests that it might be a transposon component. It was suggested that if this was a genuine CRISPR-Cas system and Cpf1 is a functional analog of Cas9 it would be a novel CRISPR-Cas type, namely type V (See Annotation and Classification of CRISPR-Cas Systems. Makarova K S, Koonin E V. Methods Mol Biol. 2015; 1311:47-75). However, as described herein, Cpf1 is denoted to be in subtype V-A to distinguish it from C2c1p which does not have an identical domain structure and is hence denoted to be in subtype V-B.

In particular embodiments, the effector protein is a Cpf1 effector protein from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus.

In further particular embodiments, the Cpf1 effector protein is from an organism selected from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii.

The effector protein may comprise a chimeric effector protein comprising a first fragment from a first effector protein (e.g., a Cpf1) ortholog and a second fragment from a second effector (e.g., a Cpf1) protein ortholog, and wherein the first and second effector protein orthologs are different. At least one of the first and second effector protein (e.g., a Cpf1) orthologs may comprise an effector protein (e.g., a Cpf1) from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus; e.g., a chimeric effector protein comprising a first fragment and a second fragment wherein each of the first and second fragments is selected from a Cpf1 of an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus wherein the first and second fragments are not from the same bacteria; for instance a chimeric effector protein comprising a first fragment and a second fragment wherein each of the first and second fragments is selected from a Cpf1 of S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC20171, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae, wherein the first and second fragments are not from the same bacteria.

In a more preferred embodiment, the Cpf1p is derived from a bacterial species selected from Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cpf1p is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida.

In some embodiments, the Cpf1p is derived from an organism from the genus of Eubacterium. In some embodiments, the CRISPR effector protein is a Cpf1 protein derived from an organism from the bacterial species of Eubacterium rectale. In some embodiments, the amino acid sequence of the Cpf1 effector protein corresponds to NCBI Reference Sequence WP_055225123.1, NCBI Reference Sequence WP_055237260.1, NCBI Reference Sequence WP_055272206.1, or GenBank ID OLA16049.1. In some embodiments, the Cpf1 effector protein has a sequence homology or sequence identity of at least 60%, more particularly at least 70, such as at least 80%, more preferably at least 85%, even more preferably at least 90%, such as for instance at least 95%, with NCBI Reference Sequence WP_055225123.1, NCBI Reference Sequence WP_055237260.1, NCBI Reference Sequence WP_055272206.1, or GenBank ID OLA16049.1. The skilled person will understand that this includes truncated forms of the Cpf1 protein whereby the sequence identity is determined over the length of the truncated form. In some embodiments, the Cpf1 effector recognizes the PAM sequence of TTTN or CTTN.

In particular embodiments, the homologue or orthologue of Cpf1 as referred to herein has a sequence homology or identity of at least 80%, more preferably at least 85%, even more preferably at least 90%, such as for instance at least 95% with Cpf1. In further embodiments, the homologue or orthologue of Cpf1 as referred to herein has a sequence identity of at least 80%, more preferably at least 85%, even more preferably at least 90%, such as for instance at least 95% with the wild type Cpf1. Where the Cpf1 has one or more mutations (mutated), the homologue or orthologue of said Cpf1 as referred to herein has a sequence identity of at least 80%, more preferably at least 85%, even more preferably at least 90%, such as for instance at least 95% with the mutated Cpf1.

In an embodiment, the Cpf1 protein may be an ortholog of an organism of a genus which includes, but is not limited to Acidaminococcus sp, Lachnospiraceae bacterium or Moraxella bovoculi; in particular embodiments, the type V Cas protein may be an ortholog of an organism of a species which includes, but is not limited to Acidaminococcus sp. BV3L6; Lachnospiraceae bacterium ND2006 (LbCpf1) or Moraxella bovoculi 237. In particular embodiments, the homologue or orthologue of Cpf1 as referred to herein has a sequence homology or identity of at least 80%, more preferably at least 85%, even more preferably at least 90%, such as for instance at least 95% with one or more of the Cpf1 sequences disclosed herein. In further embodiments, the homologue or orthologue of Cpf as referred to herein has a sequence identity of at least 80%, more preferably at least 85%, even more preferably at least 90%, such as for instance at least 95% with the wild type FnCpf1, AsCpf1 or LbCpf1.

In particular embodiments, the Cpf1 protein of the invention has a sequence homology or identity of at least 60%, more particularly at least 70, such as at least 80%, more preferably at least 85%, even more preferably at least 90%, such as for instance at least 95% with FnCpf1, AsCpf1 or LbCpf1. In further embodiments, the Cpf1 protein as referred to herein has a sequence identity of at least 60%, such as at least 70%, more particularly at least 80%, more preferably at least 85%, even more preferably at least 90%, such as for instance at least 95% with the wild type AsCpf1 or LbCpf1. In particular embodiments, the Cpf1 protein of the present invention has less than 60% sequence identity with FnCpf1. The skilled person will understand that this includes truncated forms of the Cpf1 protein whereby the sequence identity is determined over the length of the truncated form.

In certain of the following, Cpf1 amino acids are followed by nuclear localization signals (NLS) (italics), a glycine-serine (GS) linker, and 3×HA tag. 1—Franscisella tularensis subsp. novicida U112 (FnCpf1); 3—Lachnospiraceae bacterium MC2017 (Lb3Cpf1); 4—Butyrivibrio proteoclasticus (BpCpf1); 5—Peregrinibacteria bacterium GW2011_GWA_33_10 (PeCpf1); 6—Parcubacteria bacterium GWC2011_GWC2_44_17 (PbCpf1); 7—Smithella sp. SC_K08D17 (SsCpf1); 8—Acidaminococcus sp. BV3L6 (AsCpf1); 9—Lachnospiraceae bacterium MA2020 (Lb2Cpf1); 10—Candidatus Methanoplasma termitum (CMtCpf1); 11—Eubacterium eligens (EeCpf1); 12—Moraxella bovoculi 237 (MbCpf1); 13—Leptospira inadai (LiCpf1); 14—Lachnospiraceae bacterium ND2006 (LbCpf1); 15—Porphyromonas crevioricanis (PcCpf1); 16—Prevotella disiens (PdCpf1); 17—Porphyromonas macacae (PmCpf1); 18—Thiomicrospira sp. XS5 (TsCpf1); 19—Moraxella bovoculi AAX08_00205 (Mb2Cpf1); 20—Moraxella bovoculi AAX11_00205 (Mb3Cpf1); and 21—Butyrivibrio sp. NC3005 (BsCpf1).

Further Cpf1 orthologs include NCBI WP_055225123.1, NCBI WP_055237260.1, NCBI WP_055272206.1, and GenBank OLA16049.1.

### C2c1 Orthologs

The present invention encompasses the use of a C2c1 effector protein, derived from a C2c1 locus denoted as subtype V-B. Herein such effector proteins are also referred to as “C2c1p”, e.g., a C2c1 protein (and such effector protein or C2c1 protein or protein derived from a C2c1 locus is also called “CRISPR enzyme”). Presently, the subtype V-B loci encompasses cas1-Cas4 fusion, cas2, a distinct gene denoted C2c1 and a CRISPR array. C2c1 (CRISPR-associated protein C2c1) is a large protein (about 1100-1300 amino acids) that contains a RuvC-like nuclease domain homologous to the corresponding domain of Cas9 along with a counterpart to the characteristic arginine-rich cluster of Cas9. However, C2c1 lacks the HNH nuclease domain that is present in all Cas9 proteins, and the RuvC-like domain is contiguous in the C2c1 sequence, in contrast to Cas9 where it contains long inserts including the HNH domain. Accordingly, in particular embodiments, the CRISPR-Cas enzyme comprises only a RuvC-like nuclease domain.

C2c1 (also known as Cas12b) proteins are RNA guided nucleases. Its cleavage relies on a tracr RNA to recruit a guide RNA comprising a guide sequence and a direct repeat, where the guide sequence hybridizes with the target nucleotide sequence to form a DNA/RNA heteroduplex. Based on current studies, C2c1 nuclease activity also requires relies on recognition of PAM sequence. C2c1 PAM sequences are T-rich sequences. In some embodiments, the PAM sequence is 5′ TTN 3′ or 5′ ATTN 3′, wherein N is any nucleotide. In a particular embodiment, the PAM sequence is 5′ TTC 3′. In a particular embodiment, the PAM is in the sequence of Plasmodium falciparum.

C2c1 creates a staggered cut at the target locus, with a 5′ overhang, or a “sticky end” at the PAM distal side of the target sequence. In some embodiments, the 5′ overhang is 7 nt. See Lewis and Ke, Mol Cell. 2017 Feb. 2; 65(3):377-379.

The invention provides C2c1 (Type V-B; Cas12b) effector proteins and orthologues. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225.). See also Shmakov et al. (2015) for application in the field of CRISPR-Cas loci. Homologous proteins may but need not be structurally related, or are only partially structurally related.

The C2c1 gene is found in several diverse bacterial genomes, typically in the same locus with cas1, cas2, and cas4 genes and a CRISPR cassette. Thus, the layout of this putative novel CRISPR-Cas system appears to be similar to that of type II-B. Furthermore, similar to Cas9, the C2c1 protein contains an active RuvC-like nuclease, an arginine-rich region, and a Zn finger (absent in Cas9).

In particular embodiments, the effector protein is a C2c1 effector protein from an organism from a genus comprising Alicyclobacillus, Desulfovibrio, Desulfonatronum, Opitutaceae, Tuberibacillus, Bacillus, Brevibacillus, Candidatus, Desulfatirhabdium, Citrobacter, Elusimicrobia, Methylobacterium, Omnitrophica, Phycisphaerae, Planctomycetes, Spirochaetes, and Verrucomicrobiaceae.

In further particular embodiments, the C2c1 effector protein is from a species selected from Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060).

The effector protein may comprise a chimeric effector protein comprising a first fragment from a first effector protein (e.g., a C2c1) ortholog and a second fragment from a second effector (e.g., a C2c1) protein ortholog, and wherein the first and second effector protein orthologs are different. At least one of the first and second effector protein (e.g., a C2c1) orthologs may comprise an effector protein (e.g., a C2c1) from an organism comprising Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae; e.g., a chimeric effector protein comprising a first fragment and a second fragment wherein each of the first and second fragments is selected from a C2c1 of an organism comprising Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae wherein the first and second fragments are not from the same bacteria; for instance a chimeric effector protein comprising a first fragment and a second fragment wherein each of the first and second fragments is selected from a C2c1 of Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060), wherein the first and second fragments are not from the same bacteria.

In a more preferred embodiment, the C2c1p is derived from a bacterial species selected from Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060). In certain embodiments, the C2c1p is derived from a bacterial species selected from Alicyclobacillus acidoterrestris (e.g., ATCC 49025), Alicyclobacillus contaminans (e.g., DSM 17975).

In particular embodiments, the homologue or orthologue of C2c1 as referred to herein has a sequence homology or identity of at least 80%, more preferably at least 85%, even more preferably at least 90%, such as for instance at least 95% with C2c1. In further embodiments, the homologue or orthologue of C2c1 as referred to herein has a sequence identity of at least 80%, more preferably at least 85%, even more preferably at least 90%, such as for instance at least 95% with the wild type C2c1. Where the C2c1 has one or more mutations (mutated), the homologue or orthologue of said C2c1 as referred to herein has a sequence identity of at least 80%, more preferably at least 85%, even more preferably at least 90%, such as for instance at least 95% with the mutated C2c1.

In an embodiment, the C2c1 protein may be an ortholog of an organism of a genus which includes, but is not limited to Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae; in particular embodiments, the type V Cas protein may be an ortholog of an organism of a species which includes, but is not limited to Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF 112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060). In particular embodiments, the homologue or orthologue of C2c1 as referred to herein has a sequence homology or identity of at least 80%, more preferably at least 85%, even more preferably at least 90%, such as for instance at least 95% with one or more of the C2c1 sequences disclosed herein. In further embodiments, the homologue or orthologue of C2c1 as referred to herein has a sequence identity of at least 80%, more preferably at least 85%, even more preferably at least 90%, such as for instance at least 95% with the wild type AacC2c1 or BthC2c1.

In particular embodiments, the C2c1 protein of the invention has a sequence homology or identity of at least 60%, more particularly at least 70, such as at least 80%, more preferably at least 85%, even more preferably at least 90%, such as for instance at least 95% with AacC2c1 or BthC2c1. In further embodiments, the C2c1 protein as referred to herein has a sequence identity of at least 60%, such as at least 70%, more particularly at least 80%, more preferably at least 85%, even more preferably at least 90%, such as for instance at least 95% with the wild type AacC2c1. In particular embodiments, the C2c1 protein of the present invention has less than 60% sequence identity with AacC2c1. The skilled person will understand that this includes truncated forms of the C2c1 protein whereby the sequence identity is determined over the length of the truncated form.

In certain methods according to the present invention, the CRISPR-Cas protein is preferably mutated with respect to a corresponding wild-type enzyme such that the mutated CRISPR-Cas protein lacks the ability to cleave one or both DNA strands of a target locus containing a target sequence. In particular embodiments, one or more catalytic domains of the C2c1 protein are mutated to produce a mutated Cas protein which cleaves only one DNA strand of a target sequence.

In particular embodiments, the CRISPR-Cas protein may be mutated with respect to a corresponding wild-type enzyme such that the mutated CRISPR-Cas protein lacks substantially all DNA cleavage activity. In some embodiments, a CRISPR-Cas protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form.

In certain embodiments of the methods provided herein the CRISPR-Cas protein is a mutated CRISPR-Cas protein which cleaves only one DNA strand, i.e. a nickase. More particularly, in the context of the present invention, the nickase ensures cleavage within the non-target sequence, i.e. the sequence which is on the opposite DNA strand of the target sequence and which is 3′ of the PAM sequence. By means of further guidance, and without limitation, an arginine-to-alanine substitution (R911A) in the Nuc domain of C2c1 from Alicyclobacillus acidoterrestris converts C2c1 from a nuclease that cleaves both strands to a nickase (cleaves a single strand). It will be understood by the skilled person that where the enzyme is not AacC2c1, a mutation may be made at a residue in a corresponding position.

In certain embodiments, the C2c1 protein is a catalytically inactive C2c1 which comprises a mutation in the RuvC domain. In some embodiments, the catalytically inactive C2c1 protein comprises a mutation corresponding to amion acid positions D570, E848, or D977 in Alicyclobacillus acidoterrestris C2c1. In some embodiments, the catalytically inactive C2c1 protein comprises a mutation corresponding to D570A, E848A, or D977A in Alicyclobacillus acidoterrestris C2c1.

The programmability, specificity, and collateral activity of the RNA-guided C2c1 also make it an ideal switchable nuclease for non-specific cleavage of nucleic acids. In one embodiment, a C2c1 system is engineered to provide and take advantage of collateral non-specific cleavage of RNA. In another embodiment, a C2c1 system is engineered to provide and take advantage of collateral non-specific cleavage of ssDNA. Accordingly, engineered C2c1 systems provide platforms for nucleic acid detection and transcriptome manipulation, and inducing cell death. C2c1 is developed for use as a mammalian transcript knockdown and binding tool. C2c1 is capable of robust collateral cleavage of RNA and ssDNA when activated by sequence-specific targeted DNA binding.

In certain embodiments, C2c1 is provided or expressed in an in vitro system or in a cell, transiently or stably, and targeted or triggered to non-specifically cleave cellular nucleic acids. In one embodiment, C2c1 is engineered to knock down ssDNA, for example viral ssDNA. In another embodiment, C2c1 is engineered to knock down RNA. The system can be devised such that the knockdown is dependent on a target DNA present in the cell or in vitro system, or triggered by the addition of a target nucleic acid to the system or cell.

In an embodiment, the C2c1 system is engineered to non-specifically cleave RNA in a subset of cells distinguishable by the presence of an aberrant DNA sequence, for instance where cleavage of the aberrant DNA might be incomplete or ineffectual. In one non-limiting example, a DNA translocation that is present in a cancer cell and drives cell transformation is targeted. Whereas a subpopulation of cells that undergoes chromosomal DNA and repair may survive, non-specific collateral ribonuclease activity advantageously leads to cell death of potential survivors.

Collateral activity was recently leveraged for a highly sensitive and specific nucleic acid detection platform termed SHERLOCK that is useful for many clinical diagnoses (Gootenberg, J. S. et al. Nucleic acid detection with CRISPR-Cas13a/C2c2. Science 356, 438-442 (2017)).

According to the invention, engineered C2c1 systems are optimized for DNA or RNA endonuclease activity and can be expressed in mammalian cells and targeted to effectively knock down reporter molecules or transcripts in cells.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U. In certain embodiments, the effector protein may be Leptotrichia shahii C2c2p, more preferably Leptotrichia shahii DSM 19757 C2c2, and the 3′ PAM is a 5′ H.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

The nucleic acid molecule encoding a CRISPR effector protein, in particular C2c2, is advantageously codon optimized CRISPR effector protein. An example of a codon optimized sequence is in this instance a sequence optimized for expression in eukaryotes, e.g., humans (i.e. being optimized for expression in humans), or for another eukaryote, animal or mammal as herein discussed; see, e.g., SaCas9 human codon optimized sequence in WO 2014/093622 (PCT/US2013/074667). While this is preferred, it will be appreciated that other examples are possible and codon optimization for a host species other than human, or for codon optimization for specific organs, is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal including, but not limited to, human or non-human eukaryote, or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell, in particular a C2c2 transgenic cell, in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). As used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regard to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words, samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well-established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the 3-actin promoter, the phosphoglycerol kinase (PGK) promoter, and the EF1α promoter. An advantageous promoter is the promoter U6.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains. The skilled person will understand that truncated forms of the C2c2 proteins can be utilized, whereby the sequence identity is determined over the length of the truncated form.

In one example embodiment, the effector protein comprises one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in PCT/US2017/038154 entitled “Novel Type VI CRISPR Orthologs and Systems,” at, for example, pages 256-264 and 285-336, U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application 62/484,786 entitled “Novel Type VI CRISPR Orthologs and Systems,” filed on Apr. 12, 2017.

In an embodiment of the invention, a HEPN domain comprises at least one RxxxxH motif comprising the sequence of R{N/H/K}X1X2X3H (SEQ ID NO:1). In an embodiment of the invention, a HEPN domain comprises a RxxxxH motif comprising the sequence of R{N/H}X1X2X3H (SEQ ID NO:2). In an embodiment of the invention, a HEPN domain comprises the sequence of R{N/K}X1X2X3H (SEQ ID NO:3). In certain embodiments, X1 is R, S, D, E, Q, N, G, Y, or H. In certain embodiments, X2 is I, S, T, V, or L. In certain embodiments, X3 is L, F, N, Y, V, I, S, D, E, or A.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

In particular embodiments, the Type VI RNA-targeting Cas enzyme is C2c2. In other example embodiments, the Type VI RNA-targeting Cas enzyme is Cas 13b. In particular embodiments, the homologue or orthologue of a Type VI protein such as C2c2 as referred to herein has a sequence homology or identity of at least 30%, or at least 40%, or at least 50%, or at least 60%, or at least 70%, or at least 80%, more preferably at least 85%, even more preferably at least 90%, such as for instance at least 95% with a Type VI protein such as C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2). In further embodiments, the homologue or orthologue of a Type VI protein such as C2c2 as referred to herein has a sequence identity of at least 30%, or at least 40%, or at least 50%, or at least 60%, or at least 70%, or at least 80%, more preferably at least 85%, even more preferably at least 90%, such as for instance at least 95% with the wild type C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2).

In certain other example embodiments, the CRISPR system the effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to International Patent Publication WO/2017/219027, entitled TYPE VI CRISPR ORTHOLOGS AND SYSTEMS, U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

RNase function in CRISPR systems is known, for example mRNA targeting has been reported for certain type III CRISPR-Cas systems (Hale et al., 2014, Genes Dev, vol. 28, 2432-2443; Hale et al., 2009, Cell, vol. 139, 945-956; Peng et al., 2015, Nucleic acids research, vol. 43, 406-417) and provides significant advantages. In the Staphylococcus epidermis type III-A system, transcription across targets results in cleavage of the target DNA and its transcripts, mediated by independent active sites within the Cas10-Csm ribonucleoprotein effector protein complex (see, Samai et al., 2015, Cell, vol. 151, 1164-1174). A CRISPR-Cas system, composition or method targeting RNA via the present effector proteins is thus provided.

In an embodiment, the Cas protein may be a C2c2 ortholog of an organism of a genus which includes but is not limited to Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira. Species of organism of such a genus can be as otherwise herein discussed.

In certain example embodiments, the C2c2 effector proteins of the invention include, without limitation, the following 21 ortholog species (including multiple CRISPR loci: Leptotrichia shahii; Leptotrichia wadei (Lw2); Listeria seeligeri; Lachnospiraceae bacterium MA2020; Lachnospiraceae bacterium NK4A179; [Clostridium] aminophilum DSM 10710; Carnobacterium gallinarum DSM 4847; Carnobacterium gallinarum DSM 4847 (second CRISPR Loci); Paludibacter propionicigenes WB4; Listeria weihenstephanensis FSL R9-0317; Listeriaceae bacterium FSL M6-0635; Leptotrichia wadei F0279; Rhodobacter capsulatus SB 1003; Rhodobacter capsulatus R121; Rhodobacter capsulatus DE442; Leptotrichia buccalis C-1013-b; Herbinix hemicellulosilytica; [Eubacterium] rectale; Eubacteriaceae bacterium CHKCI004; Blautia sp. Marseille-P2398; and Leptotrichia sp. oral taxon 879 str. F0557. Twelve (12) further non-limiting examples are: Lachnospiraceae bacterium NK4A144; Chloroflexus aggregans; Demequina aurantiaca; Thalassospira sp. TSL5-1; Pseudobutyrivibrio sp. OR37; Butyrivibrio sp. YAB3001; Blautia sp. Marseille-P2398; Leptotrichia sp. Marseille-P3007; Bacteroides ihuae; Porphyromonadaceae bacterium KH3CP3RA; Listeria riparia; and Insolitispirillum peregrinum.

Some methods of identifying orthologues of CRISPR-Cas system enzymes may involve identifying tracr sequences in genomes of interest. Identification of tracr sequences may relate to the following steps: Search for the direct repeats or tracr mate sequences in a database to identify a CRISPR region comprising a CRISPR enzyme. Search for homologous sequences in the CRISPR region flanking the CRISPR enzyme in both the sense and antisense directions. Look for transcriptional terminators and secondary structures. Identify any sequence that is not a direct repeat or a tracr mate sequence but has more than 50% identity to the direct repeat or tracr mate sequence as a potential tracr sequence. Take the potential tracr sequence and analyze for transcriptional terminator sequences associated therewith.

It will be appreciated that any of the functionalities described herein may be engineered into CRISPR enzymes from other orthologs, including chimeric enzymes comprising fragments from multiple orthologs. Examples of such orthologs are described elsewhere herein. Thus, chimeric enzymes may comprise fragments of CRISPR enzyme orthologs of an organism which includes but is not limited to Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter. A chimeric enzyme can comprise a first fragment and a second fragment, and the fragments can be of CRISPR enzyme orthologs of organisms of genera herein mentioned or of species herein mentioned; advantageously the fragments are from CRISPR enzyme orthologs of different species.

In embodiments, the C2c2 protein as referred to herein also encompasses a functional variant of C2c2 or a homologue or an orthologue thereof. A “functional variant” of a protein as used herein refers to a variant of such protein which retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made. Advantageous embodiments can involve engineered or non-naturally occurring Type VI RNA-targeting effector protein.

In an embodiment, nucleic acid molecule(s) encoding the C2c2 or an ortholog or homolog thereof, may be codon-optimized for expression in a eukaryotic cell. A eukaryote can be as herein discussed. Nucleic acid molecule(s) can be engineered or non-naturally occurring.

In an embodiment, the C2c2 or an ortholog or homolog thereof, may comprise one or more mutations (and hence nucleic acid molecule(s) coding for same may have mutation(s). The mutations may be artificially introduced mutations and may include but are not limited to one or more mutations in a catalytic domain. Examples of catalytic domains with reference to a Cas9 enzyme may include but are not limited to RuvC I, RuvC II, RuvC III and HNH domains.

In an embodiment, the C2c2 or an ortholog or homolog thereof, may comprise one or more mutations. The mutations may be artificially introduced mutations and may include but are not limited to one or more mutations in a catalytic domain. Examples of catalytic domains with reference to a Cas enzyme may include but are not limited to HEPN domains.

In an embodiment, the C2c2 or an ortholog or homolog thereof, may be used as a generic nucleic acid binding protein with fusion to or being operably linked to a functional domain. Exemplary functional domains may include but are not limited to translational initiator, translational activator, translational repressor, nucleases, in particular ribonucleases, a spliceosome, beads, a light inducible/controllable domain or a chemically inducible/controllable domain.

In certain example embodiments, the C2c2 effector protein may be from an organism selected from the group consisting of Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, and Campylobacter.

In certain embodiments, the effector protein may be a Listeria sp. C2c2p, preferably Listeria seeligeria C2c2p, more preferably Listeria seeligeria serovar 1/2b str. SLCC3954 C2c2p and the crRNA sequence may be 44 to 47 nucleotides in length, with a 5′ 29-nt direct repeat (DR) and a 15-nt to 18-nt spacer.

In certain embodiments, the effector protein may be a Leptotrichia sp. C2c2p, preferably Leptotrichia shahii C2c2p, more preferably Leptotrichia shahii DSM 19757 C2c2p and the crRNA sequence may be 42 to 58 nucleotides in length, with a 5′ direct repeat of at least 24 nt, such as a 5′ 24-28-nt direct repeat (DR) and a spacer of at least 14 nt, such as a 14-nt to 28-nt spacer, or a spacer of at least 18 nt, such as 19, 20, 21, 22, or more nt, such as 18-28, 19-28, 20-28, 21-28, or 22-28 nt.

In certain example embodiments, the effector protein may be a Leptotrichia sp., Leptotrichia wadei F0279, or a Listeria sp., preferably Listeria newyorkensis FSL M6-0635.

In certain embodiments, the C2c2 protein according to the invention is or is derived from one of the orthologues or is a chimeric protein of two or more of the orthologues as described in this application, or is a mutant or variant of one of the orthologues (or a chimeric mutant or variant), including dead C2c2, split C2c2, destabilized C2c2, etc. as defined herein elsewhere, with or without fusion with a heterologous/functional domain.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In certain example embodiments, different orthologues from a same class of CRISPR effector protein may be used, such as two Cas13a orthologues, two Cas13b orthologues, or two Cas13c orthologues, which is described in International Application No. PCT/US2017/065477, Tables 1-6, pages 40-52, and incorporated herein by reference. On certain other example embodiments, different orthologues with different nucleotide editing preferences may be used such as a Cas13a and Cas13b orthologs, or a Cas13a and a Cas13c orthologs, or a Cas13b orthologs and a Cas13c orthologs etc.

The RNA targeting effector protein can, in some embodiments, comprise one or more HEPN domains, which can optionally comprise a RxxxxH motif sequence. In some instances, the RxxxH motif comprises a R{N/H/K]X₁X₂X₃H sequence, which in some embodiments X₁ is R, S, D, E, Q, N, G, or Y, and X₂ is independently I, S, T, V, or L, and X₃ is independently L, F, N, Y, V, I, S, D, E, or A. In some particular embodiments, the CRISPR RNA-targeting effector protein is C2c2.

Non-specific ssDNA and RNA directed proteins will inevitably lead to further and, potentially, improved Cas proteins that demonstrate collateral cleavage and may be used for detection and offer greater breadth for multiplexed detection of nucleic acid targets in amplified and highly sensitive, especially SHERLOCK, diagnostic systems.

### Guides

As used herein, the term “crRNA” or “guide RNA” or “single guide RNA” or “sgRNA” or “one or more nucleic acid components” of a Type V or Type VI CRISPR-Cas locus effector protein comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop corresponds to the tracr mate sequence, and the portion of the sequence 3′ of the loop corresponds to the tracr sequence.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In general, the CRISPR-Cas, CRISPR-Cas9 or CRISPR system may be as used in the foregoing documents, such as WO 2014/093622 (PCT/US2013/074667) and refers collectively to transcripts and other elements involved in the expression of or directing the activity of CRISPR-associated (“Cas”) genes, including sequences encoding a Cas gene, in particular a Cas9 gene in the case of CRISPR-Cas9, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. The section of the guide sequence through which complementarity to the target sequence is important for cleavage activity is referred to herein as the seed sequence. A target sequence may comprise any polynucleotide, such as DNA or RNA polynucleotides. In some embodiments, a target sequence is located in the nucleus or cytoplasm of a cell, and may include nucleic acids in or from mitochondrial, organelles, vesicles, liposomes or particles present within the cell. In some embodiments, especially for non-nuclear uses, NLSs are not preferred. In some embodiments, a CRISPR system comprises one or more nuclear exports signals (NESs). In some embodiments, a CRISPR system comprises one or more NLSs and one or more NESs. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the invention the terms guide sequence and guide RNA, i.e. RNA capable of guiding Cas to a target genomic locus, are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments of CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In particularly preferred embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e. an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

The methods according to the invention as described herein comprehend inducing one or more mutations in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s) or sgRNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 40, 45, 50, 75, 100, 200, 300, 400 or 500 nucleotides at each target sequence of said cell(s) via the guide(s) RNA(s) or sgRNA(s).

For minimization of toxicity and off-target effect, it may be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D10A mutation) can be delivered with a pair of guide RNAs targeting a site of interest. Guide sequences and strategies to minimize toxicity and off-target effects can be as in WO 2014/093622 (PCT/US2013/074667); or, via mutation as herein.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

### Guide Modifications

In certain embodiments, guides of the invention comprise non-naturally occurring nucleic acids and/or non-naturally occurring nucleotides and/or nucleotide analogs, and/or chemically modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, peptide nucleic acids (PNA), or bridged nucleic acids (BNA). Other examples of modified nucleotides include 2′-O-methyl analogs, 2′-deoxy analogs, 2-thiouridine analogs, N6-methyladenosine analogs, or 2′-fluoro analogs. Further examples of modified nucleotides include linkage of chemical moieties at the 2′ position, including but not limited to peptides, nuclear localization sequence (NLS), peptide nucleic acid (PNA), polyethylene glycol (PEG), triethylene glycol, or tetraethyleneglycol (TEG). Further examples of modified bases include, but are not limited to, 2-aminopurine, 5-bromo-uridine, pseudouridine (Ψ), N¹-methylpseudouridine (me¹Ψ), 5-methoxyuridine (5moU), inosine, 7-methylguanosine. Examples of guide RNA chemical modifications include, without limitation, incorporation of 2′-O-methyl (M), 2′-O-methyl-3′-phosphorothioate (MS), phosphorothioate (PS), S-constrained ethyl(cEt), 2′-O-methyl-3′-thioPACE (MSP), or 2′-O-methyl-3′-phosphonoacetate (MP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015; Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., Med Chem Comm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066; Ryan et al., Nucleic Acids Res. (2018) 46(2): 792-803). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodients, a guide comprises ribonucleotides in a region that binds to a target DNA and one or more deoxyribonucletides and/or nucleotide analogs in a region that binds to Cas9, Cpf1, or C2c1. In an embodiment of the invention, deoxyribonucleotides and/or nucleotide analogs are incorporated in engineered guide structures, such as, without limitation, 5′ and/or 3′ end, stem-loop regions, and the seed region. In certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl-3′-phosphorothioate (MS), S-constrained ethyl(cEt), 2′-O-methyl-3′-thioPACE (MSP), or 2′-O-methyl-3′-phosphonoacetate (MP). Such modification can enhance genome editing efficiency (see Hendel et al., Nat. Biotechnol. (2015) 33(9): 985-989; Ryan et al., Nucleic Acids Res. (2018) 46(2): 792-803). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), Rhodamine, peptides, nuclear localization sequence (NLS), peptide nucleic acid (PNA), polyethylene glycol (PEG), triethylene glycol, or tetraethyleneglycol (TEG). In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554). In some embodiments, 3 nucleotides at each of the 3′ and 5′ ends are chemically modified. In a specific embodiment, the modifications comprise 2′-O-methyl or phosphorothioate analogs. In a specific embodiment, 12 nucleotides in the tetraloop and 16 nucleotides in the stem-loop region are replaced with 2′-O-methyl analogs. Such chemical modifications improve in vivo editing and stability (see Finn et al., Cell Reports (2018), 22: 2227-2235). In some embodiments, more than 60 or 70 nucleotides of the guide are chemically modified. In some embodiments, this modification comprises replacement of nucleotides with 2′-O-methyl or 2′-fluoro nucleotide analogs or phosphorothioate (PS) modification of phosphodiester bonds. In some embodiments, the chemical modification comprises 2′-O-methyl or 2′-fluoro modification of guide nucleotides extending outside of the nuclease protein when the CRISPR complex is formed or PS modification of 20 to 30 or more nucleotides of the 3′-terminus of the guide. In a particular embodiment, the chemical modification further comprises 2′-O-methyl analogs at the 5′ end of the guide or 2′-fluoro analogs in the seed and tail regions. Such chemical modifications improve stability to nuclease degradation and maintain or enhance genome-editing activity or efficiency, but modification of all nucleotides may abolish the function of the guide (see Yin et al., Nat. Biotech. (2018), 35(12): 1179-1187). Such chemical modifications may be guided by knowledge of the structure of the CRISPR complex, including knowledge of the limited number of nuclease and RNA 2′-OH interactions (see Yin et al., Nat. Biotech. (2018), 35(12): 1179-1187). In some embodiments, one or more guide RNA nucleotides may be replaced with DNA nucleotides. In some embodiments, up to 2, 4, 6, 8, 10, or 12 RNA nucleotides of the 5′-end tail/seed guide region are replaced with DNA nucleotides. In certain embodiments, the majority of guide RNA nucleotides at the 3′ end are replaced with DNA nucleotides. In particular embodiments, 16 guide RNA nucleotides at the 3′ end are replaced with DNA nucleotides. In particular embodiments, 8 guide RNA nucleotides of the 5′-end tail/seed region and 16 RNA nucleotides at the 3′ end are replaced with DNA nucleotides. In particular embodiments, guide RNA nucleotides that extend outside of the nuclease protein when the CRISPR complex is formed are replaced with DNA nucleotides. Such replacement of multiple RNA nucleotides with DNA nucleotides leads to decreased off-target activity but similar on-target activity compared to an unmodified guide; however, replacement of all RNA nucleotides at the 3′ end may abolish the function of the guide (see Yin et al., Nat. Chem. Biol. (2018) 14, 311-316). Such modifications may be guided by knowledge of the structure of the CRISPR complex, including knowledge of the limited number of nuclease and RNA 2′-OH interactions (see Yin et al., Nat. Chem. Biol. (2018) 14, 311-316).

In one aspect of the invention, the guide comprises a modified crRNA for Cpf1, having a 5′-handle and a guide segment further comprising a seed region and a 3′-terminus. In some embodiments, the modified guide can be used with a Cpf1 of any one of Acidaminococcus sp. BV3L6 Cpf1 (AsCpf1); Francisella tularensis subsp. Novicida U112 Cpf1 (FnCpf1); L. bacterium MC2017 Cpf1 (Lb3Cpf1); Butyrivibrio proteoclasticus Cpf1 (BpCpf1); Parcubacteria bacterium GWC2011_GWC2_44_17 Cpf1 (PbCpf1); Peregrinibacteria bacterium GW2011_GWA_33_10 Cpf1 (PeCpf1); Leptospira inadai Cpf1 (LiCpf1); Smithella sp. SC_K08D17 Cpf1 (SsCpf1); L. bacterium MA2020 Cpf1 (Lb2Cpf1); Porphyromonas crevioricanis Cpf1 (PcCpf1); Porphyromonas macacae Cpf1 (PmCpf1); Candidatus Methanoplasma termitum Cpf1 (CMtCpf1); Eubacterium eligens Cpf1 (EeCpf1); Moraxella bovoculi 237 Cpf1 (MbCpf1); Prevotella disiens Cpf1 (PdCpf1); or L. bacterium ND2006 Cpf1 (LbCpf1).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N¹-methylpseudouridine (me¹Ψ), 5-methoxyuridine (5moU), inosine, 7-methylguanosine, 2′-O-methyl-3′-phosphorothioate (MS), S-constrained ethyl(cEt), phosphorothioate (PS), 2′-O-methyl-3′-thioPACE (MSP), or 2′-O-methyl-3′-phosphonoacetate (MP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In some embodiments, all nucleotides are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 or 10 nucleotides in the 3′-terminus are chemically modified. Such chemical modifications at the 3′-terminus of the Cpf1 CrRNA improve gene cutting efficiency (see Li, et al., Nature Biomedical Engineering, 2017, 1:0066). In a specific embodiment, 5 nucleotides in the 3′-terminus are replaced with 2′-fluoro analogues. In a specific embodiment, 10 nucleotides in the 3′-terminus are replaced with 2′-fluoro analogues. In a specific embodiment, 5 nucleotides in the 3′-terminus are replaced with 2′-O-methyl (M) analogs. In some embodiments, 3 nucleotides at each of the 3′ and 5′ ends are chemically modified. In a specific embodiment, the modifications comprise 2′-O-methyl or phosphorothioate analogs. In a specific embodiment, 12 nucleotides in the tetraloop and 16 nucleotides in the stem-loop region are replaced with 2′-O-methyl analogs. Such chemical modifications improve in vivo editing and stability (see Finn et al., Cell Reports (2018), 22: 2227-2235).

In some embodiments, the loop of the 5′-handle of the guide is modified. In some embodiments, the loop of the 5′-handle of the guide is modified to have a deletion, an insertion, a split, or chemical modifications. In certain embodiments, the loop comprises 3, 4, or 5 nucleotides. In certain embodiments, the loop comprises the sequence of UCUU, UUUU, UAUU, or UGUU. In some embodiments, the guide molecule forms a stemloop with a separate non-covalently linked sequence, which can be DNA or RNA.

### Synthetically Linked Guide

In one aspect, the guide comprises a tracr sequence and a tracr mate sequence that are chemically linked or conjugated via a non-phosphodiester bond. In one aspect, the guide comprises a tracr sequence and a tracr mate sequence that are chemically linked or conjugated via a non-nucleotide loop. In some embodiments, the tracr and tracr mate sequences are joined via a non-phosphodiester covalent linker. Examples of the covalent linker include but are not limited to a chemical moiety selected from the group consisting of carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, the tracr and tracr mate sequences are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, the tracr or tracr mate sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once the tracr and the tracr mate sequences are functionalized, a covalent chemical bond or linkage can be formed between the two oligonucleotides. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, the tracr and tracr mate sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In some embodiments, the tracr and tracr mate sequences can be covalently linked using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8: 570-9; Behlke et al., Oligonucleotides (2008) 18: 305-19; Watts, et al., Drug. Discov. Today (2008) 13: 842-55; Shukla, et al., Chem Med Chem (2010) 5: 328-49.

In some embodiments, the tracr and tracr mate sequences can be covalently linked using click chemistry. In some embodiments, the tracr and tracr mate sequences can be covalently linked using a triazole linker. In some embodiments, the tracr and tracr mate sequences can be covalently linked using Huisgen 1,3-dipolar cycloaddition reaction involving an alkyne and azide to yield a highly stable triazole linker (He et al., Chem Bio Chem (2015) 17: 1809-1812; WO 2016/186745). In some embodiments, the tracr and tracr mate sequences are covalently linked by ligating a 5′-hexyne tracrRNA and a 3′-azide crRNA. In some embodiments, either or both of the 5′-hexyne tracrRNA and a 3′-azide crRNA can be protected with 2′-acetoxyethl orthoester (2′-ACE) group, which can be subsequently removed using Dharmacon protocol (Scaringe et al., J. Am. Chem. Soc. (1998) 120: 11820-11821; Scaringe, Methods Enzymol. (2000) 317: 3-18).

In some embodiments, the tracr and tracr mate sequences can be covalently linked via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this invention include, but are not limited to, polyethers (e.g., polyethylene glycols, polyalcohols, polypropylene glycol or mixtures of efhylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

The linker (e.g., a non-nucleotide loop) can be of any length. In some embodiments, the linker has a length equivalent to about 0-16 nucleotides. In some embodiments, the linker has a length equivalent to about 0-8 nucleotides. In some embodiments, the linker has a length equivalent to about 0-4 nucleotides. In some embodiments, the linker has a length equivalent to about 2 nucleotides. Example linker design is also described in WO2011/008730.

A typical Type II Cas9 sgRNA comprises (in 5′ to 3′ direction): a guide sequence, a poly U tract, a first complimentary stretch (the “repeat”), a loop (tetraloop), a second complimentary stretch (the “anti-repeat” being complimentary to the repeat), a stem, and further stem loops and stems and a poly A (often poly U in RNA) tail (terminator). In preferred embodiments, certain aspects of guide architecture are retained, certain aspect of guide architecture cam be modified, for example by addition, subtraction, or substitution of features, whereas certain other aspects of guide architecture are maintained. Preferred locations for engineered sgRNA modifications, including but not limited to insertions, deletions, and substitutions include guide termini and regions of the sgRNA that are exposed when complexed with CRISPR protein and/or target, for example the tetraloop and/or loop2.

In certain embodiments, guides of the invention comprise specific binding sites (e.g. aptamers) for adapter proteins, which may comprise one or more functional domains (e.g. via fusion protein). When such a guides forms a CRISPR complex (i.e. CRISPR enzyme binding to guide and target) the adapter proteins bind and, the functional domain associated with the adapter protein is positioned in a spatial orientation which is advantageous for the attributed function to be effective. For example, if the functional domain is a transcription activator (e.g. VP64 or p65), the transcription activator is placed in a spatial orientation which allows it to affect the transcription of the target. Likewise, a transcription repressor will be advantageously positioned to affect the transcription of the target and a nuclease (e.g. Fok1) will be advantageously positioned to cleave or partially cleave the target.

The skilled person will understand that modifications to the guide which allow for binding of the adapter+functional domain but not proper positioning of the adapter+functional domain (e.g. due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and most preferably at both the tetra loop and stem loop 2.

The repeat:anti repeat duplex will be apparent from the secondary structure of the sgRNA. It may be typically a first complimentary stretch after (in 5′ to 3′ direction) the poly U tract and before the tetraloop; and a second complimentary stretch after (in 5′ to 3′ direction) the tetraloop and before the poly A tract. The first complimentary stretch (the “repeat”) is complimentary to the second complimentary stretch (the “anti-repeat”). As such, they Watson-Crick base pair to form a duplex of dsRNA when folded back on one another. As such, the anti-repeat sequence is the complimentary sequence of the repeat and in terms to A-U or C-G base pairing, but also in terms of the fact that the anti-repeat is in the reverse orientation due to the tetraloop.

In an embodiment of the invention, modification of guide architecture comprises replacing bases in stemloop 2. For example, in some embodiments, “actt” (“acuu” in RNA) and “aagt” (“aagu” in RNA) bases in stemloop2 are replaced with “cgcc” and “gcgg”. In some embodiments, “actt” and “aagt” bases in stemloop2 are replaced with complimentary GC-rich regions of 4 nucleotides. In some embodiments, the complimentary GC-rich regions of 4 nucleotides are “cgcc” and “gcgg” (both in 5′ to 3′ direction). In some embodiments, the complimentary GC-rich regions of 4 nucleotides are “gcgg” and “cgcc” (both in 5′ to 3′ direction). Other combination of C and G in the complimentary GC-rich regions of 4 nucleotides will be apparent including CCCC and GGGG.

In one aspect, the stemloop 2, e.g., “ACTTgtttAAGT” can be replaced by any “XXXXgtttYYYY”, e.g., where XXXX and YYYY represent any complementary sets of nucleotides that together will base pair to each other to create a stem.

In one aspect, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-12 and Y2-12 (wherein X and Y represent any complementary set of nucleotides) may be contemplated. In one aspect, the stem made of the X and Y nucleotides, together with the “gttt,” will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire sgRNA is preserved. In one aspect, the stem can be a form of X:Y basepairing that does not disrupt the secondary structure of the whole sgRNA in that it has a DR:tracr duplex, and 3 stemloops. In one aspect, the “gttt” tetraloop that connects ACTT and AAGT (or any alternative stem made of X:Y basepairs) can be any sequence of the same length (e.g., 4 basepair) or longer that does not interrupt the overall secondary structure of the sgRNA. In one aspect, the stemloop can be something that further lengthens stemloop2, e.g. can be MS2 aptamer. In one aspect, the stemloop3 “GGCACCGagtCGGTGC” can likewise take on a “XXXXXXXagtYYYYYYY” form, e.g., wherein X7 and Y7 represent any complementary sets of nucleotides that together will base pair to each other to create a stem. In one aspect, the stem comprises about 7 bp comprising complementary X and Y sequences, although stems of more or fewer basepairs are also contemplated. In one aspect, the stem made of the X and Y nucleotides, together with the “agt”, will form a complete hairpin in the overall secondary structure. In one aspect, any complementary X:Y basepairing sequence is tolerated, so long as the secondary structure of the entire sgRNA is preserved. In one aspect, the stem can be a form of X:Y basepairing that doesn't disrupt the secondary structure of the whole sgRNA in that it has a DR:tracr duplex, and 3 stemloops. In one aspect, the “agt” sequence of the stemloop 3 can be extended or be replaced by an aptamer, e.g., a MS2 aptamer or sequence that otherwise generally preserves the architecture of stemloop3. In one aspect for alternative Stemloops 2 and/or 3, each X and Y pair can refer to any basepair. In one aspect, non-Watson Crick basepairing is contemplated, where such pairing otherwise generally preserves the architecture of the stemloop at that position.

In one aspect, the DR:tracrRNA duplex can be replaced with the form: gYYYYag(N)NNNNxxxxNNNN(AAN)uuRRRRu (using standard IUPAC nomenclature for nucleotides), wherein (N) and (AAN) represent part of the bulge in the duplex, and “xxxx” represents a linker sequence. NNNN on the direct repeat can be anything so long as it basepairs with the corresponding NNNN portion of the tracrRNA. In one aspect, the DR:tracrRNA duplex can be connected by a linker of any length (xxxx . . . ), any base composition, as long as it doesn't alter the overall structure.

In one aspect, the sgRNA structural requirement is to have a duplex and 3 stemloops. In most aspects, the actual sequence requirement for many of the particular base requirements are lax, in that the architecture of the DR:tracrRNA duplex should be preserved, but the sequence that creates the architecture, i.e., the stems, loops, bulges, etc., may be alterred.

### Aptamers

One guide with a first aptamer/RNA-binding protein pair can be linked or fused to an activator, whilst a second guide with a second aptamer/RNA-binding protein pair can be linked or fused to a repressor. The guides are for different targets (loci), so this allows one gene to be activated and one repressed. For example, the following schematic shows such an approach:

Guide 1—MS2 aptamer-------MS2 RNA-binding protein-------VP64 activator; and
Guide 2—PP7 aptamer-------PP7 RNA-binding protein-------SID4x repressor.

The present invention also relates to orthogonal PP7/MS2 gene targeting. In this example, sgRNA targeting different loci are modified with distinct RNA loops in order to recruit MS2-VP64 or PP7-SID4X, which activate and repress their target loci, respectively. PP7 is the RNA-binding coat protein of the bacteriophage Pseudomonas. Like MS2, it binds a specific RNA sequence and secondary structure. The PP7 RNA-recognition motif is distinct from that of MS2. Consequently, PP7 and MS2 can be multiplexed to mediate distinct effects at different genomic loci simultaneously. For example, an sgRNA targeting locus A can be modified with MS2 loops, recruiting MS2-VP64 activators, while another sgRNA targeting locus B can be modified with PP7 loops, recruiting PP7-SID4X repressor domains. In the same cell, dCas9 can thus mediate orthogonal, locus-specific modifications. This principle can be extended to incorporate other orthogonal RNA-binding proteins such as Q-beta.

An alternative option for orthogonal repression includes incorporating non-coding RNA loops with transactive repressive function into the guide (either at similar positions to the MS2/PP7 loops integrated into the guide or at the 3′ terminus of the guide). For instance, guides were designed with non-coding (but known to be repressive) RNA loops (e.g. using the Alu repressor (in RNA) that interferes with RNA polymerase II in mammalian cells). The Alu RNA sequence was located: in place of the MS2 RNA sequences as used herein (e.g. at tetraloop and/or stem loop 2); and/or at 3′ terminus of the guide. This gives possible combinations of MS2, PP7 or Alu at the tetraloop and/or stemloop 2 positions, as well as, optionally, addition of Alu at the 3′ end of the guide (with or without a linker).

The use of two different aptamers (distinct RNA) allows an activator-adaptor protein fusion and a repressor-adaptor protein fusion to be used, with different guides, to activate expression of one gene, whilst repressing another. They, along with their different guides can be administered together, or substantially together, in a multiplexed approach. A large number of such modified guides can be used all at the same time, for example 10 or 20 or 30 and so forth, whilst only one (or at least a minimal number) of Cas9s to be delivered, as a comparatively small number of Cas9s can be used with a large number modified guides. The adaptor protein may be associated (preferably linked or fused to) one or more activators or one or more repressors. For example, the adaptor protein may be associated with a first activator and a second activator. The first and second activators may be the same, but they are preferably different activators. For example, one might be VP64, whilst the other might be p65, although these are just examples and other transcriptional activators are envisaged. Three or more or even four or more activators (or repressors) may be used, but package size may limit the number being higher than 5 different functional domains. Linkers are preferably used, over a direct fusion to the adaptor protein, where two or more functional domains are associated with the adaptor protein. Suitable linkers might include the GlySer linker.

It is also envisaged that the enzyme-guide complex as a whole may be associated with two or more functional domains. For example, there may be two or more functional domains associated with the enzyme, or there may be two or more functional domains associated with the guide (via one or more adaptor proteins), or there may be one or more functional domains associated with the enzyme and one or more functional domains associated with the guide (via one or more adaptor proteins).

The fusion between the adaptor protein and the activator or repressor may include a linker. For example, GlySer linkers GGGS can be used. They can be used in repeats of 3 ((GGGGS)₃) or 6, 9 or even 12 or more, to provide suitable lengths, as required. Linkers can be used between the RNA-binding protein and the functional domain (activator or repressor), or between the CRISPR Enzyme (Cas9) and the functional domain (activator or repressor). The linkers the user to engineer appropriate amounts of “mechanical flexibility”.

### Dead Guides: Guide RNAs Comprising a Dead Guide Sequence May be Used in the Present Invention

In one aspect, the invention provides guide sequences which are modified in a manner which allows for formation of the CRISPR complex and successful binding to the target, while at the same time, not allowing for successful nuclease activity (i.e. without nuclease activity/without indel activity). For matters of explanation such modified guide sequences are referred to as “dead guides” or “dead guide sequences”. These dead guides or dead guide sequences can be thought of as catalytically inactive or conformationally inactive with regard to nuclease activity. Nuclease activity may be measured using surveyor analysis or deep sequencing as commonly used in the art, preferably surveyor analysis. Similarly, dead guide sequences may not sufficiently engage in productive base pairing with respect to the ability to promote catalytic activity or to distinguish on-target and off-target binding activity. Briefly, the surveyor assay involves purifying and amplifying a CRISPR target site for a gene and forming heteroduplexes with primers amplifying the CRISPR target site. After re-anneal, the products are treated with SURVEYOR nuclease and SURVEYOR enhancer S (Transgenomics) following the manufacturer's recommended protocols, analyzed on gels, and quantified based upon relative band intensities.

Hence, in a related aspect, the invention provides a non-naturally occurring or engineered composition Cas9 CRISPR-Cas system comprising a functional Cas9 as described herein, and guide RNA (gRNA) wherein the gRNA comprises a dead guide sequence whereby the gRNA is capable of hybridizing to a target sequence such that the Cas9 CRISPR-Cas system is directed to a genomic locus of interest in a cell without detectable indel activity resultant from nuclease activity of a non-mutant Cas9 enzyme of the system as detected by a SURVEYOR assay. For shorthand purposes, a gRNA comprising a dead guide sequence whereby the gRNA is capable of hybridizing to a target sequence such that the Cas9 CRISPR-Cas system is directed to a genomic locus of interest in a cell without detectable indel activity resultant from nuclease activity of a non-mutant Cas9 enzyme of the system as detected by a SURVEYOR assay is herein termed a “dead gRNA”. It is to be understood that any of the gRNAs according to the invention as described herein elsewhere may be used as dead gRNAs/gRNAs comprising a dead guide sequence as described herein below. Any of the methods, products, compositions and uses as described herein elsewhere is equally applicable with the dead gRNAs/gRNAs comprising a dead guide sequence as further detailed below. By means of further guidance, the following particular aspects and embodiments are provided.

The ability of a dead guide sequence to direct sequence-specific binding of a CRISPR complex to a target sequence may be assessed by any suitable assay. For example, the components of a CRISPR system sufficient to form a CRISPR complex, including the dead guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the dead guide sequence to be tested and a control guide sequence different from the test dead guide sequence, and comparing binding or rate of cleavage at the target sequence between the test and control guide sequence reactions. Other assays are possible, and will occur to those skilled in the art. A dead guide sequence may be selected to target any target sequence. In some embodiments, the target sequence is a sequence within a genome of a cell.

As explained further herein, several structural parameters allow for a proper framework to arrive at such dead guides. Dead guide sequences are shorter than respective guide sequences which result in active Cas9-specific indel formation. Dead guides are 5%, 10%, 20%, 30%, 40%, 50%, shorter than respective guides directed to the same Cas9 leading to active Cas9-specific indel formation.

As explained below and known in the art, one aspect of gRNA-Cas9 specificity is the direct repeat sequence, which is to be appropriately linked to such guides. In particular, this implies that the direct repeat sequences are designed dependent on the origin of the Cas9. Thus, structural data available for validated dead guide sequences may be used for designing Cas9 specific equivalents. Structural similarity between, e.g., the orthologous nuclease domains RuvC of two or more Cas9 effector proteins may be used to transfer design equivalent dead guides. Thus, the dead guide herein may be appropriately modified in length and sequence to reflect such Cas9 specific equivalents, allowing for formation of the CRISPR complex and successful binding to the target, while at the same time, not allowing for successful nuclease activity.

The use of dead guides in the context herein as well as the state of the art provides a surprising and unexpected platform for network biology and/or systems biology in both in vitro, ex vivo, and in vivo applications, allowing for multiplex gene targeting, and in particular bidirectional multiplex gene targeting. Prior to the use of dead guides, addressing multiple targets, for example for activation, repression and/or silencing of gene activity, has been challenging and in some cases not possible. With the use of dead guides, multiple targets, and thus multiple activities, may be addressed, for example, in the same cell, in the same animal, or in the same patient. Such multiplexing may occur at the same time or staggered for a desired timeframe.

For example, the dead guides now allow for the first time to use gRNA as a means for gene targeting, without the consequence of nuclease activity, while at the same time providing directed means for activation or repression. Guide RNA comprising a dead guide may be modified to further include elements in a manner which allow for activation or repression of gene activity, in particular protein adaptors (e.g. aptamers) as described herein elsewhere allowing for functional placement of gene effectors (e.g. activators or repressors of gene activity). One example is the incorporation of aptamers, as explained herein and in the state of the art. By engineering the gRNA comprising a dead guide to incorporate protein-interacting aptamers (Konermann et al., “Genome-scale transcription activation by an engineered CRISPR-Cas9 complex,” doi:10.1038/nature14136, incorporated herein by reference), one may assemble a synthetic transcription activation complex consisting of multiple distinct effector domains. Such may be modeled after natural transcription activation processes. For example, an aptamer, which selectively binds an effector (e.g. an activator or repressor; dimerized MS2 bacteriophage coat proteins as fusion proteins with an activator or repressor), or a protein which itself binds an effector (e.g. activator or repressor) may be appended to a dead gRNA tetraloop and/or a stem-loop 2. In the case of MS2, the fusion protein MS2-VP64 binds to the tetraloop and/or stem-loop 2 and in turn mediates transcriptional up-regulation, for example for Neurog2. Other transcriptional activators are, for example, VP64. P65, HSF1, and MyoD1. By mere example of this concept, replacement of the MS2 stem-loops with PP7-interacting stem-loops may be used to recruit repressive elements.

Thus, one aspect is a gRNA of the invention which comprises a dead guide, wherein the gRNA further comprises modifications which provide for gene activation or repression, as described herein. The dead gRNA may comprise one or more aptamers. The aptamers may be specific to gene effectors, gene activators or gene repressors. Alternatively, the aptamers may be specific to a protein which in turn is specific to and recruits/binds a specific gene effector, gene activator or gene repressor. If there are multiple sites for activator or repressor recruitment, it is preferred that the sites are specific to either activators or repressors. If there are multiple sites for activator or repressor binding, the sites may be specific to the same activators or same repressors. The sites may also be specific to different activators or different repressors. The gene effectors, gene activators, gene repressors may be present in the form of fusion proteins.

In an embodiment, the dead gRNA as described herein or the Cas9 CRISPR-Cas complex as described herein includes a non-naturally occurring or engineered composition comprising two or more adaptor proteins, wherein each protein is associated with one or more functional domains and wherein the adaptor protein binds to the distinct RNA sequence(s) inserted into the at least one loop of the dead gRNA.

Hence, an aspect provides a non-naturally occurring or engineered composition comprising a guide RNA (gRNA) comprising a dead guide sequence capable of hybridizing to a target sequence in a genomic locus of interest in a cell, wherein the dead guide sequence is as defined herein, a Cas9 comprising at least one or more nuclear localization sequences, wherein the Cas9 optionally comprises at least one mutation wherein at least one loop of the dead gRNA is modified by the insertion of distinct RNA sequence(s) that bind to one or more adaptor proteins, and wherein the adaptor protein is associated with one or more functional domains; or, wherein the dead gRNA is modified to have at least one non-coding functional loop, and wherein the composition comprises two or more adaptor proteins, wherein the each protein is associated with one or more functional domains.

In certain embodiments, the adaptor protein is a fusion protein comprising the functional domain, the fusion protein optionally comprising a linker between the adaptor protein and the functional domain, the linker optionally including a GlySer linker.

In certain embodiments, the at least one loop of the dead gRNA is not modified by the insertion of distinct RNA sequence(s) that bind to the two or more adaptor proteins.

In certain embodiments, the one or more functional domains associated with the adaptor protein is a transcriptional activation domain.

In certain embodiments, the one or more functional domains associated with the adaptor protein is a transcriptional activation domain comprising VP64, p65, MyoD1, HSF1, RTA or SET7/9.

In certain embodiments, the one or more functional domains associated with the adaptor protein is a transcriptional repressor domain.

In certain embodiments, the transcriptional repressor domain is a KRAB domain.

In certain embodiments, the transcriptional repressor domain is a NuE domain, NcoR domain, SID domain or a SID4X domain.

In certain embodiments, at least one of the one or more functional domains associated with the adaptor protein have one or more activities comprising methylase activity, demethylase activity, transcription activation activity, transcription repression activity, transcription release factor activity, histone modification activity, DNA integration activity RNA cleavage activity, DNA cleavage activity or nucleic acid binding activity.

In certain embodiments, the DNA cleavage activity is due to a Fok1 nuclease.

In certain embodiments, the dead gRNA is modified so that, after dead gRNA binds the adaptor protein and further binds to the Cas9 and target, the functional domain is in a spatial orientation allowing for the functional domain to function in its attributed function.

In certain embodiments, the at least one loop of the dead gRNA is tetra loop and/or loop2. In certain embodiments, the tetra loop and loop 2 of the dead gRNA are modified by the insertion of the distinct RNA sequence(s).

In certain embodiments, the insertion of distinct RNA sequence(s) that bind to one or more adaptor proteins is an aptamer sequence. In certain embodiments, the aptamer sequence is two or more aptamer sequences specific to the same adaptor protein. In certain embodiments, the aptamer sequence is two or more aptamer sequences specific to different adaptor protein.

In certain embodiments, the adaptor protein comprises MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, PRR1.

In certain embodiments, the cell is a eukaryotic cell. In certain embodiments, the eukaryotic cell is a mammalian cell, optionally a mouse cell. In certain embodiments, the mammalian cell is a human cell.

In certain embodiments, a first adaptor protein is associated with a p65 domain and a second adaptor protein is associated with a HSF1 domain.

In certain embodiments, the composition comprises a Cas9 CRISPR-Cas complex having at least three functional domains, at least one of which is associated with the Cas9 and at least two of which are associated with dead gRNA.

In certain embodiments, the composition further comprises a second gRNA, wherein the second gRNA is a live gRNA capable of hybridizing to a second target sequence such that a second Cas9 CRISPR-Cas system is directed to a second genomic locus of interest in a cell with detectable indel activity at the second genomic locus resultant from nuclease activity of the Cas9 enzyme of the system.

In certain embodiments, the composition further comprises a plurality of dead gRNAs and/or a plurality of live gRNAs.

One aspect of the invention is to take advantage of the modularity and customizability of the gRNA scaffold to establish a series of gRNA scaffolds with different binding sites (in particular aptamers) for recruiting distinct types of effectors in an orthogonal manner. Again, for matters of example and illustration of the broader concept, replacement of the MS2 stem-loops with PP7-interacting stem-loops may be used to bind/recruit repressive elements, enabling multiplexed bidirectional transcriptional control. Thus, in general, gRNA comprising a dead guide may be employed to provide for multiplex transcriptional control and preferred bidirectional transcriptional control. This transcriptional control is most preferred of genes. For example, one or more gRNA comprising dead guide(s) may be employed in targeting the activation of one or more target genes. At the same time, one or more gRNA comprising dead guide(s) may be employed in targeting the repression of one or more target genes. Such a sequence may be applied in a variety of different combinations, for example the target genes are first repressed and then at an appropriate period other targets are activated, or select genes are repressed at the same time as select genes are activated, followed by further activation and/or repression. As a result, multiple components of one or more biological systems may advantageously be addressed together.

In an aspect, the invention provides nucleic acid molecule(s) encoding dead gRNA or the Cas9 CRISPR-Cas complex or the composition as described herein.

In an aspect, the invention provides a vector system comprising: a nucleic acid molecule encoding dead guide RNA as defined herein. In certain embodiments, the vector system further comprises a nucleic acid molecule(s) encoding Cas9. In certain embodiments, the vector system further comprises a nucleic acid molecule(s) encoding (live) gRNA. In certain embodiments, the nucleic acid molecule or the vector further comprises regulatory element(s) operable in a eukaryotic cell operably linked to the nucleic acid molecule encoding the guide sequence (gRNA) and/or the nucleic acid molecule encoding Cas9 and/or the optional nuclear localization sequence(s).

In another aspect, structural analysis may also be used to study interactions between the dead guide and the active Cas9 nuclease that enable DNA binding, but no DNA cutting. In this way amino acids important for nuclease activity of Cas9 are determined. Modification of such amino acids allows for improved Cas9 enzymes used for gene editing.

A further aspect is combining the use of dead guides as explained herein with other applications of CRISPR, as explained herein as well as known in the art. For example, gRNA comprising dead guide(s) for targeted multiplex gene activation or repression or targeted multiplex bidirectional gene activation/repression may be combined with gRNA comprising guides which maintain nuclease activity, as explained herein. Such gRNA comprising guides which maintain nuclease activity may or may not further include modifications which allow for repression of gene activity (e.g. aptamers). Such gRNA comprising guides which maintain nuclease activity may or may not further include modifications which allow for activation of gene activity (e.g. aptamers). In such a manner, a further means for multiplex gene control is introduced (e.g. multiplex gene targeted activation without nuclease activity/without indel activity may be provided at the same time or in combination with gene targeted repression with nuclease activity).

For example, 1) using one or more gRNA (e.g. 1-50, 1-40, 1-30, 1-20, preferably 1-10, more preferably 1-5) comprising dead guide(s) targeted to one or more genes and further modified with appropriate aptamers for the recruitment of gene activators; 2) may be combined with one or more gRNA (e.g. 1-50, 1-40, 1-30, 1-20, preferably 1-10, more preferably 1-5) comprising dead guide(s) targeted to one or more genes and further modified with appropriate aptamers for the recruitment of gene repressors. 1) and/or 2) may then be combined with 3) one or more gRNA (e.g. 1-50, 1-40, 1-30, 1-20, preferably 1-10, more preferably 1-5) targeted to one or more genes. This combination can then be carried out in turn with 1)+2)+3) with 4) one or more gRNA (e.g. 1-50, 1-40, 1-30, 1-20, preferably 1-10, more preferably 1-5) targeted to one or more genes and further modified with appropriate aptamers for the recruitment of gene activators. This combination can then be carried in turn with 1)+2)+3)+4) with 5) one or more gRNA (e.g. 1-50, 1-40, 1-30, 1-20, preferably 1-10, more preferably 1-5) targeted to one or more genes and further modified with appropriate aptamers for the recruitment of gene repressors. As a result various uses and combinations are included in the invention. For example, combination 1)+2); combination 1)+3); combination 2)+3); combination 1)+2)+3); combination 1)+2)+3)+4); combination 1)+3)+4); combination 2)+3)+4); combination 1)+2)+4); combination 1)+2)+3)+4)+5); combination 1)+3)+4)+5); combination 2)+3)+4)+5); combination 1)+2)+4)+5); combination 1)+2)+3)+5); combination 1)+3)+5); combination 2)+3)+5); combination 1)+2)+5).

In an aspect, the invention provides an algorithm for designing, evaluating, or selecting a dead guide RNA targeting sequence (dead guide sequence) for guiding a Cas9 CRISPR-Cas system to a target gene locus. In particular, it has been determined that dead guide RNA specificity relates to and can be optimized by varying i) GC content and ii) targeting sequence length. In an aspect, the invention provides an algorithm for designing or evaluating a dead guide RNA targeting sequence that minimizes off-target binding or interaction of the dead guide RNA. In an embodiment of the invention, the algorithm for selecting a dead guide RNA targeting sequence for directing a CRISPR system to a gene locus in an organism comprises a) locating one or more CRISPR motifs in the gene locus, analyzing the 20 nt sequence downstream of each CRISPR motif by i) determining the GC content of the sequence; and ii) determining whether there are off-target matches of the 15 downstream nucleotides nearest to the CRISPR motif in the genome of the organism, and c) selecting the 15 nucleotide sequence for use in a dead guide RNA if the GC content of the sequence is 70% or less and no off-target matches are identified. In an embodiment, the sequence is selected for a targeting sequence if the GC content is 60% or less. In certain embodiments, the sequence is selected for a targeting sequence if the GC content is 55% or less, 50% or less, 45% or less, 40% or less, 35% or less or 30% or less. In an embodiment, two or more sequences of the gene locus are analyzed and the sequence having the lowest GC content, or the next lowest GC content, or the next lowest GC content is selected. In an embodiment, the sequence is selected for a targeting sequence if no off-target matches are identified in the genome of the organism. In an embodiment, the targeting sequence is selected if no off-target matches are identified in regulatory sequences of the genome.

In an aspect, the invention provides a method of selecting a dead guide RNA targeting sequence for directing a functionalized CRISPR system to a gene locus in an organism, which comprises: a) locating one or more CRISPR motifs in the gene locus; b) analyzing the 20 nt sequence downstream of each CRISPR motif by: i) determining the GC content of the sequence; and ii) determining whether there are off-target matches of the first 15 nt of the sequence in the genome of the organism; c) selecting the sequence for use in a guide RNA if the GC content of the sequence is 70% or less and no off-target matches are identified. In an embodiment, the sequence is selected if the GC content is 50% or less. In an embodiment, the sequence is selected if the GC content is 40% or less. In an embodiment, the sequence is selected if the GC content is 30% or less. In an embodiment, two or more sequences are analyzed and the sequence having the lowest GC content is selected. In an embodiment, off-target matches are determined in regulatory sequences of the organism. In an embodiment, the gene locus is a regulatory region. An aspect provides a dead guide RNA comprising the targeting sequence selected according to the aforementioned methods.

In an aspect, the invention provides a dead guide RNA for targeting a functionalized CRISPR system to a gene locus in an organism. In an embodiment of the invention, the dead guide RNA comprises a targeting sequence wherein the CG content of the target sequence is 70% or less, and the first 15 nt of the targeting sequence does not match an off-target sequence downstream from a CRISPR motif in the regulatory sequence of another gene locus in the organism. In certain embodiments, the GC content of the targeting sequence 60% or less, 55% or less, 50% or less, 45% or less, 40% or less, 35% or less or 30% or less. In certain embodiments, the GC content of the targeting sequence is from 70% to 60% or from 60% to 50% or from 50% to 40% or from 40% to 30%. In an embodiment, the targeting sequence has the lowest CG content among potential targeting sequences of the locus.

In an embodiment of the invention, the first 15 nt of the dead guide match the target sequence. In another embodiment, first 14 nt of the dead guide match the target sequence. In another embodiment, the first 13 nt of the dead guide match the target sequence. In another embodiment first 12 nt of the dead guide match the target sequence. In another embodiment, first 11 nt of the dead guide match the target sequence. In another embodiment, the first 10 nt of the dead guide match the target sequence. In an embodiment of the invention the first 15 nt of the dead guide does not match an off-target sequence downstream from a CRISPR motif in the regulatory region of another gene locus. In other embodiments, the first 14 nt, or the first 13 nt of the dead guide, or the first 12 nt of the guide, or the first 11 nt of the dead guide, or the first 10 nt of the dead guide, does not match an off-target sequence downstream from a CRISPR motif in the regulatory region of another gene locus. In other embodiments, the first 15 nt, or 14 nt, or 13 nt, or 12 nt, or 11 nt of the dead guide do not match an off-target sequence downstream from a CRISPR motif in the genome.

In certain embodiments, the dead guide RNA includes additional nucleotides at the 3′-end that do not match the target sequence. Thus, a dead guide RNA that includes the first 15 nt, or 14 nt, or 13 nt, or 12 nt, or 11 nt downstream of a CRISPR motif can be extended in length at the 3′ end to 12 nt, 13 nt, 14 nt, 15 nt, 16 nt, 17 nt, 18 nt, 19 nt, 20 nt, or longer.

The invention provides a method for directing a Cas9 CRISPR-Cas system, including but not limited to a dead Cas9 (dCas9) or functionalized Cas9 system (which may comprise a functionalized Cas9 or functionalized guide) to a gene locus. In an aspect, the invention provides a method for selecting a dead guide RNA targeting sequence and directing a functionalized CRISPR system to a gene locus in an organism. In an aspect, the invention provides a method for selecting a dead guide RNA targeting sequence and effecting gene regulation of a target gene locus by a functionalized Cas9 CRISPR-Cas system. In certain embodiments, the method is used to effect target gene regulation while minimizing off-target effects. In an aspect, the invention provides a method for selecting two or more dead guide RNA targeting sequences and effecting gene regulation of two or more target gene loci by a functionalized Cas9 CRISPR-Cas system. In certain embodiments, the method is used to effect regulation of two or more target gene loci while minimizing off-target effects.

In an aspect, the invention provides a method of selecting a dead guide RNA targeting sequence for directing a functionalized Cas9 to a gene locus in an organism, which comprises: a) locating one or more CRISPR motifs in the gene locus; b) analyzing the sequence downstream of each CRISPR motif by: i) selecting 10 to 15 nt adjacent to the CRISPR motif, ii) determining the GC content of the sequence; and c) selecting the 10 to 15 nt sequence as a targeting sequence for use in a guide RNA if the GC content of the sequence is 40% or more. In an embodiment, the sequence is selected if the GC content is 50% or more. In an embodiment, the sequence is selected if the GC content is 60% or more. In an embodiment, the sequence is selected if the GC content is 70% or more. In an embodiment, two or more sequences are analyzed and the sequence having the highest GC content is selected. In an embodiment, the method further comprises adding nucleotides to the 3′ end of the selected sequence which do not match the sequence downstream of the CRISPR motif. An aspect provides a dead guide RNA comprising the targeting sequence selected according to the aforementioned methods.

In an aspect, the invention provides a dead guide RNA for directing a functionalized CRISPR system to a gene locus in an organism wherein the targeting sequence of the dead guide RNA consists of 10 to 15 nucleotides adjacent to the CRISPR motif of the gene locus, wherein the CG content of the target sequence is 50% or more. In certain embodiments, the dead guide RNA further comprises nucleotides added to the 3′ end of the targeting sequence which do not match the sequence downstream of the CRISPR motif of the gene locus.

In an aspect, the invention provides for a single effector to be directed to one or more, or two or more gene loci. In certain embodiments, the effector is associated with a Cas9, and one or more, or two or more selected dead guide RNAs are used to direct the Cas9-associated effector to one or more, or two or more selected target gene loci. In certain embodiments, the effector is associated with one or more, or two or more selected dead guide RNAs, each selected dead guide RNA, when complexed with a Cas9 enzyme, causing its associated effector to localize to the dead guide RNA target. One non-limiting example of such CRISPR systems modulates activity of one or more, or two or more gene loci subject to regulation by the same transcription factor.

In an aspect, the invention provides for two or more effectors to be directed to one or more gene loci. In certain embodiments, two or more dead guide RNAs are employed, each of the two or more effectors being associated with a selected dead guide RNA, with each of the two or more effectors being localized to the selected target of its dead guide RNA. One non-limiting example of such CRISPR systems modulates activity of one or more, or two or more gene loci subject to regulation by different transcription factors. Thus, in one non-limiting embodiment, two or more transcription factors are localized to different regulatory sequences of a single gene. In another non-limiting embodiment, two or more transcription factors are localized to different regulatory sequences of different genes. In certain embodiments, one transcription factor is an activator. In certain embodiments, one transcription factor is an inhibitor. In certain embodiments, one transcription factor is an activator and another transcription factor is an inhibitor. In certain embodiments, gene loci expressing different components of the same regulatory pathway are regulated. In certain embodiments, gene loci expressing components of different regulatory pathways are regulated.

In an aspect, the invention also provides a method and algorithm for designing and selecting dead guide RNAs that are specific for target DNA cleavage or target binding and gene regulation mediated by an active Cas9 CRISPR-Cas system. In certain embodiments, the Cas9 CRISPR-Cas system provides orthogonal gene control using an active Cas9 which cleaves target DNA at one gene locus while at the same time binds to and promotes regulation of another gene locus.

In an aspect, the invention provides an method of selecting a dead guide RNA targeting sequence for directing a functionalized Cas9 to a gene locus in an organism, without cleavage, which comprises a) locating one or more CRISPR motifs in the gene locus; b) analyzing the sequence downstream of each CRISPR motif by i) selecting 10 to 15 nt adjacent to the CRISPR motif, ii) determining the GC content of the sequence, and c) selecting the 10 to 15 nt sequence as a targeting sequence for use in a dead guide RNA if the GC content of the sequence is 30% more, 40% or more. In certain embodiments, the GC content of the targeting sequence is 35% or more, 40% or more, 45% or more, 50% or more, 55% or more, 60% or more, 65% or more, or 70% or more. In certain embodiments, the GC content of the targeting sequence is from 30% to 40% or from 40% to 50% or from 50% to 60% or from 60% to 70%. In an embodiment of the invention, two or more sequences in a gene locus are analyzed and the sequence having the highest GC content is selected.

In an embodiment of the invention, the portion of the targeting sequence in which GC content is evaluated is 10 to 15 contiguous nucleotides of the 15 target nucleotides nearest to the PAM. In an embodiment of the invention, the portion of the guide in which GC content is considered is the 10 to 11 nucleotides or 11 to 12 nucleotides or 12 to 13 nucleotides or 13, or 14, or 15 contiguous nucleotides of the 15 nucleotides nearest to the PAM.

In an aspect, the invention further provides an algorithm for identifying dead guide RNAs which promote CRISPR system gene locus cleavage while avoiding functional activation or inhibition. It is observed that increased GC content in dead guide RNAs of 16 to 20 nucleotides coincides with increased DNA cleavage and reduced functional activation.

It is also demonstrated herein that efficiency of functionalized Cas9 can be increased by addition of nucleotides to the 3′ end of a guide RNA which do not match a target sequence downstream of the CRISPR motif. For example, of dead guide RNA 11 to 15 nt in length, shorter guides may be less likely to promote target cleavage, but are also less efficient at promoting CRISPR system binding and functional control. In certain embodiments, addition of nucleotides that don't match the target sequence to the 3′ end of the dead guide RNA increase activation efficiency while not increasing undesired target cleavage. In an aspect, the invention also provides a method and algorithm for identifying improved dead guide RNAs that effectively promote CRISPRP system function in DNA binding and gene regulation while not promoting DNA cleavage. Thus, in certain embodiments, the invention provides a dead guide RNA that includes the first 15 nt, or 14 nt, or 13 nt, or 12 nt, or 11 nt downstream of a CRISPR motif and is extended in length at the 3′ end by nucleotides that mismatch the target to 12 nt, 13 nt, 14 nt, 15 nt, 16 nt, 17 nt, 18 nt, 19 nt, 20 nt, or longer.

In an aspect, the invention provides a method for effecting selective orthogonal gene control. As will be appreciated from the disclosure herein, dead guide selection according to the invention, taking into account guide length and GC content, provides effective and selective transcription control by a functional Cas9 CRISPR-Cas system, for example to regulate transcription of a gene locus by activation or inhibition and minimize off-target effects. Accordingly, by providing effective regulation of individual target loci, the invention also provides effective orthogonal regulation of two or more target loci.

In certain embodiments, orthogonal gene control is by activation or inhibition of two or more target loci. In certain embodiments, orthogonal gene control is by activation or inhibition of one or more target locus and cleavage of one or more target locus.

In one aspect, the invention provides a cell comprising a non-naturally occurring Cas9 CRISPR-Cas system comprising one or more dead guide RNAs disclosed or made according to a method or algorithm described herein wherein the expression of one or more gene products has been altered. In an embodiment of the invention, the expression in the cell of two or more gene products has been altered. The invention also provides a cell line from such a cell.

In one aspect, the invention provides a multicellular organism comprising one or more cells comprising a non-naturally occurring Cas9 CRISPR-Cas system comprising one or more dead guide RNAs disclosed or made according to a method or algorithm described herein. In one aspect, the invention provides a product from a cell, cell line, or multicellular organism comprising a non-naturally occurring Cas9 CRISPR-Cas system comprising one or more dead guide RNAs disclosed or made according to a method or algorithm described herein.

A further aspect of this invention is the use of gRNA comprising dead guide(s) as described herein, optionally in combination with gRNA comprising guide(s) as described herein or in the state of the art, in combination with systems e.g. cells, transgenic animals, transgenic mice, inducible transgenic animals, inducible transgenic mice) which are engineered for either overexpression of Cas9 or preferably knock in Cas9. As a result a single system (e.g. transgenic animal, cell) can serve as a basis for multiplex gene modifications in systems/network biology. On account of the dead guides, this is now possible in both in vitro, ex vivo, and in vivo.

For example, once the Cas9 is provided for, one or more dead gRNAs may be provided to direct multiplex gene regulation, and preferably multiplex bidirectional gene regulation. The one or more dead gRNAs may be provided in a spatially and temporally appropriate manner if necessary or desired (for example tissue specific induction of Cas9 expression). On account that the transgenic/inducible Cas9 is provided for (e.g. expressed) in the cell, tissue, animal of interest, both gRNAs comprising dead guides or gRNAs comprising guides are equally effective. In the same manner, a further aspect of this invention is the use of gRNA comprising dead guide(s) as described herein, optionally in combination with gRNA comprising guide(s) as described herein or in the state of the art, in combination with systems (e.g. cells, transgenic animals, transgenic mice, inducible transgenic animals, inducible transgenic mice) which are engineered for knockout Cas9 CRISPR-Cas.

As a result, the combination of dead guides as described herein with CRISPR applications described herein and CRISPR applications known in the art results in a highly efficient and accurate means for multiplex screening of systems (e.g. network biology). Such screening allows, for example, identification of specific combinations of gene activities for identifying genes responsible for diseases (e.g. on/off combinations), in particular gene related diseases. A preferred application of such screening is cancer. In the same manner, screening for treatment for such diseases is included in the invention. Cells or animals may be exposed to aberrant conditions resulting in disease or disease like effects. Candidate compositions may be provided and screened for an effect in the desired multiplex environment. For example a patient's cancer cells may be screened for which gene combinations will cause them to die, and then use this information to establish appropriate therapies.

In one aspect, the invention provides a kit comprising one or more of the components described herein. The kit may include dead guides as described herein with or without guides as described herein.

The structural information provided herein allows for interrogation of dead gRNA interaction with the target DNA and the Cas9 permitting engineering or alteration of dead gRNA structure to optimize functionality of the entire Cas9 CRISPR-Cas system. For example, loops of the dead gRNA may be extended, without colliding with the Cas9 protein by the insertion of adaptor proteins that can bind to RNA. These adaptor proteins can further recruit effector proteins or fusions which comprise one or more functional domains.

In some preferred embodiments, the functional domain is a transcriptional activation domain, preferably VP64. In some embodiments, the functional domain is a transcription repression domain, preferably KRAB. In some embodiments, the transcription repression domain is SID, or concatemers of SID (e.g. SID4X). In some embodiments, the functional domain is an epigenetic modifying domain, such that an epigenetic modifying enzyme is provided. In some embodiments, the functional domain is an activation domain, which may be the P65 activation domain.

An aspect of the invention is that the above elements are comprised in a single composition or comprised in individual compositions. These compositions may advantageously be applied to a host to elicit a functional effect on the genomic level.

In general, the dead gRNA are modified in a manner that provides specific binding sites (e.g. aptamers) for adapter proteins comprising one or more functional domains (e.g. via fusion protein) to bind to. The modified dead gRNA are modified such that once the dead gRNA forms a CRISPR complex (i.e. Cas9 binding to dead gRNA and target) the adapter proteins bind and, the functional domain on the adapter protein is positioned in a spatial orientation which is advantageous for the attributed function to be effective. For example, if the functional domain is a transcription activator (e.g. VP64 or p65), the transcription activator is placed in a spatial orientation which allows it to affect the transcription of the target. Likewise, a transcription repressor will be advantageously positioned to affect the transcription of the target and a nuclease (e.g. Fok1) will be advantageously positioned to cleave or partially cleave the target.

The skilled person will understand that modifications to the dead gRNA which allow for binding of the adapter+functional domain but not proper positioning of the adapter+functional domain (e.g. due to steric hindrance within the three dimensional structure of the CRISPR complex) are modifications which are not intended. The one or more modified dead gRNA may be modified at the tetra loop, the stem loop 1, stem loop 2, or stem loop 3, as described herein, preferably at either the tetra loop or stem loop 2, and most preferably at both the tetra loop and stem loop 2.

As explained herein the functional domains may be, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g. light inducible). In some cases it is advantageous that additionally at least one NLS is provided. In some instances, it is advantageous to position the NLS at the N terminus. When more than one functional domain is included, the functional domains may be the same or different.

The dead gRNA may be designed to include multiple binding recognition sites (e.g. aptamers) specific to the same or different adapter protein. The dead gRNA may be designed to bind to the promoter region −1000-+1 nucleic acids upstream of the transcription start site (i.e. TSS), preferably −200 nucleic acids. This positioning improves functional domains which affect gene activation (e.g. transcription activators) or gene inhibition (e.g. transcription repressors). The modified dead gRNA may be one or more modified dead gRNAs targeted to one or more target loci (e.g. at least 1 gRNA, at least 2 gRNA, at least 5 gRNA, at least 10 gRNA, at least 20 gRNA, at least 30 gRNA, at least 50 gRNA) comprised in a composition.

The adaptor protein may be any number of proteins that binds to an aptamer or recognition site introduced into the modified dead gRNA and which allows proper positioning of one or more functional domains, once the dead gRNA has been incorporated into the CRISPR complex, to affect the target with the attributed function. As explained in detail in this application such may be coat proteins, preferably bacteriophage coat proteins. The functional domains associated with such adaptor proteins (e.g. in the form of fusion protein) may include, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g. light inducible). Preferred domains are Fok1, VP64, P65, HSF1, MyoD1. In the event that the functional domain is a transcription activator or transcription repressor it is advantageous that additionally at least an NLS is provided and preferably at the N terminus. When more than one functional domain is included, the functional domains may be the same or different. The adaptor protein may utilize known linkers to attach such functional domains.

Thus, the modified dead gRNA, the (inactivated) Cas9 (with or without functional domains), and the binding protein with one or more functional domains, may each individually be comprised in a composition and administered to a host individually or collectively. Alternatively, these components may be provided in a single composition for administration to a host. Administration to a host may be performed via viral vectors known to the skilled person or described herein for delivery to a host (e.g. lentiviral vector, adenoviral vector, AAV vector). As explained herein, use of different selection markers (e.g. for lentiviral gRNA selection) and concentration of gRNA (e.g. dependent on whether multiple gRNAs are used) may be advantageous for eliciting an improved effect.

On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. The compositions may be applied in a wide variety of methods for screening in libraries in cells and functional modeling in vivo (e.g. gene activation of lincRNA and identification of function; gain-of-function modeling; loss-of-function modeling; the use the compositions of the invention to establish cell lines and transgenic animals for optimization and screening purposes).

The current invention comprehends the use of the compositions of the current invention to establish and utilize conditional or inducible CRISPR transgenic cell/animals, which are not believed prior to the present invention or application. For example, the target cell comprises Cas9 conditionally or inducibly (e.g. in the form of Cre dependent constructs) and/or the adapter protein conditionally or inducibly and, on expression of a vector introduced into the target cell, the vector expresses that which induces or gives rise to the condition of Cas9 expression and/or adaptor expression in the target cell. By applying the teaching and compositions of the current invention with the known method of creating a CRISPR complex, inducible genomic events affected by functional domains are also an aspect of the current invention. One example of this is the creation of a CRISPR knock-in/conditional transgenic animal (e.g. mouse comprising e.g. a Lox-Stop-polyA-Lox(LSL) cassette) and subsequent delivery of one or more compositions providing one or more modified dead gRNA (e.g. −200 nucleotides to TSS of a target gene of interest for gene activation purposes) as described herein (e.g. modified dead gRNA with one or more aptamers recognized by coat proteins, e.g. MS2), one or more adapter proteins as described herein (MS2 binding protein linked to one or more VP64) and means for inducing the conditional animal (e.g. Cre recombinase for rendering Cas9 expression inducible). Alternatively, the adaptor protein may be provided as a conditional or inducible element with a conditional or inducible Cas9 to provide an effective model for screening purposes, which advantageously only requires minimal design and administration of specific dead gRNAs for a broad number of applications.

In another aspect the dead guides are further modified to improve specificity. Protected dead guides may be synthesized, whereby secondary structure is introduced into the 3′ end of the dead guide to improve its specificity. A protected guide RNA (pgRNA) comprises a guide sequence capable of hybridizing to a target sequence in a genomic locus of interest in a cell and a protector strand, wherein the protector strand is optionally complementary to the guide sequence and wherein the guide sequence may in part be hybridizable to the protector strand. The pgRNA optionally includes an extension sequence. The thermodynamics of the pgRNA-target DNA hybridization is determined by the number of bases complementary between the guide RNA and target DNA. By employing ‘thermodynamic protection’, specificity of dead gRNA can be improved by adding a protector sequence. For example, one method adds a complementary protector strand of varying lengths to the 3′ end of the guide sequence within the dead gRNA. As a result, the protector strand is bound to at least a portion of the dead gRNA and provides for a protected gRNA (pgRNA). In turn, the dead gRNA references herein may be easily protected using the described embodiments, resulting in pgRNA. The protector strand can be either a separate RNA transcript or strand or a chimeric version joined to the 3′ end of the dead gRNA guide sequence.

### Tandem Guides and Uses in a Multiplex (Tandem) Targeting Approach

The inventors have shown that CRISPR enzymes as defined herein can employ more than one RNA guide without losing activity. This enables the use of the CRISPR enzymes, systems or complexes as defined herein for targeting multiple DNA targets, genes or gene loci, with a single enzyme, system or complex as defined herein. The guide RNAs may be tandemly arranged, optionally separated by a nucleotide sequence such as a direct repeat as defined herein. The position of the different guide RNAs is the tandem does not influence the activity. It is noted that the terms “CRISPR-Cas system”, “CRISP-Cas complex” “CRISPR complex” and “CRISPR system” are used interchangeably. Also the terms “CRISPR enzyme”, “Cas enzyme”, or “CRISPR-Cas enzyme”, can be used interchangeably. In preferred embodiments, said CRISPR enzyme, CRISP-Cas enzyme or Cas enzyme is Cas9, or any one of the modified or mutated variants thereof described herein elsewhere.

In one aspect, the invention provides a non-naturally occurring or engineered CRISPR enzyme, preferably a class 2 CRISPR enzyme, preferably a Type V or VI CRISPR enzyme as described herein, such as without limitation Cas9 as described herein elsewhere, used for tandem or multiplex targeting. It is to be understood that any of the CRISPR (or CRISPR-Cas or Cas) enzymes, complexes, or systems according to the invention as described herein elsewhere may be used in such an approach. Any of the methods, products, compositions and uses as described herein elsewhere are equally applicable with the multiplex or tandem targeting approach further detailed below. By means of further guidance, the following particular aspects and embodiments are provided.

In one aspect, the invention provides for the use of a Cas9 enzyme, complex or system as defined herein for targeting multiple gene loci. In one embodiment, this can be established by using multiple (tandem or multiplex) guide RNA (gRNA) sequences.

In one aspect, the invention provides methods for using one or more elements of a Cas9 enzyme, complex or system as defined herein for tandem or multiplex targeting, wherein said CRISPR system comprises multiple guide RNA sequences. Preferably, said gRNA sequences are separated by a nucleotide sequence, such as a direct repeat as defined herein elsewhere.

The Cas9 enzyme, system or complex as defined herein provides an effective means for modifying multiple target polynucleotides. The Cas9 enzyme, system or complex as defined herein has a wide variety of utility including modifying (e.g., deleting, inserting, translocating, inactivating, activating) one or more target polynucleotides in a multiplicity of cell types. As such the Cas9 enzyme, system or complex as defined herein of the invention has a broad spectrum of applications in, e.g., gene therapy, drug screening, disease diagnosis, and prognosis, including targeting multiple gene loci within a single CRISPR system.

In one aspect, the invention provides a Cas9 enzyme, system or complex as defined herein, i.e. a Cas9 CRISPR-Cas complex having a Cas9 protein having at least one destabilization domain associated therewith, and multiple guide RNAs that target multiple nucleic acid molecules such as DNA molecules, whereby each of said multiple guide RNAs specifically targets its corresponding nucleic acid molecule, e.g., DNA molecule. Each nucleic acid molecule target, e.g., DNA molecule can encode a gene product or encompass a gene locus. Using multiple guide RNAs hence enables the targeting of multiple gene loci or multiple genes. In some embodiments the Cas9 enzyme may cleave the DNA molecule encoding the gene product. In some embodiments expression of the gene product is altered. The Cas9 protein and the guide RNAs do not naturally occur together. The invention comprehends the guide RNAs comprising tandemly arranged guide sequences. The invention further comprehends coding sequences for the Cas9 protein being codon optimized for expression in a eukaryotic cell. In a preferred embodiment the eukaryotic cell is a mammalian cell, a plant cell or a yeast cell and in a more preferred embodiment the mammalian cell is a human cell. Expression of the gene product may be decreased. The Cas9 enzyme may form part of a CRISPR system or complex, which further comprises tandemly arranged guide RNAs (gRNAs) comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In some embodiments, the functional Cas9 CRISPR system or complex binds to the multiple target sequences. In some embodiments, the functional CRISPR system or complex may edit the multiple target sequences, e.g., the target sequences may comprise a genomic locus, and in some embodiments there may be an alteration of gene expression. In some embodiments, the functional CRISPR system or complex may comprise further functional domains. In some embodiments, the invention provides a method for altering or modifying expression of multiple gene products. The method may comprise introducing into a cell containing said target nucleic acids, e.g., DNA molecules, or containing and expressing target nucleic acid, e.g., DNA molecules; for instance, the target nucleic acids may encode gene products or provide for expression of gene products (e.g., regulatory sequences).

In preferred embodiments the CRISPR enzyme used for multiplex targeting is Cas9, or the CRISPR system or complex comprises Cas9. In some embodiments, the CRISPR enzyme used for multiplex targeting is AsCas9, or the CRISPR system or complex used for multiplex targeting comprises an AsCas9. In some embodiments, the CRISPR enzyme is an LbCas9, or the CRISPR system or complex comprises LbCas9. In some embodiments, the Cas9 enzyme used for multiplex targeting cleaves both strands of DNA to produce a double strand break (DSB). In some embodiments, the CRISPR enzyme used for multiplex targeting is a nickase. In some embodiments, the Cas9 enzyme used for multiplex targeting is a dual nickase. In some embodiments, the Cas9 enzyme used for multiplex targeting is a Cas9 enzyme such as a DD Cas9 enzyme as defined herein elsewhere.

In some general embodiments, the Cas9 enzyme used for multiplex targeting is associated with one or more functional domains. In some more specific embodiments, the CRISPR enzyme used for multiplex targeting is a deadCas9 as defined herein elsewhere.

In an aspect, the present invention provides a means for delivering the Cas9 enzyme, system or complex for use in multiple targeting as defined herein or the polynucleotides defined herein. Non-limiting examples of such delivery means are e.g. particle(s) delivering component(s) of the complex, vector(s) comprising the polynucleotide(s) discussed herein (e.g., encoding the CRISPR enzyme, providing the nucleotides encoding the CRISPR complex). In some embodiments, the vector may be a plasmid or a viral vector such as AAV, or lentivirus. Transient transfection with plasmids, e.g., into HEK cells may be advantageous, especially given the size limitations of AAV and that while Cas9 fits into AAV, one may reach an upper limit with additional guide RNAs.

Also provided is a model that constitutively expresses the Cas9 enzyme, complex or system as used herein for use in multiplex targeting. The organism may be transgenic and may have been transfected with the present vectors or may be the offspring of an organism so transfected. In a further aspect, the present invention provides compositions comprising the CRISPR enzyme, system and complex as defined herein or the polynucleotides or vectors described herein. Also provides are Cas9 CRISPR systems or complexes comprising multiple guide RNAs, preferably in a tandemly arranged format. Said different guide RNAs may be separated by nucleotide sequences such as direct repeats.

Also provided is a method of treating a subject, e.g., a subject in need thereof, comprising inducing gene editing by transforming the subject with the polynucleotide encoding the Cas9 CRISPR system or complex or any of polynucleotides or vectors described herein and administering them to the subject. A suitable repair template may also be provided, for example delivered by a vector comprising said repair template. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises the Cas9 enzyme, complex or system comprising multiple guide RNAs, preferably tandemly arranged. Where any treatment is occurring ex vivo, for example in a cell culture, then it will be appreciated that the term ‘subject’ may be replaced by the phrase “cell or cell culture.”

Compositions comprising Cas9 enzyme, complex or system comprising multiple guide RNAs, preferably tandemly arranged, or the polynucleotide or vector encoding or comprising said Cas9 enzyme, complex or system comprising multiple guide RNAs, preferably tandemly arranged, for use in the methods of treatment as defined herein elsewhere are also provided. A kit of parts may be provided including such compositions. Use of said composition in the manufacture of a medicament for such methods of treatment are also provided. Use of a Cas9 CRISPR system in screening is also provided by the present invention, e.g., gain of function screens. Cells which are artificially forced to overexpress a gene are be able to down regulate the gene over time (re-establishing equilibrium) e.g. by negative feedback loops. By the time the screen starts the unregulated gene might be reduced again. Using an inducible Cas9 activator allows one to induce transcription right before the screen and therefore minimizes the chance of false negative hits. Accordingly, by use of the instant invention in screening, e.g., gain of function screens, the chance of false negative results may be minimized.

In one aspect, the invention provides an engineered, non-naturally occurring CRISPR system comprising a Cas9 protein and multiple guide RNAs that each specifically target a DNA molecule encoding a gene product in a cell, whereby the multiple guide RNAs each target their specific DNA molecule encoding the gene product and the Cas9 protein cleaves the target DNA molecule encoding the gene product, whereby expression of the gene product is altered; and, wherein the CRISPR protein and the guide RNAs do not naturally occur together. The invention comprehends the multiple guide RNAs comprising multiple guide sequences, preferably separated by a nucleotide sequence such as a direct repeat and optionally fused to a tracr sequence. In an embodiment of the invention the CRISPR protein is a type V or VI CRISPR-Cas protein and in a more preferred embodiment the CRISPR protein is a Cas9 protein. The invention further comprehends a Cas9 protein being codon optimized for expression in a eukaryotic cell. In a preferred embodiment the eukaryotic cell is a mammalian cell and in a more preferred embodiment the mammalian cell is a human cell. In a further embodiment of the invention, the expression of the gene product is decreased.

In another aspect, the invention provides an engineered, non-naturally occurring vector system comprising one or more vectors comprising a first regulatory element operably linked to the multiple Cas9 CRISPR system guide RNAs that each specifically target a DNA molecule encoding a gene product and a second regulatory element operably linked coding for a CRISPR protein. Both regulatory elements may be located on the same vector or on different vectors of the system. The multiple guide RNAs target the multiple DNA molecules encoding the multiple gene products in a cell and the CRISPR protein may cleave the multiple DNA molecules encoding the gene products (it may cleave one or both strands or have substantially no nuclease activity), whereby expression of the multiple gene products is altered; and, wherein the CRISPR protein and the multiple guide RNAs do not naturally occur together. In a preferred embodiment the CRISPR protein is Cas9 protein, optionally codon optimized for expression in a eukaryotic cell. In a preferred embodiment the eukaryotic cell is a mammalian cell, a plant cell or a yeast cell and in a more preferred embodiment the mammalian cell is a human cell. In a further embodiment of the invention, the expression of each of the multiple gene products is altered, preferably decreased.

In one aspect, the invention provides a vector system comprising one or more vectors. In some embodiments, the system comprises: (a) a first regulatory element operably linked to a direct repeat sequence and one or more insertion sites for inserting one or more guide sequences up- or downstream (whichever applicable) of the direct repeat sequence, wherein when expressed, the one or more guide sequence(s) direct(s) sequence-specific binding of the CRISPR complex to the one or more target sequence(s) in a eukaryotic cell, wherein the CRISPR complex comprises a Cas9 enzyme complexed with the one or more guide sequence(s) that is hybridized to the one or more target sequence(s); and (b) a second regulatory element operably linked to an enzyme-coding sequence encoding said Cas9 enzyme, preferably comprising at least one nuclear localization sequence and/or at least one NES; wherein components (a) and (b) are located on the same or different vectors of the system. Where applicable, a tracr sequence may also be provided. In some embodiments, component (a) further comprises two or more guide sequences operably linked to the first regulatory element, wherein when expressed, each of the two or more guide sequences direct sequence specific binding of a Cas9 CRISPR complex to a different target sequence in a eukaryotic cell. In some embodiments, the CRISPR complex comprises one or more nuclear localization sequences and/or one or more NES of sufficient strength to drive accumulation of said Cas9 CRISPR complex in a detectable amount in or out of the nucleus of a eukaryotic cell. In some embodiments, the first regulatory element is a polymerase III promoter. In some embodiments, the second regulatory element is a polymerase II promoter. In some embodiments, each of the guide sequences is at least 16, 17, 18, 19, 20, 25 nucleotides, or between 16-30, or between 16-25, or between 16-20 nucleotides in length.

Recombinant expression vectors can comprise the polynucleotides encoding the Cas9 enzyme, system or complex for use in multiple targeting as defined herein in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

In some embodiments, a host cell is transiently or non-transiently transfected with one or more vectors comprising the polynucleotides encoding the Cas9 enzyme, system or complex for use in multiple targeting as defined herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art and exemplified herein elsewhere. Cell lines are available from a variety of sources known to those with skill in the art (see, e.g., the American Type Culture Collection (ATCC) (Manassas, Va.)). In some embodiments, a cell transfected with one or more vectors comprising the polynucleotides encoding the Cas9 enzyme, system or complex for use in multiple targeting as defined herein is used to establish a new cell line comprising one or more vector-derived sequences. In some embodiments, a cell transiently transfected with the components of a Cas9 CRISPR system or complex for use in multiple targeting as described herein (such as by transient transfection of one or more vectors, or transfection with RNA), and modified through the activity of a Cas9 CRISPR system or complex, is used to establish a new cell line comprising cells containing the modification but lacking any other exogenous sequence. In some embodiments, cells transiently or non-transiently transfected with one or more vectors comprising the polynucleotides encoding the Cas9 enzyme, system or complex for use in multiple targeting as defined herein, or cell lines derived from such cells are used in assessing one or more test compounds.

The term “regulatory element” is as defined herein elsewhere.

Advantageous vectors include lentiviruses and adeno-associated viruses, and types of such vectors can also be selected for targeting particular types of cells.

In one aspect, the invention provides a eukaryotic host cell comprising (a) a first regulatory element operably linked to a direct repeat sequence and one or more insertion sites for inserting one or more guide RNA sequences up- or downstream (whichever applicable) of the direct repeat sequence, wherein when expressed, the guide sequence(s) direct(s) sequence-specific binding of the Cas9 CRISPR complex to the respective target sequence(s) in a eukaryotic cell, wherein the Cas9 CRISPR complex comprises a Cas9 enzyme complexed with the one or more guide sequence(s) that is hybridized to the respective target sequence(s); and/or (b) a second regulatory element operably linked to an enzyme-coding sequence encoding said Cas9 enzyme comprising preferably at least one nuclear localization sequence and/or NES. In some embodiments, the host cell comprises components (a) and (b). Where applicable, a tracr sequence may also be provided. In some embodiments, component (a), component (b), or components (a) and (b) are stably integrated into a genome of the host eukaryotic cell. In some embodiments, component (a) further comprises two or more guide sequences operably linked to the first regulatory element, and optionally separated by a direct repeat, wherein when expressed, each of the two or more guide sequences direct sequence specific binding of a Cas9 CRISPR complex to a different target sequence in a eukaryotic cell. In some embodiments, the Cas9 enzyme comprises one or more nuclear localization sequences and/or nuclear export sequences or NES of sufficient strength to drive accumulation of said CRISPR enzyme in a detectable amount in and/or out of the nucleus of a eukaryotic cell.

In some embodiments, the Cas9 enzyme is a type V or VI CRISPR system enzyme. In some embodiments, the Cas9 enzyme is a Cas9 enzyme. In some embodiments, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9, and may include further alterations or mutations of the Cas9 as defined herein elsewhere, and can be a chimeric Cas9. In some embodiments, the Cas9 enzyme is codon-optimized for expression in a eukaryotic cell. In some embodiments, the CRISPR enzyme directs cleavage of one or two strands at the location of the target sequence. In some embodiments, the first regulatory element is a polymerase III promoter. In some embodiments, the second regulatory element is a polymerase II promoter. In some embodiments, the one or more guide sequence(s) is (are each) at least 16, 17, 18, 19, 20, 25 nucleotides, or between 16-30, or between 16-25, or between 16-20 nucleotides in length. When multiple guide RNAs are used, they are preferably separated by a direct repeat sequence. In an aspect, the invention provides a non-human eukaryotic organism; preferably a multicellular eukaryotic organism, comprising a eukaryotic host cell according to any of the described embodiments. In other aspects, the invention provides a eukaryotic organism; preferably a multicellular eukaryotic organism, comprising a eukaryotic host cell according to any of the described embodiments. The organism in some embodiments of these aspects may be an animal; for example a mammal. Also, the organism may be an arthropod such as an insect. The organism also may be a plant. Further, the organism may be a fungus.

In one aspect, the invention provides a kit comprising one or more of the components described herein. In some embodiments, the kit comprises a vector system and instructions for using the kit. In some embodiments, the vector system comprises (a) a first regulatory element operably linked to a direct repeat sequence and one or more insertion sites for inserting one or more guide sequences up- or downstream (whichever applicable) of the direct repeat sequence, wherein when expressed, the guide sequence directs sequence-specific binding of a Cas9 CRISPR complex to a target sequence in a eukaryotic cell, wherein the Cas9 CRISPR complex comprises a Cas9 enzyme complexed with the guide sequence that is hybridized to the target sequence; and/or (b) a second regulatory element operably linked to an enzyme-coding sequence encoding said Cas9 enzyme comprising a nuclear localization sequence. Where applicable, a tracr sequence may also be provided. In some embodiments, the kit comprises components (a) and (b) located on the same or different vectors of the system. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the Cas9 enzyme comprises one or more nuclear localization sequences of sufficient strength to drive accumulation of said CRISPR enzyme in a detectable amount in the nucleus of a eukaryotic cell. In some embodiments, the CRISPR enzyme is a type V or VI CRISPR system enzyme. In some embodiments, the CRISPR enzyme is a Cas9 enzyme. In some embodiments, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9 (e.g., modified to have or be associated with at least one DD), and may include further alteration or mutation of the Cas9, and can be a chimeric Cas9. In some embodiments, the DD-CRISPR enzyme is codon-optimized for expression in a eukaryotic cell. In some embodiments, the DD-CRISPR enzyme directs cleavage of one or two strands at the location of the target sequence. In some embodiments, the DD-CRISPR enzyme lacks or substantially DNA strand cleavage activity (e.g., no more than 5% nuclease activity as compared with a wild type enzyme or enzyme not having the mutation or alteration that decreases nuclease activity). In some embodiments, the first regulatory element is a polymerase III promoter. In some embodiments, the second regulatory element is a polymerase II promoter. In some embodiments, the guide sequence is at least 16, 17, 18, 19, 20, 25 nucleotides, or between 16-30, or between 16-25, or between 16-20 nucleotides in length.

In one aspect, the invention provides a method of modifying multiple target polynucleotides in a host cell such as a eukaryotic cell. In some embodiments, the method comprises allowing a Cas9CRISPR complex to bind to multiple target polynucleotides, e.g., to effect cleavage of said multiple target polynucleotides, thereby modifying multiple target polynucleotides, wherein the Cas9CRISPR complex comprises a Cas9 enzyme complexed with multiple guide sequences each of the being hybridized to a specific target sequence within said target polynucleotide, wherein said multiple guide sequences are linked to a direct repeat sequence. Where applicable, a tracr sequence may also be provided (e.g. to provide a single guide RNA, sgRNA). In some embodiments, said cleavage comprises cleaving one or two strands at the location of each of the target sequence by said Cas9 enzyme. In some embodiments, said cleavage results in decreased transcription of the multiple target genes. In some embodiments, the method further comprises repairing one or more of said cleaved target polynucleotide by homologous recombination with an exogenous template polynucleotide, wherein said repair results in a mutation comprising an insertion, deletion, or substitution of one or more nucleotides of one or more of said target polynucleotides. In some embodiments, said mutation results in one or more amino acid changes in a protein expressed from a gene comprising one or more of the target sequence(s). In some embodiments, the method further comprises delivering one or more vectors to said eukaryotic cell, wherein the one or more vectors drive expression of one or more of: the Cas9 enzyme and the multiple guide RNA sequence linked to a direct repeat sequence. Where applicable, a tracr sequence may also be provided. In some embodiments, said vectors are delivered to the eukaryotic cell in a subject. In some embodiments, said modifying takes place in said eukaryotic cell in a cell culture. In some embodiments, the method further comprises isolating said eukaryotic cell from a subject prior to said modifying. In some embodiments, the method further comprises returning said eukaryotic cell and/or cells derived therefrom to said subject.

In one aspect, the invention provides a method of modifying expression of multiple polynucleotides in a eukaryotic cell. In some embodiments, the method comprises allowing a Cas9 CRISPR complex to bind to multiple polynucleotides such that said binding results in increased or decreased expression of said polynucleotides; wherein the Cas9 CRISPR complex comprises a Cas9 enzyme complexed with multiple guide sequences each specifically hybridized to its own target sequence within said polynucleotide, wherein said guide sequences are linked to a direct repeat sequence. Where applicable, a tracr sequence may also be provided. In some embodiments, the method further comprises delivering one or more vectors to said eukaryotic cells, wherein the one or more vectors drive expression of one or more of: the Cas9 enzyme and the multiple guide sequences linked to the direct repeat sequences. Where applicable, a tracr sequence may also be provided.

In one aspect, the invention provides a recombinant polynucleotide comprising multiple guide RNA sequences up- or downstream (whichever applicable) of a direct repeat sequence, wherein each of the guide sequences when expressed directs sequence-specific binding of a Cas9CRISPR complex to its corresponding target sequence present in a eukaryotic cell. In some embodiments, the target sequence is a viral sequence present in a eukaryotic cell. Where applicable, a tracr sequence may also be provided. In some embodiments, the target sequence is a proto-oncogene or an oncogene.

Aspects of the invention encompass a non-naturally occurring or engineered composition that may comprise a guide RNA (gRNA) comprising a guide sequence capable of hybridizing to a target sequence in a genomic locus of interest in a cell and a Cas9 enzyme as defined herein that may comprise at least one or more nuclear localization sequences.

An aspect of the invention encompasses methods of modifying a genomic locus of interest to change gene expression in a cell by introducing into the cell any of the compositions described herein.

An aspect of the invention is that the above elements are comprised in a single composition or comprised in individual compositions. These compositions may advantageously be applied to a host to elicit a functional effect on the genomic level.

As used herein, the term “guide RNA” or “gRNA” has the leaning as used herein elsewhere and comprises any polynucleotide sequence having sufficient complementarity with a target nucleic acid sequence to hybridize with the target nucleic acid sequence and direct sequence-specific binding of a nucleic acid-targeting complex to the target nucleic acid sequence. Each gRNA may be designed to include multiple binding recognition sites (e.g., aptamers) specific to the same or different adapter protein. Each gRNA may be designed to bind to the promoter region −1000-+1 nucleic acids upstream of the transcription start site (i.e. TSS), preferably −200 nucleic acids. This positioning improves functional domains which affect gene activation (e.g., transcription activators) or gene inhibition (e.g., transcription repressors). The modified gRNA may be one or more modified gRNAs targeted to one or more target loci (e.g., at least 1 gRNA, at least 2 gRNA, at least 5 gRNA, at least 10 gRNA, at least 20 gRNA, at least 30 g RNA, at least 50 gRNA) comprised in a composition. Said multiple gRNA sequences can be tandemly arranged and are preferably separated by a direct repeat.

Thus, gRNA, the CRISPR enzyme as defined herein may each individually be comprised in a composition and administered to a host individually or collectively. Alternatively, these components may be provided in a single composition for administration to a host. Administration to a host may be performed via viral vectors known to the skilled person or described herein for delivery to a host (e.g., lentiviral vector, adenoviral vector, AAV vector). As explained herein, use of different selection markers (e.g., for lentiviral sgRNA selection) and concentration of gRNA (e.g., dependent on whether multiple gRNAs are used) may be advantageous for eliciting an improved effect. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. The compositions may be applied in a wide variety of methods for screening in libraries in cells and functional modeling in vivo (e.g., gene activation of lincRNA and identification of function; gain-of-function modeling; loss-of-function modeling; the use the compositions of the invention to establish cell lines and transgenic animals for optimization and screening purposes).

The current invention comprehends the use of the compositions of the current invention to establish and utilize conditional or inducible CRISPR transgenic cell/animals; see, e.g., Platt et al., Cell (2014), 159(2): 440-455, or PCT patent publications cited herein, such as WO 2014/093622 (PCT/US2013/074667). For example, cells or animals such as non-human animals, e.g., vertebrates or mammals, such as rodents, e.g., mice, rats, or other laboratory or field animals, e.g., cats, dogs, sheep, etc., may be ‘knock-in’ whereby the animal conditionally or inducibly expresses Cas9 akin to Platt et al. The target cell or animal thus comprises the CRISPR enzyme (e.g., Cas9) conditionally or inducibly (e.g., in the form of Cre dependent constructs), on expression of a vector introduced into the target cell, the vector expresses that which induces or gives rise to the condition of the CRISPR enzyme (e.g., Cas9) expression in the target cell. By applying the teaching and compositions as defined herein with the known method of creating a CRISPR complex, inducible genomic events are also an aspect of the current invention. Examples of such inducible events have been described herein elsewhere.

In some embodiments, phenotypic alteration is preferably the result of genome modification when a genetic disease is targeted, especially in methods of therapy and preferably where a repair template is provided to correct or alter the phenotype.

In some embodiments diseases that may be targeted include those concerned with disease-causing splice defects.

In some embodiments, cellular targets include Hemopoietic Stem/Progenitor Cells (CD34+); Human T cells; and Eye (retinal cells)—for example photoreceptor precursor cells.

In some embodiments Gene targets include: Human Beta Globin—HBB (for treating Sickle Cell Anemia, including by stimulating gene-conversion (using closely related HBD gene as an endogenous template)); CD3 (T-Cells); and CEP920-retina (eye).

In some embodiments disease targets also include: cancer; Sickle Cell Anemia (based on a point mutation); HBV, HIV; Beta-Thalassemia; and ophthalmic or ocular disease—for example Leber Congenital Amaurosis (LCA)-causing Splice Defect.

In some embodiments delivery methods include: Cationic Lipid Mediated “direct” delivery of Enzyme-Guide complex (RiboNucleoProtein) and electroporation of plasmid DNA.

Methods, products and uses described herein may be used for non-therapeutic purposes. Furthermore, any of the methods described herein may be applied in vitro and ex vivo.

In an aspect, provided is a non-naturally occurring or engineered composition comprising:

I. two or more CRISPR-Cas system polynucleotide sequences comprising

(a) a first guide sequence capable of hybridizing to a first target sequence in a polynucleotide locus,

(b) a second guide sequence capable of hybridizing to a second target sequence in a polynucleotide locus,

(c) a direct repeat sequence,

and

II. a Cas9 enzyme or a second polynucleotide sequence encoding it,

wherein when transcribed, the first and the second guide sequences direct sequence-specific binding of a first and a second Cas9 CRISPR complex to the first and second target sequences respectively,

wherein the first CRISPR complex comprises the Cas9 enzyme complexed with the first guide sequence that is hybridizable to the first target sequence,

wherein the second CRISPR complex comprises the Cas9 enzyme complexed with the second guide sequence that is hybridizable to the second target sequence, and

wherein the first guide sequence directs cleavage of one strand of the DNA duplex near the first target sequence and the second guide sequence directs cleavage of the other strand near the second target sequence inducing a double strand break, thereby modifying the organism or the non-human or non-animal organism. Similarly, compositions comprising more than two guide RNAs can be envisaged e.g. each specific for one target, and arranged tandemly in the composition or CRISPR system or complex as described herein.

In another embodiment, the Cas9 is delivered into the cell as a protein. In another and particularly preferred embodiment, the Cas9 is delivered into the cell as a protein or as a nucleotide sequence encoding it. Delivery to the cell as a protein may include delivery of a Ribonucleoprotein (RNP) complex, where the protein is complexed with the multiple guides.

In an aspect, host cells and cell lines modified by or comprising the compositions, systems or modified enzymes of present invention are provided, including stem cells, and progeny thereof.

In an aspect, methods of cellular therapy are provided, where, for example, a single cell or a population of cells is sampled or cultured, wherein that cell or cells is or has been modified ex vivo as described herein, and is then re-introduced (sampled cells) or introduced (cultured cells) into the organism. Stem cells, whether embryonic or induce pluripotent or totipotent stem cells, are also particularly preferred in this regard. But, of course, in vivo embodiments are also envisaged.

Inventive methods can further comprise delivery of templates, such as repair templates, which may be dsODN or ssODN, see below. Delivery of templates may be via the cotemporaneous or separate from delivery of any or all the CRISPR enzyme or guide RNAs and via the same delivery mechanism or different. In some embodiments, it is preferred that the template is delivered together with the guide RNAs and, preferably, also the CRISPR enzyme. An example may be an AAV vector where the CRISPR enzyme is AsCas9 or LbCas9.

Inventive methods can further comprise: (a) delivering to the cell a double-stranded oligodeoxynucleotide (dsODN) comprising overhangs complimentary to the overhangs created by said double strand break, wherein said dsODN is integrated into the locus of interest; or—(b) delivering to the cell a single-stranded oligodeoxynucleotide (ssODN), wherein said ssODN acts as a template for homology directed repair of said double strand break. Inventive methods can be for the prevention or treatment of disease in an individual, optionally wherein said disease is caused by a defect in said locus of interest. Inventive methods can be conducted in vivo in the individual or ex vivo on a cell taken from the individual, optionally wherein said cell is returned to the individual.

The invention also comprehends products obtained from using CRISPR enzyme or Cas enzyme or Cas9 enzyme or CRISPR-CRISPR enzyme or CRISPR-Cas system or CRISPR-Cas9 system for use in tandem or multiple targeting as defined herein.

### Escorted Guides for the Cas9 CRISPR-Cas System According to the Invention

In one aspect the invention provides escorted Cas9 CRISPR-Cas systems or complexes, especially such a system involving an escorted Cas9 CRISPR-Cas system guide. By “escorted” is meant that the Cas9 CRISPR-Cas system or complex or guide is delivered to a selected time or place within a cell, so that activity of the Cas9 CRISPR-Cas system or complex or guide is spatially or temporally controlled. For example, the activity and destination of the Cas9 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted Cas9 CRISPR-Cas systems or complexes have a gRNA with a functional structure designed to improve gRNA structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, provided herein is a gRNA modified, e.g., by one or more aptamer(s) designed to improve gRNA delivery, including delivery across the cellular membrane, to intracellular compartments, or into the nucleus. Such a structure can include, either in addition to the one or more aptamer(s) or without such one or more aptamer(s), moiety(ies) so as to render the guide deliverable, inducible or responsive to a selected effector. The invention accordingly comprehends an gRNA that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

An aspect of the invention provides non-naturally occurring or engineered composition comprising an escorted guide RNA (egRNA) comprising:
an RNA guide sequence capable of hybridizing to a target sequence in a genomic locus of interest in a cell; and,
an escort RNA aptamer sequence, wherein the escort aptamer has binding affinity for an aptamer ligand on or in the cell, or the escort aptamer is responsive to a localized aptamer effector on or in the cell, wherein the presence of the aptamer ligand or effector on or in the cell is spatially or temporally restricted.

The escort aptamer may for example change conformation in response to an interaction with the aptamer ligand or effector in the cell.

The escort aptamer may have specific binding affinity for the aptamer ligand.

The aptamer ligand may be localized in a location or compartment of the cell, for example on or in a membrane of the cell. Binding of the escort aptamer to the aptamer ligand may accordingly direct the egRNA to a location of interest in the cell, such as the interior of the cell by way of binding to an aptamer ligand that is a cell surface ligand. In this way, a variety of spatially restricted locations within the cell may be targeted, such as the cell nucleus or mitochondria.

Once intended alterations have been introduced, such as by editing intended copies of a gene in the genome of a cell, continued CRISPR/Cas9 expression in that cell is no longer necessary. Indeed, sustained expression would be undesirable in certain casein case of off-target effects at unintended genomic sites, etc. Thus time-limited expression would be useful. Inducible expression offers one approach, but in addition Applicants have engineered a Self-Inactivating Cas9 CRISPR-Cas system that relies on the use of a non-coding guide target sequence within the CRISPR vector itself. Thus, after expression begins, the CRISPR system will lead to its own destruction, but before destruction is complete it will have time to edit the genomic copies of the target gene (which, with a normal point mutation in a diploid cell, requires at most two edits). Simply, the self inactivating Cas9 CRISPR-Cas system includes additional RNA (i.e., guide RNA) that targets the coding sequence for the CRISPR enzyme itself or that targets one or more non-coding guide target sequences complementary to unique sequences present in one or more of the following: (a) within the promoter driving expression of the non-coding RNA elements, (b) within the promoter driving expression of the Cas9 gene, (c) within 100 bp of the ATG translational start codon in the Cas9 coding sequence, (d) within the inverted terminal repeat (iTR) of a viral delivery vector, e.g., in an AAV genome.

The egRNA may include an RNA aptamer linking sequence, operably linking the escort RNA sequence to the RNA guide sequence.

In embodiments, the egRNA may include one or more photolabile bonds or non-naturally occurring residues.

In one aspect, the escort RNA aptamer sequence may be complementary to a target miRNA, which may or may not be present within a cell, so that only when the target miRNA is present is there binding of the escort RNA aptamer sequence to the target miRNA which results in cleavage of the egRNA by an RNA-induced silencing complex (RISC) within the cell.

In embodiments, the escort RNA aptamer sequence may for example be from 10 to 200 nucleotides in length, and the egRNA may include more than one escort RNA aptamer sequence.

It is to be understood that any of the RNA guide sequences as described herein elsewhere can be used in the egRNA described herein. In certain embodiments of the invention, the guide RNA or mature crRNA comprises, consists essentially of, or consists of a direct repeat sequence and a guide sequence or spacer sequence. In certain embodiments, the guide RNA or mature crRNA comprises, consists essentially of, or consists of a direct repeat sequence linked to a guide sequence or spacer sequence. In certain embodiments the guide RNA or mature crRNA comprises 19 nts of partial direct repeat followed by 23-25 nt of guide sequence or spacer sequence. In certain embodiments, the effector protein is a FnCas9 effector protein and requires at least 16 nt of guide sequence to achieve detectable DNA cleavage and a minimum of 17 nt of guide sequence to achieve efficient DNA cleavage in vitro. In certain embodiments, the direct repeat sequence is located upstream (i.e., 5′) from the guide sequence or spacer sequence. In a preferred embodiment the seed sequence (i.e. the sequence essential critical for recognition and/or hybridization to the sequence at the target locus) of the FnCas9 guide RNA is approximately within the first 5 nt on the 5′ end of the guide sequence or spacer sequence.

The egRNA may be included in a non-naturally occurring or engineered Cas9 CRISPR-Cas complex composition, together with a Cas9 which may include at least one mutation, for example a mutation so that the Cas9 has no more than 5% of the nuclease activity of a Cas9 not having the at least one mutation, for example having a diminished nuclease activity of at least 97%, or 100% as compared with the Cas9 not having the at least one mutation. The Cas9 may also include one or more nuclear localization sequences. Mutated Cas9 enzymes having modulated activity such as diminished nuclease activity are described herein elsewhere.

The engineered Cas9 CRISPR-Cas composition may be provided in a cell, such as a eukaryotic cell, a mammalian cell, or a human cell.

In embodiments, the compositions described herein comprise a Cas9 CRISPR-Cas complex having at least three functional domains, at least one of which is associated with Cas9 and at least two of which are associated with egRNA.

The compositions described herein may be used to introduce a genomic locus event in a host cell, such as an eukaryotic cell, in particular a mammalian cell, or a non-human eukaryote, in particular a non-human mammal such as a mouse, in vivo. The genomic locus event may comprise affecting gene activation, gene inhibition, or cleavage in a locus. The compositions described herein may also be used to modify a genomic locus of interest to change gene expression in a cell. Methods of introducing a genomic locus event in a host cell using the Cas9 enzyme provided herein are described herein in detail elsewhere. Delivery of the composition may for example be by way of delivery of a nucleic acid molecule(s) coding for the composition, which nucleic acid molecule(s) is operatively linked to regulatory sequence(s), and expression of the nucleic acid molecule(s) in vivo, for example by way of a lentivirus, an adenovirus, or an AAV.

The present invention provides compositions and methods by which gRNA-mediated gene editing activity can be adapted. The invention provides gRNA secondary structures that improve cutting efficiency by increasing gRNA and/or increasing the amount of RNA delivered into the cell. The gRNA may include light labile or inducible nucleotides.

To increase the effectiveness of gRNA, for example gRNA delivered with viral or non-viral technologies, Applicants added secondary structures into the gRNA that enhance its stability and improve gene editing. Separately, to overcome the lack of effective delivery, Applicants modified gRNAs with cell penetrating RNA aptamers; the aptamers bind to cell surface receptors and promote the entry of gRNAs into cells. Notably, the cell-penetrating aptamers can be designed to target specific cell receptors, in order to mediate cell-specific delivery. Applicants also have created guides that are inducible.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

Cells involved in the practice of the present invention may be a prokaryotic cell or a eukaryotic cell, advantageously an animal cell a plant cell or a yeast cell, more advantageously a mammalian cell.

The chemical or energy sensitive guide may undergo a conformational change upon induction by the binding of a chemical source or by the energy allowing it act as a guide and have the Cas9 CRISPR-Cas system or complex function. The invention can involve applying the chemical source or energy so as to have the guide function and the Cas9 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., http://stke.sciencemag.org/cgi/content/abstract/sigtrans;4/164/rs2), 2. FKBP-FRB based system inducible by rapamycin (or related chemicals based on rapamycin) (see, e.g., http://www.nature.com/nmeth/journal/v2/n6/full/nmeth763.html), 3. GID1-GAI based system inducible by Gibberellin (GA) (see, e.g., http://www.nature.com/nchembio/journal/v8/n5/full/nchembio.922.html).

Another system contemplated by the present invention is a chemical inducible system based on change in sub-cellular localization. Applicants also developed a system in which the polypeptide include a DNA binding domain comprising at least five or more Transcription activator-like effector (TALE) monomers and at least one or more half-monomers specifically ordered to target the genomic locus of interest linked to at least one or more effector domains are further linker to a chemical or energy sensitive protein. This protein will lead to a change in the sub-cellular localization of the entire polypeptide (i.e. transportation of the entire polypeptide from cytoplasm into the nucleus of the cells) upon the binding of a chemical or energy transfer to the chemical or energy sensitive protein. This transportation of the entire polypeptide from one sub-cellular compartments or organelles, in which its activity is sequestered due to lack of substrate for the effector domain, into another one in which the substrate is present would allow the entire polypeptide to come in contact with its desired substrate (i.e. genomic DNA in the mammalian nucleus) and result in activation or repression of target gene expression.

This type of system could also be used to induce the cleavage of a genomic locus of interest in a cell when the effector domain is a nuclease.

A chemical inducible system can be an estrogen receptor (ER) based system inducible by 4-hydroxytamoxifen (40HT) (see, e.g., http://www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., http://www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas9 CRISPR-Cas complex or system, and the binding will induce the change of sub-cellular localization of the polypeptide, leading to the entire polypeptide entering the nucleus of cells. Once inside the nucleus, the guide protein and the other components of the Cas9 CRISPR-Cas complex will be active and modulating target gene expression in cells.

This type of system could also be used to induce the cleavage of a genomic locus of interest in a cell; and, in this regard, it is noted that the Cas9 enzyme is a nuclease. The light could be generated with a laser or other forms of energy sources. The heat could be generated by raise of temperature results from an energy source, or from nano-particles that release heat after absorbing energy from an energy source delivered in the form of radio-wave.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100 .mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm² to about 100 W/cm². Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm² (FDA recommendation), although energy densities of up to 750 mW/cm² have been used. In physiotherapy, ultrasound is typically used as an energy source in a range up to about 3 to 4 W/cm² (WHO recommendation). In other therapeutic applications, higher intensities of ultrasound may be employed, for example, HIFU at 100 W/cm up to 1 kW/cm² (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm⁻². Even more preferably, the exposure to an ultrasound energy source is at a power density of from about 1 to about 15 Wcm⁻².

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm⁻² to about 10 Wcm⁻² with a frequency ranging from about 0.015 to about 10 MHz (see WO 98/52609). However, alternatives are also possible, for example, exposure to an ultrasound energy source at an acoustic power density of above 100 Wcm⁻², but for reduced periods of time, for example, 1000 Wcm⁻² for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm⁻² or 1.25 Wcm⁻² as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

The rapid transcriptional response and endogenous targeting of the instant invention make for an ideal system for the study of transcriptional dynamics. For example, the instant invention may be used to study the dynamics of variant production upon induced expression of a target gene. On the other end of the transcription cycle, mRNA degradation studies are often performed in response to a strong extracellular stimulus, causing expression level changes in a plethora of genes. The instant invention may be utilized to reversibly induce transcription of an endogenous target, after which point stimulation may be stopped and the degradation kinetics of the unique target may be tracked.

The temporal precision of the instant invention may provide the power to time genetic regulation in concert with experimental interventions. For example, targets with suspected involvement in long-term potentiation (LTP) may be modulated in organotypic or dissociated neuronal cultures, but only during stimulus to induce LTP, so as to avoid interfering with the normal development of the cells. Similarly, in cellular models exhibiting disease phenotypes, targets suspected to be involved in the effectiveness of a particular therapy may be modulated only during treatment. Conversely, genetic targets may be modulated only during a pathological stimulus. Any number of experiments in which timing of genetic cues to external experimental stimuli is of relevance may potentially benefit from the utility of the instant invention.

The in vivo context offers equally rich opportunities for the instant invention to control gene expression. Photoinducibility provides the potential for spatial precision. Taking advantage of the development of optrode technology, a stimulating fiber optic lead may be placed in a precise brain region. Stimulation region size may then be tuned by light intensity. This may be done in conjunction with the delivery of the Cas9 CRISPR-Cas system or complex of the invention, or, in the case of transgenic Cas9 animals, guide RNA of the invention may be delivered and the optrode technology can allow for the modulation of gene expression in precise brain regions. A transparent Cas9 expressing organism, can have guide RNA of the invention administered to it and then there can be extremely precise laser induced local gene expression changes.

A culture medium for culturing host cells includes a medium commonly used for tissue culture, such as M199-earle base, Eagle MEM (E-MEM), Dulbecco MEM (DMEM), SC-UCM102, UP-SFM (GIBCO BRL), EX-CELL302 (Nichirei), EX-CELL293-S(Nichirei), TFBM-01 (Nichirei), ASF104, among others. Suitable culture media for specific cell types may be found at the American Type Culture Collection (ATCC) or the European Collection of Cell Cultures (ECACC). Culture media may be supplemented with amino acids such as L-glutamine, salts, anti-fungal or anti-bacterial agents such as Fungizone®, penicillin-streptomycin, animal serum, and the like. The cell culture medium may optionally be serum-free.

The invention may also offer valuable temporal precision in vivo. The invention may be used to alter gene expression during a particular stage of development. The invention may be used to time a genetic cue to a particular experimental window. For example, genes implicated in learning may be overexpressed or repressed only during the learning stimulus in a precise region of the intact rodent or primate brain. Further, the invention may be used to induce gene expression changes only during particular stages of disease development. For example, an oncogene may be overexpressed only once a tumor reaches a particular size or metastatic stage. Conversely, proteins suspected in the development of Alzheimer's may be knocked down only at defined time points in the animal's life and within a particular brain region. Although these examples do not exhaustively list the potential applications of the invention, they highlight some of the areas in which the invention may be a powerful technology.

Protected Guides: Cas Proteins According to the Invention can be Used in Combination with Protected Guide RNAs

In one aspect, an object of the current invention is to further enhance the specificity of Cas9 given individual guide RNAs through thermodynamic tuning of the binding specificity of the guide RNA to target DNA. This is a general approach of introducing mismatches, elongation or truncation of the guide sequence to increase/decrease the number of complimentary bases vs. mismatched bases shared between a genomic target and its potential off-target loci, in order to give thermodynamic advantage to targeted genomic loci over genomic off-targets.

In one aspect, the invention provides for the guide sequence being modified by secondary structure to increase the specificity of the Cas9 CRISPR-Cas system and whereby the secondary structure can protect against exonuclease activity and allow for 3′ additions to the guide sequence.

In one aspect, the invention provides for hybridizing a “protector RNA” to a guide sequence, wherein the “protector RNA” is an RNA strand complementary to the 5′ end of the guide RNA (gRNA), to thereby generate a partially double-stranded gRNA. In an embodiment of the invention, protecting the mismatched bases with a perfectly complementary protector sequence decreases the likelihood of target DNA binding to the mismatched base pairs at the 3′ end. In embodiments of the invention, additional sequences comprising an extended length may also be present.

Guide RNA (gRNA) extensions matching the genomic target provide gRNA protection and enhance specificity. Extension of the gRNA with matching sequence distal to the end of the spacer seed for individual genomic targets is envisaged to provide enhanced specificity. Matching gRNA extensions that enhance specificity have been observed in cells without truncation. Prediction of gRNA structure accompanying these stable length extensions has shown that stable forms arise from protective states, where the extension forms a closed loop with the gRNA seed due to complimentary sequences in the spacer extension and the spacer seed. These results demonstrate that the protected guide concept also includes sequences matching the genomic target sequence distal of the 20mer spacer-binding region. Thermodynamic prediction can be used to predict completely matching or partially matching guide extensions that result in protected gRNA states. This extends the concept of protected gRNAs to interaction between X and Z, where X will generally be of length 17-20 nt and Z is of length 1-30 nt. Thermodynamic prediction can be used to determine the optimal extension state for Z, potentially introducing small numbers of mismatches in Z to promote the formation of protected conformations between X and Z. Throughout the present application, the terms “X” and seed length (SL) are used interchangeably with the term exposed length (EpL) which denotes the number of nucleotides available for target DNA to bind; the terms “Y” and protector length (PL) are used interchangeably to represent the length of the protector; and the terms “Z”, “E”, “E′” and “EL” are used interchangeably to correspond to the term extended length (ExL) which represents the number of nucleotides by which the target sequence is extended.

An extension sequence which corresponds to the extended length (ExL) may optionally be attached directly to the guide sequence at the 3′ end of the protected guide sequence. The extension sequence may be 2 to 12 nucleotides in length. Preferably ExL may be denoted as 0, 2, 4, 6, 8, 10 or 12 nucleotides in length. In a preferred embodiment the ExL is denoted as 0 or 4 nucleotides in length. In a more preferred embodiment the ExL is 4 nucleotides in length. The extension sequence may or may not be complementary to the target sequence.

An extension sequence may further optionally be attached directly to the guide sequence at the 5′ end of the protected guide sequence as well as to the 3′ end of a protecting sequence. As a result, the extension sequence serves as a linking sequence between the protected sequence and the protecting sequence. Without wishing to be bound by theory, such a link may position the protecting sequence near the protected sequence for improved binding of the protecting sequence to the protected sequence. It will be understood that the above-described relationship of seed, protector, and extension applies where the distal end (i.e., the targeting end) of the guide is the 5′ end, e.g. a guide that functions is a Cas9 system. In an embodiment wherein the distal end of the guide is the 3′ end, the relationship will be the reverse. In such an embodiment, the invention provides for hybridizing a “protector RNA” to a guide sequence, wherein the “protector RNA” is an RNA strand complementary to the 3′ end of the guide RNA (gRNA), to thereby generate a partially double-stranded gRNA.

Addition of gRNA mismatches to the distal end of the gRNA can demonstrate enhanced specificity. The introduction of unprotected distal mismatches in Y or extension of the gRNA with distal mismatches (Z) can demonstrate enhanced specificity. This concept as mentioned is tied to X, Y, and Z components used in protected gRNAs. The unprotected mismatch concept may be further generalized to the concepts of X, Y, and Z described for protected guide RNAs.

Cas9. In one aspect, the invention provides for enhanced Cas9 specificity wherein the double stranded 3′ end of the protected guide RNA (pgRNA) allows for two possible outcomes: (1) the guide RNA-protector RNA to guide RNA-target DNA strand exchange will occur and the guide will fully bind the target, or (2) the guide RNA will fail to fully bind the target and because Cas9 target cleavage is a multiple step kinetic reaction that requires guide RNA:target DNA binding to activate Cas9-catalyzed DSBs, wherein Cas9 cleavage does not occur if the guide RNA does not properly bind. According to particular embodiments, the protected guide RNA improves specificity of target binding as compared to a naturally occurring CRISPR-Cas system. According to particular embodiments the protected modified guide RNA improves stability as compared to a naturally occurring CRISPR-Cas. According to particular embodiments the protector sequence has a length between 3 and 120 nucleotides and comprises 3 or more contiguous nucleotides complementary to another sequence of guide or protector. According to particular embodiments, the protector sequence forms a hairpin. According to particular embodiments the guide RNA further comprises a protected sequence and an exposed sequence. According to particular embodiments the exposed sequence is 1 to 19 nucleotides. More particularly, the exposed sequence is at least 75%, at least 90% or about 100% complementary to the target sequence. According to particular embodiments the guide sequence is at least 90% or about 100% complementary to the protector strand. According to particular embodiments the guide sequence is at least 75%, at least 90% or about 100% complementary to the target sequence. According to particular embodiments, the guide RNA further comprises an extension sequence. More particularly, when the distal end of the guide is the 3′ end, the extension sequence is operably linked to the 3′ end of the protected guide sequence, and optionally directly linked to the 3′ end of the protected guide sequence. According to particular embodiments the extension sequence is 1-12 nucleotides. According to particular embodiments the extension sequence is operably linked to the guide sequence at the 3′ end of the protected guide sequence and the 5′ end of the protector strand and optionally directly linked to the 3′ end of the protected guide sequence and the 5′ end of the protector strand, wherein the extension sequence is a linking sequence between the protected sequence and the protector strand. According to particular embodiments the extension sequence is 100% not complementary to the protector strand, optionally at least 95%, at least 90%, at least 80%, at least 70%, at least 60%, or at least 50% not complementary to the protector strand. According to particular embodiments the guide sequence further comprises mismatches appended to the end of the guide sequence, wherein the mismatches thermodynamically optimize specificity.

According to the invention, in certain embodiments, guide modifications that impede strand invasion will be desireable. For example, to minimize off-target actifity, in certain embodiments, it will be desireable to design or modify a guide to impede strand invasiom at off-target sites. In certain such embodiments, it may be acceptable or useful to design or modify a guide at the expense of on-target binding efficiency. In certain embodiments, guide-target mismatches at the target site may be tolerated that substantially reduce off-target activity.

In certain embodiments of the invention, it is desirable to adjust the binding characteristics of the protected guide to minimize off-target CRISPR activity. Accordingly, thermodynamic prediction algoithms are used to predict strengths of binding on target and off target. Alternatively or in addition, selection methods are used to reduce or minimize off-target effects, by absolute measures or relative to on-target effects.

Design options include, without limitation, i) adjusting the length of protector strand that binds to the protected strand, ii) adjusting the length of the portion of the protected strand that is exposed, iii) extending the protected strand with a stem-loop located external (distal) to the protected strand (i.e. designed so that the stem loop is external to the protected strand at the distal end), iv) extending the protected strand by addition of a protector strand to form a stem-loop with all or part of the protected strand, v) adjusting binding of the protector strand to the protected strand by designing in one or more base mismatches and/or one or more non-canonical base pairings, vi) adjusting the location of the stem formed by hybridization of the protector strand to the protected strand, and vii) addition of a non-structured protector to the end of the protected strand.

In one aspect, the invention provides an engineered, non-naturally occurring CRISPR-Cas system comprising a Cas protein and a protected guide RNA that targets a DNA molecule encoding a gene product in a cell, whereby the protected guide RNA targets the DNA molecule encoding the gene product and the Cas protein cleaves the DNA molecule encoding the gene product, whereby expression of the gene product is altered; and, wherein the Cas9 protein and the protected guide RNA do not naturally occur together. The invention comprehends the protected guide RNA comprising a guide sequence fused to a direct repeat sequence. The invention further comprehends the CRISPR protein being codon optimized for expression in a eukaryotic cell. In a preferred embodiment the eukaryotic cell is a mammalian cell, a plant cell or a yeast cell and in a more preferred embodiment the mammalian cell is a human cell. In a further embodiment of the invention, the expression of the gene product is decreased. In some embodiments the CRISPR protein is Cas12 or Cas13. In some embodiments the CRISPR protein is Cas12a. In some embodiments, the Cas12a protein is Acidaminococcus sp. BV3L6, Lachnospiraceae bacterium or Francisella Novicida Cas12a, and may include mutated Cas12a derived from these organisms. The protein may be a further Cas12a homolog or ortholog. In some embodiments, the nucleotide sequence encoding the Cas protein is codon-optimized for expression in a eukaryotic cell. In some embodiments, the Cas9 or Cas12a protein directs cleavage of one or two strands at the location of the target sequence. In some embodiments, the first regulatory element is a polymerase III promoter. In some embodiments, the second regulatory element is a polymerase II promoter. In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

Advantageous vectors include lentiviruses and adeno-associated viruses, and types of such vectors can also be selected for targeting particular types of cells.

In one aspect, the invention provides a eukaryotic host cell comprising (a) a first regulatory element operably linked to a direct repeat sequence and one or more insertion sites for inserting one or more guide sequences downstream of the direct repeat sequence, wherein when expressed, the guide sequence directs sequence-specific binding of a CRISPR complex to a target sequence in a eukaryotic cell, wherein the CRISPR complex comprises a CRISPR enzyme complexed with the guide RNA comprising the guide sequence that is hybridized to the target sequence and/or (b) a second regulatory element operably linked to an enzyme-coding sequence encoding said Cas9 enzyme comprising a nuclear localization sequence. In some embodiments, the host cell comprises components (a) and (b). In some embodiments, component (a), component (b), or components (a) and (b) are stably integrated into a genome of the host eukaryotic cell. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the Cas9 enzyme directs cleavage of one or two strands at the location of the target sequence. In some embodiments, the Cas9 enzyme lacks DNA strand cleavage activity. In some embodiments, the first regulatory element is a polymerase III promoter. In some embodiments, the second regulatory element is a polymerase II promoter.

In an aspect, the invention provides a non-human eukaryotic organism; preferably a multicellular eukaryotic organism, comprising a eukaryotic host cell according to any of the described embodiments. In other aspects, the invention provides a eukaryotic organism; preferably a multicellular eukaryotic organism, comprising a eukaryotic host cell according to any of the described embodiments. The organism in some embodiments of these aspects may be an animal; for example a mammal. Also, the organism may be an arthropod such as an insect. The organism also may be a plant or a yeast. Further, the organism may be a fungus.

In one aspect, the invention provides a kit comprising one or more of the components described herein above. In some embodiments, the kit comprises a vector system and instructions for using the kit. In some embodiments, the vector system comprises (a) a first regulatory element operably linked to a direct repeat sequence and one or more insertion sites for inserting one or more guide sequences downstream of the direct repeat sequence, wherein when expressed, the guide sequence directs sequence-specific binding of a Cas9 CRISPR complex to a target sequence in a eukaryotic cell, wherein the CRISPR complex comprises a Cas9 enzyme complexed with the protected guide RNA comprising the guide sequence that is hybridized to the target sequence and/or (b) a second regulatory element operably linked to an enzyme-coding sequence encoding said Cas9 enzyme comprising a nuclear localization sequence. In some embodiments, the kit comprises components (a) and (b) located on the same or different vectors of the system. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the Cas9 enzyme comprises one or more nuclear localization sequences of sufficient strength to drive accumulation of said Cas9 enzyme in a detectable amount in the nucleus of a eukaryotic cell. In some embodiments, the Cas9 enzyme is Acidaminococcus sp. BV3L6, Lachnospiraceae bacterium MA2020 or Francisella tularensis 1 Novicida Cas9, and may include mutated Cas9 derived from these organisms. The enzyme may be a Cas9 homolog or ortholog. In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity. In some embodiments, the first regulatory element is a polymerase III promoter. In some embodiments, the second regulatory element is a polymerase II promoter.

In one aspect, the invention provides a method of modifying a target polynucleotide in a eukaryotic cell. In some embodiments, the method comprises allowing a CRISPR complex to bind to the target polynucleotide to effect cleavage of said target polynucleotide thereby modifying the target polynucleotide, wherein the CRISPR complex comprises a Cas9 enzyme complexed with protected guide RNA comprising a guide sequence hybridized to a target sequence within said target polynucleotide. In some embodiments, said cleavage comprises cleaving one or two strands at the location of the target sequence by said Cas9 enzyme. In some embodiments, said cleavage results in decreased transcription of a target gene. In some embodiments, the method further comprises repairing said cleaved target polynucleotide by non-homologous end joining (NHEJ)-based gene insertion mechanisms, more particularly with an exogenous template polynucleotide, wherein said repair results in a mutation comprising an insertion, deletion, or substitution of one or more nucleotides of said target polynucleotide. In some embodiments, said mutation results in one or more amino acid changes in a protein expressed from a gene comprising the target sequence. In some embodiments, the method further comprises delivering one or more vectors to said eukaryotic cell, wherein the one or more vectors drive expression of one or more of: the Cas9 enzyme, the protected guide RNA comprising the guide sequence linked to direct repeat sequence. In some embodiments, said vectors are delivered to the eukaryotic cell in a subject. In some embodiments, said modifying takes place in said eukaryotic cell in a cell culture. In some embodiments, the method further comprises isolating said eukaryotic cell from a subject prior to said modifying. In some embodiments, the method further comprises returning said eukaryotic cell and/or cells derived therefrom to said subject.

In one aspect, the invention provides a method of modifying expression of a polynucleotide in a eukaryotic cell. In some embodiments, the method comprises allowing a Cas9 CRISPR complex to bind to the polynucleotide such that said binding results in increased or decreased expression of said polynucleotide; wherein the CRISPR complex comprises a Cas9 enzyme complexed with a protected guide RNA comprising a guide sequence hybridized to a target sequence within said polynucleotide. In some embodiments, the method further comprises delivering one or more vectors to said eukaryotic cells, wherein the one or more vectors drive expression of one or more of: the Cas9 enzyme and the protected guide RNA.

In one aspect, the invention provides a method of generating a model eukaryotic cell comprising a mutated disease gene. In some embodiments, a disease gene is any gene associated an increase in the risk of having or developing a disease. In some embodiments, the method comprises (a) introducing one or more vectors into a eukaryotic cell, wherein the one or more vectors drive expression of one or more of: a Cas9 enzyme and a protected guide RNA comprising a guide sequence linked to a direct repeat sequence; and (b) allowing a CRISPR complex to bind to a target polynucleotide to effect cleavage of the target polynucleotide within said disease gene, wherein the CRISPR complex comprises the Cas9 enzyme complexed with the guide RNA comprising the sequence that is hybridized to the target sequence within the target polynucleotide, thereby generating a model eukaryotic cell comprising a mutated disease gene. In some embodiments, said cleavage comprises cleaving one or two strands at the location of the target sequence by said Cas9 enzyme. In some embodiments, said cleavage results in decreased transcription of a target gene. In some embodiments, the method further comprises repairing said cleaved target polynucleotide by non-homologous end joining (NHEJ)-based gene insertion mechanisms with an exogenous template polynucleotide, wherein said repair results in a mutation comprising an insertion, deletion, or substitution of one or more nucleotides of said target polynucleotide. In some embodiments, said mutation results in one or more amino acid changes in a protein expression from a gene comprising the target sequence.

In one aspect, the invention provides a method for developing a biologically active agent that modulates a cell signaling event associated with a disease gene. In some embodiments, a disease gene is any gene associated an increase in the risk of having or developing a disease. In some embodiments, the method comprises (a) contacting a test compound with a model cell of any one of the described embodiments; and (b) detecting a change in a readout that is indicative of a reduction or an augmentation of a cell signaling event associated with said mutation in said disease gene, thereby developing said biologically active agent that modulates said cell signaling event associated with said disease gene.

In one aspect, the invention provides a recombinant polynucleotide comprising a protected guide sequence downstream of a direct repeat sequence, wherein the protected guide sequence when expressed directs sequence-specific binding of a CRISPR complex to a corresponding target sequence present in a eukaryotic cell. In some embodiments, the target sequence is a viral sequence present in a eukaryotic cell. In some embodiments, the target sequence is a proto-oncogene or an oncogene.

In one aspect the invention provides for a method of selecting one or more cell(s) by introducing one or more mutations in a gene in the one or more cell (s), the method comprising: introducing one or more vectors into the cell (s), wherein the one or more vectors drive expression of one or more of: a Cas9 enzyme, a protected guide RNA comprising a guide sequence, and an editing template; wherein the editing template comprises the one or more mutations that abolish Cas9 enzyme cleavage; allowing non-homologous end joining (NHEJ)-based gene insertion mechanisms of the editing template with the target polynucleotide in the cell(s) to be selected; allowing a CRISPR complex to bind to a target polynucleotide to effect cleavage of the target polynucleotide within said gene, wherein the CRISPR complex comprises the Cas9 enzyme complexed with the protected guide RNA comprising a guide sequence that is hybridized to the target sequence within the target polynucleotide, wherein binding of the CRISPR complex to the target polynucleotide induces cell death, thereby allowing one or more cell(s) in which one or more mutations have been introduced to be selected. In a preferred embodiment of the invention the cell to be selected may be a eukaryotic cell. Aspects of the invention allow for selection of specific cells without requiring a selection marker or a two-step process that may include a counter-selection system.

With respect to mutations of the Cas9 enzyme, when the enzyme is not FnCas9, mutations may be as described herein elsewhere; conservative substitution for any of the replacement amino acids is also envisaged. In an aspect the invention provides as to any or each or all embodiments herein-discussed wherein the CRISPR enzyme comprises at least one or more, or at least two or more mutations, wherein the at least one or more mutation or the at least two or more mutations are selected from those described herein elsewhere.

In a further aspect, the invention involves a computer-assisted method for identifying or designing potential compounds to fit within or bind to CRISPR-Cas9 system or a functional portion thereof or vice versa (a computer-assisted method for identifying or designing potential CRISPR-Cas9 systems or a functional portion thereof for binding to desired compounds) or a computer-assisted method for identifying or designing potential CRISPR-Cas9 systems (e.g., with regard to predicting areas of the CRISPR-Cas9 system to be able to be manipulated—for instance, based on crystal structure data or based on data of Cas9 orthologs, or with respect to where a functional group such as an activator or repressor can be attached to the CRISPR-Cas9 system, or as to Cas9 truncations or as to designing nickases), said method comprising:

using a computer system, e.g., a programmed computer comprising a processor, a data storage system, an input device, and an output device, the steps of:

(a) inputting into the programmed computer through said input device data comprising the three-dimensional co-ordinates of a subset of the atoms from or pertaining to the CRISPR-Cas9 crystal structure, e.g., in the CRISPR-Cas9 system binding domain or alternatively or additionally in domains that vary based on variance among Cas9 orthologs or as to Cas9s or as to nickases or as to functional groups, optionally with structural information from CRISPR-Cas9 system complex(es), thereby generating a data set;

(b) comparing, using said processor, said data set to a computer database of structures stored in said computer data storage system, e.g., structures of compounds that bind or putatively bind or that are desired to bind to a CRISPR-Cas9 system or as to Cas9 orthologs (e.g., as Cas9s or as to domains or regions that vary amongst Cas9 orthologs) or as to the CRISPR-Cas9 crystal structure or as to nickases or as to functional groups;

(c) selecting from said database, using computer methods, structure(s)—e.g., CRISPR-Cas9 structures that may bind to desired structures, desired structures that may bind to certain CRISPR-Cas9 structures, portions of the CRISPR-Cas9 system that may be manipulated, e.g., based on data from other portions of the CRISPR-Cas9 crystal structure and/or from Cas9 orthologs, truncated Cas9s, novel nickases or particular functional groups, or positions for attaching functional groups or functional-group-CRISPR-Cas9 systems;

(d) constructing, using computer methods, a model of the selected structure(s); and

(e) outputting to said output device the selected structure(s);

and optionally synthesizing one or more of the selected structure(s);

and further optionally testing said synthesized selected structure(s) as or in a CRISPR-Cas9 system;

or, said method comprising: providing the co-ordinates of at least two atoms of the CRISPR-Cas9 crystal structure, e.g., at least two atoms of the herein Crystal Structure Table of the CRISPR-Cas9 crystal structure or co-ordinates of at least a sub-domain of the CRISPR-Cas9 crystal structure (“selected co-ordinates”), providing the structure of a candidate comprising a binding molecule or of portions of the CRISPR-Cas9 system that may be manipulated, e.g., based on data from other portions of the CRISPR-Cas9 crystal structure and/or from Cas9 orthologs, or the structure of functional groups, and fitting the structure of the candidate to the selected co-ordinates, to thereby obtain product data comprising CRISPR-Cas9 structures that may bind to desired structures, desired structures that may bind to certain CRISPR-Cas9 structures, portions of the CRISPR-Cas9 system that may be manipulated, truncated Cas9s, novel nickases, or particular functional groups, or positions for attaching functional groups or functional-group-CRISPR-Cas9 systems, with output thereof, and optionally synthesizing compound(s) from said product data and further optionally comprising testing said synthesized compound(s) as or in a CRISPR-Cas9 system.

The testing can comprise analyzing the CRISPR-Cas9 system resulting from said synthesized selected structure(s), e.g., with respect to binding, or performing a desired function.

The output in the foregoing methods can comprise data transmission, e.g., transmission of information via telecommunication, telephone, video conference, mass communication, e.g., presentation such as a computer presentation (e.g. POWERPOINT), internet, email, documentary communication such as a computer program (e.g. WORD) document and the like. Accordingly, the invention also comprehends computer readable media containing: atomic co-ordinate data according to the herein-referenced Crystal Structure, said data defining the three dimensional structure of CRISPR-Cas9 or at least one sub-domain thereof, or structure factor data for CRISPR-Cas9, said structure factor data being derivable from the atomic co-ordinate data of herein-referenced Crystal Structure. The computer readable media can also contain any data of the foregoing methods. The invention further comprehends methods a computer system for generating or performing rational design as in the foregoing methods containing either: atomic co-ordinate data according to herein-referenced Crystal Structure, said data defining the three dimensional structure of CRISPR-Cas9 or at least one sub-domain thereof, or structure factor data for CRISPR-Cas9, said structure factor data being derivable from the atomic co-ordinate data of herein-referenced Crystal Structure. The invention further comprehends a method of doing business comprising providing to a user the computer system or the media or the three dimensional structure of CRISPR-Cas9 or at least one sub-domain thereof, or structure factor data for CRISPR-Cas9, said structure set forth in and said structure factor data being derivable from the atomic co-ordinate data of herein-referenced Crystal Structure, or the herein computer media or a herein data transmission.

A “binding site” or an “active site” comprises or consists essentially of or consists of a site (such as an atom, a functional group of an amino acid residue or a plurality of such atoms and/or groups) in a binding cavity or region, which may bind to a compound such as a nucleic acid molecule, which is/are involved in binding.

By “fitting”, is meant determining by automatic, or semi-automatic means, interactions between one or more atoms of a candidate molecule and at least one atom of a structure of the invention, and calculating the extent to which such interactions are stable. Interactions include attraction and repulsion, brought about by charge, steric considerations and the like. Various computer-based methods for fitting are described further

By “root mean square (or rms) deviation”, we mean the square root of the arithmetic mean of the squares of the deviations from the mean.

By a “computer system”, is meant the hardware means, software means and data storage means used to analyze atomic coordinate data. The minimum hardware means of the computer-based systems of the present invention typically comprises a central processing unit (CPU), input means, output means and data storage means. Desirably a display or monitor is provided to visualize structure data. The data storage means may be RAM or means for accessing computer readable media of the invention. Examples of such systems are computer and tablet devices running Unix, Windows or Apple operating systems.

By “computer readable media”, is meant any medium or media, which can be read and accessed directly or indirectly by a computer e.g., so that the media is suitable for use in the above-mentioned computer system. Such media include, but are not limited to: magnetic storage media such as floppy discs, hard disc storage medium and magnetic tape; optical storage media such as optical discs or CD-ROM; electrical storage media such as RAM and ROM; thumb drive devices; cloud storage devices and hybrids of these categories such as magnetic/optical storage media.

The invention comprehends the use of the protected guides described herein above in the optimized functional CRISPR-Cas enzyme systems described herein.

### Set Cover Approaches

In particular embodiments, a primer and/or probe is designed that can identify, for example, all viral and/or microbial species within a defined set of viruses and microbes. Such methods are described in certain example embodiments. A set cover solution may identify the minimal number of target sequence probes or primers needed to cover an entire target sequence or set of target sequences, e.g. a set of genomic sequences. Set cover approaches have been used previously to identify primers and/or microarray probes, typically in the 20 to 50 base pair range. See, e.g. Pearson et al., cs.virginia.edu/˜robins/papers/primers_dam11_final.pdf., Jabado et al. Nucleic Acids Res. 2006 34(22):6605-11, Jabado et al. Nucleic Acids Res. 2008, 36(1):e3 doi10.1093/nar/gkm1106, Duitama et al. Nucleic Acids Res. 2009, 37(8):2483-2492, Phillippy et al. BMC Bioinformatics. 2009, 10:293 doi:10.1186/1471-2105-10-293. Such approaches generally involved treating each primer/probe as k-mers and searching for exact matches or allowing for inexact matches using suffix arrays. In addition, the methods generally take a binary approach to detecting hybridization by selecting primers or probes such that each input sequence only needs to be bound by one primer or probe and the position of this binding along the sequence is irrelevant. Alternative methods may divide a target genome into pre-defined windows and effectively treat each window as a separate input sequence under the binary approach—i.e. they determine whether a given probe or guide RNA binds within each window and require that all of the windows be bound by the state of some primer or probe. Effectively, these approaches treat each element of the “universe” in the set cover problem as being either an entire input sequence or a pre-defined window of an input sequence, and each element is considered “covered” if the start of a probe or guide RNA binds within the element.

In some embodiments, the methods disclosed herein may be used to identify all variants of a given virus, or multiple different viruses in a single assay. Further, the method disclosed herein treat each element of the “universe” in the set cover problem as being a nucleotide of a target sequence, and each element is considered “covered” as long as a probe or guide RNA binds to some segment of a target genome that includes the element. Rather than only asking if a given primer or probe does or does not bind to a given window, such approaches may be used to detect a hybridization pattern—i.e. where a given primer or probe binds to a target sequence or target sequences—and then determines from those hybridization patterns the minimum number of primers or probes needed to cover the set of target sequences to a degree sufficient to enable both enrichment from a sample and sequencing of any and all target sequences. These hybridization patterns may be determined by defining certain parameters that minimize a loss function, thereby enabling identification of minimal probe or guide RNA sets in a way that allows parameters to vary for each species, e.g. to reflect the diversity of each species, as well as in a computationally efficient manner that cannot be achieved using a straightforward application of a set cover solution, such as those previously applied in the primer or probe design context.

The ability to detect multiple transcript abundances may allow for the generation of unique viral or microbial signatures indicative of a particular phenotype. Various machine learning techniques may be used to derive the gene signatures. Accordingly, the primers and/or probes of the invention may be used to identify and/or quantitate relative levels of biomarkers defined by the gene signature in order to detect certain phenotypes. In certain example embodiments, the gene signature indicates susceptibility to a particular treatment, resistance to a treatment, or a combination thereof.

In one aspect of the invention, a method comprises detecting one or more pathogens. In this manner, differentiation between infection of a subject by individual microbes may be obtained. In some embodiments, such differentiation may enable detection or diagnosis by a clinician of specific diseases, for example, different variants of a disease. Preferably the viral or pathogen sequence is a genome of the virus or pathogen or a fragment thereof. The method further may comprise determining the evolution of the pathogen. Determining the evolution of the pathogen may comprise identification of pathogen mutations, e.g. nucleotide deletion, nucleotide insertion, nucleotide substitution. Among the latter, there are non-synonymous, synonymous, and noncoding substitutions. Mutations are more frequently non-synonymous during an outbreak. The method may further comprise determining the substitution rate between two pathogen sequences analyzed as described above. Whether the mutations are deleterious or even adaptive would require functional analysis, however, the rate of non-synonymous mutations suggests that continued progression of this epidemic could afford an opportunity for pathogen adaptation, underscoring the need for rapid containment. Thus, the method may further comprise assessing the risk of viral adaptation, wherein the number non-synonymous mutations is determined. (Gire, et al., Science 345, 1369, 2014). The method may include diagnostic-guide-design as described elsewhere herein.

### RNA-Based Masking Construct

As used herein, a “masking construct” refers to a molecule that can be cleaved or otherwise deactivated by an activated CRISPR system effector protein described herein. The term “masking construct” may also be referred to in the alternative as a “detection construct.” In certain example embodiments, the masking construct is a RNA-based masking construct. The RNA-based masking construct comprises a RNA element that is cleavable by a CRISPR effector protein. Cleavage of the RNA element releases agents or produces conformational changes that allow a detectable signal to be produced. Example constructs demonstrating how the RNA element may be used to prevent or mask generation of detectable signal are described below and embodiments of the invention comprise variants of the same. Prior to cleavage, or when the masking construct is in an ‘active’ state, the masking construct blocks the generation or detection of a positive detectable signal. It will be understood that in certain example embodiments a minimal background signal may be produced in the presence of an active RNA masking construct. A positive detectable signal may be any signal that can be detected using optical, fluorescent, chemiluminescent, electrochemical or other detection methods known in the art. The term “positive detectable signal” is used to differentiate from other detectable signals that may be detectable in the presence of the masking construct. For example, in certain embodiments a first signal may be detected when the masking agent is present (i.e. a negative detectable signal), which then converts to a second signal (e.g. the positive detectable signal) upon detection of the target molecules and cleavage or deactivation of the masking agent by the activated CRISPR effector protein.

Accordingly, in certain embodiments of the invention, the RNA-based masking construct suppresses generation of a detectable positive signal or the RNA-based masking construct suppresses generation of a detectable positive signal by masking the detectable positive signal, or generating a detectable negative signal instead, or the RNA-based masking construct comprises a silencing RNA that suppresses generation of a gene product encoded by a reporting construct, wherein the gene product generates the detectable positive signal when expressed.

In further embodiments, the RNA-based masking construct is a ribozyme that generates the negative detectable signal, and wherein the positive detectable signal is generated when the ribozyme is deactivated, or the ribozyme converts a substrate to a first color and wherein the substrate converts to a second color when the ribozyme is deactivated.

In other embodiments, the RNA-based masking agent is an RNA aptamer, or the aptamer sequesters an enzyme, wherein the enzyme generates a detectable signal upon release from the aptamer by acting upon a substrate, or the aptamer sequesters a pair of agents that when released from the aptamers combine to generate a detectable signal.

In another embodiment, the RNA-based masking construct comprises an RNA oligonucleotide to which a detectable ligand and a masking component are attached. In another embodiment, the detectable ligand is a fluorophore and the masking component is a quencher molecule, or the reagents to amplify target RNA molecules such as, but not limited to, NASBA or RPA reagents.

In certain example embodiments, the masking construct may suppress generation of a gene product. The gene product may be encoded by a reporter construct that is added to the sample. The masking construct may be an interfering RNA involved in a RNA interference pathway, such as a short hairpin RNA (shRNA) or small interfering RNA (siRNA). The masking construct may also comprise microRNA (miRNA). While present, the masking construct suppresses expression of the gene product. The gene product may be a fluorescent protein or other RNA transcript or proteins that would otherwise be detectable by a labeled probe, aptamer, or antibody but for the presence of the masking construct. Upon activation of the effector protein the masking construct is cleaved or otherwise silenced allowing for expression and detection of the gene product as the positive detectable signal.

In certain example embodiments, the masking construct may sequester one or more reagents needed to generate a detectable positive signal such that release of the one or more reagents from the masking construct results in generation of the detectable positive signal. The one or more reagents may combine to produce a colorimetric signal, a chemiluminescent signal, a fluorescent signal, or any other detectable signal and may comprise any reagents known to be suitable for such purposes. In certain example embodiments, the one or more reagents are sequestered by RNA aptamers that bind the one or more reagents. The one or more reagents are released when the effector protein is activated upon detection of a target molecule and the RNA aptamers are degraded.

In certain example embodiments, the masking construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the masking construct are able to generate a detectable signal, for example by aggregation or simple increase in solution concentration. In certain example embodiments, the immobilized masking agent is a RNA-based aptamer that can be cleaved by the activated effector protein upon detection of a target molecule.

In certain other example embodiments, the masking construct binds to an immobilized reagent in solution thereby blocking the ability of the reagent to bind to a separate labeled binding partner that is free in solution. Thus, upon application of a washing step to a sample, the labeled binding partner can be washed out of the sample in the absence of a target molecule. However, if the effector protein is activated, the masking construct is cleaved to a degree sufficient to interfere with the ability of the masking construct to bind the reagent thereby allowing the labeled binding partner to bind to the immobilized reagent. Thus, the labeled binding partner remains after the wash step indicating the presence of the target molecule in the sample. In certain aspects, the masking construct that binds the immobilized reagent is an RNA aptamer. The immobilized reagent may be a protein and the labeled minding partner may be a labeled antibody. Alternatively, the immobilized reagent may be streptavidin and the labeled binding partner may be labeled biotin. The label on the binding partner used in the above embodiments may be any detectable label known in the art. In addition, other known binding partners may be used in accordance with the overall design described herein.

In certain example embodiments, the masking construct may comprise a ribozyme. Ribozymes are RNA molecules having catalytic properties. Ribozymes, both naturally and engineered, comprise or consist of RNA that may be targeted by the effector proteins disclosed herein. The ribozyme may be selected or engineered to catalyze a reaction that either generates a negative detectable signal or prevents generation of a positive control signal. Upon deactivation of the ribozyme by the activated effector protein the reaction generating a negative control signal, or preventing generation of a positive detectable signal, is removed thereby allowing a positive detectable signal to be generated. In one example embodiment, the ribozyme may catalyze a colorimetric reaction causing a solution to appear as a first color. When the ribozyme is deactivated the solution then turns to a second color, the second color being the detectable positive signal. An example of how ribozymes can be used to catalyze a colorimetric reaction are described in Zhao et al. “Signal amplification of glucosamine-6-phosphate based on ribozyme glmS,” Biosens Bioelectron. 2014; 16:337-42, and provide an example of how such a system could be modified to work in the context of the embodiments disclosed herein. Alternatively, ribozymes, when present can generate cleavage products of, for example, RNA transcripts. Thus, detection of a positive detectable signal may comprise detection of non-cleaved RNA transcripts that are only generated in the absence of the ribozyme.

In certain example embodiments, the one or more reagents is a protein, such as an enzyme, capable of facilitating generation of a detectable signal, such as a colorimetric, chemiluminescent, or fluorescent signal, that is inhibited or sequestered such that the protein cannot generate the detectable signal by the binding of one or more RNA aptamers to the protein. Upon activation of the effector proteins disclosed herein, the RNA aptamers are cleaved or degraded to an extent that they no longer inhibit the protein's ability to generate the detectable signal. In certain example embodiments, the aptamer is a thrombin inhibitor aptamer. In certain example embodiments the thrombin inhibitor aptamer has a sequence of GGGAACAAAGCUGAAGUACUUACCC (SEQ ID NO.4). When this aptamer is cleaved, thrombin will become active and will cleave a peptide colorimetric or fluorescent substrate. In certain example embodiments, the colorimetric substrate is para-nitroanilide (pNA) covalently linked to the peptide substrate for thrombin. Upon cleavage by thrombin, pNA is released and becomes yellow in color and easily visible to the eye. In certain example embodiments, the fluorescent substrate is 7-amino-4-methylcoumarin a blue fluorophore that can be detected using a fluorescence detector. Inhibitory aptamers may also be used for horseradish peroxidase (HRP), beta-galactosidase, or calf alkaline phosphatase (CAP) and within the general principals laid out above.

In certain embodiments, RNase activity is detected colorimetrically via cleavage of enzyme-inhibiting aptamers. One potential mode of converting RNase activity into a colorimetric signal is to couple the cleavage of an RNA aptamer with the re-activation of an enzyme that is capable of producing a colorimetric output. In the absence of RNA cleavage, the intact aptamer will bind to the enzyme target and inhibit its activity. The advantage of this readout system is that the enzyme provides an additional amplification step: once liberated from an aptamer via collateral activity (e.g. Cas13a collateral activity), the colorimetric enzyme will continue to produce colorimetric product, leading to a multiplication of signal.

In certain embodiments, an existing aptamer that inhibits an enzyme with a colorimetric readout is used. Several aptamer/enzyme pairs with colorimetric readouts exist, such as thrombin, protein C, neutrophil elastase, and subtilisin. These proteases have colorimetric substrates based upon pNA and are commercially available. In certain embodiments, a novel aptamer targeting a common colorimetric enzyme is used. Common and robust enzymes, such as beta-galactosidase, horseradish peroxidase, or calf intestinal alkaline phosphatase, could be targeted by engineered aptamers designed by selection strategies such as SELEX. Such strategies allow for quick selection of aptamers with nanomolar binding efficiencies and could be used for the development of additional enzyme/aptamer pairs for colorimetric readout.

In certain embodiments, RNase activity is detected colorimetrically via cleavage of RNA-tethered inhibitors. Many common colorimetric enzymes have competitive, reversible inhibitors: for example, beta-galactosidase can be inhibited by galactose. Many of these inhibitors are weak, but their effect can be increased by increases in local concentration. By linking local concentration of inhibitors to RNase activity, colorimetric enzyme and inhibitor pairs can be engineered into RNase sensors. The colorimetric RNase sensor based upon small-molecule inhibitors involves three components: the colorimetric enzyme, the inhibitor, and a bridging RNA that is covalently linked to both the inhibitor and enzyme, tethering the inhibitor to the enzyme. In the uncleaved configuration, the enzyme is inhibited by the increased local concentration of the small molecule; when the RNA is cleaved (e.g. by Cas13a collateral cleavage), the inhibitor will be released and the colorimetric enzyme will be activated.

In certain embodiments, RNase activity is detected colorimetrically via formation and/or activation of G-quadruplexes. G quadraplexes in DNA can complex with heme (iron (III)-protoporphyrin IX) to form a DNAzyme with peroxidase activity. When supplied with a peroxidase substrate (e.g. ABTS: (2,2′-Azinobis [3-ethylbenzothiazoline-6-sulfonic acid]-diammonium salt)), the G-quadraplex-heme complex in the presence of hydrogen peroxide causes oxidation of the substrate, which then forms a green color in solution. An example G-quadraplex forming DNA sequence is: GGGTAGGGCGGGTTGGGA (SEQ ID NO:5). By hybridizing an RNA sequence to this DNA aptamer, formation of the G-quadraplex structure will be limited. Upon RNase collateral activation (e.g. C2c2-complex collateral activation), the RNA staple will be cleaved allowing the G quadraplex to form and heme to bind. This strategy is particularly appealing because color formation is enzymatic, meaning there is additional amplification beyond RNase activation.

In certain example embodiments, the masking construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the masking construct are able to generate a detectable signal, for example by aggregation or simple increase in solution concentration. In certain example embodiments, the immobilized masking agent is a RNA-based aptamer that can be cleaved by the activated effector protein upon detection of a target molecule.

In one example embodiment, the masking construct comprises a detection agent that changes color depending on whether the detection agent is aggregated or dispersed in solution. For example, certain nanoparticles, such as colloidal gold, undergo a visible purple to red color shift as they move from aggregates to dispersed particles. Accordingly, in certain example embodiments, such detection agents may be held in aggregate by one or more bridge molecules. At least a portion of the bridge molecule comprises RNA. Upon activation of the effector proteins disclosed herein, the RNA portion of the bridge molecule is cleaved allowing the detection agent to disperse and resulting in the corresponding change in color. In certain example embodiments the, bridge molecule is a RNA molecule. In certain example embodiments, the detection agent is a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the Al³⁺, Ru³⁺, Zn²⁺, Fe³⁺, Ni²⁺ and Ca²⁺ ions.

When the RNA bridge is cut by the activated CRISPR effector, the beforementioned color shift is observed. In certain example embodiments the particles are colloidal metals. In certain other example embodiments, the colloidal metal is a colloidal gold. In certain example embodiments, the colloidal nanoparticles are 15 nm gold nanoparticles (AuNPs). Due to the unique surface properties of colloidal gold nanoparticles, maximal absorbance is observed at 520 nm when fully dispersed in solution and appear red in color to the naked eye. Upon aggregation of AuNPs, they exhibit a red-shift in maximal absorbance and appear darker in color, eventually precipitating from solution as a dark purple aggregate. In certain example embodiments the nanoparticles are modified to include DNA linkers extending from the surface of the nanoparticle. Individual particles are linked together by single-stranded RNA (ssRNA) bridges that hybridize on each end of the RNA to at least a portion of the DNA linkers. Thus, the nanoparticles will form a web of linked particles and aggregate, appearing as a dark precipitate. Upon activation of the CRISPR effectors disclosed herein, the ssRNA bridge will be cleaved, releasing the AU NPS from the linked mesh and producing a visible red color. Example DNA linkers and RNA bridge sequences are listed below. Thiol linkers on the end of the DNA linkers may be used for surface conjugation to the AuNPS. Other forms of conjugation may be used. In certain example embodiments, two populations of AuNPs may be generated, one for each DNA linker. This will help facilitate proper binding of the ssRNA bridge with proper orientation. In certain example embodiments, a first DNA linker is conjugated by the 3′ end while a second DNA linker is conjugated by the 5′ end.

| | |
|---|---|
| C2c2 colorimetric | TTATAACTATTCCTAAAAAAAAAAA/3ThioMC3-D/ |
| DNA1 | (SEQ. I.D. No: 6) |
| C2c2 colorimetric | /5ThioMC6- |
| DNA2 | D/AAAAAAAAAACTCCCCTAATAACAAT |
| | (SEQ. I.D. No. 7) |
| C2c2 colorimetric | GGGUAGGAAUAGUUAUAAUUUCCCUUUCCCAU |
| bridge | UGUUAUUAGGGAG (SEQ. I.D. No. 8) |

In certain other example embodiments, the masking construct may comprise an RNA oligonucleotide to which are attached a detectable label and a masking agent of that detectable label. An example of such a detectable label/masking agent pair is a fluorophore and a quencher of the fluorophore. Quenching of the fluorophore can occur as a result of the formation of a non-fluorescent complex between the fluorophore and another fluorophore or non-fluorescent molecule. This mechanism is known as ground-state complex formation, static quenching, or contact quenching. Accordingly, the RNA oligonucleotide may be designed so that the fluorophore and quencher are in sufficient proximity for contact quenching to occur. Fluorophores and their cognate quenchers are known in the art and can be selected for this purpose by one having ordinary skill in the art. The particular fluorophore/quencher pair is not critical in the context of this invention, only that selection of the fluorophore/quencher pairs ensures masking of the fluorophore. Upon activation of the effector proteins disclosed herein, the RNA oligonucleotide is cleaved thereby severing the proximity between the fluorophore and quencher needed to maintain the contact quenching effect. Accordingly, detection of the fluorophore may be used to determine the presence of a target molecule in a sample.

In certain other example embodiments, the masking construct may comprise one or more RNA oligonucleotides to which are attached one or more metal nanoparticles, such as gold nanoparticles. In some embodiments, the masking construct comprises a plurality of metal nanoparticles crosslinked by a plurality of RNA oligonucleotides forming a closed loop. In one embodiment, the masking construct comprises three gold nanoparticles crosslinked by three RNA oligonucleotides forming a closed loop. In some embodiments, the cleavage of the RNA oligonucleotides by the CRISPR effector protein leads to a detectable signal produced by the metal nanoparticles.

In certain other example embodiments, the masking construct may comprise one or more RNA oligonucleotides to which are attached one or more quantum dots. In some embodiments, the cleavage of the RNA oligonucleotides by the CRISPR effector protein leads to a detectable signal produced by the quantum dots.

In one example embodiment, the masking construct may comprise a quantum dot. The quantum dot may have multiple linker molecules attached to the surface. At least a portion of the linker molecule comprises RNA. The linker molecule is attached to the quantum dot at one end and to one or more quenchers along the length or at terminal ends of the linker such that the quenchers are maintained in sufficient proximity for quenching of the quantum dot to occur. The linker may be branched. As above, the quantum dot/quencher pair is not critical, only that selection of the quantum dot/quencher pair ensures masking of the fluorophore. Quantum dots and their cognate quenchers are known in the art and can be selected for this purpose by one having ordinary skill in the art Upon activation of the effector proteins disclosed herein, the RNA portion of the linker molecule is cleaved thereby eliminating the proximity between the quantum dot and one or more quenchers needed to maintain the quenching effect. In certain example embodiments the quantum dot is streptavidin conjugated. RNA are attached via biotin linkers and recruit quenching molecules with the sequences /5Biosg/UCUCGUACGUUC/3IAbRQSp/ (SEQ ID NO:9) or /5Biosg/UCUCGUACGUUCUCUCGUACGUUC/3IAbRQSp/ (SEQ ID NO:10), where /5Biosg/is a biotin tag and /3lAbRQSp/ is an Iowa black quencher. Upon cleavage, by the activated effectors disclosed herein the quantum dot will fluoresce visibly.

In a similar fashion, fluorescence energy transfer (FRET) may be used to generate a detectable positive signal. FRET is a non-radiative process by which a photon from an energetically excited fluorophore (i.e. “donor fluorophore”) raises the energy state of an electron in another molecule (i.e. “the acceptor”) to higher vibrational levels of the excited singlet state. The donor fluorophore returns to the ground state without emitting a fluoresce characteristic of that fluorophore. The acceptor can be another fluorophore or non-fluorescent molecule. If the acceptor is a fluorophore, the transferred energy is emitted as fluorescence characteristic of that fluorophore. If the acceptor is a non-fluorescent molecule the absorbed energy is loss as heat. Thus, in the context of the embodiments disclosed herein, the fluorophore/quencher pair is replaced with a donor fluorophore/acceptor pair attached to the oligonucleotide molecule. When intact, the masking construct generates a first signal (negative detectable signal) as detected by the fluorescence or heat emitted from the acceptor. Upon activation of the effector proteins disclosed herein the RNA oligonucleotide is cleaved and FRET is disrupted such that fluorescence of the donor fluorophore is now detected (positive detectable signal).

In certain example embodiments, the masking construct comprises the use of intercalating dyes which change their absorbance in response to cleavage of long RNAs to short nucleotides. Several such dyes exist. For example, pyronine-Y will complex with RNA and form a complex that has an absorbance at 572 nm. Cleavage of the RNA results in loss of absorbance and a color change. Methylene blue may be used in a similar fashion, with changes in absorbance at 688 nm upon RNA cleavage. Accordingly, in certain example embodiments the masking construct comprises an RNA and intercalating dye complex that changes absorbance upon the cleavage of RNA by the effector proteins disclosed herein.

In certain example embodiments, the masking construct may comprise an initiator for an HCR reaction. See e.g. Dirks and Pierce. PNAS 101, 15275-15728 (2004). HCR reactions utilize the potential energy in two hairpin species. When a single-stranded initiator having a portion of complementary to a corresponding region on one of the hairpins is released into the previously stable mixture, it opens a hairpin of one speces. This process, in turn, exposes a single-stranded region that opens a hairpin of the other species. This process, in turn, exposes a single stranded region identical to the original initiator. The resulting chain reaction may lead to the formation of a nicked double helix that grows until the hairpin supply is exhausted. Detection of the resulting products may be done on a gel or colorimetrically. Example colorimetric detection methods include, for example, those disclosed in Lu et al. “Ultra-sensitive colorimetric assay system based on the hybridization chain reaction-triggered enzyme cascade amplification ACS Appl Mater Interfaces, 2017, 9(1):167-175, Wang et al. “An enzyme-free colorimetric assay using hybridization chain reaction amplification and split aptamers” Analyst 2015, 150, 7657-7662, and Song et al. “Non covalent fluorescent labeling of hairpin DNA probe coupled with hybridization chain reaction for sensitive DNA detection.” Applied Spectroscopy, 70(4): 686-694 (2016).

In certain example embodiments, the masking construct may comprise a HCR initiator sequence and a cleavable structural element, such as a loop or hairpin, that prevents the initiator from initiating the HCR reaction. Upon cleavage of the structure element by an activated CRISPR effector protein, the initiator is then released to trigger the HCR reaction, detection thereof indicating the presence of one or more targets in the sample. In certain example embodiments, the masking construct comprises a hairpin with a RNA loop. When an activated CRISRP effector protein cuts the RNA loop, the initiator can be released to trigger the HCR reaction.

### Optical Barcodes, Barcodes, and Unique Molecular Identifier (UMI)

Systems as disclosed herein may comprise optical barcodes for one or more target molecules and an optical barcodes associated with the detection CRISPR system. For example, barcodes for one or more target molecules and a sample of interest comprising the target molecule can be merged with CRISPR detection system-containing droplets containing optical barcodes.

The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA such that multiple species can be sequenced together.

Barcoding may be performed based on any of the compositions or methods disclosed in patent publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell.

Optically encoded particles may be delivered to the discrete volumes randomly resulting in a random combination of optically encoded particles in each well, or a unique combination of optically encoded particles may be specifically assigned to each discrete volume. The observable combination of optically encoded particles may then be used to identify each discrete volume. Optical assessments, such as phenotype, may be made and recorded for each discrete volume. In some instances, the barcode may be an optically detectable barcode that can be visualized with light or fluorescence microscopy. In certain example embodiments, the optical barcode comprises a sub-set of fluorophores or quantum dots of distinguishable colors from a set of defined colors. In some instances, optically encoded particles may be delivered to the discrete volumes randomly resulting in a random combination of optically encoded particles in each well, or a unique combination of optically encoded particles may be specifically assigned to each discrete volume.

In an exemplary embodiment, 3 fluorescent dyes, e.g. Alexa Fluor 555, 594, 647, at different levels, 105 barcodes can be generated. The addition of a fourth dye can be used and can be extended to scale to hundreds of unique barcodes; similarly, five colors can increase the number of unique barcodes that may be achieved by varying the ratios of the colors. By labeling with distinct ratios of dyes, dye ratios can be chosen so that after normalization the dyes are evenly spaced in logarithmic coordinates.

In one embodiment, the assigned or random subset(s) of fluorophores received in each droplet or discrete volume dictates the observable pattern of discrete optically encoded particles in each discrete volume thereby allowing each discrete volume to be independently identified. Each discrete volume is imaged with the appropriate imaging technique to detect the optically encoded particles. For example, if the optically encoded particles are fluorescently labeled each discrete volume is imaged using a fluorescent microscope. In another example, if the optically encoded particles are colorimetrically labeled each discrete volume is imaged using a microscope having one or more filters that match the wave length or absorption spectrum or emission spectrum inherent to each color label. Other detection methods are contemplated that match the optical system used, e.g., those known in the art for detecting quantum dots, dyes, etc. The pattern of observed discrete optically encoded particles for each discrete volume may be recorded for later use.

Optical barcodes can optionally include a unique oligonucleotide sequence, method for generating can be as described in, for example, International Patent Application Publication No. WO/2014/047561 at [050]-[0115]. In one example embodiment, a primer particle identifier is incorporated in the target molecules. Next generation sequencing (NGS) techniques known in the art can be used for sequencing, with clustering by sequence similarity of the one or more target sequences. Alignment by sequence variation will allow for identification of optically encoded particles delivered to a discrete volume based on the particle identifiers incorporated in the aligned sequence information. In one embodiment, the particle identifier of each primer incorporated in the aligned sequence information indicates the pattern of optically encoded particles that is observable in the corresponding discrete volume from which the amplicons are generated. In this way the nucleic acid sequence variation can be correlated back to the originating discrete volume and further matched to the optical assessments, such as phenotype, made of the nucleic acid containing specimens in that discrete volume.

In preferred embodiments, sequencing is performed using unique molecular identifiers (UMI). The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single mRNA or target nucleic acid to be sequenced. The UMI may also be used to determine the number of transcripts that gave rise to an amplified product, or in the case of target barcodes as described herein, the number of binding events. In preferred embodiments, the amplification is by PCR or multiple displacement amplification (MDA).

In certain embodiments, an UMI with a random sequence of between 4 and 20 base pairs is added to a template, which is amplified and sequenced. In preferred embodiments, the UMI is added to the 5′ end of the template. Sequencing allows for high resolution reads, enabling accurate detection of true variants. As used herein, a “true variant” will be present in every amplified product originating from the original clone as identified by aligning all products with a UMI. Each clone amplified will have a different random UMI that will indicate that the amplified product originated from that clone. Background caused by the fidelity of the amplification process can be eliminated because true variants will be present in all amplified products and background representing random error will only be present in single amplification products (See e.g., Islam S. et al., 2014. Nature Methods No: 11, 163-166). Not being bound by a theory, the UMI's are designed such that assignment to the original can take place despite up to 4-7 errors during amplification or sequencing. Not being bound by a theory, an UMI may be used to discriminate between true barcode sequences.

Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

As disclosed herein, unique nucleic acid identifiers are used to label the target molecules and/or target nucleic acids, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example www.drmr.com/abcon). In certain embodiments, barcode tagging can occur via a barcode receiving adapter associate with (for example, attached to) a target molecule, as described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type NNNNNNNNNNNN (SEQ ID NO:11).

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites. A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. In some embodiments, the sequence of labeled target molecules is determined by non-sequencing based methods. For example, variable length probes or primers can be used to distinguish barcodes (for example, origin-specific barcodes) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool.

A nucleic acid barcode can be sequenced, for example, after cleavage, to determine the presence, quantity, or other feature of the target molecule. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an origin-specific barcode. The resultant nucleic acid barcode concatemer can be pooled with other such concatemers and sequenced. The sequencing reads can be used to identify which target molecules were originally present in which discrete volumes.

### Barcodes Reversibly Coupled to Solid Substrate

In some embodiments, the origin-specific barcodes are reversibly coupled to a solid or semisolid substrate. In some embodiments, the origin-specific barcodes further comprise a nucleic acid capture sequence that specifically binds to the target nucleic acids and/or a specific binding agent that specifically binds to the target molecules. In specific embodiments, the origin-specific barcodes include two or more populations of origin-specific barcodes, wherein a first population comprises the nucleic acid capture sequence and a second population comprises the specific binding agent that specifically binds to the target molecules. In some examples, the first population of origin-specific barcodes further comprises a target nucleic acid barcode, wherein the target nucleic acid barcode identifies the population as one that labels nucleic acids. In some examples, the second population of origin-specific barcodes further comprises a target molecule barcode, wherein the target molecule barcode identifies the population as one that labels target molecules.

Barcode with Cleavage Sites

A nucleic acid barcode may be cleavable from a specific binding agent, for example, after the specific binding agent has bound to a target molecule. In some embodiments, the origin-specific barcode further comprises one or more cleavage sites. In some examples, at least one cleavage site is oriented such that cleavage at that site releases the origin-specific barcode from a substrate, such as a bead, for example a hydrogel bead, to which it is coupled. In some examples, at least one cleavage site is oriented such that the cleavage at the site releases the origin-specific barcode from the target molecule specific binding agent. In some examples, a cleavage site is an enzymatic cleavage site, such an endonuclease site present in a specific nucleic acid sequence. In other embodiments, a cleavage site is a peptide cleavage site, such that a particular enzyme can cleave the amino acid sequence. In still other embodiments, a cleavage site is a site of chemical cleavage.

### Barcode Adapters

In some embodiments, the target molecule is attached to an origin-specific barcode receiving adapter, such as a nucleic acid. In some examples, the origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In some embodiments, the barcode receiving adapter may be associated with (for example, attached to) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/barcode concatemer.

Barcode with Capture Moiety

In some embodiments, an origin-specific barcode further includes a capture moiety, covalently or non-covalently linked. Thus, in some embodiments the origin-specific barcode, and anything bound or attached thereto, that include a capture moiety are captured with a specific binding agent that specifically binds the capture moiety. In some embodiments, the capture moiety is adsorbed or otherwise captured on a surface. In specific embodiments, a targeting probe is labeled with biotin, for instance by incorporation of biotin-16-UTP during in vitro transcription, allowing later capture by streptavidin. Other means for labeling, capturing, and detecting an origin-specific barcode include: incorporation of aminoallyl-labeled nucleotides, incorporation of sulfhydryl-labeled nucleotides, incorporation of allyl- or azide-containing nucleotides, and many other methods described in Bioconjugate Techniques (2^{nd} Ed), Greg T. Hermanson, Elsevier (2008), which is specifically incorporated herein by reference. In some embodiments, the targeting probes are covalently coupled to a solid support or other capture device prior to contacting the sample, using methods such as incorporation of aminoallyl-labeled nucleotides followed by 1-Ethyl-3-(3-dimethylaminopropyl)carbodiimide (EDC) coupling to a carboxy-activated solid support, or other methods described in Bioconjugate Techniques. In some embodiments, the specific binding agent has been immobilized for example on a solid support, thereby isolating the origin-specific barcode.

### Other Barcoding Embodiments

DNA barcoding is also a taxonomic method that uses a short genetic marker in an organism's DNA to identify it as belonging to a particular species. It differs from molecular phylogeny in that the main goal is not to determine classification but to identify an unknown sample in terms of a known classification. Kress et al., “Use of DNA barcodes to identify flowering plants” Proc. Natl. Acad. Sci. U.S.A. 102(23):8369-8374 (2005). Barcodes are sometimes used in an effort to identify unknown species or assess whether species should be combined or separated. Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009). Barcoding has been used, for example, for identifying plant leaves even when flowers or fruit are not available, identifying the diet of an animal based on stomach contents or feces, and/or identifying products in commerce (for example, herbal supplements or wood). Soininen et al., “Analysing diet of small herbivores: the efficiency of DNA barcoding coupled with high-throughput pyrosequencing for deciphering the composition of complex plant mixtures” Frontiers in Zoology 6:16 (2009).

It has been suggested that a desirable locus for DNA barcoding should be standardized so that large databases of sequences for that locus can be developed. Most of the taxa of interest have loci that are sequencable without species-specific PCR primers. CBOL Plant Working Group, “A DNA barcode for land plants” PNAS 106(31):12794-12797 (2009). Further, these putative barcode loci are believed short enough to be easily sequenced with current technology. Kress et al., “DNA barcodes: Genes, genomics, and bioinformatics” PNAS 105(8):2761-2762 (2008). Consequently, these loci would provide a large variation between species in combination with a relatively small amount of variation within a species. Lahaye et al., “DNA barcoding the floras of biodiversity hotspots” Proc Natl Acad Sci USA 105(8):2923-2928 (2008).

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31):12569 (2009).

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94).

Target molecules, as described herein can include any target nucleic acid sequence, that, in embodiments, the one or more guide RNAs are designed to bind to one or more target molecules that are diagnostic for a disease state. In further embodiments, the disease state is an infection, an organ disease, a blood disease, an immune system disease, a cancer, a brain and nervous system disease, an endocrine disease, a pregnancy or childbirth-related disease, an inherited disease, or an environmentally-acquired disease. In still further embodiments, the disease state is an infection, including a microbial infection.

In further embodiments, the infection is caused by a virus, a bacterium, or a fungus, or the infection is a viral infection. In specific embodiments, the viral infection is caused by a double-stranded RNA virus, a positive sense RNA virus, a negative sense RNA virus, a retrovirus, or a combination thereof. In certain embodiments, the application can achieve multiplexed strain discrimination. In some embodiments, pathogen subtyping can be detected, in one embodiment, influenza subtyping, Staph or strep subtyping, and bacterial superinfection subtype detection can be performed. In one preferred embodiment, multiplexed detection and identification of all H and N subtypes of Influenza A virus can be performed. In one aspect, pooled (or arrayed) crRNAs are used to capture variation within subtypes. In certain instances, the infection is HIV. In an embodiment, drug resistant mutations in HIV Reverse Transcriptase can be performed via SNP detection. In some embodiments, the mutation can be K65R, K103N, V106M, Y181C, M184V, G190A. Similarly, SNP detection in other infections can be performed, such as in tuberculosis. In some embodiments, the mutation may be katG, 315ACC: Isoniazid resistance, rpoB, 531TTG: Rifampin resistance, gyrA, 94GGC: Fluoroquinolone resistance, rrs, 1401G: Aminoglycoside resistance. Additionally, HIV/TB co-infections can be detected. Massive multiplexing to detect pan-viral, viral zone pan-viral, pan-bacterial or pan-pathogen detection can be achieved.

As described herein, a sample containing target molecules for use with the invention may be a biological or environmental sample, such as a food sample (fresh fruits or vegetables, meats), a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

The biological sample may be further processed prior to further evaluation, including, for example by enriching or isolating cells of interest. In one aspect, cells in a biological sample have been first enriched or sorted prior to further processing and/or library preparation. In embodiments, the cells are sorted by fluorescence-activated cell sorting (FACS) or magnetic-activated cell sorting (MACS). In an example embodiment, cells are first sorted using, for example, antibody coated (para)magnetic beads to sort antigen-specific T cells. Both tube-based and column-based methods for MACS can be used to isolate rare cell populations, or to further enrich a cell (sub)population of interest. Multiple rounds of MACS can further enrich cells, with successive rounds enriching with the same epitope tag or with different epitope tags. See, e.g. Lee et al., J. Biomol. Tech. 2012 Jull 23(2): 69-77. Cells can be eluted removing the magnetic bead where necessary, and further processed, including further enrichment. In one embodiment, T cells can be isolated from peripheral blood lymphocytes by lysing the red blood cells and depleting the monocytes, for example, by centrifugation through a PERCOLL™ gradient. A specific subpopulation of T cells, such as CD28+, T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. Once cells of interest are sorted, enriched, and/or isolated, the samples can be further processed, for example, by extraction of nucleic acids, appending of barcodes, droplet formation and analysis.

In some embodiments, the biological sample may include, but is not necessarily limited to, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface. In specific embodiments, the sample may be blood, plasma or serum obtained from a human patient.

In some embodiments, the sample may be a plant sample. In some embodiments, the sample may be a crude sample. In some embodiments, the sample may be a purified sample.

### Microfluidic Devices Comprising an Array of Microwells

Microfluidic devices comprise an array of microwells with at least one flow channel beneath the microwells. In certain example embodiments, the device is a microfluidic device that generates and/or merges different droplets (i.e. individual discrete volumes). For example, a first set of droplets may be formed containing samples to be screened and a second set of droplets formed containing the elements of the systems described herein. The first and second set of droplets are then merged and then diagnostic methods as described herein are carried out on the merged droplet set.

Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

An example of microfluidic device that may be used in the context of the invention is described in Kulesa, et al. PNAS, 115, 6685-6690, incorporated herein by reference.

In certain example embodiments, the device may comprise individual wells, such as microplate wells. The size of the microplate wells may be the size of standard 6, 24, 96, 384, 1536, 3456, or 9600 sized wells. In certain embodiments, the microwells can number at more than 40,0000 or more than 190,000. In certain example embodiments, the elements of the systems described herein may be freeze dried and applied to the surface of the well prior to distribution and use.

Microwell chips can be designed as disclosed in Attorney Docket No. 52199-505P03US or in U.S. patent application Ser. No. 15/559,381 incorporated herein by reference. In one embodiment, the microwell chip can be designed in a format measuring around 6.2×7.2 cm, containing 49200 microwells, or a larger format, measuring 7.4×10 cm, containing 97, 194 microwells. The array of microwells can be shaped, for example, as two circles of a diameter of about 50-300 μm, in particular embodiments at 150 μm diameter set at 10% overlap. The array of microwells can be arranged in a hexagonal lattice at 50 μm inter-well spacing. In some instances, the microwells can be arranged in other shapes, spacing and sizes in order to hold a varying number of droplets. The microwell chips are advantageously, in some embodiments, sized for use with standard laboratory equipment, including imaging equipment such as microscopes.

In an exemplary method, compounds can be mixed with a unique ratio of fluorescent dyes (e.g. Alexa Fluor 555, 594, 647). Each mixture of target molecule with a dye mixture can be emulsified into droplets. Similarly, each detection CRISPR system with optical barcode can be emulsified into droplets. In some embodiments, the droplets are approximately 1 nL each. The CRISPR detection system droplets and target molecule droplets can then be combined and applied to the microwell chip. The droplets can be combined by simple mixing or other methods of combination. In one exemplary embodiment, the microwell chip is suspended on a platform such as a hydrophobic glass slide with removable spacers that can be clamped from above and below by clamps or other securing means, which can be, for example, neodymium magnets. The gap between the chip and the glass created by the spacers can be loaded with oil, and the pool of droplets injected into the chip, continuing to flow the droplets by injecting more oil and draining excess droplets. After loading is completed, the chip can be washed with oil, and spacers can be removed to seal microwells against the glass slide and clamp closed. The chip can be imaged, for example with an epifluorescence microscope, droplets merged to mix the compounds in each microwell by applying an AC electric field, for example, supplied by a corona treater, and subsequently treated according to desired protocols. In one embodiment, the microwell can be incubated at 37° C. with measurement of fluorescence using epifluoresecnce microscope. Following manipulation of the droplets, the droplets can be eluted off of the microwell as described herein for additional analyses, processing and/or manipulations.

The devices disclosed may further comprise inlet and outlet ports, or openings, which in turn may be connected to valves, tubes, channels, chambers, and syringes and/or pumps for the introduction and extraction of fluids into and from the device. The devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In certain example embodiments, the devices are connected to controllers with programmable valves that work together to move fluids through the device. In certain example embodiments, the devices are connected to the controllers discussed in further detail below. The devices may be connected to flow actuators, controllers, and sample loading devices by tubing that terminates in metal pins for insertion into inlet ports on the device.

The present invention may be used with a wireless lab-on-chip (LOC) diagnostic sensor system (see e.g., U.S. Pat. No. 9,470,699 “Diagnostic radio frequency identification sensors and applications thereof”). In certain embodiments, the present invention is performed in a LOC controlled by a wireless device (e.g., a cell phone, a personal digital assistant (PDA), a tablet) and results are reported to said device.

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

In preferred embodiments, the LOC may be a microfluidic device. The LOC may be a passive chip, wherein the chip is powered and controlled through a wireless device. In certain embodiments, the LOC includes a microfluidic channel for holding reagents and a channel for introducing a sample. In certain embodiments, a signal from the wireless device delivers power to the LOC and activates mixing of the sample and assay reagents. Specifically, in the case of the present invention, the system may include a masking agent, CRISPR effector protein, and guide RNAs specific for a target molecule. Upon activation of the LOC, the microfluidic device may mix the sample and assay reagents. Upon mixing, a sensor detects a signal and transmits the results to the wireless device. In certain embodiments, the unmasking agent is a conductive RNA molecule. The conductive RNA molecule may be attached to the conductive material. Conductive molecules can be conductive nanoparticles, conductive proteins, metal particles that are attached to the protein or latex or other beads that are conductive. In certain embodiments, if DNA or RNA is used then the conductive molecules can be attached directly to the matching DNA or RNA strands. The release of the conductive molecules may be detected across a sensor. The assay may be a one step process.

Since the electrical conductivity of the surface area can be measured precisely quantitative results are possible on the disposable wireless RFID electro-assays. Furthermore, the test area can be very small allowing for more tests to be done in a given area and therefore resulting in cost savings. In certain embodiments, separate sensors each associated with a different CRISPR effector protein and guide RNA immobilized to a sensor are used to detect multiple target molecules. Not being bound by a theory, activation of different sensors may be distinguished by the wireless device.

In addition to the conductive methods described herein, other methods may be used that rely on RFID or Bluetooth as the basic low-cost communication and power platform for a disposable RFID assay. For example, optical means may be used to assess the presence and level of a given target molecule. In certain embodiments, an optical sensor detects unmasking of a fluorescent masking agent.

In certain embodiments, the device of the present invention may include handheld portable devices for diagnostic reading of an assay (see e.g., Vashist et al., Commercial Smartphone-Based Devices and Smart Applications for Personalized Healthcare Monitoring and Management, Diagnostics 2014, 4(3), 104-128; mReader from Mobile Assay; and Holomic Rapid Diagnostic Test Reader).

As noted herein, certain embodiments allow detection via colorimetric change which has certain attendant benefits when embodiments are utilized in POC situations and or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present invention is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, in certain embodiments utilizing quantum dot-based masking constructs, use of a hand-held UV light, or other suitable device, may be successfully used to detect a signal owing to the near complete quantum yield provided by quantum dots.

### Individual Discrete Volumes

In some embodiments, the CRISPR system is contained in individual discrete volumes, each individual discrete volume comprising a CRISPR effector protein, one or more guide RNAs designed to bind to corresponding target molecule, and an RNA-based masking construct. In some instances, each of these individual discrete volumes are droplets. In a particularly preferred embodiment, the droplets are provided as a first set of droplets, each droplet containing a CRISPR system. In some embodiments, the target molecule, or sample, is contained in individual discrete volumes, each individual discrete volume comprising a target molecule. In some instances, each of these individual discrete volumes are droplets. In a particularly preferred embodiment, the droplets are provided as a second set of droplets, each droplet containing a target molecule.

In one aspect, the embodiments disclosed herein can include a first set of droplets directed to a nucleic acid detection system comprising a CRISPR system, one or more guide RNAs designed to bind to corresponding target molecules, a masking construct, and optional amplification reagents to amplify target nucleic acid molecules in a sample. In certain example embodiments, the system may further comprise one or more detection aptamers. The one or more detection aptamers may comprise an RNA polymerase site or primer binding site. The one or more detection aptamers specifically bind one or more target polypeptides and are configured such that the RNA polymerase site or primer binding site is exposed only upon binding of the detection aptamer to a target peptide. Exposure of the RNA polymerase site facilitates generation of a trigger RNA oligonucleotide using the aptamer sequence as a template. Accordingly, in such embodiments the one or more guide RNAs are configured to bind to a trigger RNA.

An “individual discrete volume” is a discrete volume or discrete space, such as a container, receptacle, or other defined volume or space that can be defined by properties that prevent and/or inhibit migration of nucleic acids, CRISPR detection systems, and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. In particularly preferred embodiments, the individual discrete volumes are droplets. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable is that some reagents, such as buffers, chemical activators, or other agents maybe passed in or through the discrete volume, while other material, such as target molecules, maybe maintained in the discrete volume or space. As explained herein, a droplet system allows for the separation of compounds until initiation of a reaction is desired. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are droplets.

### Droplets

The droplets as provided herein are typically water-in-oil microemulsions formed with an oil input channel and an aqueous input channel. The droplets can be formed by a variety of dispersion methods known in the art. In one particular embodiment, a large number of uniform droplets in oil phase can be made by microemulsion. Exemplary methods can include, for example, R-junction geometry where an aqueous phase is sheared by oil and thereby generates droplets; flow-focusing geometry where droplets are produced by shearing the aqueous stream from two directions; or co-flow geometry where an aqueous phase is ejected through a thin capillary, placed coaxially inside a bigger capillary through which oil is pumped.

The use of monodisperse aqueous droplets can be generated by a microfluidic device as a water-in-oil emulsion. In one embodiment, the droplets are carried in a flowing oil phase and stabilized by a surfactant. In one aspect single cells or single organelles or single molecules (proteins, RNA, DNA) are encapsulated into uniform droplets from an aqueous solution/dispersion. In a related aspect, multiple cells or multiple molecules may take the place of single cells or single molecules.

The aqueous droplets of volume ranging from 1 pL to 10 nL work as individual reactors. 10⁴ to 10⁵ single cells in droplets may be processed and analyzed in a single run. To utilize microdroplets for rapid large-scale chemical screening or complex biological library identification, different species of microdroplets, each containing the specific chemical compounds or biological probes cells or molecular barcodes of interest, have to be generated and combined at the preferred conditions, e.g., mixing ratio, concentration, and order of combination. Each species of droplet is introduced at a confluence point in a main microfluidic channel from separate inlet microfluidic channels. Preferably, droplet volumes are chosen by design such that one species is larger than others and moves at a different speed, usually slower than the other species, in the carrier fluid, as disclosed in U.S. Publication No. US 2007/0195127 and International Publication No. WO 2007/089541, each of which are incorporated herein by reference in their entirety. The channel width and length is selected such that faster species of droplets catch up to the slowest species. Size constraints of the channel prevent the faster moving droplets from passing the slower moving droplets resulting in a train of droplets entering a merge zone. Multi-step chemical reactions, biochemical reactions, or assay detection chemistries often require a fixed reaction time before species of different type are added to a reaction. Multi-step reactions are achieved by repeating the process multiple times with a second, third or more confluence points each with a separate merge point. Highly efficient and precise reactions and analysis of reactions are achieved when the frequencies of droplets from the inlet channels are matched to an optimized ratio and the volumes of the species are matched to provide optimized reaction conditions in the combined droplets. Fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc. In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled.

In one arrangement, the expansion and/or contraction of liquid reservoirs may be used to steer or sort a fluidic droplet into a channel, e.g., by causing directed movement of the liquid containing the fluidic droplet. In another, the expansion and/or contraction of the liquid reservoir may be combined with other flow-controlling devices and methods, e.g., as described herein. Non-limiting examples of devices able to cause the expansion and/or contraction of a liquid reservoir include pistons. Key elements for using microfluidic channels to process droplets include: (1) producing droplet of the correct volume, (2) producing droplets at the correct frequency and (3) bringing together a first stream of sample droplets with a second stream of sample droplets in such a way that the frequency of the first stream of sample droplets matches the frequency of the second stream of sample droplets. Preferably, bringing together a stream of sample droplets with a stream of premade library droplets in such a way that the frequency of the library droplets matches the frequency of the sample droplets. Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. It is desirable for one of the species introduced at the confluence to be a pre-made library of droplets where the library contains a plurality of reaction conditions, e.g., a library may contain plurality of different compounds at a range of concentrations encapsulated as separate library elements for screening their effect on cells or enzymes, alternatively a library could be composed of a plurality of different primer pairs encapsulated as different library elements for targeted amplification of a collection of loci, alternatively a library could contain a plurality of different antibody species encapsulated as different library elements to perform a plurality of binding assays. The introduction of a library of reaction conditions onto a substrate is achieved by pushing a premade collection of library droplets out of a vial with a drive fluid. The drive fluid is a continuous fluid. The drive fluid may comprise the same substance as the carrier fluid (e.g., a fluorocarbon oil). For example, if a library consists of ten pico-liter droplets is driven into an inlet channel on a microfluidic substrate with a drive fluid at a rate of 10,000 pico-liters per second, then nominally the frequency at which the droplets are expected to enter the confluence point is 1000 per second. However, in practice droplets pack with oil between them that slowly drains. Over time the carrier fluid drains from the library droplets and the number density of the droplets (number/mL) increases. Hence, a simple fixed rate of infusion for the drive fluid does not provide a uniform rate of introduction of the droplets into the microfluidic channel in the substrate. Moreover, library-to-library variations in the mean library droplet volume result in a shift in the frequency of droplet introduction at the confluence point. Thus, the lack of uniformity of droplets that results from sample variation and oil drainage provides another problem to be solved. For example if the nominal droplet volume is expected to be 10 pico-liters in the library, but varies from 9 to 11 pico-liters from library-to-library then a 10,000 pico-liter/second infusion rate will nominally produce a range in frequencies from 900 to 1,100 droplet per second. In short, sample to sample variation in the composition of dispersed phase for droplets made on chip, a tendency for the number density of library droplets to increase over time and library-to-library variations in mean droplet volume severely limit the extent to which frequencies of droplets may be reliably matched at a confluence by simply using fixed infusion rates. In addition, these limitations also have an impact on the extent to which volumes may be reproducibly combined. Combined with typical variations in pump flow rate precision and variations in channel dimensions, systems are severely limited without a means to compensate on a run-to-run basis. The foregoing facts not only illustrate a problem to be solved, but also demonstrate a need for a method of instantaneous regulation of microfluidic control over microdroplets within a microfluidic channel.

Combinations of surfactant(s) and oils must be developed to facilitate generation, storage, and manipulation of droplets to maintain the unique chemical/biochemical/biological environment within each droplet of a diverse library. Therefore, the surfactant and oil combination should (1) stabilize droplets against uncontrolled coalescence during the drop forming process and subsequent collection and storage, (2) minimize transport of any droplet contents to the oil phase and/or between droplets, and (3) maintain chemical and biological inertness with contents of each droplet (e.g., no adsorption or reaction of encapsulated contents at the oil-water interface, and no adverse effects on biological or chemical constituents in the droplets). In addition to the requirements on the droplet library function and stability, the surfactant-in-oil solution must be coupled with the fluid physics and materials associated with the platform. Specifically, the oil solution must not swell, dissolve, or degrade the materials used to construct the microfluidic chip, and the physical properties of the oil (e.g., viscosity, boiling point, etc.) must be suited for the flow and operating conditions of the platform. Droplets formed in oil without surfactant are not stable to permit coalescence, so surfactants must be dissolved in the oil that is used as the continuous phase for the emulsion library. Surfactant molecules are amphiphilic--part of the molecule is oil soluble, and part of the molecule is water soluble. When a water-oil interface is formed at the nozzle of a microfluidic chip for example in the inlet module described herein, surfactant molecules that are dissolved in the oil phase adsorb to the interface. The hydrophilic portion of the molecule resides inside the droplet and the fluorophilic portion of the molecule decorates the exterior of the droplet. The surface tension of a droplet is reduced when the interface is populated with surfactant, so the stability of an emulsion is improved. In addition to stabilizing the droplets against coalescence, the surfactant should be inert to the contents of each droplet and the surfactant should not promote transport of encapsulated components to the oil or other droplets. A droplet library may be made up of a number of library elements that are pooled together in a single collection (see, e.g., US Patent Publication No. 2010002241).

Libraries may vary in complexity from a single library element to 10¹⁵ library elements or more. Each library element may be one or more given components at a fixed concentration. The element may be, but is not limited to, cells, organelles, virus, bacteria, yeast, beads, amino acids, proteins, polypeptides, nucleic acids, polynucleotides or small molecule chemical compounds. The element may contain an identifier such as a label. The terms “droplet library” or “droplet libraries” are also referred to herein as an “emulsion library” or “emulsion libraries.” These terms are used interchangeably throughout the specification. A cell library element may include, but is not limited to, hybridomas, B-cells, primary cells, cultured cell lines, cancer cells, stem cells, cells obtained from tissue, or any other cell type. Cellular library elements are prepared by encapsulating a number of cells from one to hundreds of thousands in individual droplets. The number of cells encapsulated is usually given by Poisson statistics from the number density of cells and volume of the droplet. However, in some cases the number deviates from Poisson statistics as described in Edd et al., “Controlled encapsulation of single-cells into monodisperse picolitre drops.” Lab Chip, 8(8): 1262-1264, 2008. The discrete nature of cells allows for libraries to be prepared in mass with a plurality of cellular variants all present in a single starting media and then that media is broken up into individual droplet capsules that contain at most one cell. These individual droplets capsules are then combined or pooled to form a library consisting of unique library elements. Cell division subsequent to, or in some embodiments following, encapsulation produces a clonal library element.

In certain embodiments, a bead based library element may contain one or more beads, of a given type and may also contain other reagents, such as antibodies, enzymes or other proteins. In the case where all library elements contain different types of beads, but the same surrounding media, the library elements may all be prepared from a single starting fluid or have a variety of starting fluids. In the case of cellular libraries prepared in mass from a collection of variants, such as genomically modified, yeast or bacteria cells, the library elements will be prepared from a variety of starting fluids. Often it is desirable to have exactly one cell per droplet with only a few droplets containing more than one cell when starting with a plurality of cells or yeast or bacteria, engineered to produce variants on a protein. In some cases, variations from Poisson statistics may be achieved to provide an enhanced loading of droplets such that there are more droplets with exactly one cell per droplet and few exceptions of empty droplets or droplets containing more than one cell. Examples of droplet libraries are collections of droplets that have different contents, ranging from beads, cells, small molecules, DNA, primers, antibodies. Smaller droplets may be in the order of femtoliter (fL) volume drops, which are especially contemplated with the droplet dispensors. The volume may range from about 5 to about 600 fL. The larger droplets range in size from roughly 0.5 micron to 500 micron in diameter, which corresponds to about 1 pico liter to 1 nano liter. However, droplets may be as small as 5 microns and as large as 500 microns. Preferably, the droplets are at less than 100 microns, about 1 micron to about 100 microns in diameter. The most preferred size is about 20 to 40 microns in diameter (10 to 100 picoliters). The preferred properties examined of droplet libraries include osmotic pressure balance, uniform size, and size ranges. The droplets within the emulsion libraries of the present invention may be contained within an immiscible oil which may comprise at least one fluorosurfactant. In some embodiments, the fluorosurfactant within the immiscible fluorocarbon oil may be a block copolymer consisting of one or more perfluorinated polyether (PFPE) blocks and one or more polyethylene glycol (PEG) blocks. In other embodiments, the fluorosurfactant is a triblock copolymer consisting of a PEG center block covalently bound to two PFPE blocks by amide linking groups. The presence of the fluorosurfactant (similar to uniform size of the droplets in the library) is critical to maintain the stability and integrity of the droplets and is also essential for the subsequent use of the droplets within the library for the various biological and chemical assays described herein. Fluids (e.g., aqueous fluids, immiscible oils, etc.) and other surfactants that may be utilized in the droplet libraries of the present invention are described in greater detail herein.

The present invention can accordingly involve an emulsion library which may comprise a plurality of aqueous droplets within an immiscible oil (e.g., fluorocarbon oil) which may comprise at least one fluorosurfactant, wherein each droplet is uniform in size and may comprise the same aqueous fluid and may comprise a different library element. The present invention also provides a method for forming the emulsion library which may comprise providing a single aqueous fluid which may comprise different library elements, encapsulating each library element into an aqueous droplet within an immiscible fluorocarbon oil which may comprise at least one fluorosurfactant, wherein each droplet is uniform in size and may comprise the same aqueous fluid and may comprise a different library element, and pooling the aqueous droplets within an immiscible fluorocarbon oil which may comprise at least one fluorosurfactant, thereby forming an emulsion library. For example, in one type of emulsion library, all different types of elements (e.g., cells or beads), may be pooled in a single source contained in the same medium. After the initial pooling, the cells or beads are then encapsulated in droplets to generate a library of droplets wherein each droplet with a different type of bead or cell is a different library element. The dilution of the initial solution enables the encapsulation process. In some embodiments, the droplets formed will either contain a single cell or bead or will not contain anything, i.e., be empty. In other embodiments, the droplets formed will contain multiple copies of a library element. The cells or beads being encapsulated are generally variants on the same type of cell or bead. In another example, the emulsion library may comprise a plurality of aqueous droplets within an immiscible fluorocarbon oil, wherein a single molecule may be encapsulated, such that there is a single molecule contained within a droplet for every 20-60 droplets produced (e.g., 20, 25, 30, 35, 40, 45, 50, 55, 60 droplets, or any integer in between). Single molecules may be encapsulated by diluting the solution containing the molecules to such a low concentration that the encapsulation of single molecules is enabled. Formation of these libraries may rely on limiting dilutions.

The present invention also provides an emulsion library which may comprise at least a first aqueous droplet and at least a second aqueous droplet within an oil, in one embodiment a fluorocarbon oil, which may comprise at least one surfactant, in one embodiment a fluorosurfactant, wherein the at least first and the at least second droplets are uniform in size and comprise a different aqueous fluid and a different library element. The present invention also provides a method for forming the emulsion library which may comprise providing at least a first aqueous fluid which may comprise at least a first library of elements, providing at least a second aqueous fluid which may comprise at least a second library of elements, encapsulating each element of said at least first library into at least a first aqueous droplet within an immiscible fluorocarbon oil which may comprise at least one fluorosurfactant, encapsulating each element of said at least second library into at least a second aqueous droplet within an immiscible fluorocarbon oil which may comprise at least one fluorosurfactant, wherein the at least first and the at least second droplets are uniform in size and may comprise a different aqueous fluid and a different library element, and pooling the at least first aqueous droplet and the at least second aqueous droplet within an immiscible fluorocarbon oil which may comprise at least one fluorosurfactant thereby forming an emulsion library.

One of skill in the art will recognize that methods and systems of the invention need not be limited to any particular type of sample, and methods and systems of the invention may be used with any type of organic, inorganic, or biological molecule (see, e.g, US Patent Publication No. 20120122714).

In particular embodiments the sample may include nucleic acid target molecules. Nucleic acid molecules may be synthetic or derived from naturally occurring sources. In one embodiment, nucleic acid molecules may be isolated from a biological sample containing a variety of other components, such as proteins, lipids and non-template nucleic acids. Nucleic acid target molecules may be obtained from any cellular material, obtained from an animal, plant, bacterium, fungus, or any other cellular organism. In certain embodiments, the nucleic acid target molecules may be obtained from a single cell. Biological samples for use in the present invention may include viral particles or preparations. Nucleic acid target molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid target molecules may also be isolated from cultured cells, such as a primary cell culture or a cell line. The cells or tissues from which target nucleic acids are obtained may be infected with a virus or other intracellular pathogen. A sample may also be total RNA extracted from a biological specimen, a cDNA library, viral, or genomic DNA. Generally, nucleic acid may be extracted from a biological sample by a variety of techniques such as those described by Maniatis, et al., Molecular Cloning: A Laboratory Manual, Cold Spring Harbor, N.Y., pp. 280-281 (1982). Nucleic acid molecules may be single-stranded, double-stranded, or double-stranded with single-stranded regions (for example, stem- and loop-structures). Nucleic acid obtained from biological samples typically may be fragmented to produce suitable fragments for analysis. Target nucleic acids may be fragmented or sheared to desired length, using a variety of mechanical, chemical and/or enzymatic methods. DNA may be randomly sheared via sonication, e.g. Covaris method, brief exposure to a DNase, or using a mixture of one or more restriction enzymes, or a transposase or nicking enzyme. RNA may be fragmented by brief exposure to an RNase, heat plus magnesium, or by shearing. The RNA may be converted to cDNA. If fragmentation is employed, the RNA may be converted to cDNA before or after fragmentation. In one embodiment, nucleic acid from a biological sample is fragmented by sonication. In another embodiment, nucleic acid is fragmented by a hydroshear instrument. Generally, individual nucleic acid target molecules may be from about 40 bases to about 40 kb. Nucleic acid molecules may be single-stranded, double-stranded, or double-stranded with single-stranded regions (for example, stem- and loop-structures). A biological sample as described herein may be homogenized or fractionated in the presence of a detergent or surfactant. The concentration of the detergent in the buffer may be about 0.05% to about 10.0%. The concentration of the detergent may be up to an amount where the detergent remains soluble in the solution. In one embodiment, the concentration of the detergent is between 0.1% to about 2%. The detergent, particularly a mild one that is nondenaturing, may act to solubilize the sample. Detergents may be ionic or nonionic. Examples of nonionic detergents include triton, such as the Triton™ X series (Triton™ X-100 t-Oct-C6H4--(OCH2--CH2)xOH, x=9-10, Triton™ X-100R, Triton™ X-114 x=7-8), octyl glucoside, polyoxyethylene(9)dodecyl ether, digitonin, IGEPAL™ CA630 octylphenyl polyethylene glycol, n-octyl-beta-D-glucopyranoside (betaOG), n-dodecyl-beta, Tween™. 20 polyethylene glycol sorbitan monolaurate, Tween™ 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14E06), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant. Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid. Size selection of the nucleic acids may be performed to remove very short fragments or very long fragments. The nucleic acid fragments may be partitioned into fractions which may comprise a desired number of fragments using any suitable method known in the art. Suitable methods to limit the fragment size in each fragment are known in the art. In various embodiments of the invention, the fragment size is limited to between about 10 and about 100 Kb or longer. A sample in or as to the instant invention may include individual target proteins, protein complexes, proteins with translational modifications, and protein/nucleic acid complexes. Protein targets include peptides, and also include enzymes, hormones, structural components such as viral capsid proteins, and antibodies. Protein targets may be synthetic or derived from naturally-occurring sources. The invention protein targets may be isolated from biological samples containing a variety of other components including lipids, non-template nucleic acids, and nucleic acids. Protein targets may be obtained from an animal, bacterium, fungus, cellular organism, and single cells. Protein targets may be obtained directly from an organism or from a biological sample obtained from the organism, including bodily fluids such as blood, urine, cerebrospinal fluid, seminal fluid, saliva, sputum, stool and tissue. Protein targets may also be obtained from cell and tissue lysates and biochemical fractions. An individual protein is an isolated polypeptide chain. A protein complex includes two or polypeptide chains. Samples may include proteins with post translational modifications including but not limited to phosphorylation, methionine oxidation, deamidation, glycosylation, ubiquitination, carbamylation, s-carboxymethylation, acetylation, and methylation. Protein/nucleic acid complexes include cross-linked or stable protein-nucleic acid complexes. Extraction or isolation of individual proteins, protein complexes, proteins with translational modifications, and protein/nucleic acid complexes is performed using methods known in the art.

The invention can thus involve forming sample droplets. The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc. The content of each of which is incorporated by reference herein in its entirety. The present invention may relate to systems and methods for manipulating droplets within a high-throughput microfluidic system. A microfluid droplet may encapsulate a differentiated cell, the cell is lysed and its mRNA is hybridized onto a capture bead containing barcoded oligo dT primers on the surface, all inside the droplet. The barcode is covalently attached to the capture bead via a flexible multi-atom linker like PEG. In a preferred embodiment, the droplets are broken by addition of a fluorosurfactant (like perfluorooctanol), washed, and collected. A reverse transcription (RT) reaction is then performed to convert each cell's mRNA into a first strand cDNA that is both uniquely barcoded and covalently linked to the mRNA capture bead. Subsequently, a universal primer via a template switching reaction is amended using conventional library preparation protocols to prepare an RNA-Seq library. Since all of the mRNA from any given cell is uniquely barcoded, a single library is sequenced and then computationally resolved to determine which mRNAs came from which cells. In this way, through a single sequencing run, tens of thousands (or more) of distinguishable transcriptomes can be simultaneously obtained. The oligonucleotide sequence may be generated on the bead surface. During these cycles, beads were removed from the synthesis column, pooled, and aliquoted into four equal portions by mass; these bead aliquots were then placed in a separate synthesis column and reacted with either dG, dC, dT, or dA phosphoramidite. In other instances, dinucleotide, trinucleotides, or oligonucleotides that are greater in length are used, in other instances, the oligo-dT tail is replaced by gene specific oligonucleotides to prime specific targets (singular or plural), random sequences of any length for the capture of all or specific RNAs. This process was repeated 12 times for a total of 4¹²=16,777,216 unique barcode sequences. Upon completion of these cycles, 8 cycles of degenerate oligonucleotide synthesis were performed on all the beads, followed by 30 cycles of dT addition. In other embodiments, the degenerate synthesis is omitted, shortened (less than 8 cycles), or extended (more than 8 cycles); in others, the 30 cycles of dT addition are replaced with gene specific primers (single target or many targets) or a degenerate sequence. The aforementioned microfluidic system is regarded as the reagent delivery system microfluidic library printer or droplet library printing system of the present invention. Droplets are formed as sample fluid flows from droplet generator which contains lysis reagent and barcodes through microfluidic outlet channel which contains oil, towards junction. Defined volumes of loaded reagent emulsion, corresponding to defined numbers of droplets, are dispensed on-demand into the flow stream of carrier fluid. The sample fluid may typically comprise an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible with nucleic acid molecules can be used. The carrier fluid may include one that is immiscible with the sample fluid. The carrier fluid can be a non-polar solvent, decane (e.g., tetradecane or hexadecane), fluorocarbon oil, silicone oil, an inert oil such as hydrocarbon, or another oil (for example, mineral oil). The carrier fluid may contain one or more additives, such as agents which reduce surface tensions (surfactants). Surfactants can include Tween, Span, fluorosurfactants, and other agents that are soluble in oil relative to water. In some applications, performance is improved by adding a second surfactant to the sample fluid. Surfactants can aid in controlling or optimizing droplet size, flow and uniformity, for example by reducing the shear force needed to extrude or inject droplets into an intersecting channel. This can affect droplet volume and periodicity, or the rate or frequency at which droplets break off into an intersecting channel. Furthermore, the surfactant can serve to stabilize aqueous emulsions in fluorinated oils from coalescing. Droplets may be surrounded by a surfactant which stabilizes the droplets by reducing the surface tension at the aqueous oil interface. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglyceryl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates). In some cases, an apparatus for creating a single-cell sequencing library via a microfluidic system provides for volume-driven flow, wherein constant volumes are injected over time. The pressure in fluidic cannels is a function of injection rate and channel dimensions. In one embodiment, the device provides an oil/surfactant inlet; an inlet for an analyte; a filter, an inlet for mRNA capture microbeads and lysis reagent; a carrier fluid channel which connects the inlets; a resistor; a constriction for droplet pinch-off, a mixer; and an outlet for drops. In an embodiment the invention provides apparatus for creating a single-cell sequencing library via a microfluidic system, which may comprise: an oil-surfactant inlet which may comprise a filter and a carrier fluid channel, wherein said carrier fluid channel may further comprise a resistor; an inlet for an analyte which may comprise a filter and a carrier fluid channel, wherein said carrier fluid channel may further comprise a resistor; an inlet for mRNA capture microbeads and lysis reagent which may comprise a filter and a carrier fluid channel, wherein said carrier fluid channel further may comprise a resistor; said carrier fluid channels have a carrier fluid flowing therein at an adjustable or predetermined flow rate; wherein each said carrier fluid channels merge at a junction; and said junction being connected to a mixer, which contains an outlet for drops. Accordingly, an apparatus for creating a single-cell sequencing library via a microfluidic system icrofluidic flow scheme for single-cell RNA-seq is envisioned. Two channels, one carrying cell suspensions, and the other carrying uniquely barcoded mRNA capture bead, lysis buffer and library preparation reagents meet at a junction and is immediately co-encapsulated in an inert carrier oil, at the rate of one cell and one bead per drop. In each drop, using the bead's barcode tagged oligonucleotides as cDNA template, each mRNA is tagged with a unique, cell-specific identifier. The invention also encompasses use of a Drop-Seq library of a mixture of mouse and human cells. The carrier fluid may be caused to flow through the outlet channel so that the surfactant in the carrier fluid coats the channel walls. The fluorosurfactant can be prepared by reacting the perfluorinated polyether DuPont Krytox 157 FSL, FSM, or FSH with aqueous ammonium hydroxide in a volatile fluorinated solvent. The solvent and residual water and ammonia can be removed with a rotary evaporator. The surfactant can then be dissolved (e.g., 2.5 wt %) in a fluorinated oil (e.g., Fluorinert (3M)), which then serves as the carrier fluid. Activation of sample fluid reservoirs to produce regent droplets is based on the concept of dynamic reagent delivery (e.g., combinatorial barcoding) via an on-demand capability. The on-demand feature may be provided by one of a variety of technical capabilities for releasing delivery droplets to a primary droplet, as described herein.

From this disclosure and herein cited documents and knowledge in the art, it is within the ambit of the skilled person to develop flow rates, channel lengths, and channel geometries; and establish droplets containing random or specified reagent combinations can be generated on demand and merged with the “reaction chamber” droplets containing the samples/cells/substrates of interest. By incorporating a plurality of unique tags into the additional droplets and joining the tags to a solid support designed to be specific to the primary droplet, the conditions that the primary droplet is exposed to may be encoded and recorded. For example, nucleic acid tags can be sequentially ligated to create a sequence reflecting conditions and order of same. Alternatively, the tags can be added independently appended to solid support. Non-limiting examples of a dynamic labeling system that may be used to bioinformatically record information can be found at US Provisional Patent Application entitled “Compositions and Methods for Unique Labeling of Agents” filed Sep. 21, 2012 and Nov. 29, 2012. In this way, two or more droplets may be exposed to a variety of different conditions, where each time a droplet is exposed to a condition, a nucleic acid encoding the condition is added to the droplet each ligated together or to a unique solid support associated with the droplet such that, even if the droplets with different histories are later combined, the conditions of each of the droplets are remain available through the different nucleic acids. Non-limiting examples of methods to evaluate response to exposure to a plurality of conditions can be found at US Provisional patent application filed Sep. 21, 2012, and U.S. patent application Ser. No. 15/303,874 filed Apr. 17, 2015 entitled “Systems and Methods for Droplet Tagging.” Accordingly, in or as to the invention it is envisioned that there can be the dynamic generation of molecular barcodes (e.g., DNA oligonucleotides, fluorophores, etc.) either independent from or in concert with the controlled delivery of various compounds of interest (siRNA, CRISPR guide RNAs, reagents, etc.). For example, unique molecular barcodes can be created in one array of nozzles while individual compounds or combinations of compounds can be generated by another nozzle array. Barcodes/compounds of interest can then be merged with CRISPR detection system-containing droplets. An electronic record in the form of a computer log file can be kept to associate the barcode delivered with the downstream reagent(s) delivered. This methodology makes it possible to efficiently screen a large population of samples according to the methods disclosed herein. The device and techniques of the disclosed invention facilitate efforts to perform studies that require data resolution at the single cell (or single molecule) level and in a cost-effective manner. A high-throughput and high-resolution delivery of reagents to individual emulsion droplets that may contain samples of target molecules for further evaluation through the use of monodisperse aqueous droplets that are generated one by one in a microfluidic chip as a water-in-oil emulsion.

### Detection of Proteins

The systems, devices, and methods disclosed herein may also be adapted for detection of polypeptides (or other molecules) in addition to detection of nucleic acids, via incorporation of a specifically configured polypeptide detection aptamer. The polypeptide detection aptamers are distinct from the masking construct aptamers discussed above. First, the aptamers are designed to specifically bind to one or more target molecules. In one example embodiment, the target molecule is a target polypeptide. In another example embodiment, the target molecule is a target chemical compound, such as a target therapeutic molecule. Methods for designing and selecting aptamers with specificity for a given target, such as SELEX, are known in the art. In addition to specificity to a given target the aptamers are further designed to incorporate a RNA polymerase promoter binding site. In certain example embodiments, the RNA polymerase promoter is a T7 promoter. Prior to binding the apatamer binding to a target, the RNA polymerase site is not accessible or otherwise recognizable to a RNA polymerase. However, the aptamer is configured so that upon binding of a target the structure of the aptamer undergoes a conformational change such that the RNA polymerase promoter is then exposed. An aptamer sequence downstream of the RNA polymerase promoter acts as a template for generation of a trigger RNA oligonucleotide by a RNA polymerase. Thus, the template portion of the aptamer may further incorporate a barcode or other identifying sequence that identifies a given aptamer and its target. Guide RNAs as described above may then be designed to recognize these specific trigger oligonucleotide sequences. Binding of the guide RNAs to the trigger oligonucleotides activates the CRISPR effector proteins which proceeds to deactivate the masking constructs and generate a positive detectable signal as described herein.

Accordingly, in certain example embodiments, the methods disclosed herein comprise the additional step of distributing a sample or set of sample into a set of individual discrete volumes, each individual discrete volume comprising peptide detection aptamers, a CRISPR effector protein, one or more guide RNAs, a masking construct, and incubating the sample or set of samples under conditions sufficient to allow binding of the detection aptamers to the one or more target molecules, wherein binding of the aptamer to a corresponding target results in exposure of the RNA polymerase promoter binding site such that synthesis of a trigger RNA is initiated by the binding of a RNA polymerase to the RNA polymerase promoter binding site.

In another example embodiment, binding of the aptamer may expose a primer binding site upon binding of the aptamer to a target polypeptide. For example, the aptamer may expose a RPA primer binding site. Thus, the addition or inclusion of the primer will then feed into an amplification reaction, such as the RPA reaction outlined above.

In certain example embodiments, the aptamer may be a conformation-switching aptamer, which upon binding to the target of interest may change secondary structure and expose new regions of single-stranded DNA. In certain example embodiments, these new-regions of single-stranded DNA may be used as substrates for ligation, extending the aptamers and creating longer ssDNA molecules which can be specifically detected using the embodiments disclosed herein. The aptamer design could be further combined with ternary complexes for detection of low-epitope targets, such as glucose (Yang et al. 2015: pubs.acs.org/doi/abs/10.1021/acs.analchem.5b01634). Example conformation shifting aptamers and corresponding guide RNAs (crRNAs) are shown below.

| | |
|---|---|
| Thrombin aptamer | (SEQ. ID NO: 12) |
| Thrombin ligation probe | (SEQ. ID NO: 13) |
| Thrombin RPA forward 1 | (SEQ. ID NO: 14) |
| primer | |
| Thrombin RPA forward 2 | (SEQ. ID NO: 15) |
| primer | |
| Thrombin RPA reverse 1 | (SEQ. ID NO: 16) |
| primer | |
| Thrombin crRNA 1 | (SEQ. ID NO: 17) |
| Thrombin crRNA 2 | (SEQ. ID NO: 18) |
| Thrombin crRNA 3 | (SEQ. ID NO: 19) |
| PTK7 full length amplicon | (SEQ. ID NO: 20) |
| control | |
| PTK7 aptamer | (SEQ. ID NO: 21) |
| PTK7 ligation probe | (SEQ. ID NO: 22) |
| PTK7 RPA forward 1 primer | (SEQ. ID NO: 23) |
| PTK7 RPA reverse 1 primer | (SEQ. ID NO: 24) |
| PTK7 crRNA 1 | (SEQ. ID NO: 25) |
| PTK7 crRNA 2 | (SEQ. ID NO: 26) |
| PTK7 crRNA 3 | (SEQ. ID NO: 27) |

### Amplification

In certain example embodiments, target RNAs and/or DNAs may be amplified prior to activating the CRISPR effector protein. In some instances, amplification is performed prior to formation of a droplet set comprising the target molecule. Other embodiments permit amplification to be performed subsequent to formation of a droplet set comprising the target molecule, and, accordingly, may include nucleic acid amplification reagents in the droplet comprising the target molecule. Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM). In some preferred embodiments, the RNA or DNA amplification is RPA or PCR.

In certain example embodiments, the RNA or DNA amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. Binding of the target RNAs by the guide RNAs then leads to activation of the CRISPR effector protein and the methods proceed as outlined above. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. The amplified target RNA can then in turn be detected by the CRISPR effector system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH₄)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents. Amplification of nucleic acids may be performed using specific thermal cycle machinery or equipment, and may be performed in single reactions or in bulk, such that any desired number of reactions may be performed simultaneously. In some instances, amplification can be performed in droplets or prior to droplet formation. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In some instances, the nucleic acid amplification reagents comprise recombinase polymerase amplification (RPA) reagents, nucleic acid sequence-based amplification (NASBA) reagents, loop-mediated isothermal amplification (LAMP) reagents, strand displacement amplification (SDA) reagents, helicase-dependent amplification (HDA) reagents, nicking enzyme amplification reaction (NEAR) reagents, RT-PCR reagents, multiple displacement amplification (MDA) reagents, rolling circle amplification (RCA) reagents, ligase chain reaction (LCR) reagents, ramification amplification method (RAM) reagents, transposase based amplification reagents; or Programmable CRISPR Nicking Amplification (PCNA) reagents. In certain embodiments, detection of DNA with the methods or systems of the invention requires transcription of the (amplified) DNA into RNA prior to detection.

It will be evident that detection methods of the invention can involve nucleic acid amplification and detection procedures in various combinations. The nucleic acid to be detected can be any naturally occurring or synthetic nucleic acid, including but not limited to DNA and RNA, which may be amplified by any suitable method to provide an intermediate product that can be detected. Detection of the intermediate product can be by any suitable method including but not limited to binding and activation of a Cas protein which produces a detectable signal moiety by direct or collateral activity.

Amplification and/or Enhancement of Detectable Positive Signal

In certain example embodiments, further modification may be introduced that further amplify the detectable positive signal. For example, activated CRISPR effector protein collateral activation may be use to generate a secondary target or additional guide sequence, or both. In one example embodiment, the reaction solution would contain a secondary target that is spiked in at high concentration. The secondary target may be distinct from the primary target (i.e. the target for which the assay is designed to detect) and in certain instances may be common across all reaction volumes. A secondary guide sequence for the secondary target may be protected, e.g. by a secondary structural feature such as a hairpin with a RNA loop, and unable to bind the second target or the CRISPR effector protein. Cleavage of the protecting group by an activated CRISPR effector protein (i.e. after activation by formation of complex with the primary target(s) in solution) and formation of a complex with free CRISPR effector protein in solution and activation from the spiked in secondary target. In certain other example embodiments, a similar concept is used with a second guide sequence to a secondary target sequence. The secondary target sequence may be protected a structural feature or protecting group on the secondary target. Cleavage of a protecting group off the secondary target then allows additional CRISPR effector protein/second guide sequence/secondary target complex to form. In yet another example embodiment, activation of CRISPR effector protein by the primary target(s) may be used to cleave a protected or circularized primer, which is then released to perform an isothermal amplification reaction, such as those disclosed herein, on a template that encodes a secondary guide sequence, secondary target sequence, or both. Subsequent transcription of this amplified template would produce more secondary guide sequence and/or secondary target sequence, followed by additional CRISPR effector protein collateral activation.

### Methods

In an aspect, the embodiments disclosed herein are directed to methods for detecting target nucleic acids in a sample using the systems described herein. The methods disclosed herein can, in some embodiments, comprise the steps of generating a first set of droplets, each droplet in the first set of droplets comprising at least one target molecule and an optical barcode; generating a second set of droplets, each droplet in the second set of droplets comprising a detection CRISPR system comprising an RNA targeting effector protein and one or more guide RNAs designed to bind to corresponding target molecules, an masking construct and optionally an optical barcode. The first and second set of droplets are typically combined into a pool of droplets by mixing or agitating the first and second set of droplets. The pool of droplets can then be flooded onto a microfluidic device comprising an array of microwells and at least one flow channel beneath the microwells, the microwells sized to capture at least two droplets; detecting the optical barcodes of the droplets captured in each microwell; merging the droplets captured in each microwell to formed merged droplets in each microwell, at least a subset of the merged droplets comprising a detection CRISPR system and a target sequence; initiating the detection reaction; and measuring a detectable signal of each merged droplet at one or more time periods.

### Generation of Droplets

Regarding generation of a first set of droplets, in one aspect generating a first set of droplets, each first droplet containing a detection CRISPR system, the detection CRISPR system can comprise an RNA targeting effector protein and one or more guide RNAs designed to bind to corresponding target molecules, an RNA-based masking construct and an optical barcode as described herein. In particular embodiments the step of generating a second set of droplets each droplet in the second set of droplets comprises at least one target molecule and an optional optical barcode as provided herein.

Subsequent to generation of a first set of droplets and a second set of droplets, the first set and second set of droplets are combined into a pool of droplets. The combining can be effected by any means to combine the first and second sets. In one exemplary embodiment, the sets of droplets are mixed to combine into a pool of droplets.

Once a pool of droplets is generated, the step of flowing the pool of droplets is performed. The flowing of the pool of droplets is performed by loading the droplets onto a microfluidic device containing a plurality of microwells. The microwells are sized to capture at least two droplets. Optionally, subsequent to loading, surfactant is washed out.

Once the droplets are loaded into the microwell array, a step of detecting the optical barcode of the droplets captured in each microwell is performed. In some instances, the detecting the optical barcode is performed by low magnification fluorescence scan when the optical barcodes are fluorescence barcodes. Regardless of the type of optical barcode, the barcodes for each droplet are unique, and thus the content of each droplet can be identified. The manner of detection will be selected according to the type of optical barcode utilized. The droplets contained in each microwell are then merged. Merging can be performed by applying an electrical field. At least a subset of the merged droplets comprise a detection CRISPR system and a target sequence.

After merging of the droplets, the detection reaction is then initiated. In some embodiments, initiating the detection reaction comprises incubating the merged droplets. Subsequent to the detection reaction, the merged droplets are subjected to an optical assay, which in some instances is a low magnification fluorescence scan to generate an assay score.

In some embodiments, the methods can comprise a step of amplifying target molecules. Amplification of the target molecules can be performed prior to or subsequent to the generation of the first set of droplets.

In yet another aspect, the embodiments disclosed herein are directed to a method for detecting polypeptides. The method for detecting polypeptides is similar to the method for detecting target nucleic acids described above. However, a peptide detection aptamer is also included. The peptide detection aptamers function as described above and facilitate generation of a trigger oligonucleotide upon binding to a target polypeptide. The guide RNAs are designed to recognize the trigger oligonucleotides thereby activating the CRISPR effector protein. Deactivation of the masking construct by the activated CRISPR effector protein leads to unmasking, release, or generation of a detectable positive signal.

Multiplexed detection diagnostics utilizing a reporter construct (e.g. fluorescence protein) can rapidly detect target sequences, diagnose drug resistance SNPs, and discriminate between strains and subtypes of microbial species. In the case of evaluating a sample for the presence of one or more strains of a microbial species, for example, a set of target molecules from a sample are evaluated utilizing a set of CRISPR systems contained in a second set of droplets, each CRISPR system containing different guide RNAs. After combination of the first and second set of droplets, the combinations are tested rapidly and in replicates. Each target molecule to be tested is placed in a microplate well. Mono-disperse droplets comprising the target molecule to be screened can be formed using an aqueous and an oil input channel. The target molecule droplets are then loaded onto a microfluidic device. Each target molecule is labeled with a barcode. When two or more droplets are merged, the combined optical barcodes identify which target molecule and/or CRISPR system are present in the merged droplet. The barcode is an optically detectable barcode visualized with light or fluorescence microscopy or an oligonucleotide barcode that is detected off-chip.

As described herein, samples containing target molecules to which the guide RNAs are targeted, are loaded into one set of droplets and merged with droplet(s) comprising the guide RNAs and CRISPR system. Reporter systems incorporated in the CRISPR system droplets express an optically detectable marker (e.g. fluorescent protein) in the masking construct. The set of droplets including a CRISPR system comprising an effector protein and one or more guide RNAs designed to bind to corresponding target molecules, and an RNA-based masking construct. After the droplets are merged, the identity of the molecular species in each well can be determined by optically scanning each microwell to read the optical barcode. Optical measurement of the reporter system can occur simultaneously with optical scanning of the barcode. Thus, simultaneous gathering of experimental data and molecular species identification is possible with use of this combinatorial screening system.

In some cases, the microfluidic device is incubated for a period of time prior to imaging and imaged at multiple time points to track changes in the measured amount of reporter over time. Additionally, for some experiments, merged droplets are eluted off of the microfluidic device for off-chip evaluation (see e.g., International Publication No. WO2016/149661, hereby incorporated by reference in its entirety for all purposes, elution is particularly discussed at [0056]-[0059]). With the disclosed processing strategy, parallel handling of millions of droplets reaches the scale needed for combinatorial screening. Additionally, the droplets' nanoliter volume reduces compound consumption required for screening. The present disclosure incorporates optical barcodes and parallel manipulation of droplets in large fixed-position spatial arrays to link droplet identity with assay results. A unique advantage of the present system is the parsimonious use of the compounds screened in the 2 nL assay volumes. The platform herein leverages the high-throughput potential of droplet microfluidic systems, and substitutes the deterministic liquid handling operations needed to construct combination of pairs of compounds with parallel merging of random pairs of droplets in a microwell device. Unique advantages of this method are that it can be hand-operated at high-throughput, and that assay miniaturization in microwells enables use of small sample volumes. When combined with SHEROCK technology, the methods provide a powerful detection technology that can be massively multiplexed utilizing smaller sample sizes.

The techniques herein provide a processing platform that tests all pairwise combinations of a set of input compounds in three steps. First, target molecules are combined with a color barcode (unique ratios of two, three, four or more fluorescent dyes). The target molecules may be barcoded by their ratio of fluorescent dyes (e.g. red, green, blue, and the like). Subsequent to sample processing, the target molecules are then emulsified into water in oil droplets, preferably of a size of about 1 nanoliter. In some embodiments, a surfactant can be included to stabilize the droplets. Standard multi-channel micropipette techniques may be used to combine the droplets into one pool. A second set of droplets are prepared containing CRISPR systems, an optional optical barcode using a ratio of fluorescent dyes, and an RNA masking compound. The first set and second set of droplets are mixed into one large pool, with the droplets subsequently loaded into a microwell array such that each microwell captures two droplets at random. In some embodiments, the microwell array after loading is then sealed to a glass substrate to limit microwell cross-contamination and evaporation. In some instances, the microwell array is fixed to an assembly by mechanical clamping. The contents of each droplet are encoded by fluorescence barcodes resulting from unique ratios of two, three, four or more fluorescent dyes pre-mixed with the first set and second set of droplets identified.

A low-magnification (2-4×) epifluorescence microscope can be used to identify the contents of each droplet and/or well. The two droplets in each well are then merged, applying a high voltage AC electric field to induce droplet merging. Subsequent to merging, SHERLOCK reactions are initiated, with samples incubated in some embodiments at 37° C. Subsequently, the array is imaged to determine an optical phenotype (e.g. positive fluorescence) and map this measurement to the pair of compounds previously identified in each well. Microwell array designs limiting compound exchange after loading are particularly preferred, one exemplary way is to mechanically seal the microwell array subsequent to the loading of the droplets.

In one aspect, the embodiments described herein are directed to methods for multiplex screening of nucleic acid sequence variations in one or more nucleic acid containing specimens. The nucleic acid sequence variations may include natural sequence variability, variations in gene expression, engineered genetic perturbations, or a combination thereof. The nucleic acid containing specimen may be cellular or acellular. The nucleic acid containing specimens are prepared as droplets containing an optical barcode. A second set of droplets containing a CRISPR detection system and an optical barcode is prepared. In some instances, the barcode may be an optically detectable barcode that can be visualized with light or fluorescence microscopy. In certain example embodiments, the optical barcode comprises a sub-set of fluorophores or quantum dots of distinguishable colors from a set of defined colors. In some instances, optically encoded particles may be delivered to the discrete volumes randomly resulting in a random combination of optically encoded particles in each well, or a unique combination of optically encoded particles may be specifically assigned to each discrete volume. Random distribution of the optically encoded particles may be achieved by pumping, mixing, rocking, or agitation of the assay platform for a time sufficient to allow for distribution to all discrete volumes. One of ordinary skill in the art can select the appropriate mechanism for randomly distributing the optically encoded particles across discrete volumes based on the assay platform used.

The observable combination of optically encoded particles may then be used to identify each discrete volume. Optical assessments, such as phenotype, may be made and recorded for each discrete volume, for example, with a fluorescent microscope or other imaging device. As shown in FIG. 13, using 3 fluorescent dyes, e.g. Alexa Fluor 555, 594, 647, at different levels, 105 barcodes can be generated. The addition of a fourth dye can be used and can be extended to scale to hundreds of unique barcodes; similarly, five colors can increase the number of unique barcodes that may be achieved by varying the ratios of the colors.

For example, nucleic acid-functionalized particles can be synthesized onto a solid support and subsequently labeled with distinct ratios of dyes, for example, FAM, Cy3 and Cy5, or 3 fluorescent dyes, e.g. Alexa Fluor 555, 594, 647, at different levels, 105 barcodes can be generated.

In one embodiment, the assigned or random subset(s) of fluorophores received in each droplet or discrete volume dictates the observable pattern of discrete optically encoded particles in each discrete volume thereby allowing each discrete volume to be independently identified. Each discrete volume is imaged with the appropriate imaging technique to detect the optically encoded particles. For example, if the optically encoded particles are fluorescently labeled each discrete volume is imaged using a fluorescent microscope. In another example, if the optically encoded particles are colorimetrically labeled each discrete volume is imaged using a microscope having one or more filters that match the wave length or absorption spectrum or emission spectrum inherent to each color label. Other detection methods are contemplated that match the optical system used, e.g., those known in the art for detecting quantum dots, dyes, etc. The pattern of observed discrete optically encoded particles for each discrete volume may be recorded for later use.

In addition, optical assessments can be made subsequent to merging of the droplets, and incubation of the CRISPR detection system with the target molecules. Once the target molecule is detected by a guide molecule, the CRISPR effector protein is activated, deactivating the masking construct, for example, by cleaving the masking construct such that a detectable positive signal is unmasked, released, or generated. Detection and measuring a detectable signal of each merged droplet at one or more time periods can be performed, indicating the presence of target molecules when, for example the positive detectable signal is present.

Further embodiments of the invention are described in the following numbered paragraphs.

1. A method for detecting target molecules comprising:

generating a first set of droplets, each droplet in the first set of droplets comprising a detection CRISPR system comprising a Cas protein and one or more guide RNAs designed to bind to corresponding target molecules, an masking construct and an optical barcode;

generating a second set of droplets, each droplet in the second set of droplets comprising at least one target molecule and optionally an optical barcode;

combining the first set and second set of droplets into a pool of droplets and flowing the pool of droplets onto a microfluidic device comprising an array of microwells and at least one flow channel beneath the microwells, the microwells sized to capture at least two droplets;

detecting the optical barcodes of the droplets captured in each microwell;

merging the droplets captured in each microwell to formed merged droplets in each microwell, at least a subset of the merged droplets comprising a detection CRISPR system and a target sequence;

initiating the detection reaction; and

measuring a detectable signal of each merged droplet at one or more time periods, optionally continuously.

2. The method according to paragraph 1, further comprising a step of amplifying the target molecules.
3. The method according to paragraph 2, wherein the amplifying comprises nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), or ramification amplification method (RAM).
4. The method according to paragraph 2, wherein the amplifying is performed with RPA or PCR.
5. The method according to paragraph 1, wherein the target molecules are contained in a biological sample or an environmental sample.
6. The method according to paragraph 5, wherein the sample is from a human.
7. The method according to paragraph 5, wherein the biological sample is blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, or fluid obtained from a joint, or a swab of skin or mucosal membrane surface.
8. The method according to paragraph 1, wherein the one or more guide are RNAs designed to bind to corresponding target molecules comprise a (synthetic) mismatch.
9. The method according to paragraph 8, wherein said mismatch is up- or downstream of a SNP or other single nucleotide variation in said target molecule.
10. The method according to paragraph 1, wherein the one or more guide RNAs are designed to detect a single nucleotide polymorphism in a target RNA or DNA, or a splice variant of an RNA transcript.
11. The method according to paragraph 10, wherein the one or more guide RNAs are designed to detect drug resistance SNPs in a viral infection.
12. The method according to paragraph 1, wherein the one or more guide RNAs are designed to bind to one or more target molecules that are diagnostic for a disease state.
13. The method according to paragraph 12, wherein the disease state is characterized by the presence or absence of drug resistance or susceptibility gene or transcript or polypeptide.
14. The method according to paragraph 1, wherein the one or more guide RNAs are designed to distinguish between one or more microbial strains.
15. The method according to paragraph 12, wherein the disease state is an infection.
16. The method according to paragraph 15, wherein the infection is caused by a virus, a bacterium a fungus, a protozoa, or a parasite.
17. The method according to paragraph 15, wherein the one or more guide RNAs comprise at least 90 guide RNAs.
18. The method according to paragraph 1, wherein the Cas protein is an RNA-targeting protein, a DNA-targeting protein, or a combination thereof.
19. The method according to paragraph 18, wherein the RNA targeting protein comprises one or more HEPN domains.
20. The method according to paragraph 19, wherein the one or more HEPN domains comprise a RxxxxH motif sequence.
21. The method according to paragraph 20, wherein the RxxxH motif comprises a R{N/H/K]X₁X₂X₃H sequence
22. The method according to paragraph 21, wherein X₁ is R, S, D, E, Q, N, G, or Y, and X₂ is independently I, S, T, V, or L, and X₃ is independently L, F, N, Y, V, I, S, D, E, or A
23. The method according to paragraph 1, wherein the CRISPR RNA-targeting protein is C2c2.
24. The method according to paragraph 18, wherein the Cas protein is a DNA-targeting protein.
25. The method according to paragraph 24, wherein the Cas protein comprises a RuvC-like domain.
26. The method according to paragraph 24, wherein the DNA-targeting protein is a Type V protein.
27. The method according to paragraph 24, wherein the DNA-targeting protein is a Cas12.
28. The method according to paragraph 25, wherein the Cas12 is Cpf1, C2c3, C2c1 or a combination thereof.
29. The method according to paragraph 1, wherein the masking construct is RNA-based and suppresses generation of a detectable positive signal.
30. The method according to paragraph 29, wherein the RNA-based masking construct suppresses generation of a detectable positive signal by masking the detectable positive signal, or generating a detectable negative signal instead.
31. The method according to paragraph 29, wherein the RNA-based masking construct comprises a silencing RNA that suppresses generation of a gene product encoded by a reporting construct, wherein the gene product generates the detectable positive signal when expressed.
32. The method according to paragraph 29, wherein the RNA-based masking construct is a ribozyme that generates the negative detectable signal, and wherein the positive detectable signal is generated when the ribozyme is deactivated.
33. The method according to paragraph 32, wherein the ribozyme converts a substrate to a first color and wherein the substrate converts to a second color when the ribozyme is deactivated.
34. The method according to paragraph 29, wherein the RNA-based masking agent is an RNA aptamer and/or comprises an RNA-tethered inhibitor.
35. The method according to paragraph 34, wherein the aptamer or RNA-tethered inhibitor sequesters an enzyme, wherein the enzyme generates a detectable signal upon release from the aptamer or RNA tethered inhibitor by acting upon a substrate.
36. The method according to paragraph 34, wherein the aptamer is an inhibitory aptamer that inhibits an enzyme and prevents the enzyme from catalyzing generation of a detectable signal from a substrate or wherein the RNA-tethered inhibitor inhibits an enzyme and prevents the enzyme from catalyzing generation of a detectable signal from a substrate.
37. The method according to paragraph 36, wherein the enzyme is thrombin, protein C, neutrophil elastase, subtilisin, horseradish peroxidase, beta-galactosidase, or calf alkaline phosphatase.
38. The method according to paragraph 37, wherein the enzyme is thrombin and the substrate is para-nitroanilide covalently linked to a peptide substrate for thrombin, or 7-amino-4-methylcoumarin covalently linked to a peptide substrate for thrombin.
39. The method according to paragraph 34, wherein the aptamer sequesters a pair of agents that when released from the aptamers combine to generate a detectable signal.
40. The method according to paragraph 29, wherein the RNA-based masking construct comprises an RNA oligonucleotide to which a detectable ligand and a masking component are attached.
41. The method according to paragraph 29, wherein the RNA-based masking construct comprises a nanoparticle held in aggregate by bridge molecules, wherein at least a portion of the bridge molecules comprises RNA, and wherein the solution undergoes a color shift when the nanoparticle is disbursed in solution.
42. The method according to paragraph 41, wherein the nanoparticle is a colloidal metal.
43. The method according to paragraph 42, wherein the colloidal metal is colloidal gold.
44. The method according to paragraph 22, wherein the RNA-based masking construct comprising a quantum dot linked to one or more quencher molecules by a linking molecule, wherein at least a portion of the linking molecule comprises RNA.
45. The method according to paragraph 22, wherein the RNA-based masking construct comprises RNA in complex with an intercalating agent, wherein the intercalating agent changes absorbance upon cleavage of the RNA.
46. The method according to paragraph 45, wherein the intercalating agent is pyronine-Y or methylene blue.
47. The method according to paragraph 22, wherein the detectable ligand is a fluorophore and the masking component is a quencher molecule.
48. The method according to paragraph 1, wherein the detecting the optical barcodes comprises making optical assessments of the droplets in each microwell.
49. The method according to paragraph 48, wherein the making optical assessments comprises capturing an image of each microwell.
50. The method according to paragraph 1, wherein the optical barcode comprises a particle of a particular size, shape, refractive index, color, or combination thereof.
51. The method according to paragraph 50, wherein the particle comprises colloidal metal particles, nanoshells, nanotubes, nanorods, quantum dots, hydrogel particles, liposomes, dendrimers, or metal-liposome particles.
52. The method according to paragraph 48, wherein the optical barcode is detected using light microscopy, fluorescence microscopy, Raman spectroscopy, or a combination thereof.
53. The method according to paragraph 1, wherein each optical barcode comprises one or more fluorescent dyes.
54. The method according to paragraph 53, wherein each optical barcode comprises a distinct ratio of fluorescent dyes.
55. The method according to paragraph 1, wherein the detectable signal is a level of fluorescence.
56. The method according to paragraph 1, further comprising the step of applying a set cover solving process.
57. The method according to paragraph 1, wherein the microfluidic device comprises an array of at least 40,000 microwells.
58. The method according to paragraph 57, wherein the microfluidic device comprises an array of at least 190,000 microwells.
59. A multiplex detection system comprising:
a detection CRISPR system comprising an RNA targeting protein and one or more guide RNAs designed to bind to corresponding target molecules, an RNA-based masking construct and an optical barcode;
optional optical barcodes for one or more target molecules;
and a microfluidic device comprising an array of microwells and at least one flow channel beneath the microwells, the microwells sized to capture at least two droplets.
   60. A kit comprising the multiplex detection system according to paragraph 59.
   61. The method of any according to paragraphs 1-58, wherein the second set of droplets comprises an optical barcode.
   62. The multiplex detection system according to paragraph 59, wherein the system comprises optical barcodes for one or more target molecules.

The invention is further described in the following examples, which do not limit the scope of the invention described in the claims.

### EXAMPLE METHODS

In an exemplary method, compounds can be mixed with a unique ratio of fluorescent dyes. Each mixture of target molecule with a dye mixture can be emulsified into droplets. Similarly, each detection CRISPR system with optical barcode was emulsified into droplets. In some embodiments, the droplets are approximately 1 nL each. The droplets can then be combined and applied to the microwell chip. The droplets can be combined by simple mixing. In one exemplary embodiment, the microwell chip is suspended on a platform such as a hydrophobic glass slide with removable spacers that can be clamped from above and below by clamps, for example, neodymium magnets. The gap between the chip and the glass created by the spacers can be loaded with oil, and the pool of droplets injected into the chip, continuing to flow the droplets by injecting more oil and draining excess droplets. After loading is completed, the chip can be washed with oil to purge free surfactant. Spacers can be removed to seal microwells against the glass slide and clamp closed. The chip is then imaged with an epifluorescence microscope, then droplets merged to mix the compounds in each microwell by applying an AC electric field, for example, supplied by corona treater. Incubation of microwells at 37° C. with measurement of fluorescence using epifluorescence microscope.

Regarding design of primers, the following exemplary method for viral sequences can be utilized, utilizing “diagnostic-guide-design” method implemented in a software tool. In the case of viral sequences, an input of an alignment of viral sequences is utilized and its objective is to find a set of guide sequences, all within some specified amplicon length, that will detect some desired fraction (e.g., 95%) of the input sequences tolerating some number of mismatches (usually 1) between the guide and target. Critically for subtyping (or any differential identification), it designs different collections of guides guaranteeing that each collection is specific to one subtype. The goal is to build on this to simultaneously design amplicon primers and guide sequences for species identification using diagnostic-guide-design (“d-g-d”) together with other tools:

Assemble requisite viral genomes, make an alignment at the species level with mafft, cluster the data to identify closely related species. Treat segmented viruses specially; each segment is treated separately. Ultimately, pick the best segment (or two) to proceed with.

Use diagnostic-guide-design to identify putative primer-binding sites (25mers). Look for a single primer sequence, with 95% coverage and no more than 2 mismatches allowed.

If there is no way to achieve this coverage at a position/window, move on to the next position, performing this across the whole genome first before calling primer3

Identify pairs of primers for amplicons between 80 and 120 nucleotides in length. Use primer3 to narrow down the 25mer to get a target melting temperature of 58-60 C.

Use SEQUENCE_PRIMER_PAIR_OK_REGION_LIST to specify fwd/reverse primer locations for putative amplicons. This allows one to input regions where primers can go using [fwd_start, fwd_length, rev_start, rev_length] format.

Preferably, PCR can be run at a lower temperature, for example, between 50 and 55 C.

If the primer has bad secondary structure, throw it out (PRIMER_MAX_SELF_ANY_TH, PRIMER_PAIR_MAX_COMPL_ANY_TH set to 40 C). This is lower than the default setting of 47 C, but stringency is desired here to get good primers.

Check the amplicons for cross-reactivity using the clustering data. This can be done using primer3, which allows for a “mispriming library” that primers are supposed to avoid. One can feed in a list of sequences from other species (but in the same cluster) here. It's possible that an amplicon could have unique primers, but still have overlap at the crRNA leve, necessary to ensure that the assays are very specific.

Pass those amplicons to d-g-d and try and find crRNAs

Allowing 1 mismatch, as have done before

Window size is the entire amplicon (with no overlap to the primer sequences)

Do differential design using the clustering data (probably just checking amplicons vs. other amplicons as unamplified material should be scarce). Require at least 4 mismatches (not including G-U pairs).

Come up with a list of amplicons that have few crRNAs, high coverage, and are specific

Right now, a single “best” design can be prepared but the code needs to be modified to allow e.g. whitelisting to give several options to test for each virus

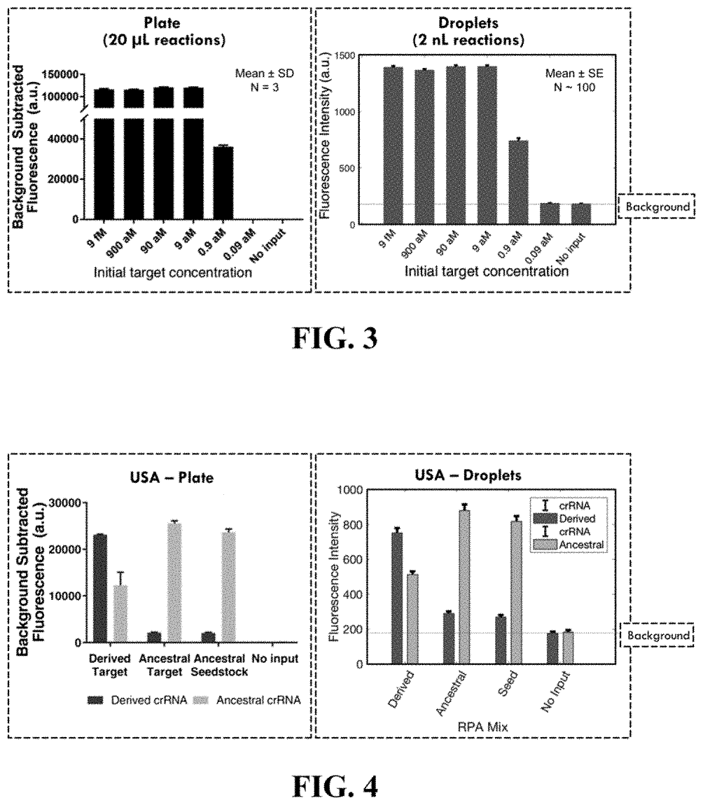

The sensitivity curve for the same Zika samples analyzed by SHERLOCK for Zika virus in plates using 20 uL reactions is the same as a SHERLOCK assay for Zika virus in droplets using a 2 nL reaction, indicating droplet SHERLOCK (dSHERLOCK) limit-of detection is comparable to plates. (FIG. 3). Similarly, dSHERLOCK discriminates single nucleotide polymorphisms (SNPs) equally well when compared to assay in plates.

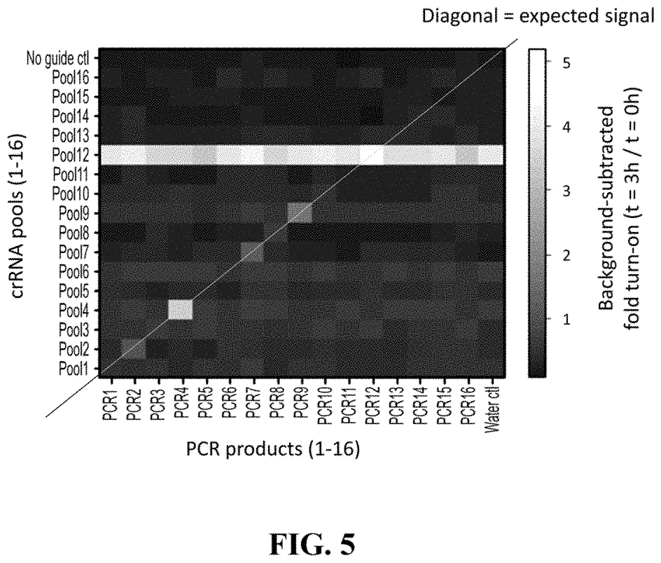

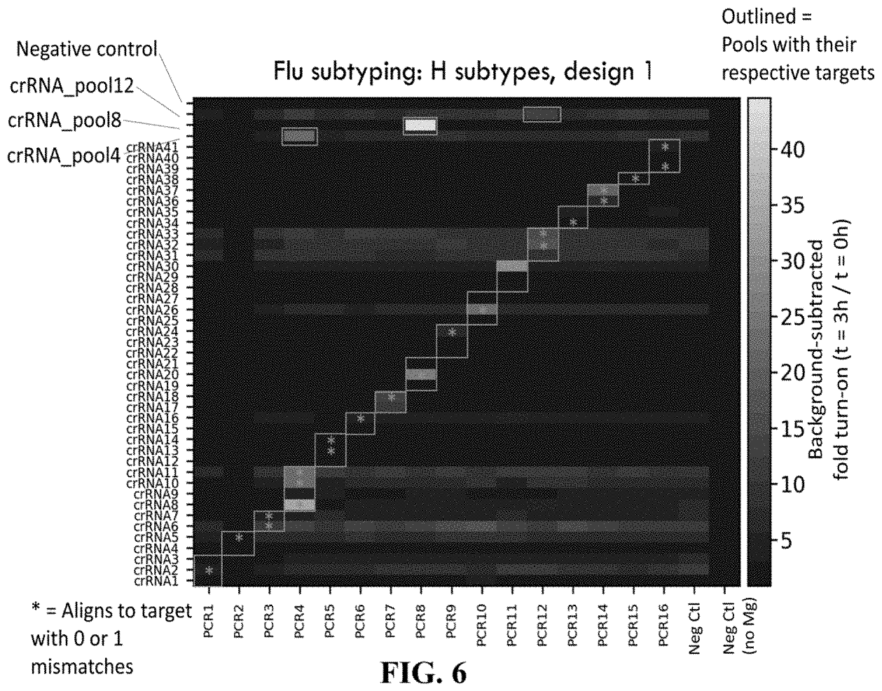

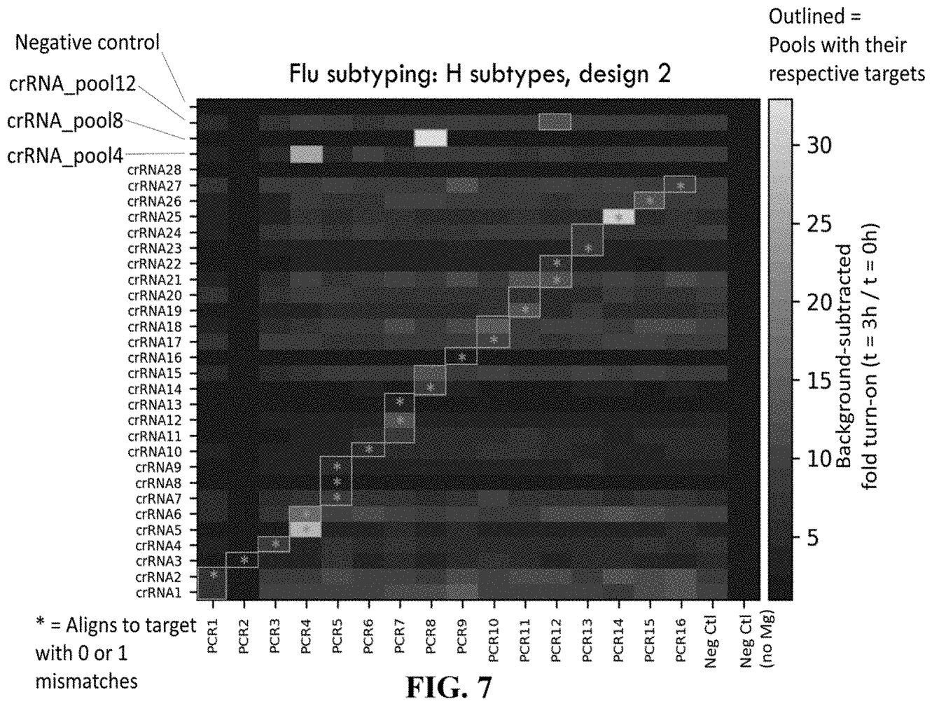

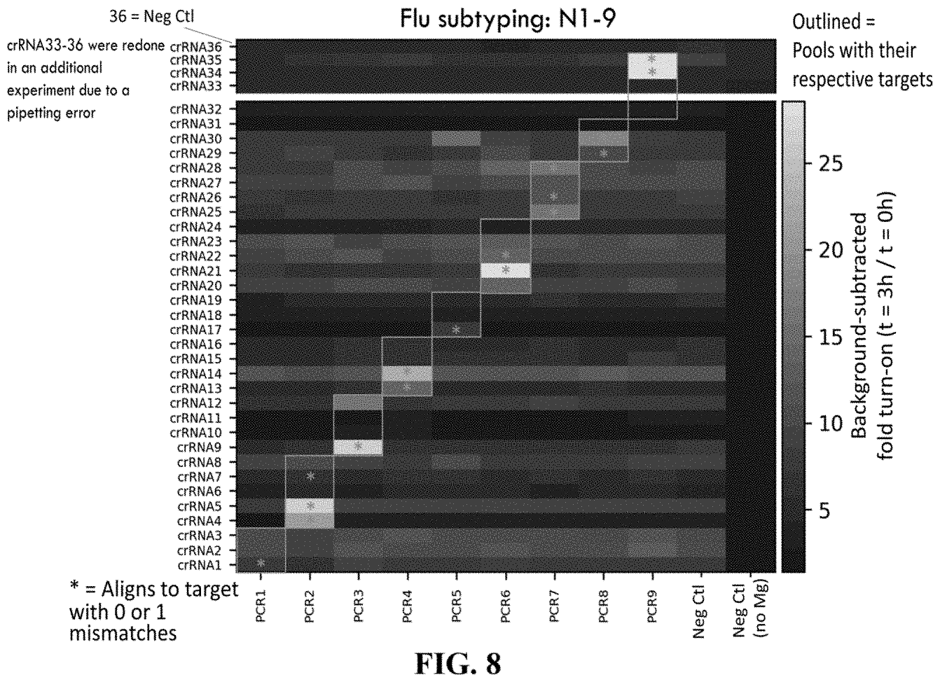

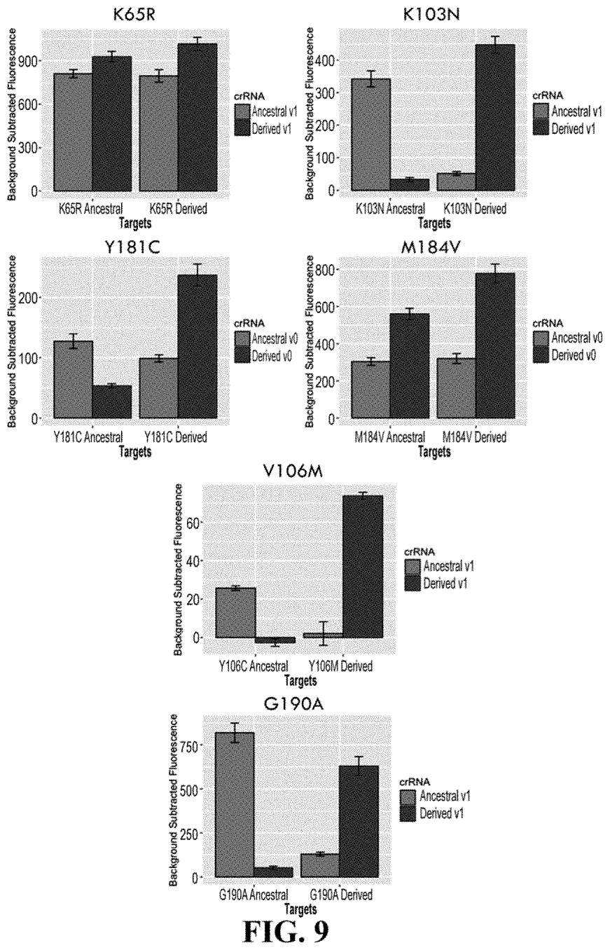

The methods and systems disclosed herein can be utilized for the multiplexed detection of Influenza subtypes (FIG. 5). Notably, the experimental effort required to generate all combinations of detection mixes and targets in the chip is the same as the effort necessary to construct just the on-diagonal reactions in a well-plate, which allows the systems and methods to be applied to analytics with large numbers of combinations. Because the chip automatically constructs all off-diagonal combinations in addition to the diagonal, rapid determination of the selectivity of each detection mix for its intended product is achievable. Guide RNAs can be designed to target particular unique segments of a virus based on sequences deposited. In some instances, the design can be weighted to include more recent sequence data, or more prevalent sequences. Sets of guide RNAs can be designed against various viral subtypes, as is shown in FIG. 6 for Influenza H subtypes, with successful results providing alignment of guide RNAs to majority consensus sequence for each subtype with 0 or 1 mismatches.

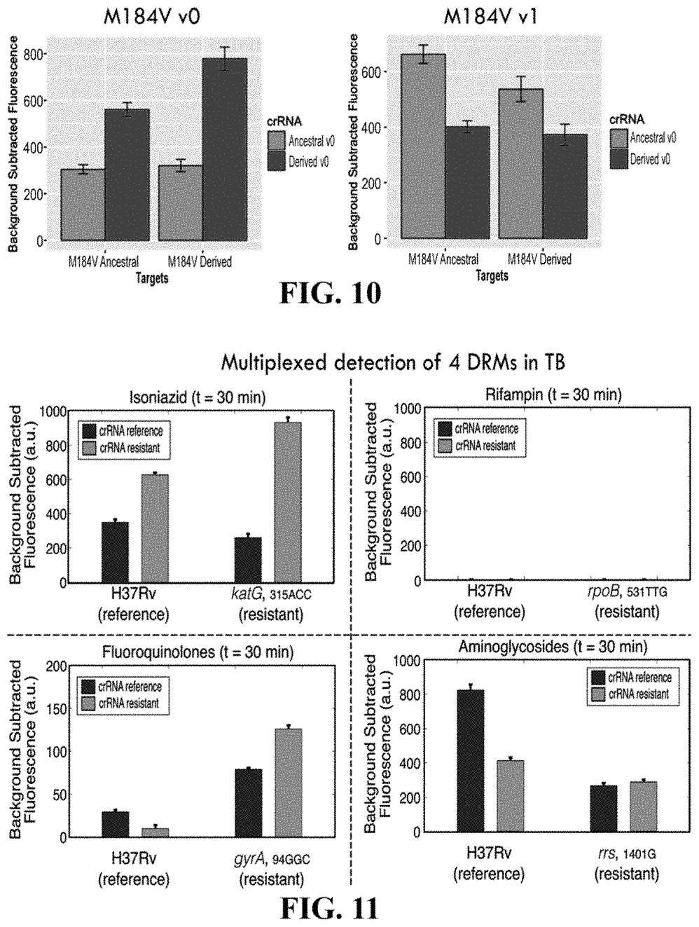

Other exemplary applications of the current systems and methods include multiplexed detection of mutations, including detection of drug resistance mutations in TB (FIG. 11) and in HIV reverse transcriptase. Guide RNAs can be designed to target ancestral and derived alleles, with tests showing the potential to use tests for derived and target alleles together. (FIG. 10). dSHERLOCK can be performed with fluorescence detected within 30 minutes. (FIG. 11).

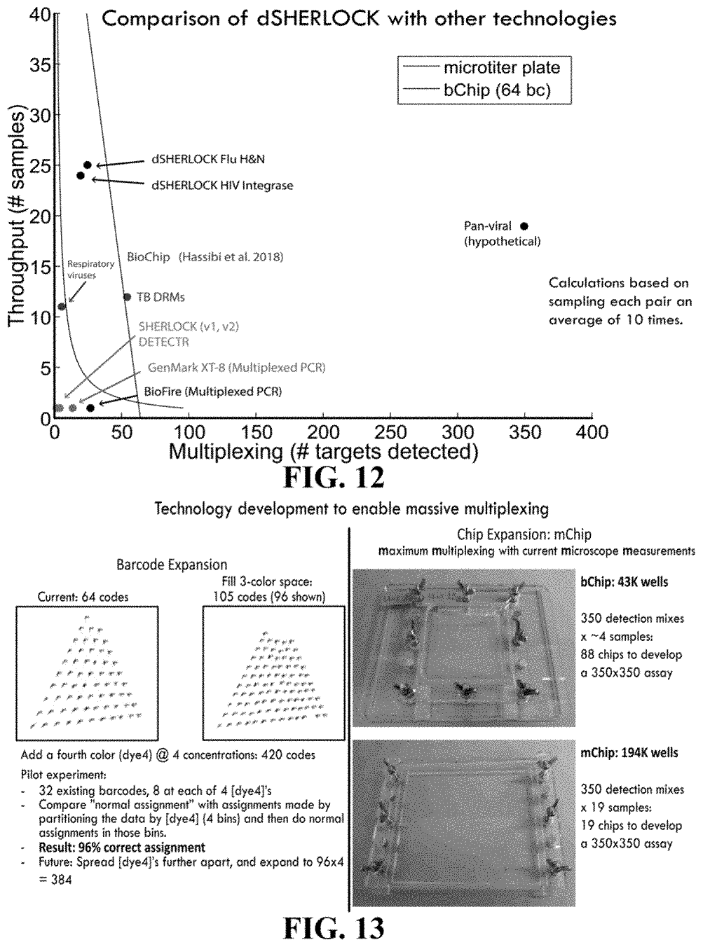

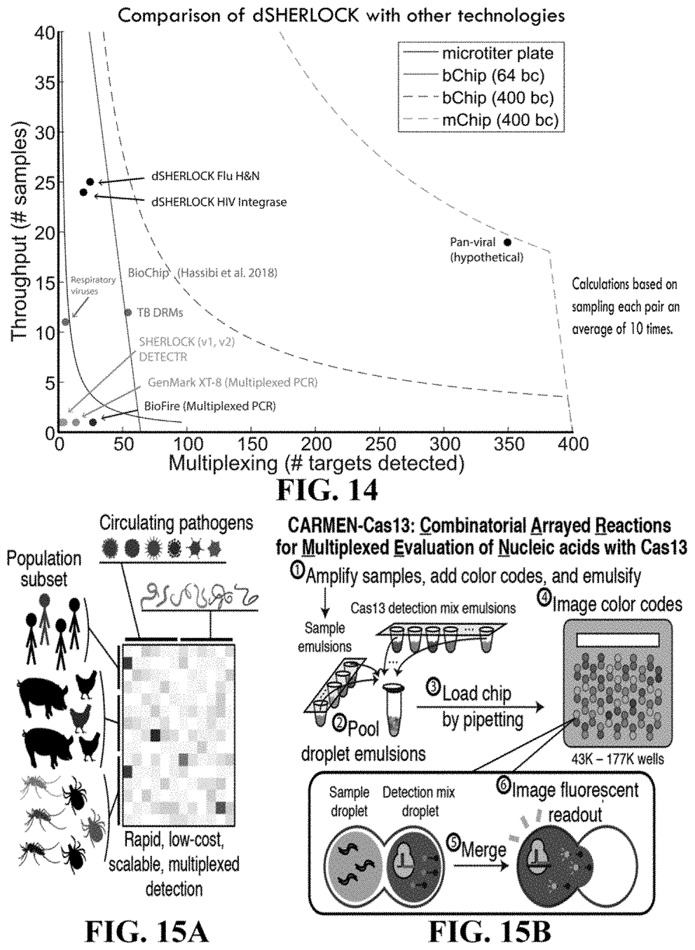

Combining SHERLOCK in the methods disclosed herein, using microwell array chips and droplet detection can provide the highest throughput for multiplexed detection to date, with expansion of the number of barcodes and chip size enabling massive multiplexing. (FIGS. 12-14).

### Working Example 1

The example describes development of Combinatorial Arrayed Reactions for Multiplexed Evaluation of Nucleic acids (CARMEN) and implementation of CARMEN using Cas13 (CARMEN-Cas13). As shown herein, CARMEN-Cas13 specifically, selectively, and simultaneously tested dozens of samples for all human-associated viruses with ≥10 sequenced genomes. Additionally, CARMEN-Cas13 capitalizes on the sensitivity and specificity of Cas13 detection to discriminate all strains of a diverse viral species in parallel and detect panels of single nucleotide variants such as drug-resistance mutations. In summary, CARMEN-Cas13 is a highly multiplexed CRISPR-based nucleic acid detection platform that can enable epidemiological surveillance at unprecedented scale.

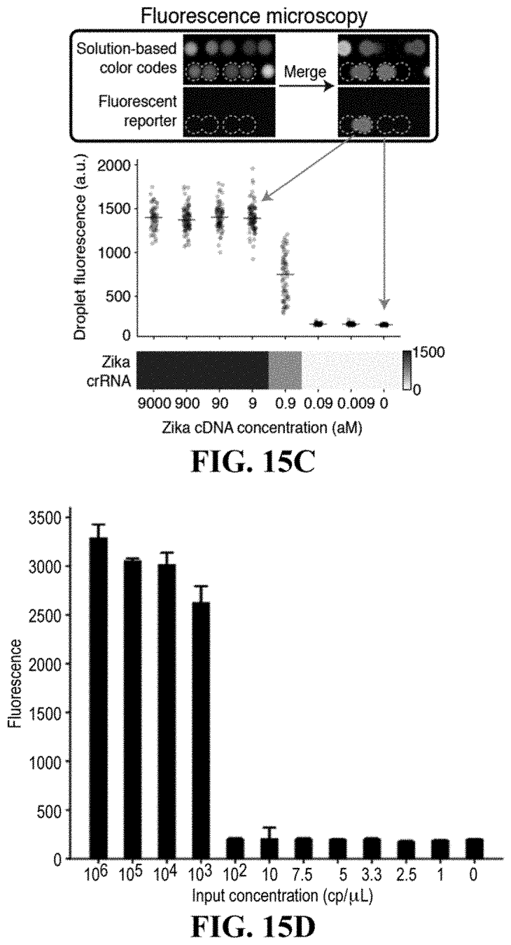

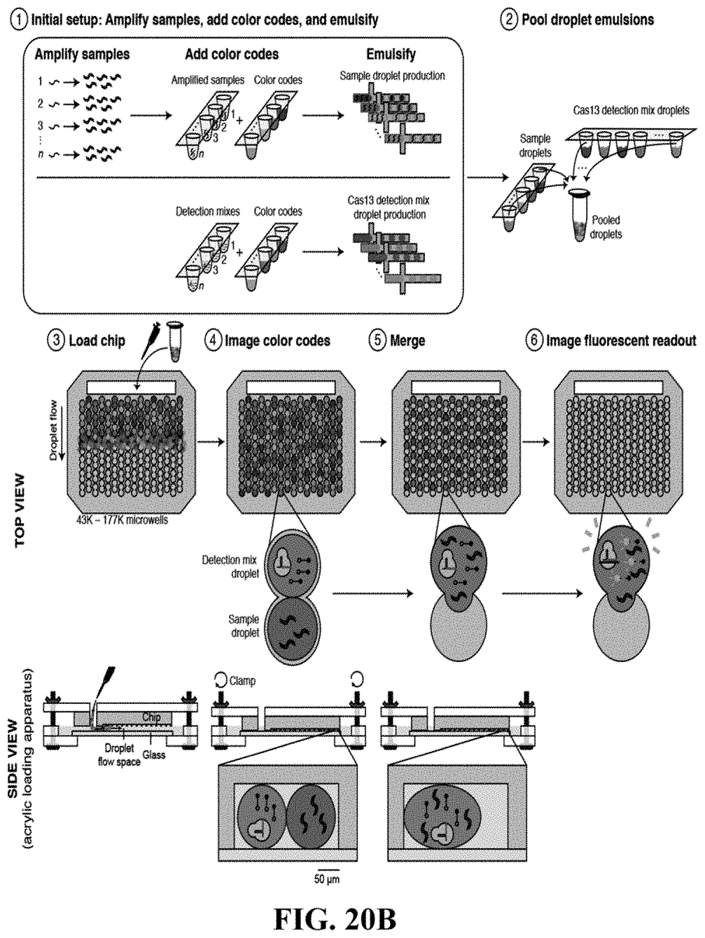

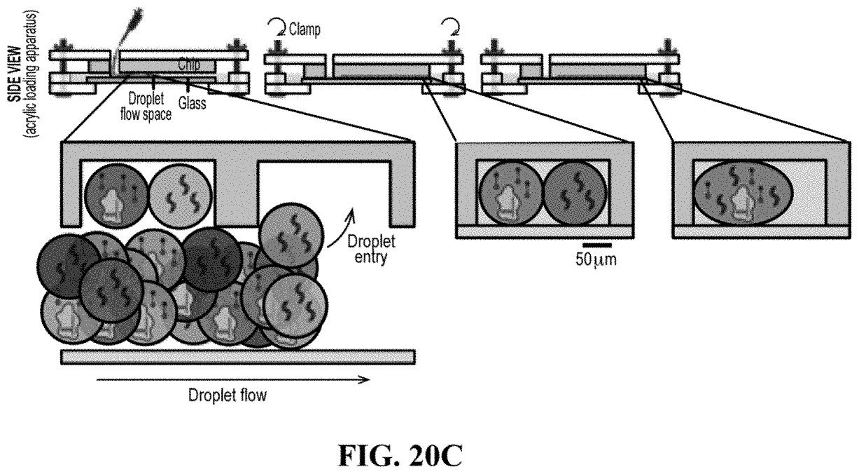

CARMEN transforms conventional CRISPR-based nucleic acid detection into a multiplexed assay by confining each sample and detection mix to emulsified droplets and constructing sample-detection mix pairs in a microwell array (FIG. 15B, FIG. 20). Amplified samples and detection mixes are prepared in conventional microtiter plates. Each amplified sample or detection mix is combined with a distinct fluorescent color code that serves as a unique optical identifier, and the color-coded solutions are emulsified in fluorous oil to yield 1 nL droplets. Once emulsified, droplets from all samples and detection mixes are pooled into a single tube and, in a single pipetting step, are loaded into a microwell array built into a polydimethylsiloxane (PDMS) chip (FIG. 15B and FIG. 20-21). Each microwell in the array accommodates two droplets from the pool at random, thereby spontaneously forming all pairwise combinations of dropletized inputs, and the array is physically sealed against glass substrate to physically isolate each microwell. The contents of each well are determined by evaluating the color codes of the droplets using fluorescence microscopy. Exposure to an electric field merges the droplet pairs confined in each microwell and initiates all detection reactions simultaneously. Fluorescence microscopy is used to monitor each detection reaction over time (FIG. 15B and FIG. 20).

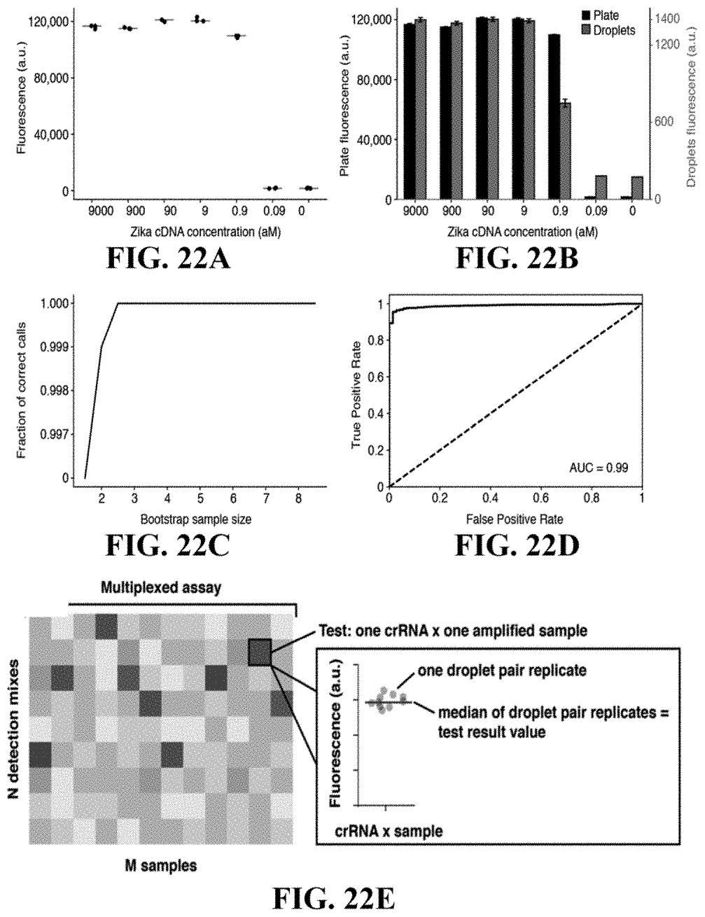

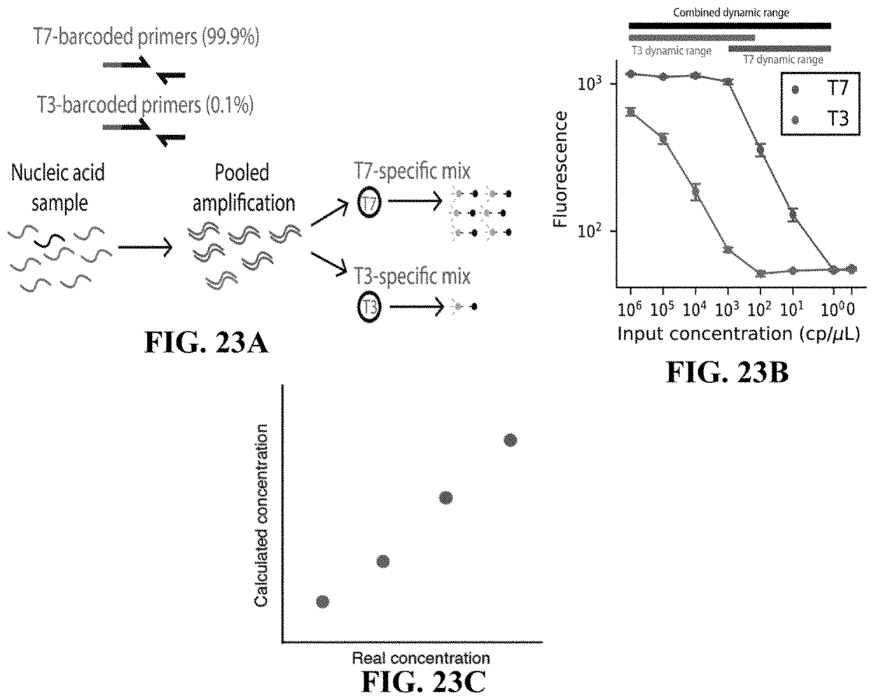

CARMEN-Cas13 is as sensitive as Specific High sensitivity Enzymatic Reporter unLOCKing (SHERLOCK), which has been used to rapidly detect a variety of viral and bacterial pathogens in complex samples, and the large number of data points collected per microwell array can be used to adjust statistical power versus throughput in each experiment. CARMEN-Cas13 detects Zika sequences with attomolar sensitivity, matching the sensitivity of standard SHERLOCK and PCR-based assays (FIG. 15C and FIG. 22). Moreover, performing CARMEN on Applicants standard chip, data are obtained from ˜10,000 microwells after quality filtering, providing the potential for hundreds of technical replicates per test (FIG. 15C). Bootstrap analysis shows that CARMEN-Cas13 is highly consistent, requiring only 3 technical replicates per test (FIG. 20). Performing up to 1,000 tests per chip ensures that >X % of pairs have 3 or more technical replicate droplets pairs per test. The geometry of the combinatorial space (eg. 100 samples×10 detection mixes, or 10 samples×100 detection mixes) is flexible. One application of CARMIEN's flexibility is to increase the dynamic range of nucleic acid detection by evaluating multiple parallel detection reactions containing orthogonal RNA polymerases. To demonstrate this principle, amplification primers were barcoded using orthogonal RNA polymerase promoters, T3 and T7, and detection reactions were used containing either T3 or T7 RNA polymerase to generate a standard curve over 6 orders of magnitude (FIG. 23).

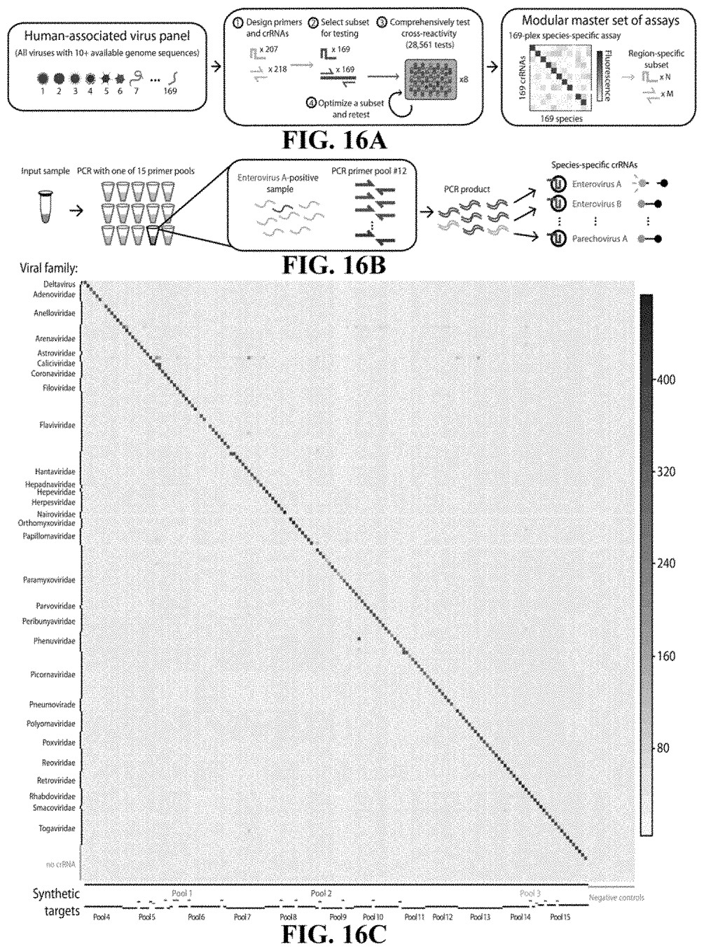

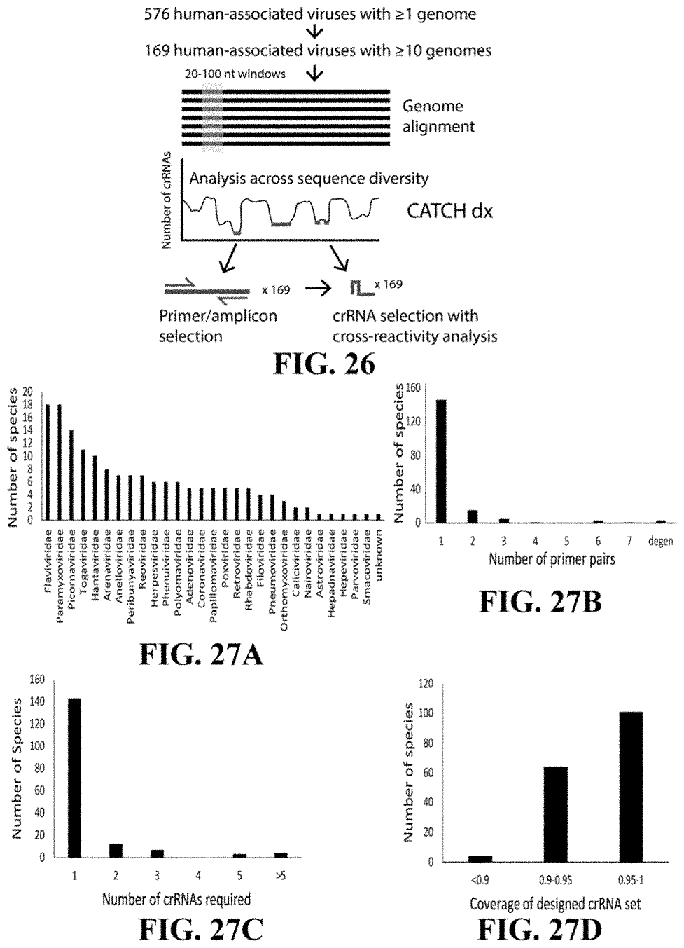

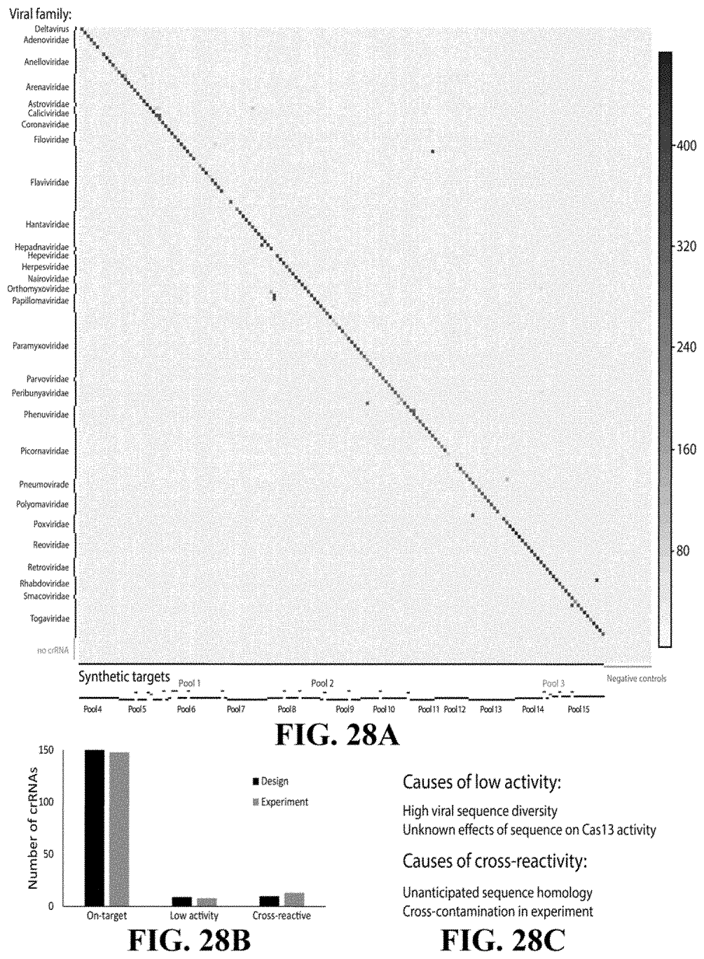

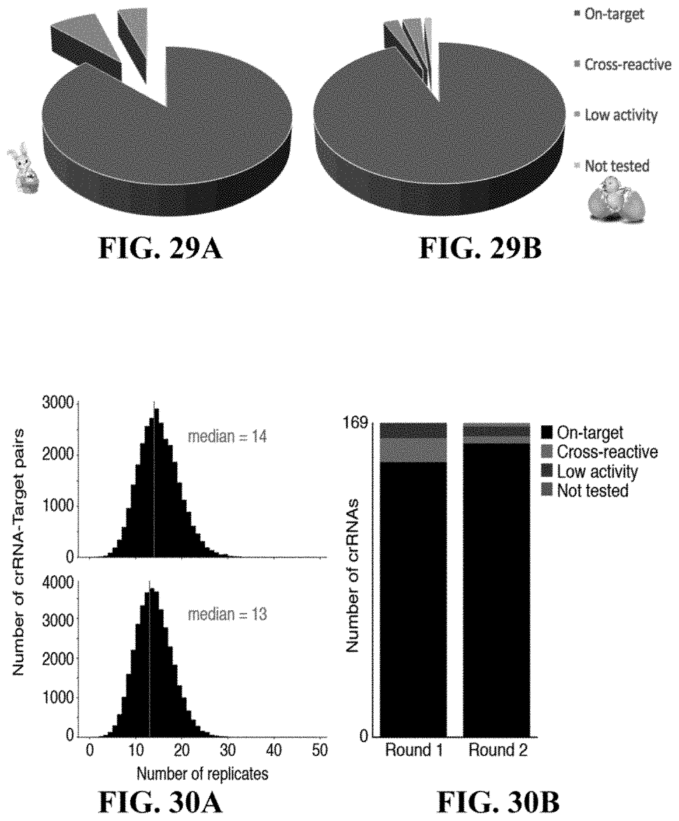

Beyond quantification, CARMEN enables multiplexed nucleic acid detection at unprecedented scale. To showcase this scale, the next focus was to design an assay that could specifically, selectively and simultaneously test dozens of samples for all 169 human-associated viruses with ≥10 published genomes to inform the design of a Cas13 detection assay (FIG. 16A, FIG. 26). Only 39 of these species have FDA-approved diagnostics, due in large part to the labor-intensive process of developing and validating such tests. Applicants undertook development of a CARMEN assay to identify each of these 169 viral species simultaneously.

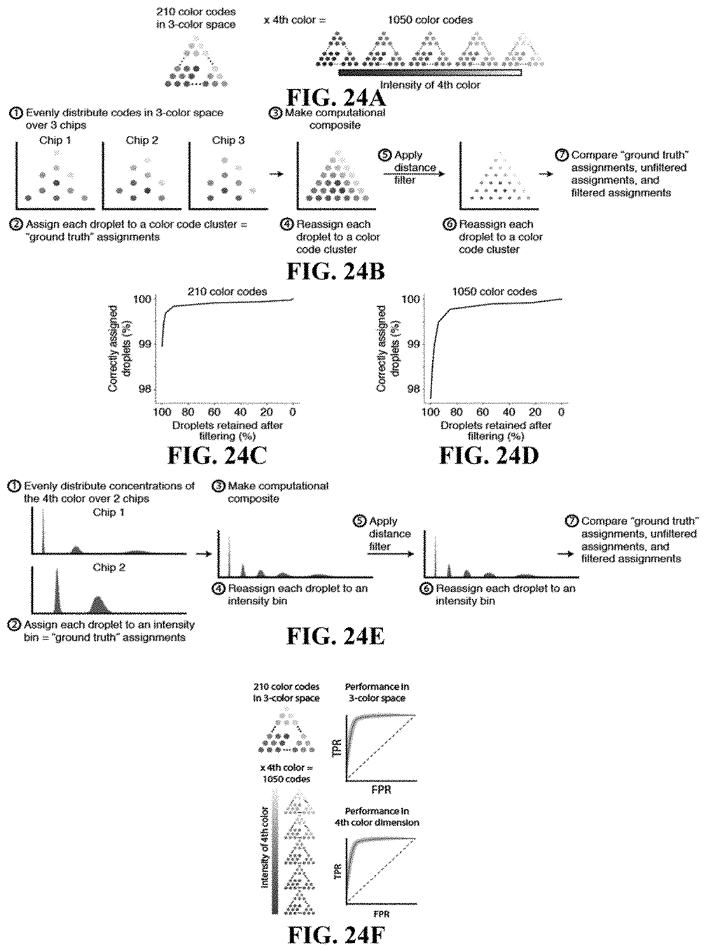

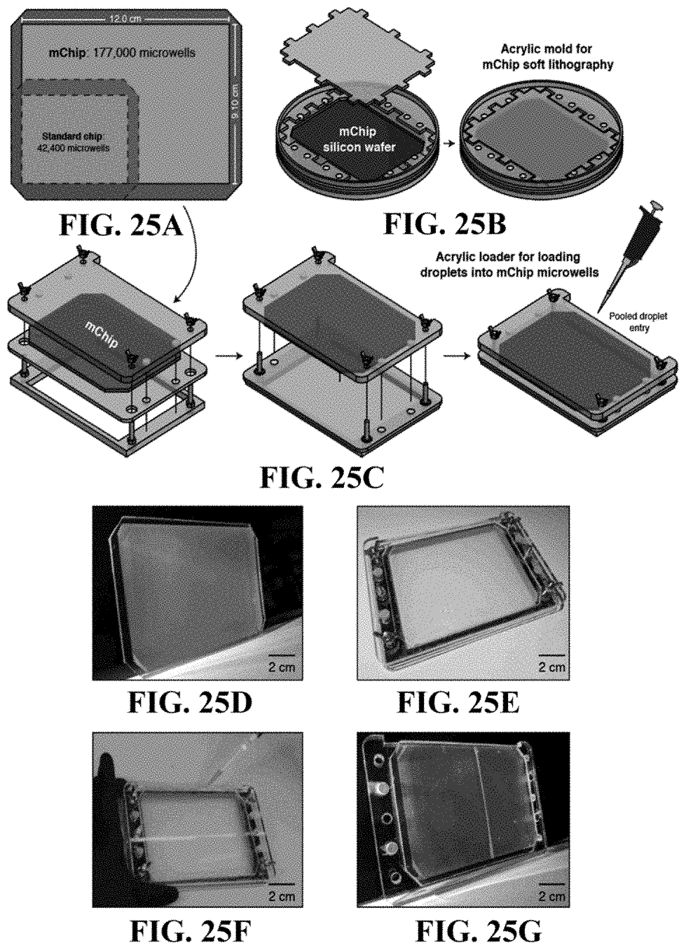

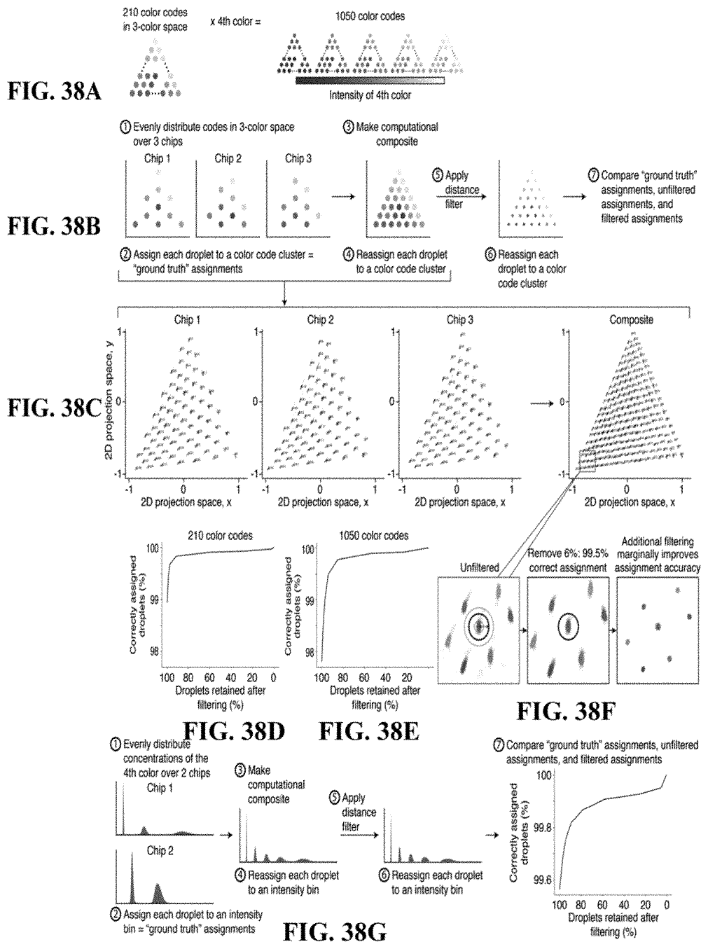

The experimental effort to develop and test an assay to span the human-associated virome (169 samples×169 detection mixes=28,561 tests, before controls and replicates) demanded higher throughput than previous standard chip and color code set and other existing multiplex systems can offer. In order to differentiate droplets from hundreds of inputs, Applicants developed a set of 1,050 solution-based color codes using ratios of 4 commercially-available, small-molecule fluorophores, building significantly on the existing 64 color code set⁸ without requiring custom particle synthesis previously reported for highly multiplexed and precise spectral encoding systems²⁴⁻²⁶. The 1,050 color codes performed comparably to the original set, with 97.8% correct droplet classification across all droplets and 99.5% correct classification after permissive filtering that retained 94% of droplets (FIG. 24, FIG. 16B, FIG. 38A-38G). With as few as 5 replicates, the chance of misclassified droplets leading to a miscalled test is 1 in 100,000. To match the throughput enabled by the expanded color code set, Applicants designed a larger capacity chip (mChip)(FIG. 25A-25G) that has 4x more surface area than the previous standard chip, allowing >4,000 robust and statistically replicated tests to be performed simultaneously. mChip reduces the reagent cost per test >300-fold relative to standard well-plate SHERLOCK tests. (Table 11).

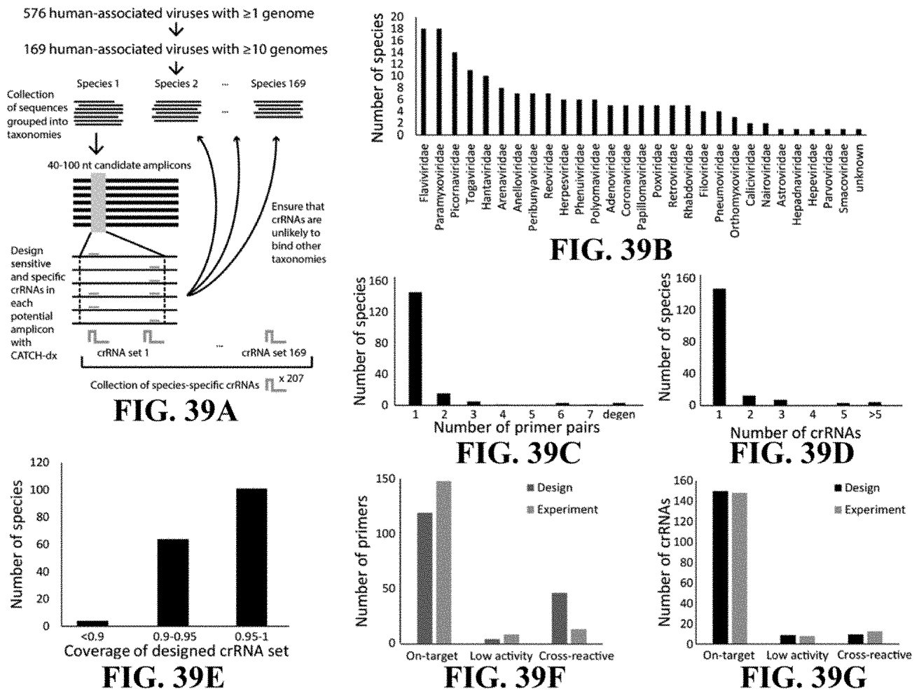

Applicants next designed a CARMEN-Cas13 assay that could selectively and simultaneously test dozens of samples for all 169 human-associated viruses (HAVs) with ≥10 available, published genomes applied CATCH-dx (Metsky et al. in prep) to the published viral genomes of viruses represented in the HAV panel to select amplicons for PCR primer pools, using primer3 to optimize primer sequences²⁷. CATCH-dx accepts a collection of sequences arranged into groups (e.g., all known sequences within a species). For each group, CATCH-dx searches for an optimal set of crRNAs that are sensitive to the sequences within the group (i.e., detect a desired fraction of sequences) and are unlikely to detect sequences in the other groups (FIG. 39A). With alignments of viral species as an input, CATCH dx was used to design a small set of crRNA sequences for each species such that, accounting for genome diversity on NCBI GenBank, each set provides high sensitivity (>90% of sequences detected) within its targeted species and high selectivity against other species (FIG. 16C, FIG. 26; FIG. 39A-39G). The design was tested using synthetic targets based on the consensus sequences for each species, and the optimal crRNA from each species set in the design was computationally selected for testing. (FIG. 16B).

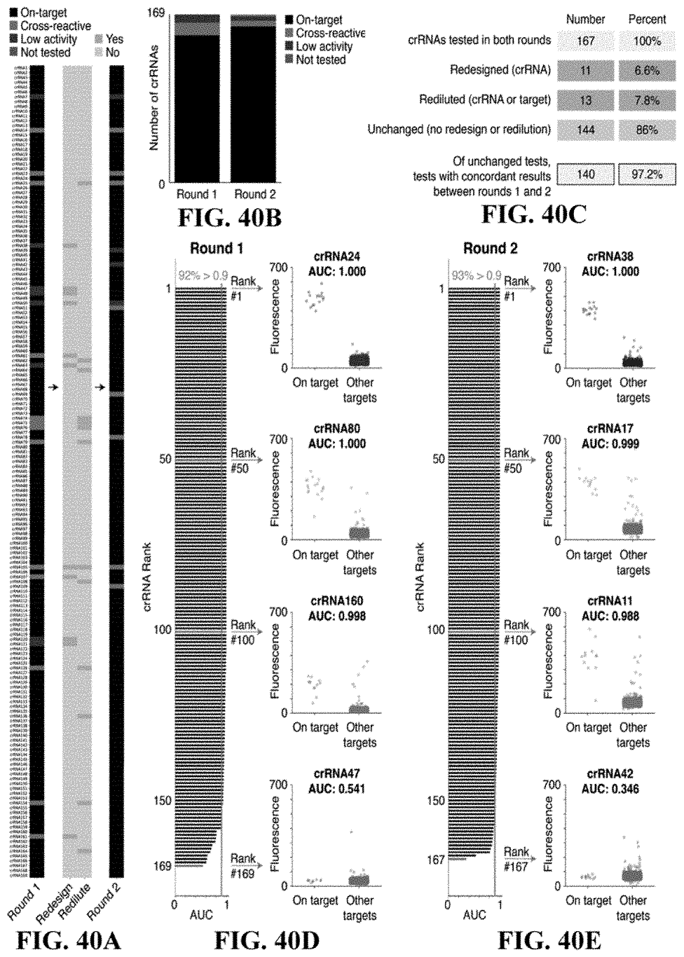

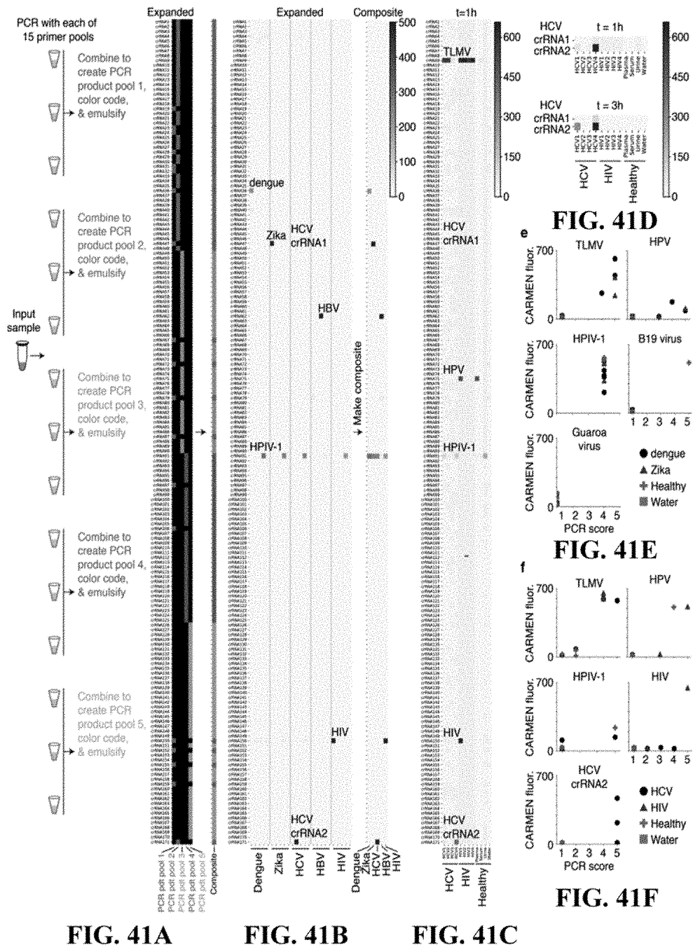

Taking advantage of CARMEN-Cas13's massive multiplexing capabilities, Applicants extensively tested the HAV panel, demonstrating high performance. Each crRNA (169 total) was evaluated against all targets each of which had been amplified using its corresponding primer pool (184 total PCR products, including controls; FIG. 16B), for a total of 30,912 tests performed across 8 mChips (see Table 1). In an initial design set, 148 crRNAs (87.6%) were already highly selective for their targets, with signal above threshold, 13 (7.7%) showed cross-reactivity above threshold, and 8 (4.7%) exhibited no reactivity above threshold. To address underperforming crRNAs, crRNA sequences for 11 species were redesigned, primer sequences for 3 species were redesigned, and fresh stocks of crRNAs and targets were prepared. In a second round of testing that incorporated the redesigned sequences, 157 of 167 crRNAs evaluated (94%) were highly selective for their targets, with signal above threshold, 6 (3.6%) showed cross-reactivity above threshold, and 4 (2.4%) had no reactivity above threshold (FIG. 16C). The results of rounds 1 and 2 were remarkably concordant: 97.2% of sequences that were neither redesigned nor rediluted performed equivalently between the two rounds, demonstrating that individual crRNAs can be improved without altering the performance of the rest of the assay (FIG. 40A-40E). Furthermore, the performance of individual crRNAs is strong (median AUCs of 0.999 and 0.997 for rounds 1 and 2, respectively) (FIG. 40A-40E). Indeed, widespread cross-reactivity is not observed, even when synthetic targets are amplified with all primer pools (FIG. 41A-41F).

To rigorously test the performance of CARMEN in a more challenging and complex context, Applicants evaluated the HAV panel against plasma or serum samples from 16 patients with confirmed infections. Each clinical sample was treated as an unknown and amplified using all 15 primer pools. To increase testing throughput, PCR products were subsequently pooled in sets of 3 (5 final products per patient sample) and tested with crRNAs from the HAV panel. As a comparative readout, a second round of PCR was performed with species-specific PCR primers. CARMEN and PCR amplification were 100% concordant for dengue, Zika, and HIV samples. For HCV, a highly diverse virus, the HCV-specific crRNAs in the HAV panel identified 2 of 4 PCR-positive samples. Sensitivity of detection, especially for diverse viruses, can be addressed with increased multiplexing of crRNAs to cover the heterogeneous target set, as demonstrated with influenza A subtyping in FIG. 3 below. Furthermore, the specificity of CARMEN is high, and cross-reactivity is not wide-spread. Only 3 of 169 crRNAs (1.8%) displayed unexpected reactivity in 3 diverse negative controls (pooled plasma, serum, or urine from healthy humans), results that were 89.6% concordant with PCR amplification. Those 3 crRNAs were removed from the analysis without influencing the performance of the rest of the HAV panel.

In addition to identifying the individual causes of symptomatic infections, the HAV panel can be used for surveillance of many viruses in parallel. Here, the HAV panel identified Torque teno-like mini virus (TLMV) and a strain of human papillomavirus (HPV) in a subset of patients (TLMV: 11/16 patients, HPV: 4/16 patients); these results were confirmed by a second round of PCR with 100% concordance. These viruses are known to commonly infect people, are often asymptomatic, and frequently go undiagnosed, demonstrating that multiplexed CARMEN panels can be used to identify secondary or subclinical infections. In clinical settings, integrating results from the HAV panel with patient symptoms is critical for interpretation and results may only be needed from a subset of the HAV panel. The HAV panel can therefore be considered a modular master set of nucleic acid detection assays which can be customized by the end user for diverse applications.

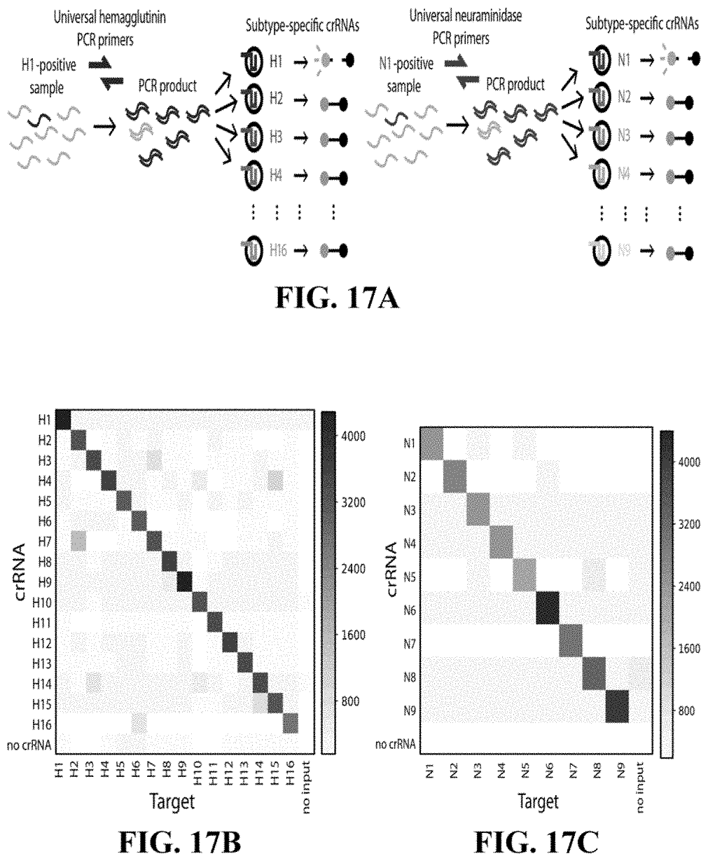

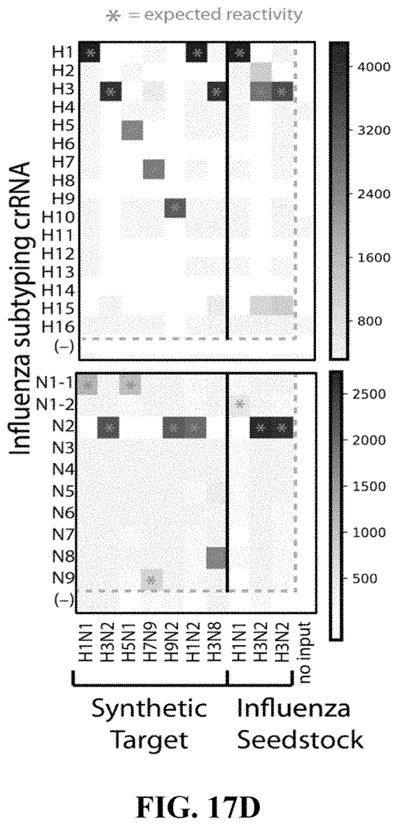

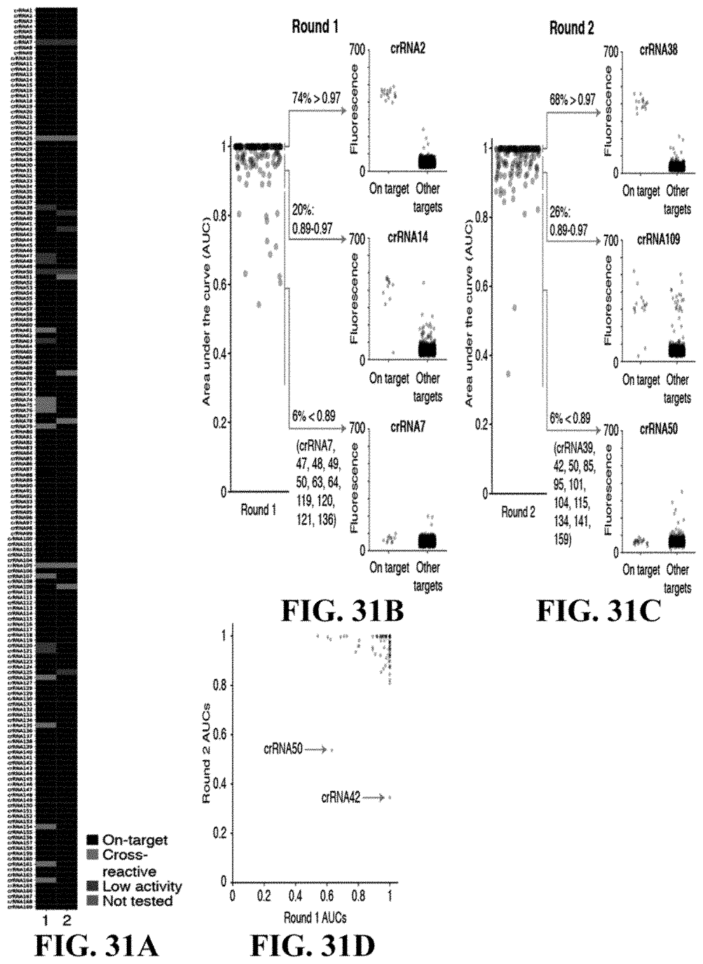

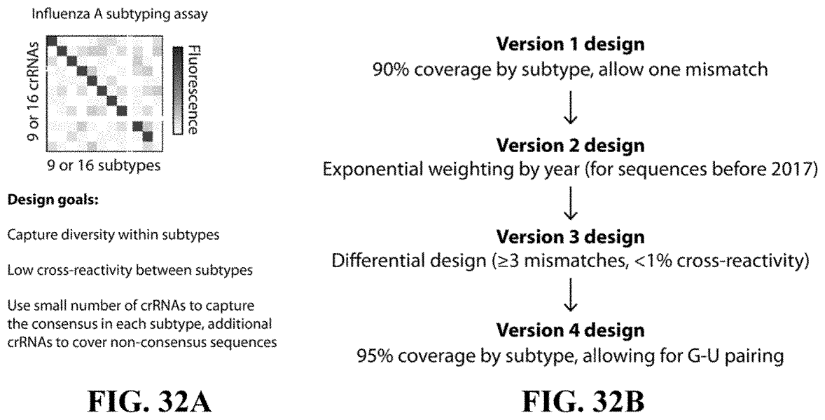

Capitalizing on the specificity of Cas13 detection, Applicants used CARMEN-Cas13 to discriminate all epidemiologically relevant serotypes of a diverse viral species in parallel. diverse viral strains in parallel. Diversity within a viral species poses a significant challenge to detection: an assay must correctly identify many distinct sequences within a group of strains, while remaining selective for that group. As a case study, hemagglutinin (H) and neuraminidase (N) subtypes H1-H16 and N1-N9 of influenza A virus (IAV) were chosen. These serologically defined subtypes consist of strains capable of infecting a wide variety of host species, some of which are associated with pandemic potential. H and N amplicons were identified that were sufficiently conserved to amplify with parallel primer sets. To identify subtypes, CATCH dx was used to design specific sets of crRNAs to cover >90% of the sequences within each subtype (FIG. 17A, FIG. 30, see Methods for details). The optimal crRNA was tested from each set using synthetic consensus sequences from H1-16 and N1-9, and readily identified these subtypes (FIG. 17B-17C, FIG. 31). The N subtyping assay was further tested using 35 synthetic sequences representing >90% of the sequence diversity within each N subtype, and determined that 32 out of 35 (91.4%) of these sequences could be identified (FIG. 32). The subtyping assay was also validated using seedstocks from H1N1 and H3N2 strains, the subtypes of IAV that commonly circulate in humans, and synthetic sequences from avian IAV subtypes (FIG. 17D, Table 1). Based on these results, the assay could potentially identify any of the 144 possible combinations of H1-16 and N1-9 subtypes.

TABLE 1
Droplet pairing and filtering statistics for testing of the human associated virus panel, rounds 1 and 2

| | | Droplet | crRNA + Target | Yield | Filtered | Passed | | | |
|---|---|---|---|---|---|---|---|---|---|
| | | pairs | pairs | (%) | pairs | filter (%) | crRNAs | Targets | Tests |
| Testing | Chip1 | 154,451 | 74,518 | 48.2 | 67,773 | 90.9 | 22 | 200 | 4400 |
| round 1 | Chip2 | 154,331 | 74,344 | 48.2 | 65,868 | 88.6 | 22 | 200 | 4400 |
| | Chip3 | 156,621 | 75,657 | 48.3 | 69,308 | 91.6 | 23 | 200 | 4600 |
| | Chip4 | 157,090 | 75,734 | 48.2 | 67,377 | 89.0 | 22 | 200 | 4400 |
| | Chip5 | 151,248 | 72,694 | 48.1 | 68,311 | 94.0 | 19 | 190 | 3610 |
| | Chip6 | 142,738 | 67,744 | 47.5 | 63,156 | 93.2 | 19 | 190 | 3610 |
| | Chip7 | 141,292 | 67,143 | 47.5 | 63,048 | 93.9 | 19 | 190 | 3610 |
| | Chip8 | 155,889 | 75,361 | 48.3 | 71,141 | 94.4 | 18 | 190 | 3420 |
| Total | | 1,213,660 | 583,195 | | 535,982 | | | | |
| Average | | 151,708 | 72,899 | 48.0 | 66,998 | 92.0 | | | |
| (per chip) | | | | | | | | | |
| Testing | Chip1 | 146,333 | 67,286 | 46.0 | 62,282 | 92.6 | 23 | 189 | 4347 |
| round 2 | Chip2 | 151,635 | 71,971 | 47.5 | 67,212 | 93.4 | 24 | 189 | 4536 |
| | Chip3 | 127,437 | 58,993 | 46.3 | 54,364 | 92.2 | 23 | 189 | 4347 |
| | Chip4 | 149,983 | 71,883 | 47.9 | 66,338 | 92.3 | 25 | 190 | 4750 |
| | Chip5 | 152,618 | 72,098 | 47.2 | 67,405 | 93.5 | 26 | 190 | 4940 |
| | Chip6 | 147,409 | 67,605 | 45.9 | 62,696 | 92.7 | 25 | 190 | 4750 |
| | Chip7 | 142,459 | 67,231 | 47.2 | 61,420 | 91.4 | 26 | 190 | 4940 |
| | Chip8 | 145,938 | 68,795 | 47.1 | 62,701 | 91.1 | 26 | 190 | 4940 |
| Total | | 1,163,812 | 545,862 | | 504,418 | | | | |
| Average | | 145,477 | 68,233 | 46.9 | 63,052 | 92.4 | | | |
| (per chip) | | | | | | | | | |
| Grand Total | | 2,377,472 | 1,129,057 | | 1,040,400 | | | | |
| Average | | 148,592 | 70,566 | 47.5 | 65,025 | 92.2 | | | |
| (per chip) | | | | | | | | | |
| Expected | | 177,000 | 88,500 | 50 | 88,500 | | | | |
| (per chip) | | | | | | | | | |
| Performance | | 84 | 80 | 95 | 73 | | | | |
| (%) | | | | | | | | | |

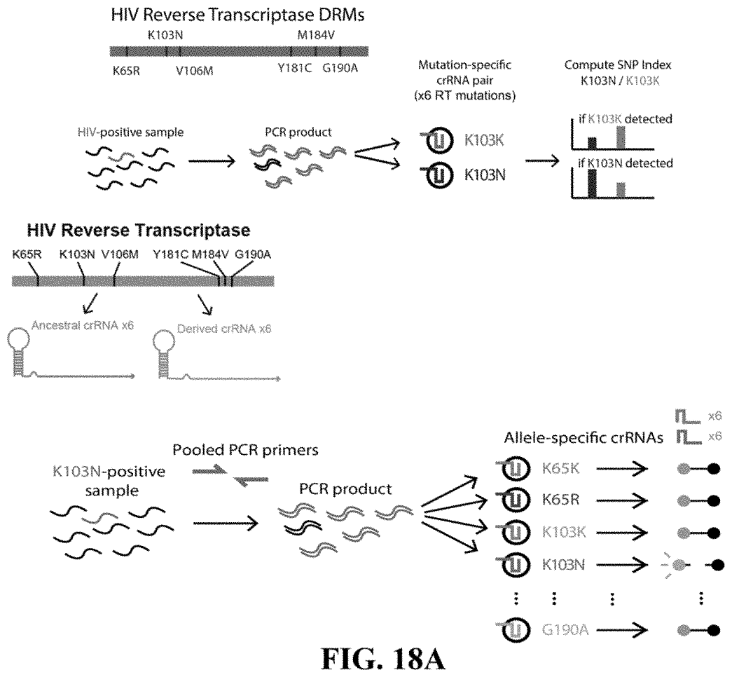

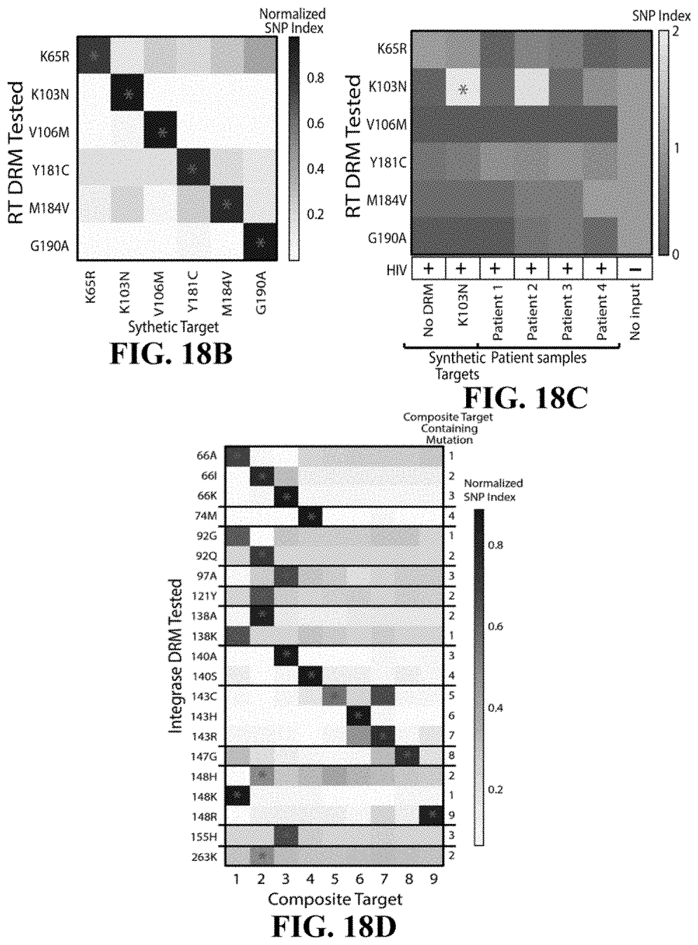

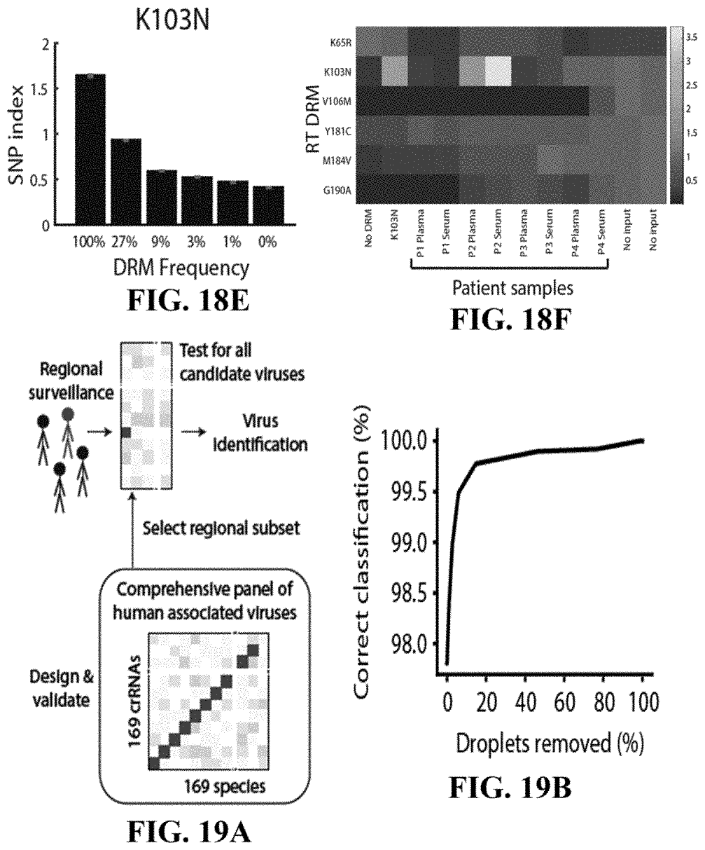

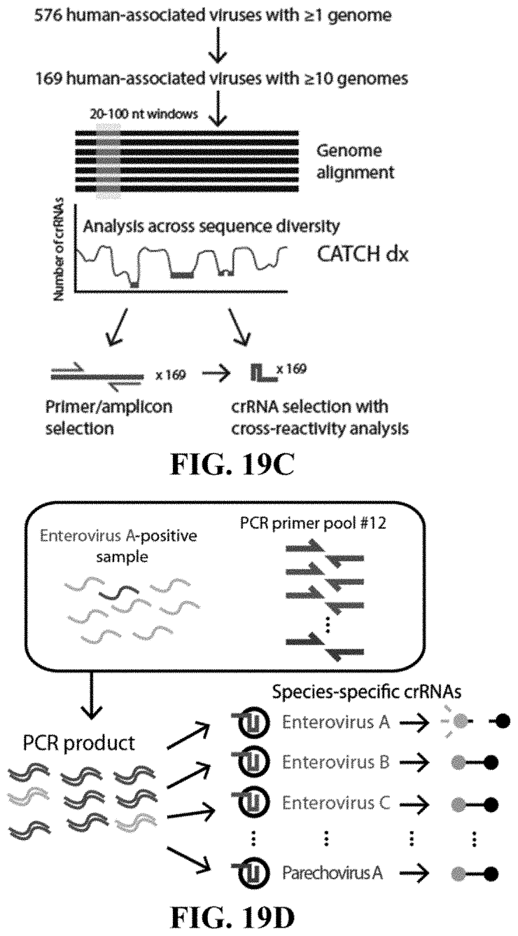

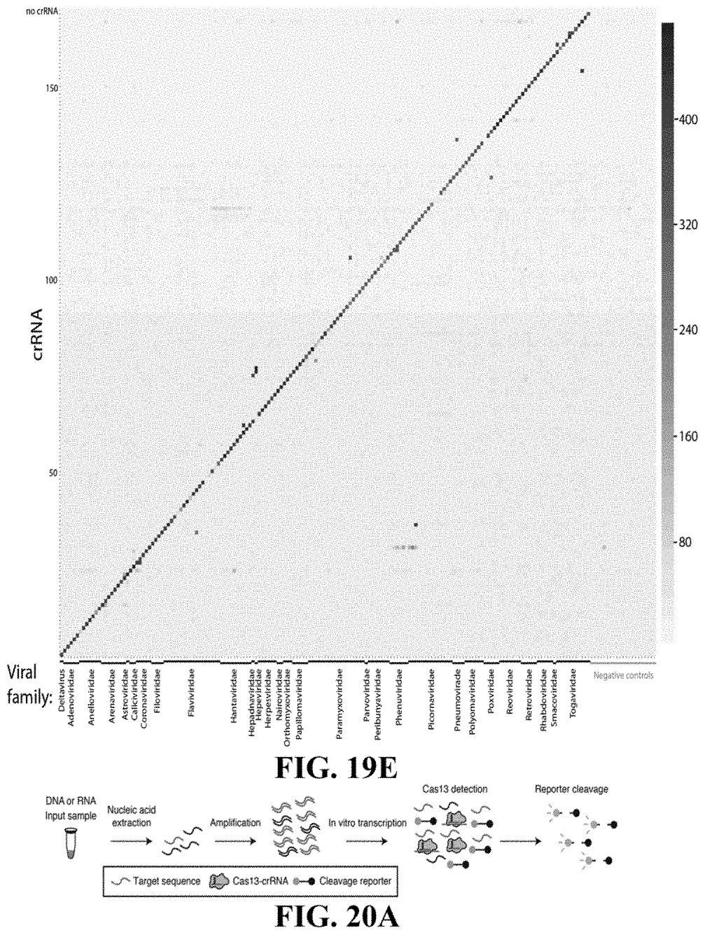

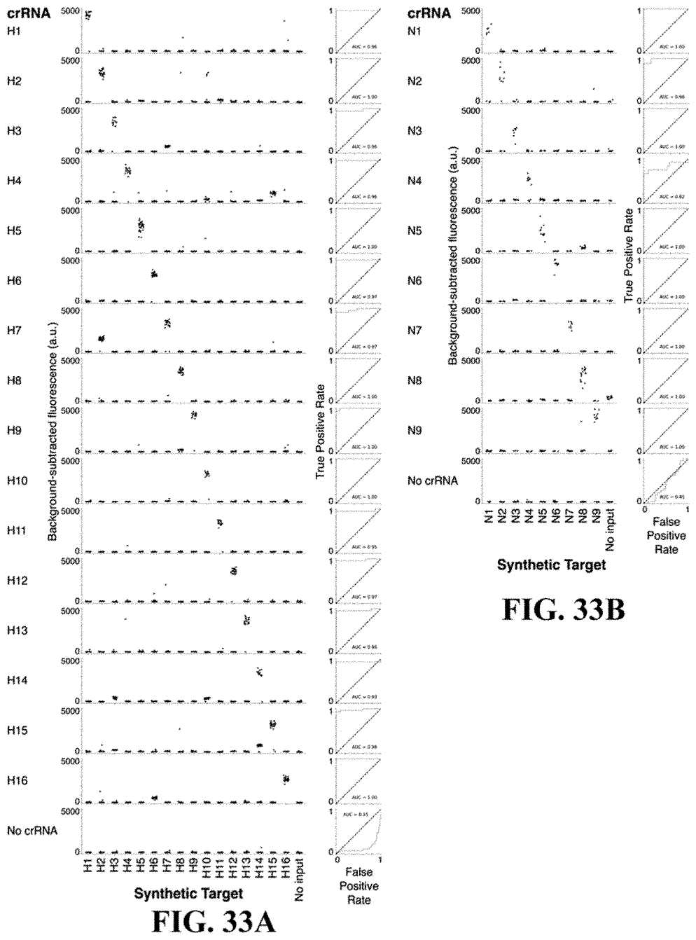

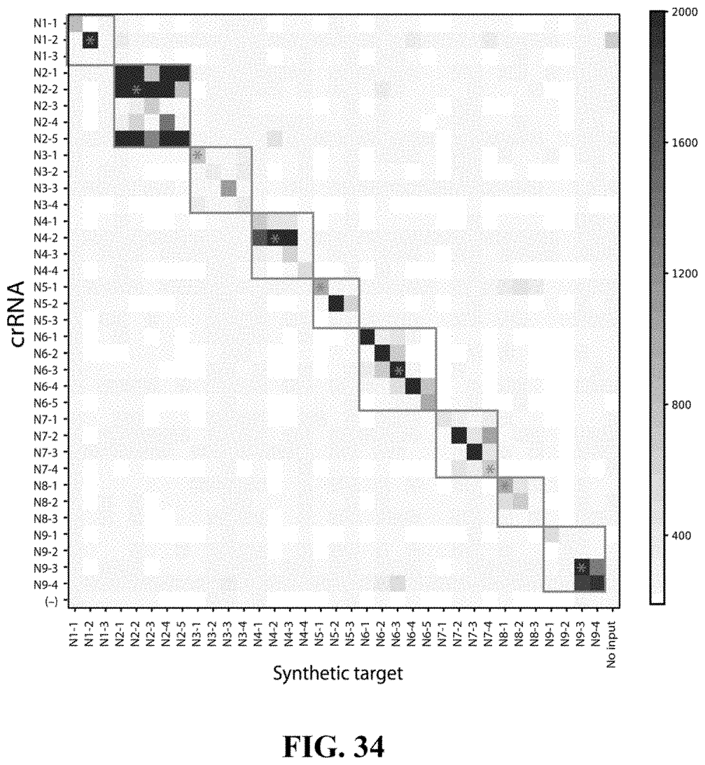

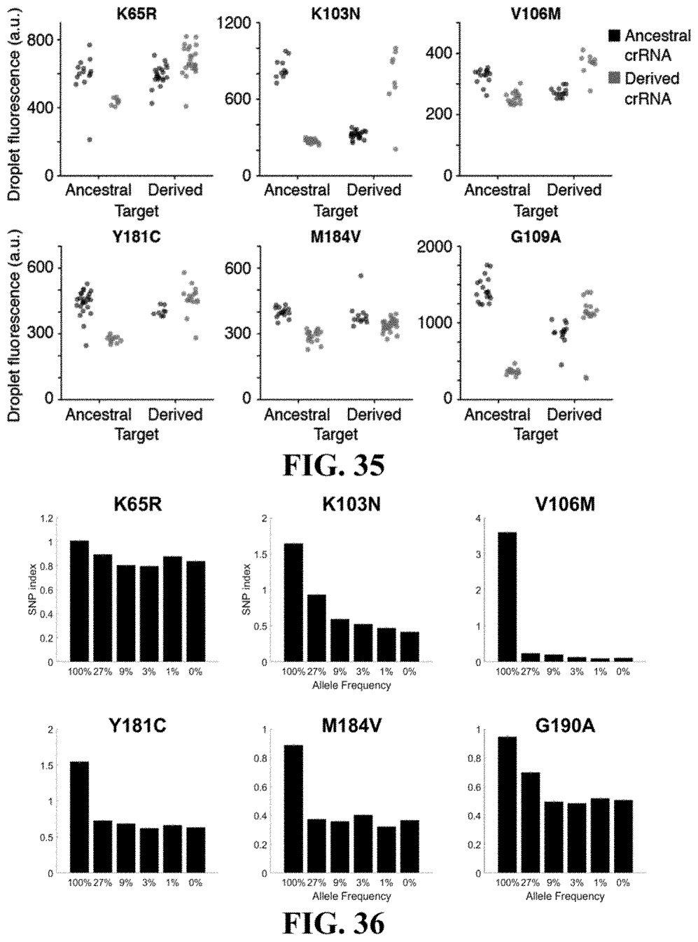

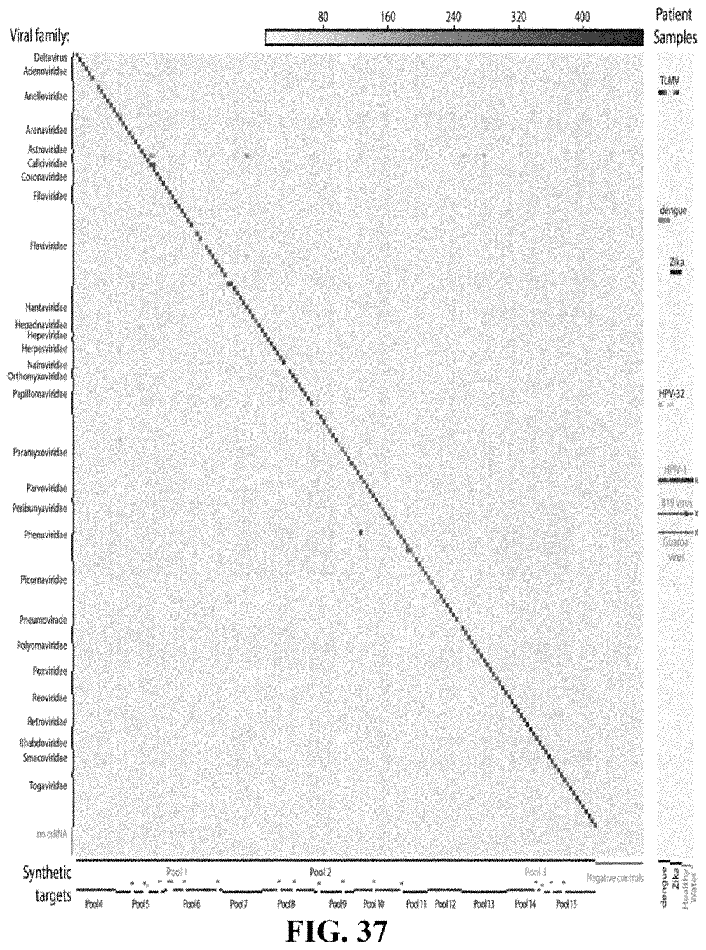

The exquisite specificity of Cas13 enables CARMEN-Cas13 to identify clinically relevant viral mutations in multiplex, such as those that confer drug resistance. As a proof of concept, primer pairs were designed tiling the HIV reverse transcriptase (RT) coding sequence and a set of crRNAs to identify six prevalent drug resistance mutations (DRMs, FIG. 18A, Table 2). These DRMs are prevalent at frequencies ranging from 5-15% in antiviral-naive patient populations in Africa, Latin America, and Asia. The designs were tested designs using synthetic targets, and could identify all 6 mutations in parallel (FIG. 18B, FIG. 33). Applicants further analyzed the performance of the RT assay to detect DRMs at low allele frequencies, and could detect K103N at 1% frequency and other DRMs at 10% frequency (FIG. 34).

Further validation of the RT DRM assay was performed on clinical plasma samples from 4 patients with HIV (FIG. 18D), showing 100% concordance with Sanger-sequencing assays, the gold-standard approach (no DRMs were present in 3 of the 4 patients, and one patient had the K103N mutation). Notably, the CARMEN HIV SNP assay was more sensitive for HIV detection than the HAV panel or the associated PCRs, likely due to higher multiplexing of primers and crRNAs. To demonstrate the generalizability of the approach, Applicants broadened the panel to include a comprehensive set of DRMs in HIV integrase, the target of front-line HIV therapy in high-income countries. Amplification primers and crRNAs were designed to target all 21 integrase DRMs designated as clinically relevant by the International Antiviral Society-USA in 2017. Applicants successfully identified all of these mutations by testing a set of 9 composite synthetic targets (FIG. 18E, Table 2). Of note, 4 of these composite targets contained multiple DRMs, confirming the ability of CARMEN-Cas13 to detect combinations of multiple DRMs simultaneously.

TABLE 2
List of HIV drug-resistance mutations tested for in this study.

| Gene | Mutation |
|---|---|
| Reverse transcriptase | K65R |
| Reverse transcriptase | K103N |
| Reverse transcriptase | V106M |
| Reverse transcriptase | Y181C |
| Reverse transcriptase | M184V |
| Reverse transcriptase | G190A |
| Integrase | 66A |
| Integrase | 66I |
| Integrase | 66K |
| Integrase | 74M |
| Integrase | 92G |
| Integrase | 92Q |
| Integrase | 97A |
| Integrase | 121Y |
| Integrase | 138A |
| Integrase | 138K |
| Integrase | 140A |
| Integrase | 140S |
| Integrase | 143C |
| Integrase | 143H |
| Integrase | 143R |
| Integrase | 147G |
| Integrase | 148H |
| Integrase | 148K |
| Integrase | 148R |
| Integrase | 155H |
| Integrase | 263K |

### DISCUSSION

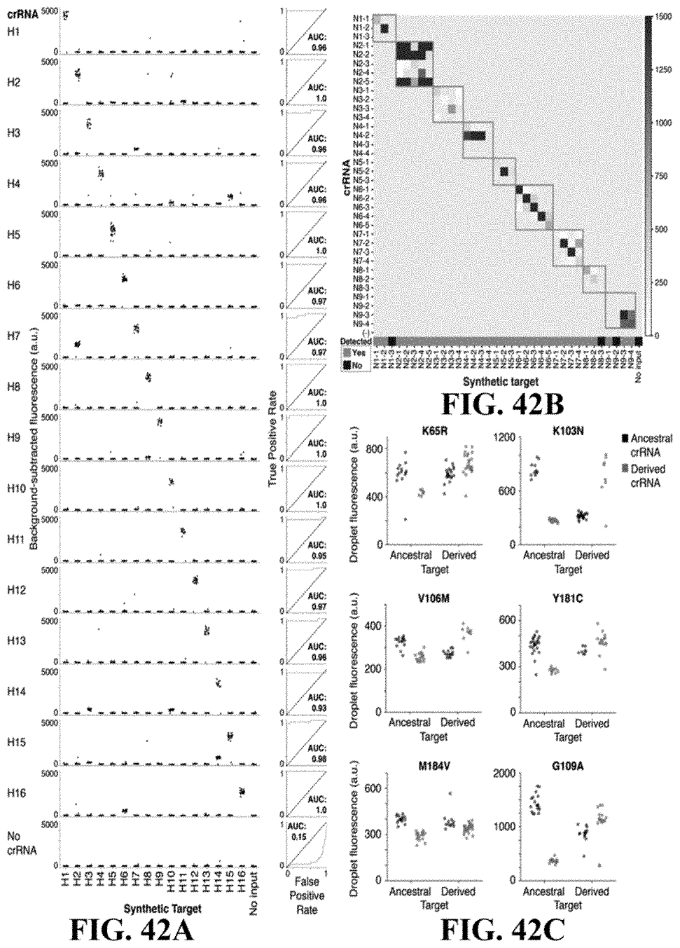

A broad set of uses for CARMEN-Cas13 has been demonstrated—differentiating viral sequences at the species, strain, and SNP levels—and the capability to rapidly develop and validate highly multiplexed detection panels. More generally, CARMEN-Cas13 augments CRISPR-based nucleic acid detection technologies by increasing throughput, decreasing reagent and sample consumption per test, and enabling detection over a larger dynamic range (FIG. 42A-42C). The flexibility and high-throughput of CARMEN can accommodate the addition and rapid optimization of new primers or crRNAs to existing CARMEN assays to facilitate detection of the vast majority of known pathogen sequences. Additionally, in the broader context of pathogen detection, discovery, and evolution, CARMEN and next-generation sequencing complement each other: CARMEN can rapidly identify infected samples that can be further sequenced to track the evolution of the virus, and newly identified sequences can inform the design of improved CRISPR-based diagnostics. Because sequencing data are growing exponentially, one may ultimately create CARMEN assays with near-perfect sensitivity for high-risk pathogens. In the future, Applicants imagine region-specific detection panels deployed to test thousands of samples from selected populations, including animal vectors, animal reservoirs, or patients presenting with symptoms. Routine adoption of such panels will require careful interpretation to make judicious clinical use of the data when human samples are tested. CARMEN unleashes CRISPR-based diagnostics at scale, a critical step toward routine, comprehensive disease surveillance to improve patient care and public health.

### Materials and Methods

Human samples from HIV patients were obtained commercially from Boca Biolistics, and all protocols were approved by the Institutional Review Boards of Massachusetts Institute of Technology (MIT) and Broad Institute of MIT and Harvard.

### General Experimental Procedure

Preparation of Targets, Samples, and crRNAs

Synthetic targets: Synthetic DNA targets were ordered from Integrated DNA Technologies (IDT) and resuspended in nuclease-free water. Resuspended DNA was serially diluted to 10⁴ copies per microliter and used as inputs to PCR reactions.

Sample preparation: For influenza A viral seedstocks and HIV clinical samples, RNA was extracted from 140 μl of input material using the QIAamp Viral RNA Mini Kit (QIAGEN) with carrier RNA according to the manufacturer's instructions. Samples were eluted in 60 μl of nuclease free water and stored at −80° C. until use. 5 μl of extracted RNA was converted into single-stranded cDNA in a 20 μl reaction. First, random hexamer primers were annealed to sample RNA at 70° C. for 7 minutes followed by reverse transcription using SuperScript IV with random hexamer primers for 20 minutes at 55° C., without RNase H treatment. cDNA was stored at −20° C. until use. crRNA preparation: For viral detection (FIGS. 15-18), crRNAs were synthesized by Synthego and resuspended in nuclease-free water. For SNP detection (FIG. 18), crRNA DNA templates were annealed to a T7 promoter oligonucleotide at a final concentration of 10 μM in 1×Taq reaction buffer (New England Biolabs). This procedure involved 5 minutes of initial denaturation at 95° C., followed by an anneal at 5° C. per minute down to 4° C. SNP detection crRNAs were transcribed from annealed DNA templates in vitro using the HiScribe T7 High Yield RNA Synthesis Kit (New England Biolabs). Transcriptions were performed according to the manufacturer's instructions for short RNA transcripts, with the volume scaled to 30 μl. Reactions were incubated for 18 hours or overnight at 37° C. Transcripts were purified using RNAClean XP beads (Beckman Coulter) with a 2× ratio of beads to reaction volume and an additional supplementation of 1.8× isopropanol and resuspended in nuclease-free water. In vitro transcribed RNA products were then quantified using a NanoDrop One (Thermo Scientific) or on a Take3 plate with absorbance measured by a Cytation 5 (Biotek Instruments). Cas13a was recombinantly expressed and purified as described by Genscript, and was stored in Storage Buffer (600 mM NaCl, 50 mM Tris-HCl pH 7.5, 5% glycerol, 2 mM DTT).

### Nucleic Acid Amplification

Unless specified otherwise, amplification was performed by PCR using Q5 Hot Start polymerase (New England Biolabs) using primer pools (with 150 nM of each primer) in 20 μl reactions. Amplified samples were stored at −20° C. until use. For details about thermal cycling conditions, see Methods.

### Cas13 Detection Reactions

Cas13 detection reactions: Detection assays were performed with 45 nM purified LwaCas13a, 22.5 nM crRNA, 500 nM quenched fluorescent RNA reporter (RNAse Alert v2, Thermo Scientific), 2 μl murine RNase inhibitor (New England Biolabs) in nuclease assay buffer (40 mM Tris-HCl, 60 mM NaCl, pH 7.3) with 1 mM ATP, 1 mM GTP, 1 mM UTP, 1 mM CTP, and 0.6 μl T7 polymerase mix (Lucigen). Input of amplified nucleic acid varied by assay with details described herein. Detection mixes were prepared as 2.2× master mix, such that each droplet contained a 2× master mix after color coding and a 1× master mix after droplet merging.

### Color Coding, Emulsification, and Droplet Pooling

Color coding: Unless specified otherwise, amplified samples were diluted 1:10 into nuclease-free water supplemented with 13.2 mM MgCl₂ prior to color coding to achieve a final concentration of 6 mM after droplet merging. Detection mixes were not diluted. Color code stocks (2 μL) were arrayed in 96 W plates (for detailed information on construction of color codes, see Methods., below). Each amplified sample or detection mix (18 μL) was added to a distinct color code and mixed by pipetting.

Emulsification: The color-coded reagents (20 μL) and 2% 008-fluorosurfactant (RAN Biotechnologies) in fluorous oil (3M 7500, 70 μL) were added to a droplet generator cartridge (Bio Rad), and reagents were emulsified into droplets using a droplet generator (QX200, Bio Rad).

Droplet pooling: A total droplet pool volume of 150 μL of droplets was used to load each standard chip; a total of 800 μL of droplets was used to load each mChip. To maximize the probability of forming productive droplet pairings (amplified sample droplet+detection reagent droplet), half the total droplet pool volume was devoted to target droplets and half to detection reagent droplets. For pooling, individual droplet mixes were arrayed in 96 W plates. A multichannel pipet was used to transfer the requisite volumes of each droplet type into a single row of 8 droplet pools, which were further combined to make a single droplet pool. The final droplet pool was pipetted up and down gently to fully randomize the arrangement of the droplets in the pool.

### Loading, Imaging, and Merging Microwell Arrays

Microwell array loading (standard chips): Loading of standard chips was performed as described previously. Briefly, each chip was placed into an acrylic chip-loader, such that the chip was suspended ˜300-500 μm above the surface of hydrophobic glass, creating a flow space between the chip and the glass. The flow space was filled with fluorous oil (3M, 7500) until loading; immediately before loading, fluorous oil was drained from the flow space. In a single pipetting step, the droplet pool was added to the flow space (FIG. 20, step 3). The loader was tilted to move the droplet pool within the flow space until the microwells were filled with droplets. Fresh fluorous oil (3M 7500) without surfactant was used to wash the flow space (3×1 mL), the flow space was filled with oil, and the chip was sealed against the glass by screwing the loader shut (FIG. 20, step 4). Additional oil (1 mL) was added to the loading slot, and the slot was sealed with clear tape (Scotch) to prevent evaporation.

Microwell array loading (mChips): The back of an mChip was pressed against the lid of the mChip loader to adhere the chip to the lid and leave the microwell array facing out (FIG. 25C, middle illustration). The lid was placed on the loader base, such that opposing magnets in the lid and base held the lid and chip suspended above the base (FIG. 25C, right illustration, and FIG. 25D). Wingnuts on screws were used to push the lid toward the base until the flow space between the surface of the chip and base was ˜300-500 μm (FIG. 25C, right illustration). The flow space was filled with fluorous oil (3M, 7500) until loading; immediately before loading, fluorous oil was drained from the flow space. In a single pipetting step, the droplet pool was added to the flow space by pipetting along the edge of the chip (FIG. 25D, step 3). The loader was tilted to move the droplet pool within the flow space until the microwells were filled with droplets. Fresh fluorous oil (3M 7500) without surfactant was used to wash the flow space (3×1 mL). Two pieces of PCR film (MicroAmp, Applied Biosystems) were joined by placing the sticky side of one piece a few millimeters over the edge of the other piece. The sheet of PCR film was wetted with fluorous oil and set aside. Returning to the loader: the wingnuts were removed so the lid of the loader (with the mChip attached) could be removed from the base. The mChip was sealed against the sheet of wet PCR film in a single smooth motion (FIG. 25D, step 4). The excess PCR film hanging over the edges of the chip was trimmed with a razor blade.

Microwell array imaging, merging, and subsequent imaging: After chip loading, the color code of each droplet was identified by fluorescence microscopy (FIG. 20, step 4). After imaging, the droplet pairs in each microwell were merged by passing the tip of a corona treater over the glass or PCR film (FIG. 20, step 5). The merged droplets were immediately imaged by fluorescence microscopy (FIG. 20, step 6) and placed in an incubator (37° C.) until subsequent imaging time points. All imaging was conducted on a Nikon TI2 microscope equipped with an automated stage (Ludl Electronics, Bio Precision 3 LM), LED light source (Sola), and camera (Hamamatsu). Standard chips were imaged using a 2× objective, while a 1× objective was used for mChips in order to reduce imaging time. During imaging, the microscope condenser was tilted back to reduce background fluorescence in the 488 channel. Additionally, during experiments involving UV channel imaging, black cloth was draped over the microscope to reduce background fluorescence from light scattered off the ceiling.

### Data Analysis

Data analysis: Imaging data were analyzed with custom Python scripts. Analysis consisted of three parts: (1) pre-merge image analysis to determine the identity of the contents of each droplet based on droplet color codes; (2) post-merge image analysis to determine the fluorescence output of each droplet pair and map those fluorescence values back to the contents of the microwell; (3) statistical analysis of the data obtained in parts 1 and 2.

Pre-merge image analysis: The contents of each droplet were determined from images taken before droplet merging: a background image was subtracted from each droplet image, and fluorescence channel intensities were scaled so the intensity range of each channel was approximately the same. Droplets were identified using a Hough transform, and the fluorescence intensity of each channel at each droplet position was determined from a locally convolved image. Compensation for cross-channel optical bleed was applied, and all fluorescence intensities were normalized to the sum of the 647 nm, 594 nm, and 555 nm channels. For 4-channel data sets, analysis of 3-color space was performed directly on normalized intensities. For 5-channel data sets, droplets were divided into UV intensity bins for downstream analysis (FIG. 24). The 3-color space of each UV bin was analyzed separately. The 3-color intensity vectors for each droplet were projected onto the unit simplex, and density-based spatial clustering of applications with noise (DBSCAN) was used to assign labels to each color code cluster. Manual clustering adjustments were made when necessary. For 5-channel data sets, UV intensity bins were recombined after assignments to create the full data set (FIG. 24).

Post-merge image analysis: Background subtraction, intensity scaling, compensation, and normalization were performed as in pre-merge analysis. Following image registration of pre- and post-merge images, the fluorescence intensity of the reporter channel at each droplet pair position was determined from a locally convolved image. The physical mapping of the fluorescent reporter channel onto the previously determined positions of each color code served to assign the fluorescence signal in the reporter channel to the contents of each well. Quality filtering for appropriate post-merge droplet size (which excludes unmerged droplet pairs) and closeness of a droplet's color code to its designated cluster (see FIG. 24) was applied.

Statistical analysis: Heat maps were generated from the median fluorescence value of each crRNA-Target pair. Performance of each guide was assessed by calculating a receiver operating characteristic (ROC) curve for the fluorescence distribution from on-target and all off-target droplets and determining the area under the curve (AUC).

### Experiment-Specific Protocols

### Zika Detection (FIG. 15C)

Nucleic acid amplification: For Zika virus detection (FIG. 15C, FIG. 22), recombinase polymerase amplification (RPA) was used. RPA reactions were performed using the Twist-Dx RT-RPA kit according to the manufacturer's instructions. Primer concentrations were 480 nM and MgAc concentration was 17 mM. For amplification reactions involving RNA, Murine RNase inhibitor (New England Biolabs M3014L) was used at a final concentration of 2 units per microliter. All RPA reactions were incubated at 41° C. for 20 minutes unless otherwise stated. RPA primer sequences are listed. RPA reactions were diluted 1:10 in nuclease-free water prior to color coding.

Cas13 detection reactions: For Zika detection experiments (FIG. 15C), detection mixes were supplemented with MgCl₂ at a final concentration of 6 mM prior to droplet merging. For comparison between CARMEN and SHERLOCK (FIG. 22), a Biotek Cytation 5 plate reader was used for measuring fluorescence of the detection reaction. Fluorescence kinetics were monitored using a monochromator with excitation at 485 nm and emission at 520 nm with a reading every 5 minutes for up to 3 hours.

### Human-Associated Virus Panel (FIG. 16)

Nucleic acid amplification: For the Human-associated viral panel, amplification was performed using Q5 Hot Start polymerase (New England Biolabs) using primer pools (with 150 nM of each primer) in 20 μl reactions. The following thermal cycling conditions were used: (i) initial denaturation at 98° C. for 2 m; (ii) 45 cycles of 98° C. for 15 s, 50° C. for 30 s, and 72° C. for 30 s; (iii) final extension at 72° C. for 2 m.

### Influenza A (FIG. 17)

Seedstock information: Viral seedstocks from three influenza A virus strains were used in this study: A/Puerto Rico/8/1934 (H1N1), A/Hong Kong/1-1-MA-12/1968 (H3N2), and A/Hong Kong/i/1968-2 mouse-adapted 21-2 (H3N2).

Nucleic acid amplification: For the Influenza subtyping panel, amplification was performed using Q5 Hot Start polymerase (New England Biolabs) using primer pools (with 150 nM of each primer) in 20 μl reactions. The following thermal cycling conditions were used: (i) initial denaturation at 98° C. for 2 m; (ii) 40 cycles of 98° C. for 15 s, 52° C. for 30 s, and 72° C. for 30 s; (iii) final extension at 72° C. for 2 m. For the experiments shown in FIGS. 3D, H and N amplification reactions were diluted together. H reactions were diluted 1:10, and N were diluted 1:5, into nuclease-free water supplemented with 13.2 mM MgCl₂ prior to color coding.

### HIV DRMs (FIG. 18)

Nucleic acid amplification: For the HIV DRM panels, amplification was performed using Q5 Hot Start polymerase (New England Biolabs) using primer pools (with 150 nM of each primer) in 20 μl reactions. The following thermal cycling conditions were used: (i) initial denaturation at 98° C. for 2 m; (ii) 40 cycles of 98° C. for 15 s, 52° C. for 30 s, and 72° C. for 30 s; (iii) final extension at 72° C. for 2 m. For the experiments shown in FIG. 4, even and odd reactions were diluted together at 1:10 into nuclease-free water supplemented with 13.2 mM MgCl₂ prior to color coding.

### Software and Nucleic Acid Sequence Design

### Human-Associated Virus Panel Design

Overview: A schematic overview of the human-associated virus panel sequence design strategy is shown in FIG. 26. Briefly, the design pipeline consisted of viral genomes segment alignment, PCR amplicon selection, followed by crRNA selection with cross-reactivity checking. Finally, PCR primers were pooled phylogenetically.

Viral genome segment alignment: Viral genome neighbors were downloaded from NCBI. Each segment of each viral species was aligned using mafft v7.31 with the following parameters: --retree 1 --preservecase. Alignments were curated to remove sequences that were assigned the wrong species, reverse-complemented, or came from the wrong genome segment. A link to the aligned genome segments can be found at:

PCR amplicon selection: Potential PCR binding sites were identified by using CATCH-dx with a window size and length of 20 nucleotides, and a coverage requirement of 90% of the sequences in the alignment. (1) Automated and continuous crRNA design to comprehensively target diverse sequences. Manuscript in preparation. 2) Capturing sequence diversity in metagenomes with comprehensive and scalable probe design. Nature Biotechnology (2019).)

Potential pairs of primer binding sites within a distance of 70 and 200 nucleotides were selected. These sets of potential primer pairs were input into primer3 v2.4.0 to see if suitable PCR primers could be designed for amplification. Primer3 was run using the following parameters: PRIMER_TASK=generic, PRIMER_EXPLAIN_FLAG=1, PRIMER_MIN_SIZE=15, PRIMER_OPT_SIZE=18, PRIMER_MAX_SIZE=20, PRIMER_MIN_GC=30.0, PRIMER_MAX_GC=70.0, PRIMER_MAX_Ns_ACCEPTED=0, PRIMER_MIN_TM=52.0, PRIMER_OPT_TM=54.0, PRIMER_MAX_TM=56.0, PRIMER_MAX_DIFF_TM=1.5, PRIMER_MAX_HAIRPIN_TH=40.0, PRIMER_MAX_SELF_END_TH=40.0, PRIMER_MAX_SELF_ANY_TH=40.0, PRIMER_PRODUCT_SIZE_RANGE=70-200. A list of potential amplicons was generated by parsing the primer3 output file, filtering to ensure that the maximum difference in melting temperature between any pair of forward and reverse primers was less than 4° C. (so that all primers in the pool would have similar PCR efficiency). This list of potential amplicons was then scored based on the average pairwise penalty between all pairs of forward and reverse primers in the design, as measured by primer3. The amplicon with the highest score from each species was chosen for crRNA design.

crRNA design: Software package called CATCH-dx was used to determine the minimum number of crRNAs required to bind to 90% of the sequences within a 40 nt window of each amplicon alignment, allowing for up to one mismatch within the window, and allowing for G-U pairing. These crRNA sets were tested for cross-reactivity at the family level, requiring 3 or more mismatches for >99% of sequences in the other species within the same family, allowing for G-U pairing. This stringent threshold was chosen to ensure high specificity for the human-associated virus assay. For closely related viral genuses (enterovirus, and poxvirus), regions were selected where the majority consensus sequence for each species differed and only considered crRNAs in windows where there was sufficient sequence divergence at the majority consensus level.

Primer pooling: Primers were designed for a set of 169 species that have at least one segment with >=10 sequences in the database, hereafter referred to as the human-associated virus panel 10 version 1 or hav10-v1. Due to limitations of multiplexed PCR, the 210 primer pairs designed for the 169 hav10 species in the version 1 design were split into 15 primer pools, described in more detail below.

Conserved primer pool: 14 species were selected conserved species as a pilot experiment to test the primer design algorithm and pooling strategy. These species were combined into a single “conserved” primer pool at 150 nM final concentration.

TABLE 3
HAV Round 1 Targets and crRNAs

| | | | | crRNA | Synthetic target sequence |
|---|---|---|---|---|---|
| | | | | spacer | |
| | | # | # | sequence | |
| crRNA | Species name | primers | crRNAs | tested | |
| crRNA | ---Deltavirus--- | 3 | 3 | Aggcccucgag | GCCGGCTACTCTTCTTTCCCTTCTCTCGTCTTCCTCG |
| 1 | Hepatitis_delta_virus--- | | | aacaagaagaa | GTCAACCTCCTGAGTTCCTCTTCTTCCTCCTTGCTGA |
| | NA.aligned.p90.p3.3.tsv | | | gcagcu (SEQ | GGCTCTTCCCTCCCGCGGAGAGCTGCTTCTTCTTGT |
| | | | | ID NO: 28) | TCTCGAGGGCCTTCCTTCGTCGGTGA (SEQ ID |
| | | | | | NO: 29) |
| crRNA | Adenoviridae--- | 1 | 1 | Cugcgccuccu | AATGGATTCGGGGGAGTATGCATCCGCACCGCAG |
| 2 | Mastadenovirus--- | | | gcggugcggau | GAGGCGCAGACGGTTTCGCACTCCACGAGCCAGG |
| | Human_mastadenovirus_B | | | gcauac (SEQ | TCAGATCCGGCTCATCGGGGTCAAAAACAAG (SEQ |
| | ---NA.aligned.p90.p3.1.tsv | | | ID NO: 30) | ID NO: 31) |
| crRNA | Adenoviridae--- | 1 | 1 | Gaucggcucgc | GTAGGTGACAAAGAGACGCTCGGTGCGAGGATGC |
| 3 | Mastadenovirus--- | | | auccucgcacc | GAGCCGATCGGGAAGAACTGGATCTCCCGCCACC |
| | Human_mastadenovirus_C- | | | gagcgu (SEQ | AGTTGGAGGAGTGGCTGTTGATGTGGTGAAAGTA |
| | --NA.aligned.p90.p3.1.tsv | | | ID NO: 32) | GAAGTCCCTGCGACGGGCCGAACACTCGTGCTGG |
| | | | | | CTTTTGTAAA (SEQ ID NO: 33) |
| crRNA | Adenoviridae--- | 1 | 1 | Cgcucucguac | GTGCGTTCTCTTCCTTGTTAGAGATGAGGCGCGCG |
| 4 | Mastadenovirus--- | | | gagggaggagg | GTGGTGTCTTCCTCTCCTCCTCCCTCGTACGAGAGC |
| | Human_mastadenovirus_D | | | agagga (SEQ | GTGATGGCGCAGGCGACCCTGGAGGTTCCGTTTGT |
| | ---NA.aligned.p90.p3.1.tsv | | | ID NO: 34) | GCCTCCGCGGTATATGGCTCCTAC (SEQ ID NO: 35) |
| crRNA | Adenoviridae--- | 1 | 1 | Aggagcgcacg | CCTGGCCTACAACTATGGCGACCGCGAGAAGGGC |
| 5 | Mastadenovirus--- | | | cccuucucgcg | GTGCGCTCCTGGACGCTGCTCACCACCTCGGACGT |
| | Human_mastadenovirus_E- | | | gucgcc (SEQ | CACCTGCGGCGTGGAGCAAGTCTACTGGTC (SEQ |
| | --NA.aligned.p90.p3.1.tsv | | | ID NO: 36) | ID NO: 37) |
| crRNA | Adenoviridae--- | 1 | 1 | Cacacaaaaaa | CCAGCGCTTGGATTACATGAAGATCTGTGTTCTTTT |
| 6 | Mastadenovirus--- | | | gaacacagauc | TTGTGTGCTAAGTTTAACAAGTAGCCTAAGGACTT |
| | Human_mastadenovirus_F- | | | uucaug (SEQ | CACCTACAACCGTTGGTTCCTTACGTCAGCTACAAG |
| | --NA.aligned.p90.p3.1.tsv | | | ID NO: 38) | ATTCCACCAAAGGTACACAC (SEQ ID NO: 39) |
| crRNA | Anelloviridae------ | degen | 1 | Ggagauucuc | GCTACAGTAAGATATTACCCCTCACGGAGAAGAAA |
| 7 | Torque_teno_Leptonychotes_ | | | uuucuucucc | GAGAATCTCCGTTCGAGGTTGGGAGC (SEQ ID |
| | weddellii_virus-1---NA | | | gugagggg | NO: 41) |
| | | | | (SEQ ID | |
| | | | | NO: 40) | |
| crRNA | Anelloviridae------ | 1 | 1 | Uuugcuguac | TGAGTTTTTGCTGCTGGAGGACACAGCACACGGA |
| 8 | Torque_teno_Leptonychotes_ | | | ggaucggccgc | GCTCAGTAATTGTGAGTAGCGAAGTGTCTGTGAG |
| | weddellii_virus-2--- | | | ccgauaa | GCCGGGCGGGTGCAGTAGGCCTAAAGCCGAATCA |
| | NA.aligned.p90.p3.1.tsv | | | (SEQ ID | AGGGGCTTATCGGGCGGCCGATCCGTACAGCAAA |
| | | | | NO: 42) | AC (SEQ ID NO: 43) |
| crRNA | Anelloviridae--- | 2 | 1 | Gacuucggug | TGATCTTGGGCGGGAGCCGAAGGTGAGTGAAACC |
| 9 | Betatorquevirus---TTV- | | | guuucacucac | ACCGAAGTCTAGGGGCAATTCGGGCTAGATCAGT |
| | like_mini_virus | | | cuucggc | CTGGCGG (SEQ ID NO: 45) |
| | NA.aligned.p90.p3.2.tsv | | | (SEQ ID | |
| | | | | NO: 44) | |
| crRNA | Anelloviridae---Gyrovirus--- | 1 | 1 | Ccuccucuuaa | ATATGCGCGTAGAAGATCCTTTGATCGCCGCGTTA |
| 10 | Avian_gyrovirus_2--- | | | cgcggcgauca | AGAGGAGGATCTTCAACCCACACCCGGGCTCCTAT |
| | NA.aligned.p90.p3.1.tsv | | | aaggau (SEQ | GTGGTAAGGCTACCGAACCCTTACAATAAGCTTAC |
| | | | | ID NO: 46) | CCTCTTTTTCCAAGGCATTGTATTCATTCCGGAGGC |
| | | | | | (SEQ ID NO: 47) |
| crRNA | Anelloviridae---Gyrovirus--- | 1 | 1 | Accguugaug | TGAACGCTCTCCAAGAAGATACTCCACCCGGACCA |
| 11 | Chicken_anemia_virus--- | | | guccgggugga | TCAACGGTGTTCAGGCCACCAACAAGTTCACGGCC |
| | NA.aligned.p90.p3.1.tsv | | | guaucuu | GTTGGAAACCCCTCACTGCAGAGAGATCCGGATTG |
| | | | | (SEQ ID | GTATCGCTGGAA (SEQ ID NO: 49) |
| | | | | NO: 48) | |
| crRNA | Anelloviridae--- | 1 | 1 | Uuaauucuga | GCTCAAGTCCTCATTTGCATAGGGTGTAACCAATC |
| 12 | lotatorquevirus--- | | | uugguuacacc | AGAATTAAGGCGTTCCCAGTAAAGTGAATATAAGT |
| | Torque_teno_sus_virus_1a | | | cuaugca | AAGTGCAGTTCCGAATGGCTGAGTTT (SEQ ID |
| | ---NA.aligned.p90.p3.1.tsv | | | (SEQ ID | NO: 51) |
| | | | | NO: 50) | |
| crRNA | Anelloviridae--- | 1 | 3 | Gccagaagccc | AAGCTCCGGTCATACAATGGTTCCCTCCTAGCCGG |
| 13 | lotatorquevirus--- | | | ucuaugaggca | AGAACCTGCCTCATAGAGGGCTTCTGGCCGTTGAG |
| | Torque_teno_sus_virus_1b | | | gguucu (SEQ | CTACGGACACTGGTTCCGTAC (SEQ ID NO: 53) |
| | ---NA.aligned.p90.p3.1.tsv | | | ID NO: 52) | |
| crRNA | Arenaviridae---Arenavirus-- | 1 | 1 | Uuaagucuag | GACGTTTGGTGGAGTGATTTTTTCAAACCTAACCTA |
| 14 | - | | | guuagguuug | GACTTAAGATAAGATCTCATCATTGCATTCACAACA |
| | Mopeia_Lassa_virus_reass | | | aaaaaauc | TTGAAAGGTACCTCAATTAACTTGTGAATGTGCCA |
| | ortant_29--- | | | (SEQ ID | CGACAGCAAAGTGGACACGTAA (SEQ ID NO: 55) |
| | L.aligned.p90.p3.1.tsv | | | NO: 54) | |
| crRNA | Arenaviridae--- | 1 | 1 | Gauaugaaaa | ATGAACAGGACAAGTCACCATTGTTAACAGCCATT |
| 15 | Mammarenavirus--- | | | uggcuguuaa | TTCATATCACAGATTGCACGTTCGAATTCCTTTTCT |
| | Argentinian_mammarenavirus | | | caauggug | GAATTCAAGCATGTGTATCTCATTGAACTACCCACA |
| | ---L.aligned.p90.p3.1.tsv | | | (SEQ ID | GCTTCTGAG (SEQ ID NO: 57) |
| | | | | NO: 56) | |
| crRNA | Arenaviridae--- | 1 | 1 | Ugaggaaggu | AATCTGATGAGATGTGGCCTATTCCAACTCATCACC |
| 16 | Mammarenavirus--- | | | gaugaguugg | TTCCTCATTTTGGCTGGCAGAAGTTGTGATGGCAT |
| | Cali_mammarenavirus--- | | | aauaggcc | GATGATTGATAGAAGGCACAATCTCACC (SEQ ID |
| | S.aligned.p90.p3.1.tsv | | | (SEQ ID | NO: 59) |
| | | | | NO: 58) | |
| crRNA | Arenaviridae--- | 2 | 2 | Acuauugaua | CGACACCATTAGCCACACATTGATCACAAATTGTAT |
| 17 | Mammarenavirus--- | | | caauuuguga | CAATAGTTTCAGCAAGTTGTGTTGGAGTTTTACACT |
| | Guanarito_mammarenavirus | | | ucaaugug | TGACATTATGCAATGCTGCAGANACAAACTTGGTT |
| | ---L.aligned.p90.p3.2.tsv | | | (SEQ ID | AACAGAGGTGTTTCCTCACCCATGA (SEQ ID |
| | | | | NO: 60) | NO: 61) |
| crRNA | Arenaviridae--- | 2 | 1 | Ucguccugua | CGCCGAAAGGCGGTGGGTCACGGGGGCGTCCATT |
| 18 | Mammarenavirus--- | | | aauggacgccc | TACAGGACGACCTTGGGGCTTGAGGTTCTAAACAC |
| | Lassa_mammarenavirus--- | | | ccgugac | CATGTCTCTGGGGAGAACTGCTCTCAAAACTGGTA |
| | S.aligned.p90.p3.2.tsv | | | (SEQ ID | TATTGAGTCCTCCTGACACAGCTGCATCATACATTA |
| | | | | NO: 62) | T (SEQ ID NO: 63) |
| crRNA | Arenaviridae--- | 7 | 3 | Uguugacuug | TCATTGCATTCACAACAGGAAAGGGAACTTCAACA |
| 19 | Mammarenavirus--- | | | gcauaugcaua | AGTTTGTGCATGTGCCAAGTTAACAAGGTGCTAAC |
| | Lymphocytic_choriomeningitis_ | | | aacuugu | ATGATCCTTNC (SEQ ID NO: 65) |
| | mammarenavirus--- | | | (SEQ ID | |
| | L.aligned.p90.p3.7.tsv | | | NO: 64) | |
| crRNA | Arenaviridae--- | 1 | 1 | Acaccauugcu | CTGACAATTGTGTGGGTGTTTTACACTTTACATTAT |
| 20 | Mammarenavirus--- | | | cacaaaguuug | GTAAAGCTGCAGCAACAAACTTTGTGAGCAATGGT |
| | Machupo_mammarenavirus | | | uugcug (SEQ | GTTTCTTCACCCATGACA (SEQ ID NO: 67) |
| | ---L.aligned.p90.p3.1.tsv | | | ID NO: 66) | |
| crRNA | Arenaviridae--- | 2 | 2 | Ugucaaguug | GATGCTCAAANCTCTTCCAAACAAGNTCTTCAAAA |
| 21 | Mammarenavirus--- | | | agugcagaaga | ATTCGTGATTCTTCTGCACTCANCTTGACATCAACA |
| | Whitewater_Arroyo_ | | | gucacgg | ATTTTCANATCTTGTCTNCCATGCATATCAAAAAGC |
| | mammarenavirus--- | | | (SEQ ID | TTTCTAATNTCATCTGCACCTTGTGCAGTGAAAACC |
| | S.aligned.p90.p3.2.tsv | | | NO: 68) | ATTGA (SEQ ID NO: 69) |
| crRNA | Astroviridae--- | 1 | 1 | Caguccguga | CTCCATGGGAAGCTCCTATGCTATCAGTTGCTTGCT |
| 22 | Mamastrovirus--- | | | uaggcagugu | GCGTTCATGGCAGAAGATCACCCTTTTAAGGTGTA |
| | Mamastrovirus_1--- | | | ucuacaua | TGTAGAACACTGCCTATCACGGACTGCAAAGCAGC |
| | NA.aligned.p90.p3.1.tsv | | | (SEQ ID | TTCGTGACTCTGG (SEQ ID NO: 71) |
| | | | | NO: 70) | |
| crRNA | Caliciviridae---Norovirus--- | 4 | 1 | Gaucgcccucc | AGCCAATGTTCAGATGGATGAGATTCTCAGATCTG |
| 23 | Norwalk_virus--- | | | cacgugcucag | AGCACGTGGGAGGGCGATCGCAATCTGGCTCCCA |
| | NA.aligned.p90.p3.4.tsv | | | aucuga (SEQ | GTTTTGTGAATGAAGATGGCGTCGAAT (SEQ ID |
| | | | | ID NO: 72) | NO: 73) |
| crRNA | Caliciviridae---Sapovirus--- | degen | 1 | Agucaucacca | GGGCTCCCATCTGGCATGCCATTCACCA |
| 24 | Sapporo_virus---NA | | | uaggugugga | GTGTCATCAATTCWGTCAACCACATGAT |
| | | | | cagucuc | ATACTTTGCCGCGGCTGTGCTGCAGGCC |
| | | | | (SEQ ID | TATGAGGAACACAATGTGCCATACACTG |
| | | | | NO: 74) | GCAATGTGTTCCAGATTGAGACTGTCCA |
| | | | | | CACCTATGGTGATGACTGCATGTA (SEQ |
| | | | | | ID NO: 75) |
| crRNA | Coronaviridae--- | 1 | 1 | Augggcacaa | TAGTGTCAAACGTGATGGTGTGCAAGTTGGTTATT |
| 25 | Alphacoronavirus--- | | | uaaccaacuug | GTGCCCATGGTATTAAGTACTATTCACGTGTTAGA |
| | Human_coronavirus_229E- | | | cacacca (SEQ | AGTGTTAGCGGTAGAGCTA (SEQ ID NO: 77) |
| | --NA.aligned.p90.p3.1.tsv | | | ID NO: 76) | |
| crRNA | Coronaviridae--- | 1 | 1 | Aauggugaac | GTGGTGAATGGAATGCTGTGTATAGGGCGTTTGG |
| 26 | Alphacoronavirus--- | | | caaacgcccua | TTCACCATTTATTACAAATGGTATGTCATTGCTAGA |
| | Human_coronavirus_NL63- | | | uacacag | TATAATTGTTAAACCAGTTTTCTTTAATGCTTTTGTT |
| | --NA.aligned.p90.p3.1.tsv | | | (SEQ ID | AAATGCAATTGTGGTTCTGAGAGTTGGAGTGTTGG |
| | | | | NO: 78) | TG (SEQ ID NO: 79) |
| crRNA | Coronaviridae--- | 1 | 1 | Gcuugaccag | TGAAGTCAGATGAGGGTGGGTTATGCCCCTCTACT |
| 27 | Betacoronavirus--- | | | uagaggggcau | GGTCAAGCGATGGAAAGTGTTGGATTCGTTTATGA |
| | Human_coronavirus_HKU1 | | | aacccac (SEQ | TAATCATGTGAAGATAGATTGTCGCTGCATTCTTG |
| | ---NA.aligned.p90.p3.1.tsv | | | ID NO: 80) | GACAAGAATGGCATGT (SEQ ID NO: 81) |
| crRNA | Coronaviridae--- | 1 | 1 | Gcuuccugau | CCTTTGCTGAGTTGGAAGCTGCGCAGAAAGCCTAT |
| 28 | Betacoronavirus--- | | | aggcuuucugc | CAGGAAGCTATGGACTCTGGTGACACCTCACCACA |
| | Middle_East_respiratory_ | | | gcagcuu | AGTTCT (SEQ ID NO: 83) |
| | syndrome- | | | (SEQ ID | |
| | related_coronavirus--- | | | NO: 82) | |
| | NA.aligned.p90.p3.1.tsv | | | | |
| crRNA | Coronaviridae--- | 1 | 1 | Uguccucaccu | TGTCTGCATGTTGTTGGACCTAACCTAAATGCAGG |
| 29 | Betacoronavirus--- | | | gcauuuaggu | TGAGGACATCCAGCTTCTTAAGGCAGCATATGAAA |
| | Severe_acute_respiratory_ | | | uaggucc | ATTTCAATTCACAGGACATCTTACTTGCACCATTGT |
| | syndrome- | | | (SEQ ID | TGTCAGCAG (SEQ ID NO: 85) |
| | related_coronavirus--- | | | NO: 84) | |
| | NA.aligned.p90.p3.1.tsv | | | | |
| crRNA | Filoviridae---Ebolavirus--- | 1 | 1 | Gacaauuagg | TAATTCAGTTGCTCAGGCTCGCTTTTCAGGACTCCT |
| 30 | Reston_ebolavirus--- | | | aguccugaaaa | AATTGTCAAAACCGTTCTTGATCATATTCTGCAAAA |
| | NA.aligned.p90.p3.1.tsv | | | gcgagcc | AACCGACCAAGGAGTAAGAC (SEQ ID NO: 87) |
| | | | | (SEQ ID | |
| | | | | NO: 86) | |
| crRNA | Filoviridae---Ebolavirus--- | 1 | 1 | Cuuugcaacac | TAGTCAATCCCCCATTTGGGGGCATTCCTAAAGTG |
| 31 | Sudan_ebolavirus--- | | | uuuaggaaug | TTGCAAAGGTATGTGGGTCGTATTGCTTTGCCTTTT |
| | NA.aligned.p90.p3.1.tsv | | | cccccaa (SEQ | CCTAACCTGG (SEQ ID NO: 89) |
| | | | | ID NO: 88) | |
| crRNA | Filoviridae---Ebolavirus--- | 1 | 1 | Ugacuguuuu | TGCCTAACAGATCGACCAAGGGTGGACAACAGAA |
| 32 | Zaire_ebolavirus--- | | | ucuguugucc | AAACAGTCAAAAGGGCCAGCATACAGAGGGCAGA |
| | NA.aligned.p90.p3.1.tsv | | | acccuugg | CAGA (SEQ ID NO: 91) |
| | | | | (SEQ ID | |
| | | | | NO: 90) | |
| crRNA | Filoviridae---Marburgvirus-- | 1 | 1 | Ggcuugucuu | CTTCATCAACTGAGGGTCGAAAAAGTCCCAGAGAA |
| 33 | -Marburg_marburgvirus--- | | | cucugggacuu | GACAAGCCTGTTTAGGATTTCGCTTCCTGCCGACAT |
| | NA.aligned.p90.p3.1.tsv | | | uuucgac | GTTCTCAGTA (SEQ ID NO: 93) |
| | | | | (SEQ ID | |
| | | | | NO: 92) | |
| crRNA | Flaviviridae---Flavivirus--- | 1 | 1 | Ugucauugau | TTCTGGATCTGATGGACCATGTCGCATACCCATATC |
| 34 | Bagaza_virus--- | | | auggguaugc | AATGACAGCCAACCTTCAGGATTTGACCCCGATAG |
| | NA.aligned.p90.p3.1.tsv | | | gacauggu | GAAGGCTCATAACGGTCAATCCATATGTGTCTACA |
| | | | | (SEQ ID | TCATCATCGGGGACAAA (SEQ ID NO: 95) |
| | | | | NO: 94) | |
| crRNA | Flaviviridae---Flavivirus--- | 1 | 1 | Gggaacagcac | AGCTGTGGGAATCGACATACCTCCTCGACCACGTG |
| 35 | Culex_flavivirus--- | | | guggucgagga | CTGTTCCCGATGTACGTGATGTTGGCGTTCAATCT |
| | NA.aligned.p90.p3.1.tsv | | | gguaug (SEQ | GAAATCACAGTTCGTACCTGTGGACTCGATGGTAC |
| | | | | ID NO: 96) | TGCTGAACT (SEQ ID NO: 97) |
| crRNA | Flaviviridae---Flavivirus--- | 2 | 1 | Uugacacgcgg | CCGTCTTTCAATATGCTGAAACGCGCGAGAAACCG |
| 36 | Dengue_virus--- | | | uuucucgcgcg | CGTGTCAACTGTTTCACAGTTGGCGAAGAGATTCT |
| | NA.aligned.p90.p3.2.tsv | | | uuucag (SEQ | CA (SEQ ID NO: 99) |
| | | | | ID NO: 98) | |
| crRNA | Flaviviridae---Flavivirus--- | 1 | 1 | Uguuccauuc | GTGTGAAAGAAGACCGCATAGCTTACGGAGGCCC |
| 37 | Japanese_encephalitis_virus | | | cauuuucggu | ATGGAGGTTTGACCGAAAATGGAATGGAACAGAT |
| | ---NA.aligned.p90.p3.1.tsv | | | caaaccuc | GACGTGCAAGTGATCGTGGTAGAACCGGGGAAGG |
| | | | | (SEQ ID | CTGCAGTAAACATCCAGACAAAACCAGGAGT (SEQ |
| | | | | NO: 100) | ID NO: 101) |
| crRNA | Flaviviridae---Flavivirus--- | 1 | 1 | Cuuuaagccac | TTCCAGTGCATGCTCATAGTGATCTTACCGGAAGA |
| 38 | Kyasanur_Forest_disease_ | | | uuaugcccucu | GGGCATAAGTGGCTTAAAGGGGACTCAGTCAAGA |
| | virus--- | | | uccggu (SEQ | CGCATCTGACACGTGTGGAAGGCTGGGTATGGAA |
| | NA.aligned.p90.p3.1.tsv | | | ID NO: 102) | GAATAAGCTCCTGACGATGGCCTTTTGTGCAGTTG |
| | | | | | TGTGG (SEQ ID NO: 103) |
| crRNA | Flaviviridae---Flavivirus--- | 1 | 1 | Cacuaauggg | CAATATGCTAAAACGCGGCATACCCCGCGTATTCC |
| 39 | Murray_Valley_encephalitis_ | | | aauacgcgggg | CATTAGTGGGAGTGAAGAGGGTAGTAATGAACTT |
| | virus--- | | | uaugccg | GCTAGATGGCAGAGGGCCAATACGGTTTGTGTTG |
| | NA.aligned.p90.p3.1.tsv | | | (SEQ ID | GCTCTCTTAGCTTTCTTCAGGTTTACAGCACTTGC |
| | | | | NO: 104) | (SEQ ID NO: 105) |
| crRNA | Flaviviridae---Flavivirus--- | 1 | 1 | Cuccaucaacc | GTTGGGGCAAGTCAATCTTGTGGAGTGTGCCTGAA |
| 40 | Powassan_virus--- | | | cccaucaucau | AGTCCTAGGCGCATGATGATGGGGGTTGATGGAG |
| | NA.aligned.p90.p3.1.tsv | | | gcgccu (SEQ | CTGGGGAGTGCCCCCTGCACAAGAGAGCAACAGG |
| | | | | ID NO: 106) | AGTGTT (SEQ ID NO: 107) |
| crRNA | Flaviviridae---Flavivirus--- | 1 | 1 | Ccacggccauc | CGGGGTTGAAGAGGATACTTGGAAGTCTGCTGGA |
| 41 | Saint_Louis_encephalitis_ | | | cagcagacuuc | TGGCCGTGGACCCGTGCGGTTCATACTAGCCATTC |
| | virus--- | | | caagua (SEQ | TGACATTCTTCCGATTTACAGCTCTACAGCCAACTG |
| | NA.aligned.p90.p3.1.tsv | | | ID NO: 108) | AGGCGCTGAAGCGCAGATGGAGGGCTGTAGAT |
| | | | | | (SEQ ID NO: 109) |
| crRNA | Flaviviridae---Flavivirus--- | 1 | 1 | Cuuccagaacg | GAGGGAGTGAATGGTGTTGAGTGGATCGATGTCG |
| 42 | Tembusu_virus--- | | | acaucgaucca | TTCTGGAAGGAGGCTCATGTGTGACCATCACGGCA |
| | NA.aligned.p90.p3.1.tsv | | | cucaac (SEQ | AAAGACAGGCCGACCATAGACGTCAAGATGATGA |
| | | | | ID NO: 110) | ACATGGAGGCTACGGAATT (SEQ ID NO: 111) |
| crRNA | Flaviviridae---Flavivirus--- | 1 | 1 | Gagggggaccg | GAGAACAAGAGCTGGGGATGGCCAGGAAGGCCA |
| 43 | Tick- | | | ccccccuuucc | TTCTGAAAGGAAAGGGGGGCGGTCCCCCTCGACG |
| | borne_encephalitis_virus--- | | | uuucag (SEQ | AGTGTCGAAAGAGACCG (SEQ ID NO: 113) |
| | NA.aligned.p90.p3.1.tsv | | | ID NO: 112) | |
| crRNA | Flaviviridae---Flavivirus--- | 1 | 1 | Uuaggauugu | CTGTCTCCAACTGTCCAACAACTGGGGAGGCCCAC |
| 44 | Usutu_virus--- | | | gggccucccca | AATCCTAAGAGAGCTGAGGACACGTACGTGTGCA |
| | NA.aligned.p90.p3.1.tsv | | | guuguug | AAAGTGGTGTCACTGACAGGGGCTGGGGCAATGG |
| | | | | (SEQ ID | CTGTGGACTATTTGGCAAAGGAAGTATAGACACGT |
| | | | | NO: 114) | GTGCCA (SEQ ID NO: 115) |
| crRNA | Flaviviridae---Flavivirus--- | 1 | 1 | Gagggugguu | CAAGTCTGGAAGCAGCATTGGCAAAGCCTTTACAA |
| 45 | West_Nile_virus--- | | | guaaaggcuu | CCACCCTCAAAGGAGCGCAGAGACTAGCCGCTCTA |
| | NA.aligned.p90.p3.1.tsv | | | ugccaaug | GGAGACACAGCTTGG (SEQ ID NO: 117) |
| | | | | (SEQ ID | |
| | | | | NO: 116) | |
| crRNA | Flaviviridae---Flavivirus--- | 1 | 1 | Uccaaaugug | ATTGGTCTGCAAATCGAGTTGCTAGGCAATAAACA |
| 46 | Yellow_fever_virus--- | | | uuuauugccu | CATTTGGATTAATTTTAATCGTTCGTTGAGCGATTA |
| | NA.aligned.p90.p3.1.tsv | | | agcaacuc | GCAGAGAACTGACCAGAACATGTCTGGTCGTAAA |
| | | | | (SEQ ID | GCTCAGGGAAAAACCCTGGGCGTCAATATGGTAC |
| | | | | NO: 118) | (SEQ ID NO: 119) |
| crRNA | Flaviviridae---Flavivirus--- | 1 | 1 | Gaccaaguau | AAAAACCCCATGTGGAGAGGTCCACAGAGATTGC |
| 47 | Zika_virus--- | | | augacuuuuu | CCGTGCCTGTGAACGAGCTGCCCCACGGCTGGAA |
| | NA.aligned.p90.p3.1.tsv | | | ggcucguu | GGCTTGGGGGAAATCGTACTTCGTCAGAGCAGCA |
| | | | | (SEQ ID | AAGACAAATAACAGCTTTGTCGTGGATGGTGACAC |
| | | | | NO: 120) | ACTGAAGGAA (SEQ ID NO: 121) |
| crRNA | Flaviviridae---Hepacivirus--- | 2 | 2 | Ugacguccug | TGAGCACAAATCCTAAACCTCAAAGAAAAACCAAA |
| 48 | Hepacivirus_C--- | | | ugggcggcggu | AGAAACACCAACCGTCGCCCACAGGACGTCAAGTT |
| | NA.aligned.p90.p3.2.tsv | | | ugguguu | CCCGGGTGGCGGTCAGATCGTTGGTGGAGTTTACT |
| | | | | (SEQ ID | TGTTGCCGCGCAGGGG (SEQ ID NO: 123) |
| | | | | NO: 122) | |
| crRNA | Flaviviridae---Pegivirus--- | 2 | 1 | Ucagcugcgac | GGTACGGGTTGGAGCCTGACCTGGCTGCGTCTTTG |
| 49 | Pegivirus_A--- | | | ggcugcggugu | CTAAGACTATACGACGACTGCCCCTACACCGCAGC |
| | NA.aligned.p90.p3.2.tsv | | | aggggc (SEQ | CGTCGCAGCTGACATTGGTGAAGCCTCT (SEQ ID |
| | | | | ID NO: 124) | NO: 125) |
| crRNA | Flaviviridae---Pegivirus--- | 2 | 1 | Guguuucccg | ATGTCAGCTGGGCAAAAGTACGCGGCGTCAACTG |
| 50 | Pegivirus_C--- | | | gcacaucgucc | GCCCCTCCTGGTGGGTGTTCAGCGGACGATGTGCC |
| | NA.aligned.p90.p3.2.tsv | | | gcugaac | GGGAAACACTGTCTCCCGGNCCATCGGATGACCCC |
| | | | | (SEQ ID | CAATGGGC (SEQ ID NO: 127) |
| | | | | NO: 126) | |
| crRNA | Flaviviridae---Pegivirus--- | 1 | 1 | Caccacagcga | GGTGGCCATCAAGCTATCTCGAGGCCTGTTATTCG |
| 51 | Pegivirus_H--- | | | auaacaggccu | CTGTGGTGTTGGCGCACGGAGTGTGCCGACCTGG |
| | NA.aligned.p90.p3.1.tsv | | | cgagau (SEQ | GCGGGTATTTGGTCTTGAGGTTTGCGCGGACATCT |
| | | | | ID NO: 128) | CTTGGTTGGTGGAGTTT (SEQ ID NO: 129) |
| crRNA | Hantaviridae--- | 1 | 1 | Caucaggcuca | CTGGCTACAAAACCAGTTGATCCAACAGGGCTTGA |
| 52 | Orthohantavirus--- | | | agcccuguugg | GCCTGATGACCATCTGAAGGAGAAATCATCTCTGA |
| | Andes_orthohantavirus--- | | | aucaac (SEQ | GATATGGGAATGTCCTGGATGT (SEQ ID NO: 131) |
| | S.aligned.p90.p3.1.tsv | | | ID NO: 130) | |
| crRNA | Hantaviridae--- | 1 | 1 | Uagucuauac | CCTTTCCAGTTGGGTCACTGACAGCAGTAGAGTGT |
| 53 | Orthohantavirus--- | | | acucuacugcu | ATAGACTACCTGGATCGTCTCTATGCAATAAGGCA |
| | Dobrava- | | | gucagug | TGACATTGTTGACCAGATGATAAAGCATGACTGGT |
| | Belgrade_orthohantavirus-- | | | (SEQ ID | CAGA (SEQ ID NO: 133) |
| | -L.aligned.p90.p3.1.tsv | | | NO: 132) | |
| crRNA | Hantaviridae--- | 1 | 1 | uauacuggaca | ACACAATGGCCCAGTAGAAGAAATGATGGTGTTG |
| 54 | Orthohantavirus--- | | | acaccaucauu | TCCAGTATATGAGGCTAGTTCAAGCTGAGATAAGT |
| | Hantaan_orthohantavirus-- | | | ucuucu (SEQ | TATGTTAGAGAGCACTTGATCAAAACTGAGGAGA |
| | -L.aligned.p90.p3.1.tsv | | | ID NO: 134) | GAGCTGCACTAGAAGCCATGT (SEQ ID NO: 135) |
| crRNA | Hantaviridae--- | 1 | 1 | Ugaaucuagc | AGGCACAATAGGAGCAGTAGAATGTATCAATTTGC |
| 55 | Orthohantavirus--- | | | aaauugauac | TAGATTCGCTGTATATGGTCCGCCATGACCTAATTG |
| | Imjin_orthohantavirus--- | | | auucuacu | A (SEQ ID NO: 137) |
| | L.aligned.p90.p3.1.tsv | | | (SEQ ID | |
| | | | | NO: 136) | |
| crRNA | Hantaviridae--- | 2 | 1 | Ucugccaugu | TAGAGCACTAATCACAGCATCAGCACTACCACAAC |
| 56 | Orthohantavirus--- | | | ugugguagug | ATGGCAGATATAGAGAGGCTAATAGCGGAGGGCC |
| | Nova_orthohantavirus--- | | | cugaugcu | TTGAAATAGAAAAGGAGCTTATGACAGCTCGTATT |
| | S.aligned.p90.p3.2.tsv | | | (SEQ ID | CGTTTACAGGAGGCAAAGGAGGCTGCAGA (SEQ |
| | | | | NO: 138) | ID NO: 139) |
| crRNA | Hantaviridae--- | 1 | 1 | Cuggcaacaac | AAGAGGATATAACCCGCCATGAACAACAACTTGTT |
| 57 | Orthohantavirus--- | | | aaguuguugu | GTTGCCAGACAAAAACTTAAGGATGCAGAGAGAG |
| | Puumala_orthohantavirus-- | | | ucauggc | CAGTGGAAATGGACCCAGATGACGTTAACAAAAA |
| | -S.aligned.p90.p3.1.tsv | | | (SEQ ID | CACACTGCAAGCAAGGCAACAAACAGTGTCAGC |
| | | | | NO: 140) | (SEQ ID NO: 141) |
| crRNA | Hantaviridae--- | 1 | 1 | Uacuuauuua | TCACAAAGTCTCAGGTGGTTGCTAATAGTATCTTA |
| 58 | Orthohantavirus--- | | | agauacuauu | AATAAGTATTGGGAAGAGCCATATTTTAGCCAAAC |
| | Seoul_orthohantavirus--- | | | agcaacca | AAGGAATATTAGTTTAAAAGGTATGTCAGGCCAAG |
| | L.aligned.p90.p3.1.tsv | | | (SEQ ID | TACAAG (SEQ ID NO: 143) |
| | | | | NO: 142) | |
| crRNA | Hantaviridae--- | 1 | 1 | Cccgaguuug | CACATTACAGAGCAGACGGGCAGCTGTGTCTGCAT |
| 59 | Orthohantavirus--- | | | guuuccaaugc | TGGAGACCAAACTCGGAGAACTCAAACGGGAGCT |
| | Sin_Nombre_orthohantavirus | | | agacaca | GGCTGATCTTATTGCAGCTCAGAAATTGGCTTCAA |
| | ---S.aligned.p90.p3.1.tsv | | | (SEQ ID | AACCTGTTGATCCAACAGGGATTGAACCT (SEQ ID |
| | | | | NO: 144) | NO: 145) |
| crRNA | Hantaviridae--- | 1 | 1 | Uaguuuuuga | CAACCAAACTGAGAAGGCATTAACAGAATCCTCTC |
| 60 | Orthohantavirus--- | | | gaggauucug | AAAAACTNATTCAGGAGATCGACCAGGCTGGACA |
| | Thottapalayam_orthohanta | | | uuaaugcc | AAATCCGGATTCCATTCAGCAGCAGTCTA (SEQ ID |
| | virus--- | | | (SEQ ID | NO: 147) |
| | S.aligned.p90.p3.1.tsv | | | NO: 146) | |
| crRNA | Hantaviridae--- | 1 | 1 | Auuuguccuc | CCGACCCGGATGATGTTAACAAGAGTACACTACAG |
| 61 | Orthohantavirus--- | | | caaugcugaca | AGCAGACGGGCAGCTGTGTCAGCATTGGAGGACA |
| | Tula_orthohantavirus--- | | | cagcugc | AACTGGCAGACTTCAAGAGACAGCTTGCAGATCTG |
| | S.aligned.p90.p3.1.tsv | | | (SEQ ID | GTATCAAGTCAAAAAATGGGTGAAAAGCCTGT |
| | | | | NO: 148) | (SEQ ID NO: 149) |
| crRNA | Hepadnaviridae--- | 1 | 1 | Acggacugagg | GCACCTGTATTCCCATCCCATCATCCTGGGCTTTCG |
| 62 | Orthohepadnavirus--- | | | cccacucccau | CAAAATTCCTATGGGAGTGGGCCTCAGTCCGTTTC |
| | Hepatitis_B_virus--- | | | aggaau (SEQ | TCCTGGCTCAGTT (SEQ ID NO: 151) |
| | NA.aligned.p90.p3.1.tsv | | | ID NO: 150) | |
| crRNA | Hepeviridae--- | 1 | 2 | Ccacgacggcg | TGCCTATGCTGCCCGCGCCACCGGCCGGTCAGCCG |
| 63 | Orthohepevirus--- | | | gccagacggcu | TCTGGCCGCCGTCGTGGGCGGCGCAGCGGCGGTG |
| | Orthohepevirus_A--- | | | ggccgg (SEQ | CCGGCGGTGGTTTCTGGGGTGACAGGGTTGATTCT |
| | NA.aligned.p90.p3.1.tsv | | | ID NO: 152) | CAGCCCTTCGC (SEQ ID NO: 153) |
| crRNA | Herpesviridae--- | 1 | 1 | Auauucucgu | TAAGAGGTTTCAAGTGCGAATCTCAAAGTTCTCAC |
| 64 | Cytomegalovirus--- | | | gagaacuuug | GAGAATATTGTCTTCAAGAATCGACAACTGTGGTC |
| | Human_betaherpesvirus_5 | | | agauucgc | CAAGA (SEQ ID NO: 155) |
| | ---NA.aligned.p90.p3.1.tsv | | | (SEQ ID | |
| | | | | NO: 154) | |
| crRNA | Herpesviridae--- | 1 | 1 | Gaagacggcag | GTGTCTGTGGTTGTCTTCCCAGACTCTGCTTTCTGC |
| 65 | Lymphocryptovirus--- | | | aaagcagaguc | CGTCTTCGGTCAAGTACCAGCTGGTGGTCCGCATG |
| | Human_gammaherpesvirus_ | | | ugggaa (SEQ | TTTTGATCCAAACTTTAGTTTTAGGATTTATGCATC |
| | 4--- | | | ID NO: 156) | CATTATCCCGCAGTTCCA (SEQ ID NO: 157) |
| | NA.aligned.p90.p3.1.tsv | | | | |
| crRNA | Herpesviridae--- | 1 | 1 | Cacgauuggcc | AGCCATTATACACACGGGTTTTTTGTTGTCTTGGCC |
| 66 | Rhadinovirus--- | | | aagacaacaaa | AATCGTGTCTCCATGGCGCTAAAGGGACCACAAAC |
| | Human_gammaherpesvirus_ | | | aaaccc (SEQ | CCTCGAGGAAAATATTGGGTCTGCGGCCCCCACTG |
| | 8--- | | | ID NO: 158) | GTCCCTGCGGGTACCTCTATGCCTATCTGACACACA |
| | NA.aligned.p90.p3.1.tsv | | | | ACTTCCC (SEQ ID NO: 159) |
| crRNA | Herpesviridae--- | 1 | 1 | Gcgccgcuagc | ACGTACACAAACTCGAACGCGGCCACGAAGATGC |
| 67 | Simplexvirus--- | | | aucuucguggc | TAGCGGCGCAGTGGGGCGCCCCCAGGCATTTGGC |
| | Human_alphaherpesvirus_ | | | cgcguu (SEQ | ACAGAGAAACGCGTAATCGGCCACCCACTGGGGC |
| | 1---NA.aligned.p90.p3.1.tsv | | | ID NO: 160) | GAGAGGCGGTAGGTTTGCTTGTACAGCTCGATGG |
| | | | | | T (SEQ ID NO: 161) |
| crRNA | Herpesviridae--- | 1 | 1 | Uggaaacguu | GTGAAAAAGGCAGAGACGTCTCCCGTGGTCGCGA |
| 68 | Simplexvirus--- | | | cgcgaccacgg | ACGTTTCCAGGTGGCCCAGGAGCCGCTCCCCCTCG |
| | Human_alphaherpesvirus_ | | | gagacgu | CGCCACGCGTACTCCAGGAGCAACTC (SEQ ID |
| | 2---NA.aligned.p90.p3.1.tsv | | | (SEQ ID | NO: 163) |
| | | | | NO: 162) | |
| crRNA | Herpesviridae--- | 1 | 1 | Aguagagcuu | ATCCTTGGTTGGTTTTGGTCTAACATAAGATATAAG |
| 69 | Varicellovirus--- | | | auaucuuaug | CTCTACTATAGCGAGCGTGCATACAACAACCCAGG |
| | Human_alphaherpesvirus_ | | | uuagacca | CCAGAATCCGAATGTA (SEQ ID NO: 165) |
| | 3---NA.aligned.p90.p3.1.tsv | | | (SEQ ID | |
| | | | | NO: 164) | |
| crRNA | Nairoviridae--- | 1 | 1 | Gagggaacau | CCTGAATCTGTGGAGGCAGTGCCGGTGACAGAAA |
| 70 | Orthonairovirus---Crimean- | | | uuuucuuucu | GAAAGATGTTCCCTCTGCCTGAGACTCCACTGAGT |
| | Congo_hemorrhagic_fever_ | | | gucaccgg | GAGGTGCATTCAATAGAGCG (SEQ ID NO: 167) |
| | orthonairovirus--- | | | (SEQ ID | |
| | L.aligned.p90.p3.1.tsv | | | NO: 166) | |
| crRNA | Nairoviridae--- | 1 | 1 | Gggcuccuug | CCCTTGAACTAGCCAAGCAGTCAAGTGCCATGAGA |
| 71 | Orthonairovirus--- | | | agcucucaugg | GCTCAAGGAGCCCAGATTGACACTGTTTTTAGCAG |
| | Nairobi_sheep_disease_ | | | cacuuga | CTACTACTGGCTTTGGAAGGCAGGTGTGACTGCAG |
| | orthonairovirus--- | | | (SEQ ID | AGATGTTCCCGACAGTCTCACAGTTTCT (SEQ ID |
| | S.aligned.p90.p3.1.tsv | | | NO: 168) | NO: 169) |
| crRNA | Orthomyxoviridae--- | 1 | 1 | Uuauggccau | TCTAATGTCGCAGTCTCGCACTCGCGAGATACTGA |
| 72 | Alphainfluenzavirus--- | | | augguccacug | CAAAAACCACAGTGGACCATATGGCCATAATTAAG |
| | Influenza_A_virus--- | | | ugguuuu | AAGTACACATCGGGGAGACAGGAAAAGAACCCGT |
| | 1.aligned.p90.p3.1.tsv | | | (SEQ ID | CACTTAGGATGAAATGGATGATGGCAATGA (SEQ |
| | | | | NO: 170) | ID NO: 171) |
| crRNA | Orthomyxoviridae--- | 1 | 1 | Gggaacaccgg | ACAGGCAGCAATTTCAACAACATTCCCATACACCG |
| 73 | Betainfluenzavirus--- | | | uguaugggaa | GTGTTCCCCCTTATTCCCATGGAACGGGAACAGGC |
| | Influenza_B_virus--- | | | uguuguu | TACACAATAGACACCGTGATCAGAAC (SEQ ID |
| | 1.aligned.p90.p3.1.tsv | | | (SEQ ID | NO: 173) |
| | | | | NO: 172) | |
| crRNA | Orthomyxoviridae--- | 1 | 1 | Guagcauggg | ATCTGCTTTAGGAGGACCATTAGGGAAAACTCTAT |
| 74 | Gammainfluenzavirus--- | | | gccaaaagaua | CTTTTGGCCCCATGCTACTCAAGAAAATTTCTGGTT |
| | Influenza_C_virus--- | | | gaguuuu | CCGGAGTAAAAGTTAAAGATACAGTATATATCCAA |
| | 1.aligned.p90.p3.1.tsv | | | (SEQ ID | GGTGTCAGAGCAGTACAA (SEQ ID NO: 175) |
| | | | | NO: 174) | |
| crRNA | Papillomaviridae--- | 1 | 1 | Cucuggcguu | CAGTGGGTATGGCAATACGCAGATGGTTGTTGGA |
| 75 | Alphapapillomavirus--- | | | ccaacaaccau | ACGCCAGAGGAGGTAACGGGGGATGAGNANAGC |
| | Alphapapillomavirus_4--- | | | cugcgua | CAAGGGGGGCGGCCGGTGGAGGATNAGGAGGAG |
| | NA.aligned.p90.p3.1.tsv | | | (SEQ ID | GAGCGTCAAGGGGGAGACGGAGAGGCAGATCTA |
| | | | | NO: 176) | AC (SEQ ID NO: 177) |
| crRNA | Papillomaviridae--- | 2 | 2 | Aaggguuucc | TCCAGATTAGATTTGCACGAGGAAGAGGAAGATG |
| 76 | Alphapapillomavirus--- | | | uucggugucu | CAGACACCGAAGGAAACCCTTTCGGAACGTTTAAG |
| | Alphapapillomavirus_7--- | | | gcaucuuc | TGCGTT (SEQ ID NO: 179) |
| | NA.aligned.p90.p3.2.tsv | | | (SEQ ID | |
| | | | | NO: 178) | |
| crRNA | Papillomaviridae--- | 1 | 1 | Cgcauguguu | GTACAGACCTACGTGACCATATAGACTATTGGAAA |
| 77 | Alphapapillomavirus--- | | | uccaauagucu | CACATGCGCCTAGAATGTGCTATTTATTACAAGGC |
| | Alphapapillomavirus_9--- | | | auauggu | CAGAGAAATGGGATT (SEQ ID NO: 181) |
| | NA.aligned.p90.p3.1.tsv | | | (SEQ ID | |
| | | | | NO: 180) | |
| crRNA | Papillomaviridae--- | 3 | 1 | Ccaaagccuuu | TGAACTTACTGACCAAAGCTGGAAATCTTTTTTTAA |
| 78 | Betapapillomavirus--- | | | uaaaaaaaga | AAGGCTTTGGAAACAATTAGAGCTGAGTGACCAA |
| | Betapapillomavirus_1--- | | | uuuccag | GAAGACGAGGGCGAGGATGGAGAATCTCAGCGA |
| | NA.aligned.p90.p3.3.tsv | | | (SEQ ID | GCGTTTCAATG (SEQ ID NO: 183) |
| | | | | NO: 182) | |
| crRNA | Papillomaviridae--- | 6 | 3 | Cuuguagugc | TAAAAGGCTTTGGACACAATTAGAGCTCAGTGATC |
| 79 | Betapapillomavirus--- | | | auugaaacgu | AAGAAGACGAGGGAGAGGATGGAAACACTCAGC |
| | Betapapillomavirus_2--- | | | ucgcugag | GAACGTTTCAATGCACTGCAAGA (SEQ ID NO: 185) |
| | NA.aligned.p90.p3.6.tsv | | | (SEQ ID | |
| | | | | NO: 184) | |
| crRNA | Paramyxoviridae--- | 2 | 1 | Aggugcagga | GAGTCACAACCATCAGCTGGTGCAACCCCTCATGC |
| 80 | Avulavirus--- | | | guauugucuu | GCTCCAGTCAGGGCAGAGCCAAGACAATACTCCTG |
| | Avian_avulavirus_1--- | | | ggcucugc | TACCTGTGGATCATGTCCAGCTACCTGTCGACTTTG |
| | NA.aligned.p90.p3.2.tsv | | | (SEQ ID | TGCAGGCGATGATGTCTATGATGGAGGCATTATCA |
| | | | | NO: 186) | CA (SEQ ID NO: 187) |
| crRNA | Paramyxoviridae--- | 1 | 1 | Ugaggcgagca | AAAGGAACTCCAACACCAGGTCCGGACTCAATCCT |
| 81 | Avulavirus--- | | | aggauugagu | TGCTCGCCTCAGACAAGCAAACTGAGAGGTTCATC |
| | Avian_avulavirus_4--- | | | ccggauc | TTCCTCAACACTTACGGGTTTATCTATGACACTACA |
| | NA.aligned.p90.p3.1.tsv | | | (SEQ ID | CCGGACAAGACAACTTTTTCCACCCCA (SEQ ID |
| | | | | NO: 188) | NO: 189) |
| crRNA | Paramyxoviridae--- | 1 | 1 | Cgacuccggac | AAAATCGTGAGGGGGAAGCTGGTGGACTCCGGGT |
| 82 | Avulavirus--- | | | ccggaguccac | CCGGAGTCGGTGGACCTGAGTCTAGTAGCTTCCCT |
| | Avian_avulavirus_6--- | | | cagcuu (SEQ | GCTGTGCCAAGATGTCGTCAGTGTTCAC (SEQ ID |
| | NA.aligned.p90.p3.1.tsv | | | ID NO: 190) | NO: 191) |
| crRNA | Paramyxoviridae--- | 1 | 1 | Uacuuccucc | CACTACTCCCGAGGACAATGATTCTATCAACCAGG |
| 83 | Henipavirus--- | | | ugguugauag | AGGAAGTAGTTGGGGACCCGTCTGATCAGGGTTT |
| | Hendra_henipavirus--- | | | aaucauug | AGAGCATCCTTTCCCTTTGGGGAAATTCCCGGAGA |
| | NA.aligned.p90.p3.1.tsv | | | (SEQ ID | AAGAAGAAACTCCTGATGTACGCAG (SEQ ID |
| | | | | NO: 192) | NO: 193) |
| crRNA | Paramyxoviridae--- | 1 | 1 | Gcaaagcucca | CTAAATTTGCCCCTGGAGGTTACCCATTATTGTGGA |
| 84 | Henipavirus--- | | | caauaauggg | GCTTTGCCATGGGTGTGGCTACTACTATTGACAGG |
| | Nipah_henipavirus--- | | | uaaccuc | TCTATGGGGGCATTGAATATCAATCGTGGTTATCTT |
| | NA.aligned.p90.p3.1.tsv | | | (SEQ ID | GAGCC (SEQ ID NO: 195) |
| | | | | NO: 194) | |
| crRNA | Paramyxoviridae--- | 1 | 1 | Ccaaaaccagg | AGGGGCATCTATCAAGCATTATGATAGCTATACCT |
| 85 | Morbillivirus--- | | | uauagcuauc | GGTTTTGGGAAGGACACTGGAGACCCTACGGCAA |
| | Canine_morbillivirus--- | | | auaaugc | ATGTCGACATTAACCCAGAGC (SEQ ID NO: 197) |
| | NA.aligned.p90.p3.1.tsv | | | (SEQ ID | |
| | | | | NO: 196) | |
| crRNA | Paramyxoviridae--- | 1 | 1 | Aucccucgaga | AAGCTGGTAATCCTGGAGAATTGACTTTTGCATCT |
| 86 | Morbillivirus--- | | | ugcaaaaguca | CGAGGGATTAATTTAGATAAGCAAGCTCAACAATA |
| | Feline_morbillivirus--- | | | auucuc (SEQ | CTTTAAACTGGCTGAGAAAAATGATCAGGGGTATT |
| | NA.aligned.p90.p3.1.tsv | | | ID NO: 198) | ATGTTAGCTTAGGATTTGAGAACCCACCA (SEQ ID |
| | | | | NO: 199) | |
| crRNA | Paramyxoviridae--- | 1 | 1 | Uuuuucccga | GACAGCTGCTGAAGGAATTTCAACTAAAGCCGATC |
| 87 | Morbillivirus--- | | | ucggcuuuag | GGGAAAAAGATGAGCTCAGCCGTCGGGTTTGTTC |
| | Measles_morbfflivirus--- | | | uugaaauu | CTGACACCGGCCCTGCATCACGCAGTGTAATCCGC |
| | NA.aligned.p90.p3.1.tsv | | | (SEQ ID | TCCATTATAAAATCCAGCCGGCTAG (SEQ ID |
| | | | | NO: 200) | NO: 201) |
| crRNA | Paramyxoviridae--- | 1 | 1 | Uucaccgcug | AGAGAAAGCAACAGCTGTGATGGGGAGCTGGGA |
| 88 | Morbillivirus--- | | | ugaucagaaac | GCACTCATGGATGACCTCCCAGTGCACAATACCGA |
| | Rinderpest_morbillivirus--- | | | augauaa | GGTACAGTGTTATCATGTTTCTGATCACAGCGGTG |
| | NA.aligned.p90.p3.1.tsv | | | (SEQ ID | AAAAGGTTGAGGGAGTCGAAGATGCTGACTCTAT |
| | | | | NO: 202) | CCTGGT (SEQ ID NO: 203) |
| crRNA | Paramyxoviridae--- | 1 | 1 | Cagaguauac | CACGTGGGCAACTTTAGAAGAAAGAAGAACGAAG |
| 89 | Morbillivirus--- | | | uucguucuuc | TATACTCTGCTGATTACTGCAAAATGAAGATTGAA |
| | Small_ruminant_morbillivirus | | | uuucuucu | AAGATGGGTTTAGTTTTTGCCCTGGGAGGA (SEQ |
| | --- | | | (SEQ ID | ID NO: 205) |
| | NA.aligned.p90.p3.1.tsv | | | NO: 204) | |
| crRNA | Paramyxoviridae--- | 1 | 1 | Cuguaauaau | GAGGACACAGAAGAGAGCACTCGATTTACAGAAA |
| 90 | Respirovirus--- | | | guaaucgcccu | GGGCGATTACATTATTACAGAATCTTGGTGTAATC |
| | Bovine_respirovirus_3--- | | | uucugua | CAATCTGCA (SEQ ID NO: 207) |
| | NA.aligned.p90.p3.1.tsv | | | (SEQ ID | |
| | | | | NO: 206) | |
| crRNA | Paramyxoviridae--- | 1 | 1 | Ucuacugucc | CTGCAGGGATAGGAGGAATTTAACAGGATAATTG |
| 91 | Respirovirus--- | | | aauuauccug | GACAGTAGAAACCAGATCAAAAGTAAGAAAAACT |
| | Human_respirovirus_1--- | | | uuaaauuc | TAGGGTGAATGACAATTCACAGATCAGCTCAACCA |
| | NA.aligned.p90.p3.1.tsv | | | (SEQ ID | GACATCATCAGCATACACGAAACCAACCTTCACAG |
| | | | | NO: 208) | TGGAT (SEQ ID NO: 209) |
| crRNA | Paramyxoviridae--- | 1 | 1 | Ccuaaacauga | TTGAAGACCTTGTCCACACGTTTGGGTATCCATCAT |
| 92 | Respirovirus--- | | | uggauacccaa | GTTTAGGAGCTATTATAATACAGATCTGGATAGTT |
| | Human_respirovirus_3--- | | | acgugu (SEQ | TTGGTCAAAGCTATCACTAGCATCTCAGGGT (SEQ |
| | NA.aligned.p90.p3.1.tsv | | | ID NO: 210) | ID NO: 211) |
| crRNA | Paramyxoviridae--- | 1 | 1 | Ugagacugug | GGGAGGAGGTGCTGTTATCCCCGGCCAGAGGAGC |
| 93 | Respirovirus--- | | | cuccucuggcc | ACAGTCTCAGTGTTCGTACTAGGCCCAAGTGTGAC |
| | Murine_respirovirus--- | | | ggggaua | TGATGATGCAGACAAGTTATTCATTGCAACCACCTT |
| | NA.aligned.p90.p3.1.tsv | | | (SEQ ID | CNTAGC (SEQ ID NO: 213) |
| | | | | NO: 212) | |
| crRNA | Paramyxoviridae--- | 1 | 1 | Ccgcagaugcu | GCAAGTTCACCTGCACATGCGGATCCTGCCCCAGC |
| 94 | Rubulavirus--- | | | ggggcaggauc | ATCTGCGGAGAATGTGAGGGAGATCATTGAGCTC |
| | Human_rubulavirus_2--- | | | cgcaug (SEQ | TTAAAGGGGCTTGATCTTCGCCTTCAGAC (SEQ ID |
| | NA.aligned.p90.p3.1.tsv | | | ID NO: 214) | NO: 215) |
| crRNA | Paramyxoviridae--- | 1 | 1 | Uaguuucuga | CCATGGGAGTTGGAAGTGTCCAGGATCCATTGATC |
| 95 | Rubulavirus--- | | | ucaauggaucc | AGAAACTATCAGTTTGGAAGGAACTTCTTAAATAC |
| | Human_rubulavirus_4--- | | | uggacac | CAGNTATTTTCAGTATGGTGTTGAGACTGCAATGA |
| | NA.aligned.p90.p3.1.tsv | | | (SEQ ID | AACACCAGG (SEQ ID NO: 217) |
| | | | | NO: 216) | |
| crRNA | Paramyxoviridae--- | 1 | 1 | Aaauagagau | AGGCCCAAGATGCTATCATTGGCTCAATCCTCAAT |
| 96 | Rubulavirus--- | | | ugaggauuga | CTCTATTTGACCGAGTTGACAACTATCTTCCACAAT |
| | Mammalian_rubulavirus_5- | | | gccaauga | CAAATTACAAACCCTGCATTGAGTCCTATTACAATT |
| | --NA.aligned.p90.p3.1.tsv | | | (SEQ ID | CAAGCTTTAAGGATCCTACTGGGGAG (SEQ ID |
| | | | | NO: 218) | NO: 219) |
| crRNA | Paramyxoviridae--- | 1 | 1 | Uugcaggagu | TATGCTCACCTATCACTGCCGCAGCAAGATTCCACT |
| 97 | Rubulavirus--- | | | ggaaucuugc | CCTGCAAATGTGGGAATTGCCCAGCAAAGTGCGAT |
| | Mumps_rubulavirus--- | | | ugcggcag | CAGTGCGAACGAGATT (SEQ ID NO: 221) |
| | NA.aligned.p90.p3.1.tsv | | | (SEQ ID | |
| | | | | NO: 220) | |
| crRNA | Parvoviridae--- | 1 | 1 | Cgccuggggug | GAACTCAGTGAAAGCAGCTTTTTTAACCTCATCACC |
| 98 | Erythroparvovirus--- | | | augagguuaa | CCAGGCGCCTGGAACACTGAAACCCCGCGCTCTAG |
| | Primate_erythroparvovirus_ | | | aaaagcu | TACGCCCATCCCCGGGACCAGTTCAGGAGAATCAT |
| | 1--- | | | (SEQ ID | TTGTCGGAAGCCCAGTTTCCTCCGAAGTTGTAGC |
| | NA.aligned.p90.p3.1.tsv | | | NO: 222) | (SEQ ID NO: 223) |
| crRNA | Peribunyaviridae--- | 1 | 1 | Auuugacccc | CATAAGACGCCACAACCAAGTGTCGATCTTACTTTT |
| 99 | Orthobunyavirus--- | | | ugcaaaaguaa | GCAGGGGTCAAATTTACAGTGGTTAATAACCATTT |
| | Akabane_orthobunyavirus- | | | gaucgac | TCCCCAGTACACTGCAAATCCAGTGTCAGA (SEQ |
| | --S.aligned.p90.p3.1.tsv | | | (SEQ ID | ID NO: 225) |
| | | | | NO: 224) | |
| crRNA | Peribunyaviridae--- | 1 | 1 | Cguccuuuaa | TTAAGCGTATCCACACCACTGGGCTTAGTTATGAC |
| 100 | Orthobunyavirus--- | | | uguagaagau | CACATTCGAATCTTCTACATTAAAGGACGCGAGAT |
| | Bunyamwera_orthobunyavirus | | | ucgaaugu | |
| | --- | | | (SEQ ID | TAAAACTAGTCTCGCAAAAAGAAGTGAATGGGAG |
| | S.aligned.p90.p3.1.tsv | | | NO: 226) | GTTACGCTTAACCTTGGGGG (SEQ ID NO: 227) |
| crRNA | Peribunyaviridae--- | 1 | 1 | Cuguuuccag | AAATTTGGAGAGTGGCAGGTGGAGGTTGTCAATA |
| 101 | Orthobunyavirus--- | | | gaaaaugauu | ATCATTTTCCTGGAAACAGGAACAACCCAATTGGT |
| | California_encephalitis_ | | | auugacaa | AACAACGATCTTACCATCCA (SEQ ID NO: 229) |
| | orthobunyavirus--- | | | (SEQ ID | |
| | S.aligned.p90.p3.1.tsv | | | NO: 228) | |
| crRNA | Peribunyaviridae--- | 1 | 1 | Acuuacucua | CAGTCCAGTCCTCGATGATTCATTCACACTTCATAG |
| 102 | Orthobunyavirus--- | | | ugaaguguga | AGTAAGTGGTTACCTGGCAAGGTACTTACTTGAAA |
| | Guaroa_orthobunyavirus--- | | | augaauca | GATATTTAACTGTATCAGCACCTGAGCAAG (SEQ |
| | S.aligned.p90.p3.1.tsv | | | (SEQ ID | ID NO: 231) |
| | | | | NO: 230) | |
| crRNA | Peribunyaviridae--- | 1 | 1 | Ugccuccggau | CGATGTACCACAACGGACTACATCTACATTTGATCC |
| 103 | Orthobunyavirus--- | | | caaauguaga | GGAGGCAGCATATGTGGCATTTGAAGCTAGATAC |
| | Oropouche_orthobunyavirus | | | uguaguc | GGACAAGTGCTCA (SEQ ID NO: 233) |
| | ---S.aligned.p90.p3.1.tsv | | | (SEQ ID | |
| | | | | NO: 232) | |
| crRNA | Peribunyaviridae--- | 1 | 1 | Cucucuaccaa | TGCTGATCTTCTCATGGCTAGACATGACTACTTTGG |
| 104 | Orthobunyavirus--- | | | aguagucaug | TAGAGAGGTATGTTATTACCTGGATATCGAATTCC |
| | Sathuperi_orthobunyavirus | | | ucuagcc | GGCAGGATGTTCCAGCTTACGACATACTTCTTGAA |
| | ---L.aligned.p90.p3.1.tsv | | | (SEQ ID | TTTCTGCCAGCTGGCACTGCTTTCAACATTCGC |
| | | | | NO: 234) | (SEQ ID NO: 235) |
| crRNA | Peribunyaviridae--- | 1 | 1 | Auaaaugccac | ATCTCGCTACGTTTAACCCGGAGGTCGGGTATGTG |
| 105 | Orthobunyavirus--- | | | auacccgaccu | GCATTTATTGCTAAACATGGGGCCCAACTCAATTTC |
| | Shuni_orthobunyavirus--- | | | ccgggu (SEQ | GATACCGTTAGAGTCTTCTTCCTCAATCAGAAGAA |
| | S.aligned.p90.p3.1.tsv | | | ID NO: 236) | GGCCAAGATGGTACTCAGTAAGACGGC (SEQ ID |
| | | | | | NO: 237) |
| crRNA | Phenuiviridae--- | 6 | 5 | Gauaauucag | GGCTCTTGGTGTCAAATGGTTTCACTAATTGGTGC |
| 106 | Phlebovirus--- | | | caccuauuaau | AGAATTATCAGCATCAGTTAAACAGCATGTGGGGA |
| | Candiru_phlebovirus--- | | | gagacca | AAGGCC (SEQ ID NO: 239) |
| | L.aligned.p90.p3.6.tsv | | | (SEQ ID | |
| | | | | NO: 238) | |
| crRNA | Phenuiviridae--- | 1 | 1 | Ucagaagcaa | TGGAGACAATAGCCAGGTCCATAGGGAAGTTCTTT |
| 107 | Phlebovirus--- | | | agaacuucccu | GCTTCTGATACCCTCTGTAACCCCCCCAATAAAGTG |
| | Rift_Valley_fever_phlebovirus | | | auggacc | AAAATTCCTGAGACACATGGCATCAGGGCTCGGA |
| | ---L.aligned.p90.p3.1.tsv | | | (SEQ ID | AGCAATGTAAGGGGCCTGTGTGGACTTGTGCAAC |
| | | | | NO: 240) | ATC (SEQ ID NO: 241) |
| crRNA | Phenuiviridae--- | 1 | 1 | Ggcaucgacag | CAAATCTACGACAGGCCAGGGCTGCCAGACCTAG |
| 108 | Phlebovirus--- | | | ucacaucuagg | ATGTGACTGTCGATGCCACAGGTGTGACAGTGGA |
| | SFTS_phlebovirus--- | | | ucuggc (SEQ | CATAGGGGCTGTGCCAGACTCAGCATCACAACTGG |
| | L.aligned.p90.p3.1.tsv | | | ID NO: 242) | GTTCATCAATCAATGCTGGGTTGATCACA (SEQ ID |
| | | | | | NO: 243) |
| crRNA | Phenuiviridae--- | 2 | 2 | Ucacaugggu | TTGAGTCATGCAAAGGTGTTACTACATCATCAGCC |
| 109 | Phlebovirus--- | | | accugcugcag | TCTAAGTGCTCTGGGGATGAATATTTCTGCAGCAG |
| | Sandfly_fever_Naples_ | | | aaauauu | GTACCCATGTGAAACAGCAAATGTTGAAGCCCACT |
| | phlebovirus--- | | | (SEQ ID | GCATTCTANGAAGGCATAGTGCA (SEQ ID |
| | M.aligned.p90.p3.2.tsv | | | NO: 244) | NO: 245) |
| crRNA | Phenuiviridae--- | 3 | 3 | Agagaggucac | ATGGGGCCCAGCATGCTACATCAGTTCTGTNAAGC |
| 110 | Phlebovirus--- | | | uugccaugccu | CTATGGTGTACACCTTCCAAGGCATGGCAAGTGAC |
| | Sandfly_fever_Sicilian_virus | | | uggaag (SEQ | CTCTCTAGGTTTGANCTGACTAGTTTCTCTANGAG |
| | ---S.aligned.p90.p3.3.tsv | | | ID NO: 246) | AGGACTGCCAAATGTTNTGAAAGCTCTNAGCTGG |
| | | | | | CCAC (SEQ ID NO: 247) |
| crRNA | Phenuiviridae--- | 3 | 3 | ugggccagcuc | GATTTGATGCTGCTGTGGTCCTGAGGAGGATTTTN |
| 111 | Phlebovirus--- | | | Naaaauccuc | GAGCTGGCCCANAAAGCTGGNCTGGACANGGACC |
| | Uukuniemi_phlebovirus--- | | | cucagga | AGATGATGAGGGACA (SEQ ID NO: 249) |
| | S.aligned.p90.p3.3.tsv | | | (SEQ ID | |
| | | | | NO: 248) | |
| crRNA | Picornaviridae--- | 1 | 1 | Uguuaccucg | TGGTGACAGGCTAAGGATGCCCTTCAGGTACCCCG |
| 112 | Aphthovirus---Foot-and- | | | ggguaccugaa | AGGTAACACGCGACACTCGGGATCTGAGAAGGGG |
| | mouth_disease_virus--- | | | gggcauc | ACTGGGGCTTCTTTAAAAGCGCCCAGTTTAAAAAG |
| | NA.aligned.p90.p3.1.tsv | | | (SEQ ID | CTTCTATGCCTGAATAGGTGACCGGAG (SEQ ID |
| | | | | NO: 250) | NO: 251) |
| crRNA | Picornaviridae--- | 1 | 1 | Caauggggua | TATTCAACAAGGGGCTGAAGGATGCCCAGAAGGT |
| 113 | Cardiovirus--- | | | ccuucugggca | ACCCCATTGTATGGGATCTGATCTGGGGCCTCGGT |
| | Cardiovirus_A--- | | | uccuuca | GCACATGCTTTACATGTGTTTAGTCGAGGTTAAAA |
| | NA.aligned.p90.p3.1.tsv | | | (SEQ ID | AACGTCTAGGCCCCCCNAACCACGGGGACGTGGT |
| | | | | NO: 252) | TTTCCTTTG (SEQ ID NO: 253) |
| crRNA | Picornaviridae--- | 1 | 1 | Cccagcagggc | TATCATGCCTCCCCGATTATGTGATGTTTTCTGCCC |
| 114 | Cardiovirus--- | | | agaaaacauca | TGCTGGGCGGAGCATTCTCGGGTTGAGAAACCTTG |
| | Cardiovirus_B--- | | | cauaau (SEQ | AATCTTTTCCTTTGGAACCTTGGTTCCCCCGGTCTA |
| | NA.aligned.p90.p3.1.tsv | | | ID NO: 254) | AGCCGCTTGGAATATGA (SEQ ID NO: 255) |
| crRNA | Picornaviridae--- | 6 | 5 | Uguguucucc | CATTCATGTCACCTGCGAGTGCTTATCAATGGTTTT |
| 115 | Enterovirus--- | | | gaauguggga | ATGACGGATATCCCACATTCGGAGAACACAAACAG |
| | Enterovirus_A--- | | | uauccguc | GAGAAAGATCTTGAATATGGGGCATGTCCTAATAA |
| | NA.aligned.p90.p3.6.tsv | | | (SEQ ID | CATGATGGGCACTTT (SEQ ID NO: 257) |
| | | | | NO: 256) | |
| crRNA | Picornaviridae--- | 1 | 3 | Gcugcagagu | ATGCGGCTAATCCTAACTGCGGAGCAGATACCCAC |
| 116 | Enterovirus--- | | | ugcccguuacg | AAACCAGTGGGCAGTCTGTCGTAACGGGCAACTCT |
| | Enterovirus_B--- | | | acagacu | GCAGCGGAACCGACTACTTTGGGTG (SEQ ID |
| | NA.aligned.p90.p3.1.tsv | | | (SEQ ID | NO: 259) |
| | | | | NO: 258) | |
| crRNA | Picornaviridae--- | 1 | 2 | Caauccaauuc | CGACTACTTTGGGTGTCCGTGTTTCCTTTTATTTTAT |
| 117 | Enterovirus--- | | | gcuuuaugau | AATGGCTGCTTATGGTGACAATCATAGATTGTTAT |
| | Enterovirus_C--- | | | aacaauc | CATAAAGCGAATTGGATTGGCCA (SEQ ID |
| | NA.aligned.p90.p3.1.tsv | | | (SEQ ID | NO: 261) |
| | | | | NO: 260) | |
| crRNA | Picornaviridae--- | 1 | 1 | Aauugucccg | CTCAAGGTGTCCCAACATACCTTTTACCAGGCTCG |
| 118 | Enterovirus--- | | | agccugguaaa | GGACAATTCCTAACAACTGATGATCATAGCTCTGC |
| | Enterovirus_D--- | | | agguaug | ACCAGCTCTCCCGTGTTTCAACCCAACTCC (SEQ ID |
| | NA.aligned.p90.p3.1.tsv | | | (SEQ ID | NO: 263) |
| | | | | NO: 262) | |
| crRNA | Picornaviridae--- | 1 | 1 | Gcaacacugga | GCTAATCCCAACCTCCGAGCGTGTGCGCACAATCC |
| 119 | Enterovirus--- | | | uugugcgcaca | AGTGTTGCTACGTCGTAACGCGTAAGTTGGAGGC |
| | Enterovirus_E--- | | | cgcucg (SEQ | GGAACAGACTACTTT (SEQ ID NO: 265) |
| | NA.aligned.p90.p3.1.tsv | | | ID NO: 264) | |
| crRNA | Picornaviridae--- | 1 | 1 | Acacccaaagu | GCCCCTGAATGTGGCTAACCTTAACCCTGCAGCCA |
| 120 | Enterovirus---Rhinovirus_A- | | | aguugguccca | GTGCACACAATCCAGTGTGTATCTGGTCGTAATGA |
| | --NA.aligned.p90.p3.1.tsv | | | ucccgc (SEQ | GCAATTGCGGGATGGGACCAACTACTTTGGGTGTC |
| | | | | ID NO: 266) | CG (SEQ ID NO: 267) |
| crRNA | Picornaviridae--- | 1 | 5 | Uggauuguga | CCCTGAATGCGGCTAACCTTAACCCCGGAGCCTTG |
| 121 | Enterovirus---Rhinovirus_B- | | | ugcaaggcucc | CGGCACAATCCAGTGTTGTTAAGGTCGTAATGAGC |
| | --NA.aligned.p90.p3.1.tsv | | | gggguua | AATTCTGGGATGGGACCGACTACTTTG (SEQ ID |
| | | | | (SEQ ID | NO: 269) |
| | | | | NO: 268) | |
| crRNA | Picornaviridae--- | 1 | 1 | Acauacaugc | GCCCCTGAATGCGGCTAATCCTAACCCCGCAGCTA |
| 122 | Enterovirus---Rhinovirus_C- | | | uggcuugcau | TTGCATGCAAGCCAGCATGTATGTAGTCGTAATGA |
| | --NA.aligned.p90.p3.1.tsv | | | gcaauagc | GCAATTGTGGGATGGAACCGACTACTTTGGGTG |
| | | | | (SEQ ID | (SEQ ID NO: 271) |
| | | | | NO: 270) | |
| crRNA | Picornaviridae--- | 1 | 1 | Agccuaccccu | GAGTCTAAATTGGGGACGCAGATGTTTGGGACGT |
| 123 | Hepatovirus--- | | | uguggaagau | CACCTTGCAGTGTTAACTTGGCTTTCATGAACCTCT |
| | Hepatovirus_A--- | | | caaagag | TTGATCTTCCACAAGGGGTAGGCTACGGGTGAAAC |
| | NA.aligned.p90.p3.1.tsv | | | (SEQ ID | CTCTTAGGC (SEQ ID NO: 273) |
| | | | | NO: 272) | |
| crRNA | Picornaviridae---Kobuvirus- | 1 | 1 | Gcaaccacauc | CACGATCTATGAAGTCACCTTCCTCAAGCGCTGGTT |
| 124 | --Aichivirus_A--- | | | acugauuguu | CGTTCCGGACGACGTTAGGCCCATCTACATCCACC |
| | NA.aligned.p90.p3.1.tsv | | | cguacgu | CTGTGATGGACCCTGACACGTACGAACAATCAGTG |
| | | | | (SEQ ID | ATGTGGTTGCGTGATGGAGATTT (SEQ ID NO: 275) |
| | | | | NO: 274) | |
| crRNA | Picornaviridae--- | 1 | 1 | Ccuuacaacua | GGCCAAAAGCCAAGGTTTAACAGACCCTTTAGGAT |
| 125 | Parechovirus--- | | | guguuugcau | TGGTTCAAACCTGAAATGTTNTGGAAGATATTTAG |
| | Parechovirus_A--- | | | uacuacc | TACCTGCTGATTTGGTAGTAGTGCAAACACTAGTT |
| | NA.aligned.p90.p3.1.tsv | | | (SEQ ID | GTAAGGCCCACGAAGGATGCCCAGAAGGTA (SEQ |
| | | | | NO: 276) | ID NO: 277) |
| crRNA | Pneumoviridae | 1 | 1 | Auuccacaauc | AGAGGTGGCTCCAGAATACAGGCATGACTCTCCTG |
| 126 | Respiratory_syncytial_virus | | | aggagagucau | ATTGTGGAATGATAATATTATGTATAGCAGCATTA |
| | ---NA.aligned.p90.p3.1.tsv | | | gccugu (SEQ | GTAATAACCAAATTAGCAGCAGGGGATAGA (SEQ |
| | | | | ID NO: 278) | ID NO: 279) |
| crRNA | Pneumoviridae--- | 1 | 2 | Gcuugaguua | AAGCTGCAATTAGTGGGGAAGCAGATCAAGCTAT |
| 127 | Metapneumovirus--- | | | uagcuugauc | AACTCAAGCTAGGATTGCTCCATACGCTGGNTTGA |
| | Avian_metapneumovirus--- | | | ugccuccc | TCATGATAATGACAATGAACAACCCTAA (SEQ ID |
| | NA.aligned.p90.p3.1.tsv | | | (SEQ ID | NO: 281) |
| | | | | NO: 280) | |
| crRNA | Pneumoviridae--- | 1 | 1 | Ucauaaucau | AAAAAGAGGCTGCAGAACACTTCCTAAATGTGAGT |
| 128 | Metapneumovirus--- | | | uuugacuguc | GACGACAGTCAAAATGATTATGAGTAATTAAAAAA |
| | Human_metapneumovirus- | | | gucacuca | GTGGGACAAGTCAAAATGTCATTCCCTGAAGGAA |
| | --NA.aligned.p90.p3.1.tsv | | | (SEQ ID | AAGATATTCTTTTCATGGGTAATGAAGCAGCAA |
| | | | | NO: 282) | (SEQ ID NO: 283) |
| crRNA | Pneumoviridae--- | 1 | 1 | Gccuucguga | TGGGGCAAATATGGAAACATACGTGAACAAACTTC |
| 129 | Orthopneumovirus--- | | | agcuuguucac | ACGAAGGCTCCACATACACAGCTGCTGTTCAATAC |
| | Human_orthopneumovirus | | | guauguu | AATGTCCTAGAAAAAGACGATGATCCTGCATCACT |
| | ---NA.aligned.p90.p3.1.tsv | | | (SEQ ID | TACAATATGGGTGCC (SEQ ID NO: 285) |
| | | | | NO: 284) | |
| crRNA | Polyomaviridae--- | 1 | 1 | Uguaagcaag | TTATTTGGTGCTTGCCTGATACAACCTTTAAGCCTT |
| 130 | Alphapolyomavirus--- | | | gcuuaaaggu | GCTTACAAGAAGAAATTAAAAACTGGAAGCAAATT |
| | Human_polyomayirus_5--- | | | uguaucag | TTACAGAGTGAAATATCATATGGTAAATTTTGTCA |
| | NA.aligned.p90.p3.1.tsv | | | (SEQ ID | AATGATAGAAAATGTAGAAGCTGGTCAGGAC |
| | | | | NO: 286) | (SEQ ID NO: 287) |
| crRNA | Polyomaviridae--- | 1 | 1 | Uuggucacau | TCACAGGAGGGGAAAATGTTCCCCCAGTACTTCAT |
| 131 | Betapolyomavirus | | | gaaguacuggg | GTGACCAACACAGCTACCACAGTGTTGCTAGATGA |
| | Human_polyomavirus_1--- | | | ggaacau | ACAGGGTGTGGGGCCTCTTTGTAAAG (SEQ ID |
| | NA.aligned.p90.p3.1.tsv | | | (SEQ ID | NO: 289) |
| | | | | NO: 288) | |
| crRNA | Polyomaviridae--- | 1 | 1 | Ugccauacau | AACAGAAGGACCCCTAGAGTTGATGGGCAGCCTA |
| 132 | Betapolyomavirus | | | aggcugcccau | TGTATGGCATGGATGCTCAAGTAGAGGAGGTTAG |
| | Human_polyomavirus_2--- | | | caacucu | AGTTTTTGAGGGGACAGAGGAACTTCCAGGGGAC |
| | NA.aligned.p90.p3.1.tsv | | | (SEQ ID | CCAGACATGATGAG (SEQ ID NO: 291) |
| | | | | NO: 290) | |
| crRNA | Polyomaviridae--- | 1 | 1 | Uauagguagu | GGTGTAACACCCACAGACAAGTATAAAGGCCCAAC |
| 133 | Betapolyomavirus--- | | | ugggccuuua | TACCTATACAATTAATCCACCAGGAGACCCTAGAA |
| | Human_polyomavirus_3--- | | | uacuuguc | CACTGC (SEQ ID NO: 293) |
| | NA.aligned.p90.p3.1.tsv | | | (SEQ ID | |
| | | | | NO: 292) | |
| crRNA | Polyomaviridae--- | 1 | 1 | Agugaaacuu | CAATTAGCAGCCACAAGGTGGAGCAAAAGTATTA |
| 134 | Betapolyomavirus--- | | | aauacuuuug | AGTTTCACTGTTATGTGCAGGAATGTGCAGCTGTG |
| | Human_polyomavirus_4--- | | | cuccaccu | ACCTTTTA (SEQ ID NO: 295) |
| | NA.aligned.p90.p3.1.tsv | | | (SEQ ID | |
| | | | | NO: 294) | |
| crRNA | Polyomaviridae--- | 1 | 1 | Caaaaagcuu | ATTGGGGTCCAACACTTTTTAATGCCATTTCTCAAG |
| 135 | Betapolyomavirus--- | | | gagaaauggca | CTTTTTGGCGTGTAATACAAAATGACATTCCTAGGC |
| | Macaca_mulatta_ | | | uuaaaaa | TCACC (SEQ ID NO: 297) |
| | polyomavirus_1--- | | | (SEQ ID | |
| | NA.aligned.p90.p3.1.tsv | | | NO: 296) | |
| crRNA | Poxviridae---Orthopoxvirus- | 1 | 2 | Gcuugaguua | GCTACGGGCATTGTCATCTTTAAAACTCTCCACTTT |
| 136 | --Cowpox_virus--- | | | uagcuugauc | CCATCTTCTGGAGATCTTCTTTCAATGGTAGGATTA |
| | NA.aligned.p90.p3.1.tsv | | | ugccuccc | TAATATCTGTTGTTATAATCGTAATATCCACAATCA |
| | | | | (SEQ ID | GGATCTGTAAAGCGAGC (SEQ ID NO: 299) |
| | | | | NO: 298) | |
| crRNA | Poxviridae---Orthopoxvirus- | 1 | 1 | Ucacgacgagg | CCACCGCAATAGATCCTGTTAGATACATAGATCCTC |
| 137 | --Monkeypox_virus--- | | | aucuauguau | GTCGTGATATCGCATTTTCTAACGTGATGGATATAT |
| | NA.aligned.p90.p3.1.tsv | | | cuaacag | TAAAGTCGAATAAAGTTGAACAATAATTAATTCTTT |
| | | | | (SEQ ID | ATTGTTATCATGAACGGCGGACATATT (SEQ ID |
| | | | | NO: 300) | NO: 301) |
| crRNA | Poxviridae---Orthopoxvirus- | 1 | 1 | Aauccaucuca | GACACGCTGGACAATCTAGCATTCACTGTGTTTCC |
| 138 | --Vaccinia_virus--- | | | gaauccgcuga | ATCAGCGGATTCTGAGATGGATTTAATCTGAGGAC |
| | NA.alignecip90.p3.1.tsv | | | uggaaa (SEQ | ATTTGGTGAATCCAAAGTTCATTCTCAGACCTCCAC |
| | | | | ID NO: 302) | C (SEQ ID NO: 303) |
| crRNA | Poxviridae---Orthopoxvirus- | 1 | 1 | Aagaaucaau | TGGACCCCAACATCTTTGACCGATTAAGTTTTGATT |
| 139 | --Variola_virus--- | | | caaaacuuaau | GATTCTTCCATGTAAGGCGTATCTAGTCAGATCGT |
| | NA.aligned.p90.p3.1.tsv | | | cggucaa | ATAATCTAGCCAACAATCCATCGTCGGTGTTTAGG |
| | | | | (SEQ ID | TC (SEQ ID NO: 305) |
| | | | | NO: 304) | |
| crRNA | Poxviridae---Parapoxvirus-- | 1 | 1 | Auggauccacc | CGGCAACCCCGATTATGTAGGCCGTGATTTCGGGT |
| 140 | -Orf_virus--- | | | cgaaaucacgg | GGATCCATTTAGTTATTAAAATTAATCATATACAAC |
| | NA.aligned.p90.p3.1.tsv | | | ccuaca (SEQ | TCTTTTATGGCGGCTATGGATTCGGCTATCCAGTCC |
| | | | | ID NO: 306) | TTGAC (SEQ ID NO: 307) |
| crRNA | Reoviridae---Orbivirus--- | 2 | 1 | Gcgugucgua | TAATCGGCGACCTNGAAGCGACNGGATCGCGNGT |
| 141 | Greatisland_virus--- | | | guuugaguag | GATGGATGCGGCAGANACCTTCCGCAANACCGGT |
| | 1.aligned.p90.p3.2.tsv | | | uccagggc | GACGTTGGGATATGGACATTAGCCCTGGACTACTC |
| | | | | (SEQ ID | NAANTACGACACGCACAT (SEQ ID NO: 309) |
| | | | | NO: 308) | |
| crRNA | Reoviridae---Orthoreovirus- | 2 | 1 | Cgacagccaaa | GGACTGCCGAATACCTAAAGCTGTACTTCATATTT |
| 142 | -- | | | uaugaaguac | GGCTGTCGAATTCCAAATCTCAGTCGTCATCCAATC |
| | Mammalian_orthoreovirus- | | | agcuuua | GTGGG (SEQ ID NO: 311) |
| | --L1.aligned.p90.p3.2.tsv | | | (SEQ ID | |
| | | | | NO: 310) | |
| crRNA | Reoviridae---Rotavirus--- | 1 | 1 | Aucuaaucga | TTGGACCATCTGATTCTGCTTCAAACGATCCACTCA |
| 143 | Rotavirus_A--- | | | aaagcugguga | CCAGCTTTTCGATTAGATCGAATGCAGTTAAGACA |
| | 11.aligned.p90.p3.1.tsv | | | guggauc | AATGCAGACGCTGGCGTGTCTATGGATT (SEQ ID |
| | | | | (SEQ ID | NO: 313) |
| | | | | NO: 312) | |
| crRNA | Reoviridae---Rotavirus--- | 1 | 1 | Uagagcagcaa | ATATCGTGTCCTTGAGCACAGCTCAAAAGAAATTG |
| 144 | Rotavirus_B--- | | | uuucuuuuga | CTGCTCTACGGATTCACCCAACCTGGTGTACAGGG |
| | 4.aligned.p90.p3.1.tsv | | | gcugugc | TTTGACTG (SEQ ID NO: 315) |
| | | | | (SEQ ID | |
| | | | | NO: 314) | |
| crRNA | Reoviridae---Rotavirus--- | 3 | 2 | Uuaaaucagg | CACATGCTGATTACGTTTCAGCTAGAAGATTTATAC |
| 145 | Rotavirus_C--- | | | uauaaaucuu | CTGATTTAACTGAACTGGTTGATGCTGAAAAACAA |
| | 2.aligned.p90.p3.3.tsv | | | cuagcuga | ATAAAAGAAATGGCTGCACA (SEQ ID NO: 317) |
| | | | | (SEQ ID | |
| | | | | NO: 316) | |
| crRNA | Reoviridae---Rotavirus--- | 1 | 1 | Caagugcgug | ATCTACTTGCACCAGGTGGAGCAACGAATAACACT |
| 146 | Rotavirus_H--- | | | auauccuccac | GGTGGAGGATATCACGCACTTGTTGGAAGAGCTA |
| | 6.aligned.p90.p3.1.tsv | | | caguguu | CTGGAAAGATGGCTGTCGTAACTGCAGTTCAAGG |
| | | | | (SEQ ID | AAGACCCGGAGGAATCAATTTTGCACTTGACATGA |
| | | | | NO: 318) | AAGTACC (SEQ ID NO: 319) |
| crRNA | Reoviridae--- | 1 | 1 | Aaaucuuuug | CTTGATTTCCAGCACCAGTGCACTGATAGTAGTAA |
| 147 | Seadornavirus--- | | | uauugcucgu | GAAACGAGCAATACAAAAGATTTGTGTCTTAATTA |
| | Banna_virus--- | | | uucuuacu | GTAATGATCTTAGAGAGAATGGACTATTAGAAGA |
| | 12.aligned.p90.p3.1.tsv | | | (SEQ ID | GGCCAAAACATTCAAGCCAGAGTA (SEQ ID |
| | | | | NO: 320) | NO: 321) |
| crRNA | Retroviridae--- | 1 | 1 | Guuaaaacaa | TGCTAATACGCCTCCCTTTCCGGACAACGCCTATTG |
| 148 | Deltaretrovirus--- | | | uaggcguugu | TTTTAACATCTTGCCTAGTTGATACCAAAAACAACT |
| | Primate_T- | | | ccggaaag | GGGCCATCATAGGTCGTGATGCCTT (SEQ ID |
| | lymphotropic_virus_1--- | | | (SEQ ID | NO: 323) |
| | NA.aligned.p90.p3.1.tsv | | | NO: 322) | |
| crRNA | Retroviridae--- | 1 | 1 | Ugaaggcgaa | ATAGACCTTACTGACGCCTTTTTCCAAATCCCCCTC |
| 149 | Deltaretrovirus--- | | | guauggcugg | CCCAAGCAGTTCCAGCCATACTTCGCCTTCACCATT |
| | Primate_T- | | | aacugcuu | CCCCAGCCATGTAATTATGGCCCCGG (SEQ ID |
| | lymphotropic_virus_2--- | | | (SEQ ID | NO: 325) |
| | NA.aligned.p90.p3.1.tsv | | | NO: 324) | |
| crRNA | Retroviridae---Lentivirus--- | 1 | 1 | Uuucuguuaa | AATGGCCATTGACAGAAGAAAAAATAAAAGCATT |
| 150 | Human_immunodeficiency_ | | | ugcuuuuauu | AACAGAAATTTGTACAGAAATGGAAAAGGAAGGA |
| | virus_1--- | | | uuuucuuc | AAAATTTCAAAAATTGGGCCTGAAAATCCA (SEQ |
| | NA.aligned.p90.p3.1.tsv | | | (SEQ ID | ID NO: 327) |
| | | | | NO: 326) | |
| crRNA | Retroviridae---Lentivirus--- | 1 | 1 | Gucuagcagg | CGGAGAGGCTGGCAGATTGAGCCCTGGGAGGTTC |
| 151 | Human_immunodeficiency_ | | | gaacacccagg | TCTCCAGCACTAGCAGGTAGAGCCTGGGTGTTCCC |
| | virus_2--- | | | cucuacc | TGCTAGACTCTCA (SEQ ID NO: 329) |
| | NA.aligned.p90.p3.1.tsv | | | (SEQ ID | |
| | | | | NO: 328) | |
| crRNA | Retroviridae---Lentivirus--- | degen | 1 | Gcaacuauga | TGGCAAATGGATTGTACCCATCTAGAGGGAAAAAT |
| 152 | Simian_immunodeficiency_ | | | uuauuuuucc | AATCATAGTTGCAGTACATGTAGCTAGTGGATTCA |
| | virus---NA | | | cucuagau | TAGAAGCAGAAGTAATTCCACAAGAAACAGGAAG |
| | | | | (SEQ ID | ACAGACAGCACTATTTCTGTTAAAATTGGCAGGCA |
| | | | | NO: 330) | GATGGCCTATTACACATCTACACACAGATAATGGT |
| | | | | | GCTAACTTTACTTCGCAAGAAGTAAAGATGGTTGC |
| | | | | | ATGGTGGGCAGGGATAGAGCACACCTTTGGGGTA |
| | | | | | CCATACAATCCACAGAGTCA (SEQ ID NO: 331) |
| crRNA | Rhabdoviridae---Lyssavirus- | 1 | 1 | Auccaucaucc | CCAGGATTAGACTGGGCTGCCAGCAATGATGAGG |
| 153 | -- | | | ucaucauugc | ATGATGGATCTATTGAGGCAGAGATTGCCCATCAG |
| | European_bat_1_lyssavirus | | | uggcagc | ATAGCC (SEQ ID NO: 333) |
| | ---NA.aligned.p90.p3.1.tsv | | | (SEQ ID | |
| | | | | | NO: 332) |
| crRNA | Rhabdoviridae---Lyssavirus- | 1 | 1 | Cagggguucu | TCAGACGATGAGGAGCTTTACTCCGGAGGGACAA |
| 154 | -- | | | ugucccuccgg | GAACCCCTGAAGCTGTGTACACCAGGATCATGGTC |
| | European_bat_2_lyssavirus | | | aguaaag | AATGGGGGAAAG (SEQ ID NO: 335) |
| | ---NA.aligned.p90.p3.1.tsv | | | (SEQ ID | |
| | | | | NO: 334) | |
| crRNA | Rhabdoviridae---Lyssavirus- | 1 | 1 | Gauugacaaa | AACACCCCTCCTTTTGAACCATCCCAAACATGAGCA |
| 155 | --Rabies_lyssavirus--- | | | gaucuugcuca | AGATCTTTGTCAATCCGAGTGCTATCAGAGCCGGT |
| | NA.aligned.p90.p3.1.tsv | | | uguuugg | CTGGCTGATCTTGAGATGGCTGAAGAGACTGTTGA |
| | | | | (SEQ ID | TCTGATCAATAGAAACATAGAAGACAATCAGGCTC |
| | | | | NO: 336) | ATCTCCA (SEQ ID NO: 337) |
| crRNA | Rhabdoviridae--- | 1 | 1 | Cguccucuug | CAACGAGCTGAAAAGTCCAATTATGAGTTGTTCCA |
| 156 | Vesiculovirus--- | | | gaacaacucau | AGAGGACGGAGTGGAAGAGCATACTAGGCCCTCT |
| | Indiana_vesiculovirus--- | | | aauugga | TATTTTCAGGCAGCAGATGA (SEQ ID NO: 339) |
| | NA.aligned.p90.p3.1.tsv | | | (SEQ ID | |
| | | | | NO: 338) | |
| crRNA | Rhabdoviridae--- | 1 | 2 | Gagccauuuu | TATTTGGCCTAGAGGGAACTTTTAACAGATATCAA |
| 157 | Vesiculovirus--- | | | gauaucuguu | AATGGCTCCTACAGTTAAGAGAATCATTAACGACT |
| | New_Jersey_vesiculovirus-- | | | aaaaguuc | CCATTATTCAGCCTAAGTTACCGGCCAATGAGGAT |
| | -NA.aligned.p90.p3.1.tsv | | | (SEQ ID | CCGGTTGAATACCCGGCTGATTACTTCAA (SEQ ID |
| | | | | NO: 340) | NO: 341) |
| crRNA | Smacoviridae | 2 | 2 | Ugaguaucca | CCTGAACCGGTCTTCTGACAACAAGTCGTATTTTG |
| 158 | Human_smacovirus_1--- | | | aaguacgacuu | GATACTCATTTGTAAAAACAAACACTCTTGGACTGT |
| | NA.aligned.p90.p3.2.tsv | | | guuguca | CTATCCACATTTCTTCCCATGTGTACCTGTCGTCCCA |
| | | | | (SEQ ID | CATGTACCCATT (SEQ ID NO: 343) |
| | | | | NO: 342) | |
| crRNA | Togaviridae---Alphavirus--- | 1 | 1 | Uaccccguggu | GAATAACGATGAGCCCAGGCCTTTATGGAAAAACC |
| 159 | Chikungunya_virus--- | | | uuuuccauaa | ACGGGGTATGCGGTAACCCACCACGCAGACGGAT |
| | NA.aligned.p90.p3.1.tsv | | | aggccug | TCTTGATGTGCAAGACTACCGA (SEQ ID NO: 345) |
| | | | | (SEQ ID | |
| | | | | NO: 344) | |
| crRNA | Togaviridae---Alphavirus--- | 1 | 1 | Caaugcgaugc | AGCAGTGGACCATTTGAACAAAGGCGGTACGTGC |
| 160 | Eastern_equine_encephalitis | | | acguaccgccu | ATCGCATTGGGCTATGGGACTGCGGACAGAGCCA |
| | _virus--- | | | uuguuc (SEQ | CCGAGAACATTA (SEQ ID NO: 347) |
| | NA.aligned.p90.p3.1.tsv | | | ID NO: 346) | |
| crRNA | Togaviridae---Alphavirus--- | 1 | 1 | Cuuacacauca | TTACGCAGTTACCCATCACGCAGAGGGTTTCCTGA |
| 161 | Getah_virus--- | | | ggaaacccucu | TGTGTAAGATCACTGATACAGTCAGAGGAGAAAG |
| | NA.aligned.p90.p3.1.tsv | | | gcguga (SEQ | AGTCTCTTTCCCGGTCTGTAC (SEQ ID NO: 349) |
| | | | | ID NO: 348) | |
| crRNA | Togaviridae---Alphavirus--- | 1 | 1 | Gugagugcaa | ACCTGGACAGCGGATTATTTTCAGCACCCGCTGTT |
| 162 | Highlands_J_virus--- | | | cagcgggugcu | GCACTCACCTATAAGGATCATCACTGGGATAATTC |
| | NA.aligned.p90.p3.1.tsv | | | gaaaaua | GCC (SEQ ID NO: 351) |
| | | | | (SEQ ID | |
| | | | | NO: 350) | |
| crRNA | Togaviridae---Alphavirus--- | 1 | 1 | Aagaagucgg | CAGAGGTGGCAGTCTATCAGGATGTCTATGCAGTT |
| 163 | Mayaro_virus--- | | | ugcauggacug | CATGCACCGACTTCTTTGTACTTCCAGGCAATGAAA |
| | NA.aligned.p90.p3.1.tsv | | | cauagac | GGAGTACGC (SEQ ID NO: 353) |
| | | | | (SEQ ID | |
| | | | | NO: 352) | |
| crRNA | Togaviridae---Alphavirus--- | 1 | 1 | Gccacucucuc | TTCCGTGTCTGTGTAGGTACGCTATGACTGCTGAG |
| 164 | Ross_River_virus--- | | | agcagucauag | AGAGTGGCAAGACTTCGGATGAACAACACTAAGG |
| | NA.aligned.p90.p3.1.tsv | | | cguacc (SEQ | CCATAATTGTGTGCTCCTCCTTCCCTTTACCGAAGT |
| | | | | ID NO: 354) | ACAGGATTGAAGGCGTC (SEQ ID NO: 355) |
| crRNA | Togaviridae---Alphavirus--- | 1 | 1 | Ugaugguaca | AGGACGTGTATGCTGTACATGCACCAACATCGCTG |
| 165 | Semliki_Forest_virus--- | | | gcgauguugg | TACCATCAGGCGATGAAAGGTGTCAGAACGGCGT |
| | NA.aligned.p90.p3.1.tsv | | | ugcaugua | ATTGGATTG (SEQ ID NO: 357) |
| | | | | (SEQ ID | |
| | | | | NO: 356) | |
| crRNA | Togaviridae---Alphavirus--- | 1 | 1 | Uccgucgaaaa | AATACTGACTAACCGGGGTAGGTGGGTACATATTT |
| 166 | Sindbis_virus--- | | | uauguacccac | TCGACGGACACAGGCCCTGGGCACTTGCAAAAGA |
| | NA.aligned.p90.p3.1.tsv | | | cuaccc (SEQ | AGTCCGTTCTGCA (SEQ ID NO: 359) |
| | | | | ID NO: 358) | |
| crRNA | Togaviridae---Alphavirus--- | 1 | 1 | Cuggcguuag | TTTGAGGTAGAAGCCAAGCAGGTCACTGATAATG |
| 167 | Venezuelan_equine_encep | | | cauggucguu | ACCATGCTAACGCCAGAGCGTTTTCGCATCTGGCT |
| | halitis_virus--- | | | auccguga | TCAAAATTGATCGAAACGGAGGTGGACCCATCCG |
| | NA.aligned.p90.p3.1.tsv | | | (SEQ ID | ACACGATCCTTGACATTGGAAGTGCG (SEQ ID |
| | | | | NO: 360) | NO: 361) |
| crRNA | Togaviridae---Alphavirus--- | 1 | 1 | Cagugaacagg | GGCAAAGATCGAGTGATGCAATCATTGCATCACCT |
| 168 | Western_equine_encephalitis_ | | | ugaugcaaug | GTTCACTGCTTTCGACACTACGGATGCCGATGTCA |
| | virus--- | | | auugcau | CCATATATTGCTTGGATAAACAATGGGAGACCAGG |
| | NA.aligned.p90.p3.1.tsv | | | (SEQ ID | ATAATCGAGGCCATTCACC (SEQ ID NO: 363) |
| | | | | NO: 362) | |
| crRNA | Togaviridae---Rubivirus--- | 1 | 1 | Gccccacucga | CGCAATTTCGCGGTATACCCGCCGCCATTGGATCG |
| 169 | Rubella_virus--- | | | uccaauggcgg | AGTGGGGCCCTAAAGAAGCCCTACACGTCCTCATC |
| | NA.aligned.p90.p3.1.tsv | | | cgggua (SEQ | GAC (SEQ ID NO: 365) |
| | | | | ID NO: 364) | |

TABLE 4
HAV Round 1 Primers

| | | Primer |
|---|---|---|
| Name | Sequence | pool |
| Coronaviridae---Betacoronavirus--- | gtTAATACGACTCACTATAGGGCTTTGCTGAGTTG | 1 |
| Middle_East_respiratory_syndronne- | GAAGC (SEQ ID NO: 366) | |
| related_coronavirus---NA.u1.g1 | | |
| Coronaviridae---Betacoronavirus--- | AGAACTTGTGGTGAGGTG (SEQ ID NO: 367) | 1 |
| Middle_East_respiratory_syndronne- | | |
| related_coronavirus---NA.u1.g2 | | |
| Filoviridae---Ebolavirus---Sudan_ebolavirus--- | gtTAATACGACTCACTATAGGGAGTCAATCCCCCA | 1 |
| NA.u1.g1 | TTTGG (SEQ ID NO: 1064) | |
| Filoviridae---Ebolavirus---Sudan_ebolavirus--- | CCAGGTTAGGAGGCA (SEQ ID NO: 368) | 1 |
| NA.u1.g2 | | |
| Filoviridae---Ebolavirus---Zaire_ebolavirus--- | gtTAATACGACTCACTATAGGGGCCTAACAGATCG | 1 |
| NA.u1.g1 | ACCAA (SEQ ID NO: 369) | |
| Filoviridae---Ebolavirus---Zaire_ebolavirus--- | TCTGTCTGCCCTCTGTAT (SEQ ID NO: 370) | 1 |
| NA.u1.g2 | | |
| Flaviviridae---Flavivirus---Dengue_virus---NA.u1.g1 | gtTAATACGACTCACTATAGGGACGCCTTTCAATA | 1 |
| | TGCTG (SEQ ID NO: 371) | |
| Flaviviridae---Flavivirus---Dengue_virus---NA.u1.g2 | TGAGAATCTCTTTGTCAGCT (SEQ ID NO: 372) | 1 |
| Flaviviridae---Flavivirus---Dengue_virus---NA.u1.g3 | gtTAATACGACTCACTATAGGGCCGTCTTTCAATA | 1 |
| | TGCTGA (SEQ ID NO: 373) | |
| Flaviviridae---Flavivirus---Dengue_virus---NA.u1.g4 | TGAGAATCTCTTCGCCAA (SEQ ID NO: 374) | 1 |
| | gtTAATACGACTCACTATAGGGACCCCATGTGGAG | 1 |
| Flaviviridae---Flavivirus---Zika_virus---NA.u1.g1 | AG (SEQ ID NO: 375) | |
| Flaviviridae---Flavivirus---Zika_virus---NA.u1.g2 | TTCCTTCAGTGTGTCACC (SEQ ID NO: 376) | 1 |
| Herpesviridae---Simplexvirus--- | gtTAATACGACTCACTATAGGGCGTACACCTCGAA | 1 |
| Human_alphaherpesvirus_1---NA.u1.g1 | CG (SEQ ID NO: 377) | |
| Herpesviridae---Simplexvirus--- | ACCATCGAGCTGTACAAG (SEQ ID NO: 378) | 1 |
| Human_alphaherpesvirus_1---NA.u1.g2 | | |
| Orthomyxoviridae---Alphainfluenzavirus--- | gtTAATACGACTCACTATAGGGTCTAATGTCGCAG | 1 |
| Influenza_A_virus---1.u1.g1 | TCTCG (SEQ ID NO: 379) | |
| Orthomyxoviridae---Alphainfluenzavirus--- | TCATTGCCATCATCCATTTC (SEQ ID NO: 380) | 1 |
| Influenza_A_virus---1.u1.g2 | | |
| Paramyxoviridae---Morbillivirus--- | gtTAATACGACTCACTATAGGGACAGCTGCTGAA | 1 |
| Measles_morbillivirus---NA.u1.g1 | GGAATT (SEQ ID NO: 381) | |
| Paramyxoviridae---Morbillivirus--- | CTAGCCGGCTGGATTTTA (SEQ ID NO: 382) | 1 |
| Measles_morbillivirus---NA.u1.g2 | | |
| Parannyxoviridae---Rubulavirus--- | gtTAATACGACTCACTATAGGGATGCTCACCTATC | 1 |
| Munnps_rubulavirus---NA..u1.g1 | ACTGC (SEQ ID NO: 383) | |
| Paramyxoviridae---Rubulavirus--- | AATCTCGTTCGCACTGAT (SEQ ID NO: 384) | 1 |
| Munnps_rubulavirus---NA..u1.g2 | | |
| Retroviridae---Lentivirus--- | gtTAATACGACTCACTATAGGGATGGCCATTGACA | 1 |
| Human_immunodeficiency_virus_1---NA.u1.g1 | GAAGA (SEQ ID NO: 385) | |
| Retroviridae---Lentivirus--- | TGGATTTTCAGGCCCAAT (SEQ ID NO: 386) | 1 |
| Human_immunodeficiency_virus_1---NA.u1.g2 | | |
| Rhabdoviridae---Lyssavirus---Rabies_lyssavirus--- | gtTAATACGACTCACTATAGGGACACCCCTCCTTTT | 1 |
| NA.u1.g1 | GAAC (SEQ ID NO: 387) | |
| Rhabdoviridae---Lyssavirus---Rabies_lyssavirus--- | TGGAGATGAGCCTGATTG (SEQ ID NO: 388) | 1 |
| NA.u1.g2 | | |
| Togaviridae---Alphavirus---Chikungunya_virus--- | gtTAATACGACTCACTATAGGGAATAACGATGAG | 1 |
| NA.u1.g1 | CCCAGG (SEQ ID NO: 389) | |
| Togaviridae---Alphavirus---Chikungunya_virus--- | TCGGTAGTCTTGCACATC (SEQ ID NO: 390) | 1 |
| NA.u1.g2 | | |
| Arenaviridae---Mammarenavirus--- | gtTAATACGACTCACTATAGGGATGGCACTCACAA | 2 |
| Lymphocytic_choriomeningitis_mammarenavirus--- | CAGG (SEQ ID NO: 391) | |
| L.u1.g1 | | |
| Arenaviridae---Mammarenavirus--- | GGATCATGTCAGCACC (SEQ ID NO: 392) | 2 |
| Lymphocytic_choriomeningitis_mammarenavirus--- | | |
| L.u1.g10 | | |
| Arenaviridae---Mammarenavirus--- | GACCATGTAAGCACC (SEQ ID NO: 393) | 2 |
| Lymphocytic_choriomeningitis_mammarenavirus--- | | |
| L.u1.g11 | | |
| Arenaviridae---Mammarenavirus--- | GGGATCATGTTAGCACT (SEQ ID NO: 394) | 2 |
| Lymphocytic_choriomeningitis_mammarenavirus--- | | |
| L.u1.g2 | | |
| Arenaviridae---Mammarenavirus--- | gtTAATACGACTCACTATAGGGTCAGTGCATTGAC | 2 |
| Lymphocytic_choriomeningitis_mammarenavirus--- | GACAG (SEQ ID NO: 395) | |
| L.u1.g3 | | |
| Arenaviridae---Mammarenavirus--- | GGAAGGATCATGTCAGCA (SEQ ID NO: 396) | 2 |
| Lymphocytic_choriomeningitis_mammarenavirus--- | | |
| L.u1.g4 | | |
| Arenaviridae---Mammarenavirus--- | AACAGG (SEQ ID NO: 397) | 2 |
| Lymphocytic_choriomeningitis_mammarenavirus--- | | |
| L.u1.g5 | | |
| Arenaviridae---Mammarenavirus--- | AGGTGTATGATGTTGGTGA (SEQ ID NO: 398) | 2 |
| Lymphocytic_choriomeningitis_mammarenavirus--- | | |
| L.u1.g6 | | |
| Arenaviridae---Mammarenavirus--- | ACAGG (SEQ ID NO: 399) | 2 |
| Lymphocytic_choriomeningitis_mammarenavirus--- | | |
| L.u1.g7 | | |
| Arenaviridae---Mammarenavirus--- | AAGTGTATGATGTTGGTGAT (SEQ ID NO: 400) | 2 |
| Lymphocytic_choriomeningitis_mammarenavirus--- | | |
| L.u1.g8 | | |
| Arenaviridae---Mammarenavirus--- | GGGATCATGTTAGCACC (SEQ ID NO: 401) | 2 |
| Lymphocytic_choriomeningitis_mammarenavirus--- | | |
| L.u1.g9 | | |
| Caliciviridae---Norovirus---Norwalk_virus--- | gtTAATACGACTCACTATAGGGAGCCAATGTTCAG | 2 |
| NA.u1.g1 | ATGGA (SEQ ID NO: 402) | |
| Caliciviridae---Norovirus---Norwalk_virus--- | | 2 |
| NA.u1.g2 | ATTCGACGCCATCTTCAT (SEQ ID NO: 403) | |
| Caliciviridae---Norovirus---Norwalk_virus--- | gtTAATACGACTCACTATAGGGCCATGTTCCGCTG | 2 |
| NA.u1.g3 | GAT (SEQ ID NO: 404) | |
| Caliciviridae---Norovirus---Norwalk_virus--- | gtTAATACGACTCACTATAGGGGATCTGTTCTGCG | 2 |
| NA.u1.g4 | CTGG (SEQ ID NO: 405) | |
| Caliciviridae---Norovirus---Norwalk_virus--- | gtTAATACGACTCACTATAGGGACCCATGTTCAGG | 2 |
| NA.u1.g5 | TGGAT (SEQ ID NO: 406) | |
| Papillomaviridae---Betapapillomavirus--- | gtTAATACGACTCACTATAGGGACAAGGCTTTGGA | 2 |
| Betapapillomavirus_2---NA.u1.g1 | ACCAA (SEQ ID NO: 407) | |
| Papillonnaviridae---Betapapillomavirus--- | | 2 |
| Betapapillomavirus_2---NA.u1.g2 | TTGCAGTGCATTGCG (SEQ ID NO: 408) | |
| Papillomaviridae---Betapapillomavirus--- | gtTAATACGACTCACTATAGGGTAGGCTGTGGAC | 2 |
| Betapapillomavirus_2---NA.u1.g3 | ACA (SEQ ID NO: 409) | |
| Papillomaviridae---Betapapillomavirus--- | TTGTAGTGCACTGCG (SEQ ID NO: 410) | 2 |
| Betapapillomavirus_2---NA.u1.g4 | | |
| Papillomaviridae---Betapapillomavirus--- | gtTAATACGACTCACTATAGGGAGGCTTTGGACAC | 2 |
| Betapapillomavirus_2---NA.u1.g5 | AA (SEQ ID NO: 411) | |
| Papillomaviridae---Betapapillomavirus--- | CTTGCAGTGCATTGC (SEQ ID NO: 412) | 2 |
| Betapapillomavirus_2---NA.u1.g6 | | |
| Papillomaviridae---Betapapillomavirus--- | gtTAATACGACTCACTATAGGGTGGGCTTTGGAG | 2 |
| Betapapillomavirus_2---NA.u1.g7 | ACA (SEQ ID NO: 413) | |
| Phenuiviridae---Phlebovirus---Candiru_phlebovirus- | gtTAATACGACTCACTATAGGGGATCCTGGTGTCT | 2 |
| --L.u1.g1 | GG (SEQ ID NO: 414) | |
| Phenuiviridae---Phlebovirus---Candiru_phlebovirus- | CCTTTCCCAACATGCTGT (SEQ ID NO: 415) | 2 |
| --L.u1.g10 | | |
| Phenuiviridae---Phlebovirus---Candiru_phlebovirus- | gtTAATACGACTCACTATAGGGGCTCATGGTGTCT | 2 |
| --L.u1.g11 | GG (SEQ ID NO: 416) | |
| Phenuiviridae---Phlebovirus---Candiru_phlebovirus- | CCTTTACCTACATGCTGC (SEQ ID NO: 417) | 2 |
| --L.u1.g12 | | |
| Phenuiviridae---Phlebovirus---Candiru_phlebovirus- | GCCCTTTCCCTACATGTT (SEQ ID NO: 418) | 2 |
| --L.u1.g2 | | |
| Phenuiviridae---Phlebovirus---Candiru_phlebovirus- | gtTAATACGACTCACTATAGGGGCTCTTGGTGCCT | 2 |
| --L.u1.g3 | G (SEQ ID NO: 419) | |
| Phenuiviridae---Phlebovirus---Candiru_phlebovirus- | CTGGGCCCACATGTTG (SEQ ID NO: 420) | 2 |
| --L.u1.g4 | | |
| Phenuiviridae---Phlebovirus---Candiru_phlebovirus- | gtTAATACGACTCACTATAGGGGATCGTGGTGTCT | 2 |
| --L.u1.g5 | GG (SEQ ID NO: 421) | |
| Phenuiviridae---Phlebovirus---Candiru_phlebovirus- | GGCACCCACATGTTGT (SEQ ID NO: 422) | 2 |
| --L.u1.g6 | | |
| Phenuiviridae---Phlebovirus---Candiru_phlebovirus- | gtTAATACGACTCACTATAGGGGTTCATGGTGTCA | 2 |
| --L.u1.g7 | GATGG (SEQ ID NO: 423) | |
| Phenuiviridae---Phlebovirus---Candiru_phlebovirus- | CTTTCCCCACATGCTGT (SEQ ID NO: 424) | 2 |
| --L.u1.g8 | | |
| Phenuiviridae---Phlebovirus---Candiru_phlebovirus- | gtTAATACGACTCACTATAGGGGATCTTGGTGCCA | 2 |
| --L.u1.g9 | GATGG (SEQ ID NO: 425) | |
| Caliciviridae---Sapovirus---Sapporo_virus--- | GGDCTHCCMTCWGGSATGCC (SEQ ID NO: 426) | 3 |
| NA.u1.g1 | | |
| Caliciviridae---Sapovirus---Sapporo_virus--- | TAHABRCARTCATCMCCRTA (SEQ ID NO: 427) | 3 |
| NA.u1.g2 | | |
| Retroviridae---Lentivirus--- | TGGCTGGAYTGTACMCA (SEQ ID NO: 428) | 3 |
| Simian_immunodeficiency_virus---NA.u1.g1 | | |
| Retroviridae---Lentivirus--- | TGWCTYTGTGGATTRTAWGG (SEQ ID NO: 429) | 3 |
| Simian_immunodeficiency_virus---NA.u1.g2 | | |
| --Deltavirus---Hepatitis_delta_virus---NA.u1.g1 | gtTAATACGACTCACTATAGGGCCGGCTACTCTTC | 4 |
| | TTGC (SEQ ID NO: 430) | |
| ---Deltavirus---Hepatitis_delta_virus---NA.u1.g2 | CACCGACGAAGGAAGG (SEQ ID NO: 431) | 4 |
| ---Deltavirus---Hepatitis_delta_virus---NA.u1.g3 | gtTAATACGACTCACTATAGGGCCGGCTACTCTTC | 4 |
| | TTTCC (SEQ ID NO: 432) | |
| ---Deltavirus---Hepatitis_delta_virus---NA.u1.g4 | CCACCGAAGAAGGAAGG (SEQ ID NO: 433) | 4 |
| ---Deltavirus---Hepatitis_delta_virus---NA.u1.g5 | gtTAATACGACTCACTATAGGGCCGGCTGTTCTTC | 4 |
| | TTTTC (SEQ ID NO: 434) | |
| ---Deltavirus---Hepatitis_delta_virus---NA.u1.g6 | TTCGACGAACAGAAGACC (SEQ ID NO: 435) | 4 |
| Adenoviridae---Mastadenovirus--- | gtTAATACGACTCACTATAGGGATGGATTCGGGG | 4 |
| Human_mastadenovirus_B---NA.u1.g1 | GAGTAT (SEQ ID NO: 436) | |
| Adenoviridae---Mastadenovirus--- | TGTTTTTGACCCCGATGA (SEQ ID NO: 437) | 4 |
| Human_mastadenovirus_B---NA.u1.g2 | | |
| Adenoviridae---Mastadenovirus--- | gtTAATACGACTCACTATAGGGTAGGTGACGAGA | 4 |
| Human_mastadenovirus_C---NA.u1.g1 | CGC (SEQ ID NO: 438) | |
| Adenoviridae---Mastadenovirus--- | TTTACAGCCAGCACG (SEQ ID NO: 439) | 4 |
| Human_mastadenovirus_C---NA.u1.g2 | | |
| Adenoviridae---Mastadenovirus--- | gtTAATACGACTCACTATAGGGTGCGTTCTCTTCCT | 4 |
| Human_mastadenovirus_D---NA.u1.g1 | TGTT (SEQ ID NO: 440) | |
| Adenoviridae---Mastadenovirus--- | GTAGGAGCCATATACCGC (SEQ ID NO: 441) | 4 |
| Human_mastadenovirus_D---NA.u1.g2 | | |
| Adenoviridae---Mastadenovirus--- | gtTAATACGACTCACTATAGGGCCTGGCCTACAAC | 4 |
| Human_mastadenovirus_E---NA.u1.g1 | TATGG (SEQ ID NO: 442) | |
| Adenoviridae---Mastadenovirus--- | GACCAGTAGACTTGCTCC (SEQ ID NO: 443) | 4 |
| Human_mastadenovirus_E---NA.u1.g2 | | |
| Adenoviridae---Mastadenovirus--- | gtTAATACGACTCACTATAGGGCAGCGCTTGGATT | 4 |
| Human_mastadenovirus_F---NA.u1.g1 | ACATG (SEQ ID NO: 444) | |
| Adenoviridae---Mastadenovirus--- | GTGTGTACCTTTGGTGGA (SEQ ID NO: 445) | 4 |
| Human_mastadenovirus_F---NA.u1.g2 | | |
| Anelloviridae---Betatorquevirus---TTV- | gtTAATACGACTCACTATAGGGGAACTTGGGCGG | 4 |
| like_mini_virus---NA.u1.g1 | GTG (SEQ ID NO: 446) | |
| Anelloviridae---Betatorquevirus---TTV- | CGCCAGACTGATCTAGC (SEQ ID NO: 447) | 4 |
| like_mini_virus---NA.u1.g2 | | |
| Anelloviridae---Betatorquevirus---TTV- | gtTAATACGACTCACTATAGGGTGATCTTGGGCGG | 4 |
| like_mni_virus---NA.u1.g3 | GAG (SEQ ID NO: 448) | |
| Anelloviridae---Betatorquevirus---TTV- | CACCAGACTGAACTAGCC (SEQ ID NO: 449) | 4 |
| like_mini_virus---NA.u1.g4 | | |
| Anelloviridae---Gyrovirus---Avian_gyrovirus_2--- | gtTAATACGACTCACTATAGGGTATGCGCGTAGAA | 4 |
| NA.u1.g1 | GATCC (SEQ ID NO: 450) | |
| Anelloviridae---Gyrovirus---Avian_gyrovirus_2--- | GCCTCCGGAATGAATACA (SEQ ID NO: 451) | 4 |
| NA.u1.g2 | | |
| Anelloviridae---Gyrovirus---Chicken_anemia_virus-- | gtTAATACGACTCACTATAGGGGAACGCTCTCCAA | 4 |
| -NA.u1.g1 | GAAGA (SEQ ID NO: 452) | |
| Anelloviridae---Gyrovirus---Chicken_anemia_virus-- | TTCCAGCGATACCAATCC (SEQ ID NO: 453) | 4 |
| -NA.u1.g2 | | |
| Anelloviridae---Iotatorquevirus--- | gtTAATACGACTCACTATAGGGGCTCAAGTCCTCA | 4 |
| Torque_teno_sus_virus_1a---NA.u1.g1 | TTTGC (SEQ ID NO: 454) | |
| Anelloviridae---Iotatorquevirus--- | CTCAGCCATTCGGAA (SEQ ID NO: 455) | 4 |
| Torque_teno_sus_virus_1a---NA.u1.g2 | | |
| Anelloviridae---Iotatorquevirus--- | gtTAATACGACTCACTATAGGGAGCTCCGGTCATA | 4 |
| Torque_teno_sus_virus_1b---NA.u1.g1 | CAATG (SEQ ID NO: 456) | |
| Anelloviridae---Iotatorquevirus--- | GTACGGAACCAGTGTCC (SEQ ID NO: 457) | 4 |
| Torque_teno_sus_virus_1b---NA.u1.g2 | | |
| Anelloviridae------ | gtTAATACGACTCACTATAGGGGCTWCAGTAAGA | 4 |
| Torque_teno_Leptonychotes_weddellii_virus-1--- | TATTACCCCT (SEQ ID NO: 458) | |
| NA.u1.g1 | | |
| Anelloviridae------ | GYTCCCAACCTCKAAC (SEQ ID NO: 459) | 4 |
| Torque_teno_Leptonychotes_weddellii_virus-1--- | | |
| NA.u1.g2 | | |
| Anelloviridae------ | gtTAATACGACTCACTATAGGGGAGTTTTTGCTGC | 4 |
| Torque_teno_Leptonychotes_weddellii_virus-2--- | TGGAG (SEQ ID NO: 460) | |
| NA.u1.g1 | | |
| Anelloviridae------ | GTTTTGCTGTACGGATCG (SEQ ID NO: 1065) | 4 |
| Torque_teno_Leptonychotes_weddellii_virus-2--- | | |
| NA.u1.g2 | | |
| Arenaviridae---Arenavirus---Mopeia_Arenaviridae-- | gtTAATACGACTCACTATAGGGACGTTTGGTGGA | 5 |
| -Mammarenavirus---Lassa_mammarenavirus--- | GTGATT (SEQ ID NO: 1066) | |
| S_virus_reassortant_29---L.u1.g1 | | |
| Arenaviridae---Arenavirus---Mopeia_Arenaviridae-- | TTACGTGTCCACTTTGCT (SEQ ID NO: 1067) | 5 |
| -Mammarenavirus---Lassa_mammarenavirus--- | | |
| S_virus_reassortant_29---L.u1.g2 | | |
| Arenaviridae---Mammarenavirus--- | gtTAATACGACTCACTATAGGGTGAACAGGACAA | 5 |
| Argentinian_mammarenavirus---L.u1.g1 | GTCACC (SEQ ID NO: 1068) | |
| Arenaviridae---Mammarenavirus--- | CTCAGAAGCTGTGGGTAG (SEQ ID NO: 1069) | 5 |
| Argentinian_mammarenavirus---L.u1.g2 | | |
| Arenaviridae---Mammarenavirus--- | gtTAATACGACTCACTATAGGGATCTGATGAGATG | 5 |
| Cali_mammarenavirus---S.u1.g1 | TGGCC (SEQ ID NO: 1070) | |
| Arenaviridae---Mammarenavirus--- | GGTGAGATTGTGCCTTCT (SEQ ID NO: 1071) | 5 |
| Cali_mammarenavirus---S.u1.g2 | | |
| Arenaviridae---Mammarenavirus--- | gtTAATACGACTCACTATAGGGGACACCATTAGCC | 5 |
| Guanarito_mammarenavirus---L.u1.g1 | ACACA (SEQ ID NO: 1072) | |
| Arenaviridae---Mammarenavirus--- | TCATGGGTGAAGAGACAC (SEQ ID NO: 1073) | 5 |
| Guanarito_mammarenavirus---L.u1.g2 | | |
| Arenaviridae---Mammarenavirus--- | gtTAATACGACTCACTATAGGGCAACACCATTAGC | 5 |
| Guanarito_mammarenavirus---L.u1.g3 | TACACA (SEQ ID NO: 1074) | |
| Arenaviridae---Mammarenavirus--- | TCATGGGTGAGGCAC (SEQ ID NO: 461) | 5 |
| Guanarito_mammarenavirus---L.u1.g4 | | |
| Arenaviridae---Mammarenavirus--- | gtTAATACGACTCACTATAGGGGGGCGGTGGGTC | 5 |
| Lassa_mammarenavirus---S.u1.g1 | (SEQ ID NO: 462) | |
| Arenaviridae---Mammarenavirus--- | ATAATGTATGATGCAGCTGT (SEQ ID NO: 463) | 5 |
| Lassa_mammarenavirus---S.u1.g2 | | |
| Arenaviridae---Mmmarenavirus--- | gtTAATACGACTCACTATAGGGCTATTGGCGGTGG | 5 |
| Lassa_mammarenavirus---S.u1.g3 | GTC (SEQ ID NO: 464) | |
| Arenaviridae---Mammarenavirus--- | CATGTTTGATGCAGCAGT (SEQ ID NO: 465) | 5 |
| Lassa_mammarenavirus---S.u1.g4 | | |
| Arenaviridae---Mammarenavirus--- | gtTAATACGACTCACTATAGGGTGACAATTGTGTG | 5 |
| Machupo_mammarenavirus---L.u1.g1 | GGTGT (SEQ ID NO: 466) | |
| Arenaviridae---Mammarenavirus--- | GTCATGGGTGAAGCAC (SEQ ID NO: 467) | 5 |
| Machupo_mammarenavirus---L.u1.g2 | | |
| Arenaviridae---Mammarenavirus--- | gtTAATACGACTCACTATAGGGATGCTCCCTCTTCC | 5 |
| Whitewater_Arroyo_mammarenavirus---S.u1.g1 | A (SEQ ID NO: 468) | |
| Arenaviridae---Mammarenavirus--- | CCATGGTCTTTACTGCAC (SEQ ID NO: 469) | 5 |
| Whitewater_Arroyo_mammarenavirus---S.u1.g2 | | |
| Arenaviridae---Mammarenavirus--- | gtTAATACGACTCACTATAGGGGGTGCTCTCTCTT | 5 |
| Whitewater_Arroyo_mammarenavirus---S.u1.g3 | CC (SEQ ID NO: 470) | |
| Arenaviridae---Mammarenavirus--- | TCAATGGTTTTCACTGCAC (SEQ ID NO: 471) | 5 |
| Whitewater_Arroyo_mammarenavirus---S.u1.g4 | | |
| Astroviridae---Mamastrovirus---Mamastrovirus_1--- | gtTAATACGACTCACTATAGGGTCCATGGGAAGCT | 5 |
| NA.u1.g1 | CCTAT (SEQ ID NO: 472) | |
| Astroviridae---Mamastrovirus---Mamastrovirus_1--- | GAGTCACGAAGCTGCTT (SEQ ID NO: 473) | 5 |
| NA.u1.g2 | | |
| Coronaviridae---Alphacoronavirus--- | gtTAATACGACTCACTATAGGGAGTGTCCGTGATG | 5 |
| Human_coronavirus_229E---NA..u1.g1 | GT (SEQ ID NO: 474) | |
| Coronaviridae---Alphacoronavirus--- | GCTCTACCGCTAACACTT (SEQ ID NO: 475) | 5 |
| Human_coronavirus_229E---NA..u1.g2 | | |
| Coronaviridae---Alphacoronavirus--- | gtTAATACGACTCACTATAGGGTGGTGAATGGAA | 5 |
| Human_coronavirus_NL63---NA.u1.g1 | TGCTGT (SEQ ID NO: 476) | |
| Coronaviridae---Alphacoronavirus--- | CACCAACACTCCAACTCT (SEQ ID NO: 477) | 5 |
| Human_coronavirus_NL63---NA.u1.g2 | | |
| Coronaviridae---Betacoronavirus--- | gtTAATACGACTCACTATAGGGGAAGTCAGATGA | 5 |
| Human_coronavirus_HKU1---NA..u1.g1 | GGGTGG (SEQ ID NO: 478) | |
| Coronaviridae---Betacoronavirus--- | ACATGCCATTCTTGTCCA (SEQ ID NO: 479) | 5 |
| Human_coronavirus_HKU1---NA..u1.g2 | | |
| Coronaviridae---Betacoronavirus--- | gtTAATACGACTCACTATAGGGGTCTGCATGTTGT | 5 |
| Severe_acute_respiratory_syndronne- | TGGAC (SEQ ID NO: 480) | |
| related_coronavirus---NA.u1.g1 | | |
| Coronaviridae---Betacoronavirus--- | CTGCTGACAACAATGGTG (SEQ ID NO: 481) | 5 |
| Severe_acute_respiratory_syndronne- | | |
| related_coronavirus---NA.u1.g2 | | |
| Filoviridae---Ebolavirus---Reston_ebolavirus--- | gtTAATACGACTCACTATAGGGAATTCAGTTGCTC | 6 |
| NA.u1.g1 | AGGCT (SEQ ID NO: 482) | |
| Filoviridae---Ebolavirus---Reston_ebolavirus--- | GTCTTACTCCTTGGTCGG (SEQ ID NO: 483) | 6 |
| NA.u1.g2 | | |
| Filoviridae---Marburgvirus--- | gtTAATACGACTCACTATAGGGTTCATCAACTGAG | 6 |
| Marburg_marburgvirus---NA.u1.g1 | GGTCG (SEQ ID NO: 484) | |
| Filoviridae---Marburgvirus--- | TACTGAGAACATGTCGGC (SEQ ID NO: 485) | 6 |
| Marburg_marburgvirus---NA.u1.g2 | | |
| Flaviviridae---Flavivirus---Bagaza_virus---NA.u1.g1 | gtTAATACGACTCACTATAGGGICTGGATCTGATG | 6 |
| | GACCA (SEQ ID NO: 486) | |
| Flaviviridae---Flavivirus---Bagaza_virus---NA.u1.g2 | TTGTCCCCGATGATGATG (SEQ ID NO: 487) | 6 |
| Flaviviridae---Flavivirus---Culex_flavivirus--- | gtTAATACGACTCACTATAGGGGCTGTGGGAATC | 6 |
| NA.u1.g1 | GACATA (SEQ ID NO: 488) | |
| Flaviviridae---Flavivirus---Culex_flavivirus--- | AGTTCAGCAGTACCATCG (SEQ ID NO: 489) | 6 |
| NA.u1.g2 | | |
| Flaviviridae---Flavivirus--- | gtTAATACGACTCACTATAGGGTGTGGAAGACCG | 6 |
| Japanese_encephalitis_virus---NA.u1.g1 | CAT (SEQ ID NO: 490) | |
| Flaviviridae---Flavivirus--- | ACTCCTGGTTTTGTCTGG (SEQ ID NO: 491) | 6 |
| Japanese_encephalitis_virus---NA.u1.g2 | | |
| Flaviviridae---Flavivirus--- | gtTAATACGACTCACTATAGGGTCCAGTGCATGCT | 6 |
| Kyasanur_Forest_disease_virus---NA.u1.g1 | CATAG (SEQ ID NO: 492) | |
| Flaviviridae---Flavivirus--- | CCACACAACTGCACA (SEQ ID NO: 493) | 6 |
| Kyasanur_Forest_disease_virus---NA.u1.g2 | | |
| Flaviviridae---Flavivirus--- | gtTAATACGACTCACTATAGGGAATATGCTACGCG | 6 |
| Murray_Valley_encephalitis_virus---NA.u1.g1 | GC (SEQ ID NO: 494) | |
| Flaviviridae---Flavivirus--- | GCAAGTGCTGTCCTG (SEQ ID NO: 495) | 6 |
| Murray_Valley_encephalitis_virus---NA.u1.g2 | | |
| Flaviviridae---Flavivirus---Powassan_virus--- | gtTAATACGACTCACTATAGGGTTGGGGCAAGTC | 6 |
| NA.u1.g1 | AATCTT (SEQ ID NO: 496) | |
| Flaviviridae---Flavivirus---Powassan_virus--- | AACACTCCTGTTGCTCTC (SEQ ID NO: 497) | 6 |
| NA.u1.g2 | | |
| Flaviviridae---Flavivirus--- | gtTAATACGACTCACTATAGGGCGGGGTTGAAGA | 6 |
| Saint_Louis_encephalitis_virus---NA.u1.g1 | GGATAC (SEQ ID NO: 498) | |
| Flaviviridae---Flavivirus--- | ATCTACAGCCCTCCATCT (SEQ ID NO: 499) | 6 |
| Saint_Louis_encephalitis_virus---NA.u1.g2 | | |
| Flaviviridae---Flavivirus---Tennbusu_virus---NA.u1.g1 | gtTAATACGACTCACTATAGGGAGGGAGTGAATG | 6 |
| | GTGTTG (SEQ ID NO: 500) | |
| Flaviviridae---Flavivirus---Tennbusu_virus---NA.u1.g2 | AATTCCGTAGCCTCCATG (SEQ ID NO: 501) | 6 |
| Flaviviridae---Flavivirus---Tick- | gtTAATACGACTCACTATAGGGAGAACAAGAGCT | 6 |
| borne_encephalitis_virus---NA.u1.g1 | GGGGAT (SEQ ID NO: 502) | |
| Flaviviridae---Flavivirus---Tick- | CGGTCTCTTTCGACACTC (SEQ ID NO: 503) | 6 |
| borne_encephalitis_virus---NA.u1.g2 | | |
| Flaviviridae---Flavivirus---Usutu_virus---NA.u1.g1 | gtTAATACGACTCACTATAGGGTGTCTCCAACTGT | 6 |
| | CCAAC (SEQ ID NO: 504) | |
| Flaviviridae---Flavivirus---Usutu_virus---NA.u1.g2 | TGGCACACGTGTCTATAC (SEQ ID NO: 505) | 6 |
| Flaviviridae---Flavivirus---West_Nile_virus--- | gtTAATACGACTCACTATAGGGAAGTCTGGAAGC | 6 |
| NA..u1.g1 | AGCATT (SEQ ID NO: 506) | |
| Flaviviridae---Flavivirus---West_Nile_virus--- | CCAAGCTGTGTCTCCTAG (SEQ ID NO: 507) | 6 |
| NA..u1.g2 | | |
| Flaviviridae---Flavivirus---Yellow_fever_virus--- | gtTAATACGACTCACTATAGGGTTGGTCTGCTCGA | 6 |
| NA.u1.g1 | GT (SEQ ID NO: 508) | |
| Flaviviridae---Flavivirus---Yellow_fever_virus--- | GTACCATATTGACGCCCA (SEQ ID NO: 509) | 6 |
| NA.u1.g2 | | |
| Flaviviridae---Hepacivirus---Hepacivirus_C--- | gtTAATACGACTCACTATAGGGTGAGCACACTTCC | 6 |
| NA.u1.g1 | TCC (SEQ ID NO: 510) | |
| Flaviviridae---Hepacivirus---Hepacivirus_C--- | GCGCGGCAACAAGTA (SEQ ID NO: 511) | 6 |
| NA.u1.g2 | | |
| Flaviviridae---Pegivirus---Pegivirus_A---NA.u1.g1 | gtTAATACGACTCACTATAGGGGTACGGGTTGGA | 7 |
| | GCCT (SEQ ID NO: 512) | |
| Flaviviridae---Pegivirus---Pegivirus_A---NA.u1.g2 | GGCTTCTCCGATGTCAG (SEQ ID NO: 513) | 7 |
| Flaviviridae---Pegivirus---Pegivirus_A---NA.u1.g3 | gtTAATACGACTCACTATAGGGGGTATGGAATGG | 7 |
| | AACCTGA (SEQ ID NO: 514) | |
| Flaviviridae---Pegivirus---Pegivirus_A---NA.u1.g4 | GGCTTCACCAATGTCAG (SEQ ID NO: 515) | 7 |
| Flaviviridae---Pegivirus---Pegivirus_C---NA.u1.g1 | gtTAATACGACTCACTATAGGGATGTCAGCTGGGC | 7 |
| | A (SEQ ID NO: 516) | |
| Flaviviridae---Pegivirus---Pegivirus_C---NA.u1.g2 | CATTCTGGGTCGTCGG (SEQ ID NO: 517) | 7 |
| Flaviviridae---Pegivirus---Pegivirus_C---NA.u1.g3 | gtTAATACGACTCACTATAGGGTGTTAGCTGGGCA | 7 |
| | AC (SEQ ID NO: 518) | |
| Flaviviridae---Pegivirus---Pegivirus_C---NA.u1.g4 | CATTGGGGGTCATCCG (SEQ ID NO: 519) | 7 |
| Flaviviridae---Pegivirus---Pegivirus_H---NA.u1.g1 | gtTAATACGACTCACTATAGGGGTGGCCATCAAGC | 7 |
| | TATCT (SEQ ID NO: 520) | |
| Flaviviridae---Pegivirus---Pegivirus_H---NA.u1.g2 | AACTCCACCAACCAAGAG (SEQ ID NO: 521) | 7 |
| Hantaviridae---Orthohantavirus--- | gtTAATACGACTCACTATAGGGTGGCTACACCAGT | 7 |
| Andes_orthohantavirus---S.u1.g1 | TG (SEQ ID NO: 522) | |
| Hantaviridae---Orthohantavirus--- | CATCCAGGACATTCCCA (SEQ ID NO: 523) | 7 |
| Andes_orthohantavirus---S.u1.g2 | | |
| Hantaviridae---Orthohantavirus---Dobrava- | gtTAATACGACTCACTATAGGGCTTTCCAGTTGGG | 7 |
| Belgrade_orthohantavirus---L.u1.g1 | TCACT (SEQ ID NO: 524) | |
| Hantaviridae---Orthohantavirus---Dobrava- | TCTGACCAGTCATGCTTT (SEQ ID NO: 525) | 7 |
| Belgrade_orthohantavirus---L.u1.g2 | | |
| Hantaviridae---Orthohantavirus--- | gtTAATACGACTCACTATAGGGCACAATGGCCCAG | 7 |
| Hantaan_orthohantavirus---L.u1.g1 | TAGAA (SEQ ID NO: 526) | |
| Hantaviridae---Orthohantavirus--- | ACATGGCTTCTAGTGCAG (SEQ ID NO: 527) | 7 |
| Hantaan_orthohantavirus---L.u1.g2 | | |
| Hantaviridae---Orthohantavirus--- | gtTAATACGACTCACTATAGGGGGCACAATAGGA | 7 |
| Innjin_orthohantavirus---L.u1.g1 | GCAGTA (SEQ ID NO: 528) | |
| Hantaviridae---Orthohantavirus--- | CAATTAGGTCATGGCGGA (SEQ ID NO: 529) | 7 |
| Innjin_orthohantavirus---L.u1.g2 | | |
| Hantaviridae---Orthohantavirus--- | gtTAATACGACTCACTATAGGGAGAGCACTAATCA | 7 |
| Nova_orthohantavirus---S.u1.g1 | CAGCA (SEQ ID NO: 530) | |
| Hantaviridae---Orthohantavirus--- | GCAGCTTCCTTTGCTTC (SEQ ID NO: 531) | 7 |
| Nova_orthohantavirus---S.u1.g2 | | |
| Hantaviridae---Orthohantavirus--- | gtTAATACGACTCACTATAGGGAGAGCACTAATCA | 7 |
| Nova_orthohantavirus---S.u1.g3 | CAGCA (SEQ ID NO: 532) | |
| Hantaviridae---Orthohantavirus--- | CAGCCTCCTTTGCCTC (SEQ ID NO: 533) | 7 |
| Nova_orthohantavirus---S.u1.g4 | | |
| Hantaviridae---Orthohantavirus--- | gtTAATACGACTCACTATAGGGAGAGGATATAAC | 7 |
| Puunnala_orthohantavirus---S.u1.g1 | CCGCCA (SEQ ID NO: 534) | |
| Hantaviridae---Orthohantavirus--- | CTGACACTGTTTGTTGCC (SEQ ID NO: 535) | 7 |
| Puunnala_orthohantavirus---S.u1.g2 | | |
| Hantaviridae---Orthohantavirus--- | gtTAATACGACTCACTATAGGGCACGTCTCAGGTG | 7 |
| Seoul_orthohantavirus---L.u1.g1 | GT (SEQ ID NO: 536) | |
| Hantaviridae---Orthohantavirus--- | CTTGTACTTGGCCTGACA (SEQ ID NO: 537) | 7 |
| Seoul_orthohantavirus---L.u1.g2 | | |
| Hantaviridae---Orthohantavirus--- | gtTAATACGACTCACTATAGGGACATTACAGAGCA | 7 |
| Sin_Nonnbre_orthohantavirus---S.u1.g1 | GACGG (SEQ ID NO: 538) | |
| Hantaviridae---Orthohantavirus--- | AGGTTCAATCCCTGTTGG (SEQ ID NO: 539) | 7 |
| Sin_Nonnbre_orthohantavirus---S.u1.g2 | | |
| Hantaviridae---Orthohantavirus--- | gtTAATACGACTCACTATAGGGAACCCTGAGAAG | 7 |
| Thottapalayann_orthohantavirus---S.u1.g1 | GCA (SEQ ID NO: 540) | |
| Hantaviridae---Orthohantavirus--- | TAGACTGCTGCTGAATGG (SEQ ID NO: 541) | 7 |
| Thottapalayann_orthohantavirus---S.u1.g2 | | |
| Hantaviridae---Orthohantavirus--- | gtTAATACGACTCACTATAGGGCGACCCGGATGAT | 7 |
| Tula_orthohantavirus---S.u1.g1 | GTTAA (SEQ ID NO: 542) | |
| Hantaviridae---Orthohantavirus--- | ACAGGCTTTTCACCCATT (SEQ ID NO: 543) | 7 |
| Tula_orthohantavirus---S.u1.g2 | | |
| Hepadnaviridae---Orthohepadnavirus--- | gtTAATACGACTCACTATAGGGCACCTGTATTCCC | 8 |
| Hepatitis_B_virus---NA.u1.g1 | ATCCC (SEQ ID NO: 544) | |
| Hepadnaviridae---Orthohepadnavirus--- | AACTGAGCCAGGAGC (SEQ ID NO: 545) | 8 |
| Hepatitis_B_virus---NA.u1.g2 | | |
| Hepeviridae---Orthohepevirus---Orthohepevirus_A- | gtTAATACGACTCACTATAGGGTGCCTATGCTGCC | 8 |
| --NA.u1.g1 | CG (SEQ ID NO: 546) | |
| Hepeviridae---Orthohepevirus---Orthohepevirus_A- | GCGAAGGGCTGAGAATC (SEQ ID NO: 547) | 8 |
| --NA.u1.g2 | | |
| Herpesviridae---Cytomegalovirus--- | gtTAATACGACTCACTATAGGGAAGAGGTTTCAA | 8 |
| Human_betaherpesvirus_5---NA.u1.g1 | GTGCGA (SEQ ID NO: 548) | |
| Herpesviridae---Cytomegalovirus--- | TCTTGGACCACAGTTGTC (SEQ ID NO: 549) | 8 |
| Human_betaherpesvirus_5---NA.u1.g2 | | |
| Herpesviridae---Lymphocryptovirus--- | gtTAATACGACTCACTATAGGGTGTCTGTGGTTGT | 8 |
| Human_gannnnaherpesvirus_4---NA.u1.g1 | CTTCC (SEQ ID NO: 550) | |
| Herpesviridae---Lymphocryptovirus--- | GAACTGCGGGATAATGGA (SEQ ID NO: 551) | 8 |
| Human_gannnnaherpesvirus_4---NA.u1.g2 | | |
| Herpesviridae---Rhadinovirus--- | gtTAATACGACTCACTATAGGGAGCCATTATACAC | 8 |
| Human_gammaherpesvirus_8---NA.u1.g1 | ACGGG (SEQ ID NO: 552) | |
| Herpesviridae---Rhadinovirus--- | GGGAAGTTGTGTGTCAGA (SEQ ID NO: 553) | 8 |
| Human_gammaherpesvirus_8---NA.u1.g2 | | |
| Herpesviridae---Simplexvirus--- | gtTAATACGACTCACTATAGGGTGAAGGCAGAGA | 8 |
| Human_alphaherpesvirus_2---NA.u1.g1 | CGT (SEQ ID NO: 554) | |
| Herpesviridae---Simplexvirus--- | GAGTTGCTCCTGGAGTAC (SEQ ID NO: 555) | 8 |
| Human_alphaherpesvirus_2---NA.u1.g2 | | |
| Herpesviridae---Varicellovirus--- | gtTAATACGACTCACTATAGGGTCCTTGGTTGGTT | 8 |
| Human_alphaherpesvirus_3---NA.u1.g1 | TTGGT (SEQ ID NO: 556) | |
| Herpesviridae---Varicellovirus--- | TACATTCGGATTCTGGCC (SEQ ID NO: 557) | 8 |
| Human_alphaherpesvirus_3---NA.u1.g2 | | |
| Nairoviridae---Orthonairovirus---Crimean- | gtTAATACGACTCACTATAGGGCTGAATCTGTGGA | 8 |
| Congo_hemorrhagic_fever_orthonairovirus--- | GGCAG (SEQ ID NO: 558) | |
| L.u1.g1 | | |
| Nairoviridae---Orthonairovirus---Crimean- | CGCTCTATTGAATGCACC (SEQ ID NO: 559) | 8 |
| Congo_hemorrhagic_fever_orthonairovirus--- | | |
| L.u1.g2 | | |
| Nairoviridae---Orthonairovirus--- | gtTAATACGACTCACTATAGGGCCTTGAACTAGCC | 8 |
| Nairobi_sheep_disease_orthonairovirus---S.u1.g1 | AAGCA (SEQ ID NO: 560) | |
| Nairoviridae---Orthonairovirus--- | CTGTGAGACTGTCGG (SEQ ID NO: 561) | 8 |
| Nairobi_sheep_disease_orthonairovirus---S.u1.g2 | | |
| Orthomyxoviridae---Betainfluenzavirus--- | gtTAATACGACTCACTATAGGGCAGGCAGCAATTT | 8 |
| Influenza_B_virus---1.u1.g1 | CAACA (SEQ ID NO: 562) | |
| Orthomyxoviridae---Betainfluenzavirus--- | GTTCTGATCACGGTGTCT (SEQ ID NO: 563) | 8 |
| Influenza_B_virus---1.u1.g2 | | |
| Orthomyxoviridae---Gannnnainfluenzavirus--- | gtTAATACGACTCACTATAGGGTCTGCTTTAGGAG | 8 |
| Influenza_C_virus---1.u1.g1 | GACCA (SEQ ID NO: 564) | |
| Orthomyxoviridae---Gannnnainfluenzavirus--- | TTGTACTGCTCTGACACC (SEQ ID NO: 565) | 8 |
| Influenza_C_virus---1.u1.g2 | | |
| Papillonnaviridae---Alphapapillomavirus--- | gtTAATACGACTCACTATAGGGAGTGGGTATGGC | 8 |
| Alphapapillomavirus_4---NA.u1.g1 | AATACG (SEQ ID NO: 566) | |
| Papillonnaviridae---Alphapapillomavirus--- | GTTAGATCTGCCTCTCCG (SEQ ID NO: 567) | 8 |
| Alphapapillomavirus_4---NA.u1.g2 | | |
| Papillonnaviridae---Alphapapillomavirus--- | gtTAATACGACTCACTATAGGGTCCAGATTAGATT | 8 |
| Alphapapillomavirus_7---NA.u1.g1 | TGCACG (SEQ ID NO: 568) | |
| Papillonnaviridae---Alphapapillomavirus--- | ACACATTTCGTTGGGA (SEQ ID NO: 569) | 8 |
| Alphapapillomavirus_7---NA.u1.g2 | | |
| Papillonnaviridae---Alphapapillomavirus--- | gtTAATACGACTCACTATAGGGGCAGATTAGACTT | 8 |
| Alphapapillomavirus_7---NA.u1.g3 | GCAGC (SEQ ID NO: 570) | |
| Papillonnaviridae---Alphapapillomavirus--- | CGCACTTCGTTCCG (SEQ ID NO: 571) | 8 |
| Alphapapillomavirus_7---NA.u1.g4 | | |
| Papillonnaviridae---Alphapapillomavirus--- | gtTAATACGACTCACTATAGGGTACAGACCTACGT | 8 |
| Alphapapillomavirus_9---NA.u1.g1 | GACCA (SEQ ID NO: 572) | |
| Papillonnaviridae---Alphapapillomavirus--- | AATCCCATTTCTCTGGCC (SEQ ID NO: 573) | 8 |
| Alphapapillomavirus_9---NA.u1.g2 | | |
| Paramyxoviridae---Morbillivirus--- | gtTAATACGACTCACTATAGGGGGGGCATCTATCA | 9 |
| Canine_morbillivirus---NA.u1.g1 | AGCAT (SEQ ID NO: 574) | |
| Paramyxoviridae---Morbillivirus--- | GCTCTGGGTTAATGTCGA (SEQ ID NO: 575) | 9 |
| Canine_morbillivirus---NA.u1.g2 | | |
| Paramyxoviridae---Morbillivirus--- | gtTAATACGACTCACTATAGGGAGAGGCAACAGC | 9 |
| Rinderpest_morbillivirus---NA.u1.g1 | TGT (SEQ ID NO: 576) | |
| Paramyxoviridae---Morbillivirus--- | ACCAGGATAGAGTCAGCA (SEQ ID NO: 577) | 9 |
| Rinderpest_morbillivirus---NA.u1.g2 | | |
| Papillonnaviridae---Betapapillomavirus--- | gtTAATACGACTCACTATAGGGTGAACTTACTGAC | 9 |
| Betapapillomavirus_1---NA.u1.g1 | CGC (SEQ ID NO: 578) | |
| Papillonnaviridae---Betapapillomavirus--- | CACTGCGCTCGTTG (SEQ ID NO: 579) | 9 |
| Betapapillomavirus_1---NA.u1.g2 | | |
| Papillonnaviridae---Betapapillomavirus--- | gtTAATACGACTCACTATAGGGTGAGTTAACTGAC | 9 |
| Betapapillomavirus_1---NA.u1.g3 | CGC (SEQ ID NO: 580) | |
| Papillonnaviridae---Betapapillomavirus--- | TCGCGTTTTGTCAGC (SEQ ID NO: 581) | 9 |
| Betapapillomavirus_1---NA.u1.g4 | | |
| Papillonnaviridae---Betapapillomavirus--- | gtTAATACGACTCACTATAGGGCGAACTAACTGAC | 9 |
| Betapapillomavirus_1---NA.u1.g5 | CGC (SEQ ID NO: 582) | |
| Papillonnaviridae---Betapapillomavirus--- | ATTGCGCTCGCTGA (SEQ ID NO: 583) | 9 |
| Betapapillomavirus_1---NA.u1.g6 | | |
| Paramnyxoviridae---Avulavirus---Avian_avulavirus_1- | gtTAATACGACTCACTATAGGGGAGTCACAACCAT | 9 |
| --NA.u1.g1 | CAGCT (SEQ ID NO: 584) | |
| Paramyxoviridae---Avulavirus---Avian_avulavirus_1- | TGTGATAATGCCTCCATCA (SEQ ID NO: 585) | 9 |
| --NA.u1.g2 | | |
| Paramyxoviridae---Avulavirus---Avian_avulavirus_1- | gtTAATACGACTCACTATAGGGIGICACCACAATC | 9 |
| --NA.u1.g3 | AGCTG (SEQ ID NO: 586) | |
| Paramyxoviridae---Avulavirus---Avian_avulavirus_1- | GTGATATCGCCTCCATCA (SEQ ID NO: 587) | 9 |
| --NA.u1.g4 | | |
| Paramyxoviridae---Avulavirus---Avian_avulavirus_4- | gtTAATACGACTCACTATAGGGAAGGAACTCCAAC | 9 |
| --NA.u1.g1 | ACCAG (SEQ ID NO: 588) | |
| Paramyxoviridae---Avulavirus---Avian_avulavirus_4- | TGGGGTGGAAGTTGT (SEQ ID NO: 589) | 9 |
| --NA.u1.g2 | | |
| Paramyxoviridae---Avulavirus---Avian_avulavirus_6- | gtTAATACGACTCACTATAGGGATCGTGAGGGGG | 9 |
| --NA.u1.g1 | AAG (SEQ ID NO: 590) | |
| Paramyxoviridae---Avulavirus---Avian_avulavirus_6- | GTGAACACTGACGACATC (SEQ ID NO: 591) | 9 |
| --NA.u1.g2 | | |
| Paramyxoviridae---Henipavirus--- | gtTAATACGACTCACTATAGGGACTACTCCCGAGG | 9 |
| Hendra_henipavirus---NA.u1.g1 | ACAAT (SEQ ID NO: 592) | |
| Paramyxoviridae---Henipavirus--- | CTGCGTACATCAGGAGTT (SEQ ID NO: 593) | 9 |
| Hendra_henipavirus---NA.u1.g2 | | |
| Paramyxoviridae---Henipavirus--- | gtTAATACGACTCACTATAGGGTTTTGCCCCTGGA | 9 |
| Nipah_henipavirus---NA.u1.g1 | GG (SEQ ID NO: 594) | |
| Paramyxoviridae---Henipavirus--- | GGCTCAAGATAACCACGA (SEQ ID NO: 595) | 9 |
| Nipah_henipavirus---NA.u1.g2 | | |
| Paramyxoviridae---Morbillivirus--- | gtTAATACGACTCACTATAGGGAGCTGGTAATCCT | 9 |
| Feline_morbillivirus---NA.u1.g1 | GGAGA (SEQ ID NO: 596) | |
| Paramyxoviridae---Morbillivirus--- | TGGTGGGTTCTCTCC (SEQ ID NO: 597) | 9 |
| Feline_morbillivirus---NA.u1.g2 | | |
| Paramyxoviridae---Morbillivirus--- | gtTAATACGACTCACTATAGGGACGTGGGCAACTT | 9 |
| Snnall_runninant_morbillivirus---NA.u1.g1 | TAGAA (SEQ ID NO: 598) | |
| Paramyxoviridae---Morbillivirus--- | CTCCCAGGGCAACTA (SEQ ID NO: 599) | 9 |
| Snnall_runninant_morbillivirus---NA.u1.g2 | | |
| Paramyxoviridae---Respirovirus--- | gtTAATACGACTCACTATAGGGGAGGACACAGAA | 9 |
| Bovine_respirovirus_3---NA.u1.g1 | GAGAGC (SEQ ID NO: 600) | |
| Paramyxoviridae---Respirovirus--- | TGCAGATTGGATTACACCA (SEQ ID NO: 601) | 9 |
| Bovine_respirovirus_3---NA.u1.g2 | | |
| Paramyxoviridae---Respirovirus--- | gtTAATACGACTCACTATAGGGTGCAGGGATAGG | 10 |
| Human_respirovirus_1---NA.u1.g1 | AGGAAT (SEQ ID NO: 602) | |
| Paramyxoviridae---Respirovirus--- | ATCCACTGTGAAGGTTGG (SEQ ID NO: 603) | 10 |
| Human_respirovirus_1---NA.u1.g2 | | |
| Paramyxoviridae---Respirovirus--- | gtTAATACGACTCACTATAGGGTGAAGACCTTGTC | 10 |
| Human_respirovirus_3---NA.u1.g1 | CACAC (SEQ ID NO: 604) | |
| Paramyxoviridae---Respirovirus--- | ACCCTGAGATGCTAGTGA (SEQ ID NO: 605) | 10 |
| Human_respirovirus_3---NA.u1.g2 | | |
| Paramyxoviridae---Respirovirus--- | gtTAATACGACTCACTATAGGGGGAGGAGGTGCT | 10 |
| Murine_respirovirus---NA.u1.g1 | GTTATC (SEQ ID NO: 606) | |
| Paramyxoviridae---Respirovirus--- | CTAGGAAGGTGGTTGCAA (SEQ ID NO: 607) | 10 |
| Murine_respirovirus---NA.u1.g2 | | |
| Paramyxoviridae---Rubulavirus--- | gtTAATACGACTCACTATAGGGCAAGTTCACCTGC | 10 |
| Human_rubulavirus_2---NA.u1.g1 | ACATG (SEQ ID NO: 608) | |
| Paramyxoviridae---Rubulavirus--- | GTCTGAAGGCGAAGATC (SEQ ID NO: 609) | 10 |
| Human_rubulavirus_2---NA.u1.g2 | | |
| Paramyxoviridae---Rubulavirus--- | gtTAATACGACTCACTATAGGGCATGGGAGTTGG | 10 |
| Human_rubulavirus_4---NA.u1.g1 | AAGTGT (SEQ ID NO: 610) | |
| Paramyxoviridae---Rubulavirus--- | CCTGGTGTTTCATTGCAG (SEQ ID NO: 611) | 10 |
| Human_rubulavirus_4---NA.u1.g2 | | |
| Paramyxoviridae---Rubulavirus--- | gtTAATACGACTCACTATAGGGGGCCCAAGATGCT | 10 |
| Mammalian_rubulavirus_5---NA.u1.g1 | ATCAT (SEQ ID NO: 612) | |
| Paramyxoviridae---Rubulavirus--- | CTCCCCAGTAGGATCCTT (SEQ ID NO: 613) | 10 |
| Mammalian_rubulavirus_5---NA.u1.g2 | | |
| Parvoviridae---Erythroparvovirus--- | gtTAATACGACTCACTATAGGGAACTCAGTGGCA | 10 |
| Primate_erythroparvovirus_1---NA.u1.g1 | GCT (SEQ ID NO: 614) | |
| Parvoviridae---Erythroparvovirus--- | GCTACAACTTCGGAGGAA (SEQ ID NO: 615) | 10 |
| Primate_erythroparvovirus_1---NA.u1.g2 | | |
| Peribunyaviridae---Orthobunyavirus--- | gtTAATACGACTCACTATAGGGATAAGACGCCACA | 10 |
| Akabane_orthobunyavirus---S.u1.g1 | ACCAA (SEQ ID NO: 616) | |
| Peribunyaviridae---Orthobunyavirus--- | TGACACTGGATTTGCAGT (SEQ ID NO: 617) | 10 |
| Akabane_orthobunyavirus---S.u1.g2 | | |
| Peribunyaviridae---Orthobunyavirus--- | gtTAATACGACTCACTATAGGGTAAGCGTATCCAC | 10 |
| Bunyannwera_orthobunyavirus---S.u1.g1 | ACCAC (SEQ ID NO: 618) | |
| Peribunyaviridae---Orthobunyavirus--- | CCCCAAGGTTAAGCGTAA (SEQ ID NO: 619) | 10 |
| Bunyannwera_orthobunyavirus---S.u1.g2 | | |
| Peribunyaviridae---Orthobunyavirus--- | gtTAATACGACTCACTATAGGGAATTTGGAGAGT | 10 |
| California_encephalitis_orthobunyavirus---S.u1.g1 | GGCAGG (SEQ ID NO: 620) | |
| Peribunyaviridae---Orthobunyavirus--- | TGGATGGTAAGATCGTTGT (SEQ ID NO: 621) | 10 |
| California_encephalitis_orthobunyavirus---S.u1.g2 | | |
| Peribunyaviridae---Orthobunyavirus--- | gtTAATACGACTCACTATAGGGAGTCCAGTCCTCG | 10 |
| Guaroa_orthobunyavirus---S.u1.g1 | ATGAT (SEQ ID NO: 622) | |
| Peribunyaviridae---Orthobunyavirus--- | CTTGCTCAGGTGCTGATA (SEQ ID NO: 623) | 10 |
| Guaroa_orthobunyavirus---S.u1.g2 | | |
| Peribunyaviridae---Orthobunyavirus--- | gtTAATACGACTCACTATAGGGGATGTACCACAAC | 10 |
| Oropouche_orthobunyavirus---S.u1.g1 | GGACT (SEQ ID NO: 624) | |
| Peribunyaviridae---Orthobunyavirus--- | TGAGCACTTGTCCGTATC (SEQ ID NO: 625) | 10 |
| Oropouche_orthobunyavirus---S.u1.g2 | | |
| Peribunyaviridae---Orthobunyavirus--- | gtTAATACGACTCACTATAGGGGCTGATCTTCTCA | 10 |
| Sathuperi_orthobunyavirus---LuLg1 | TGGCT (SEQ ID NO: 626) | |
| Peribunyaviridae---Orthobunyavirus--- | GCGAATGTTGGCAGT (SEQ ID NO: 627) | 10 |
| Sathuperi_orthobunyavirus---LuLg2 | | |
| Peribunyaviridae---Orthobunyavirus--- | gtTAATACGACTCACTATAGGGICTCGCTACGTTT | 10 |
| Shuni_orthobunyavirus---S.u1.g1 | AACCC (SEQ ID NO: 628) | |
| Peribunyaviridae---Orthobunyavirus--- | GCCGTCTTACTGAGTACC (SEQ ID NO: 629) | 10 |
| Shuni_orthobunyavirus---S.u1.g2 | | |
| Phenuiviridae---Phlebovirus--- | gtTAATACGACTCACTATAGGGGGAGACAATAGC | 11 |
| Rift_Valley_fever_phlebovirus---L.u1.g1 | CAGGTC (SEQ ID NO: 630) | |
| Phenuiviridae---Phlebovirus--- | GATGTTGCACAAGTCCAC (SEQ ID NO: 631) | 11 |
| Rift_Valley_fever_phlebovirus---L.u1.g2 | | |
| Phenuiviridae---Phlebovirus--- | gtTAATACGACTCACTATAGGGTGAATCATGCAAG | 11 |
| Sandfly_fever_Naples_phlebovirus---M.u1.g1 | GGTGT (SEQ ID NO: 632) | |
| Phenuiviridae---Phlebovirus--- | GCACTATGCCTCCTTAGAA (SEQ ID NO: 633) | 11 |
| Sandfly_fever_Naples_phlebovirus---M.u1.g2 | | |
| Phenuiviridae---Phlebovirus--- | gtTAATACGACTCACTATAGGGTGAGTCATGCGGT | 11 |
| Sandfly_fever_Naples_phlebovirus---M.u1.g3 | GT (SEQ ID NO: 634) | |
| Phenuiviridae---Phlebovirus--- | GCACTATGCCTTCGTAGA (SEQ ID NO: 635) | 11 |
| Sandfly_fever_Naples_phlebovirus---M.u1.g4 | | |
| Phenuiviridae---Phlebovirus--- | gtTAATACGACTCACTATAGGGGGGTCCAGCTTGC | 11 |
| Sandfly_fever_Sicilian_virus---S.u1.g1 | TAC (SEQ ID NO: 636) | |
| Phenuiviridae---Phlebovirus--- | GTGAGCATCCAATACTGC (SEQ ID NO: 637) | 11 |
| Sandfly_fever_Sicilian_virus---S.u1.g2 | | |
| Phenuiviridae---Phlebovirus--- | gtTAATACGACTCACTATAGGGGGGAGCACAATG | 11 |
| Sandfly_fever_Sicilian_virus---S.u1.g3 | GACC (SEQ ID NO: 638) | |
| Phenuiviridae---Phlebovirus--- | GTGGCCAGCTGAGAG (SEQ ID NO: 639) | 11 |
| Sandfly_fever_Sicilian_virus---S.u1.g4 | | |
| Phenuiviridae---Phlebovirus--- | gtTAATACGACTCACTATAGGGGGCCCAGCATGCT | 11 |
| Sandfly_fever_Sicilian_virus---S.u1.g5 | AC (SEQ ID NO: 640) | |
| Phenuiviridae---Phlebovirus--- | GCCAACTGAGTGCCTTA (SEQ ID NO: 641) | 11 |
| Sandfly_fever_Sicilian_virus---S.u1.g6 | | |
| Phenuiviridae---Phlebovirus---SFTS_phlebovirus--- | gtTAATACGACTCACTATAGGGTCTACGACAGGCC | 11 |
| L.u1.g1 | AG (SEQ ID NO: 642) | |
| Phenuiviridae---Phlebovirus---SFTS_phlebovirus--- | TGTGATCAACCCAGCATT SEQ ID NO: 643) | 11 |
| L.u1.g2 | | |
| Phenuiviridae---Phlebovirus--- | gtTAATACGACTCACTATAGGGGATTTGATGCTAC | 11 |
| Uukunienni_phlebovirus---S.u1.g1 | TGTGGT (SEQ ID NO: 644) | |
| Phenuiviridae---Phlebovirus--- | TTCTCCTACCATCTGCTTG (SEQ ID NO: 645) | 11 |
| Uukunienni_phlebovirus---S.u1.g2 | | |
| Phenuiviridae---Phlebovirus--- | gtTAATACGACTCACTATAGGGTTTGATGCAGCCG | 11 |
| Uukunienni_phlebovirus---S.u1.g3 | TGG (SEQ ID NO: 646) | |
| Phenuiviridae---Phlebovirus--- | TGTCCCGGATCATCTGAT (SEQ ID NO: 647) | 11 |
| Uukunienni_phlebovirus---S.u1.g4 | | |
| Phenuiviridae---Phlebovirus--- | gtTAATACGACTCACTATAGGGTGTGGGCTTTTCT | 11 |
| Uukunienni_phlebovirus---S.u1.g5 | GTCAT (SEQ ID NO: 648) | |
| Phenuiviridae---Phlebovirus--- | TGTCCCTCATCATCTGGT (SEQ ID NO: 649) | 11 |
| Uukunienni_phlebovirus---S.u1.g6 | | |
| Picornaviridae---Aphthovirus---Foot-and- | gtTAATACGACTCACTATAGGGGGTGACAGGCTA | 11 |
| mouth_disease_virus---NA.u1.g1 | AGGATG (SEQ ID NO: 650) | |
| Picornaviridae---Aphthovirus---Foot-and- | CTCCGGTCACCTATTCAG (SEQ ID NO: 651) | 11 |
| mouth_disease_virus---NA.u1.g2 | | |
| Picornaviridae---Cardiovirus---Cardiovirus_A--- | gtTAATACGACTCACTATAGGGATTCAACAAGGG | 11 |
| NA.u1.g1 | GCTGAA (SEQ ID NO: 652) | |
| Picornaviridae---Cardiovirus---Cardiovirus_A--- | CGGACCACGTCC (SEQ ID NO: 653) | 11 |
| NA.u1.g2 | | |
| Picornaviridae---Cardiovirus---Cardiovirus_B--- | gtTAATACGACTCACTATAGGGATCATGCCTCCCC | 11 |
| NA.u1.g1 | GATTA (SEQ ID NO: 654) | |
| Picornaviridae---Cardiovirus---Cardiovirus_B--- | TCATATTCCAAGCGGCTT (SEQ ID NO: 655) | 11 |
| NA.u1.g2 | | |
| Picornaviridae---Enterovirus---Enterovirus_A--- | gtTAATACGACTCACTATAGGGATTCATGTCACCT | 12 |
| NA.u1.g1 | GCGAG (SEQ ID NO: 656) | |
| Picornaviridae---Enterovirus---Enterovirus_A--- | GTGCCCATCATGTTATT (SEQ ID NO: 657) | 12 |
| NA.u1.g10 | | |
| Picornaviridae---Enterovirus---Enterovirus_A--- | gtTAATACGACTCACTATAGGGCATGTCACCCGCG | 12 |
| NA.u1.g11 | AG (SEQ ID NO: 658) | |
| Picornaviridae---Enterovirus---Enterovirus_A--- | AGTGCCCATCATGTTGTT (SEQ ID NO: 659) | 12 |
| NA.u1.g2 | | |
| Picornaviridae---Enterovirus---Enterovirus_A--- | gtTAATACGACTCACTATAGGGCATTCATGTCACC | 12 |
| NA.u1.g3 | TGCTAG (SEQ ID NO: 660) | |
| Picornaviridae---Enterovirus---Enterovirus_A--- | ATGGCCCATCATGTTGTT (SEQ ID NO: 661) | 12 |
| NA.u1.g4 | | |
| Picornaviridae---Enterovirus---Enterovirus_A--- | gtTAATACGACTCACTATAGGGTTTCATGTCACCA | 12 |
| NA.u1.g5 | GCCAG (SEQ ID NO: 662) | |
| Picornaviridae---Enterovirus---Enterovirus_A--- | ACGTACCCATCATGTTGT (SEQ ID NO: 663) | 12 |
| NA.u1.g6 | | |
| Picornaviridae---Enterovirus---Enterovirus_A--- | gtTAATACGACTCACTATAGGGCCTTCATGTCACC | 12 |
| NA.u1.g7 | AGCTA (SEQ ID NO: 664) | |
| Picornaviridae---Enterovirus---Enterovirus_A--- | AGGTGCCCATCATATTGT (SEQ ID NO: 665) | 12 |
| NA.u1.g8 | | |
| Picornaviridae---Enterovirus---Enterovirus_A--- | gtTAATACGACTCACTATAGGGTCATGTCGCCAGC | 12 |
| NA.u1.g9 | AAC (SEQ ID NO: 666) | |
| Picornaviridae---Enterovirus---Enterovirus_B--- | gtTAATACGACTCACTATAGGGTGCGGCTAATCCT | 12 |
| NA.u1.g1 | AACTG (SEQ ID NO: 667) | |
| Picornaviridae---Enterovirus---Enterovirus_B--- | CACCCGTAGTCGGTT (SEQ ID NO: 668) | 12 |
| NA.u1.g2 | | |
| Picornaviridae---Enterovirus---Enterovirus_C--- | gtTAATACGACTCACTATAGGGGACTACTTTGGGT | 12 |
| NA.u1.g1 | GTCCG (SEQ ID NO: 669) | |
| Picornaviridae---Enterovirus---Enterovirus_C--- | GCCAATCCAATTCGCTTT (SEQ ID NO: 670) | 12 |
| NA.u1.g2 | | |
| Picornaviridae---Enterovirus---Enterovirus_D--- | gtTAATACGACTCACTATAGGGCTCAAGGTGTCCC | 12 |
| NA.u1.g1 | AACAT (SEQ ID NO: 671) | |
| Picornaviridae---Enterovirus---Enterovirus_D--- | GAGTTGGGTTGCACG (SEQ ID NO: 672) | 12 |
| NA.u1.g2 | | |
| Picornaviridae---Enterovirus---Enterovirus_E--- | gtTAATACGACTCACTATAGGGCTAATCCCAACCT | 12 |
| NA.u1.g1 | CCGAG (SEQ ID NO: 673) | |
| Picornaviridae---Enterovirus---Enterovirus_E--- | GTAGTCTGTTCCGCC (SEQ ID NO: 674) | 12 |
| NA.u1.g2 | | |
| Picornaviridae---Enterovirus---Rhinovirus_A--- | gtTAATACGACTCACTATAGGGCCCCTGAATGTGG | 12 |
| NA.u1.g1 | CTAAC (SEQ ID NO: 675) | |
| Picornaviridae---Enterovirus---Rhinovirus_A--- | CGGACACCCGTAGTT (SEQ ID NO: 676) | 12 |
| NA.u1.g2 | | |
| Picornaviridae---Enterovirus---Rhinovirus_B--- | gtTAATACGACTCACTATAGGGCTGAATGCGGCTA | 12 |
| NA.u1.g1 | ACCT (SEQ ID NO: 677) | |
| Picornaviridae---Enterovirus---Rhinovirus_B--- | CGTAGTCGGTCCCAT (SEQ ID NO: 678) | 12 |
| NA.u1.g2 | | |
| Picornaviridae---Enterovirus---Rhinovirus_C--- | gtTAATACGACTCACTATAGGGCCCTGAATGCGGC | 12 |
| NA.u1.g1 | TAAT (SEQ ID NO: 679) | |
| Picornaviridae---Enterovirus---Rhinovirus_C--- | CACCCGTAGTCGGTT (SEQ ID NO: 680) | 12 |
| NA.u1.g2 | | |
| Picornaviridae---Hepatovirus---Hepatovirus_A--- | gtTAATACGACTCACTATAGGGAGTCTTTGGGGAC | 12 |
| NA..u1.g1 | GC (SEQ ID NO: 681) | |
| Picornaviridae---Hepatovirus---Hepatovirus_A--- | CCTAAGAGGTTTCACCCG (SEQ ID NO: 682) | 12 |
| NA..u1.g2 | | |
| Picornaviridae---Kobuvirus---Aichivirus_A--- | gtTAATACGACTCACTATAGGGCACGATCTATGAA | 12 |
| NA.u1.g1 | GTCACC (SEQ ID NO: 683) | |
| Picornaviridae---Kobuvirus---Aichivirus_A--- | TCTCCATCACGCAAC (SEQ ID NO: 684) | 12 |
| NA.u1.g2 | | |
| Picornaviridae---Parechovirus---Parechovirus_A--- | gtTAATACGACTCACTATAGGGGCCAGCCAAGGTT | 12 |
| NA.u1.g1 | TA (SEQ ID NO: 685) | |
| Picornaviridae---Parechovirus---Parechovirus_A--- | TACCTTCTGGGCATCCTT (SEQ ID NO: 686) | 12 |
| NA.u1.g2 | | |
| Pneumoviridae---Metapneumovirus--- | gtTAATACGACTCACTATAGGGAGCTGCAATTAGT | 13 |
| Avian_metapneumovirus---NA.u1.g1 | GGGG (SEQ ID NO: 687) | |
| Pneumoviridae---Metapneumovirus--- | TTAGGGTTGTTCATTGTCAT (SEQ ID NO: 688) | 13 |
| Avian_metapneumovirus---NA.u1.g2 | | |
| Pneumoviridae---Metapneumovirus--- | gtTAATACGACTCACTATAGGGAGAGGCTGCAGA | 13 |
| Human_metapneumovirus---NA.u1.g1 | ACA (SEQ ID NO: 689) | |
| Pneumoviridae---Metapneumovirus--- | TTGCTGCTTCATTACCCA (SEQ ID NO: 690) | 13 |
| Human_metapneumovirus---NA.u1.g2 | | |
| Pneumoviridae---Orthopneumovirus--- | gtTAATACGACTCACTATAGGGTGGGGCTATGGC | 13 |
| Human_orthopneumovirus---NA.u1.g1 | (SEQ ID NO: 691) | |
| Pneumoviridae---Orthopneumovirus--- | GGCACCCATATTGTAAGTG (SEQ ID NO: 692) | 13 |
| Human_orthopneumovirus---NA.u1.g2 | | |
| Pneumoviridae------Respiratory_syncytial_virus--- | gtTAATACGACTCACTATAGGGGAGGTGGCTCCA | 13 |
| NA.u1.g1 | GAATAC (SEQ ID NO: 693) | |
| Pneumoviridae------Respiratory_syncytial_virus--- | TCTATCCCCTGCTGCTAA (SEQ ID NO: 694) | 13 |
| NA.u1.g2 | | |
| Polyomaviridae---Alphapolyomavirus--- | gtTAATACGACTCACTATAGGGTATTTGGTGCTTG | 13 |
| Human_polyomavirus_5---NA.u1.g1 | CCTGA (SEQ ID NO: 695) | |
| Polyomaviridae---Alphapolyomavirus--- | GTCCTGACCAGCTTCTAC (SEQ ID NO: 696) | 13 |
| Human_polyomavirus_5---NA.u1.g2 | | |
| Polyomaviridae---Betapolyomavirus--- | gtTAATACGACTCACTATAGGGCACAGGAGGGGA | 13 |
| Human_polyomavirus_1---NA.u1.g1 | TGT (SEQ ID NO: 697) | |
| Polyomaviridae---Betapolyomavirus--- | CTTTACGAGGCCCCA (SEQ ID NO: 698) | 13 |
| Human_polyomavirus_1---NA.u1.g2 | | |
| Polyomaviridae---Betapolyomavirus--- | gtTAATACGACTCACTATAGGGACAGAAGGACCC | 13 |
| Human_polyomavirus_2---NA.u1.g1 | CTAGAG (SEQ ID NO: 699) | |
| Polyomaviridae---Betapolyomavirus--- | CTCATCATGTCTGGGTCC (SEQ ID NO: 700) | 13 |
| Human_polyomavirus_2---NA.u1.g2 | | |
| Polyomaviridae---Betapolyomavirus--- | gtTAATACGACTCACTATAGGGGTGTAACACCCAC | 13 |
| Human_polyomavirus_3---NA.u1.g1 | AGACA (SEQ ID NO: 701) | |
| Polyomaviridae---Betapolyomavirus--- | GCAGTGTTCTAGGGTCTC (SEQ ID NO: 702) | 13 |
| Human_polyomavirus_3---NA.u1.g2 | | |
| Polyomaviridae---Betapolyomavirus--- | gtTAATACGACTCACTATAGGGAATTAGCAGCCAC | 13 |
| Human_polyomavirus_4---NA.u1.g1 | AAGGT (SEQ ID NO: 703) | |
| Polyomaviridae---Betapolyomavirus--- | TAGGTCACAGCTGCA (SEQ ID NO: 704) | 13 |
| Human_polyomavirus_4---NA.u1.g2 | | |
| Polyomaviridae---Betapolyomavirus--- | gtTAATACGACTCACTATAGGGTTGGGGTCCAACA | 13 |
| Macaca_mulatta_polyomavirus_1---NA.u1.g1 | CTTTT (SEQ ID NO: 705) | |
| Polyomaviridae---Betapolyomavirus--- | GGTGAGCCTAGGAATGTC (SEQ ID NO: 706) | 13 |
| Macaca_mulatta_polyomavirus_1---NA.u1.g2 | | |
| Poxviridae---Orthopoxvirus---Cowpox_virus--- | gtTAATACGACTCACTATAGGGCTACGGGCATTGT | 13 |
| NA.u1.g1 | CATCT (SEQ ID NO: 707) | |
| Poxviridae---Orthopoxvirus---Cowpox_virus--- | GCTCGCTTTACAGATCCT (SEQ ID NO: 708) | 13 |
| NA.u1.g2 | | |
| Poxviridae---Orthopoxvirus---Monkeypox_virus--- | gtTAATACGACTCACTATAGGGCACCGCAATAGAT | 13 |
| NA.u1.g1 | CCTGT (SEQ ID NO: 709) | |
| Poxviridae---Orthopoxvirus---Monkeypox_virus--- | AATATGTCCGCCGTTCAT (SEQ ID NO: 710) | 13 |
| NA.u1.g2 | | |
| Poxviridae---Orthopoxvirus---Vaccinia_virus--- | gtTAATACGACTCACTATAGGGACACGCTGGACA | 13 |
| NA.u1.g1 | ATCTAG (SEQ ID NO: 711) | |
| Poxviridae---Orthopoxvirus---Vaccinia_virus--- | GGTGGAGGTCTGAGAATG (SEQ ID NO: 712) | 13 |
| NA.u1.g2 | | |
| Poxviridae---Orthopoxvirus---Variola_virus--- | gtTAATACGACTCACTATAGGGGGACCCCAACATC | 13 |
| NA.u1.g1 | TTTGA (SEQ ID NO: 713) | |
| Poxviridae---Orthopoxvirus---Variola_virus--- | GACCTCACCGACGAT (SEQ ID NO: 714) | 13 |
| NA.u1.g2 | | |
| Poxviridae---Parapoxvirus---Orf_virus---NA.u1.g1 | gtTAATACGACTCACTATAGGGGGCAACCCCGATT | 13 |
| | ATGTA (SEQ ID NO: 715) | |
| Poxviridae---Parapoxvirus---Orf_virus---NA.u1.g2 | GTCAAGGACTGGATAGCC (SEQ ID NO: 716) | 13 |
| Reoviridae---Orbivirus---Greatisland_virus--- | gtTAATACGACTCACTATAGGGTCGGAGACCTCGA | 14 |
| 1.u1.g1 | AGC (SEQ ID NO: 717) | |
| Reoviridae---Orbivirus---Greatisland_virus--- | TGTGCGTGTCGTAATTTG (SEQ ID NO: 718) | 14 |
| 1.u1.g2 | | |
| Reoviridae---Orbivirus---Greatisland_virus--- | gtTAATACGACTCACTATAGGGTAATTGGCGACCT | 14 |
| 1.u1.g3 | GGAG (SEQ ID NO: 719) | |
| Reoviridae---Orbivirus---Greatisland_virus--- | ATGTGGGTGTCGTAGTTC (SEQ ID NO: 720) | 14 |
| 1.u1.g4 | | |
| Reoviridae---Orthoreovirus--- | gtTAATACGACTCACTATAGGGGGACCGCTGAAT | 14 |
| Mammalian_orthoreovirus---L1.u1.g1 | ACCTAA (SEQ ID NO: 721) | |
| Reoviridae---Orthoreovirus--- | AACAATTGGATGACGGCT (SEQ ID NO: 722) | 14 |
| Mammalian_orthoreovirus---L1.u1.g2 | | |
| Reoviridae---Orthoreovirus--- | gtTAATACGACTCACTATAGGGGGACTGCCGAAT | 14 |
| Mammalian_orthoreovirus---L1.u1.g3 | ACCTAA (SEQ ID NO: 723) | |
| Reoviridae---Orthoreovirus--- | CACGATTGGATGACGACT (SEQ ID NO: 724) | 14 |
| Mammalian_orthoreovirus---L1.u1.g4 | | |
| Reoviridae---Rotavirus---Rotavirus_A---11.u1.g1 | gtTAATACGACTCACTATAGGGtGGACCATCTGAT | 14 |
| | TCTGC (SEQ ID NO: 725) | |
| Reoviridae---Rotavirus---Rotavirus_A---11.u1.g2 | AATCCATAGACACGCCAG (SEQ ID NO: 726) | 14 |
| Reoviridae---Rotavirus---Rotavirus_B---4.u1.g1 | gtTAATACGACTCACTATAGGGTATCGTGICCTIG | 14 |
| | AGCAC (SEQ ID NO: 727) | |
| Reoviridae---Rotavirus---Rotavirus_B---4.u1.g2 | GTCCCCTGTACACCA (SEQ ID NO: 728) | 14 |
| Reoviridae---Rotavirus---Rotavirus_C---2.u1.g1 | gtTAATACGACTCACTATAGGGCGCACGCTGATTA | 14 |
| | TGTTT (SEQ ID NO: 729) | |
| Reoviridae---Rotavirus---Rotavirus_C---2.u1.g2 | TGTGCAGCCATTTCTTTT (SEQ ID NO: 730) | 14 |
| Reoviridae---Rotavirus---Rotavirus_C---2.u1.g3 | gtTAATACGACTCACTATAGGGCGCATGCGGATTA | 14 |
| | TGTATC (SEQ ID NO: 731) | |
| Reoviridae---Rotavirus---Rotavirus_C---2.u1.g4 | GTGCTGCCATTTCTTTCA (SEQ ID NO: 732) | 14 |
| Reoviridae---Rotavirus---Rotavirus_C---2.u1.g5 | gtTAATACGACTCACTATAGGGCACATGCTGATTA | 14 |
| | CGTTTC (SEQ ID NO: 733) | |
| Reoviridae---Rotavirus---Rotavirus_C---2.u1.g6 | GCCGCCATTTCTTTCAT (SEQ ID NO: 734) | 14 |
| Reoviridae---Rotavirus---Rotavirus_H---6.u1.g1 | gtTAATACGACTCACTATAGGGATCTACTTGCACC | 14 |
| | AGGTG (SEQ ID NO: 735) | |
| Reoviridae---Rotavirus---Rotavirus_H---6.u1.g2 | GGTACTTTCATGTCAAGTGC (SEQ ID NO: 736) | 14 |
| Reoviridae---Seadornavirus---Banna_virus--- | gtTAATACGACTCACTATAGGGTTGATTTCCAGCA | 14 |
| 12.u1.g1 | CCAGT (SEQ ID NO: 737) | |
| Reoviridae---Seadornavirus---Banna_virus--- | ACTCTGGCTTGAATGTTTT (SEQ ID NO: 738) | 14 |
| 12. u 1.g2 | | |
| Retroviridae---Deltaretrovirus---Primate_T- | gtTAATACGACTCACTATAGGGGCTAATACGCCTC | 14 |
| lymphotropic_virus_1---NA.u1.g1 | CCTTT (SEQ ID NO: 739) | |
| Retroviridae---Deltaretrovirus---Primate_T- | AAGGCATCACGACCTATG (SEQ ID NO: 740) | 14 |
| lymphotropic_virus_1---NA.u1.g2 | | |
| Retroviridae--- Delta retrovi rus---Primate_T- | gtTAATACGACTCACTATAGGGTAGACCTTACTGA | 14 |
| lymphotropic_virus_2---NA.u1.g1 | CGCCT (SEQ ID NO: 741) | |
| Retroviridae---Deltaretrovirus---Primate_T- | CCGGGGCCATAATTACAT (SEQ ID NO: 742) | 14 |
| lymphotropic_virus_2---NA.u1.g2 | | |
| Retroviridae---Lentivirus--- | gtTAATACGACTCACTATAGGGGAGAGGCTGGCA | 14 |
| Human_immunodeficiency_virus_2---NA.u1.g1 | GATTG (SEQ ID NO: 743) | |
| Retroviridae---Lentivirus--- | AGAGTCTAGCAGGGAACA (SEQ ID NO: 744) | 14 |
| Human_immunodeficiency_virus_2---NA.u1.g2 | | |
| Rhabdoviridae---Lyssavirus--- | gtTAATACGACTCACTATAGGGCAGGATTAGACTG | 15 |
| European_bat_1_lyssavirus---NA.u1.g1 | GGCTG (SEQ ID NO: 745) | |
| Rhabdoviridae---Lyssavirus--- | GGCTATCTGATGGGCAAT (SEQ ID NO: 746) | 15 |
| European_bat_1_lyssavirus---NA.u1.g2 | | |
| Rhabdoviridae---Lyssavirus--- | gtTAATACGACTCACTATAGGGCAGACGATGAGG | 15 |
| European_bat_2_lyssavirus---NA.u1.g1 | AGCTTT (SEQ ID NO: 747) | |
| Rhabdoviridae---Lyssavirus--- | CTTTCCCCCATTGACCAT (SEQ ID NO: 748) | 15 |
| European_bat_2_lyssavirus---NA.u1.g2 | | |
| Rhabdoviridae---Vesiculovirus--- | gtTAATACGACTCACTATAGGGAACGAGCTGAGT | 15 |
| Indiana_vesiculovirus---NA.u1.g1 | CCA (SEQ ID NO: 749) | |
| Rhabdoviridae---Vesiculovirus--- | TCATCTGCTGCCTGA (SEQ ID NO: 750) | 15 |
| Indiana_vesiculovirus---NA.u1.g2 | | |
| Rhabdoviridae---Vesiculovirus--- | gtTAATACGACTCACTATAGGGATTTGGCCTAGAG | 15 |
| New_Jersey_vesiculovirus---NA.u1.g1 | GGAAC (SEQ ID NO: 751) | |
| Rhabdoviridae---Vesiculovirus---New_Jersey_ | TTGAAGTAATCAGCCGGG (SEQ ID NO: 752) | 15 |
| vesiculovirus---NA.u1.g2 | | |
| Smacoviridae------Human_smacovirus_1---NA.u1.g1 | gtTAATACGACTCACTATAGGGCTTAACCTGTCCT | 15 |
| | CCGAC (SEQ ID NO: 753) | |
| Smacoviridae------Human_smacovirus_1---NA.u1.g2 | AATGGGTACATGTGGGAC (SEQ ID NO: 754) | 15 |
| Smacoviridae------Human_smacovirus_1---NA.u1.g3 | gtTAATACGACTCACTATAGGGCCTGAACCGGTCT | 15 |
| | TCTG (SEQ ID NO: 755) | |
| Smacoviridae------Human_smacovirus_1---NA.u1.g4 | ACGGTTACTTATGGGACG (SEQ ID NO: 756) | 15 |
| Togaviridae---Alphavirus--- | gtTAATACGACTCACTATAGGGGCAGTGGACCATT | 15 |
| Eastern_equine_encephalitis_virus---NA.u1.g1 | TGAAC (SEQ ID NO: 757) | |
| Togaviridae---Alphavirus--- | TAATGTTCTCGGTGGCTC (SEQ ID NO: 758) | 15 |
| Eastern_equine_encephalitis_virus---NA.u1.g2 | | |
| Togaviridae---Alphavirus---Getah_virus---NA.u1.g1 | gtTAATACGACTCACTATAGGGTACGCAGTTACCC | 15 |
| | ATCAC (SEQ ID NO: 759) | |
| Togaviridae---Alphavirus---Getah_virus---NA.u1.g2 | GTACAGACCGGGGAG (SEQ ID NO: 760) | 15 |
| Togaviridae---Alphavirus---Highlands_Lvirus--- | gtTAATACGACTCACTATAGGGCCTGGACAGCGG | 15 |
| NA.u1.g1 | ATTATT (SEQ ID NO: 761) | |
| Togaviridae---Alphavirus---Highlands_Lvirus--- | GGCGAATTATCCCAGTGA (SEQ ID NO: 762) | 15 |
| NA.u1.g2 | | |
| Togaviridae---Alphavirus---Mayaro_virus---NA.u1.g1 | gtTAATACGACTCACTATAGGGAGAGGTGGCAGT | 15 |
| | CTATCA (SEQ ID NO: 763) | |
| Togaviridae---Alphavirus---Mayaro_virus---NA.u1.g2 | GCGTACTCCTTTCATTGC (SEQ ID NO: 764) | 15 |
| Togaviridae---Alphavirus---Ross_River_virus--- | gtTAATACGACTCACTATAGGGTCCGTGTCTGTGT | 15 |
| NA.u1.g1 | AGGTA (SEQ ID NO: 765) | |
| Togaviridae---Alphavirus---Ross_River_virus--- | GACGCCTTCAATCCTGTA (SEQ ID NO: 766) | 15 |
| NA.u1.g2 | | |
| Togaviridae---Alphavirus---Semliki_Forest_virus--- | gtTAATACGACTCACTATAGGGGGACGTGTATGCT | 15 |
| NA.u1.g1 | GTACA (SEQ ID NO: 767) | |
| Togaviridae---Alphavirus---Semliki_Forest_virus--- | CAATCCAATACGCCGTTC (SEQ ID NO: 768) | 15 |
| NA.u1.g2 | | |
| Togaviridae---Alphavirus---Sindbis_virus---NA.u1.g1 | gtTAATACGACTCACTATAGGGATACTGACTAACC | 15 |
| | GGGGT (SEQ ID NO: 769) | |
| | TGCAGAACGGACTTCTTT (SEQ ID NO: 770) | 15 |
| Togaviridae---Alphavirus---Sindbis_virus---NA.u1.g2 | | |
| Togaviridae---Alphavirus--- | gtTAATACGACTCACTATAGGGTTGAGGTAGAAG | 15 |
| Venezuelan_equine_encephalitis_virus---NA.u1.g1 | CCAAGC (SEQ ID NO: 771) | |
| Togaviridae---Alphavirus--- | CGCACTTCCAATGTCAAG (SEQ ID NO: 772) | 15 |
| Venezuelan_equine_encephalitis_virus---NA.u1.g2 | | |
| Togaviridae---Alphavirus--- | gtTAATACGACTCACTATAGGGGCGATCGAGTGA | 15 |
| Western_equine_encephalitis_virus---NA.u1.g1 | TGC (SEQ ID NO: 773) | |
| Togaviridae---Alphavirus--- | GGTGAATGGCCTCGATTA (SEQ ID NO: 774) | 15 |
| Western_equine_encephalitis_virus---NA.u1.g2 | | |
| Togaviridae---Rubivirus---Rubella_virus---NA.u1.g1 | gtTAATACGACTCACTATAGGGGCAATTTCGCGGT | 15 |
| | ATACC (SEQ ID NO: 775) | |
| Togaviridae---Rubivirus---Rubella_virus---NA.u1.g2 | GTCGATGAGGACGTGTAG (SEQ ID NO: 776) | 15 |

TABLE 5a
HAV Round 2 Primers
Primers

| Name | Sequence | Pool |
|---|---|---|
| Orthohepevirus_kv2_fwd- | gaaatTAATACGACTCACTATAGGGAGGCCCACCAGTTCAT | 8v2 |
| 1 | (SEQ ID NO: 777) | |
| Orthohepevirus_kv2_fwd- | gaaatTAATACGACTCACTATAGGGGGAGGCCCATCAGTTTAT | 8v2 |
| 2 | (SEQ ID NO: 778) | |
| Orthohepevirus_A_v2_rev-1 | TACCACAGCATTCGCC (SEQ ID NO: 779) | 8v2 |
| Orthohepevirus_A_v2_rev-2 | ACAGCATTCGCCAAGG (SEQ ID NO: 780) | 8v2 |
| Rhinovirus_A_v2_fwd-1 | gaaatTAATACGACTCACTATAGGGGACAGGGTGTGAAGAGC | 12v2 |
| | (SEQ ID NO: 781) | |
| Rhinovirus_A_v2_fwd-2 | gaaatTAATACGACTCACTATAGGGTGACAAGGTGTGAAGAGC | 12v2 |
| | (SEQ ID NO: 782) | |
| Rhinovirus_A_v2_rev-1 | AAGTAGTTGGTCCCATCC (SEQ ID NO: 783) | 12v2 |
| Rhinovirus_A_v2_rev-2 | AAGTAGTCGGTCCCATCC (SEQ ID NO: 784) | 12v2 |
| Rhinovirus_B_v2_fwd-1 | gaaatTAATACGACTCACTATAGGGTAGTTTGGTCGATGAGGC | 12v2 |
| | (SEQ ID NO: 785) | |
| Rhinovirus_B_v2_rev-1 | CGGAGGACTCACAGTTAA (SEQ ID NO: 786) | 12v2 |
| Rhinovirus_B_v2_rev-2 | GGAGGACTCACAACCAAG (SEQ ID NO: 787) | 12v2 |

TABLE 5b
HAV Round 2 Targets and crRNAs
Targets

| Name | Sequence |
|---|---|
| Orthohepevirus_A_v2 | TGGAGGCCCATCAGTTTATTAAGGCTCCTGGCATCACTACTGCCATTGAGCAGGC |
| | TGCTCTGGCAGCGGCCAACTCCGCCTTGGCGAATGCTGTGGTG (SEQ ID |
| | NO: 788) |
| Rhinovirus_A_v2 | GGACAAGGTGTGAAGAGCCCCGTGTGCTCACTTTGAGTCCTCCGGCCCCTGAAT |
| | GTGGCTAACCTTAACCCTGCAGCCAGTGCACACAATCCAGTGTGTATCTGGTCGT |
| | AATGAGCAATTGCGGGATGGGACCAACTACTT (SEQ ID NO: 789) |
| Rhinovirus_B_v2 | CTAGTTTGGTCGATGAGGCTAGGAATTCCCCACGGGTGACCGTGTCCTAGCCTGC |
| | GTGGCGGCCAACCCAGCTTATGCTGGGACGCCTTTTTATAGACATGGTGTGAAG |
| | ACCCGCATGTGCTTGGTTGTGAGTCCTCCGG (SEQ ID NO: 790) |

crRNAs

| Name | Spacer sequence (RNA) |
|---|---|
| Orthohepevirus_A_v2a | Cggaguuggccgcugcuagagcugccug (SEQ ID NO: 791) |
| Rhinovirus_A_v2 | Gguuagccacauucaggggccggaggac (SEQ ID NO: 792) |
| Rhinovirus_B_v2 | Uuggccgccacgcaggcuaggacacggu (SEQ ID NO: 793) |
| Culex_flavivirus_v2 | Cagauugaacgccaacaucacguacauc (SEQ ID NO: 794) |
| Tula_v2 | Auuuuuugacuugauaccaaaucugcaa (SEQ ID NO: 795) |
| Betapap_1_v2a | Agcucuaauugauuccaaagccuuuuaa (SEQ ID NO: 796) |
| Getah_virus_v2b | Gacuguaucagugaucuuacacaucagg (SEQ ID NO: 797) |
| Zika_pilot_correct | Ccuuccagccguggggcagcucguucac (SEQ ID NO: 798) |
| Cowpox_v1_correct | Cgauuauaacaacagauauuauaauccu (SEQ ID NO: 799) |
| Kyasanur_forest_v2 | Auacccagccuuccacacgugucagaug (SEQ ID NO: 800) |
| Hepatitis_C_v2 | Acuccaccaacgaucugaccgccacccg (SEQ ID NO: 801) |

Diverse primer pool: 164 of the 169 hav10 species have designs with 3 or fewer primer pairs (total of 187 primer sequences required to cover them: 145 have 1 primer pair, 15 have 2 primer pairs, and 4 have 3 primer pairs). There were four species that required more than three primer pairs: Lymphocytic Choriomeningitis Virus (LCMV, 7 primer pairs), Norovirus (4 primer pairs), Betapapillomavirus 2 (6 primer pairs), and Candiru Phlebovirus (6 primer pairs). These four species were combined into a single “diverse” primer pool at 150 nM final concentration.

Degenerate primer pool: For 167 of the 169 hav10 species, it was possible to design primer sets using CATCH-dx/primer3 that cover >90% of the genomes in the database with fewer than 10 primer pairs. However, for two species (Simian Immunodeficiency Virus and Sapporo virus) it was not possible to identify sufficiently conserved pairs of primer binding sites using the computational design strategy. Instead, primers were designed with several degenerate bases to capture the extensive sequence diversity, and manually identified amplicons. These primers were used in a “degenerate” primer pool at 600 nM final concentration.

Remaining primer pools: For the remaining 149 hav10 species, Applicants pooled primers phylogenetically, such that each pool contained species from 1-3 viral genuses (see Table 4 for details). The primers for one species in pool 4 (Torque teno Leptonychotes weddellii virus-1) contain some degenerate bases, and were designed manually. These primers were used at 150 nM final concentration.

Version two redesign: After testing the hav10-v1 design, 3 amplicons were redesigned: Orthohepesvirus A, Rhinovirus A, and Rhinovirus B. The newly designed primers were re-pooled to create pools 8v2 and 12v2, and new crRNA sequences were designed to target these amplicons. Based on the results of the hav10-v1 testing Applicants redesigned crRNAs within the existing v1 amplicons for 14 species (see Table 5b).

A single replicate of the equivalent experiment conducted in 96 W plates would require ˜300 plates and >1 L of detection mix.

### Influenza A Design

Primer design: N primers were based on the majority consensus sequence for each subtype (9 primer pairs) in a single pool. CATCH-dx was used to design H primers covering at least 9500 of the sequences within each subtype. In total, there were 45 primers (15 forward primers, 30 reverse primers) in a single pool.

TABLE 6
Influenza Primers

| Primer name | Primer sequence | Notes |
|---|---|---|
| H_f1 | gaaatTAATACGACTCACTATAgggTGGACATACAATGCAGAATT | H amplification |
| | (SEQ ID NO: 802) | |
| H_f2 | gaaatTAATACGACTCACTATAgggTGGACATACAATGCTGAACT | H amplification |
| | (SEQ ID NO: 803) | |
| H_f3 | gaaatTAATACGACTCACTATAgggTGGACTTACAATGCTGAACT | H amplification |
| | (SEQ ID NO: 804) | |
| H_f4 | gaaatTAATACGACTCACTATAgggTGGACTTATCAGGCTGAACT | H amplification |
| | (SEQ ID NO: 805) | |
| H_f5 | gaaatTAATACGACTCACTATAgggTGGGCATATAATGCAGAATT | H amplification |
| | (SEQ ID NO: 806) | |
| H_f6 | gaaatTAATACGACTCACTATAgggTGGGCCTACAATGCAGAGCT | H amplification |
| | (SEQ ID NO: 807) | |
| H_f7 | gaaatTAATACGACTCACTATAgggTGGGCTTACAACGCAGAACT | H amplification |
| | (SEQ ID NO: 808) | |
| H_f8 | gaaatTAATACGACTCACTATAgggTGGTCATACAACGCACAGCT | H amplification |
| | (SEQ ID NO: 809) | |
| H_f9 | gaaatTAATACGACTCACTATAgggTGGTCATACAACGCGGAGCT | H amplification |
| | (SEQ ID NO: 810) | |
| H_f10 | gaaatTAATACGACTCACTATAgggTGGTCATACAATGCAAAACT | H amplification |
| | (SEQ ID NO: 811) | |
| H_f11 | gaaatTAATACGACTCACTATAgggTGGTCATACAATGCCGAATT | H amplification |
| | (SEQ ID NO: 812) | |
| H_f12 | gaaatTAATACGACTCACTATAgggTGGTCATATAATGCACAACT | H amplification |
| | (SEQ ID NO: 813) | |
| H_f13 | gaaatTAATACGACTCACTATAgggTGGTCATATAATGCAGAGCT | H amplification |
| | (SEQ ID NO: 814) | |
| H_f14 | gaaatTAATACGACTCACTATAgggTGGTCTTACAATGCTGAATT | H amplification |
| | (SEQ ID NO: 815) | |
| H_f15 | gaaatTAATACGACTCACTATAgggTGGACGTATCAAGCTGAATT | H amplification |
| | (SEQ ID NO: 816) | |
| H_r1 | AAAGCAGCCGTTTCCTATTT (SEQ ID NO: 817) | H amplification |
| H_r2 | AAAGCACCCGTTCCCTATTT (SEQ ID NO: 818) | H amplification |
| H_r3 | AAAGCACCCATTCCCTATTT (SEQ ID NO: 819) | H amplification |
| H_r4 | AAAGCAGCCATTTCCAATTT (SEQ ID NO: 820) | H amplification |
| H_r5 | AAAGCACCCATTTCCTAGTT (SEQ ID NO: 821) | H amplification |
| H_r6 | AAAACATCCATTCCCTAGTT (SEQ ID NO: 822) | H amplification |
| H_r7 | GAAACATCCTTTCCCTTCTT (SEQ ID NO: 823) | H amplification |
| H_r8 | GAAACATCCATTCCCTTCTT (SEQ ID NO: 824) | H amplification |
| H_r9 | AAAGCATCCAGTGCCATCTT (SEQ ID NO: 825) | H amplification |
| H_r10 | AAAACATCCTTTCCCATCTT (SEQ ID NO: 826) | H amplification |
| H_r11 | AAAGCACCCTTTCCCATCTT (SEQ ID NO: 827) | H amplification |
| H_r12 | AAAGCATCCGTTGCCCAATT (SEQ ID NO: 828) | H amplification |
| H_r13 | AAAACACCCGTTTCCTTTGT (SEQ ID NO: 829) | H amplification |
| H_r14 | AAAACATCCATTTCCTTTGT (SEQ ID NO: 830) | H amplification |
| H_r15 | AAAGCACCCATTTCCTTTGT (SEQ ID NO: 831) | H amplification |
| H_r16 | GAAACATCCATTCCCTTTGT (SEQ ID NO: 832) | H amplification |
| H_r17 | AAAGCACCCGTTCCCTAGGT (SEQ ID NO: 833) | H amplification |
| H_r18 | GAAGCAACCATTTCCTTCGT (SEQ ID NO: 834) | H amplification |
| H_r19 | GAAACAACCGTTACCCAGCT (SEQ ID NO: 835) | H amplification |
| H_r20 | AAAACATCCAGTCCCATCCT (SEQ ID NO: 836) | H amplification |
| H_r21 | AAAGCAACCATCTCCTGTAT (SEQ ID NO: 837) | H amplification |
| H_r22 | GAAGCAGCCATTCCCAGTAT (SEQ ID NO: 838) | H amplification |
| H_r23 | GAAACAACCATTGCCCATAT (SEQ ID NO: 839) | H amplification |
| H_r24 | GAAACAGCCGTTGCCTTGAT (SEQ ID NO: 840) | H amplification |
| H_r25 | AAAGCATCCGTTCCCTTCAT (SEQ ID NO: 841) | H amplification |
| H_r26 | GAAACATCCGTTCCCTTCAT (SEQ ID NO: 842) | H amplification |
| H_r27 | AAAACAACCATTCCCTTCAT (SEQ ID NO: 843) | H amplification |
| H_r28 | AAAACATCCATTCCCCTCAT (SEQ ID NO: 844) | H amplification |
| H_r29 | GAAGCAACCGTTCCCAGCAT (SEQ ID NO: 845) | H amplification |
| H_r30 | AAAGCAACCATTCCCAGCAT (SEQ ID NO: 846) | H amplification |
| N1-1087F | gaaatTAATACGACTCACTATAgggATGAGGAATGCTCMTGTTAY | N amplification |
| | (SEQ ID NO: 847) | |
| N2-1087F | gaaatTAATACGACTCACTATAgggTHGARGARTGCTCYTGYTAT | N amplification |
| | (SEQ ID NO: 848) | |
| N3-1087F | gaaatTAATACGACTCACTATAgggTRGARGARTGTTCHTGYTAY | N amplification |
| | (SEQ ID NO: 849) | |
| N4-1087F | gaaatTAATACGACTCACTATAgggTYGARGARTGTTCCTGTTAC | N amplification |
| | (SEQ ID NO: 850) | |
| N5-1087F | gaaatTAATACGACTCACTATAgggTWGARGARTGYTCYTGYTAY | N amplification |
| | (SEQ ID NO: 851) | |
| N6-1087F | gaaatTAATACGACTCACTATAgggTHGAAGARTGYTCRTGYTAY | N amplification |
| | (SEQ ID NO: 852) | |
| N7-1087F | gaaatTAATACGACTCACTATAgggTWGAGGARTGCTCMTGYTAY | N amplification |
| | (SEQ ID NO: 853) | |
| N8-1087F | gaaatTAATACGACTCACTATAgggTWGARGARTGYTCWTGYTAY | N amplification |
| | (SEQ ID NO: 854) | |
| N9-1087F | gaaatTAATACGACTCACTATAgggTTGAAGAATGCTCATGYTAY | N amplification |
| | (SEQ ID NO: 855) | |
| N1-1153R | SCATGCCARTTRTCYCTGCA (SEQ ID NO: 856) | N amplification |
| N2-1153R | CCYTTCCARTTGTCTCTGCA (SEQ ID NO: 857) | N amplification |
| N3-1153R | CCYTTCCARTTGTCYCTRCA (SEQ ID NO: 858) | N amplification |
| N4-1153R | CCYCKCCARTTGTCYCKACA (SEQ ID NO: 859) | N amplification |
| N5-1153R | CCRTTCCAATTRTCYCKGCA (SEQ ID NO: 860) | N amplification |
| N6-1153R | CCYTTCCAATTGTCYCTRCA (SEQ ID NO: 861) | N amplification |
| N7-1153R | CCYTGCCARTTRTCYCTGCA (SEQ ID NO: 862) | N amplification |
| N8-1153R | CCNGTCCARTTGTCYCTACA (SEQ ID NO: 863) | N amplification |
| N9-1153R | CCCTGCCAATTRTCYCTGCA (SEQ ID NO: 864) | N amplification |

crRNA design: sets consisting of a small number of crRNA sequences were designed to selectively target individual H or N subtypes using CATCH-dx. The design approach was improved throughout the process by incorporating new features into each round of design (FIG. 32). In the first round of design, Applicants only designed H crRNAs, and required that all crRNAs could hybridize 90% of all sequences, allowing for up to 1 mismatch. crRNAs in a set could be positioned anywhere in amplicon. In the second round of design, Applicants designed crRNAs for both H and N and restricted the positions of crRNAs within a set (to within a 91 nt window for H, and 35 nt window for N) as based on the sequence alignments, some positions within the amplicon were more conserved between subtypes than others. In addition, the coverage of the designs was weighted towards more recent years by introducing an exponential decay parameter for sequences older than 2017. In the third round, a differential design approach was implemented in which all crRNAs were required to have at least 3 mismatches when hybridizing to at least 99% of sequences within any other subtype. In the fourth round, the hybridization model was revised to account for G-U pairing, raising the threshold to 95% of sequences in each subtype, allowing for up to 1 mismatch. Each round of designs was tested experimentally, and high-performing crRNAs between designs were used in combination. H required 4 rounds of design, while N only required 2 (rounds 2 and 3).

TABLE 7
Influenza Targets

| Name | Sequence | Notes |
|---|---|---|
| 2k8_H1_majority- | TGGACTTACAATGCCGAACTGTTGGTTCTATTGGAAAATGAAAGAACTT | H |
| consensus | TGGACTACCACGATTCAAATGTGAAGAACTTATATGAAAAGGTAAGAA | subtyping |
| | GCCAGTTAAAAAACAATGCCAAGGAAATTGGAAACGGCTGCTTT (SEQ | |
| | ID NO: 865) | |
| 2k8_H2_majority- | TGGACATACAATGCCGAACTCCTAGTTCTAATGGAAAATGAGAGGACA | H |
| consensus | CTTGATTTCCATGACTCTAATGTAAGGAATCTGTACGATAAGGTCAGAA | subtyping |
| | TGCAACTGAGGGACAATGCTAAGGAAATAGGGAACGGATGCTTT (SEQ | |
| | ID NO: 866) | |
| 2k8_H3_majority- | TGGTCATACAACGCGGAGCTTCTTGTTGCCCTGGAGAACCAACATACAA | H |
| consensus | TTGATCTAACTGACTCAGAAATGAACAAACTGTTTGAAAAAACAAAGA | subtyping |
| | AGCAACTGAGGGAAAATGCTGAGGATATGGGCAATGGTTGTTTC (SEQ | |
| | ID NO: 867) | |
| 2k8_H4_majority- | TGGTCTTACAATGCTGAATTGCTGGTGGCATTAGAAAATCAACATACTA | H |
| consensus | TAGATGTGACAGACTCTGAAATGAACAAACTCTTTGAAAGAGTTAGGC | subtyping |
| | GCCAACTAAGAGAGAATGCTGAGGACAAAGGAAATGGATGTTTT (SEQ | |
| | ID NO: 868) | |
| 2k8_H5_majority- | TGGACTTATAATGCTGAACTTCTGGTTCTCATGGAAAATGAGAGAACTC | H |
| consensus | TAGACTTCCATGACTCAAATGTCAAGAACCTTTACGACAAGGTCCGACT | subtyping |
| | ACAGCTTAGGGATAATGCAAAGGAGCTGGGTAACGGTTGTTTC (SEQ | |
| | ID NO: 869) | |
| 2k8_H6_majority- | TGGACATACAATGCTGAACTGCTGGTTCTTCTTGAAAACGAAAGAACAC | H |
| consensus | TAGACCTGCATGATGCGAATGTGAAGAACCTATATGAAAAGGTCAAAT | subtyping |
| | CACAATTAAGGGACAATGCTAATGATCTAGGAAATGGGTGCTTT (SEQ | |
| | ID NO: 870) | |
| 2k8_H7_majority- | TGGTCATACAATGCTGAACTCTTGGTAGCAATGGAGAACCAGCATACA | H |
| consensus | ATTGATCTGGCTGATTCAGAAATGAACAAACTGTACGAACGAGTGAAA | subtyping |
| | AGACAGCTGAGAGAGAATGCTGAAGAAGATGGCACTGGTTGCTTT | |
| | (SEQ ID NO: 871) | |
| 2k8_H8_majority- | TGGGCTTACAATGCAGAACTCCTTGTACTTCTAGAAAACCAGAAAACAC | H |
| consensus | TAGACGAACATGACTCCAATGTCAAGAACCTCTTTGATGAAGTGAAAA | subtyping |
| | GGAGGTTGTCAACCAATGCAATAGATGCTGGGAACGGTTGCTTC (SEQ | |
| | ID NO: 872) | |
| 2k8_H9_majority- | TGGGCATATAATGCAGAATTGCTAGTTCTGCTTGAAAACCAGAAAACAC | H |
| consensus | TCGATGAGCATGACGCAAATGTAAACAATCTATATAATAAAGTGAAGA | subtyping |
| | GGGCGTTGGGTTCCAATGCGGTGGAAGATGGGAAAGGATGTTTC (SEQ | |
| | ID NO: 873) | |
| 2k8_H10_majority- | TGGACGTATCAAGCTGAATTGCTGGTAGCAATGGAAAATCAGCATACA | H |
| consensus | ATTGACATGGCTGATTCAGAAATGCTGAATCTATATGAGAGGGTGAGG | subtyping |
| | AAGCAACTAAGGCAAAATGCAGAAGAAGATGGGAAAGGGTGCTTT | |
| | (SEQ ID NO:874) | |
| 2k8_H11_majority- | TGGTCATACAACGCACAGCTTCTTGTTCTACTGGAAAATGAAAAAACAT | H |
| consensus | TAGATCTCCATGATTCTAATGTTCGAAACCTCCATGAAAAGGTCAGACG | subtyping |
| | AATGCTGAAGGACAATGCTAAAGATGAAGGGAATGGTTGTTTT (SEQ | |
| | ID NO: 875) | |
| 2k8_H12_majority- | TGGGCATACAATGCTGAACTGCTTGTTCTATTGGAAAATCAGAAGACAT | H |
| consensus | TAGATGAGCATGATGCTAATGTAAGGAATCTACATGATAGAGTCAGAA | subtyping |
| | GAGTCCTAAGGGAAAATGCAATTGATACAGGAGATGGTTGCTTT (SEQ | |
| | ID NO: 876) | |
| 2k8_H13_majority- | TGGTCATACAATGCAAAGCTTCTTGTTTTACTAGAAAACGACAAGACTC | H |
| consensus | TAGACATGCACGACGCTAATGTCAGGAACCTGCATGATCAAGTCCGCA | subtyping |
| | GAGTGCTGAGGACCAATGCAATTGATGAGGGGAATGGATGTTTT (SEQ | |
| | ID NO: 877) | |
| 2k8_H14_majority- | TGGTCATACAATGCTGAACTATTGGTGGCCCTGGAAAATCAGCACACA | H |
| consensus | ATAGATGTTACAGACTCCGAGATGAACAAACTCTTTGAAAGGGTGAGA | subtyping |
| | AGACAACTTAGGGAAAATGCGGAAGATCAAGGCAACGGCTGTTTC | |
| | (SEQ ID NO: 878) | |
| 2k8_H15_majority- | TGGTCATACAATGCCGAATTACTGGTGGCAATGGAAAATCAACACACA | H |
| consensus | ATTGACCTTGCAGACTCTGAGATGAACAAACTCTATGAGAGAGTGAGG | subtyping |
| | AGGCAATTAAGGGAGAATGCCGAGGAGGATGGGACTGGATGTTTT | |
| | (SEQ ID NO: 879) | |
| 2k8_H16_majority- | TGGTCATACAATGCTAAACTTCTTGTACTGCTTGAAAATGGTAGAACAT | H |
| consensus | TAGACTTGCATGATGCAAATGTCAGAAACTTACATGATCAGGTCAAAA | subtyping |
| | GGGTGTTGAAGGACAATGCAATTGACGAAGGAAATGGTTGCTTC (SEQ | |
| | ID NO: 880) | |
| 2k8_N1_majority- | ATGAGGAATGCTCCTGTTATCCTGATTCTAGTGAAATCACATGTGTGTG | N |
| consensus | CAGGGATAACTGGCATGG (SEQ ID NO: 881) | subtyping |
| 2k8_N2_majority- | TCGAGGAGTGCTCTTGCTATCCTCGATATCCTGGTGTCAGATGTGTCTG | N |
| consensus | CAGAGACAACTGGAAAGG (SEQ ID NO: 882) | subtyping |
| 2k8_N3_majority- | TAGAAGAATGTTCCTGCTATGTGGACATTGATGTTTACTGTATATGTAG | N |
| consensus | GGACAATTGGAAAGG (SEQ ID NO: 883) | subtyping |
| 2k8_N4_majority- | TCGAAGAGTGTTCCTGTTACCCAAGTGGAACAGATATTGAGTGTGTCTG | N |
| consensus | TCGGGACAATTGGCGGGG (SEQ ID NO: 884) | subtyping |
| 2k8_N5_majority- | TTGAAGAGTGCTCTTGCTACCCCAACTTGGGTAAAGTGGAGTGTGTTTG | N |
| consensus | CCGAGATAATTGGAATGG (SEQ ID NO: 885) | subtyping |
| 2k8_N6_majority- | TAGAAGAATGCTCATGCTATGGAGCAGAAGAGGTGATCAAATGC | N |
| consensus | ATATGCAGGGACAATTGGAAAGG (SEQ ID NO: 886) | subtyping |
| 2k8_N7_majority- | TAGAGGAGTGCTCATGCTATGGGCACAATTCAAAGGTGACTTGTGTAT | N |
| consensus | GCAGGGACAACTGGCAAGG (SEQ ID NO: 887) | subtyping |
| 2k8_N8_majority- | TAGAAGAATGCTCATGCTACCCCAATGAAGGTAAAGTGGAATGTGTTT | N |
| consensus | GTAGGGACAACTGGACTGG (SEQ ID NO: 888) | subtyping |
| 2k8_N9_majority- | TTGAAGAATGCTCATGTTACGGGGAACGAACAGGAATTACCTGCACAT | N |
| consensus | GCAGGGACAATTGGCAGGG (SEQ ID NO: 889) | subtyping |
| 2k8v3_N1-u1- | ATGAGGAATGCTCCTGTTACCCAGACACTGGCATAGTGATGTGTGTAT | N sub- |
| g1 majority- | GCAGGGACAACTGGCATGG (SEQ ID NO: 890) | subtyping |
| consensus | | |
| 2k8v3_N1-u1- | ATGAGGAATGCTCCTGTTATCCTGATTCTAGTGAAATCACATGTGTGTG | N sub- |
| g2 majority- | CAGGGATAACTGGCATGG (SEQ ID NO: 891) | subtyping |
| consensus | | |
| 2k8v3_N1-u1- | ATGAGGAATGCTCATGTTATCCTGATACAGGCAAAGTAATGTGTGTTTG | N sub- |
| g3 majority- | CAGAGACAATTGGCATGC (SEQ ID NO: 892) | subtyping |
| consensus | | |
| 2k8v2_N2-u1- | TCGAGGAGTGCTCTTGTTATCCTCGATATCCTGGTGTCAGATGCGTCTG | N sub- |
| g1 majority- | CAGAGACAACTGGAAAGG (SEQ ID NO: 893) | subtyping |
| consensus | | |
| 2k8v2_N2-u1- | TCGAAGAGTGCTCTTGCTATCCTCGATATCCTGGTGTCAGATGTGTCTG | N sub- |
| g2 majority- | CAGAGACAACTGGAAAGG (SEQ ID NO: 894) | subtyping |
| consensus | | |
| 2k8v2_N2-u1- | TTGAGGARTGCTCCTGTTATCCTAGATATCCTGGTGTCAGATGTGTATG | N sub- |
| g3 majority- | CAGRGACAACTGGAAAGG (SEQ ID NO: 895) | subtyping |
| consensus | | |
| 2k8v2_N2-u1- | TTGAGGAGTGCTCCTGTTATCCTCGATTTCCTGGTGTCAGATGTGTCTG | N sub- |
| g4 majority- | CAGAGACAACTGGAAAGG (SEQ ID NO: 896) | subtyping |
| consensus | | |
| 2k8v2_N2-u1- | TAGAGGAGTGCTCCTGTTATCCCCGATATCCTGGTGTCAGATGCATCTG | N sub- |
| g5 majority- | TAGAGACAACTGGAAAGG (SEQ ID NO: 897) | subtyping |
| consensus | | |
| 2k8v2_N3-u1- | TAGAAGAATGTTCCTGCTATGTGGACATTGATGTTTACTGTATATGTAG | N sub- |
| g1 majority- | GGACAATTGGAAGGG (SEQ ID NO: 898) | subtyping |
| consensus | | |
| 2k8v2_N3-u1- | TAGAGGAGTGTTCTTGCTATGTGGACACCGATGTGTACTGCATATGTAG | N sub- |
| g2 majority- | GGACAATTGGAAAGG (SEQ ID NO: 899) | subtyping |
| consensus | | |
| 2k8v2_N3-u1- | TGGAAGAGTGTTCATGTTACACAGATGTAGACATCTACTGTGTGTGCA | N sub- |
| g3 majority- | GAGACAACTGGAAAGG (SEQ ID NO: 900) | subtyping |
| consensus | | |
| 2k8v2_N3-u1- | TGGAGGAGTGTTCTTGTTATGTGGACATCGATGTGTACTGCATATGTAG | N sub- |
| g4 majority- | GGACAATTGGAAAGG (SEQ ID NO: 901) | subtyping |
| consensus | | |
| 2k8v2_N4-u1- | TCGAAGAGTGTTCCTGTTACCCAAGTGGAACGGATATTGAGTGTGTCT | N sub- |
| g1 majority- | GTCGGGACAATTGGCGGGG (SEQ ID NO: 902) | subtyping |
| consensus | | |
| 2k8v2_N4-u1- | TCGAAGAGTGTTCCTGTTACCCGAGTGGAACAGATATTGAGTGTGTCT | N sub- |
| g2 majority- | GTCGGGACAATTGGCGGGG (SEQ ID NO: 903) | subtyping |
| consensus | | |
| 2k8v2_N4-u1- | TCGAAGAGTGTTCCTGTTACCCAAGTGGAATAGATATTGAGTGTGTCTG | N sub- |
| g3 majority- | TCGGGACAATTGGCGGGG (SEQ ID NO: 904) | subtyping |
| consensus | | |
| 2k8v2_N4-u1- | TTGAGGAGTGTTCCTGTTACCCAAGTGGAGAAAATGTCGAGTGTGTGT | N sub- |
| g4 majority- | GTAGAGACAATTGGAGAGG (SEQ ID NO: 905) | subtyping |
| consensus | | |
| 2k8v3_N5-u3- | TTGAAGAGTGCTCTTGCTACCCCAACTTGGGTAAAGTGGAGTGCGTTTG | N sub- |
| g1 majority- | CCGAGATAATTGGAATGG (SEQ ID NO: 906) | subtyping |
| consensus | | |
| 2k8v3_N5-u3- | TAGAGGAGTGTTCCTGTTACCCCAACATGGGAAAAGTGGAATGTGTTT | N sub- |
| g2 majority- | GCAGGGACAATTGGAATGG (SEQ ID NO: 907) | subtyping |
| consensus | | |
| 2k8v3_N5-u3- | TAGAGGAGTGTTCCTGTTATCCCAACATGGGGAAAGTGGAATGTGTTT | N sub- |
| g3 majority- | GCAGGGACAATTGGAACGG (SEQ ID NO: 908) | subtyping |
| consensus | | |
| 2k8v2_N6-u1- | TTGAAGAATGCTCATGCTATGGAGCAAAAGGAGTGATCAAATGCATCT | N sub- |
| g1 majority- | GCAGAGACAATTGGAAGGG (SEQ ID NO: 909) | subtyping |
| consensus | | |
| 2k8v2_N6-u1- | TAGAAGAGTGCTCATGCTATGGAGCAGAAGAAATGATTAAATGCATTT | N sub- |
| g2 majority- | GCAGGGATAATTGGAAGGG (SEQ ID NO: 910) | subtyping |
| consensus | | |
| 2k8v2_N6-u1- | TAGAAGAATGCTCGTGCTATGGAGCAGAAGAGGTGATTAAATGCATTT | N sub- |
| g3 majority- | GCAGGGACAATTGGAAAGG (SEQ ID NO: 911) | subtyping |
| consensus | | |
| 2k8v2_N6-u1- | TCGAAGAATGTTCATGCTATGGGGCAGCAGGGGTAATCAAATGTATAT | N sub- |
| g4 majority- | GCAGGGACAATTGGAAAGG (SEQ ID NO: 912) | subtyping |
| consensus | | |
| 2k8v2_N6-u1- | TCGAAGAGTGTTCATGCTACGGAGCAGCAGGGATGATCAAATGTGTAT | N sub- |
| g5 majority- | GCAGAGACAATTGGAAGGG (SEQ ID NO: 913) | subtyping |
| consensus | | |
| 2k8v2_N7-u1- | TTGAGGAATGCTCCTGTTACGGGCACAGTCAAAAGGTGACCTGTGTGT | N sub- |
| g1 majority- | GCAGAGATAACTGGCAGGG (SEQ ID NO: 914) | subtyping |
| consensus | | |
| 2k8v2_N7-u1- | TAGAGGAGTGCTCATGCTATGGGCACAATTCGAAGGTGACTTGTGTAT | N sub- |
| g2 majority- | GCAGGGACAACTGGCAAGG (SEQ ID NO: 915) | subtyping |
| consensus | | |
| 2k8v2_N7-u1- | TAGAGGAGTGCTCATGCTATGGGCACGATTCAAAAGTGACTTGTGTAT | N sub- |
| g3 majority- | GCAGGGACAACTGGCAAGG (SEQ ID NO: 916) | subtyping |
| consensus | | |
| 2k8v2_N7-u1- | TAGAGGAATGCTCATGCTATGGGCACAATTCAAAGGTGACTTGTGTAT | N sub- |
| g4 majority- | GCAGGGACAACTGGCAAGG (SEQ ID NO: 917) | subtyping |
| consensus | | |
| 2k8v2_N8-u1- | TAGAAGAATGCTCATGCTACCCCAATGAAGGTAAAGTGGAATGTGTTT | N sub- |
| g1 majority- | GTAGGGACAATTGGACTGG (SEQ ID NO: 918) | subtyping |
| consensus | | |
| 2k8v2_N8-u1- | TAGAAGAATGCTCATGCTACCCCAATGAAGGTAAAGTGGAGTGTGTTT | N sub- |
| g2 majority- | GTAGGGACAACTGGACTGG (SEQ ID NO: 919) | subtyping |
| consensus | | |
| 2k8v2_N8-u1- | TTGAGGAATGTTCTTGTTATCCAAATGATGGTAAAGTGGAATGCGTGT | N sub- |
| g3 majority- | GTAGAGACAACTGGACGGG (SEQ ID NO: 920) | subtyping |
| consensus | | |
| 2k8v2_N9-u1- | TTGAAGAATGCTCATGCTATGGGGTGCAGGCAGGTATTACTTGCACGT | N sub- |
| g1 majority- | GCAGGGATAATTGGCAGGG (SEQ ID NO: 921) | subtyping |
| consensus | | |
| 2k8v2_N9-u1- | TTGAAGAATGCTCATGCTACGGGGAACAAGCAGGTATTACTTGCACGT | N sub- |
| g2 majority- | GCAGGGATAATTGGCAGGG (SEQ ID NO: 922) | subtyping |
| consensus | | |
| 2k8v2_N9-u1- | TTGAAGAATGCTCATGTTACGGGGAACGAACAGGAATTACCTGCACAT | N sub- |
| g3 majority- | GCAGGGACAATTGGCAGGG (SEQ ID NO: 923) | subtyping |
| consensus | | |
| 2k8v2_N9-u1- | TTGAAGAATGCTCATGTTACGGGGAACGAACAGGGATTACCTGCACAT | N sub- |
| g4 majority- | GCAGGGACAATTGGCAGGG (SEQ ID NO: 924) | subtyping |
| consensus | | |

TABLE 8
Influenza crRNAs

| Full name | Design | Spacer sequence (RNA) | Hits majcon |
|---|---|---|---|
| 2k8v3_H1-u2-g1 | v3 | CAUUGUUUUUUAGUUGGCUUCUUACUUU | yes |
| | | (SEQ ID NO: 925) | |
| 2k8v3_H2-u1-g1 | v3 | CAUUAGAGUCAUGGAAAUCAAGUGUCCU | yes |
| | | (SEQ ID NO: 926) | |
| 2k8v3_H3-u2-g1 | v3 | UGUAUGUUGGUUCUCCAGGGCAACAAGA | yes |
| | | (SEQ ID NO: 927) | |
| 2k8v1_H4-u1-g1 | v1 | UAGUAUGUUGAUUUUCUAAUGCCACCAG | yes |
| | | (SEQ ID NO: 928) | |
| 2k8v3_H5-u2-g1 | v3 | CAGCUCUUUUGCAUUAUCCUUAAGCUGU | yes |
| | | (SEQ ID NO: 929) | |
| 2k8v3_H6-u1-g2 | v3 | GGUCAUUAGCAUUGUCCCUUAGUUGUGA | yes |
| | | (SEQ ID NO: 930) | |
| 2k8v4_H7-u1-g1 | v4 | UCUCCAUCGCUAUCAAGAGUUCAGCGUU | yes |
| | | (SEQ ID NO: 931) | |
| 2k8v4_H8-u1-g1 | v4 | UCACUUCAUCAAAGAGGUUCUUGACAUU | yes |
| | | (SEQ ID NO: 932) | |
| 2k8v1_H9-u1-g3 | v1 | UUGCGUCAUGCUCAUCGAGUGUUUUCUG | yes |
| | | (SEQ ID NO: 933) | |
| 2k8v4_H10-u1-g1 | v4 | GAUUCAGCAUUUCUGAAUCAGCCAUGUC | yes |
| | | (SEQ ID NO: 934) | |
| 2k8v3_H11-u2-g1 | v3 | CAUUCGUCUGACCUUUUCAUGGAGGUUU | yes |
| | | (SEQ ID NO: 935) | |
| 2k8v2_H12-u1-g2 | | UAAUGUCUUCUGAUUUUCCAAUAGAACA | yes |
| | v2 | (SEQ ID NO: 936) | |
| 2k8v3_H13-u1-g2 | v3 | UGCAUGUCUAGAGUCUUGUCGUUCUCUA | yes |
| | | (SEQ ID NO: 937) | |
| 2k8v4_H14-u1-g1 | v4 | GAUCUUCCGCAUUUUCCCUAAGUUGUCU | yes |
| | | (SEQ ID NO: 938) | |
| 2k8v3_H15-u2-g1 | v3 | AGAGUCUGCAAGGUCAAUUGUGUGUUGA | yes |
| | | (SEQ ID NO: 939) | |
| 2k8v3_H16-u5-g1 | v3 | UCGUGCAAGUCUAAUGUUCUACCAUUUU | yes |
| | | (SEQ ID NO: 940) | |
| 2k8v3_N1-u1-g1 | v3 | UCACUAUGCCAGUGUCUGGGUAACAGGA | no, but used |
| | | (SEQ ID NO: 941) | for seedstock |
| | | | subtyping |
| 2k8v3_N1-u1-g2 | v3 | UGAUUUCACUAGAAUCAGGAUAACAGGA | yes |
| | | (SEQ ID NO: 942) | |
| 2k8v3_N1-u1-g3 | v3 | ACAUUACUUUGCCUGUAUCAGGAUAACA | |
| | | (SEQ ID NO: 943) | |
| 2k8v2_N2-u1-g1 | v2 | CAUCUGACACCAGGAUAUCGAGGAUAAC | |
| | | (SEQ ID NO: 944) | |
| 2k8v2_N2-u1-g2 | v2 | CACAUCUGACACCAGGAUAUCGAGGAUA | yes |
| | | (SEQ ID NO: 945) | |
| 2k8v2_N 2-u1-g3 | v2 | UACACAUCUGACACCAGGAUACUUAGGA | |
| | | (SEQ ID NO: 946) | |
| 2k8v2_N 2-u1-g4 | v2 | GACACAUCUGACACCAGGAGAUCGAGGA | |
| | | (SEQ ID NO: 947) | |
| 2k8v2_N 2-u1-g5 | v2 | GCAUCUGACACCAGGAUAUCGGGGAUAA | |
| | | (SEQ ID NO: 948) | |
| 2k8v2_N3-u1-g1 | v2 | AUACAGUAAACAUCAAUGUCCACAUAGC | yes |
| | | (SEQ ID NO: 949) | |
| 2k8v2_N3-u1-g2 | v2 | CCUACAUAUGCAGUACACAUCGGUGUCC | |
| | | (SEQ ID NO: 950) | |
| 2k8v2_N3-u1-g3 | v2 | ACACAGUAGAUGUCUACAUCUGUGUAAC | |
| | | (SEQ ID NO: 951) | |
| 2k8v2_N3-u1-g4 | v2 | AUACAAUACACAUCAAUGUCCACAUAAC | |
| | | (SEQ ID NO: 952) | |
| 2k8v2_N4-u1-g1 | v2 | GACAGACACACUCAAUAUCCGUUCCACU | |
| | | (SEQ ID NO: 953) | |
| 2k8v2_N4-u1-g2 | v2 | CAGACACACUCAAUAUCUGUUCCACUUG4 | yes |
| | | (SEQ ID NO: 954) | |
| 2k8v2_N4-u1-g3 | v2 | ACACUCAAUAUUUAUUCCACUUGGGUAA | |
| | | (SEQ ID NO: 955) | |
| 2k8v2_N4-u1-g4 | v2 | ACACUCGACAUUUUCUCCACUUGGGUAA | |
| | | (SEQ ID NO: 956) | |
| 2k8v3_N5-u3-g1 | v3 | GGCAAACGCACUCCACUUUACCCAAGUU | yes |
| | | (SEQ ID NO: 957) | |
| 2k8v3_N5-u3-g2 | v3 | CACAUUCCACUUUUCCCAUGUUGGGGUA | |
| | | (SEQ ID NO: 958) | |
| 2k8v3_N5-u3-g3 | v3 | ACAUUCCACUUUCCCCAUGUUGGGAUAA | |
| | | (SEQ ID NO: 959) | |
| 2k8v2_N6-u1-g1 | v2 | CAUUUGAUCACUCCUUUUGCUCCAUAGC | |
| | | (SEQ ID NO: 960) | |
| 2k8v2_N6-u1-g2 | v2 | CAUUUAAUCAUUUCUUCUGCUCCAUAGC | |
| | | (SEQ ID NO: 961) | |
| 2k8v2_N6-u1-g3 | v2 | CAUUUAAUCACCUCUUCUGCUCCAUAGC | yes |
| | | (SEQ ID NO: 962) | |
| 2k8v2_N6-u1-g4 | v2 | CAUUUGAUUACCCCUGCUGCCCCAUAGC | |
| | | (SEQ ID NO: 963) | |
| 2k8v2_N6-u1-g5 | v2 | AUACACAUUUGAUCAUCCCUGCUGCUCC | |
| | | (SEQ ID NO: 964) | |
| 2k8v2_N7-u1-g1 | v2 | ACACAGGUCACCUUUUGACUGUGCCCGU | |
| | | (SEQ ID NO: 965) | |
| 2k8v2_N7-u1-g2 | v2 | CAAGUCACCUUCGAAUUGUGCCCAUAGC | |
| | | (SEQ ID NO: 966) | |
| 2k8v2_N7-u1-g3 | v2 | CAAGUCACUUUUGAAUCGUGCCCAUAGC | |
| | | (SEQ ID NO: 967) | |
| 2k8v2_N7-u1-g4 | v2 | CAUACACAAGUCACCUUUGAAUUGUGCC | yes |
| | | (SEQ ID NO: 968) | |
| 2k8v2_N8-u1-g1 | v2 | ACAAACACAUUCCACUUUACCUUCAUUG | yes |
| | | (SEQ ID NO: 969) | |
| 2k8v2_N8-u1-g2 | v2 | ACAAACACACUCCACUUUACCUUCAUUG | |
| | | (SEQ ID NO: 970) | |
| 2k8v2_N8-u1-g3 | v2 | ACACGCAUUCCACUUUACCAUCAUUUGG | |
| | | (SEQ ID NO: 971) | |
| 2k8v2_N9-u1-g1 | v2 | GUGCAAGUAAUACCUGCCUGCACCCCAU | |
| | | (SEQ ID NO: 972) | |
| 2k8v2_N9-u1-g2 | v2 | ACGUGCAAGUAAUACCUGCUUGUUCCCC | |
| | | (SEQ ID NO: 973) | |
| 2k8v2_N9-u1-g3 | v2 | UGCAGGUAAUUCCUGUUCGUUCCCCGUA | yes |
| | | (SEQ ID NO: 974) | |
| 2k8v2_N9-u1-g4 | v2 | GCAGGUAAUCCCUGUUCGUUUCCCGUAA | |
| | | (SEQ ID NO: 975) | |

### HIV DRM Panel Design

Primer design: Applicants used a primer pooling strategy in which primer pairs were divided into overlapping “odd” and “even” primer pools based on the locations of DRMs within the reverse transcriptase and integrase genes. This allowed for all mutations to be contained in at least one amplicon, without creating any issues during amplification. Primer sequences were designed using primer3 v2.4.0 with the following parameters: PRIMER_PRODUCT_OPT_SIZE=150, PRIMER_MAX_GC=70, PRIMER_MIN_GC=30, PRIMER_OPT_GC_PERCENT=50, PRIMER_MIN_TM=55, PRIMER_MAX_TM=60, PRIMER_DNA_CONC=150, PRIMER_OPT_SIZE=20, PRIMER_MIN_SIZE=16, PRIMER_MAX_SIZE=29. Amplicon lengths ranged between 150 and 250 nucleotides. All primer sequences are in Table 9.

crRNA design: Pairs of crRNAs were designed for HIV DRM identification using three different strategies: mutation on position 3 and synthetic mismatch on position 5, DRM codon on positions 3-5 and synthetic mismatch on position 6, and DRM codon on positions 4-6 with synthetic mismatch at position 3. Sequences were designed based on the HIV subtype B consensus sequence, using the most-commonly used codons for each respective amino acid. All designs were experimentally tested, and the best-performing design was chosen for the final panel.

TABLE 9
HIV

| Type | Identity | Sequence |
|---|---|---|
| Primer | HIVRT 1-Fwd | gaaatTAATACGACTCACTATAgggAATTAAAGCCAGGAATGGATG (SEQ ID |
| | | NO: 976) |
| Primer | HIVRT 1-Rev | AGTCTTGAGTTCTCTTATTAAGTTC (SEQ ID NO: 977) |
| Primer | HIVRT 2-Fwd | gaaatTAATACGACTCACTATAgggAGAGAACTCAAGACTTCTGG (SEQ ID |
| | | NO: 978) |
| Primer | HIVRT 2-Rev | TGGTAAATGCAGTATACTTCCTGA (SEQ ID NO: 979) |
| Primer | HIVRT 3-Fwd | gaaatTAATACGACTCACTATAgggTCCCTTAGATAAAGACTTCAGGA (SEQ ID |
| | | NO: 980) |
| Primer | HIVRT 3-Rev | TGTCATGCTACTTTGGAATATTGC (SEQ ID NO: 981) |
| Primer | HIVRT 4-Fwd | gaaatTAATACGACTCACTATAgggTCCAAAGTAGCATGACAAAAATCT (SEQ ID |
| | | NO: 982) |
| Primer | HIVRT 4-Rev | ACAGATGTTGTCTCAGTTCCTC (SEQ ID NO: 983) |
| Primer | HIVIN 1-Fwd | gaaatTAATACGACTCACTATAgggAGAAATAGTAGCCAGCTGTGA (SEQ ID |
| | | NO: 984) |
| Primer | HIVIN 1-Rev | CACTGGCTACATGAACTGCT (SEQ ID NO: 985) |
| Primer | HIVIN 2-Fwd | gaaatTAATACGACTCACTATAgggCAGTTCATGTAGCCAGTGGA (SEQ ID |
| | | NO: 986) |
| Primer | HIVIN 2-Rev | AATTCCTGCTTGATCCCTGC (SEQ ID NO: 987) |
| Primer | HIVIN 3-Fwd | gaaatTAATACGACTCACTATAgggCCAGTACTACGGTTAAGGCC (SEQ ID |
| | | NO: 988) |
| Primer | HIVIN 3-Rev | GCTGTCTTAAGATGTTCAGCCT (SEQ ID NO: 989) |
| Primer | HIVIN 4-Fwd | gaaatTAATACGACTCACTATAgggAGCAACAGACATACAAACTAAAGA (SEQ |
| | | ID NO: 990) |
| Primer | HIVIN 4-Rev | TCCATAATCCCTAATGATCTTTGC (SEQ ID NO: 991) |
| crRNA | HIVRT-K65R- | UUUUUGUUUAUGGCAAAUACUGGAGUAU (SEQ ID NO: 992) |
| | ancestral-v1 | |
| crRNA | HIVRT-K65R- | UUUCUGUUUAUGGCAAAUACUGGAGUAU (SEQ ID NO: 993) |
| | derived-v1 | |
| crRNA | HIVRT-K103N- | UUUUUGUUUUUUAACCCUGCGGGAUGUG (SEQ ID NO: 994) |
| | ancestral-v1 | |
| crRNA | HIVRT-K103N- | UUGUUGUUUUUUAACCCUGCGGGAUGUG (SEQ ID NO: 995) |
| | derived-v1 | |
| crRNA | HIVRT- | GUUACAGAUUUUUUCUUUUUUAACCCUG (SEQ ID NO: 996) |
| | V106M- | |
| | ancestral-v1 | |
| crRNA | HIVRT- | GUCAUAGAUUUUUUCUUUUUUAACCCUG (SEQ ID NO: 997) |
| | V106M- | |
| | derived-v1 | |
| crRNA | HIVRT-Y181C- | GAUACAUAACUAUGUCUGGAUUUUGUUU (SEQ ID NO: 998) |
| | ancestral-v0 | |
| crRNA | HIVRT-Y181C- | GACACAUAACUAUGUCUGGAUUUUGUUU (SEQ ID NO: 999) |
| | derived-v0 | |
| crRNA | HIVRT- | AUGCAUGUAUUGAUAGAUAACUAUGUCU (SEQ ID NO: 1000) |
| | M184V- | |
| | ancestral-v2 | |
| crRNA | HIVRT-M184V- | AUGCACGUAUUGAUAGAUAACUAUGUCU (SEQ ID NO: 1001) |
| | derived-v2 | |
| crRNA | HIVRT-G190A- | GAUCCAACAUACAAAUCAUCCAUGUAUU (SEQ ID NO: 1002) |
| | ancestral-v1 | |
| crRNA | HIVRT-G190A- | GAUGCAACAUACAAAUCAUCCAUGUAUU (SEQ ID NO: 1003) |
| | derived-v1 | |
| crRNA | HIVIN-66A- | AUCUGUACAAUCUAGUUGCCAUAUUCCU (SEQ ID NO: 1004) |
| | ancestral-v2 | |
| crRNA | HIVIN-66A- | AUCUGCACAAUCUAGUUGCCAUAUUCCU (SEQ ID NO: 1005) |
| | derived-v2 | |
| crRNA | HIVIN-661- | AUCUGUACAAUCUAGUUGCCAUAUUCCU (SEQ ID NO: 1006) |
| | ancestral-v2 | |
| crRNA | HIVIN-661- | AUCUAUACAAUCUAGUUGCCAUAUUCCU (SEQ ID NO: 1007) |
| | derived-v2 | |
| crRNA | HIVIN-66K- | AUCUGUACAAUCUAGUUGCCAUAUUCCU (SEQ ID NO: 1008) |
| | ancestral-v2 | |
| crRNA | HIVIN-66K- | AUCUUUACAAUCUAGUUGCCAUAUUCCU (SEQ ID NO: 1009) |
| | derived-v2 | |
| crRNA | HIVIN-74M- | ACCAGCAUAAUUUUUCCUUCUAAAUGUG (SEQ ID NO: 1010) |
| | ancestral-v1 | |
| crRNA | HIVIN-74M- | ACCAUCAUAAUUUUUCCUUCUAAAUGUG (SEQ ID NO: 1011) |
| | derived-v1 | |
| crRNA | HIVIN-92G- | UCUCAGCUGGAAUAACUUCUGCUUCUAU (SEQ ID NO: 1012) |
| | ancestral-v4 | |
| crRNA | HIVIN-92G- | UCCCAGCUGGAAUAACUUCUGCUUCUAU (SEQ ID NO: 1013) |
| | derived-v4 | |
| crRNA | HIVIN-92Q- | UGUCUCUGCUGGAAUAACUUCUGCUUCU (SEQ ID NO: 1014) |
| | ancestral-v2 | |
| crRNA | HIVIN-92Q- | UGUCUGUGCUGGAAUAACUUCUGCUUCU (SEQ ID NO: 1015) |
| | derived-v2 | |
| crRNA | HIVIN-97A- | UGGUGUUUCCUGCCCUGUCUCUGCUGGA (SEQ ID NO: 1016) |
| | ancestral-v2 | |
| crRNA | HIVIN-97A- | UGGUGCUUCCUGCCCUGUCUCUGCUGGA (SEQ ID NO: 1017) |
| | derived-v2 | |
| crRNA | HIVIN-121Y- | UGAAUUUGCUGCCAUUGUCUGUAUGUAU (SEQ ID NO: 1018) |
| | ancestral-v0 | |
| crRNA | HIVIN-121Y- | UGUAUUUGCUGCCAUUGUCUGUAUGUAU (SEQ ID NO: 1019) |
| | derived-v0 | |
| crRNA | HIVIN-138A- | AUUCGUGCUUGAUCCCUGCCCACCAACA (SEQ ID NO: 1020) |
| | ancestral-v0 | |
| crRNA | HIVIN-138A- | AUGCGUGCUUGAUCCCUGCCCACCAACA (SEQ ID NO: 1021) |
| | derived-v0 | |
| crRNA | HIVIN-138K- | AAUUCGUGCUUGAUCCCUGCCCACCAAC (SEQ ID NO: 1022) |
| | ancestral-v1 | |
| crRNA | HIVIN-138K- | AAUUUGUGCUUGAUCCCUGCCCACCAAC (SEQ ID NO: 1023) |
| | derived-v1 | |
| crRNA | HIVIN-140A- | UGCCUAAUUCCUGCUUGAUCCCUGCCCA (SEQ ID NO: 1024) |
| | ancestral-v0 | |
| crRNA | HIVIN-140A- | UGGCUAAUUCCUGCUUGAUCCCUGCCCA (SEQ ID NO: 1025) |
| | derived-v0 | |
| crRNA | HIVIN-140S- | AAAGCCAAAUUCCUGCUUGAUCCCUGCC (SEQ ID NO: 1026) |
| | ancestral-v2 | |
| crRNA | HIVIN-140S- | AAAGCUAAAUUCCUGCUUGAUCCCUGCC (SEQ ID NO: 1027) |
| | derived-v2 | |
| crRNA | HIVIN-143C- | UGUACGGAAUGCCAAAUUCCUGCUUGAU (SEQ ID NO: 1028) |
| | ancestral-v0 | |
| crRNA | HIVIN-143C- | UGCACGGAAUGCCAAAUUCCUGCUUGAU (SEQ ID NO: 1029) |
| | derived-v0 | |
| crRNA | HIVIN-143H- | UUGUACGGAAUGCCAAAUUCCUGCUUGA (SEQ ID NO: 1030) |
| | ancestral-v1 | |
| crRNA | HIVIN-143H- | UUGUGCGGAAUGCCAAAUUCCUGCUUGA (SEQ ID NO: 1031) |
| | derived-v1 | |
| crRNA | HIVIN-143R- | UGUAGGGAAUGCCAAAUUCCUGCUUGAU (SEQ ID NO: 1032) |
| | ancestral-v0 | |
| crRNA | HIVIN-143R- | UGCGGGGAAUGCCAAAUUCCUGCUUGAU (SEQ ID NO: 1033) |
| | derived-v0 | |
| crRNA | HIVIN-147G- | UGACUAUGGGGAUUGUAGGGAAUGCCAA (SEQ ID NO: 1034) |
| | ancestral-v1 | |
| crRNA | HIVIN-147G- | UGACCAUGGGGAUUGUAGGGAAUGCCAA (SEQ ID NO: 1035) |
| | derived-v1 | |
| crRNA | HIVIN-148H- | CCUUGUCUUUGGGGAUUGUAGGGAAUGC (SEQ ID NO: 1036) |
| | ancestral-v1 | |
| crRNA | HIVIN-148H- | CCGUGUCUUUGGGGAUUGUAGGGAAUGC (SEQ ID NO: 1037) |
| | derived-v1 | |
| crRNA | HIVIN-148K- | CCUUGUCUUUGGGGAUUGUAGGGAAUGC (SEQ ID NO: 1038) |
| | ancestral-v1 | |
| crRNA | HIVIN-148K- | CCUUUUCUUUGGGGAUUGUAGGGAAUGC (SEQ ID NO: 1039) |
| | derived-v1 | |
| crRNA | HIVIN-148R- | CCUUGUCUUUGGGGAUUGUAGGGAAUGC (SEQ ID NO: 1040) |
| | ancestral-v1 | |
| crRNA | HIVIN-148R- | CCUCGUCUUUGGGGAUUGUAGGGAAUGC (SEQ ID NO: 1041) |
| | derived-v1 | |
| crRNA | HIVIN-155H- | UUAUUGAUAGAUUCUACUACUCCUUGAC (SEQ ID NO: 1042) |
| | ancestral-v1 | |
| crRNA | HIVIN-155H- | UUAUGGAUAGAUUCUACUACUCCUUGAC (SEQ ID NO: 1043) |
| | derived-v1 | |
| crRNA | HIVIN-263K- | UUUCUACUUGGCACUACUUUUAUGU (SEQ ID NO: 1044) |
| | ancestral-vAlt | |
| crRNA | HIVIN-263K- | UUUUUACUUGGCACUACUUUUAUGU (SEQ ID NO: 1045) |
| | derived-vAlt | |
| gBlock | HIVRT | gaaatTAATACGACTCACTATAgggCCCATTAGTCCTATTGAAACTGTACCAGTA |
| | Reference | AAATTAAAGCCAGGAATGGATGGCCCAAAAGTTAAACAATGGCCATTGACAG |
| | | AAGAAAAAATAAAAGCATTAGTAGAAATTTGTACAGAAATGGAAAAGGAAG |
| | | GGAAAATTTCAAAAATTGGGCCTGAAAATCCATACAATACTCCAGTATTTGCC |
| | | ATAAAGAAAAAAGACAGTACTAAATGGAGAAAATTAGTAGATTTCAGAGAAC |
| | | TTAATAAGAGAACTCAAGACTTCTGGGAAGTTCAATTAGGAATACCACATCCC |
| | | GCAGGGTTAAAAAAGAAAAAATCAGTAACAGTACTGGATGTGGGTGATGCAT |
| | | ATTTTTCAGTTCCCTTAGATAAAGACTTCAGGAAGTATACTGCATTTACCATAC |
| | | CTAGTATAAACAATGAGACACCAGGGATTAGATATCAGTACAATGTGCTTCCA |
| | | CAGGGATGGAAAGGATCACCAGCAATATTCCAAAGTAGCATGACAAAAATCT |
| | | TAGAGCCTTTTAGAAAACAAAATCCAGACATAGTTATCTATCAATACATGGAT |
| | | GATTTGTATGTAGGATCTGACTTAGAAATAGGGCAGCATAGAACAAAAATAG |
| | | AGGAACTGAGACAACATCTGTTGAGGTGGGGATTTACCACACCAGACAAAAA |
| | | ACATCAGAAAGAACCTCCATTCCTTTGGATGGGTTATGAAC (SEQ ID |
| | | NO: 1046) |
| gBlock | HIVRT K65R | gaaatTAATACGACTCACTATAgggCCCATTAGTCCTATTGAAACTGTACCAGTA |
| | | AAATTAAAGCCAGGAATGGATGGCCCAAAAGTTAAACAATGGCCATTGACAG |
| | | AAGAAAAAATAAAAGCATTAGTAGAAATTTGTACAGAAATGGAAAAGGAAG |
| | | GGAAAATTTCAAAAATTGGGCCTGAAAATCCATACAATACTCCAGTATTTGCC |
| | | ATAAAGAGAAAAGACAGTACTAAATGGAGAAAATTAGTAGATTTCAGAGAAC |
| | | TTAATAAGAGAACTCAAGACTTCTGGGAAGTTCAATTAGGAATACCACATCCC |
| | | GCAGGGTTAAAAAAGAAAAAATCAGTAACAGTACTGGATGTGGGTGATGCAT |
| | | ATTTTTCAGTTCCCTTAGATAAAGACTTCAGGAAGTATACTGCATTTACCATAC |
| | | CTAGTATAAACAATGAGACACCAGGGATTAGATATCAGTACAATGTGCTTCCA |
| | | CAGGGATGGAAAGGATCACCAGCAATATTCCAAAGTAGCATGACAAAAATCT |
| | | TAGAGCCTTTTAGAAAACAAAATCCAGACATAGTTATCTATCAATACATGGAT |
| | | GATTTGTATGTAGGATCTGACTTAGAAATAGGGCAGCATAGAACAAAAATAG |
| | | AGGAACTGAGACAACATCTGTTGAGGTGGGGATTTACCACACCAGACAAAAA |
| | | ACATCAGAAAGAACCTCCATTCCTTTGGATGGGTTATGAAC (SEQ ID |
| | | NO: 1047) |
| gBlock | HIVRT K103N | gaaatTAATACGACTCACTATAgggCCCATTAGTCCTATTGAAACTGTACCAGTA |
| | | AAATTAAAGCCAGGAATGGATGGCCCAAAAGTTAAACAATGGCCATTGACAG |
| | | AAGAAAAAATAAAAGCATTAGTAGAAATTTGTACAGAAATGGAAAAGGAAG |
| | | GGAAAATTTCAAAAATTGGGCCTGAAAATCCATACAATACTCCAGTATTTGCC |
| | | ATAAAGAAAAAAGACAGTACTAAATGGAGAAAATTAGTAGATTTCAGAGAAC |
| | | TTAATAAGAGAACTCAAGACTTCTGGGAAGTTCAATTAGGAATACCACATCCC |
| | | GCAGGGTTAAAAAAGAACAAATCAGTAACAGTACTGGATGTGGGTGATGCAT |
| | | ATTTTTCAGTTCCCTTAGATAAAGACTTCAGGAAGTATACTGCATTTACCATAC |
| | | CTAGTATAAACAATGAGACACCAGGGATTAGATATCAGTACAATGTGCTTCCA |
| | | CAGGGATGGAAAGGATCACCAGCAATATTCCAAAGTAGCATGACAAAAATCT |
| | | TAGAGCCTTTTAGAAAACAAAATCCAGACATAGTTATCTATCAATACATGGAT |
| | | GATTTGTATGTAGGATCTGACTTAGAAATAGGGCAGCATAGAACAAAAATAG |
| | | AGGAACTGAGACAACATCTGTTGAGGTGGGGATTTACCACACCAGACAAAAA |
| | | ACATCAGAAAGAACCTCCATTCCTTTGGATGGGTTATGAAC (SEQ ID |
| | | NO: 1048) |
| gBlock | HIVRT V106M | gaaatTAATACGACTCACTATAgggCCCATTAGTCCTATTGAAACTGTACCAGTA |
| | | AAATTAAAGCCAGGAATGGATGGCCCAAAAGTTAAACAATGGCCATTGACAG |
| | | AAGAAAAAATAAAAGCATTAGTAGAAATTTGTACAGAAATGGAAAAGGAAG |
| | | GGAAAATTTCAAAAATTGGGCCTGAAAATCCATACAATACTCCAGTATTTGCC |
| | | ATAAAGAAAAAAGACAGTACTAAATGGAGAAAATTAGTAGATTTCAGAGAAC |
| | | TTAATAAGAGAACTCAAGACTTCTGGGAAGTTCAATTAGGAATACCACATCCC |
| | | GCAGGGTTAAAAAAGAAAAAATCAATGACAGTACTGGATGTGGGTGATGCAT |
| | | ATTTTTCAGTTCCCTTAGATAAAGACTTCAGGAAGTATACTGCATTTACCATAC |
| | | CTAGTATAAACAATGAGACACCAGGGATTAGATATCAGTACAATGTGCTTCCA |
| | | CAGGGATGGAAAGGATCACCAGCAATATTCCAAAGTAGCATGACAAAAATCT |
| | | TAGAGCCTTTTAGAAAACAAAATCCAGACATAGTTATCTATCAATACATGGAT |
| | | GATTTGTATGTAGGATCTGACTTAGAAATAGGGCAGCATAGAACAAAAATAG |
| | | AGGAACTGAGACAACATCTGTTGAGGTGGGGATTTACCACACCAGACAAAAA |
| | | ACATCAGAAAGAACCTCCATTCCTTTGGATGGGTTATGAAC (SEQ ID |
| | | NO: 1049) |
| gBlock | HIVRT Y181C | gaaatTAATACGACTCACTATAgggCCCATTAGTCCTATTGAAACTGTACCAGTA |
| | | AAATTAAAGCCAGGAATGGATGGCCCAAAAGTTAAACAATGGCCATTGACAG |
| | | AAGAAAAAATAAAAGCATTAGTAGAAATTTGTACAGAAATGGAAAAGGAAG |
| | | GGAAAATTTCAAAAATTGGGCCTGAAAATCCATACAATACTCCAGTATTTGCC |
| | | ATAAAGAAAAAAGACAGTACTAAATGGAGAAAATTAGTAGATTTCAGAGAAC |
| | | TTAATAAGAGAACTCAAGACTTCTGGGAAGTTCAATTAGGAATACCACATCCC |
| | | GCAGGGTTAAAAAAGAAAAAATCAGTAACAGTACTGGATGTGGGTGATGCAT |
| | | ATTTTTCAGTTCCCTTAGATAAAGACTTCAGGAAGTATACTGCATTTACCATAC |
| | | CTAGTATAAACAATGAGACACCAGGGATTAGATATCAGTACAATGTGCTTCCA |
| | | CAGGGATGGAAAGGATCACCAGCAATATTCCAAAGTAGCATGACAAAAATCT |
| | | TAGAGCCTTTTAGAAAACAAAATCCAGACATAGTTATCTGTCAATACATGGAT |
| | | GATTTGTATGTAGGATCTGACTTAGAAATAGGGCAGCATAGAACAAAAATAG |
| | | AGGAACTGAGACAACATCTGTTGAGGTGGGGATTTACCACACCAGACAAAAA |
| | | ACATCAGAAAGAACCTCCATTCCTTTGGATGGGTTATGAAC (SEQ ID |
| | | NO: 1050) |
| gBlock | HIVRT M184V | gaaatTAATACGACTCACTATAgggCCCATTAGTCCTATTGAAACTGTACCAGTA |
| | | AAATTAAAGCCAGGAATGGATGGCCCAAAAGTTAAACAATGGCCATTGACAG |
| | | AAGAAAAAATAAAAGCATTAGTAGAAATTTGTACAGAAATGGAAAAGGAAG |
| | | GGAAAATTTCAAAAATTGGGCCTGAAAATCCATACAATACTCCAGTATTTGCC |
| | | ATAAAGAAAAAAGACAGTACTAAATGGAGAAAATTAGTAGATTTCAGAGAAC |
| | | TTAATAAGAGAACTCAAGACTTCTGGGAAGTTCAATTAGGAATACCACATCCC |
| | | GCAGGGTTAAAAAAGAAAAAATCAGTAACAGTACTGGATGTGGGTGATGCAT |
| | | ATTTTTCAGTTCCCTTAGATAAAGACTTCAGGAAGTATACTGCATTTACCATAC |
| | | CTAGTATAAACAATGAGACACCAGGGATTAGATATCAGTACAATGTGCTTCCA |
| | | CAGGGATGGAAAGGATCACCAGCAATATTCCAAAGTAGCATGACAAAAATCT |
| | | TAGAGCCTTTTAGAAAACAAAATCCAGACATAGTTATCTATCAATACGTGGAT |
| | | GATTTGTATGTAGGATCTGACTTAGAAATAGGGCAGCATAGAACAAAAATAG |
| | | AGGAACTGAGACAACATCTGTTGAGGTGGGGATTTACCACACCAGACAAAAA |
| | | ACATCAGAAAGAACCTCCATTCCTTTGGATGGGTTATGAAC (SEQ ID |
| | | NO: 1051) |
| gBlock | HIVRT G190A | gaaatTAATACGACTCACTATAgggCCCATTAGTCCTATTGAAACTGTACCAGTA |
| | | AAATTAAAGCCAGGAATGGATGGCCCAAAAGTTAAACAATGGCCATTGACAG |
| | | AAGAAAAAATAAAAGCATTAGTAGAAATTTGTACAGAAATGGAAAAGGAAG |
| | | GGAAAATTTCAAAAATTGGGCCTGAAAATCCATACAATACTCCAGTATTTGCC |
| | | ATAAAGAAAAAAGACAGTACTAAATGGAGAAAATTAGTAGATTTCAGAGAAC |
| | | TTAATAAGAGAACTCAAGACTTCTGGGAAGTTCAATTAGGAATACCACATCCC |
| | | GCAGGGTTAAAAAAGAAAAAATCAGTAACAGTACTGGATGTGGGTGATGCAT |
| | | ATTTTTCAGTTCCCTTAGATAAAGACTTCAGGAAGTATACTGCATTTACCATAC |
| | | CTAGTATAAACAATGAGACACCAGGGATTAGATATCAGTACAATGTGCTTCCA |
| | | CAGGGATGGAAAGGATCACCAGCAATATTCCAAAGTAGCATGACAAAAATCT |
| | | TAGAGCCTTTTAGAAAACAAAATCCAGACATAGTTATCTATCAATACATGGAT |
| | | GATTTGTATGTAGCATCTGACTTAGAAATAGGGCAGCATAGAACAAAAATAG |
| | | AGGAACTGAGACAACATCTGTTGAGGTGGGGATTTACCACACCAGACAAAAA |
| | | ACATCAGAAAGAACCTCCATTCCTTTGGATGGGTTATGAAC (SEQ ID |
| | | NO: 1052) |
| gBlock | HIVIN | gaaatTAATACGACTCACTATAgggAGCAAAAGAAATAGTAGCCAGCTGTGATA |
| | Reference | AATGTCAGCTAAAAGGAGAAGCCATGCATGGACAAGTAGACTGTAGTCCAGG |
| | | AATATGGCAACTAGATTGTACACATTTAGAAGGAAAAATTATCCTGGTAGCAG |
| | | TTCATGTAGCCAGTGGATATATAGAAGCAGAAGTTATTCCAGCAGAGACAGG |
| | | GCAGGAAACAGCATACTTTCTCTTAAAATTAGCAGGAAGATGGCCAGTAAAA |
| | | ACAATACATACAGACAATGGCAGCAATTTCACCAGTACTACGGTTAAGGCCGC |
| | | CTGTTGGTGGGCAGGGATCAAGCAGGAATTTGGCATTCCCTACAATCCCCAA |
| | | AGTCAAGGAGTAGTAGAATCTATGAATAAAGAATTAAAGAAAATTATAGGAC |
| | | AGGTAAGAGATCAGGCTGAACATCTTAAGACAGCAGTACAAATGGCAGTATT |
| | | CATCCACAATTTTAAAAGAAAAGGGGGGATTGGGGGGTACAGTGCAGGGGA |
| | | AAGAATAGTAGACATAATAGCAACAGACATACAAACTAAAGAATTACAAAAA |
| | | CAAATTACAAAAATTCAAAATTTTCGGGTTTATTACAGGGACAGCAGAGATCC |
| | | ACTTTGGAAAGGACCAGCAAAGCTTCTCTGGAAAGGTGAAGGGGCAGTAGTA |
| | | ATACAAGATAATAGTGACATAAAAGTAGTGCCAAGAAGAAAAGCAAAGATCA |
| | | TTAGGGATTATGGAAAAC (SEQ ID NO: 1053) |
| gBlock | HIVIN 66A- | gaaatTAATACGACTCACTATAgggAGCAAAAGAAATAGTAGCCAGCTGTGATA |
| | 92G-138K- | AATGTCAGCTAAAAGGAGAAGCCATGCATGGACAAGTAGACTGTAGTCCAGG |
| | 148K | AATATGGCAACTAGATTGTGCACATTTAGAAGGAAAAATTATCCTGGTAGCAG |
| | | TTCATGTAGCCAGTGGATATATAGAAGCAGAAGTTATTCCAGCAGGGACAGG |
| | | GCAGGAAACAGCATACTTTCTCTTAAAATTAGCAGGAAGATGGCCAGTAAAA |
| | | ACAATACATACAGACAATGGCAGCAATTTCACCAGTACTACGGTTAAGGCCGC |
| | | CTGTTGGTGGGCAGGGATCAAGCAGAAATTTGGCATTCCCTACAATCCCCAAA |
| | | GTAAAGGAGTAGTAGAATCTATGAATAAAGAATTAAAGAAAATTATAGGACA |
| | | GGTAAGAGATCAGGCTGAACATCTTAAGACAGCAGTACAAATGGCAGTATTC |
| | | ATCCACAATTTTAAAAGAAAAGGGGGGATTGGGGGGTACAGTGCAGGGGAA |
| | | AGAATAGTAGACATAATAGCAACAGACATACAAACTAAAGAATTACAAAAAC |
| | | AAATTACAAAAATTCAAAATTTTCGGGTTTATTACAGGGACAGCAGAGATCCA |
| | | CTTTGGAAAGGACCAGCAAAGCTTCTCTGGAAAGGTGAAGGGGCAGTAGTAA |
| | | TACAAGATAATAGTGACATAAAAGTAGTGCCAAGAAGAAAAGCAAAGATCAT |
| | | TAGGGATTATGGAAAAC (SEQ ID NO: 1054) |
| gBlock | HIVIN 66I- | gaaatTAATACGACTCACTATAgggAGCAAAAGAAATAGTAGCCAGCTGTGATA |
| | 92Q-121Y- | AATGTCAGCTAAAAGGAGAAGCCATGCATGGACAAGTAGACTGTAGTCCAGG |
| | 138A-148H- | AATATGGCAACTAGATTGTATACATTTAGAAGGAAAAATTATCCTGGTAGCAG |
| | 263K | TTCATGTAGCCAGTGGATATATAGAAGCAGAAGTTATTCCAGCACAGACAGG |
| | | GCAGGAAACAGCATACTTTCTCTTAAAATTAGCAGGAAGATGGCCAGTAAAA |
| | | ACAATACATACAGACAATGGCAGCAATTACACCAGTACTACGGTTAAGGCCG |
| | | CCTGTTGGTGGGCAGGGATCAAGCAGGCATTTGGCATTCCCTACAATCCCCAA |
| | | AGTCACGGAGTAGTAGAATCTATGAATAAAGAATTAAAGAAAATTATAGGAC |
| | | AGGTAAGAGATCAGGCTGAACATCTTAAGACAGCAGTACAAATGGCAGTATT |
| | | CATCCACAATTTTAAAAGAAAAGGGGGGATTGGGGGGTACAGTGCAGGGGA |
| | | AAGAATAGTAGACATAATAGCAACAGACATACAAACTAAAGAATTACAAAAA |
| | | CAAATTACAAAAATTCAAAATTTTCGGGTTTATTACAGGGACAGCAGAGATCC |
| | | ACTTTGGAAAGGACCAGCAAAGCTTCTCTGGAAAGGTGAAGGGGCAGTAGTA |
| | | ATACAAGATAATAGTGACATAAAAGTAGTGCCAAGAAAAAAAGCAAAGATCA |
| | | TTAGGGATTATGGAAAAC (SEQ ID NO: 1055) |
| gBlock | HIVIN 66K- | gaaatTAATACGACTCACTATAgggAGCAAAAGAAATAGTAGCCAGCTGTGATA |
| | 97A-140A- | AATGTCAGCTAAAAGGAGAAGCCATGCATGGACAAGTAGACTGTAGTCCAGG |
| | 155H | AATATGGCAACTAGATTGTAAACATTTAGAAGGAAAAATTATCCTGGTAGCAG |
| | | TTCATGTAGCCAGTGGATATATAGAAGCAGAAGTTATTCCAGCAGAGACAGG |
| | | GCAGGAAGCAGCATACTTTCTCTTAAAATTAGCAGGAAGATGGCCAGTAAAA |
| | | ACAATACATACAGACAATGGCAGCAATTTCACCAGTACTACGGTTAAGGCCGC |
| | | CTGTTGGTGGGCAGGGATCAAGCAGGAATTTGCCATTCCCTACAATCCCCAAA |
| | | GTCAAGGAGTAGTAGAATCTATGCATAAAGAATTAAAGAAAATTATAGGACA |
| | | GGTAAGAGATCAGGCTGAACATCTTAAGACAGCAGTACAAATGGCAGTATTC |
| | | ATCCACAATTTTAAAAGAAAAGGGGGGATTGGGGGGTACAGTGCAGGGGAA |
| | | AGAATAGTAGACATAATAGCAACAGACATACAAACTAAAGAATTACAAAAAC |
| | | AAATTACAAAAATTCAAAATTTTCGGGTTTATTACAGGGACAGCAGAGATCCA |
| | | CTTTGGAAAGGACCAGCAAAGCTTCTCTGGAAAGGTGAAGGGGCAGTAGTAA |
| | | TACAAGATAATAGTGACATAAAAGTAGTGCCAAGAAGAAAAGCAAAGATCAT |
| | | TAGGGATTATGGAAAAC (SEQ ID NO: 1056) |
| gBlock | HIVIN 74M- | gaaatTAATACGACTCACTATAgggAGCAAAAGAAATAGTAGCCAGCTGTGATA |
| | 140S | AATGTCAGCTAAAAGGAGAAGCCATGCATGGACAAGTAGACTGTAGTCCAGG |
| | | AATATGGCAACTAGATTGTACACATTTAGAAGGAAAAATTATCATGGTAGCAG |
| | | TTCATGTAGCCAGTGGATATATAGAAGCAGAAGTTATTCCAGCAGAGACAGG |
| | | GCAGGAAACAGCATACTTTCTCTTAAAATTAGCAGGAAGATGGCCAGTAAAA |
| | | ACAATACATACAGACAATGGCAGCAATTTCACCAGTACTACGGTTAAGGCCGC |
| | | CTGTTGGTGGGCAGGGATCAAGCAGGAATTTAGCATTCCCTACAATCCCCAAA |
| | | GTCAAGGAGTAGTAGAATCTATGAATAAAGAATTAAAGAAAATTATAGGACA |
| | | GGTAAGAGATCAGGCTGAACATCTTAAGACAGCAGTACAAATGGCAGTATTC |
| | | ATCCACAATTTTAAAAGAAAAGGGGGGATTGGGGGGTACAGTGCAGGGGAA |
| | | AGAATAGTAGACATAATAGCAACAGACATACAAACTAAAGAATTACAAAAAC |
| | | AAATTACAAAAATTCAAAATTTTCGGGTTTATTACAGGGACAGCAGAGATCCA |
| | | CTTTGGAAAGGACCAGCAAAGCTTCTCTGGAAAGGTGAAGGGGCAGTAGTAA |
| | | TACAAGATAATAGTGACATAAAAGTAGTGCCAAGAAGAAAAGCAAAGATCAT |
| | | TAGGGATTATGGAAAAC (SEQ ID NO: 1057) |
| gBlock | HIVIN 143C | gaaatTAATACGACTCACTATAgggAGCAAAAGAAATAGTAGCCAGCTGTGATA |
| | | AATGTCAGCTAAAAGGAGAAGCCATGCATGGACAAGTAGACTGTAGTCCAGG |
| | | AATATGGCAACTAGATTGTACACATTTAGAAGGAAAAATTATCCTGGTAGCAG |
| | | TTCATGTAGCCAGTGGATATATAGAAGCAGAAGTTATTCCAGCAGAGACAGG |
| | | GCAGGAAACAGCATACTTTCTCTTAAAATTAGCAGGAAGATGGCCAGTAAAA |
| | | ACAATACATACAGACAATGGCAGCAATTTCACCAGTACTACGGTTAAGGCCGC |
| | | CTGTTGGTGGGCAGGGATCAAGCAGGAATTTGGCATTCCCTGCAATCCCCAA |
| | | AGTCAAGGAGTAGTAGAATCTATGAATAAAGAATTAAAGAAAATTATAGGAC |
| | | AGGTAAGAGATCAGGCTGAACATCTTAAGACAGCAGTACAAATGGCAGTATT |
| | | CATCCACAATTTTAAAAGAAAAGGGGGGATTGGGGGGTACAGTGCAGGGGA |
| | | AAGAATAGTAGACATAATAGCAACAGACATACAAACTAAAGAATTACAAAAA |
| | | CAAATTACAAAAATTCAAAATTTTCGGGTTTATTACAGGGACAGCAGAGATCC |
| | | ACTTTGGAAAGGACCAGCAAAGCTTCTCTGGAAAGGTGAAGGGGCAGTAGTA |
| | | ATACAAGATAATAGTGACATAAAAGTAGTGCCAAGAAGAAAAGCAAAGATCA |
| | | TTAGGGATTATGGAAAAC (SEQ ID NO: 1058) |
| gBlock | HIVIN 143H | gaaatTAATACGACTCACTATAgggAGCAAAAGAAATAGTAGCCAGCTGTGATA |
| | | AATGTCAGCTAAAAGGAGAAGCCATGCATGGACAAGTAGACTGTAGTCCAGG |
| | | AATATGGCAACTAGATTGTACACATTTAGAAGGAAAAATTATCCTGGTAGCAG |
| | | TTCATGTAGCCAGTGGATATATAGAAGCAGAAGTTATTCCAGCAGAGACAGG |
| | | GCAGGAAACAGCATACTTTCTCTTAAAATTAGCAGGAAGATGGCCAGTAAAA |
| | | ACAATACATACAGACAATGGCAGCAATTTCACCAGTACTACGGTTAAGGCCGC |
| | | CTGTTGGTGGGCAGGGATCAAGCAGGAATTTGGCATTCCCCACAATCCCCAA |
| | | AGTCAAGGAGTAGTAGAATCTATGAATAAAGAATTAAAGAAAATTATAGGAC |
| | | AGGTAAGAGATCAGGCTGAACATCTTAAGACAGCAGTACAAATGGCAGTATT |
| | | CATCCACAATTTTAAAAGAAAAGGGGGGATTGGGGGGTACAGTGCAGGGGA |
| | | AAGAATAGTAGACATAATAGCAACAGACATACAAACTAAAGAATTACAAAAA |
| | | CAAATTACAAAAATTCAAAATTTTCGGGTTTATTACAGGGACAGCAGAGATCC |
| | | ACTTTGGAAAGGACCAGCAAAGCTTCTCTGGAAAGGTGAAGGGGCAGTAGTA |
| | | ATACAAGATAATAGTGACATAAAAGTAGTGCCAAGAAGAAAAGCAAAGATCA |
| | | TTAGGGATTATGGAAAAC (SEQ ID NO: 1059) |
| gBlock | HIVIN 143R | gaaatTAATACGACTCACTATAgggAGCAAAAGAAATAGTAGCCAGCTGTGATA |
| | | AATGTCAGCTAAAAGGAGAAGCCATGCATGGACAAGTAGACTGTAGTCCAGG |
| | | AATATGGCAACTAGATTGTACACATTTAGAAGGAAAAATTATCCTGGTAGCAG |
| | | TTCATGTAGCCAGTGGATATATAGAAGCAGAAGTTATTCCAGCAGAGACAGG |
| | | GCAGGAAACAGCATACTTTCTCTTAAAATTAGCAGGAAGATGGCCAGTAAAA |
| | | ACAATACATACAGACAATGGCAGCAATTTCACCAGTACTACGGTTAAGGCCGC |
| | | CTGTTGGTGGGCAGGGATCAAGCAGGAATTTGGCATTCCCCGCAATCCCCAA |
| | | AGTCAAGGAGTAGTAGAATCTATGAATAAAGAATTAAAGAAAATTATAGGAC |
| | | AGGTAAGAGATCAGGCTGAACATCTTAAGACAGCAGTACAAATGGCAGTATT |
| | | CATCCACAATTTTAAAAGAAAAGGGGGGATTGGGGGGTACAGTGCAGGGGA |
| | | AAGAATAGTAGACATAATAGCAACAGACATACAAACTAAAGAATTACAAAAA |
| | | CAAATTACAAAAATTCAAAATTTTCGGGTTTATTACAGGGACAGCAGAGATCC |
| | | ACTTTGGAAAGGACCAGCAAAGCTTCTCTGGAAAGGTGAAGGGGCAGTAGTA |
| | | ATACAAGATAATAGTGACATAAAAGTAGTGCCAAGAAGAAAAGCAAAGATCA |
| | | TTAGGGATTATGGAAAAC (SEQ ID NO: 1060) |
| gBlock | HIVIN 147G | gaaatTAATACGACTCACTATAgggAGCAAAAGAAATAGTAGCCAGCTGTGATA |
| | | AATGTCAGCTAAAAGGAGAAGCCATGCATGGACAAGTAGACTGTAGTCCAGG |
| | | AATATGGCAACTAGATTGTACACATTTAGAAGGAAAAATTATCCTGGTAGCAG |
| | | TTCATGTAGCCAGTGGATATATAGAAGCAGAAGTTATTCCAGCAGAGACAGG |
| | | GCAGGAAACAGCATACTTTCTCTTAAAATTAGCAGGAAGATGGCCAGTAAAA |
| | | ACAATACATACAGACAATGGCAGCAATTTCACCAGTACTACGGTTAAGGCCGC |
| | | CTGTTGGTGGGCAGGGATCAAGCAGGAATTTGGCATTCCCTACAATCCCCAA |
| | | GGTCAAGGAGTAGTAGAATCTATGAATAAAGAATTAAAGAAAATTATAGGAC |
| | | AGGTAAGAGATCAGGCTGAACATCTTAAGACAGCAGTACAAATGGCAGTATT |
| | | CATCCACAATTTTAAAAGAAAAGGGGGGATTGGGGGGTACAGTGCAGGGGA |
| | | AAGAATAGTAGACATAATAGCAACAGACATACAAACTAAAGAATTACAAAAA |
| | | CAAATTACAAAAATTCAAAATTTTCGGGTTTATTACAGGGACAGCAGAGATCC |
| | | ACTTTGGAAAGGACCAGCAAAGCTTCTCTGGAAAGGTGAAGGGGCAGTAGTA |
| | | ATACAAGATAATAGTGACATAAAAGTAGTGCCAAGAAGAAAAGCAAAGATCA |
| | | TTAGGGATTATGGAAAAC (SEQ ID NO: 1061) |
| gBlock | HIVIN 148H | gaaatTAATACGACTCACTATAgggAGCAAAAGAAATAGTAGCCAGCTGTGATA |
| | | AATGTCAGCTAAAAGGAGAAGCCATGCATGGACAAGTAGACTGTAGTCCAGG |
| | | AATATGGCAACTAGATTGTACACATTTAGAAGGAAAAATTATCCTGGTAGCAG |
| | | TTCATGTAGCCAGTGGATATATAGAAGCAGAAGTTATTCCAGCAGAGACAGG |
| | | GCAGGAAACAGCATACTTTCTCTTAAAATTAGCAGGAAGATGGCCAGTAAAA |
| | | ACAATACATACAGACAATGGCAGCAATTTCACCAGTACTACGGTTAAGGCCGC |
| | | CTGTTGGTGGGCAGGGATCAAGCAGGAATTTGGCATTCCCTACAATCCCCAA |
| | | AGTCACGGAGTAGTAGAATCTATGAATAAAGAATTAAAGAAAATTATAGGAC |
| | | AGGTAAGAGATCAGGCTGAACATCTTAAGACAGCAGTACAAATGGCAGTATT |
| | | CATCCACAATTTTAAAAGAAAAGGGGGGATTGGGGGGTACAGTGCAGGGGA |
| | | AAGAATAGTAGACATAATAGCAACAGACATACAAACTAAAGAATTACAAAAA |
| | | CAAATTACAAAAATTCAAAATTTTCGGGTTTATTACAGGGACAGCAGAGATCC |
| | | ACTTTGGAAAGGACCAGCAAAGCTTCTCTGGAAAGGTGAAGGGGCAGTAGTA |
| | | ATACAAGATAATAGTGACATAAAAGTAGTGCCAAGAAGAAAAGCAAAGATCA |
| | | TTAGGGATTATGGAAAAC (SEQ ID NO: 1062) |
| gBlock | HIVIN 148R | gaaatTAATACGACTCACTATAgggAGCAAAAGAAATAGTAGCCAGCTGTGATA |
| | | AATGTCAGCTAAAAGGAGAAGCCATGCATGGACAAGTAGACTGTAGTCCAGG |
| | | AATATGGCAACTAGATTGTACACATTTAGAAGGAAAAATTATCCTGGTAGCAG |
| | | TTCATGTAGCCAGTGGATATATAGAAGCAGAAGTTATTCCAGCAGAGACAGG |
| | | GCAGGAAACAGCATACTTTCTCTTAAAATTAGCAGGAAGATGGCCAGTAAAA |
| | | ACAATACATACAGACAATGGCAGCAATTTCACCAGTACTACGGTTAAGGCCGC |
| | | CTGTTGGTGGGCAGGGATCAAGCAGGAATTTGGCATTCCCTACAATCCCCAA |
| | | AGTCGAGGAGTAGTAGAATCTATGAATAAAGAATTAAAGAAAATTATAGGAC |
| | | AGGTAAGAGATCAGGCTGAACATCTTAAGACAGCAGTACAAATGGCAGTATT |
| | | CATCCACAATTTTAAAAGAAAAGGGGGGATTGGGGGGTACAGTGCAGGGGA |
| | | AAGAATAGTAGACATAATAGCAACAGACATACAAACTAAAGAATTACAAAAA |
| | | CAAATTACAAAAATTCAAAATTTTCGGGTTTATTACAGGGACAGCAGAGATCC |
| | | ACTTTGGAAAGGACCAGCAAAGCTTCTCTGGAAAGGTGAAGGGGCAGTAGTA |
| | | ATACAAGATAATAGTGACATAAAAGTAGTGCCAAGAAGAAAAGCAAAGATCA |
| | | TTAGGGATTATGGAAAAC (SEQ ID NO: 1063) |

### Hardware Development and Construction

### Microwell Array Chip Design and Fabrication

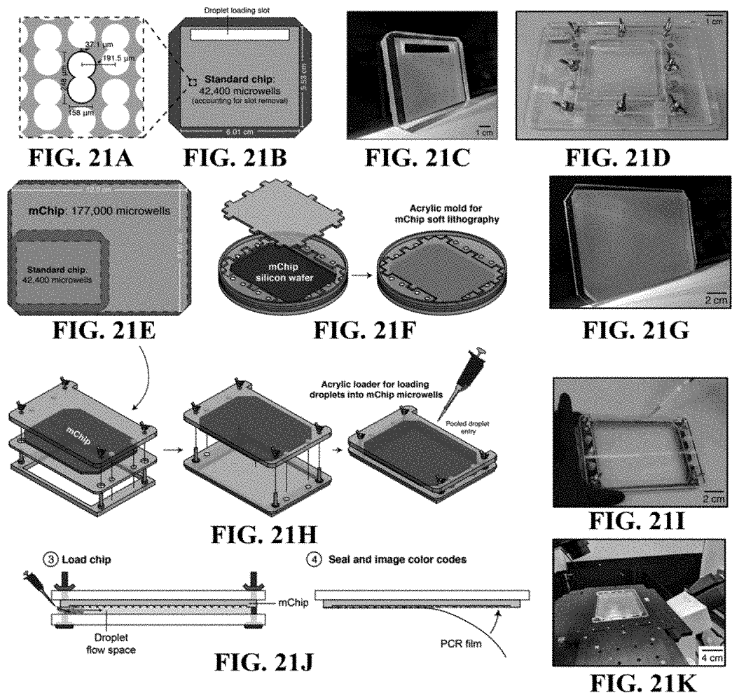

Microwell array design: Microwell dimensions were optimized by empirical testing to balance droplet loading speed (faster with larger wells) and droplet-droplet closeness inside a microwell (better merging with smaller wells). For droplets made from PCR amplification reactions or Cas13 detection mix, the optimal well geometry was achieved by joining two circles with diameters of 158 μm and an overlap of 10% (FIG. 21A). A minimum distance of 37 μm between each well facilitated consistent chip fabrication without PDMS tearing (see Microwell chip fabrication, below). Standard chips have a total microwell array that is 6.0×5.5 cm (51,496 microwells); the loading slot partially obscured the microwell array, reducing the functional array size to 6.0×˜4.5 cm (˜42,400 microwells) (FIG. 21). mChips have a microwell array that is 12×9.1 cm, bearing 177,840 microwells (FIG. 25A). The mChip microwell array is surrounded by a 0.1-0.3 cm border of PDMS to facilitate a robust seal around the edge of the chip. The total mChip dimensions were designed to maximize the number of wells that can be imaged on the area of a standard microscope stage (16×11 cm opening, Bio Precision LM Motorized Stage, Ludl Electronics), while still allowing the chip to be fabricated using standard silicon wafers (15 cm) (FIG. 25B).

Microwell chip fabrication: Polydimethylsiloxane (PDMS) chips were fabricated according to standard hard and soft lithography practices using acrylic molds to achieve consistent chip dimensions; the fabrication of standard size chips has been described previously (PNAS #1). For mChips, 150 mm wafers (WaferNet, Inc., #S64801) were washed on a spin coater (Model WS-650MZ-23NPP, Laurell Technologies) at 2500 rpm, once with acetone and once with isopropanol. Photoresist (SU-8 2050, MicroChem) was spin-coated onto each wafer in a two-step process: (1) 30 seconds, 500 rpm, acceleration 30; (2) 59 seconds, 1285 rpm, acceleration 50. Wafers were baked at 65° C. for 5 minutes and, subsequently, at 95° C. for 18 minutes. After a 1 minute cooling period, the coated wafer was placed under the appropriate photomask and irradiated (5×3 seconds, 350 W, Model 200, OAI). The wafer was baked again at 65° C. for 3 minutes and 95° C. for 9 minutes. After 1 minute of cooling, the wafer was incubated for 5 minutes under SU-8 developer. The developer was removed by spinning at 2500 rpm, and acetone and isopropanol washes were applied directly to the spinning wafer to remove excess developer and photoresist. Each wafer was characterized by visual inspection under a light microscope and profilometry to measure feature dimensions (Contour GT, Bruker). Wafers were placed inside acrylic molds and secured with magnets (FIG. 25B). To fabricate chips from the molds, PDMS was mixed and poured into the mold, and the entire mold was placed under vacuum for 3-5 min. The mold was closed with an acrylic lid to achieve uniform chip thickness, and the chips were baked for at least 2 hours. After the chip was removed from the mold, the surface of the chip bearing the microwell array and the sides (but not the back of the chip opposite the microwell array) were coated with 1.5 μm Parylene C (Paratronix/MicroChem, Westborough, Mass.). Chips were stored in plastic bags at room temperature until use.

Acrylic device fabrication (molds and loaders): Molds (PNAS #1) and loaders (PNAS #2) for standard chip production and handling were constructed as described previously. Similar methods were used to construct molds and loaders for mChip (FIG. 25B). Briefly, 12″×12″ cast acrylic sheets (¼″ or ⅛″, clear or black) were purchased from Amazon (Small Parts, #B004N1JLI4). Mold and loader designs were created in AutoCAD (AutoDesk), and parts were cut using an Epilog Fusion M2 laser cutter (60 W). Acrylic parts were fused together by wetting with dichloromethane (Sigma Aldrich). N42 Neodymium disc magnets (Applied Magnets, Inc., Plano, Tex.) were added to devices with epoxy (Loctite, Metal/Concrete). Cap screws (M4×25), nuts (M4), and washers (M4) were purchased from Thorlabs.

### Color Code Design, Construction, and Characterization

Color code design: Color codes served as optical unique solution identifiers for each reagent (e.g. detection mix or amplified sample) that was emulsified into droplets. The original 64 color code set was made from ratios of 3 fluorescent dyes, such that the total concentration of the three dyes ([dye 1]+[dye 2]+[dye 3]) was constant and served as an internal control to normalize for variation in illumination across the field of view or at different locations on the chip (PNAS #1). The working total dye concentration for the 64 color code set was 1-5 μM, as described previously (PNAS #1). The 1050 color codes were designed by (1) increasing the total working concentration of the 3 fluorescent dyes to 20 μM, such that 210 color codes could be faithfully identified in 3-color space (FIG. 24A and FIG. 24B), and (2) adding a fourth fluorescent dye at one of five concentrations (0, 3, 7, 12 or 20 μM) to multiply the 210 codes by five (FIG. 24A). In this design, each of the 4 dye intensities is normalized to the sum of the first 3 fluorescent dyes.

Color code construction: The standard 64 color code set (50 μM stock concentration; 1-5 μM working concentration) was constructed as previously described (PNAS #1). The 210 color codes (400 μM stock concentration; 20 μM working concentration) were constructed using similar methods, as follows. Alexa Fluor 647 (AF647), Alexa Fluor 594 (AF594), Alexa Fluor 555 (AF555), and Alexa Fluor 405 NHS ester (AF405-NHS) (Thermo Fisher) were diluted to 25 mM in DMSO (Sigma). Since the molar masses of these dyes is proprietary, the following approximate masses provided by the manufacturer were used for calculations: AF647: 1135 g/mol; AF594: 1026 g/mol; AF555: 1135 g/mol; AF405-NHS: 1028 g/mol. Dye stocks in DMSO were further diluted to 400 μm in DNase/RNase-free water (Life Technologies). Alexa Fluor 405 NHS ester was incubated at room temperature for one hour to allow hydrolysis of the NHS ester and generate Alexa Fluor 405 (AF405). Custom Matlab scripts were used to calculate the dye volumes to combine to evenly distribute 210 color codes across 3-color space (Table 10b). 3-color dye combinations (made from AF647, AF594, and AF555) were constructed in 96 well plates (Eppendorf) using a Janus Mini liquid handler (Perkin Elmer). To construct 1050 color codes, AF405 was manually diluted to five concentrations (0, 60, 140, 240, and 400 μm), and each concentration was arrayed across a 96 well plate. Each of the 210 color codes (10 μL) and AF405 (10 μL) were combined and mixed in a fresh 96 well plate using a Bravo (supplier). The final stock concentration of the sum of AF647, AF594 and AF555 was 200 μM; the final concentrations of AF405 were 0, 30, 70, 120, and 200 μM. Stocks were diluted 1:10 into amplified samples or detection mixes for use.

Characterization of 1050 color code set: Each color code was diluted 1:10 in LB broth (a medium that yields droplets of similar size to droplets made from PCR products and detection reagents) to a final total 3-dye concentration of 20 μM. Each solution was emulsified into droplets as described in Section II.D., above. The fidelity of the color code strategy was measured as described previously [PNAS #1].

Table 10a-10b In Tables 10a and 10b, each row represents a color code. Each column gives the volume (μm) of one of the three dyes. The total volume for each code is 50 μL.

TABLE 10a
64 Color Codes.

| Alexa Fluor | Alexa Fluor | Alexa Fluor |
|---|---|---|
| 555 volume | 594 volume | 647 volume |
| 0 | 50 | 0 |
| 3 | 29 | 18 |
| 7 | 17 | 27 |
| 10 | 8 | 31 |
| 15 | 4 | 31 |
| 19 | 4 | 27 |
| 24 | 8 | 17 |
| 29 | 17 | 4 |
| 1 | 0 | 49 |
| 3 | 33 | 13 |
| 7 | 21 | 22 |
| 11 | 13 | 27 |
| 15 | 8 | 27 |
| 20 | 8 | 22 |
| 25 | 13 | 13 |
| 33 | 0 | 17 |
| 1 | 4 | 45 |
| 4 | 38 | 9 |
| 7 | 25 | 18 |
| 11 | 17 | 22 |
| 15 | 13 | 22 |
| 20 | 13 | 18 |
| 25 | 17 | 9 |
| 33 | 4 | 13 |
| 1 | 8 | 40 |
| 4 | 42 | 4 |
| 8 | 29 | 13 |
| 11 | 21 | 18 |
| 16 | 17 | 18 |
| 20 | 17 | 13 |
| 25 | 21 | 4 |
| 33 | 8 | 8 |
| 2 | 13 | 36 |
| 5 | 0 | 45 |
| 8 | 33 | 9 |
| 12 | 25 | 13 |
| 16 | 21 | 13 |
| 21 | 21 | 9 |
| 28 | 0 | 22 |
| 34 | 13 | 4 |
| 2 | 17 | 31 |
| 6 | 4 | 40 |
| 8 | 38 | 4 |
| 12 | 29 | 9 |
| 16 | 25 | 9 |
| 21 | 25 | 4 |
| 28 | 4 | 17 |
| 37 | 0 | 13 |
| 2 | 21 | 27 |
| 6 | 8 | 36 |
| 10 | 0 | 40 |
| 12 | 33 | 4 |
| 17 | 29 | 4 |
| 24 | 0 | 26 |
| 29 | 8 | 13 |
| 38 | 4 | 8 |
| 3 | 25 | 22 |
| 6 | 13 | 31 |
| 10 | 4 | 36 |
| 14 | 0 | 36 |
| 19 | 0 | 31 |
| 24 | 4 | 22 |
| 29 | 13 | 8 |
| 38 | 8 | 4 |

TABLE 10b
210 Color Codes

| Alexa Fluor | Alexa Fluor | Alexa Fluor |
|---|---|---|
| 555 volume | 594 volume | 647 volume |
| 0 | 0 | 50 |
| 0 | 3 | 47 |
| 0 | 5 | 45 |
| 0 | 8 | 42 |
| 0 | 11 | 39 |
| 0 | 13 | 37 |
| 0 | 16 | 34 |
| 0 | 18 | 32 |
| 0 | 21 | 29 |
| 0 | 24 | 26 |
| 0 | 26 | 24 |
| 0 | 29 | 21 |
| 0 | 32 | 18 |
| 0 | 34 | 16 |
| 0 | 37 | 13 |
| 0 | 39 | 11 |
| 0 | 42 | 8 |
| 0 | 45 | 5 |
| 0 | 47 | 3 |
| 0 | 50 | 0 |
| 3 | 0 | 47 |
| 3 | 3 | 45 |
| 3 | 5 | 42 |
| 3 | 8 | 39 |
| 3 | 11 | 37 |
| 3 | 13 | 34 |
| 3 | 16 | 32 |
| 3 | 18 | 29 |
| 3 | 21 | 26 |
| 3 | 24 | 24 |
| 3 | 26 | 21 |
| 3 | 29 | 18 |
| 3 | 32 | 16 |
| 3 | 34 | 13 |
| 3 | 37 | 11 |
| 3 | 39 | 8 |
| 3 | 42 | 5 |
| 3 | 45 | 3 |
| 3 | 47 | 0 |
| 5 | 0 | 45 |
| 5 | 3 | 42 |
| 5 | 5 | 39 |
| 5 | 8 | 37 |
| 5 | 11 | 34 |
| 5 | 13 | 32 |
| 5 | 16 | 29 |
| 5 | 18 | 26 |
| 5 | 21 | 24 |
| 5 | 24 | 21 |
| 5 | 26 | 18 |
| 5 | 29 | 16 |
| 5 | 32 | 13 |
| 5 | 34 | 11 |
| 5 | 37 | 8 |
| 5 | 39 | 5 |
| 5 | 42 | 3 |
| 5 | 45 | 0 |
| 8 | 0 | 42 |
| 8 | 3 | 39 |
| 8 | 5 | 37 |
| 8 | 8 | 34 |
| 8 | 11 | 32 |
| 8 | 13 | 29 |
| 8 | 16 | 26 |
| 8 | 18 | 24 |
| 8 | 21 | 21 |
| 8 | 24 | 18 |
| 8 | 26 | 16 |
| 8 | 29 | 13 |
| 8 | 32 | 11 |
| 8 | 34 | 8 |
| 8 | 37 | 5 |
| 8 | 39 | 3 |
| 8 | 42 | 0 |
| 11 | 0 | 39 |
| 11 | 3 | 37 |
| 11 | 5 | 34 |
| 11 | 8 | 32 |
| 11 | 11 | 29 |
| 11 | 13 | 26 |
| 11 | 16 | 24 |
| 11 | 18 | 21 |
| 11 | 21 | 18 |
| 11 | 24 | 16 |
| 11 | 26 | 13 |
| 11 | 29 | 11 |
| 11 | 32 | 8 |
| 11 | 34 | 5 |
| 11 | 37 | 3 |
| 11 | 39 | 0 |
| 13 | 0 | 37 |
| 13 | 3 | 34 |
| 13 | 5 | 32 |
| 13 | 8 | 29 |
| 13 | 11 | 26 |
| 13 | 13 | 24 |
| 13 | 16 | 21 |
| 13 | 18 | 18 |
| 13 | 21 | 16 |
| 13 | 24 | 13 |
| 13 | 26 | 11 |
| 13 | 29 | 8 |
| 13 | 32 | 5 |
| 13 | 34 | 3 |
| 13 | 37 | 0 |
| 16 | 0 | 34 |
| 16 | 3 | 32 |
| 16 | 5 | 29 |
| 16 | 8 | 26 |
| 16 | 11 | 24 |
| 16 | 13 | 21 |
| 16 | 16 | 18 |
| 16 | 18 | 16 |
| 16 | 21 | 13 |
| 16 | 24 | 11 |
| 16 | 26 | 8 |
| 16 | 29 | 5 |
| 16 | 32 | 3 |
| 16 | 34 | 0 |
| 18 | 0 | 32 |
| 18 | 3 | 29 |
| 18 | 5 | 26 |
| 18 | 8 | 24 |
| 18 | 11 | 21 |
| 18 | 13 | 18 |
| 18 | 16 | 16 |
| 18 | 18 | 13 |
| 18 | 21 | 11 |
| 18 | 24 | 8 |
| 18 | 26 | 5 |
| 18 | 29 | 3 |
| 18 | 32 | 0 |
| 21 | 0 | 29 |
| 21 | 3 | 26 |
| 21 | 5 | 24 |
| 21 | 8 | 21 |
| 21 | 11 | 18 |
| 21 | 13 | 16 |
| 21 | 16 | 13 |
| 21 | 18 | 11 |
| 21 | 21 | 8 |
| 21 | 24 | 5 |
| 21 | 26 | 3 |
| 21 | 29 | 0 |
| 24 | 0 | 26 |
| 24 | 3 | 24 |
| 24 | 5 | 21 |
| 24 | 8 | 18 |
| 24 | 11 | 16 |
| 24 | 13 | 13 |
| 24 | 16 | 11 |
| 24 | 18 | 8 |
| 24 | 21 | 5 |
| 24 | 24 | 3 |
| 24 | 26 | 0 |
| 26 | 0 | 24 |
| 26 | 3 | 21 |
| 26 | 5 | 18 |
| 26 | 8 | 16 |
| 26 | 11 | 13 |
| 26 | 13 | 11 |
| 26 | 16 | 8 |
| 26 | 18 | 5 |
| 26 | 21 | 3 |
| 26 | 24 | 0 |
| 29 | 0 | 21 |
| 29 | 3 | 18 |
| 29 | 5 | 16 |
| 29 | 8 | 13 |
| 29 | 11 | 11 |
| 29 | 13 | 8 |
| 29 | 16 | 5 |
| 29 | 18 | 3 |
| 29 | 21 | 0 |
| 32 | 0 | 18 |
| 32 | 3 | 16 |
| 32 | 5 | 13 |
| 32 | 8 | 11 |
| 32 | 11 | 8 |
| 32 | 13 | 5 |
| 32 | 16 | 3 |
| 32 | 18 | 0 |
| 34 | 0 | 16 |
| 34 | 3 | 13 |
| 34 | 5 | 11 |
| 34 | 8 | 8 |
| 34 | 11 | 5 |
| 34 | 13 | 3 |
| 34 | 16 | 0 |
| 37 | 0 | 13 |
| 37 | 3 | 11 |
| 37 | 5 | 8 |
| 37 | 8 | 5 |
| 37 | 11 | 3 |
| 37 | 13 | 0 |
| 39 | 0 | 11 |
| 39 | 3 | 8 |
| 39 | 5 | 5 |
| 39 | 8 | 3 |
| 39 | 11 | 0 |
| 42 | 0 | 8 |
| 42 | 3 | 5 |
| 42 | 5 | 3 |
| 42 | 8 | 0 |
| 45 | 0 | 5 |
| 45 | 3 | 3 |
| 45 | 5 | 0 |
| 47 | 0 | 3 |
| 47 | 3 | 0 |
| 50 | 0 | 0 |

Characterization in 3-color space: The fidelity of the color code strategy in 3-color space was measured as described previously⁸. Each color code in 3-color space was assigned to one of three chips. Assignments were made to maximize the separation between the color codes on any chip, and each chip received 1/3 of the color codes (70 total) (FIGS. 38B and 38C). Droplets from color codes assigned to Chip 1 (70 3-color codes×5 UV concentrations=350 droplet emulsions) were pooled and loaded onto a standard chip. Chips 2 and 3 were prepared in a similar manner. The chips were imaged (note that no merging was performed in color code characterization experiments), and each droplet was computationally assigned to a color code cluster. The experimental results from Chips 1, 2, and 3 served as “ground truth” assignments. The data from Chips 1, 2, and 3 were then computationally combined, effectively increasing the density of color code clusters in 3-color space, and the droplets were reassigned to color code clusters in this more crowded 3-color space (FIGS. 38B and 38C). Finally, a sliding distance filter was applied to remove droplets at the edges of clusters or in between clusters, and the droplets were reassigned to color code clusters (FIGS. 38B and 38F). The sliding distance filter refers to a radius around each cluster centroid that is used to remove droplets that fall in the space between clusters (FIG. 38F). The radius may be larger (to include more droplets) or smaller (to more stringently filter out droplets). New assignments were compared to “ground truth” assignments to measure the percent of droplets that would be misclassified if the color codes were not separated over three chips (FIGS. 38C and 38D). In the work presented here, the radius of the sliding distance filter was set to achieve at least 99.5% correct classification in the test data set, corresponding to the removal of 6% of droplets.

Characterization along the 4th-color dimension: The five concentrations of the 4th fluorescent dye were divided between two chips (Chip 1: 0, 7, 20 μM; Chip 2: 3, 12 μM) (FIG. 38E). Droplets from dye intensities assigned to Chip 1 (3 UV intensities×210 color codes=620 emulsions) were pooled and loaded onto a standard chip. Chip 2 was prepared in a similar manner but with fewer pooled emulsions (2 UV intensities×210 color codes=420 emulsions). The chips were imaged (note that no merging was performed in color code characterization experiments), and each droplet was computationally assigned to a UV intensity bin. The experimental results from Chips 1 and 2 served as “ground truth” assignments. The data from Chips 1 and 2 were then computationally combined, effectively increasing the density of UV intensity bins along the 4th-color dimension, and the droplets were reassigned to UV intensity bins in this more crowded space (FIG. 38E). Finally, a sliding distance filter was applied to remove droplets at the edges of intensity bins or in between intensity bins, and the droplets were reassigned to UV intensity bins (FIG. 38E). New assignments were compared to “ground truth” assignments to measure the percent of droplets that would be misclassified if the UV intensities were not separated over three chips (FIG. 38E). As classification in the 4^{th}-color dimension is sufficiently high (>99.5% accurate) without filtering, no filtering in the 4^{th}-color dimension was applied to the experimental data.

Microwell array statistics: The number of tests that can be performed on one chip depends on the number of productive droplet pairs per chip and the number of replicates per test that are required to make an accurate call.

First, factors affecting the number of productive droplet pairs per chip are considered: The microwell array of a standard chip contains ˜42,000 microwells. By empirical observation, loading efficiency is ˜70%, and an additional ˜10% of microwells are lost to color code filtering (see below). Finally, stochastic droplet pairing produces ˜50% productive droplet pairs (one droplet containing amplified sample and one droplet containing detection mix). Overall, ˜10,000-14,000 droplet pairs produce useful data per chip. The mChip microwell array contains ˜177,000 microwells, resulting in ˜65,000 useful droplet pairs/chip.

Second, factors affecting the number of replicates per test required to make an accurate call chip are considered: The vast majority of positive detection reactions have high signal above background and little replicate-to-replicate variability, and color code classification is very good (>99.5% accuracy after filtering, see FIG. 38A-38G), suggesting that the number of requisite replicates per test could be quite low. As an experimental measure of the number of replicates needed to correctly identify signal above background, bootstrap analysis was performed on CARMEN-Cas13 Zika detection data (FIG. 22A-22E and Materials and Methods), revealing a minimum of 3 replicates to correctly call signal above background in >99.9% of bootstrap samples.

It should be noted that the number of replicates required to make an accurate call varies by application type. For nucleic acid detection, which is a near-binary readout, 3 replicates is sufficient. However, for SNP discrimination, which relies on differentiating the relative reaction rates of two crRNAs with a given target, bootstrap analysis suggests that 10-15 replicates are necessary (data not shown). Additionally, for quantitative applications, many replicates may be necessary to yield a result within a desired tolerance (e.g. 5%) of the ground truth value.

Finally, how to calculate the number of tests that can be performed on one chip are discussed using the values determined above. Droplet pairing in the microwell array is stochastic; thus, the distribution of the number of replicates per test is Poisson. The user can set the average number of replicates per test (the average of the Poisson distribution) higher or lower to control the probability of test dropout due to undersampling. For example, using an average of 12 replicates per test, the probability of any test being uninterpretable because of a lack of replicates (<3 replicates) is 1 in 2,000. For a standard chip (˜12,000 productive droplet pairs), an average of 12 replicates per test permits 1,000 tests per chip with a dropout rate well below 1 per chip (1 in 2000). For mChip, which yields ˜65,000 droplet pairs, performing 5,000 tests per chip results in an average of 14 replicates per test and reduces the probability of dropout to 1 in 10,000 (below 1 per chip). In situations where delivering a result for every test is essential, such as clinical diagnostics, the average replicate level can be further increased to ensure that sampling for every test is high and the dropout rate due to undersampling is vanishingly low.

Controlling exchange of solutes between droplets during pooling: The kinetics of small molecule exchange in the droplet-microwell platform have been previously described⁸. Small molecules may partition into surfactant micelles and exchange between droplets during the pooling step, which lasts <10 min. The exchange of fluorescent dyes during pooling is negligible and does not compromise color code classification⁸. Once droplets are loaded into the microwell array, the Parylene-coated walls of the PDMS microwells prevent further exchange⁸. Advantageously, diffusion of larger hydrophilic or charged molecules is not a concern in the system since the surfactant-dependent mechanisms by which small molecules can exit droplets are neither expected nor observed to enable protein or nucleic acid escape. Indeed, commercially available systems for ultra-sensitive nucleic acid detection based on similar oils, surfactants, and buffers (e.g. digital droplet PCR) are well-established.

Flexibility of experimental design: The number of tests on a chip is the product of the number of samples and the number of detection mixes, which can be determined by the needs of a user (e.g. 10 samples×100 detection mixes, or 100 samples×10 detection mixes). Notably, CARMEN shines in cases when the test matrix is approximately square: the number of samples and detection mixes are both high (e.g. >10). To perform such an experiment conventionally, liquid handling (whether manual or robotic) is complex and time-consuming, reagent consumption is costly (see cost analysis below), and testing may be sample-limited. CARMEN circumvents these issues using miniaturization and droplet self-organization (see main text). For use-cases where high sample throughput alone is desired (many samples×1 detection mix), CARMEN dramatically reduces costs (see below), but the experiment setup is linear (samples×1), so a multichannel pipet is equally time-efficient. For use-cases where multiplexed detection alone is desired (1 sample×many detection mixes), the user may consider metagenomic sequencing if the sensitivity is sufficient for the application, while CARMEN may be ideal in cases where exquisite sensitivity and extensive multiplexing is required.

Color code analysis: Color code classification is robust (FIG. 38A-38G). After creating and characterizing a set of color codes, the codes are used out-of-the-fridge for each experiment with no additional calibration. Normalizing each color code to the sum of the three fluorescent dyes comprising the 3-color space (Alexa Fluors 647, 594, and 555) makes the system robust to fluorescence imaging artifacts, and discrete color code clusters readily appear. Each cluster represents a droplet set with known contents (e.g. droplets from detection mix 4). Indeterminate points in color space are filtered out by introducing a threshold for the maximum distance a droplet's color code can be from the center of its color code cluster (i.e. a distance threshold, see Materials and Methods). In the rare case where one color code cluster begins to overlap another, only the two clashing clusters are impacted (and can almost always be resolved, albeit with a loss of replicates), leaving the rest of the color codes unaffected. Such clashing color codes may be omitted from future experiments without any detrimental effect on the set as a whole, and the user does not have to recreate the entire color code set.

False negatives and false positives due to color code misclassification: If enough replicates of a test are misclassified, the outcome of the test could change. The fluorescence value of a test is the median value of all replicates; for the median of a positive test to drop to background (i.e. become a false negative), the majority of the replicates would have to be misclassified droplet pairs with no signal above background (dark droplet pairs). Since the detection matrix is sparse, the odds of a misclassified droplet pair being a dark droplet pair are high (99% in the human-associated virus panel testing). This dramatically increases the odds of false negatives compared to false positives. For false negatives, assuming a droplet misclassification rate of 0.005 (see infra and FIG. 38A-38G), the probability of a droplet pair being misclassified is 0.01. With 5 replicates, the odds of the majority of replicates being misclassified is 0.01×0.01×0.01×(5 choose 3)=1 in 100,000. Increasing to 7 replicates improves the odds to <1 in 2 million. Thus, in situations where ensuring accurate calls is critical, such as clinical diagnostics, the number of replicates may be increased to dramatically decrease the odds of a miscalled test due to droplet misclassification.

Cost and sample consumption analysis: A key advantage of CARMEN-Cas13 is that it miniaturizes Cas13 detection reactions, thereby reducing reagent and sample consumption per test. Reagent and consumables costs dominate when testing dozens of samples against hundreds of targets using conventional large-volume (10s of microliters) assays, such as SHERLOCK, DETECTR, qPCR, ELISA, and LAMP. Thus, Applicants sought to quantify the cost advantage conferred by CARMEN over these methods when testing many samples against many targets.

To analyze the costs associated with CARMEN-Cas13, Applicants first considered the cost of detection reagents alone, and then considered additional costs (plastics including arrays, droplet generation, and color codes). CARMEN-Cas13 typically reduces detection volumes by >400-fold per test, (from 92 microliters to perform 4 replicates of a standard 20 ul detection reaction to less than 0.2 microliters to perform a CARMEN-Cas13 test with an average of 10 replicate droplet pairs). This results in a >300-fold reduction in cost relative to SHERLOCK, as Applicants use a 4x higher concentration of the fluorescent cleavage reporter in CARMEN-Cas13 (see Table 11). Accounting for an additional fixed cost per chip and the cost of color coding and emulsifying samples, the cost per test for CARMEN-Cas13 is >100-fold cheaper than the equivalent SHERLOCK test (see Table 11).

TABLE 11
Consumables cost calculation concerning CARMEN-Cas13.

| Category | Cost (USD) | Notes |
|---|---|---|
| Fixed cost per chip | $16.00 | Includes oil, surfactant, chip |
| | | itself |
| Marginal cost per | $2.24 | Includes PCR reagents, droplet |
| sample | | generation, color codes |
| Marginal cost per | $5.34 | Includes detection reagents, |
| detection mix | | droplet generation, color codes |

| | # detection | Number of | Total CARMEN | Cost per |
|---|---|---|---|---|
| # samples | mixes | Tests | Cost (USD) | test (USD) |
| 20 | 20 | 400 | $167.57 | $0.42 |
| 100 | 50 | 5000 | $506.90 | $0.10 |
| 200 | 100 | 20,000 | $1,045.80 | $0.05 |

| SHERLOCK in a plate | | | | |
|---|---|---|---|---|
| | Detection | | Volume per | Cost per |
| Cost per ul | volume (ul) | Replicates | test (ul) | test |
| 0.06 | 20 | 4 | 92 | $5.52 |

Equipment costs for CARMEN are high, but are not dramatically higher than other multiplexed methods for nucleic acid detection and could be improved in the future. Like many other methods using a fluorescent readout (qPCR, FISH), CARMEN-Cas13 requires sensitive detection of fluorescence in 4-5 channels. CARMEN-Cas13 also requires some automated imaging capabilities to facilitate data acquisition from the microwell array. Multimode plate readers or qPCR machines cost about $30,000, whereas a microscope suitable for CARMEN costs about $50,000 (the additional cost coming from the imaging requirements for CARMEN). Both of these are much cheaper than Illumina sequencing machines typically used for high-throughput metagenomic sequencing (e.g. HiSeq, NextSeq, NovaSeq).

In addition to equipment for fluorescent readout, CARMEN also requires equipment for droplet generation. While a commercial machine, the Bio-Rad QX200 ($31,000), can be used for droplet generation, the equipment requirements for droplet generation can be substantially reduced by using a custom-fabricated pressure manifold, which costs approximately $2,000 to make. Thus, droplet generation hardware is a minor component of the CARMEN technology's overall cost.

While labor costs are difficult to quantify, the amount of labor required for CARMEN-Cas13 is lower per test than for low-plex assays like RT-qPCR, ELISAs, or LAMP. Although it takes, for example, ˜8 person-hours to set up, image, and analyze an individual mChip, the ˜5,000 tests per chip is equivalent to >50 full 384-well plates (containing 3-4 technical replicates per test, the number necessary to achieve statistical power in plate-based assays). Thus, the time required per full 384-well plate equivalent is <10 person-minutes; in Applicants' hands, setting up one full 384-well plate takes at least an hour; starting with thawed reagents and ending at the start of the assay. In addition, the protocol for CARMEN-Cas13 is simpler than library preparation for next-generation sequencing, requiring fewer steps and less time to complete.

It should be noted that the scale of the experiment is important to consider when comparing the costs of performing CARMEN-Cas13 relative to other assays. In particular, many of the associated costs scale with the number of chips, or linearly with the sum of the number of amplified samples and the number of Cas13 detection mixes. As such, a less favorable use case for CARMEN-Cas13 would be testing 1 sample for hundreds of potential viruses: due to the fixed costs, the cost savings will be smaller relative to performing the same experiment in a standard microtiter plate. The cost drops substantially when multiple samples are tested simultaneously, as the marginal cost of adding a new sample to a particular chip is only a few dollars. The combinatorial nature of CARMEN further reduces the cost of testing many samples for the presence of many targets. It should be noted that in the limit of low reagent cost per test, sample processing will likely dominate total cost, as sample costs scale with the number of samples rather than the number of tests being performed. Thus, to enable sample testing at even higher throughput than CARMEN-Cas13, one would need to significantly reduce the cost and labor associated with sample collection and processing.

Finally, performing dozens or hundreds of SHERLOCK, DETECTR, qPCR, ELISA, or LAMP assays on a patient sample requires a very large sample volume (tens of milliliters of blood, saliva, or urine), which is often not available. For CARMEN, at most 2 microliters of extracted RNA are used per PCR pool, for a total of up to 30 microliters for 15 PCR pools in the human-associated viral panel. This requires a total sample input volume of a few hundred microliters of bodily fluid (depending on the type of extraction kit used). In short, the overall input sample volume requirements for CARMEN do not vary substantially from other methods, despite a considerable increase in the number of tests performed on each sample. Thus, in addition to reducing reagent costs, CARMEN-Cas13 reduces sample consumption, thereby enabling more tests to be run and reducing sample acquisition and processing costs.

### Human-Associated Viral Panel

Selection of optimal crRNAs for testing: Due to the high cost of synthesizing hundreds of synthetic DNA and RNA oligonucleotides, Applicants did not test the entirety of the human-associated viral panel design experimentally. The vast majority (143) of species required a single crRNA to cover 90% of known sequences (FIG. 39A-39G), thus A[[;ocamts decided to test a single crRNA for each species. In cases where there were multiple crRNAs in a set, the crRNA whose sequence most closely matched the majority consensus sequence for the species was chosen. Based on the results using crRNA sets for sub-subtyping of influenza A (FIG. 42A-42C), it is likely that one could use the complete crRNA sets to fully cover 90% of the known sequences in each species, as designed. Applicants' barcode and multiplexing scheme would be able to accommodate this, with a moderate decrease in sample throughput due to the increased number of detection mixes.

Cross-contamination: A practical concern of testing a massively multiplexed viral detection panel is cross-contamination, especially pre-emulsification. The extreme sensitivity of the CARMEN-Cas13 system means that even trace cross-contamination could lead to widespread false-positive results. Widespread cross-reactivity was not observed during Applicatns' testing, however there were some examples of cross-reactivity between a crRNA and an unexpected synthetic target. All examples of cross-reactivity were investigated by aligning crRNA and synthetic target sequences. Based on this analysis, a handful (4-5) of these examples were likely sequence-mediated, and were modified in the version 2 redesign. The remaining examples of cross-reactivity are likely due to cross-contamination for the following reasons:
1. The vast majority of cross-reactivity that was not sequence-mediated occurred between neighboring wells, suggesting that it could be due to cross-contamination during the dilution of synthetic targets, or during the setup of amplification reactions.
2. It is possible that the cross-reactivity is due to cross-contamination that occurred during DNA or RNA synthesis. The oligonucleotides for the human-associated virus panel were synthesized commercially, in parallel, in 96-well plates. Co-synthesized oligonucleotides used as barcoded adapters for next-generation sequencing have been observed to have cross-contamination at low frequencies”.

Sequence coverage: In addition to cross-reactivity, sequence coverage is an important aspect of design. The human-associated virus panel was designed to cover at least 90% of known sequences for each species, but the actual coverage might be higher or lower for the following reasons.
1. The crRNAs and primers were designed to cover at least 90% of the known sequences for each species in the panel, but it is possible that they could also detect the 5-10% of known sequences that are not supposed to be covered by design.
2. Applicants set a stringent threshold of 1 mismatch between a crRNA and its target. Depending on the position of the mismatch, there could still be substantial cleavage activity; truncated spacers can be quite active for nucleic acid detection⁷.
3. For some species, not have enough sequence data is available to design an accurate diagnostic; thus Applicants restricted the panel to species with ≥10 available genome sequences.

Similar considerations also apply to the influenza subtyping panel.

Finally, sequence coverage and analytical sensitivity are distinct but related considerations that contribute to assay sensitivity: a given crRNA targets a specific sequence within the genome with a certain analytical sensitivity (ability to detect that sequence above background). To increase assay sensitivity, a user may add more crRNAs to be able to detect additional fragments of pathogen nucleic acid (increasing sequence coverage) or improve the performance of individual crRNAs. Multiplexing crRNAs to increase sequence coverage is particularly effective when samples may carry only a portion of the known viral genome (due to degradation, mutation, etc.).

Testing of unknown samples: In this study, Applicants tested 169 known, synthetic targets with the majority consensus sequence of each of the 169 species in the human-associated viral panel, using a single primer pool to amplify each target (based on the design). For unknown samples, one would amplify each sample with all 15 pools, and then either combine the pools prior to detection, or run them separately. The following outcomes are possible:
1. One may observe selective identification with a single crRNA and rejoice.
2. If one observes cross-reactivity, one can rerun the individual pool where the cross-reactivity occurred. In these cases, one should not assume that there is a co-infection, unless there is prior information suggesting that a co-infection is likely.
3. Weak reactivity may be accounted for by using positive controls or retesting samples to increase the confidence in the result.
4. No positive results may be observed for the following reasons: (1) the sequence of the pathogen is in the 5-10% of known sequences not covered by the design; (2) the viral titers could be too low to detect; or (3) the sample could be degraded.

The following references are relevant to Example 2:
1. Bosch, I. et al. Rapid antigen tests for dengue virus serotypes and Zika virus in patient serum. Sci. Transl. Med. 9, (2017).
2. Popowitch, E. B., O'Neill, S. S. & Miller, M. B. Comparison of the Biofire FilmArray RP, Genmark eSensor RVP, Luminex xTAG RVPv1, and Luminex xTAG RVP fast multiplex assays for detection of respiratory viruses. J. Clin. Microbiol. 51, 1528-1533 (2013).
3. Du, Y. et al. Coupling Sensitive Nucleic Acid Amplification with Commercial Pregnancy Test Strips. Angew. Chem. Int. Ed Engl. 56, 992-996 (2017).
4. Wang, D. et al. Microarray-based detection and genotyping of viral pathogens. Proc. Natl. Acad. Sci. U.S.A 99, 15687-15692 (2002).
5. Houldcroft, C. J., Beale, M. A. & Breuer, J. Clinical and biological insights from viral genome sequencing. Nat. Rev. Microbiol. 15, 183-192 (2017).
6. Palacios, G. et al. Panmicrobial oligonucleotide array for diagnosis of infectious diseases. Emerg. Infect. Dis. 13, 73-81 (2007).
7. Gootenberg, J. S. et al. Nucleic acid detection with CRISPR-Cas13a/C2c2. Science 356, 438-442 (2017).
8. Kulesa, A., Kehe, J., Hurtado, J. E., Tawde, P. & Blainey, P. C. Combinatorial drug discovery in nanoliter droplets. Proc. Natl. Acad. Sci. U.S.A 115, 6685-6690 (2018).
9. Chertow, D. S. Next-generation diagnostics with CRISPR. Science 360, 381-382 (2018).
10. Kocak, D. D. & Gersbach, C. A. From CRISPR scissors to virus sensors. Nature 557, 168-169 (2018).
11. US Food & Drug Administration. Available at: www.fda.gov. (Accessed: 1 Nov. 2018)
12. Brister, J. R., Rodney Brister, J., Ako-adjei, D., Bao, Y. & Blinkova, O. NCBI Viral Genomes Resource. Nucleic Acids Res. 43, D571-D577 (2014).
13. Briese, T. et al. Virome Capture Sequencing Enables Sensitive Viral Diagnosis and Comprehensive Virome Analysis. MBio 6, e01491-15 (2015).
14. Allicock, O. M. et al. BacCapSeq: a Platform for Diagnosis and Characterization of Bacterial Infections. MBio 9, (2018).
15. Chen, J. S. et al. CRISPR-Cas12a target binding unleashes indiscriminate single-stranded DNase activity. Science 360, 436-439 (2018).
16. Gootenberg, J. S. et al. Multiplexed and portable nucleic acid detection platform with Cas13, Cas12a, and Csm6. Science 360, 439-444 (2018).
17. Myhrvold, C. et al. Field-deployable viral diagnostics using CRISPR-Cas13. Science 360, 444-448 (2018).
18. Macosko, E. Z. et al. Highly Parallel Genome-wide Expression Profiling of Individual Cells Using Nanoliter Droplets. Cell 161, 1202-1214 (2015).
19. Quake, S. Solving the Tyranny of Pipetting. arXiv (2018).
20. Ismagilov, R. F., Ng, J. M., Kenis, P. J. & Whitesides, G. M. Microfluidic arrays of fluid-fluid diffusional contacts as detection elements and combinatorial tools. Anal. Chem. 73, 5207-5213 (2001).
21. Zahn, H. et al. Scalable whole-genome single-cell library preparation without preamplification. Nat. Methods 14, 167-173 (2017).
22. Hassibi, A. et al. Multiplexed identification, quantification and genotyping of infectious agents using a semiconductor biochip. Nat. Biotechnol. 36, 738-745 (2018).
23. Dunbar, S. A. Applications of Luminex xMAP technology for rapid, high-throughput multiplexed nucleic acid detection. Clin. Chim. Acta 363, 71-82 (2006).
24. Nguyen, H. Q. et al. Programmable Microfluidic Synthesis of Over One Thousand Uniquely Identifiable Spectral Codes. Adv Opt Mater 5, (2017).
25. Zhao, Y. et al. Microfluidic generation of multifunctional quantum dot barcode particles. J. Am. Chem. Soc. 133, 8790-8793 (2011).
26. Dunbar, S. A. & Li, D. Introduction to Luminex xMAP Technology and Applications for Biological Analysis in China. Asia Pacific Biotech News 14, 26-30 (2010).
27. Untergasser, A. et al. Primer3—new capabilities and interfaces. Nucleic Acids Res. 40, e115-e115 (2012).
28. Bodaghi, S. et al. Could human papillomaviruses be spread through blood? J. Clin. Microbiol. 43, 5428-5434 (2005).
29. Moen, E. M., Huang, L. & Grinde, B. Molecular epidemiology of TTV-like mini virus in Norway. Arch. Virol. 147, 181-185 (2002).
30. Gupta, R. K. et al. HIV-1 drug resistance before initiation or re-initiation of first-line antiretroviral therapy in low-income and middle-income countries: a systematic review and meta-regression analysis. Lancet Infect. Dis. 18, 346-355 (2018).
31. Wensing, A. M. et al. 2017 Update of the Drug Resistance Mutations in HIV-1. Top. Antivir. Med. 24, 132-133 (2017).
32. K. Katoh, D. M. Standley, MAFFT multiple sequence alignment software version 7: improvements in performance and usability. Mol. Biol. Evol. 30, 772-780 (2013).
33. H. Li, Aligning sequence reads, clone sequences and assembly contigs with BWA-MEM (2013), (available at http://arxiv.org/abs/1303.3997).
34. J. Quick et al., Multiplex PCR method for MinION and Illumina sequencing of Zika and other virus genomes directly from clinical samples. Nat. Protoc. 12, 1261-1276 (2017).
35. S.-Y. Rhee et al., Human immunodeficiency virus reverse transcriptase and protease sequence database. Nucleic Acids Res. 31, 298-303 (2003).
36. J. Kehe et al., Massively parallel screening of synthetic microbial communities. PNAS. In Press.
37. M. A. Quail et al., SASI-Seq: sample assurance Spike-Ins, and highly differentiating 384 barcoding for Illumina sequencing. BMC Genomics. 15 (2014), doi:10.1186/1471-2164-15-110.

### Example 3: Region Specific Detection Panel

In this project, a diagnostic panel will be developed for viral species and strains circulating in Honduras. In parallel, Applicants will deploy existing Cas13-based assays for Zika virus detection and dengue serotyping to test patient samples in collaboration with the Universidad Nacional Autónoma de Honduras (UNAH). Hardware will be deployed for multiplexed Cas13-based diagnostics at the UNAH and to train collaborators to use the technology. Successful completion of these aims will produce and validate a multiplexed CRISPR-based detection technology for disease surveillance in a country with many endemic viruses. The work will be a critical first step toward a world in which every infected person who comes into a hospital receives a molecular diagnosis, improving patient care and contributing to public health efforts by providing rich data sets about viral prevalence.

The first goal will be to develop a Cas-13 based viral diagnostic panel for use in Honduras. Utilization of prior Cas13-based viral diagnostics (Myhrvold*, Freije*, et al. Science 2018) and the highly multiplexed microwell array for miniaturizing biochemical assays in nanoliter droplets (Kulesa*, Kehe* et al. PNAS 2018) will provide multiplexed amplification with multiplexed detection using droplets in microwell arrays.

Applicants will design, implement, and validate a diagnostic panel consisting of multiplexed amplification primers and crRNAs targeting a set of 20-30 viral pathogens that are known to circulate in Honduras. This panel will also contain a handful of high-risk viral pathogens that have not been found in Honduras to date, but that would have large public health implications, were they to be detected. While such large-scale assay development would have been cost- and time-prohibitive just last year, the microwell array technology enables development and performance of Cas13 detection assays at scale. It is believed the panel will be the first comprehensive, country-specific viral diagnostic panel. The goals will be development of a multiplexed panel covering at least 20 viruses of interest, with a limit of detection of 100 copies per microliter for each assay and no detectable cross-reactivity, achieving a sensitivity that would be comparable to methods as described in Myhrvold*, Freije*, et al. Science 2018, which allowed detection of virus in patient samples at concentrations as low as 1 copy per microliter. In the second aim, Applicants will deploy Cas13-based detection technology to Honduras, including the comprehensive, multiplexed viral panel. Initial experiments will focus on deploying standard SHERLOCK assays in Honduras, to ensure that the underlying Cas13 technology detects circulating Zika and dengue viruses with high sensitivity (Months 1-8). For the multiplexed panel, the plan is to initially test assays at Broad (Months 1-8), and then bring them to Honduras (Months 9-12) to catch the beginning of the epidemiological season (which typically starts in February). Assembly of the hardware setup will be performed at Broad in months 5-8 to ensure that Applicants have a system with similar sensitivity and specificity to the existing microscope hardware.

The second aim will benefit from existing efforts to deploy Cas13-based viral diagnostics for Zika and dengue in Honduras; a pilot study is underway. Accomplishing the aim would enable an extensive demonstration of traditional and multiplexed CRISPR-based diagnostics in Honduras, spearheading the use of CRISPR-based diagnostics for viral surveillance across the world.

While potential design challenges include variable sensitivity from virus to virus and cross-reactivity between viral species, the methods disclosed herein utilizing the microwell array allows one cycle of assay testing to take only a day or two, so assays can be rapidly optimized during this project. It is expected to detect understudied viruses using the diagnostic panel, with analysis of dozens of samples (50-100). However, the extent to which understudied viruses may be observed represents an open research question. Advantageously, the approaches disclosed herein will develop and use droplets in the microwell arrays, a 4-color fluorescent microscope with an automated stage will be assembled and tested at Broad and deployed to Honduras. The methods allow use of a no-frills microscope that achieves the fluorescence sensitivity and spatial resolution necessary to image droplets in microwell arrays, thereby maximizing hardware robustness while decreasing costs.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method for detecting target molecules comprising:
combining the first set and second set of droplets into a pool of droplets, the first set of droplets comprising a detection CRISPR system comprising a Cas protein and one or more guide molecules designed to bind to corresponding target molecules, a masking construct and an optical barcode, and the second set of droplets comprising a sample and optionally an optical barcode;
flowing the pool of droplets onto a microfluidic device comprising an array of microwells and at least one flow channel beneath the microwells, the microwells sized to capture at least two droplets;
detecting the optical barcodes of the droplets captured in each microwell;
merging the droplets captured in each microwell to form merged droplets in each microwell, at least a subset of the merged droplets comprising a detection CRISPR system and a target sequence;
initiating a detection reaction; and
measuring a detectable signal of each merged droplet at one or more time periods, optionally continuously.

2. The method of claim 1, further comprising a step of amplifying the target molecules, optionally wherein the amplifying comprises nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), or ramification amplification method (RAM), preferably wherein the amplifying is performed with RPA or PCR.

3. (canceled)

4. (canceled)

5. The method of claim 1, wherein the target molecules are contained in a biological sample or an environmental sample, optionally wherein the biological sample is blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, or fluid obtained from a joint, or a swab of skin or mucosal membrane surface, optionally wherein the sample is from a human.

6. (canceled)

7. (canceled)

8. The method of claim 1, wherein the one or more guide are RNAs designed to bind to corresponding target molecules comprise a (synthetic) mismatch, optionally wherein said mismatch is up- or downstream of a SNP or other single nucleotide variation in said target molecule.

9. (canceled)

10. The method of claim 1, wherein the one or more guide RNAs are designed to detect a single nucleotide polymorphism in a target RNA or DNA, or a splice variant of an RNA transcript, optionally wherein the one or more guide RNAs are designed to detect drug resistance SNPs in a viral infection.

11. (canceled)

12. The method of claim 1, wherein the one or more guide RNAs are designed to bind to one or more target molecules that are diagnostic for a disease state, optionally wherein the disease state is characterized by the presence or absence of drug resistance or susceptibility gene or transcript or polypeptide.

13. (canceled)

14. The method of claim 1, wherein the one or more guide RNAs are designed to distinguish between one or more microbial strains.

15. The method of claim 12, wherein the disease state is an infection, optionally wherein the infection is caused by a virus, a bacterium a fungus, a protozoa, or a parasite.

16. (canceled)

17. The method of claim 15, wherein the one or more guide RNAs comprise at least 90 guide RNAs.

18. The method of claim 1, wherein the CRISPR protein is an RNA-targeting protein, a DNA-targeting protein, or a combination thereof.

19. The method of claim 18, wherein the RNA targeting protein comprises one or more HEPN domains, optionally wherein the one or more HEPN domains comprise a RxxxxH motif sequence, optionally wherein the RxxxH motif comprises a R{N/H/K]X₁X₂X₃H sequence, optionally wherein X₁ is R, S, D, E, Q, N, G, or Y, and X₂ is independently I, S, T, V, or L, and X₃ is independently L, F, N, Y, V, I, S, D, E, or A.

20. (canceled)

21. (canceled)

22. (canceled)

23. The method of claim 19, wherein the CRISPR RNA-targeting protein is C2c2.

24. The method of claim 18, wherein the CRISPR protein is a DNA-targeting protein, optionally wherein the CRISPR protein comprises a RuvC-like domain, optionally wherein the DNA-targeting protein is a Type V protein, optionally wherein the DNA-targeting protein is a Cas12, optionally wherein the Cas12 is Cpf1, C2c3, C2c1 or a combination thereof.

25. (canceled)

26. (canceled)

27. (canceled)

28. (canceled)

29. The method of claim 1, wherein the masking construct is RNA-based and suppresses generation of a detectable positive signal.

30. The method of claim 29, wherein the RNA-based masking construct suppresses generation of a detectable positive signal by masking the detectable positive signal, or generating a detectable negative signal instead.

31. The method of claim 29, wherein the RNA-based masking construct comprises a silencing RNA that suppresses generation of a gene product encoded by a reporting construct, wherein the gene product generates the detectable positive signal when expressed.

32. The method of claim 29, wherein the RNA-based masking construct is a ribozyme that generates the negative detectable signal, and wherein the positive detectable signal is generated when the ribozyme is deactivated.

33. The method of claim 32, wherein the ribozyme converts a substrate to a first color and wherein the substrate converts to a second color when the ribozyme is deactivated.

34. The method of claim 29, wherein the RNA-based masking agent is an RNA aptamer and/or comprises an RNA-tethered inhibitor, optionally wherein the aptamer or RNA-tethered inhibitor sequesters an enzyme, wherein the enzyme generates a detectable signal upon release from the aptamer or RNA tethered inhibitor by acting upon a substrate,
optionally wherein the aptamer is an inhibitory aptamer that inhibits an enzyme and prevents the enzyme from catalyzing generation of a detectable signal from a substrate or wherein the RNA-tethered inhibitor inhibits an enzyme and prevents the enzyme from catalyzing generation of a detectable signal from a substrate, preferably wherein the aptamer is an inhibitory aptamer that inhibits an enzyme and prevents the enzyme from catalyzing generation of a detectable signal from a substrate or wherein the RNA-tethered inhibitor inhibits an enzyme and prevents the enzyme from catalyzing generation of a detectable signal from a substrate, optionally wherein the enzyme is thrombin, protein C, neutrophil elastase, subtilisin, horseradish peroxidase, beta-galactosidase, or calf alkaline phosphatase, optionally wherein the enzyme is thrombin and the substrate is para-nitroanilide covalently linked to a peptide substrate for thrombin, or 7-amino-4-methylcoumarin covalently linked to a peptide substrate for thrombin,
optionally wherein the aptamer sequesters a pair of agents that when released from the aptamers combine to generate a detectable signal.

35. (canceled)

36. (canceled)

37. (canceled)

38. (canceled)

39. (canceled)

40. The method of claim 29, wherein the RNA-based masking construct comprises an RNA oligonucleotide to which a detectable ligand and a masking component are attached.

41. The method of claim 29, wherein the RNA-based masking construct comprises a nanoparticle held in aggregate by bridge molecules, wherein at least a portion of the bridge molecules comprises RNA, and wherein the solution undergoes a color shift when the nanoparticle is disbursed in solution, optionally wherein the nanoparticle is a colloidal metal, optionally wherein the colloidal metal is colloidal gold.

42. (canceled)

43. (canceled)

44. The method of claim 22, wherein the RNA-based masking construct comprising a quantum dot linked to one or more quencher molecules by a linking molecule, wherein at least a portion of the linking molecule comprises RNA.

45. The method of claim 22, wherein the RNA-based masking construct comprises RNA in complex with an intercalating agent, wherein the intercalating agent changes absorbance upon cleavage of the RNA, optionally wherein the intercalating agent is pyronine-Y or methylene blue.

46. (canceled)

47. The method of claim 22, wherein the detectable ligand is a fluorophore and the masking component is a quencher molecule.

48. The method of claim 1, wherein the detecting the optical barcodes comprises making optical assessments of the droplets in each microwell, optionally wherein the making optical assessments comprises capturing an image of each microwell, optionally wherein the optical barcode is detected using light microscopy, fluorescence microscopy, Raman spectroscopy, or a combination thereof.

49. (canceled)

50. The method of claim 1, wherein the optical barcode comprises a particle of a particular size, shape, refractive index, color, or combination thereof, optionally wherein the particle comprises colloidal metal particles, nanoshells, nanotubes, nanorods, quantum dots, hydrogel particles, liposomes, dendrimers, or metal-liposome particles.

51. (canceled)

52. (canceled)

53. The method of claim 1, wherein each optical barcode comprises one or more fluorescent dyes.

54. The method of claim 53, wherein each optical barcode comprises a distinct ratio of fluorescent dyes.

55. The method of claim 1, wherein the detectable signal is a level of fluorescence.

56. The method of claim 1, further comprising the step of applying a set cover solving process.

57. The method of claim 1, wherein the microfluidic device comprises an array of at least 40,000 microwells or at least 190,000 microwells.

58. (canceled)

59. A multiplex detection system comprising:
a detection CRISPR system comprising a Cas protein and one or more guide RNAs designed to bind to corresponding target molecules, an RNA-based masking construct and an optical barcode;
optional optical barcodes for one or more target molecules;
and a microfluidic device comprising an array of microwells and at least one flow channel beneath the microwells, the microwells sized to capture at least two droplets.

60. A kit comprising the multiplex detection system of claim 59.

61. The method of claim 1, wherein the second set of droplets comprises an optical barcode.

62. The multiplex detection system of claim 59, wherein the system comprises optical barcodes for one or more target molecules.
